(19) United States
(12) Patent Application Publication
(10) Pub. No.: US 2026/0035360 A1
(43) Pub. Date: Feb. 5, 2026

(54) INHIBITORS OF MYC AND USES THEREOF

(71) Applicant: Northwestern University, Evanston, IL (US)

(72) Inventors: Gary E. Schiltz, Evanston, IL (US); Sarki Abba Abdulkadir, Evanston, IL (US)

(21) Appl. No.: 19/099,534

(22) PCT Filed: Jul. 31, 2023
(86) PCT No.: PCT/US2023/071380

§ 371 Date: Jan. 29, 2025

Related U.S. Application Data
(60) Provisional application No. 63/369,892, filed on Jul. 29, 2022.

(51) Int. Cl. C07D 403/14; A61K 31/40; A61K 31/4155; A61K 31/4192; A61K 31/4196; A61K 31/427; A61K 31/4439; A61K 31/454; A61K 31/4725; A61K 31/496; A61K 31/519; A61K 31/675; A61P 35/00; C07D 207/34; C07D 231/12; C07D 231/16; C07D 231/38; C07D 249/06; C07D 249/08; C07D 401/04; C07D 401/14; C07D 403/12; C07D 405/04; C07D 417/12; C07D 417/14; C07D 487/04; C07F 9/6503

(52) U.S. Cl. CPC C07D 403/14; A61K 31/40; A61K 31/4155; A61K 31/4192; A61K 31/4196; A61K 31/427; A61K 31/4439; A61K 31/454; A61K 31/4725; A61K 31/496; A61K 31/519; A61K 31/675; A61P 35/00; C07D 207/34; C07D 231/12; C07D 231/16; C07D 231/38; C07D 249/06; C07D 249/08; C07D 401/04; C07D 401/14; C07D 403/12; C07D 405/04; C07D 417/12; C07D 417/14; C07D 487/04; C07F 9/65031

(57) ABSTRACT

Disclosed are substituted heterocyclic compounds and proteolysis-targeting chimeric molecules (PROTACs). The substituted heterocycles disclosed herein are shown to be useful in inhibiting c-MYC. The disclosed PROTACs are shown to induce degradation of c-MYC protein. The substituted heterocyclic compounds and proteolysis-targeting chimeric molecules (PROTACs) disclosed, herein may be utilized as therapeutics for treating cancer and cell proliferative disorders.

### CROSS-REFERENCE TO RELATED PATENT APPLICATIONS

This application claims the benefit of priority of U.S. Provisional Patent Application Ser. No. 63/369,892, filed Jul. 29, 2022, the contents of which is incorporated by reference in its entirety.

### STATEMENT REGARDING FEDERALLY SPONSORED RESEARCH OR DEVELOPMENT

This invention was made with government support under grant numbers CA180995 and CA257258 awarded by the National Institutes of Health. The government has certain rights in the invention.

### BACKGROUND

The field of the invention relates to substituted heterocycles as c-MYC targeting agents and proteolysis-targeting chimeric molecules (PROTACs) that induce degradation of c-MYC protein. In particular, the field of the invention relates to substituted pyrazoles, imidazoles, or triazoles as c-MYC targeting agents, and PROTACs that target c-MYC for degradation, which may be used for the treatment of cell proliferation diseases and disorders such as cancer.

The c-MYC oncogene is deregulated and plays a causal role in a majority of human cancer and c-MYC inhibition profoundly affects tumor growth or survival in multiple models. MYC is the most common oncogene involved in human cancers and is overexpressed in up to half of all cancers. Therefore, developing c-MYC inhibitors is among the most attractive potential anti-cancer strategies. Unfortunately, due to the difficulty in targeting transcription factors with small molecules, c-MYC is currently regarded as “undruggable.” Compounds selectively targeting c-MYC-driven cell proliferation and interfering with binding of c-MYC to DNA can be found in, e.g., U.S. Publication No. 2017/0253581, published on Sep. 7, 2017, U.S. Publication No. 2019/0062281, published on Feb. 28, 2019, U.S. Publication No. 2020/0390894, published on Dec. 17, 2020, and U.S. Publication No. 2020/0392116, published on Dec. 17, 2020, the content of which is incorporated herein by reference in its entirety.

Proteolysis-targeting chimeric molecules (PROTACs) are an emerging technology that may be utilized to target previously “undruggable” targets, such as transcription factors and non-enzymatic proteins. (See, e.g., An et al., “Small-molecule PROTACs: An emerging and promising approach for the development of targeted therapy drugs,” EBioMedicine. 2018 October; 36:553-562; and Gu et al., “PROTACs: An Emerging Targeting Technique for Protein Degradation in Drug Discovery,” Bioessays. 2018 April; 40(4): e1700247, the contents of which are incorporated herein by reference in their entireties). PROTACs are chimeric molecules that may be characterized as “hetero-bifunctional” in that PROTACs include a ligand for recruiting an E3 ubiquitin ligase, a linker, and another ligand to bind with the protein targeted for degradation. Designed as such, PROTACs “hijack” the E3 ubiquitin ligase to the protein which is targeted for protein degradation via ubiquitination, even if the targeted protein is not a physiological substrate for degradation via the ubiquitin-proteasome system.

Here, we disclose an approach to selectively targeting c-MYC-driven cell proliferation and interfering with binding of c-MYC to DNA. PROTACs that induce degradation of c-MYC protein are also disclosed.

### SUMMARY

Disclosed are substituted heterocycles which may be utilized as c-MYC targeting agents. The substituted heterocycles may include substituted pyrazoles, substituted imidazoles, and substituted triazoles. The disclosed heterocycles may be used in pharmaceutical compositions and methods for treating cell proliferative disorders such as cancer.

The disclosed substituted heterocycles may include substituted pyrazoles, imidazoles, and triazoles having a formula I:

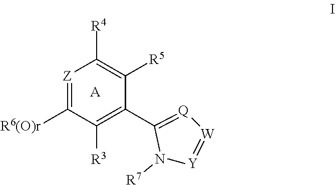

wherein
W is CR⁸ or N;
Y is CH or N;
Q is CR² or N;
Z is C(Alk²)_{q}(X)ₚ(Alk¹)ₙR¹ or N;
R¹ is hydrogen, halo, alkyl, aryl, benzyl, heteroaryl, cycloalkyl, alkoxy, or cycloheteroalkyl, wherein R¹ is optionally substituted at one or more positions with one or more of alkyl, alkoxy, cycloalkyl, haloalkyl, haloalkoxy, aryl, hydroxyl, halo, cyano, amido, carboxyl, —(CH₂)—NH—R⁹, —(O(CH₂)₂)ₘ—R¹⁵, or —OR¹¹;
Alk¹ is straight-chain or branched alkylenyl, or a cycloalkylenyl;
n is 0, 1, or 2;
p is 0 or 1;
Alk² is straight-chain or branched alkylenyl;
q is 0 or 1;
r is 0 or 1;
m is an integer selected from 1 to 20;
X is O or NR¹³;
R² is hydrogen or halo;
R³ is alkyl, alkoxy, haloalkyl, haloalkoxy, hydroxyl, —O—C(O)-alkyl, or halo;
R⁴ is hydrogen, halo, amino, alkyl, or haloalkyl; or R⁴ is aryl or benzyl optionally substituted at one or more ring positions with one or more of alkyl, alkoxy, haloalkyl, haloalkoxy, aryl, hydroxyl, halo, cyano, amido, carboxyl, aryloxy, and alkylaryloxy; or R⁴ is alkyl optionally substituted with halo-substituted aryloxy; or R⁴ together with R¹ form a cycloheteroalkyl fused to ring A, wherein the cycloheteroalkyl fused to ring A is optionally substituted with aryl or alkylaryl optionally substituted with halogen;
R⁵ is hydrogen, halo, alkyl, aryl, alkylaryl, heteroaryl, cycloalkyl, or cycloheteroalkyl, optionally R⁵ is substituted at one or more positions with one or more of alkyl, alkoxy, haloalkyl, haloalkoxy, hydroxyl, halo, cyano, carboxyamido, carboxy, aryloxy, and heteroaryloxy;
R⁶ is hydrogen, halo, alkyl, aryl, alkylaryl, heteroaryl, cycloalkyl, or cycloheteroalkyl, optionally R⁶ is substituted at one or more positions with one or more of alkyl, alkoxy, haloalkyl, haloalkoxy, hydroxyl, halo, cyano, carboxyamido, carboxy, aryloxy, and heteroaryloxy;
R⁷ is alkyl;
R⁸ is hydrogen, cyano, amino, alkyl, alkoxy, haloalkyl, haloalkoxy, aryl, benzyl, hydroxyl, halo, amido, and carboxyl;
R¹ is alkyl optionally substituted with one or more of —P(O)(OR¹)₂;
R¹⁰ is hydrogen or alkyl;
R¹¹ is alkyl optionally substituted with one or more of —P(O)(OR¹²)₂;
R¹² is hydrogen or alkyl;
R¹³ is hydrogen, alkyl, or —C(O)R¹⁴;
R¹⁴ is aryl optionally substituted at one or more ring positions with one or more of alkyl, alkoxy, haloalkyl, haloalkoxy, aryl, hydroxyl, halo, cyano, amido, and carboxyl;
R¹⁵ is OR¹⁶, —OS(O)₂—R¹⁶, or NR¹⁷R¹⁸;
R¹⁶ is alkyl or aryl, wherein R¹⁶ is optionally substituted at one or more positions with one or more of alkyl, alkoxy, haloalkyl, haloalkoxy, aryl, hydroxyl, halo, cyano, amido, and carboxyl; and
R¹⁷ and R¹⁸ are independently hydrogen or alkyl.

The disclosed compounds may exhibit one or more biological activities. The disclosed compounds may inhibit binding of the Myc/Max complex to DNA (e.g., in a DNA gel shifting assay). The disclosed compounds may not produce significant DNA damage (e.g., in an rH2AX staining assay at a concentration greater than about 0.001 μM, 0.005 μM, 0.01 μM, 0.1 μM, 1.0 μM, 10 μM, 100 μM, or higher). The disclosed compounds may inhibit the growth of cells that express c-MYC (preferably by at a concentration of less than about 100 μM, 50 μM, 10 μM, 1 μM, 0.1 μM, 0.05 μM, 0.01 μM, 0.005 μM, 0.001 μM, or less). The disclosed compounds may not inhibit the growth of cells that do not express c-MYC (preferably at a concentration of greater than about 0.001 μM, 0.005 μM, 0.01 μM, 0.5 μM, 0.1 μM, 1.0 μM, 10 μM, and 100 μM or higher).

Also disclosed are proteolysis-targeting chimeric molecules (PROTACs) that induce degradation of c-MYC protein. The disclosed PROTACs comprise a moiety that binds to c-MYC covalently attached to a moiety that binds to a ubiquitin ligase. The disclosed PROTACs typically include a first targeting moiety that binds to c-MYC (M_{c-MYC}) which may be derived from a substituted heterocycle that binds to c-MYC such as a substituted pyrazole. The first targeting moiety may be covalently attached via a bond or a linker (L) to a second targeting moiety that binds to a ubiquitin ligase such as an E3 ubiquitin ligase (M_{E3}). As such, the disclosed PROTACS may be described as having formula II or a formula M_{c-MYC}-L-M_{E3} or M_{E3}-L-M_{c-MYC}.

The disclosed PROTACs target the E3 ubiquitin ligase moiety to c-MYC which subsequently is ubiquitinated and targeted for degradation. The disclosed PROTACs may be utilized for the treatment of diseases and disorders associated with c-MYC such as cell proliferation diseases and disorders including cancer.

The disclosed PROTACs typically include a first targeting moiety that binds to c-MYC (M_{c-MYC}) which is derived from a substituted heterocycle that binds to c-MYC. Suitable substituted heterocycles that bind to c-MYC may include, but are not limited to substituted pyrazoles.

The c-MYC targeting moiety of the disclosed PROTACs (M_{c-MYC}) typically is linked via a bond or a linker (L) to a second targeting moiety that binds to an E3 ubiquitin ligase (M_{E3}).

Suitable linkers for the disclosed PROTACs may include, but are not limited to linkers comprising a polyethylene glycol moiety.

The E3 ubiquitin ligase targeting moiety of the disclosed PROTACs (M_{E3}) typically binds and/or targets the PROTACs to an E3 ubiquitin ligase. Suitable E3 ubiquitin ligases may include, but are not limited to, Von Hippel-Lindau (VHL) E3 ubiquitin ligase, cereblon (CRBN) E3 ubiquitin ligase, inhibitor of apoptosis protein (IAP) E3 ubiquitin ligase, and mouse double minute 2 homolog (MDM2) E3 ubiquitin ligase. The E3 ubiquitin ligase targeting moiety of the disclosed PROTACs (M_{E3}) typically is derived from a compound that binds to an E3 ubiquitin ligase, for example, as a ligand for an E3 ubiquitin ligase. Suitable ligands may include, but are not limited to, ligands derived from thalidomide, pomalidomide, lenalidomide, VHL ligand 1 (VHL-1), VHL ligand 2 (VHL-2), VH032, VL-269, LCL161, hydroxyproline-based ligands, and HIF-1α-derived (R)-hydroxyproline, including radicalized forms.

The disclosed PROTACs may exhibit one or more biological activities. The disclosed PROTACs may inhibit the growth of cells that express c-MYC (preferably by at a concentration of less than about 100 μM, 50 μM, 10 μM, 1 μM, 0.1 μM, 0.05 μM, 0.01 μM, 0.005 μM, 0.001 μM, or less). The disclosed PROTACs may not inhibit the growth of cells that do not express c-MYC (preferably at a concentration of greater than about 0.001 μM, 0.005 μM, 0.01 μM, 0.5 μM, 0.1 μM, 1.0 μM, 10 μM, and 100 μM or higher).

In some embodiments, the PROTACs have a formula: M_{c-MYC}-L-M_{E3}, wherein M_{c-MYC} is a moiety that binds to c-MYC, L is a bond or a linker covalently attaching M_{MYC} and M_{E3}, and M_{E3} is a moiety that binds to an E3 ubiquitin ligase. In some embodiments, the PROTACs have a formula II:

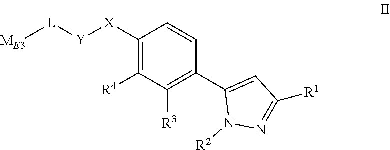

wherein
R¹ is hydrogen, cyano, amino, alkyl, alkoxy, haloalkyl, haloalkoxy, aryl, benzyl, hydroxyl, halo, amido, and carboxyl;
R¹ is alkyl;
R¹ is alkyl, alkoxy, haloalkyl, haloalkoxy, hydroxyl, —O—C(O)-alkyl, or halo;
R⁴ is hydrogen, halo, alkyl, aryl, alkylaryl, heteroaryl, cycloalkyl, or cycloheteroalkyl, optionally R⁴ is substituted at one or more positions with one or more of alkyl, alkoxy, haloalkyl, haloalkoxy, hydroxyl, halo, cyano, carboxyamido, carboxy, aryloxy, and heteroaryloxy;
X is O or NR⁵;
R⁵ is hydrogen, alkyl, or —C(O)R⁶;
R⁶ is aryl optionally substituted at one or more ring positions with one or more of alkyl, alkoxy, haloalkyl, haloalkoxy, aryl, hydroxyl, halo, cyano, amido, and carboxyl;
Y is alkylenyl, arylenyl, benzylenyl, heteroarylenyl, cycloalkylenyl, or cycloheteroalkylenyl;
L is a bond, or a linker selected from the group consisting of —(O(CH₂)₂)ₐ—, —(O(CH₂)₂)ₐ—NH—C(O)-Alk³-, —(O(CH₂)₂)ₐ—C(O)—NH—((CH₂)₂—O)_{b}-Alk³-, —(O(CH₂)₂)ₐ—C(O)—NH-Alk³-C(O)—, —(O(CH₂)₂)ₐ—C(O)—NH—((CH₂)₂—O)_{b}—, —(O(CH₂)₂)ₐ—C(O)—NH—((CH₂)₂—O)_{b}-Alk³-C(O)—, —(O(CH₂)₂)ₐ—C(O)—NH-Alk³-NH—C(O)—CH₂—, and —(O(CH₂)₂)ₐ—C(O)—NH-Alk³;
a is an integer selected from 1-20;
b is an integer selected from 1-20;
Alk³ is straight-chain or branched alkylenyl;
M_{E3} is selected from the group consisting of

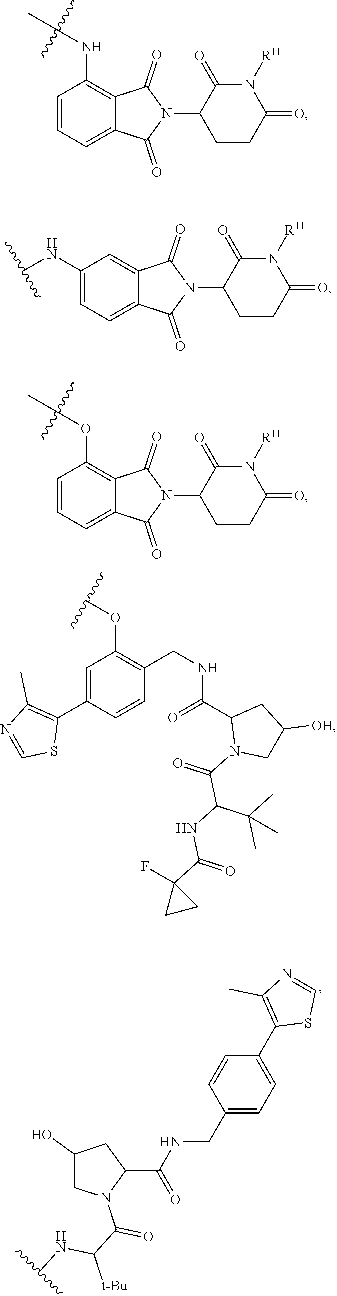

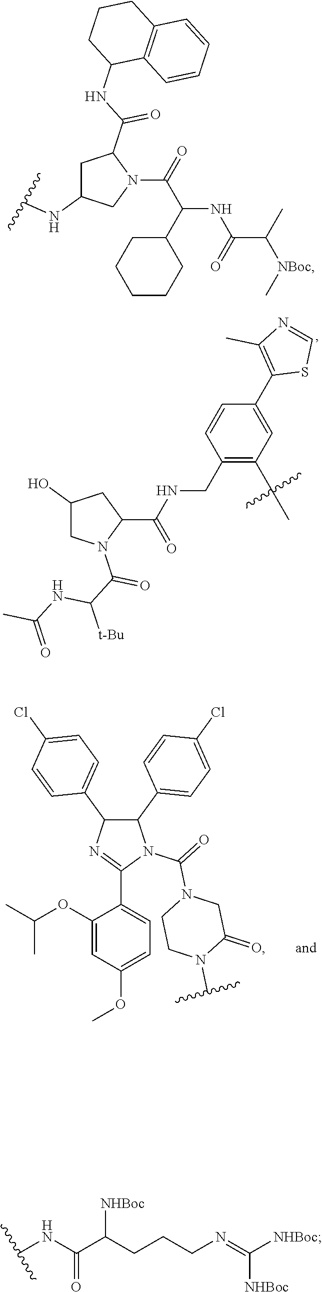

R⁷ is alkyl optionally substituted with one or more of amino or —S(O)₂R⁸;
R⁸ is alkyl or aryl, wherein R⁸ is optionally substituted at one or more positions with one or more of alkyl, alkoxy, haloalkyl, haloalkoxy, aryl, hydroxyl, halo, cyano, amido, and carboxyl, and
R⁹, R¹⁰, and R¹¹ are independently hydrogen or alkyl.

Also disclosed are pharmaceutical compositions comprising the disclosed compounds and/or the disclosed PROTACs, and a suitable pharmaceutical carrier, excipient, or diluent. The disclosed pharmaceutical compositions may comprise an effective amount of the compound and/or the PROTACs for inhibiting the growth of cancer cells when administered to a subject in need thereof.

Also disclosed are methods for treating cell proliferation diseases and disorders such as cancer. The methods may include administering the disclosed compounds, and/or the disclosed PROTACs, or pharmaceutical compositions comprising the disclosed compounds to a subject in need thereof, for example, to a subject having cancer. The disclosed compounds and/or PROTACs or pharmaceutical compositions comprising the disclosed compounds and/or PROTACs may be administered with additional therapeutic agents, optionally in combination, in order to treat cell proliferative diseases and disorders. Cell proliferative diseases and disorders treated by the disclosed methods may include, but are not limited to, cancers selected from the group consisting of multiple myeloma, leukemia, non-small cell lung cancer, colon cancer, cancer of the central nervous system, melanoma, ovarian cancer, renal cancer, prostate cancer, and breast cancer.

### BRIEF DESCRIPTION OF THE DRAWINGS

FIG. 1 depicts the 19F NMR of the compound A4BC1R2 (NUCC-0226279).

FIG. 2 shows PK study of NUCC-0226545 by P.O. (“545” stands for compound NUCC-0226545; “975” stands for compound NUCC-0200975.)

FIG. 3 shows PK study of NUCC-0226605 by P. O. (“605” stands for compound NUCC-0226605; “606” stands for compound NUCC-0226606; “975” stands for compound NUCC-0200975.)

FIG. 4 shows PK study of NUCC-0226606 by P.O. (“7037” stands for compound NUCC-0227037; “416” stands for compound NUCC-0202416; “975” stands for compound NUCC-0200975.)

FIG. 5 shows PK study of NUCC-0227037 by P.O. (“7037” stands for compound NUCC-0227037; “975” stands for compound NUCC-0200975.)

FIG. 6 shows PK study of NUCC-0202416 by P.O. (“416” stands for compound NUCC-0202416; “975” stands for compound NUCC-0200975.)

FIG. 7A depicts the tumor volume change in the tested mice after oral administration of 100 mg/kg MYCi 975 to the mice once a day. (“MYCi 975” stands for compound NUCC-0200975.)

FIG. 7B depicts the tumor volume change in the tested mice after oral administration of 100 mg/kg MYCi 605 to the mice once a day. (“MYCi 605” stands for compound NUCC-0226605.)

FIG. 7C depicts the tumor volume change in the tested mice after oral administration of 30 mg/kg and 10 mg/kg MYCi 605 to the mice once a day. (“MYCi 605” stands for compound NUCC-0226605.)

FIG. 7D depicts the tumor volume change in the tested mice after oral administration of 100 mg/kg, 30 mg/kg, and 10 mg/kg MYCi 606 to the mice once a day. (“MYCi 606” stands for compound NUCC-0226606.)

FIG. 7E depicts the tumor volume change in the tested mice after oral administration of 100 mg/kg, 30 mg/kg, and 10 mg/kg MYCi 7037 to the mice once a day. (“MYCi 7037” stands for compound NUCC-0227037.)

FIG. 8A depicts the body weight change in the tested mice after oral administration of 100 mg/kg MYCi 975 to the mice once a day. (“MYC) 975” stands for compound NUCC-0200975.)

FIG. 8B depicts the body weight change in the tested mice after oral administration of 100 mg/kg MYCi 605 to the mice once a day. (“MYCi 605” stands for compound NUCC-0226605.)

FIG. 8C depicts the body weight change in the tested mice after oral administration of 30 mg/kg and 10 mg/kg MYCi 605 to the mice once a day. (“MYCi 605” stands for compound NUCC-0226605.)

FIG. 8D depicts the body weight change in the tested mice after oral administration of 100 mg/kg, 30 mg/kg, and 10 mg/kg MYCi 606 to the mice once a day. (“MYCi 606” stands for compound NUCC-0226606.)

FIG. 8E depicts the tumor volume change in the tested mice after oral administration of 100 mg/kg, 30 mg/kg, and 10 mg/kg MYCi 7037 to the mice once a day. (“MYCi 7037” stands for compound NUCC-0227037.)

FIG. 9 shows the 1H NMR of NUCC-0227247 (i.e., P2-2203-1).

FIG. 10 shows the ¹H NMR of NUCC-0227245 (i.e., P2-2203-2).

FIG. 11 shows the ¹H NMR of NUCC-0227246 (i.e., P2-2203-2A).

FIG. 12 shows the ¹H NMR of NUCC-0227244 (i.e., P2-2112-3).

### DETAILED DESCRIPTION

The present invention is described herein using several definitions, as set forth below and throughout the application.

Unless otherwise specified or indicated by context, the terms “a”, “an”, and “the” mean “one or more.” For example, “a compound” should be interpreted to mean “one or more compounds.”

As used herein, “about,” “approximately.” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising.” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, a “subject” may be interchangeable with “patient” or “individual” and means an animal, which may be a human or non-human animal, in need of treatment.

A “subject in need of treatment” may include a subject having a disease, disorder, or condition that is responsive to therapy with a substituted heterocycle such as the presently disclosed substituted pyrazoles, substituted imidazoles, and substituted triazoles, or a proteolytic-targeted chimeric molecule (PROTAC), which is targeted to c-MYC for degradation of c-MYC. For example, a “subject in need of treatment” may include a subject having a cell proliferative disease, disorder, or condition such as cancer (e.g., cancers such as multiple myeloma, leukemia, non-small cell lung cancer, colon cancer, cancer of the central nervous system, melanoma, ovarian cancer, renal cancer, prostate cancer, and breast cancer).

As used herein, the phrase “effective amount” shall mean that drug dosage that provides the specific pharmacological response for which the drug is administered in a significant number of subject in need of such treatment. An effective amount of a drug that is administered to a particular subject in a particular instance will not always be effective in treating the conditions/diseases described herein, even though such dosage is deemed to be a therapeutically effective amount by those of skill in the art.

### Chemical Entities

New chemical entities and uses for chemical entities are disclosed herein. The chemical entities may be described using terminology known in the art and further discussed below.

As used herein, a wavy line “ [symbol] ” may be used to designate the point of attachment for any radical group or substituent group.

The term “alkyl” as contemplated herein includes a straight-chain or branched alkyl radical in all of its isomeric forms, such as a straight or branched group of 1-12, 1-10, or 1-6 carbon atoms, referred to herein as C₁-C₁₂-alkyl, C₁-C₁₀-alkyl, and C₁-C₆-alkyl, respectively. The number of carbon atoms in the alkyl group can be specified using the Cx-Cy nomenclature where x and y are integers specifying the number of carbon atoms.

The term “alkylenyl” refers to a diradical of straight-chain or branched alkyl group (i.e., a diradical of straight-chain or branched C₁-C₆ alkyl group). Exemplary alkylenyl groups include, but are not limited to —CH₂—, —CH₂CH₂—, —CH₂CH₂CH₂—, —CH(CH₃)CH₂—, —CH₂CH(CH₃)CH₂—, —CH(CH₂CH₃)CH₂—, and the like.

The term “halo” or “halogen” refers to a radical of —F, —Cl, —Br, or —I.

The term “haloalkyl” refers to an alkyl group that is substituted with at least one halogen. For example, —CH₂F, —CHF₂, —CF₃, —CH₂CF₃, —CF₂CF₃, and the like.

The terms “alkoxy” or “alkoxyl” are art-recognized and refer to an alkyl group, as defined above, having an oxygen radical attached thereto. Representative alkoxy groups include methoxy, ethoxy, tert-butoxy and the like.

The term “haloalkoxy” refers to an alkoxy group, as defined above, that is substituted with at least one halogen.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. The number of carbon atoms in the cycloalkyl group can be specified using the Cx-Cy nomenclature where x and y are integers specifying the number of carbon atoms. Unless specified otherwise, cycloalkyl groups are optionally substituted at one or more ring positions with, for example, alkanoyl, alkoxy, alkyl, haloalkyl, alkenyl, alkynyl, amido or carboxyamido (or amidocarboxyl), amidino, amino, aryl, arylalkyl, azido, carbamate, carbonate, carboxy, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloheteroalkyl” refers to a monovalent saturated cyclic, bicyclic, or bridged cyclic hydrocarbon group of 3-12, 3-8, 4-8, or 4-6 carbons in which at least one carbon of the cycloalkane is replaced with a heteroatom such as, for example, N, O, and/or S.

The term “cycloheteroalkylenyl” as used herein refers to a diradical of the cycloheteroalkyl group as defined above.

The term “cycloalkylenyl” refers to a diradical of a cycloalkyl group, as defined above. Exemplary cycloalkylenyl groups include, but are not limited to

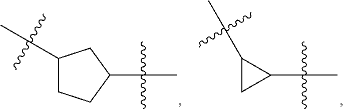

and the like.

The term “aryl” is art-recognized and refers to a carbocyclic aromatic group containing, e.g., 5-12, 6-10, or 5-8 carbons. The number of carbon atoms in the aryl group can be specified using the Cx-Cy nomenclature where x and y are integers specifying the number of carbon atoms. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sμLfhydryl, imino, amido or carboxyamido (or amidocarboxyl), carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The term “arylenyl” as used herein refers to a diradical of the aryl group as defined above.

The term “alkylaryl” refers to a mono-radical having an alkylenyl group attached to an aryl group.

The term “aryloxy” is art-recognized and refers to an aryl group, as defined above, having an oxygen radical attached thereto. Representative alkoxy groups include phenoxy, and the like.

The term “alkylaryloxy” refers to a mono-radical having an alkylenyl group attached to an aryloxy group.

The term “heteroaryl” as used herein refers to a monocyclic heteroaryl and a bicyclic heteroaryl. The monocyclic heteroaryl is a five- or six-membered ring. The five-membered ring contains two double bonds. The five membered ring may contain at least one heteroatom that is O, S, and/or nitrogen; or one, two, three, or four nitrogen atoms and optionally one oxygen or one sulfur atom. The six-membered ring contains three double bonds and one, two, three or four nitrogen, oxygen, and/or sulfur atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl consists of a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryls include, but are not limited to, benzofuranyl, benzothienyl, benzoxazolyl, benzimidazolyl, benzoxadiazolyl, phthalazinyl, 2,6-dihydropyrrolo[3,4-c]pyrazol-5 (4H)-yl, 6,7-dihydro-pyrazolo[1,5-a]pyrazin-5 (4H)-yl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinolinyl, 2,4,6,7-tetrahydro-5H-pyrazolo[4,3-c]pyridin-5-yl, thiazolo[5,4-b]pyridin-2-yl, thiazolo[5,4-d]pyrimidin-2-yl, and 5,6,7,8-tetrahydroquinolin-5-yl. The monocyclic and bicyclic heteroaryls, including exemplary rings, are optionally substituted unless otherwise indicated. The monocyclic and bicyclic heteroaryls are connected to the parent molecular moiety through any substitutable carbon atom or any substitutable nitrogen atom contained within the ring systems. The nitrogen atom in the heteroaryl rings may optionally be oxidized and may optionally be quarternized.

The term “heteroaryloxy” refers to a heteroaryl group, as defined above, having an oxygen radical attached thereto.

The term “heteroarylenyl” as used herein refers to a diradical of the heteroaryl group as defined above.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using “x-membered to y-membered” nomenclature where x and y are integers specifying the number of ring atoms. For example, a 3-membered to 7-membered heterocyclyl group refers to a saturated or partially unsaturated 3- to 7-membered ring structure containing one to four heteroatoms, such as nitrogen, oxygen, and sulfur. The designation indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines (e.g., mono-substituted amines or di-substituted amines), wherein substituents may include, for example, alkyl, cycloalkyl, heterocyclyl, alkenyl, and aryl.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “oxo” refers to a divalent oxygen atom

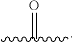 [SMILES: *=O]

The term “carboxamido” as used herein refers to the radical —C(O)NRR′, where R and R′ may be the same or different. R and R′, for example, may be independently hydrogen, alkyl, aryl, arylalkyl, cycloalkyl, formyl, haloalkyl, heteroaryl, or heterocyclyl.

The term “carboxy” or “carboxyl” as used herein refers to the radical-COOH or its corresponding salts, e.g, —COONa, etc.

The term “amide” or “amido” or “amidyl” as used herein refers to a radical of the form R¹C(O)N(R²)—, —R¹C(O)N(R²)R³—, —C(O)NR²R³, or —C(O)NH2, wherein R¹, R² and R³, for example, are each independently hydrogen, alkyl, alkoxy, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “benzyl” as used herein refers to a group of —C₆H₄—CH₂— (i.e.

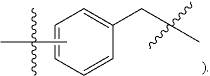

The term “benzylenyl” as used herein refers to a diradical of the benzyl group as defined above.

The term “cyano” refers to a substituent of “—CN”.

The term “hydroxyl” refers to the substituent of “—OH”.

The compounds and molecules (e.g., PROTACs) of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds and molecules may be designated by the symbols “R” or “S,” or “+” or “−” depending on the configuration of substituents around the stereogenic carbon atom and or the optical rotation observed. The present invention encompasses various stereo isomers of these compounds and molecules and mixtures thereof. Stereoisomers include enantiomers and diastereomers. Mixtures of enantiomers or diastereomers may be designated (±)” in nomenclature, but the skilled artisan will recognize that a structure may denote a chiral center implicitly. It is understood that graphical depictions of chemical structures. e.g., generic chemical structures, encompass all stereoisomeric forms of the specified compounds and molecules, unless indicated otherwise. Also contemplated herein are compositions comprising, consisting essentially of, or consisting of an enantiopure compound, which composition may comprise, consist essential of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

The formula of the compounds and molecules disclosed herein should be interpreted as encompassing all possible stereoisomers, enantiomers, or epimers of the compounds and molecules unless the formulae indicate a specific stereoisomer, enantiomer, or epimer. The formulae of the compounds and molecules disclosed herein should be interpreted as encompassing salts, esters, amides, or solvates thereof of the compounds and molecules.

### Substituted Heterocycles as MYC Inhibitors

Disclosed herein are substituted heterocycles. The disclosed heterocycles have been shown to inhibit the biological activity of c-MYC. The disclosed substituted heterocycles may include substituted pyrazoles, substituted imidazoles, and substituted triazoles.

In some embodiments, the disclosed substituted heterocycles may have a formula I:

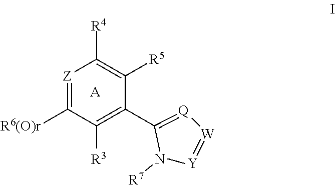

wherein
W is CR⁸ or N;
Y is CH or N;
Q is CR² or N;
Z is C(Alk²)_{q}(X)ₚ(Alk¹)ₙR¹ or N;
R¹ is hydrogen, halo, alkyl, aryl, benzyl, heteroaryl, cycloalkyl, alkoxy, or cycloheteroalkyl, wherein R¹ is optionally substituted at one or more positions with one or more of alkyl, alkoxy, cycloalkyl, haloalkyl, haloalkoxy, aryl, hydroxyl, halo, cyano, amido, carboxyl, —(CH₂)—NH—R⁹, —(O(CH₂)₂)ₘ—R¹⁵, or —OR¹¹;
Alk¹ is straight-chain or branched alkylenyl, or a cycloalkylenyl;
n is 0, 1, or 2;
p is 0 or 1;
Alk² is straight-chain or branched alkylenyl;
q is 0 or 1;
r is 0 or 1;
m is an integer selected from 1 to 20;
X is O or NR 13;
R² is hydrogen or halo;
R³ is alkyl, alkoxy, haloalkyl, haloalkoxy, hydroxyl, —O—C(O)-alkyl, or halo;
R⁴ is hydrogen, halo, amino, alkyl, or haloalkyl; or R⁴ is aryl or benzyl optionally substituted at one or more ring positions with one or more of alkyl, alkoxy, haloalkyl, haloalkoxy, aryl, hydroxyl, halo, cyano, amido, carboxyl, aryloxy, and alkylaryloxy; or R⁴ is alkyl optionally substituted with halo-substituted aryloxy; or R⁴ together with R¹ form a cycloheteroalkyl fused to ring A, wherein the cycloheteroalkyl fused to ring A is optionally substituted with aryl or alkylaryl optionally substituted with halogen;
R⁵ is hydrogen, halo, alkyl, aryl, alkylaryl, heteroaryl, cycloalkyl, or cycloheteroalkyl, optionally R⁵ is substituted at one or more positions with one or more of alkyl, alkoxy, haloalkyl, haloalkoxy, hydroxyl, halo, cyano, carboxyamido, carboxy, aryloxy, and heteroaryloxy;
R⁶ is hydrogen, halo, alkyl, aryl, alkylaryl, heteroaryl, cycloalkyl, or cycloheteroalkyl, optionally R⁶ is substituted at one or more positions with one or more of alkyl, alkoxy, haloalkyl, haloalkoxy, hydroxyl, halo, cyano, carboxyamido, carboxy, aryloxy, and heteroaryloxy;
R⁷ is alkyl;
R⁸ is hydrogen, cyano, amino, alkyl, alkoxy, haloalkyl, haloalkoxy, aryl, benzyl, hydroxyl, halo, amido, and carboxyl;
R⁹ is alkyl optionally substituted with one or more of —P(O)(OR¹⁰)₂;
R¹⁰ is hydrogen or alkyl;
R¹¹ is alkyl optionally substituted with one or more of —P(O)(OR¹²)₂;
R¹² is hydrogen or alkyl;
R¹³ is hydrogen, alkyl, or —C(O)R¹⁴; and
R¹⁴ is aryl optionally substituted at one or more ring positions with one or more of alkyl, alkoxy, haloalkyl, haloalkoxy, aryl, hydroxyl, halo, cyano, amido, and carboxyl;
R¹⁵ is OR¹⁶, —OS(O)₂—R¹⁶, or NR¹⁷R¹⁸;
R¹⁶ is alkyl or aryl, wherein R¹⁶ is optionally substituted at one or more positions with one or more of alkyl, alkoxy, haloalkyl, haloalkoxy, aryl, hydroxyl, halo, cyano, amido, and carboxyl; and
R¹⁷ and R¹⁸ are independently hydrogen or alkyl.

In some embodiments, the disclosed substituted heterocycles may have a structure of formula I, wherein:
R¹ is hydrogen, alkyl, aryl, benzyl, wherein R¹ is optionally substituted at one or more positions with one or more of alkyl, alkoxy, aryl, halo, —(CH₂)—NH—R⁹, or —OR¹¹;
R² is hydrogen or chloro;
R³ is hydroxyl or —OC(O)Me;
R⁴ is hydrogen or halo;
R⁵ is hydrogen or halo;
R⁶ is hydrogen, aryl, benzyl, optionally R⁶ is substituted at one or more positions with one or more of haloalkyl, halo, and cyano;
R⁸ is cyano, amino, haloalkyl, or amido;
R¹⁴ is aryl optionally substituted at one or more ring positions with one or more of haloalkyl or halo; and
the rest of substituents of the compound of formula I are as defined herein.

In some embodiments, the compound is not 4′-chloro-6-((4-chlorobenzyl)oxy)-3-(1-methyl-3-(trifluoromethyl)-1H-pyrazol-5-yl)-3′-(trifluoromethyl)-[1,1′-biphenyl]-2-ol or a compound disclosed in U.S. Publication No. 2017/0253581, published on Sep. 7, 2017, U.S. Publication No. 2019/0062281, published on Feb. 28, 2019, U.S. Publication No. 2020/0390894, published on Dec. 17, 2020, or U.S. Publication No. 2020/0392116, published on Dec. 17, 2020.

In some embodiments, the disclosed substituted heterocycles may have a formula I(a), with the definitions for the substituents same as those in formula I:

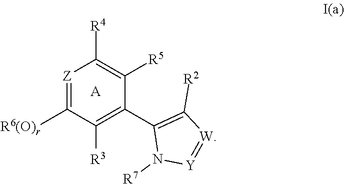

In some embodiments, the disclosed substituted heterocycles may include substituted pyrazoles wherein Y is N and W is CCF₃.

In some embodiments, the disclosed substituted heterocycles may have R³ being hydroxyl.

In some embodiments, the disclosed substituted heterocycles may have r being 0 and R⁶ being phenyl substituted with chloro and trifluoromethyl.

In some embodiments, the disclosed substituted heterocycles may have R² being hydrogen.

In some embodiments, the disclosed substituted heterocycles may be selected from the group consisting of

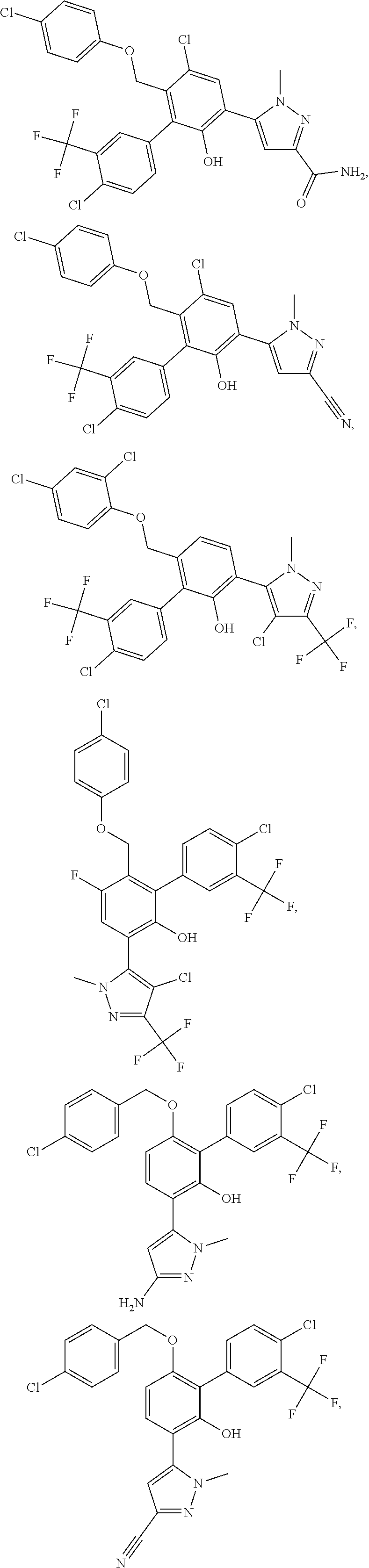

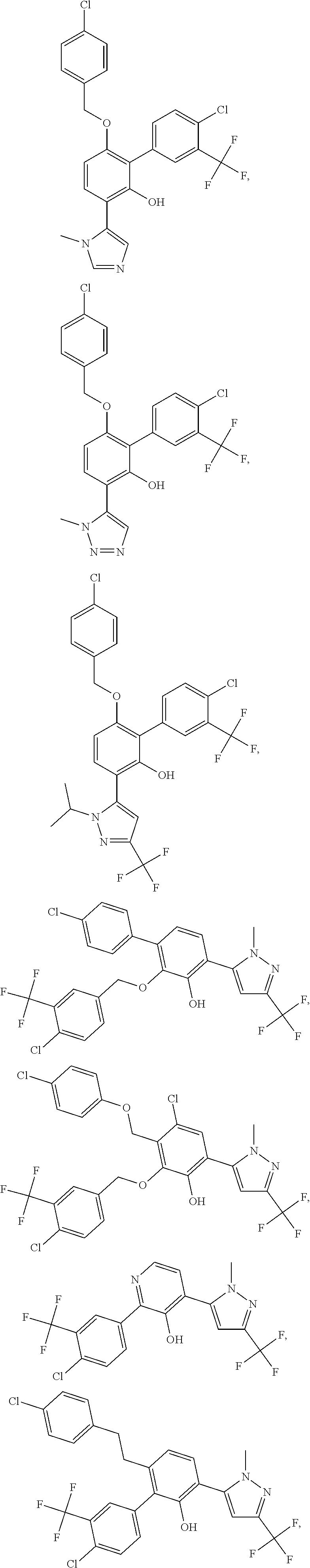

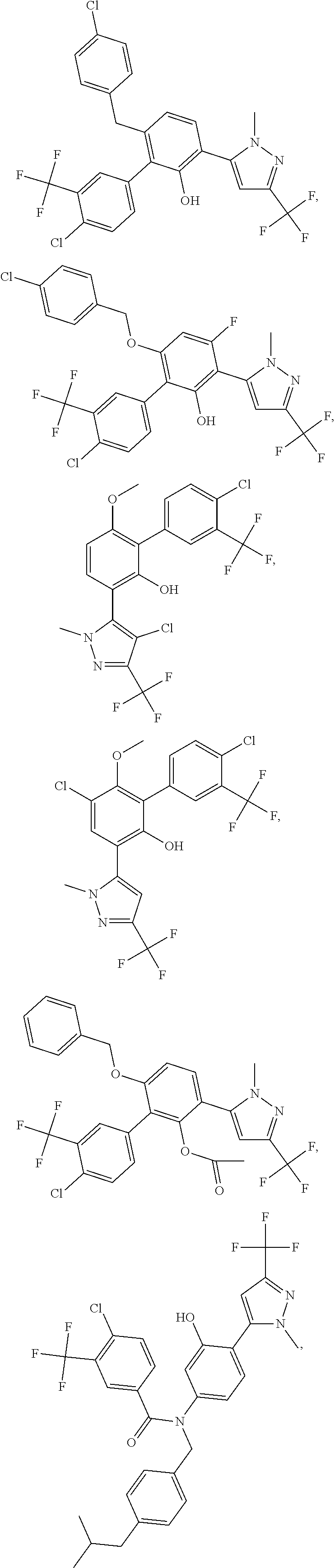

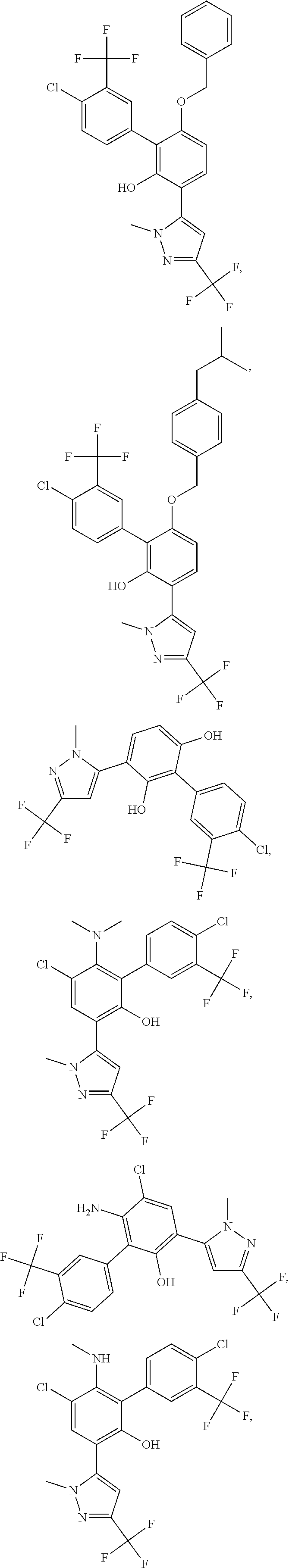

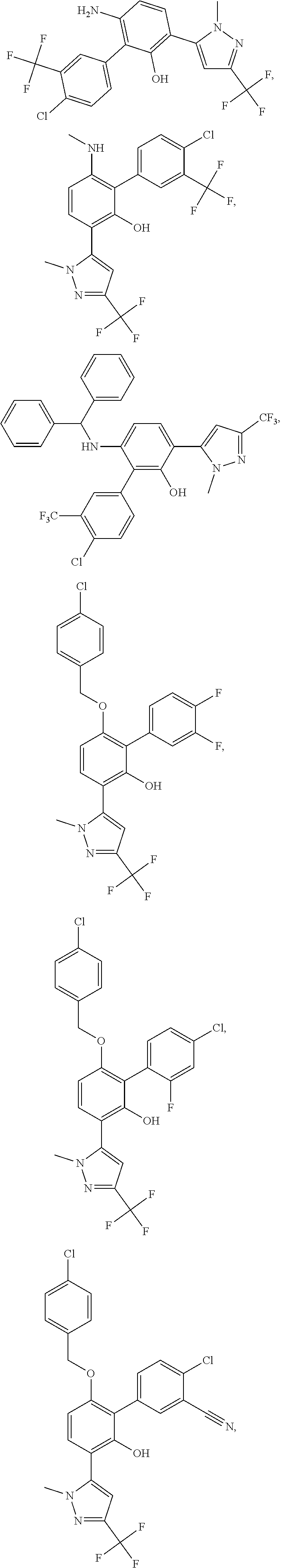

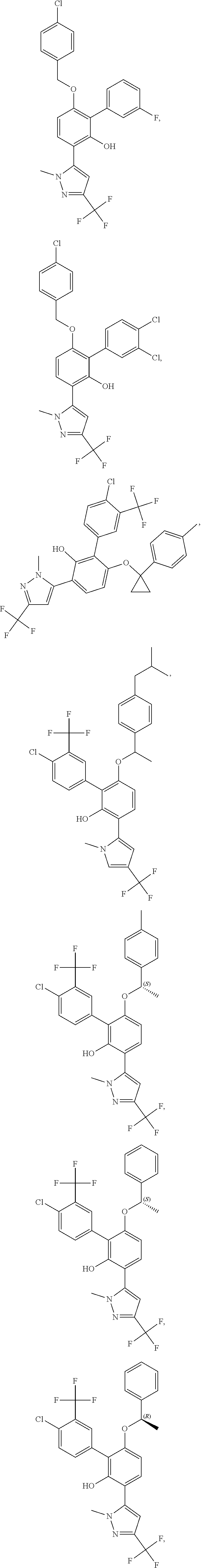

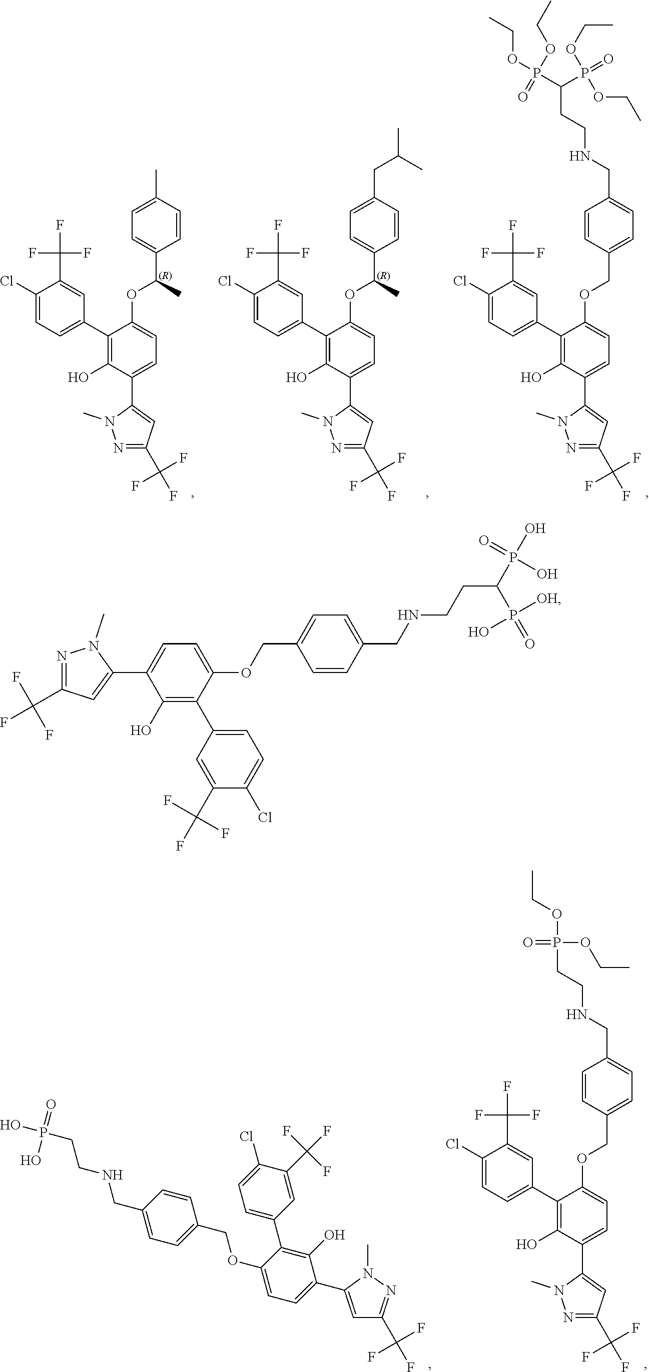

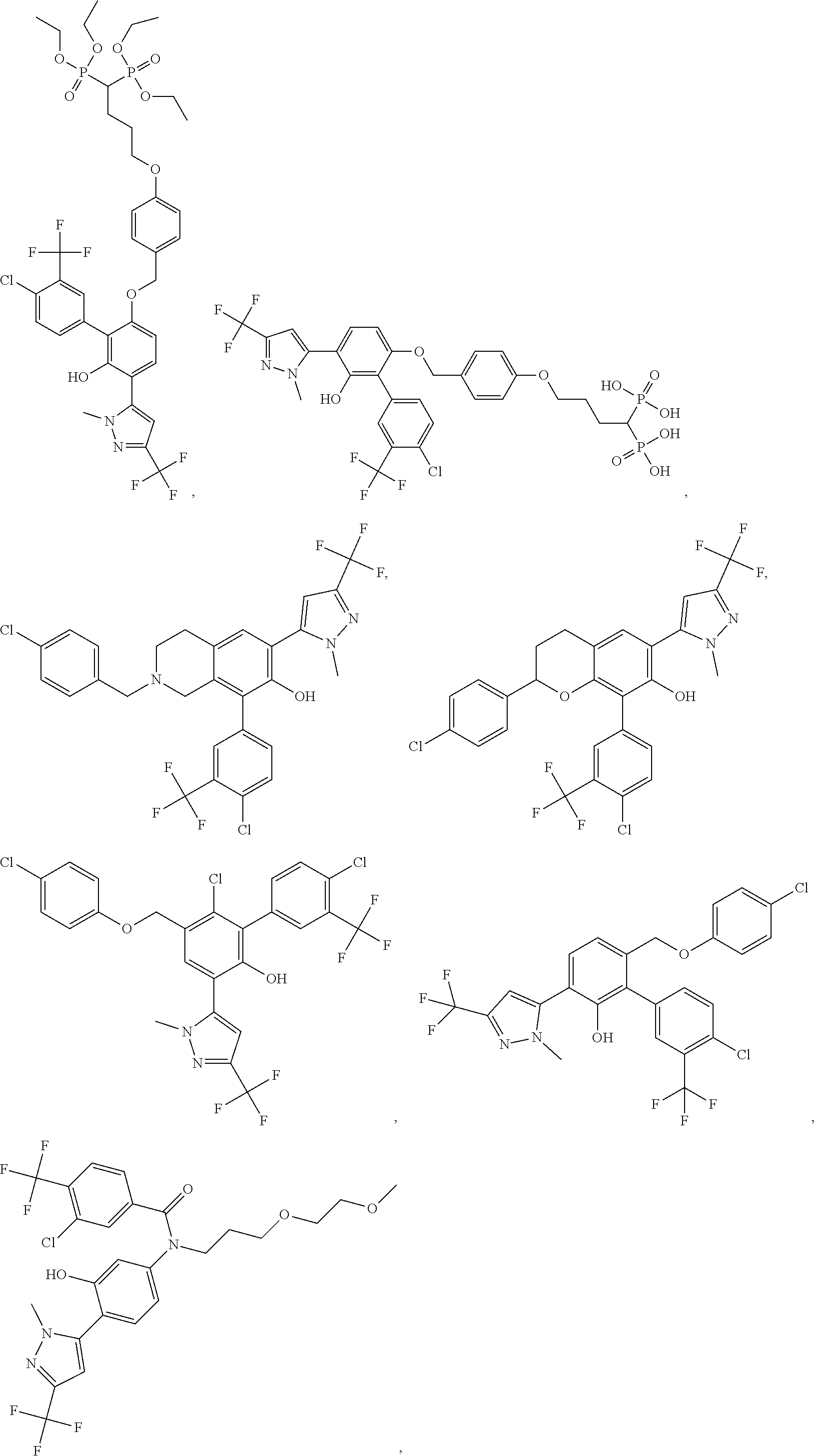

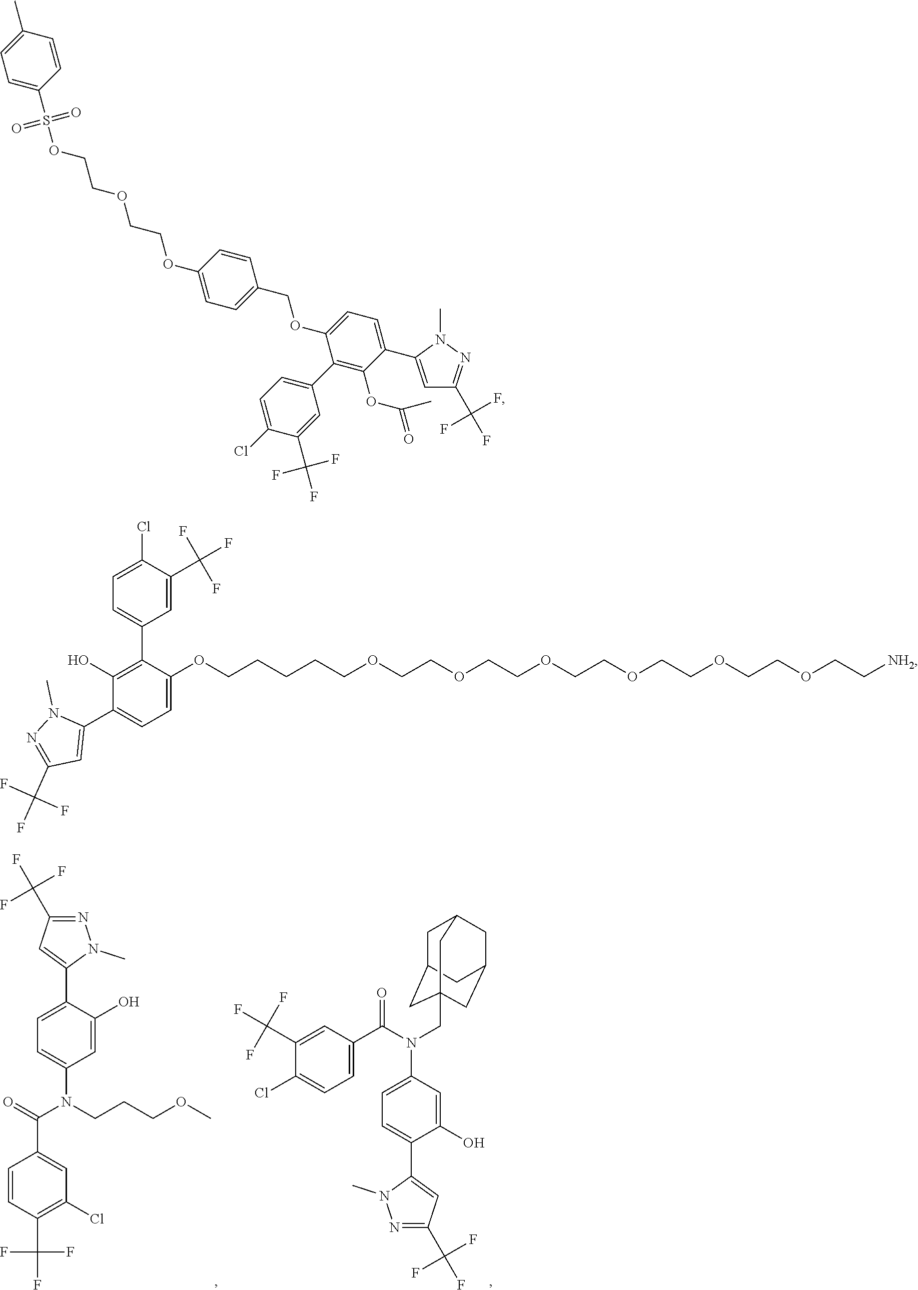

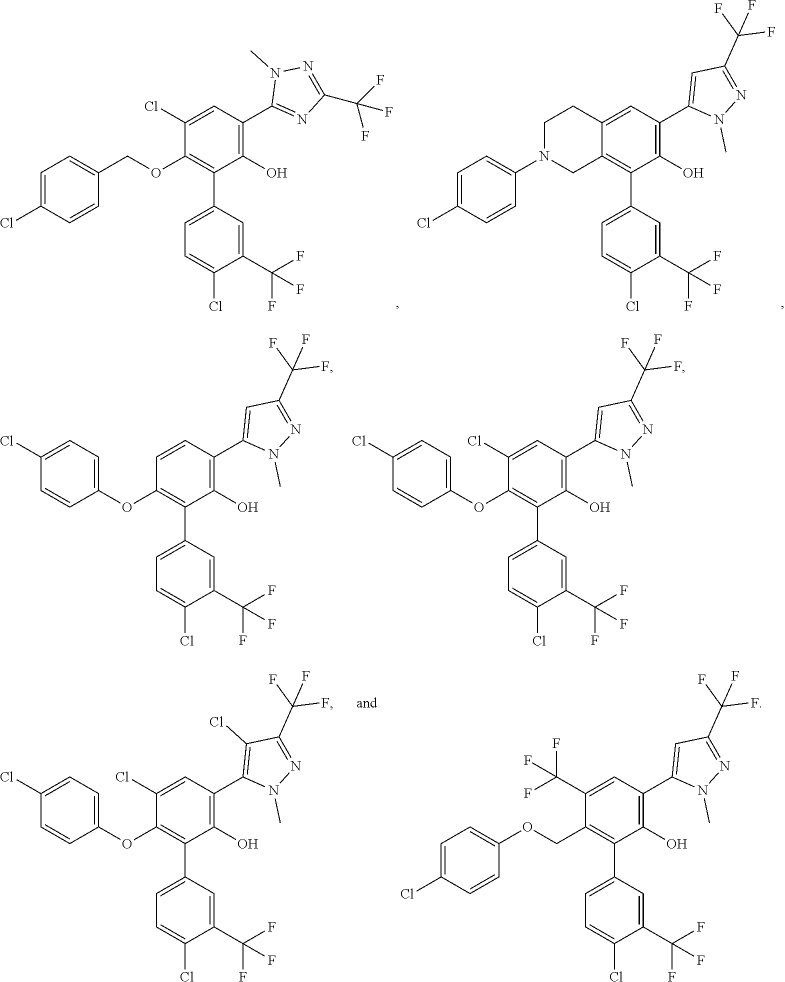

Proteolytic-Targeting Chimeric Molecules (PROTACs) that Induce Degradation of c-MYC Protein

Also disclosed herein are proteolytic-targeted chimeric molecules (PROTACs) that induce degradation of c-MYC protein. In some embodiments, the disclosed molecules may be described as having a having a formula: M_{c-MYC}-L-M_{E3} or alternatively M_{E3}-L-M_{c-MYC}, wherein M_{c-MYC} is a moiety that binds to c-MYC, L is a bond or a linker covalently attaching M_{MYC} and M_{E3}, and M_{E3} is a moiety that binds to an E3 ubiquitin ligase. Compounds that bind to c-MYC are disclosed in the prior art and may include, but are not limited to compounds disclosed in U.S. Publication No. 2019/0062281, published on Feb. 28, 2019, the content of which is incorporated herein by reference in its entirety.

In some embodiments of the disclosed PROTACs, the PROTACs have a formula: M_{c-MYC}-L-M_{E3}, wherein M_{c-MYC} is a moiety that binds to c-MYC, L is a bond or a linker covalently attaching M_{MYC} and M_{E3}, and M_{E3} is a moiety that binds to an E3 ubiquitin ligase.

In some embodiments of the disclosed PROTACs, the PROTACs have a formula II:

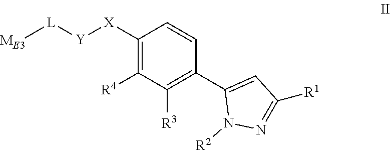

wherein
R¹ is hydrogen, cyano, amino, alkyl, alkoxy, haloalkyl, haloalkoxy, aryl, benzyl, hydroxyl, halo, amido, and carboxyl;
R² is alkyl;
R³ is alkyl, alkoxy, haloalkyl, haloalkoxy, hydroxyl, —O—C(O)-alkyl, or halo;
R⁴ is hydrogen, halo, alkyl, aryl, alkylaryl, heteroaryl, cycloalkyl, or cycloheteroalkyl, optionally R⁴ is substituted at one or more positions with one or more of alkyl, alkoxy, haloalkyl, haloalkoxy, hydroxyl, halo, cyano, carboxyamido, carboxy, aryloxy, and heteroaryloxy;
X is O or NR⁵;
R⁵ is hydrogen, alkyl, or —C(O)R⁶;
R⁶ is aryl optionally substituted at one or more ring positions with one or more of alkyl, alkoxy, haloalkyl, haloalkoxy, aryl, hydroxyl, halo, cyano, amido, and carboxyl;
Y is alkylenyl, arylenyl, benzylenyl, heteroarylenyl, cycloalkylenyl, or cycloheteroalkylenyl;
L is a bond, or a linker selected from the group consisting of —(O(CH₂)₂)ₐ—, —(O(CH₂)₂)ₐ—NH—C(O)-Alk³-, —(((CH₂)₂)ₐ—C(O)—NH—((CH₂)₂—O)_{b}-Alk³-, —(O(CH₂)₂)ₐ—C(O)—NH-Alk³-C(O)—, —(((CH₂)₂)ₐ—C(O)—NH—((CH₂)₂—O)_{b}—, —(O(CH₂)₂)ₐ—C(O)—NH—((CH₂)₂—O)_{b}-Alk³-C(O)—, —(O(CH₂)₂)ₐ—C(O)—NH-Alk³-NH—C(O)—CH₂—, and —(((CH₂)₂)ₐ—C(O)—NH-Alk³;
a is an integer selected from 1-20;
b is an integer selected from 1-20;
Alk³ is straight-chain or branched alkylenyl;
M_{E3} is selected from the group consisting of

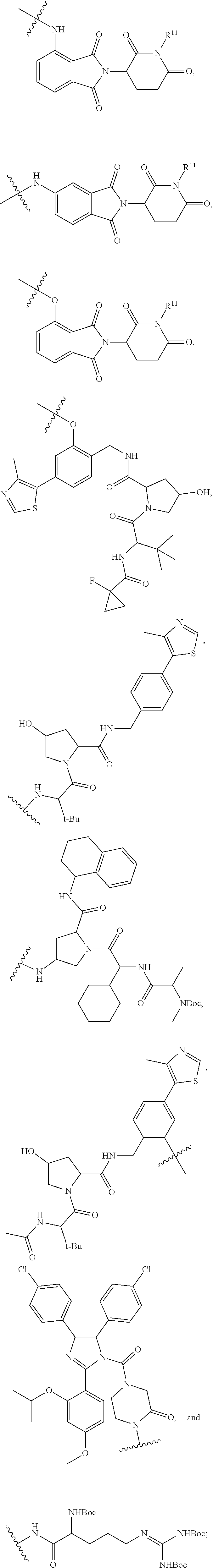

R⁷ is alkyl optionally substituted with one or more of amino or —S(O)₂R⁸;
R⁸ is alkyl or aryl, wherein R⁸ is optionally substituted at one or more positions with one or more of alkyl, alkoxy, haloalkyl, haloalkoxy, aryl, hydroxyl, halo, cyano, amido, and carboxyl; and
R⁹, R¹⁰, and R¹¹ are independently hydrogen or alkyl.

In some embodiments, the PROTACs are trifluoromethyl substituted 1-methyl-1H-pyrazole having a formula II(a):

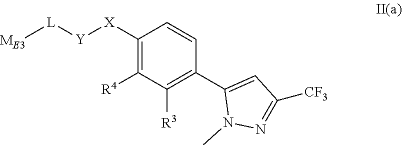

wherein
R³ is hydroxyl or —O—C(O)-alkyl;
R⁴ is hydrogen or phenyl substituted at one or more positions with one or more of haloalkyl or halo;
X is O or —N—C(O)R⁶;
R⁶ is aryl substituted at one or more ring positions with one or more of haloalkyl or halo;
Y is alkylenyl or benzylenyl;
L is a bond, or a linker selected from the group consisting of —(((CH₂)₂)ₐ—, —(O(CH₂)₂)ₐ—C(O)—NH—((CH₂)₂—O)_{b}-Alk³-, —(((CH₂)₂)ₐ—C(O)—NH-Alk³-C(O)—, —(((CH₂)₂)ₐ—C(O)—NH—((CH₂)₂—O)_{b}—, —(O(CH₂)₂)ₐ—C(O)—NH—((CH₂)₂—O)_{b}-Alk³-C(O)—, —(O(CH₂)₂)ₐ—C(O)—NH-Alk³-NH—C(O)—CH₂—, and —(O(CH₂)₂)ₐ—C(O)—NH-Alk³-; and
R⁸ is alkyl or aryl, wherein R⁸ is optionally substituted at one or more positions with one or more of alkyl.

In some embodiments of the disclosed PROTACs, the PROTACs have a structure of formula II(a) with R³ being hydroxyl, and R⁴ being phenyl substituted with trifluoromethyl and chloro.

In some embodiments of the disclosed PROTACs, the PROTACs have a structure of formula II(a) with X being —N—C(O)R⁶, and R⁶ being phenyl substituted with trifluoromethyl and chloro.

In some embodiments of the disclosed PROTACs, the PROTACs have a structure of formula II(a) with X being O, and Y being propylenyl or benzylenyl.

In some embodiments of the disclosed PROTACs, the PROTACs have an M_{E3} group selected from

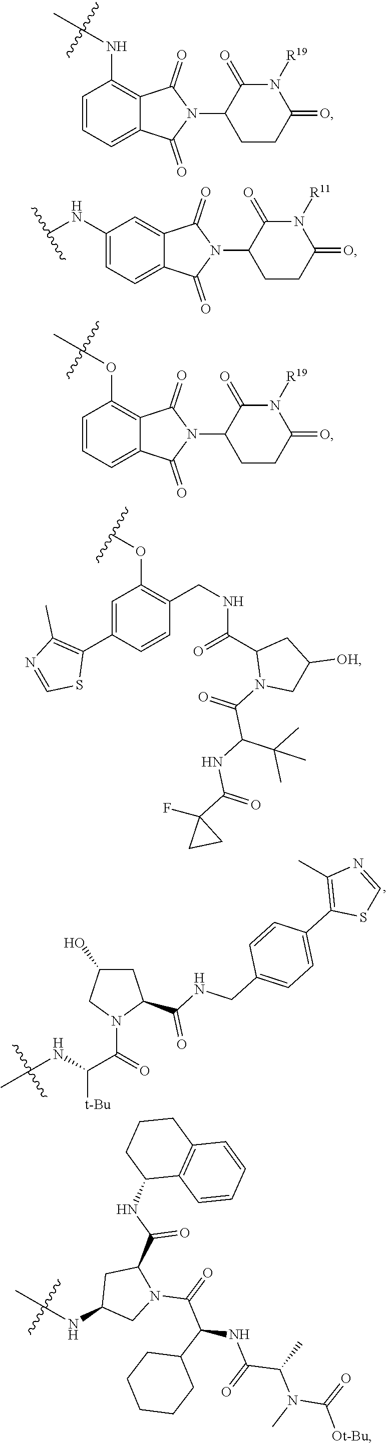

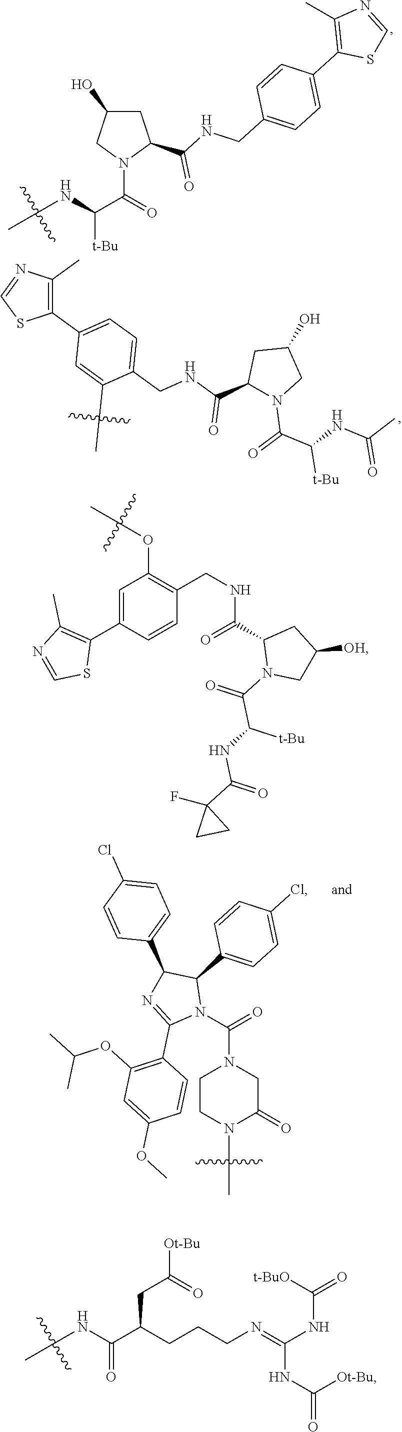

wherein R¹⁹ is hydrogen or methyl.

In some embodiments of the disclosed PROTACs, the PROTACs are selected from the group consisting of.

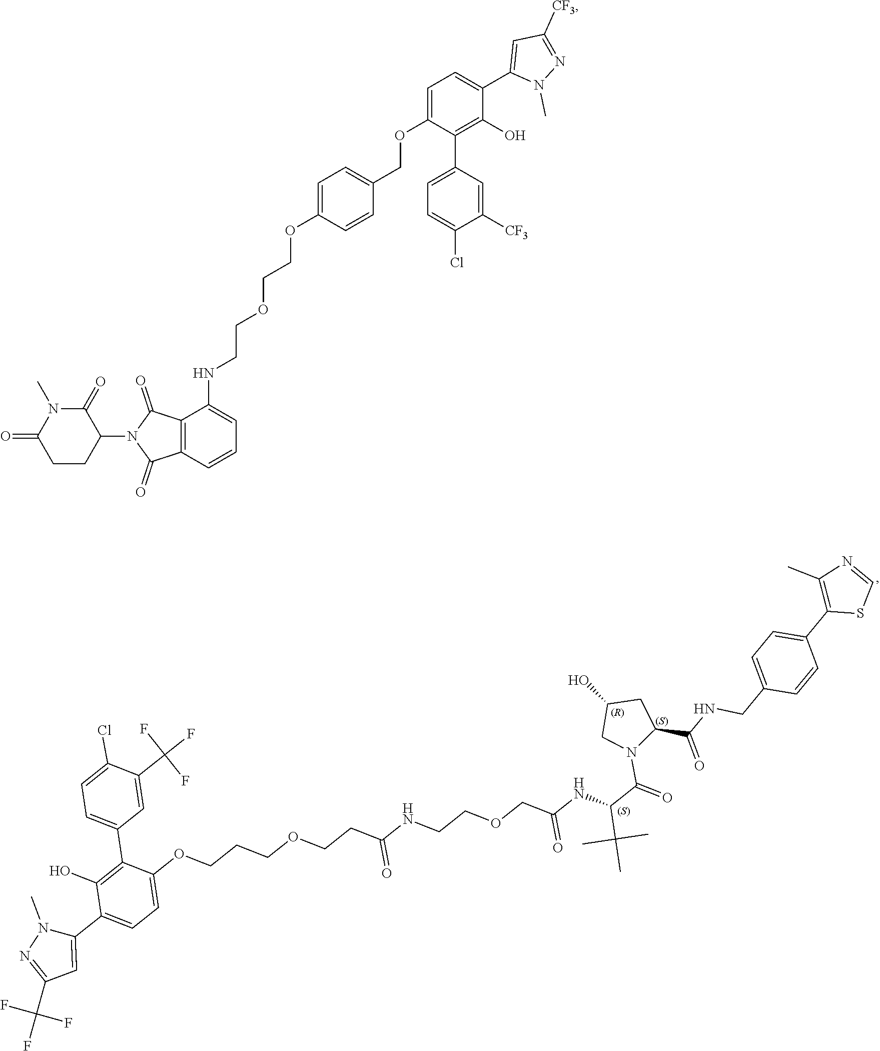

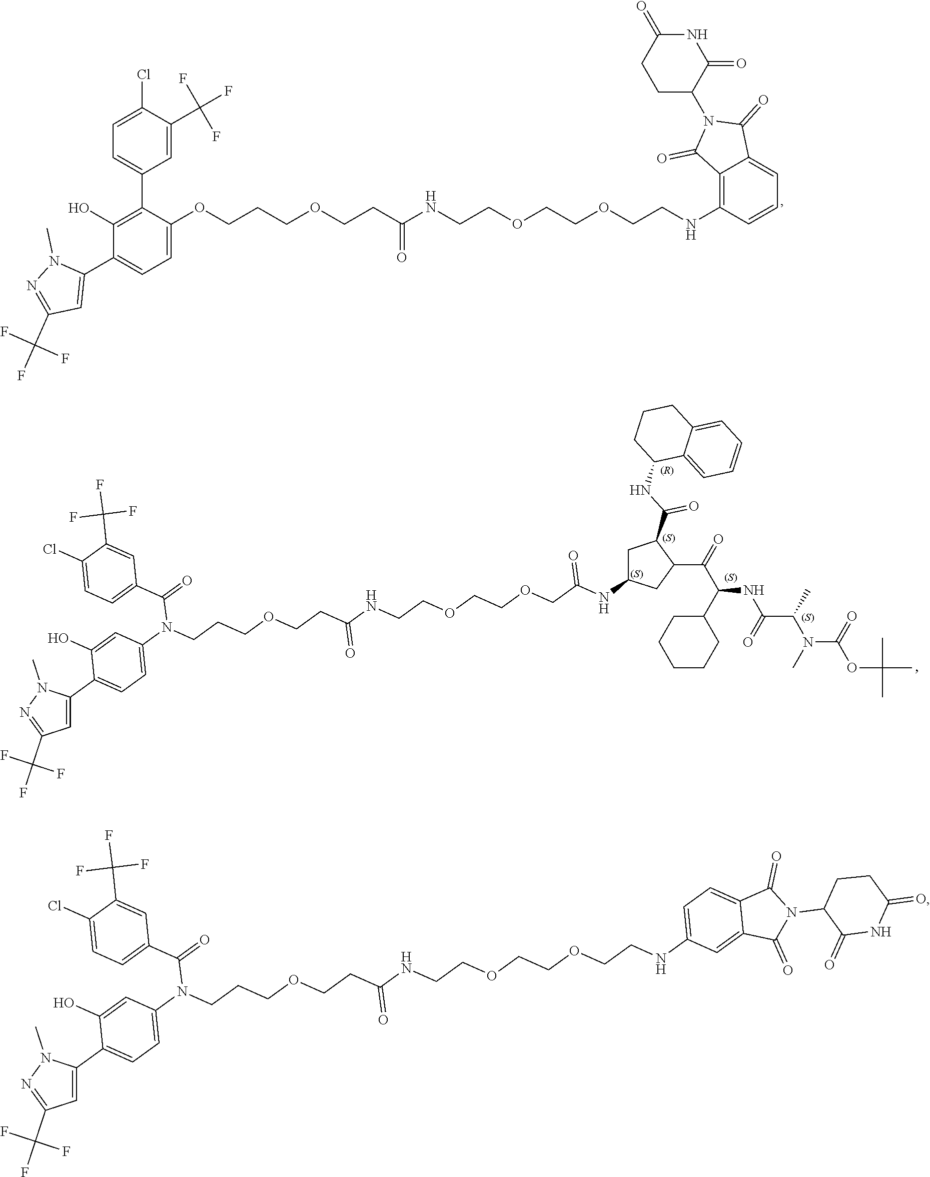

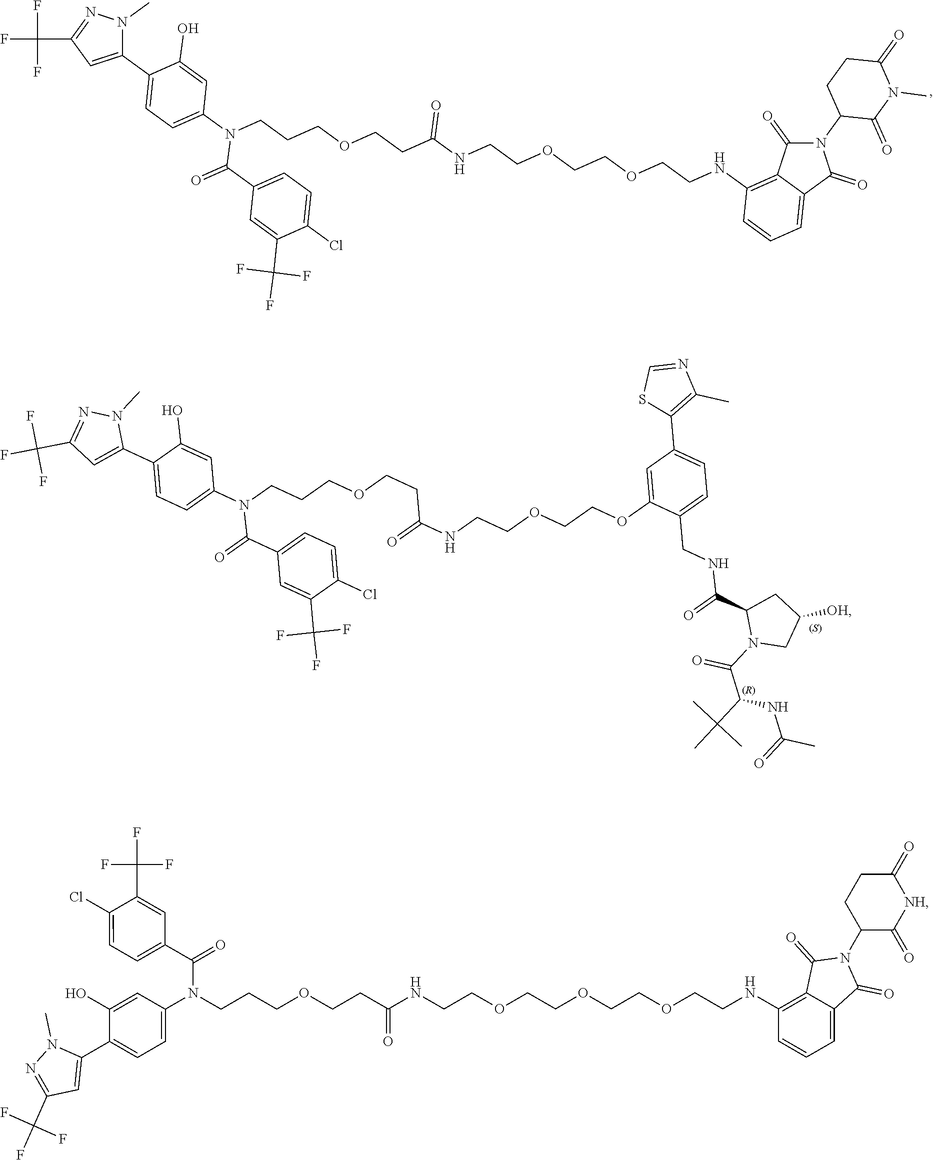

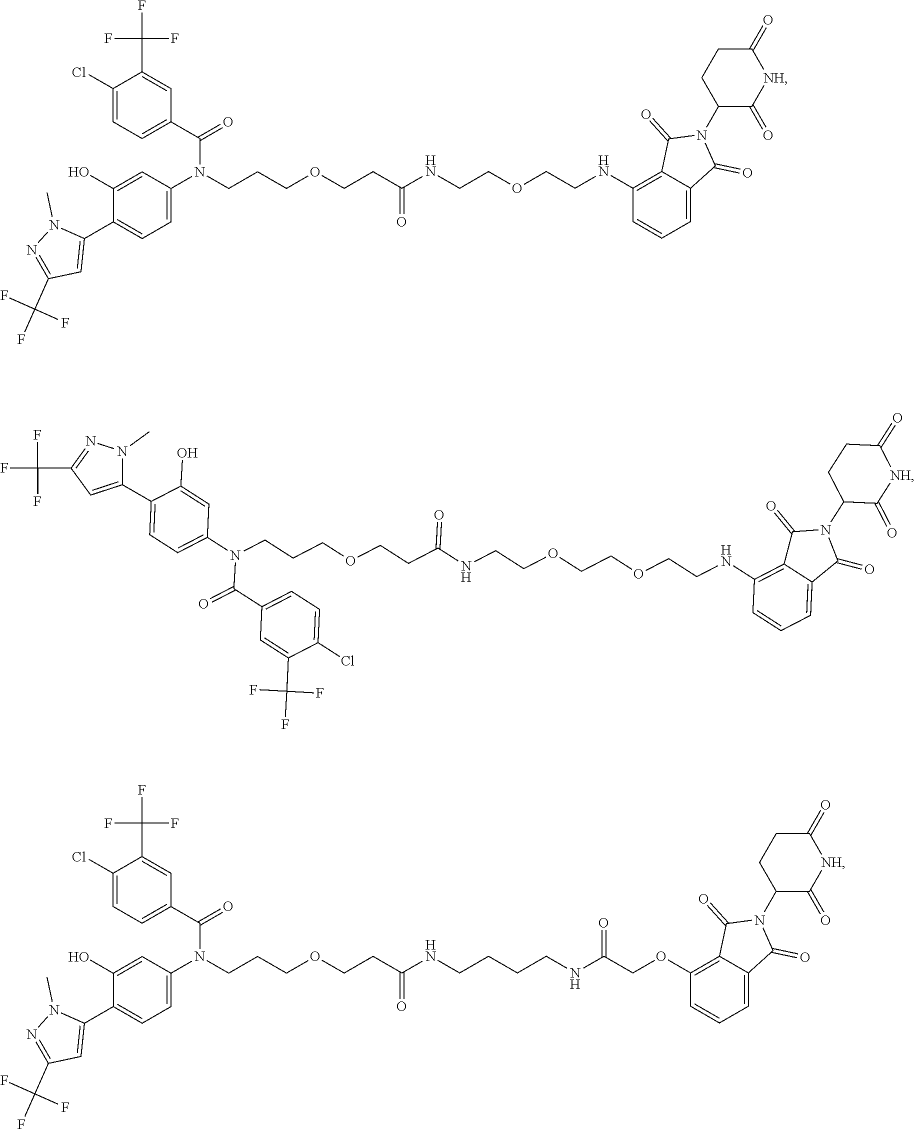 [SMILES: Cn1nc(C(F)(F)F)cc1-c1ccc(N(CCCOCCC(=O)NCCOCCNc2cccc3c2C(=O)N(C2CCC(=O)NC2=O)C3=O)C(=O)c2ccc(Cl)c(C(F)(F)F)c2)cc1O ; Cn1nc(C(F)(F)F)cc1-c1ccc(N(CCCOCCC(=O)NCCOCCOCCNc2cccc3c2C(=O)N(C2CCC(=O)NC2=O)C3=O)C(=O)c2ccc(Cl)c(C(F)(F)F)c2)cc1O ; Cn1nc(C(F)(F)F)cc1-c1ccc(N(CCCOCCC(=O)NCCCCNC(=O)COc2cccc3c2C(=O)N(C2CCC(=O)NC2=O)C3=O)C(=O)c2ccc(Cl)c(C(F)(F)F)c2)cc1O]

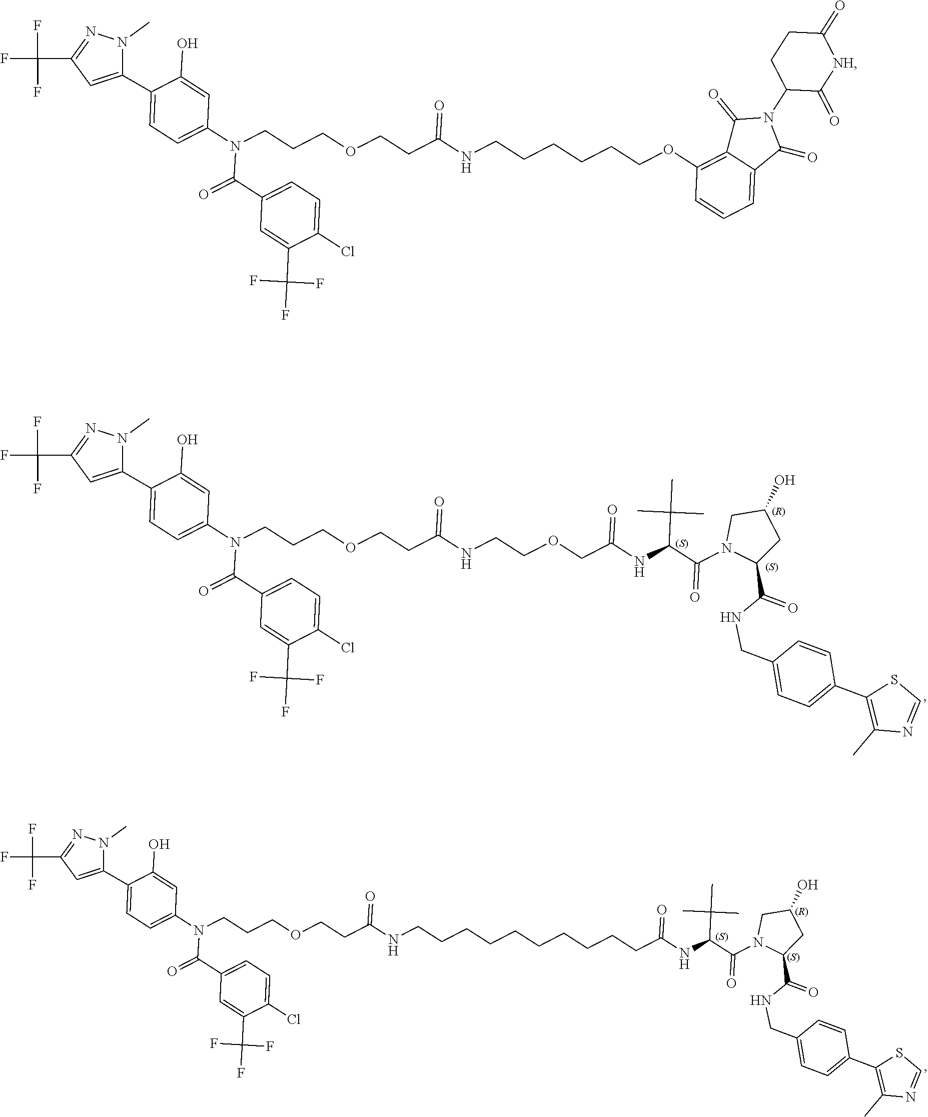

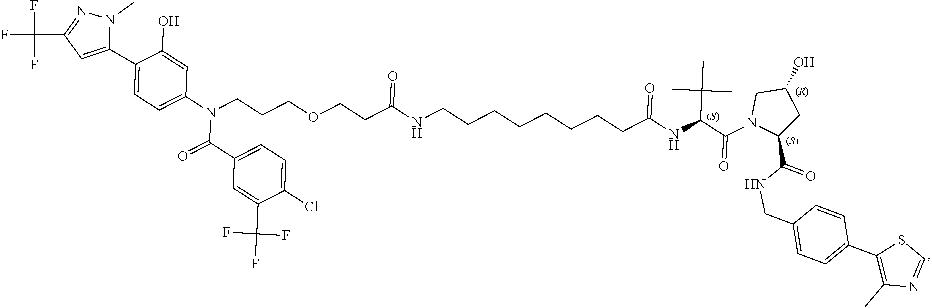

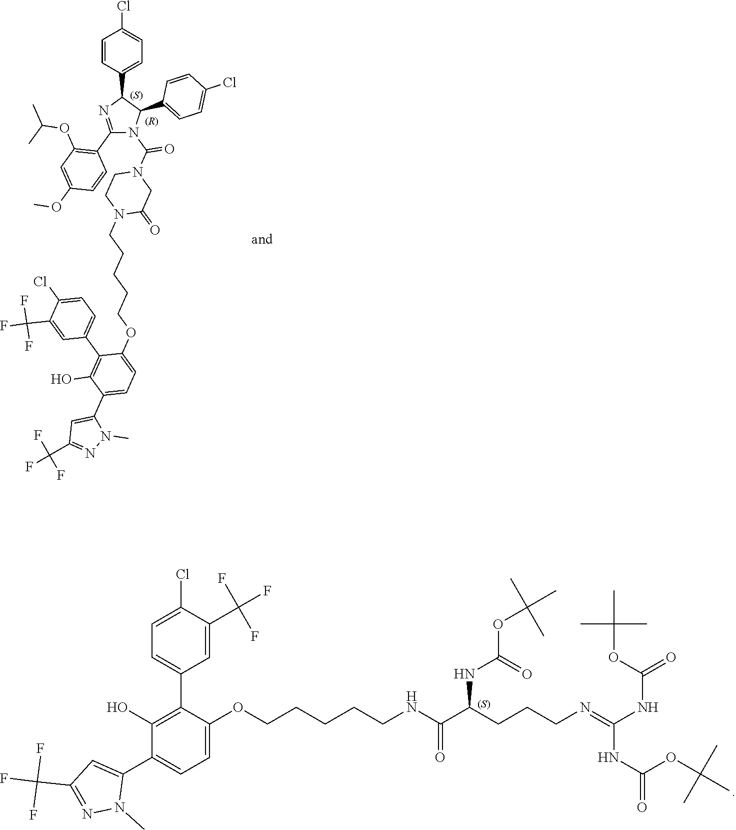

The formulae of the compounds disclosed herein should be interpreted as encompassing all possible stereoisomers, enantiomers, or epimers of the compounds unless the formulae indicate a specific stereoisomer, enantiomer, or epimer. The formulae of the compounds disclosed herein should be interpreted as encompassing salts, esters, amides, or solvates thereof of the compounds.

The disclosed compounds may exhibit one or more biological activities. The disclosed compounds may inhibit binding of the Myc/Max complex to DNA (e.g., in a DNA gel shifting assay). In some embodiments, the disclosed compounds inhibit binding of the Myc/Max complex to DNA by at least 50%, 60%, 70%, 80%, 90%, 95%, 96%, 97%, 98%, or 99% at a concentration of less than about 100 μM, 50 μM, 10 μM, 1 μM, 0.1 μM, 0.05 μM, 0.01 μM, 0.005 μM, 0.001 μM, or less. The disclosed compounds may not produce significant DNA damage (e.g., in an rH2AX staining assay at a concentration greater than about 0.001 μM, 0.005 μM, 0.01 μM, 0.1 μM, 1.0 μM, 10 μM, 100 μM, or higher). The disclosed compounds may inhibit the growth of cells that express c-Myc (preferably by at least 50%, 60%, 70%, 80%, 90%, 95%, 96%, 97%, 98%, or 99% at a concentration of less than about 100 μM, 50 μM, 10 μM, 1 μM, 0.1 μM, 0.05 μM, 0.01 μM, 0.005 μM, 0.001 μM, or less). The disclosed compounds may not inhibit the growth of cells that do not express c-Myc (preferably by not more than 50%, 40%, 30%, 20%, 10%, 5%, 4%, 3%, 2% or less at a concentration of greater than about 0.001 μM, 0.005 UM, 0.01 μM, 0.5 μM, 0.1 μM, 1.0 μM, 10 μM, and 100 μM or higher). Concentration ranges also are contemplated herein, for example, a concentration range bounded by end-point concentrations selected from 0.001 μM, 0.005 μM, 0.01 μM, 0.5 μM, 0.1 μM, 1.0 μM, 10 μM, and 100 μM.

The disclosed compounds may be effective in inhibiting cell proliferation of cancer cells, including cancer cells that express c-MYC and whose proliferation is inhibiting by inhibiting the biological activity of c-MYC. The disclosed compounds may be effective in inhibiting cell proliferation of one or more types of cancer cells including: multiple myeloma cells, such as MM.1S cells; leukemia cells, such as CCRF-CEM, HL-60 (TB), MOLT-4, RPMI-8226 and SR; non-small lung cancer cells, such as A549/ATCC, EKVX, HOP-62, HOP-92, NCI-H226, NCI-H23, NCI-H322M, NCI-H460 and NCI-H522, colon cancer cells, such as COLO 205, HCC-2998, HCT-116, HCT-15, HT29, KM12 and SW-620; CNS; SF-268, SF-295, SF-539, SNB-19, SNB-75 and U251; melanoma cancer cells, such as LOX IMVI, MALME-3M, M14, MDA-MB-435, SK-MEL-2, SK-MEL-28, SK-MEL-5, UACC-257 and UACC-62; ovarian cancer cells, such as IGR-OVI, OVCAR-3, OVCAR-4, OVCAR-5, OVCAR-8, NCI/ADR-RES and SK-OV-3; renal cancer cells, such as 786-0, A498, ACHN, CAKI-1, RXF 393, SN12C, TK-10 and UO-31; prostate cancer cells, such as DU-145 and PC-3; and breast cancer cells, such as MCF7, MDA-MB-231/ATCC, MDA-MB-468, HS 578T, BT-549 and T-47D.

Cell proliferation and inhibition thereof by the presently disclosed compounds may be assessed by cell viability methods disclosed in the art including colorimetric assays that utilize dyes such as MTT, XTT, and MTS to assess cell viability. Preferably, the disclosed compounds have an IC₅₀ of less than about 10 μM, 5 μM, 1 μM, 0.5 μM, 0.01 μM, 0.005 μM, 0.001 μM or lower in the selected assay.

The disclosed compounds may be formulated as anti-cancer therapeutics, including hematologic malignancies, breast, lung, pancreas and prostate malignancies. The disclosed compounds also may be formulated as anti-inflammation therapeutics.

The compounds utilized in the methods disclosed herein may be formulated as pharmaceutical compositions that include: (a) a therapeutically effective amount of one or more compounds as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 UM (e.g., 0.1 μM-1.0 μM).

The disclosed compounds and pharmaceutical compositions comprising the disclosed compounds may be administered in methods of treating a subject in need thereof. For example, in the methods of treatment a subject in need thereof may include a subject having a cell proliferative disease, disorder, or condition such as cancer (e.g., cancers such as multiple myeloma, leukemia, non-small cell lung cancer, colon cancer, cancer of the central nervous system, melanoma, ovarian cancer, renal cancer, prostate cancer, and breast cancer).

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as end-points any of these disclosed doses (e.g., 2.5 mg-200 mg).

In some embodiments, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as end-points any of these disclosed dose levels (e.g., 500-2000 ng/kg body weight of the subject).

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSoly SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are preferably applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formations may be presented in unit-dose or multidose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

The disclosed compounds or pharmaceutical compositions comprising the disclosed compounds may be administered in methods of treatment. For example, the disclosed compounds or pharmaceutical compositions comprising the disclosed compounds may be administered in methods of treating cell proliferative diseases and disorders. Cell proliferative diseases and disorders treated by the disclosed methods may include, but are not limited to, cancers selected from the group consisting of multiple myeloma, leukemia, non-small cell lung cancer, colon cancer, cancer of the central nervous system, melanoma, ovarian cancer, renal cancer, prostate cancer, and breast cancer.

Optionally, the disclosed compounds or pharmaceutical compositions comprising the disclosed compounds may be administered with additional therapeutic agents, optionally in combination, in order to treat cell proliferative diseases and disorders. In some embodiments of the disclosed methods, one or more additional therapeutic agents are administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical composition is formulated to comprise the disclosed compounds and further to comprise one or more additional therapeutic agents, for example, one or more additional therapeutic agents for treating cell proliferative diseases and disorders.

In some embodiments, additional therapeutic agents may include, but are not limited to, therapeutic agents for treating leukemias and lymphomas, such as acute myeloid leukemia (AML), acute lymphocytic leukemia (ALL), chronic myelogenous leukemia (CML), and non-Hodgkin's lymphoma.

In some embodiments, additional therapeutic agents may include, but are not limited to, antimetabolite antineoplastic agents that inhibit the synthesis of DNA. Suitable antimetabolite antineoplastic agents that inhibit the synthesis of DNA may include, but are not limited to, nucleoside and/or nucleotide derivatives. Suitable nucleoside and/or nucleotide derivatives may include, but are not limited to cytosine arabinoside (ara-C), otherwise called cytarabine.

### Examples

The following Examples are illustrative and are not intended to limit the scope of the claimed subject matter.

### Example 1

### Synthesis of A4BC1R1 (NUCC-0226301), A4BC1R3 (NUCC-0226276), A4BC1R4 (NUCC-0226302), A4BC1R⁵ (NUCC-0226277) and A4BC1_2Ts (NUCC-0226263)

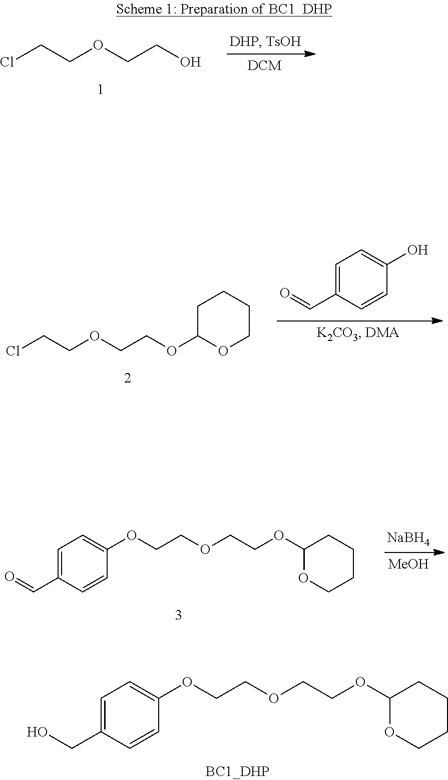

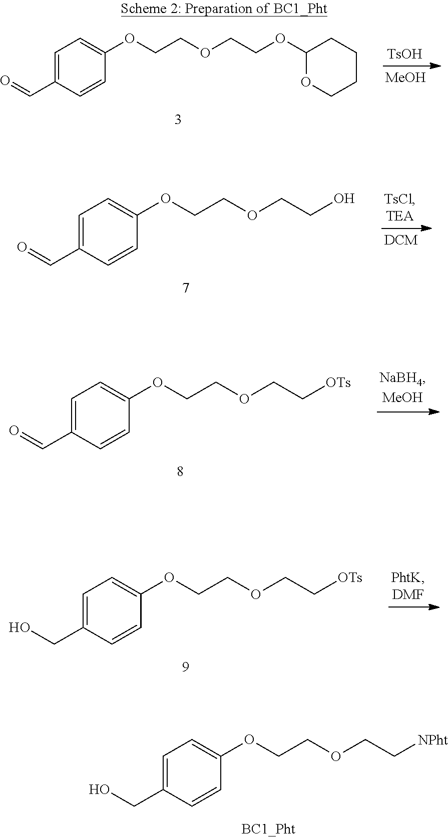

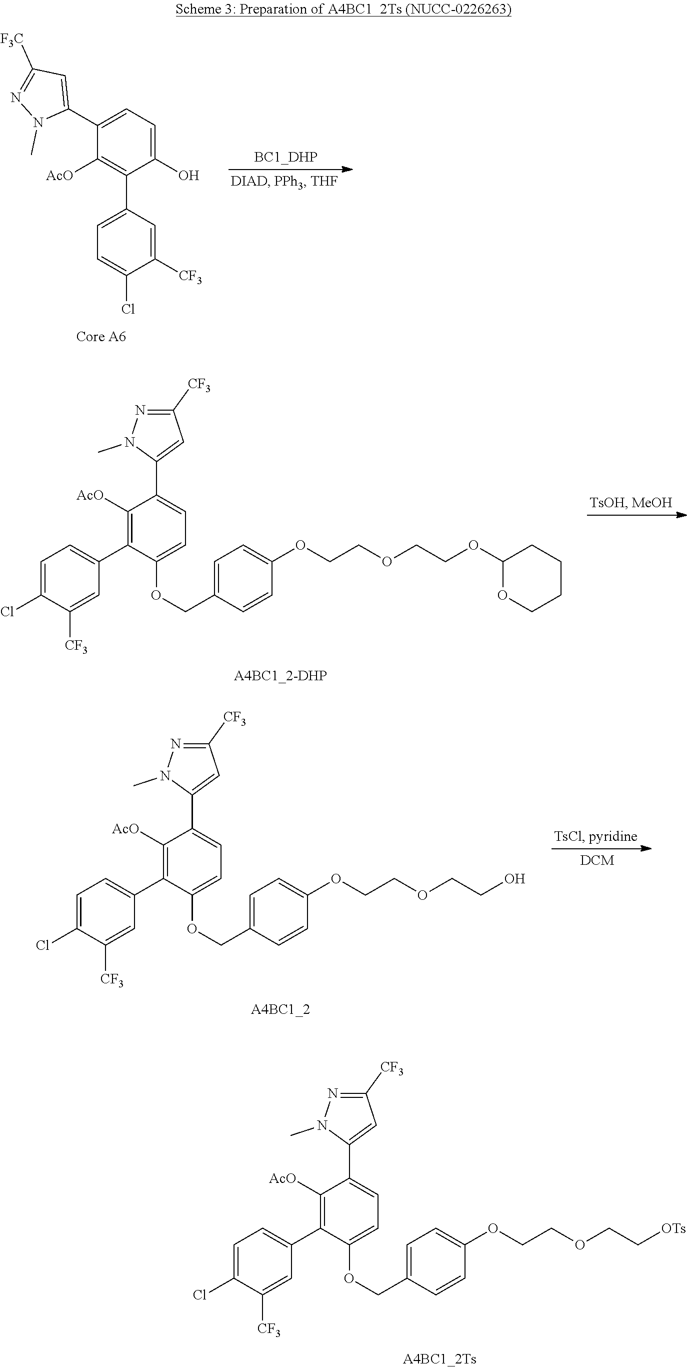 [SMILES: CC(=O)Oc1c(-c2cc(C(F)(F)F)nn2C)ccc(O)c1-c1ccc(Cl)c(C(F)(F)F)c1 ; CC(=O)Oc1c(-c2cc(C(F)(F)F)nn2C)ccc(OCc2ccc(OCCOCCOC3CCCCO3)cc2)c1-c1ccc(Cl)c(C(F)(F)F)c1 ; CC(=O)Oc1c(-c2cc(C(F)(F)F)nn2C)ccc(OCc2ccc(OCCOCCO)cc2)c1-c1ccc(Cl)c(C(F)(F)F)c1 ; CC(=O)Oc1c(-c2cc(C(F)(F)F)nn2C)ccc(OCc2ccc(OCCOCCOS(=O)(=O)c3ccc(C)cc3)cc2)c1-c1ccc(Cl)c(C(F)(F)F)c1]

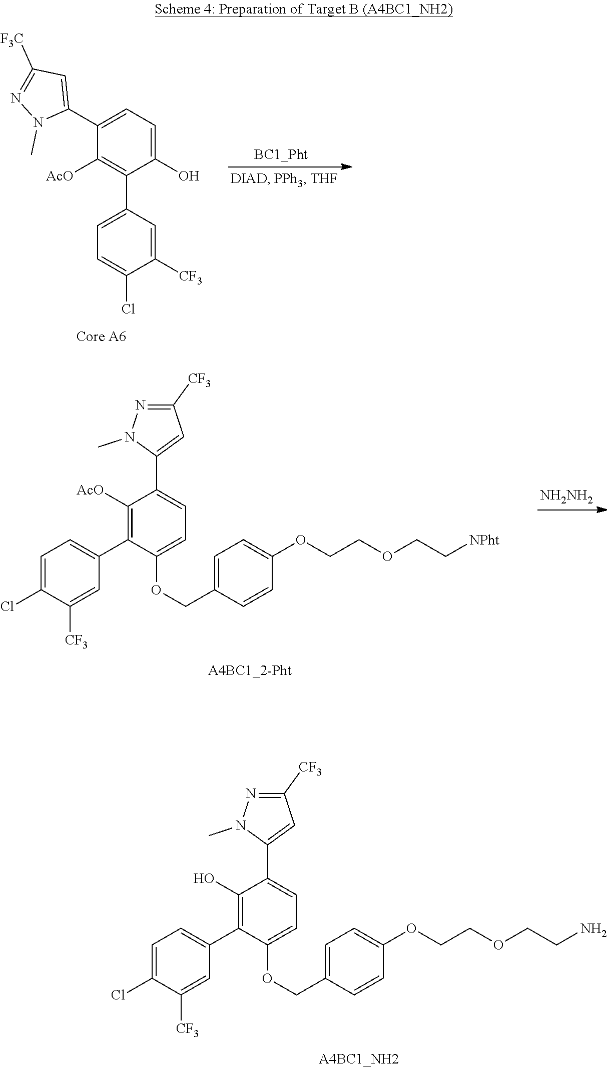

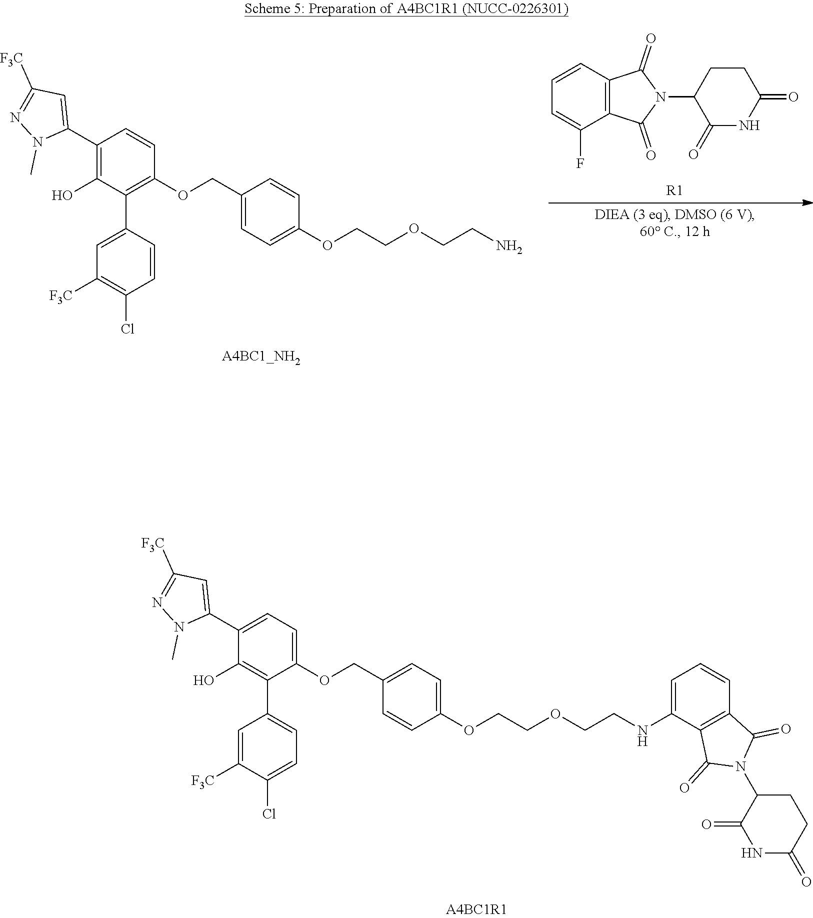

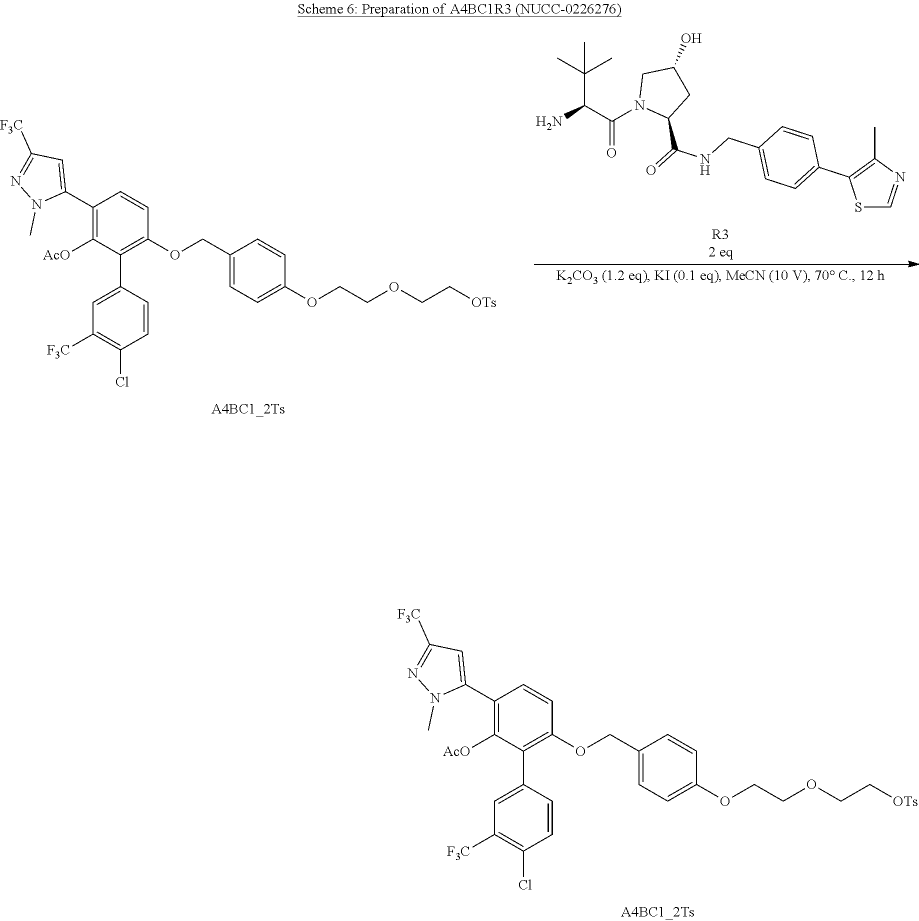 [SMILES: CC(=O)Oc1c(-c2cc(C(F)(F)F)nn2C)ccc(OCc2ccc(OCCOCCOS(=O)(=O)c3ccc(C)cc3)cc2)c1-c1ccc(Cl)c(C(F)(F)F)c1 ; Cc1ncsc1-c1ccc(CNC(=O)[C@@H]2C[C@@H](O)CN2C(=O)[C@@H](N)C(C)(C)C)cc1 ; CC(=O)Oc1c(-c2cc(C(F)(F)F)nn2C)ccc(OCc2ccc(OCCOCCOS(=O)(=O)c3ccc(C)cc3)cc2)c1-c1ccc(Cl)c(C(F)(F)F)c1]

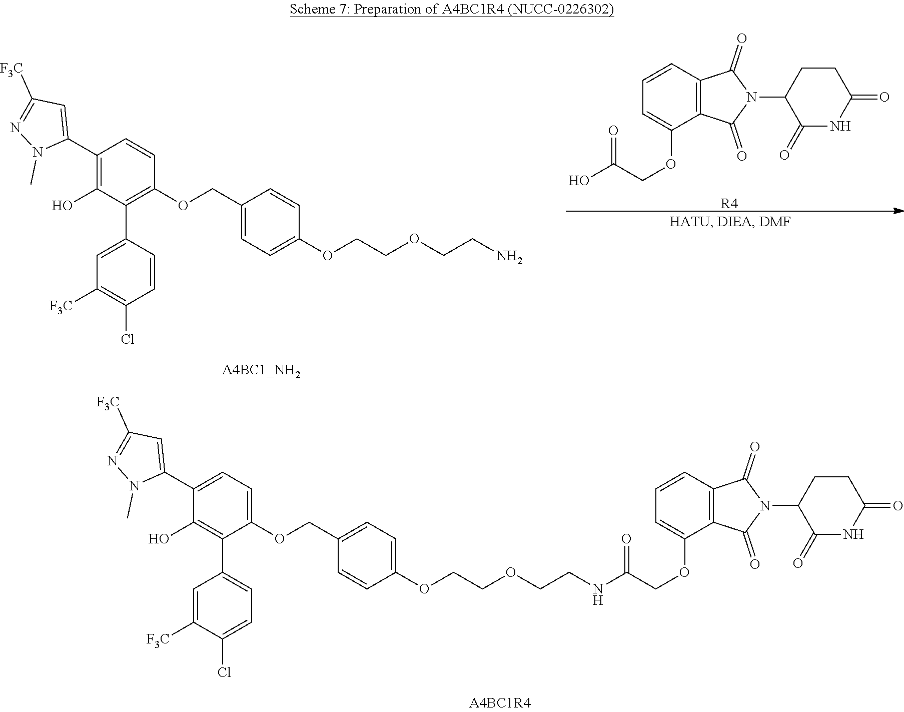

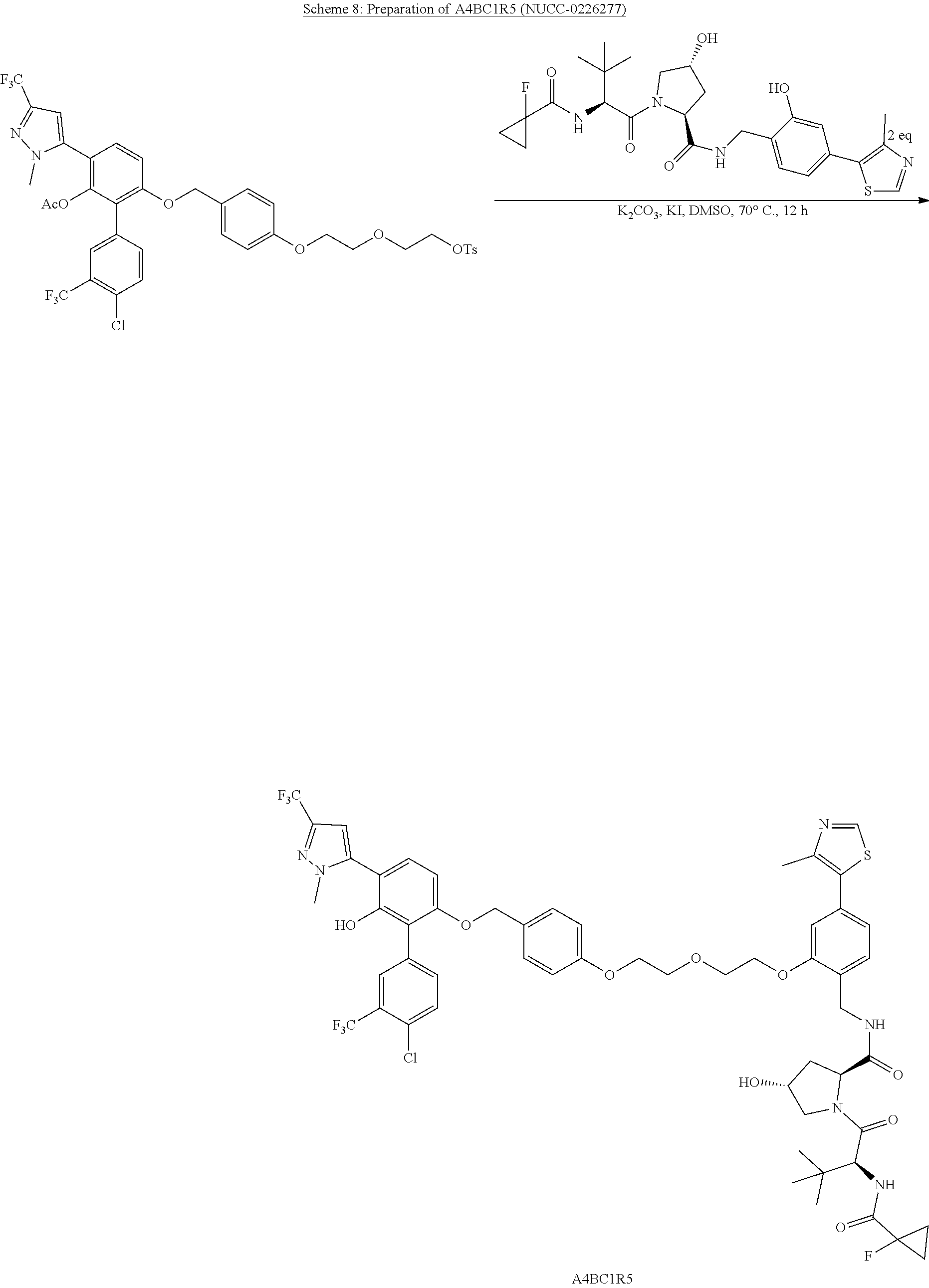

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual slovent peak (CDCl₃, 1H δ=7.27; CD₃OD, 1H δ=3.31; DMSO-d₆, 1H δ=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

### Method 1: 5_95AB_6min-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

### Method 2:5_95CD_6min-220-254-ELSD

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄CO₃, mobile phase B was acetonitrile. The column used for chromatography was a Xbridge Shield RP18 2.1*50 mm column (5 um particles). Detection methods are diode array (DAD) and evaporative light scattering (ELSD) detection as well as positive electrospray ionization. MS range was 100-1000.

### Method 3:5-95AB_2_min

LC/MS (The column used for chromatography was a Kinetex Sum EVO C18 100A 2.1*30 mm. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

### Method 4: 5_95CD_6min_MS1500-220-254-ELSD

LC/MS (The gradient was 5% B in 0.40 min and 5-95% B at 0.40-3.40 min hold on 95% B for 0.45 min, and then 95-5% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (Sum particles). Detection methods are diode array (DAD) and evaporative light scattering (ELSD) detection. MS mode was positive electrospray ionization. MS range was 100-1500.

### Method 5: 5_95AB_6min_MS1500-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Luna C18 50*2.0 mm column (Sum particles). Detection methods are diode array (DAD) and evaporative light scattering (ELSD) detection. MS mode was positive electrospray ionization. MS range was 100-1500.

### Experimentals for Largest Scale Run

### General Procedure for Preparation of Cpd 2-ET37412-62

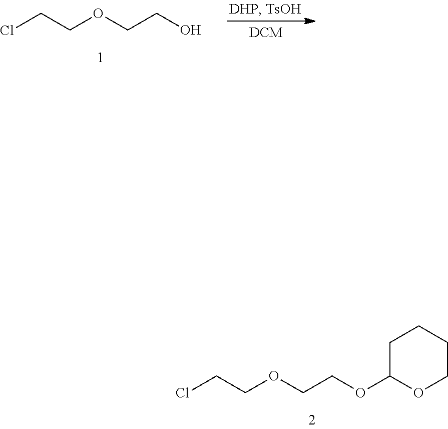 [SMILES: OCCOCCCl ; ClCCOCCOC1CCCCO1]

To a solution of DHP (2.3 g, 27.29 mmol, 1.7 eq) in DCM (20 mL) was added Cpd 1 (2 g, 16.06 mmol, 1 eq), TsOH (276 mg, 1.61 mmol, 0.1 eq) in one portion at 25° C. under N₂. The mixture was stirred at 25° C. for 12 h. TLC showed the reaction was completed. The reaction was concentrated to give a residue which was purified by silica gel chromatography (column height: 250 mm, diameter: 100 mm, 100-200 mesh silica gel, Petroleum ether/Ethyl acetate=1/0, 0/1) to afford Cpd 2 (2 g, 59.69% yield) as colorless oil.

¹H NMR (ET37412-62-1, 400 MHz, CHLOROFORM-d) δ 1.47-1.66 (m, 6H), 1.70-1.77 (m, 1H), 1.80-1.90 (m, 1H), 3.47-3.55 (m, 1H), 3.58-3.67 (m, 3H), 3.69-3.74 (m, 2H), 3.76-3.81 (m, 2H), 3.84-3.92 (m, 2H), 4.65 (t, J=3.64 Hz, 1H)

### General Procedure for Preparation of Cpd 3-ET37412

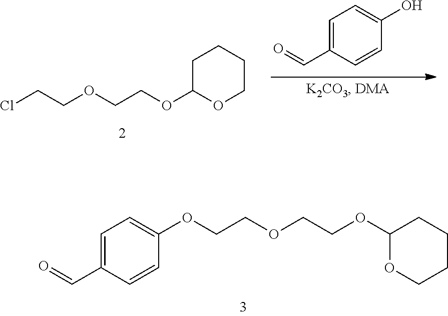

To a solution of 4-hydroxybenzaldehyde (900 mg, 7.37 mmol, 1 eq) in DMA (10 mL) was added Cpd 2 (1.85 g, 8.84 mmol, 1.2 eq), K₂CO₃ (2.04 g, 14.74 mmol, 2 eq) in one portion at 25° C. under N₂. The mixture was stirred at 90° C. for 12 hrs. LCMS showed the reaction was completed. The reaction mixture was poured into ice-water (10 mL). The aqueous phase was extracted with ethyl acetate (10 mL×2). The combined organic phase was washed with brine (10 mL), dried with anhydrous Na₂SO₄, filtered and concentrated in vacuum to give Cpd 3 (1.5 g, 69.15% yield) was obtained as colorless oil.

$$LCMS ( ESI + ) \text{:} RT = 0.824 min , m / z 211. {( M - DHP )}^{+} .$$

### 5-95AB_2_min

LC/MS (The column used for chromatography was a Kinetex 5 μm EVO C18 100A 2.1*30 mm. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

### General Procedure for Preparation of BC1_DHP-ET37412-71

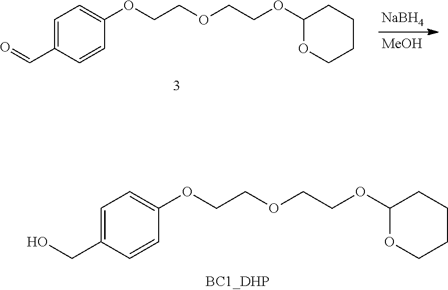

To a solution of Cpd 3 (1.4 g, 23.78 mmol, 1 eq) in MeOH (14 mL) was added NaBH₄ (269 mg, 7.13 mmol, 1.5 eq) in one portion at 25° C. under N₂. The mixture was stirred at 25° C. for 12 h. The mixture was poured into ice-water (10 mL). The aqueous phase was extracted with ethyl acetate (10 mL×2). The combined organic phase was washed with brine (10 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1/0, 0/1) to give BC1_DHP (1 g, 71% yield) as colorless oil.

¹H NMR (ET37412-71-1 400 MHz, CHLOROFORM-d) δ 1.48-1.65 (m, 5H), 1.70-1.78 (m, 1H), 1.79-1.91 (m, 1H), 3.48-3.57 (m, 1H), 3.62-3.69 (m, 1H), 3.77 (t. J=4.83 Hz, 2H), 3.84-3.95 (m, 4H), 4.08-4.24 (m, 2H), 4.59-4.71 (m, 3H), 6.93 (d, J=8.56 Hz, 2H), 7.19-7.37 (m, 3H)

### General Procedure for Preparation of Cpd 7-ET43259-3

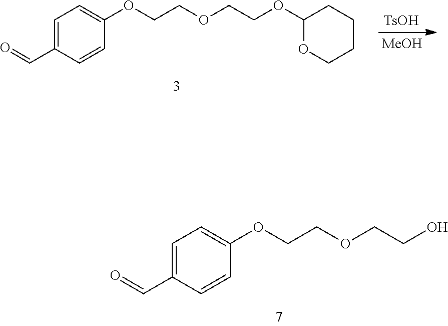 [SMILES: "MeOH" ; O=Cc1ccc(OCCOCCOC2CCCCO2)cc1 ; "TsOH" ; O=Cc1ccc(OCCOCCO)cc1]

To a solution of Cpd 3 (2 g, 6.79 mmol, 1 eq) in MeOH (20 mL) was added TsOH (2.34 g, 13.59 mmol, 2 eq) in one portion at 25° C. under N₂. The mixture was stirred at 25° C. for 12 h. The reaction was concentrated to Cpd 7 (1.2 g, 84% yield) as colorless oil.

¹H NMR (ET37412-92-1, 400 MHz, CHLOROFORM-d) δ 3.63-3.71 (m, 1H), 3.63-3.71 (m, 1H), 3.78 (br d, J=3.79 Hz, 2H), 3.88-3.97 (m, 2H), 4.19-4.26 (m, 2H), 7.03 (br d, J=8.44 Hz, 2H), 7.70-7.96 (m, 2H), 9.77-9.99 (m, 1H)

### General Procedure for Preparation of Cpd 8-ET37412-103

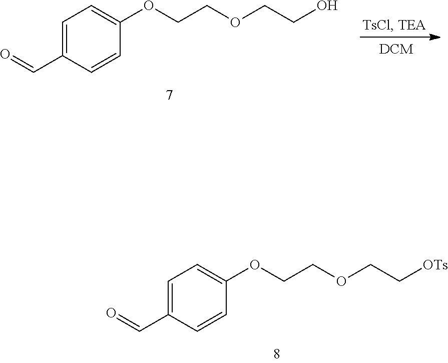 [SMILES: O=Cc1ccc(OCCOCCO)cc1 ; Cc1ccc(S(=O)(=O)OCCOCCOc2ccc(C=O)cc2)cc1]

To a solution of Cpd 7 (1 g, 4.76 mmol, 1 eq) in DCM (10 mL) was added TEA (1.93 g, 19.03 mmol, 4 eq), TosCl (1.36 g, 7.14 mmol, 1.5 eq) in one portion at 25° C. under N₂. The mixture was stirred at 25° C. for 12 hrs. The residue was poured into ice-water (10 mL). The aqueous phase was extracted with DCM (10 mL). The organic phase was washed with brine (5 mL), dried with anhydrous Na₂SO₄, filtered and concentrated in vacuum to give Cpd 8 (1 g, 58% yield) was obtained as colorless oil.

$$LCMS ( ESI + ) : RT = 0.931 min , m / z 365. {( M + 1 )}^{+} .$$

### 5-95AB_2min

LC/MS (The column used for chromatography was a Kinetex Sum EVO C18 100A 2.1*30 mm. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 ml/min.

### General Procedure for Preparation of Cpd 9-ET37412-105

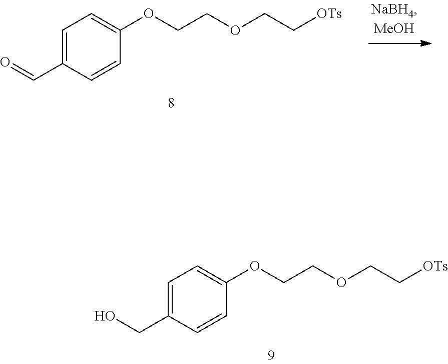 [SMILES: Cc1ccc(S(=O)(=O)OCCOCCOc2ccc(C=O)cc2)cc1 ; Cc1ccc(S(=O)(=O)OCCOCCOc2ccc(CO)cc2)cc1]

To a solution of Cpd 8 (905.77 mg, 2.49 mmol, 1 eq) in MeOH (2 mL) was added NaBH₄ (103.43 mg, 2.73 mmol, 1.1 eq) in one portion at 25° C. under N₂. The mixture was stirred at 25° C. for 1 h. LCMS showed the reaction was completed. The residue was poured into ice-water (10 mL). The aqueous phase was extracted with ethyl acetate (20 mL). The combined organic phase was washed with brine (10 mL), dried with anhydrous Na₂SO₄, filtered and concentrated to give Cpd 9 (0.3 g, 33% yield) was obtained as white solid.

¹H NMR (ET37412-105-1, 400 MHz, CHLOROFORM-d) δ 2.43 (s, 3H), 2.66 (dd, J=6.72, 2.57 Hz, 1H), 3.70-3.81 (m, 4H), 4.00-4.23 (m, 5H), 4.63 (s, 2H), 6.85-6.92 (m, 2H), 7.28-7.41 (m, 5H), 7.80 (d, J=8.31 Hz, 2H)

### General Procedure for Preparation of BC1_Pht ET37412-115

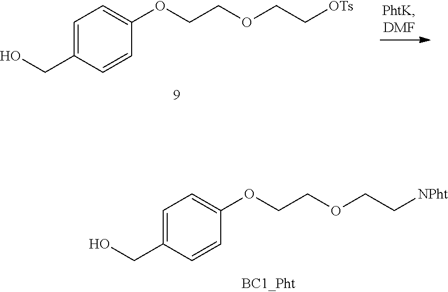

To a solution of Cpd 9 (230 mg, 627.68 μmol, 1 eq) in DMF (1 mL) was added (1,3-dioxoisoindolin-2-yl)-potassium (127.89 mg, 690.45 μmol, 1.1 eq) at 0° C. under N₂. The mixture was stirred at 90° C. for 12 h. LCMS showed the reaction was completed. The residue was poured into ice-water (2 mL). The aqueous phase was extracted with ethyl acetate (5 mL). The combined organic phase was washed with brine (5 mL), dried with anhydrous Na₂SO₄, filtered and concentrated to give the residue which was purified by silica gel chromatography (column height: 250 mm, diameter: 100 mm, 100-200 mesh silica gel, Petroleum ether/Ethyl acetate=1/0, 0/1) to give BC1_Pht (100 mg, 47% yield) as yellow solid.

¹H NMR (ET37412-115-1, 400 MHz, CHLOROFORM-d) δ 3.79-3.87 (m, 5H), 3.89-3.95 (m, 2H), 4.03-4.09 (m, 2H), 4.59 (s, 2H), 6.77-6.83 (m, 2H), 7.22 (br d, J=7.06 Hz, 2H), 7.64-7.74 (m, 2H), 7.78-7.87 (m, 2H)

### General Procedure for Preparation of A4BC1_2-DHP-ET37412-96

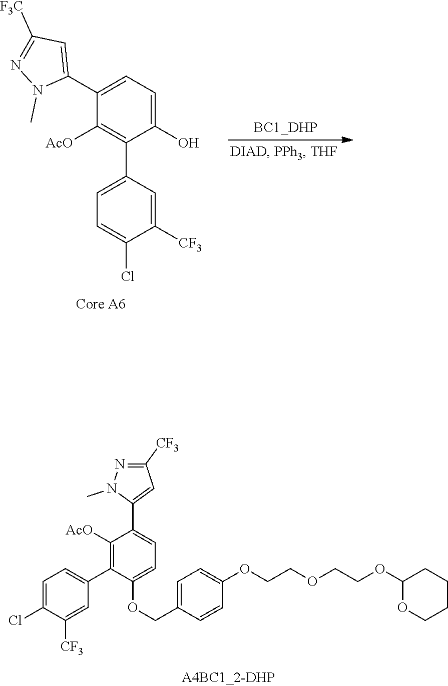 [SMILES: CC(=O)Oc1c(-c2cc(C(F)(F)F)nn2C)ccc(O)c1-c1ccc(Cl)c(C(F)(F)F)c1 ; CC(=O)Oc1c(-c2cc(C(F)(F)F)nn2C)ccc(OCc2ccc(OCCOCCOC3CCCCO3)cc2)c1-c1ccc(Cl)c(C(F)(F)F)c1]

To a solution of Core A6 (1 g, 2.09 mmol, 1 eq) and BC1_DHP (928.5 mg, 3.13 mmol, 1.5 eq) in THF (10 mL) was added PPh3 (2.47 g, 9.4 mmol, 4.5 eq) and DEAD (1.9 g, 9.4 mmol, 4.5 eq) at 0° C. under N₂. The mixture is stirred at 25° C. for 12 h. The mixture was added ice-water (20 mL). The aqueous phase was extracted with ethyl acetate (30 mL). The combined organic phase was washed with brine (20 mL), dried with anhydrous Na₂SO₄, filtered and concentrated in vacuum. The residue was purified by Prep-TLC to give A4BC1_2DHP (800 mg, 50% yield) as yellow oil.

$$LCMS ( ESI + ) : RT = 0.976 min , m / z 779.3 {( M + 23 )}^{+} .$$

### 5-95AB_2_min

LC/MS (The column used for chromatography was a Kinetex 5 μm EVO C18 100A 2.1*30 mm. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

### General Procedure for Preparation of A4BC1_2-ET37412-106

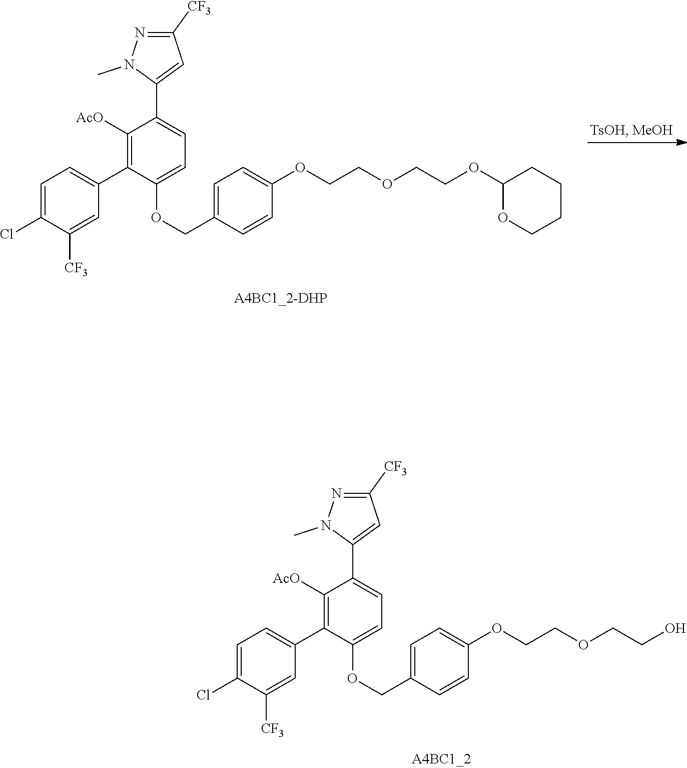 [SMILES: CC(=O)Oc1c(-c2cc(C(F)(F)F)nn2C)ccc(OCc2ccc(OCCOCCOC3CCCCO3)cc2)c1-c1ccc(Cl)c(C(F)(F)F)c1 ; CC(=O)Oc1c(-c2cc(C(F)(F)F)nn2C)ccc(OCc2ccc(OCCOCCO)cc2)c1-c1ccc(Cl)c(C(F)(F)F)c1]

To a solution of A4BC1_2DHP (800 mg, 1.06 mmol, 1 eq) in MeOH (10 mL) was added TsOH (363 mg, 2.11 mmol, 2 eq) in one portion at 25° C. under N₂. The mixture was stirred at 25° C. for 12 h. TLC showed the reaction was completed. The mixture was concentrated to give A4BC1_2 (500 mg, 794 μmol, 70% yield) as white oil. The residue was used to next step without any purification.

### General Procedure for Preparation of A4BC1_2Ts (NUCC-0226263)-ET37412-104

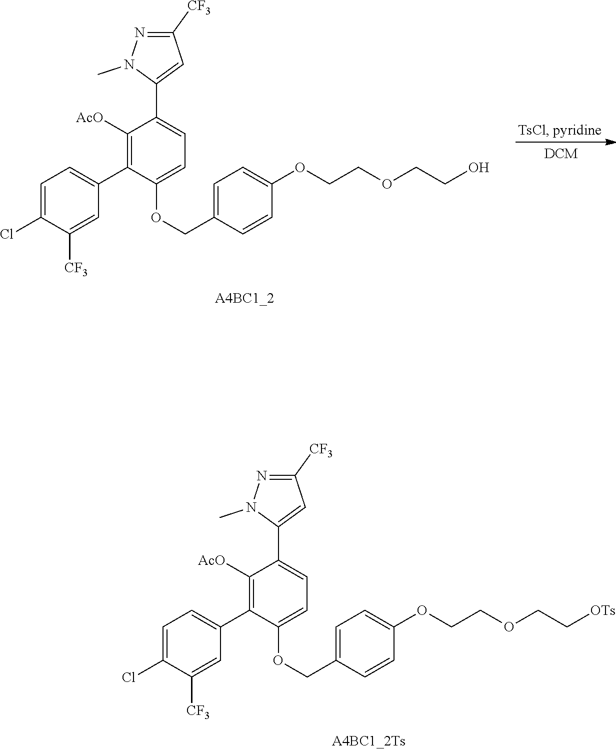 [SMILES: CC(=O)Oc1c(-c2cc(C(F)(F)F)nn2C)ccc(OCc2ccc(OCCOCCO)cc2)c1-c1ccc(Cl)c(C(F)(F)F)c1 ; CC(=O)Oc1c(-c2cc(C(F)(F)F)nn2C)ccc(OCc2ccc(OCCOCCOS(=O)(=O)c3ccc(C)cc3)cc2)c1-c1ccc(Cl)c(C(F)(F)F)c1]

To a solution of A4BC1_2 (500 mg, 742 μmol, 1 eq) in DCM (5 mL) was added Py (587 mg, 7.43 mmol, 10 eq) and TosCl (1.42 g, 7.43 mmol, 10 eq) in one portion at 25° C. under N₂. The mixture was stirred at 25° C. for 12 h. LCMS showed the reaction was completed. The mixture was added ice-water (10 mL). The aqueous phase was extracted with DCM (20 mL). The combined organic phase was washed with brine (20 mL), dried with anhydrous Na₂SO₄, filtered and concentrated to give the residue which was purified by silica gel chromatography (column height: 250 mm, diameter: 100 mm, 100-200 mesh silica gel, Petroleum ether/Ethyl acetate=1/0, 0/1) to give A4BC1_2 Ts (320 mg, 52% yield) as white solid.

$$LCMS ( ESI + ) : RT = 0.969 min , m / z 827.2 {( M + 1 )}^{+} .$$

### 5-95AB_2_min

LC/MS (The column used for chromatography was a Kinetex Sum EVO C18 100A 2.1*30 mm. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

### General Procedure for Preparation of A4BC1_2Pht-ET37412-117

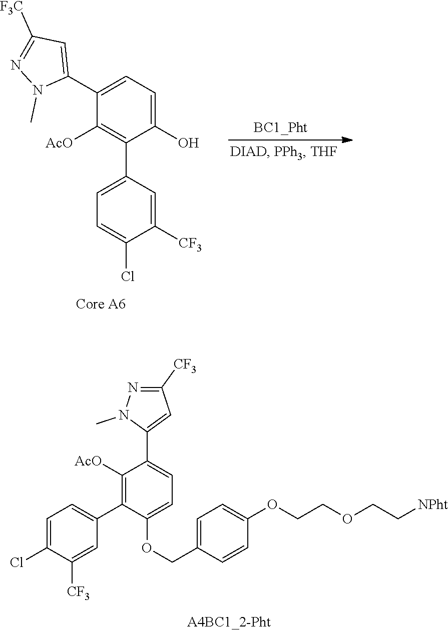

To a solution of Core A6 (60 mg, 125.32 μmol, 1 eq), BC1_Pht (64.17 mg, 187.98 μmol, 1.5 eq) in THF (2 mL) was added PPh3 (164.35 mg, 626.60 μmol, 5 eq), DIAD (109.13 mg, 626.60 μmol, 113.91 μL, 5 eq) in one portion at 0° C. under N₂. The mixture was stirred at 25° C. for 12 h. TLC showed the reaction was completed. The mixture was added ice-water (5 mL). The aqueous phase was extracted with ethyl acetate (5 mL). The combined organic phase was washed with brine (5 mL), dried with anhydrous Na₂SO₄, filtered and concentrated in vacuum to give A4BC1_2Pht (100.5 mg, 99.98% yield, crude purity) as yellow oil.

### General Procedure for Preparation of A4BC1_NH2-ET37412-128

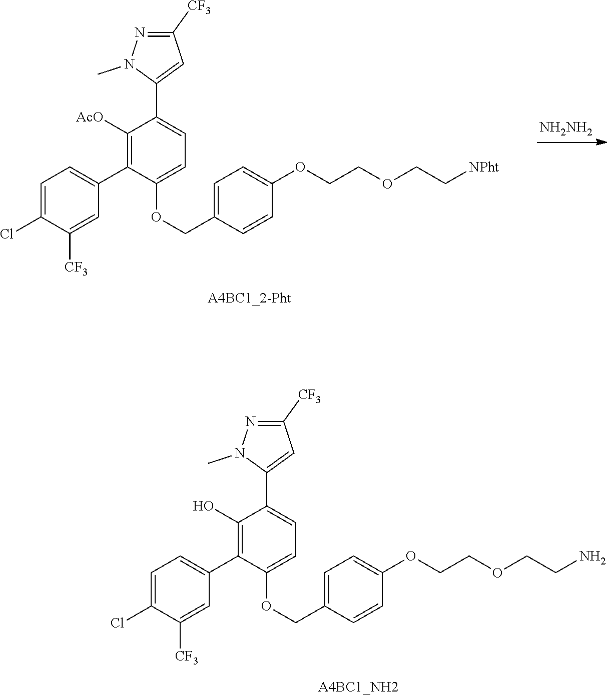

To a solution of A4BC1_2-Pht (400 mg, 498.68 μmol, 1 eq) in EtOH (5 mL) was added NH₂NH₂·H₂O (312.05 mg, 4.99 mmol, 302.96 μL, 80% purity, 10 eq) at 25° C. under N₂. The mixture was stirred at 25° C. for 1 hrs. LCMS showed the reaction was completed. The mixture was concentrated to give a residue. The residue was purified by Prep-HPLC (column: Waters Xbridge BEH C18 100*30 mm*10 um; mobile phase: [water (10 mM NH4HCO3)-ACN]; B %: 50%-75%, 8 min) to give A4BC1_NH2 (200 mg, 317.47 μmol, 63.66% yield) as white solid.

¹H NMR (ET37412-128-1, 400 MHz, CHLOROFORM-d) δ 2.96 (br s, 2H), 3.55-3.67 (m, 3H), 3.77-3.86 (m, 6H), 4.07-4.16 (m, 2H), 5.02 (s, 2H), 6.51-6.59 (m, 1H), 6.57 (s, 1H), 6.76 (br d, J=8.68 Hz, 1H), 6.88 (br d, J=8.44 Hz, 2H), 7.15 (br d, J=8.44 Hz, 2H), 7.20 (br d, J=8.56 Hz, 1H), 7.49-7.54 (m, 1H), 7.56-7.61 (m, 1H), 7.78 (s, 1H)

### General Procedure for Preparation of A4BC1R1 (NUCC-0226301)-ET37412-130

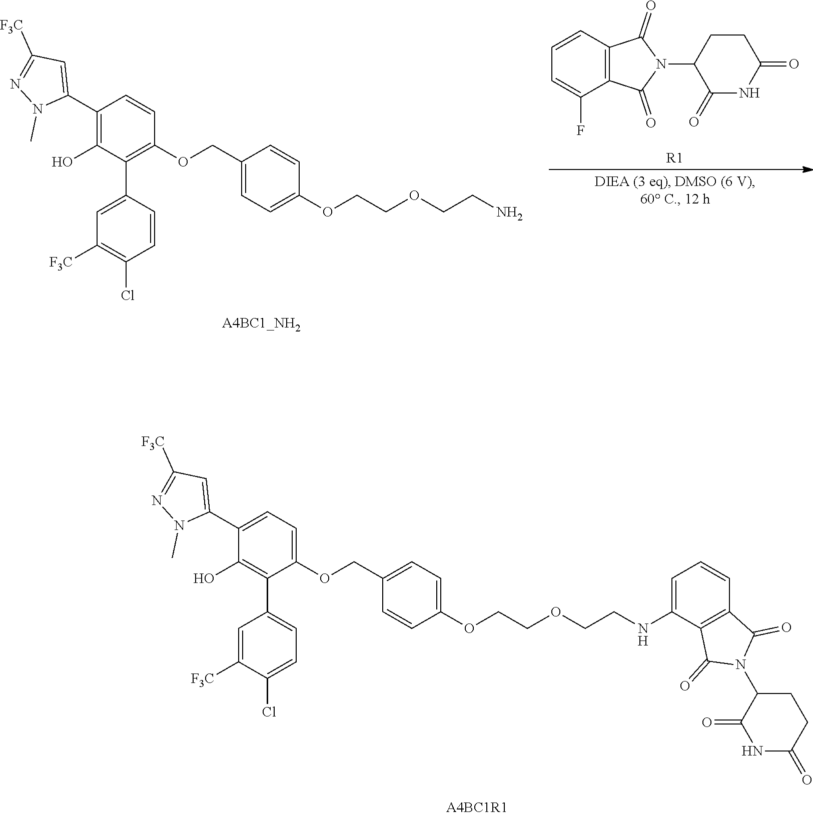

To a solution of A4BC1_NH2 (100 mg, 148.81 μmol, 1 eq) in DMSO (2 mL) was added DIEA (57.70 mg, 446.42 μmol, 77.76 μL, 3 eq) and R1 (61.65 mg, 223.21 μmol, 1.5 eq) at 25° C. under N₂. The mixture was stirred at 60° C. for 12 h. LCMS showed the reaction was completed. The mixture was filtered to give a residue which was purified by Prep-HPLC (column: Phenomenex Gemini-NX C18 75*30 mm*3 um; mobile phase: [water (0.05% NH₃H₂O+10 mM NH4HCO3)-ACN]; B %: 50%-85%, 8 min) to give A4BC1R1 (5 mg, 4% yield) as yellow solid.

¹H NMR (ET37412-130-11, 400 MHz, CHLOROFORM-d) δ 2.08-2.15 (m, 1H), 2.66-2.98 (m, 3H), 3.51 (t, J=5.32 Hz, 2H), 3.80 (t, J=5.38 Hz, 2H), 3.84 (s, 3H), 3.86-3.89 (m, 2H), 4.11-4.16 (m, 2H), 4.91 (dd, J=12.04, 5.32 Hz, 1H), 5.02 (s, 2H), 5.16-5.27 (m, 1H), 5.16-5.27 (m, 1H), 6.57 (s, 1H), 6.78 (d, J=8.56 Hz, 1H), 6.85-6.90 (m, 2H), 6.93 (d, J=8.56 Hz, 1H), 7.09 (d, J=6.97 Hz, 1H), 7.15 (d, J=8.68 Hz, 2H), 7.21 (d, J=8.56 Hz, 1H), 7.47 (dd, J=8.50, 7.15 Hz, 1H), 7.51-7.55 (m, 1H), 7.58-7.62 (m, 1H), 7.78 (d, J=1.83 Hz, 1H), 8.01 (s, 1H)

$$LCMS ( ESI + ) : RT = 3.602 min , m / z 886.2 {( M + 1 )}^{+} .$$

### 5_95CD_6min_MS1500-220-254-ELSD:

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H2O+10 mM NH4HCO3, mobile phase B was Acetonitrile. The column used for chromatography was a Xbridge C18 2.1*50 mm column (5 um particles). Detection methods are diode array (DAD) and evaporative light scattering (ELSD) detection. MS mode was positive electrospray ionization. MS range was 100-1500.

### General Procedure for Preparation of A4BC1R3 (NUCC-0226276)-ET37412-113

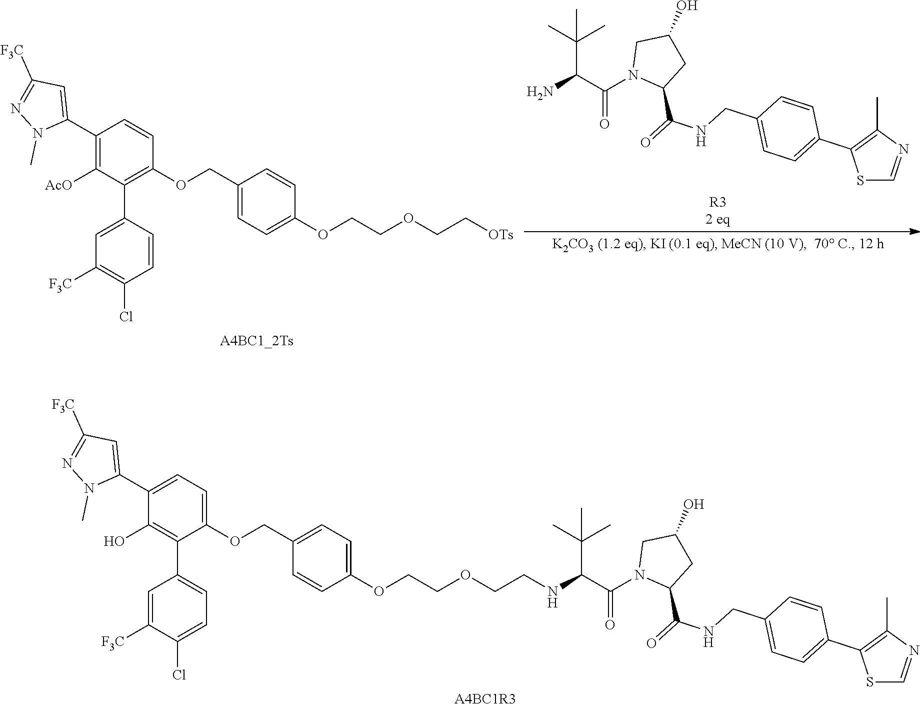 [SMILES: CC(=O)Oc1c(-c2cc(C(F)(F)F)nn2C)ccc(OCc2ccc(OCCOCCOS(=O)(=O)c3ccc(C)cc3)cc2)c1-c1ccc(Cl)c(C(F)(F)F)c1 ; Cc1ncsc1-c1ccc(CNC(=O)[C@@H]2C[C@@H](O)CN2C(=O)[C@@H](N)C(C)(C)C)cc1 ; Cc1ncsc1-c1ccc(CNC(=O)[C@@H]2C[C@@H](O)CN2C(=O)[C@@H](NCCOCCOc2ccc(COc3ccc(-c4cc(C(F)(F)F)nn4C)c(O)c3-c3ccc(Cl)c(C(F)(F)F)c3)cc2)C(C)(C)C)cc1]

To a solution of A4BC1_2 Ts (100 mg, 120.89 μmol, 1 eq) and R³ (104.10 mg, 241.78 μmol, 2 eq) in MeCN (1 mL) was added K2CO3 (33.42 mg, 241.78 μmol, 2 eq) and KI (2.01 mg, 12.09 μmol, 0.1 eq). The mixture was stirred at 70° C. for 12 h. LCMS showed the reaction was completed. The mixture was filtered and concentrated to give a residue which was purified by Prep-HPLC (column: Waters Xbridge BEH C18 100*30 mm*10 um; mobile phase: [water (10 mM NH4HCO3)-ACN]; B %: 50%-80%, 10 min) to give A4BC1R3 (5 mg, 4% yield) as white solid.

¹H NMR (ET37412-113-1, 400 MHz, METHANOL-d₄) δ 0.99 (s, 9H), 1.95-2.27 (m, 2H), 2.45 (s, 3H), 2.55-2.82 (m, 2H), 3.39 (br s, 2H), 3.58-3.69 (m, 2H), 3.72-3.91 (m, 5H), 4.12 (br d, J=4.16 Hz, 1H), 4.29-4.46 (m, 2H), 4.50-4.66 (m, 4H), 5.01 (br s, 1H), 6.58 (s, 1H), 6.80-6.94 (m, 3H), 7.13-7.19 (m, 2H), 7.23 (d, J=8.56 Hz, 1H), 7.34-7.49 (m, 3H), 7.59 (q, J=8.15 Hz, 2H), 7.74 (s, 1H), 8.85 (s, 1H)

$$LCMS ( ESI + ) : RT = 2.662 min , m / z 1043.3 {( M + 1 )}^{+} .$$

### 5_95AB_6min_MS1500-220-254-ELSD:

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Luna C18 50*2.0 mm column (5 um particles). Detection methods are diode array (DAD) and evaporative light scattering (ELSD) detection. MS mode was positive electrospray ionization. MS range was 100-1500.

### General Procedure for Preparation of A4BC1R4 (NUCC-0226302)-ET37412-131

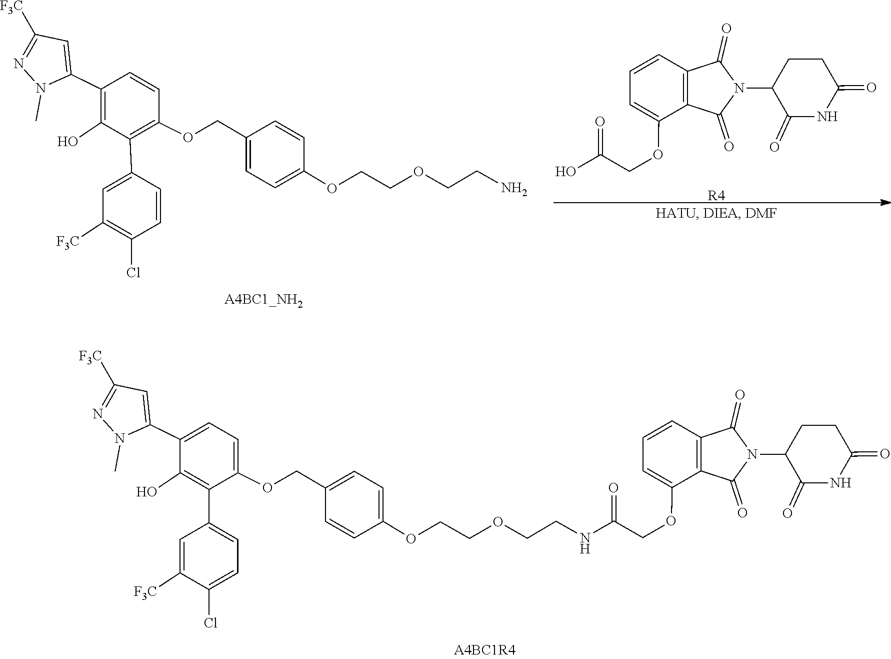

To a solution of A4BC1_NH2 (100 mg, 158.74 μmol, 1 eq) in DMF (2 mL) was added R⁴ (79.11 mg, 238.10 μmol, 1.5 eq), DIEA (61.55 mg, 476.21 μmol, 82.94 μL, 3 eq) and HATU (90.53 mg, 238.10 μmol, 1.5 eq) at 25° C. under N₂. The reaction was stirred at 25° C. for 12 h. LCMS showed the reaction was completed. The reaction was filtered to give a residue which was purified by Prep-HPLC (column: Phenomenex Gemini-NX C18 75*30 mm*3 um; mobile phase: [water (0.05% NH3H2O+10 mM NH4HCO3)-ACN]; B %: 40%-75%, 8 min) to give A4BC1R4 (9 mg, 6% yield) as white solid.

¹H NMR (ET37412-131-yl, 400 MHz, CHLOROFORM-d) δ 1.94-1.99 (m, 1H), 2.40-2.55 (m, 1H), 2.60-2.72 (m, 2H), 3.49-3.58 (m, 2H), 3.61-3.69 (m, 2H), 3.76-3.80 (m, 5H), 4.02-4.12 (m, 2H), 4.54 (s, 2H), 4.77 (dd, J=12.59, 5.38 Hz, 1H), 4.91 (s, 2H), 5.23 (br s, 1H), 6.49 (s, 1H), 6.69 (d, J=8.68 Hz, 1H), 6.75-6.81 (m, 2H), 7.05 (dd, J=12.41, 8.62 Hz, 3H), 7.14 (d, J=8.56 Hz, 1H), 7.42-7.48 (m, 2H), 7.50-7.54 (m, 1H), 7.57 (br t, J=5.26 Hz, 1H), 7.63 (dd, J=8.31, 7.46 Hz, 1H), 7.70 (d, J=1.83 Hz, 1H), 7.88 (s, 1H)

$$LCMS ( ESI + ) \text{:} RT = 3.3 \; 46 min , m / z 944.1 {( M + 1 )}^{+} .$$

### 5_95CD_6min_MS1500-220-254-ELSD:

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H2O+10 mM NH4HCO3, mobile phase B was Acetonitrile. The column used for chromatography was a Xbridge C18 2.1*50 mm column (5 um particles). Detection methods are diode array (DAD) and evaporative light scattering (ELSD) detection. MS mode was positive electrospray ionization. MS range was 100-1500.

### General Procedure for Preparation of A4BC1R5 (NUCC-0226277)-ET37412-112

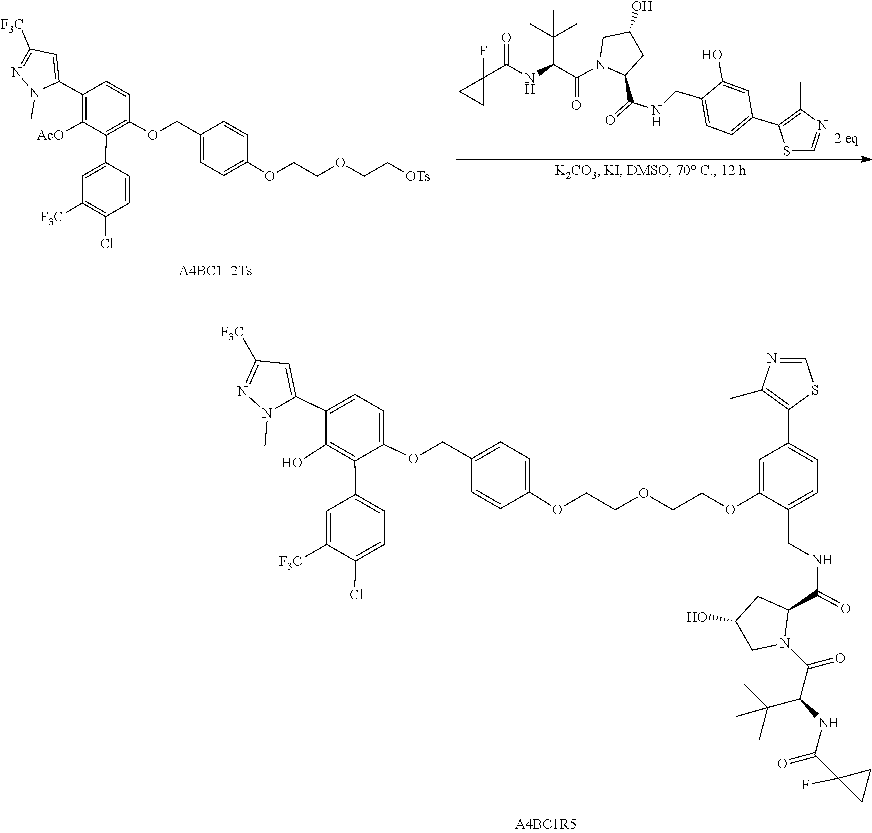 [SMILES: CC(=O)Oc1c(-c2cc(C(F)(F)F)nn2C)ccc(OCc2ccc(OCCOCCOS(=O)(=O)c3ccc(C)cc3)cc2)c1-c1ccc(Cl)c(C(F)(F)F)c1 ; Cc1ncsc1-c1ccc(CNC(=O)[C@@H]2C[C@@H](O)CN2C(=O)[C@@H](NC(=O)C2(F)CC2)C(C)(C)C)c(O)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOc2ccc(COc3ccc(-c4cc(C(F)(F)F)nn4C)c(O)c3-c3ccc(Cl)c(C(F)(F)F)c3)cc2)c1]

To a solution of A4BC1_2 Ts (100 mg, 120.89 μmol, 1 eq) and R⁵ (128.78 mg, 241.78 μmol, 2 eq) in DMSO (1 mL) was added K₂CO₃ (33.42 mg, 241.78 μmol, 2 eq) and KI (2.01 mg, 12.09 μmol, 0.1 eq). The mixture was stirred at 70° C. for 12 h. LCMS showed the reaction was completed. The mixture was added ice-water (1 mL). The aqueous phase was extracted with ethyl acetate (2 mL). The organic phase was concentrated in vacuum to give residue which was purified by Prep-HPLC (column: Waters Xbridge BEH C18 100*30 mm*10 um; mobile phase: [water (10 mM NH4HCO3)-ACN]; B %: 55%-85%, 10 min) to give A4BC1R5 (8 mg, 6% yield) as white solid.

¹H NMR (ET37412-112-1, 400 MHz, METHANOL-d₄) δ 1.01 (s, 9H), 1.20-1.41 (m, 4H), 1.98-2.24 (m, 2H), 2.46 (s, 3H), 3.72-3.86 (m, 5H), 3.95 (br d, J=12.35 Hz, 4H), 4.14 (br s, 2H), 4.25 (br s, 2H), 4.34-4.44 (m, 1H), 4.46 (br s, 1H), 4.56-4.66 (m, 2H), 4.73 (s, 1H), 5.01 (s, 2H), 6.58 (s, 1H), 6.85 (br d, J=8.16 Hz, 3H), 6.96-7.09 (m, 2H), 7.15 (br d, J=7.94 Hz, 2H), 7.23 (br d, J=8.60 Hz, 1H), 7.45 (d, J=7.72 Hz, 1H), 7.53-7.64 (m, 2H), 7.74 (s, 1H), 8.80 (s, 1H)

$$LCMS ( ESI + ) : RT = 3.067 min , m / z 1145.3 {( M + 1 )}^{+} .$$

### 5_95AB_6min_MS1500-220-254-ELSD:

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Luna C18 50*2.0 mm column (Sum particles). Detection methods are diode array (DAD) and evaporative light scattering (ELSD) detection. MS mode was positive electrospray ionization. MS range was 100-1500.

### Synthesis of NUCC-0226258, NUCC-0226259, NUCC-0226260, NUCC-0226261

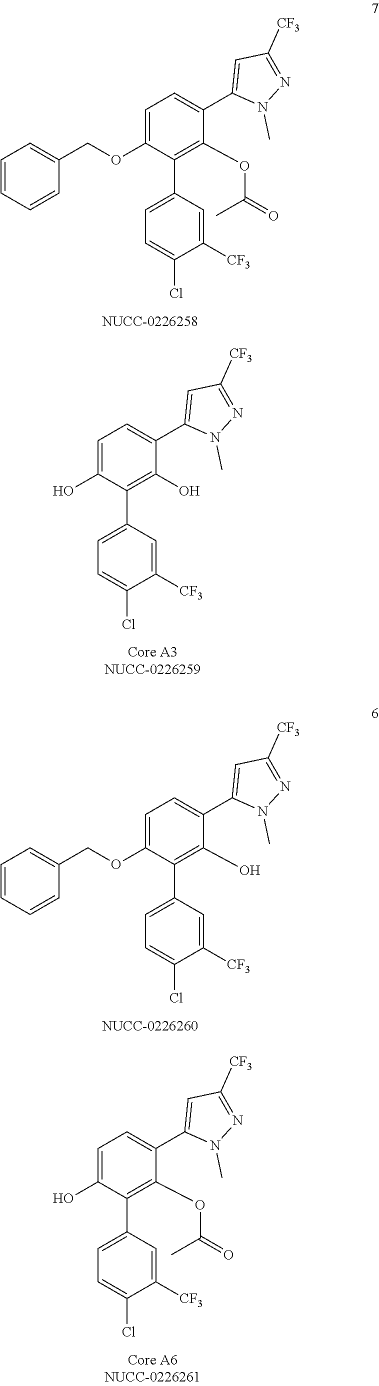 [SMILES: CC(=O)Oc1c(-c2cc(C(F)(F)F)nn2C)ccc(OCc2ccccc2)c1-c1ccc(Cl)c(C(F)(F)F)c1 ; Cn1nc(C(F)(F)F)cc1-c1ccc(O)c(-c2ccc(Cl)c(C(F)(F)F)c2)c1O ; Cn1nc(C(F)(F)F)cc1-c1ccc(OCc2ccccc2)c(-c2ccc(Cl)c(C(F)(F)F)c2)c1O ; CC(=O)Oc1c(-c2cc(C(F)(F)F)nn2C)ccc(O)c1-c1ccc(Cl)c(C(F)(F)F)c1]

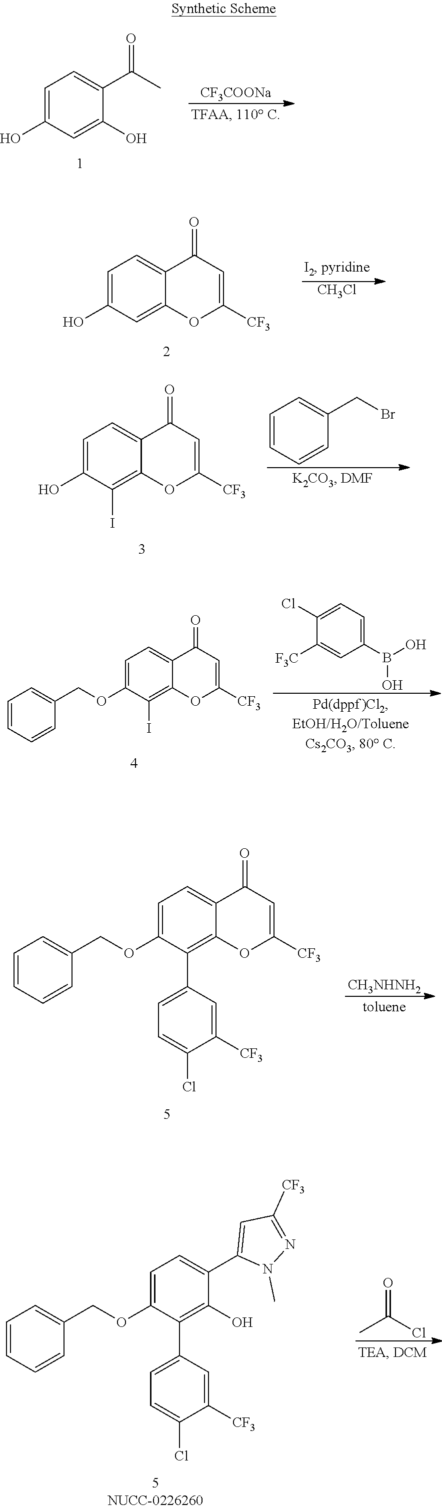

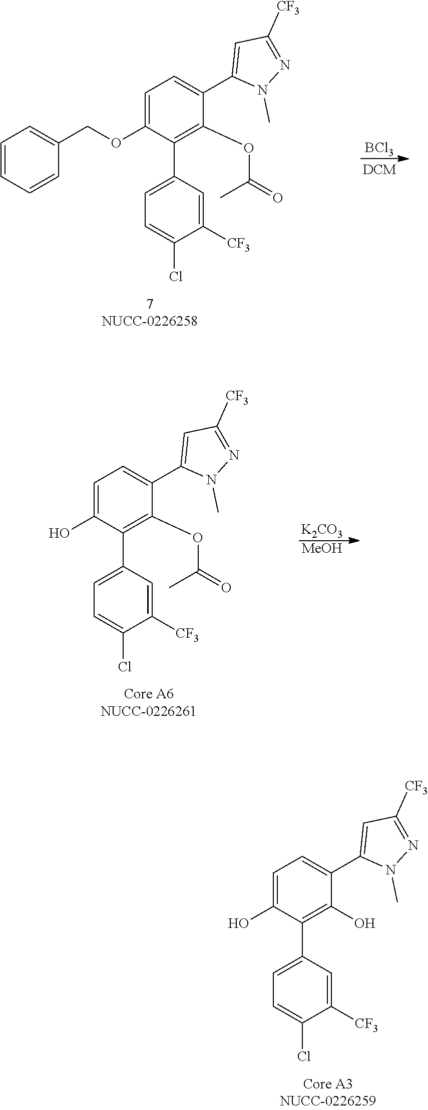

### Experimental for Largest Scale Run

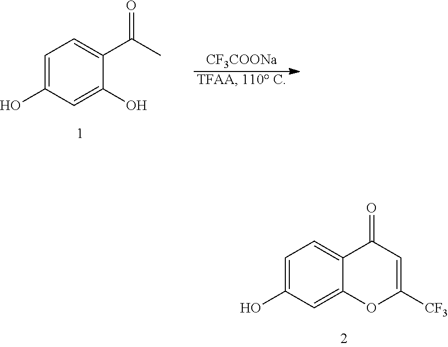

To a suspension of Compound 1 (10 g, 65.73 mmol, 8.47 mL, 1 eq) in TFAA (37 ml) was placed in a high pressure tube (100 mL). Sodium trifluoroacetate (19.67 g, 144.60 mmol, 2.2 eq) was added and the system was capped and stirred at 130° C. for 24 h. Totally 30 batches were set as parallel reaction. LCMS showed all starting materials consumed. The reaction was allowed to cool to 25° C. and combined and then was diluted with EtOAc (1 L). The mixture was neutralized by saturated aqueous K₂CO₃ solution until no more bubbling was observed. The organic layer was separated and the aqueous portion was extracted with EtOAc (3×500 mL). The organic layer was washed by brine, dried over anhydrous Na₂SO₄, concentrated to ⅓ volume of EtOAc and the flask was allowed to stand at 25° C. for 8 hrs. Compound 2 (180 g, 793.0 mmol, 40.0% yield) was collected as white solid.

### Reaction LCMS:

### LCMS Method:

$$LCMS ( ESI + ) : m / z = 231.1 ( M + H ) + , RT : 0.806 min .$$

5_95AB_2_min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

### General Procedure for Preparation of Compound 3-ET32240-1059

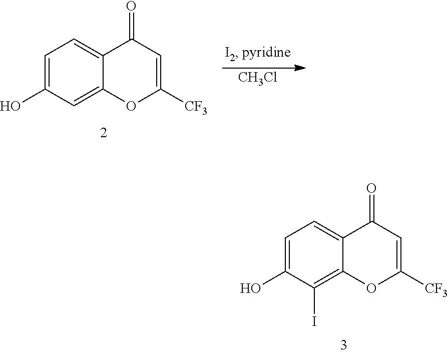 [SMILES: O=c1cc(C(F)(F)F)oc2cc(O)ccc12 ; "CH3Cl" ; O=c1cc(C(F)(F)F)oc2c(I)c(O)ccc12]

A solution of Compound 2 (180 g, 782.13 mmol, 1 eq), iodine (794.05 g, 3.13 mol, 630.20 mL, 4 eq), pyridine (247.47 g, 3.13 mol, 252.52 ml, 4 eq) in chloroform (I L) was stirred at 25° C. for 8 h. LCMS showed the reaction completed. The mixture was poured into water (500 mL) and triturated with petroleum ether:ethyl acetate (10:1, 800 mL) to give Compound 3 (210 g, 589.83 mmol, 75.41% yield) as yellowish solid.

### Reaction LCMS:

### LCMS Method:

$$LCMS ( ESI + ) : m / z = 356.9 ( M + H ) + , RT : 0.89 min .$$

5_95AB_2_min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (Sum particles). Detection methods are diode array (DAD) and evaporative light scattering (ELSD) detection as well as positive electrospray ionization. MS range was 100-1000.

¹H NMR (400 MHz, METHANOL-d₄) δ ppm 6.79 (s, 1H) 7.02 (d, J=8.82 Hz, 1H) 7.95-7.99 (m, 1H)

### General Procedure for Preparation of Compound 4-ET32240-1078

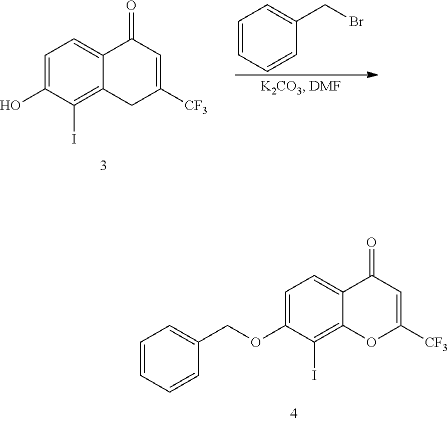 [SMILES: O=C1C=C(C(F)(F)F)Cc2c1ccc(O)c2I ; BrCc1ccccc1 ; O=c1cc(C(F)(F)F)oc2c(I)c(OCc3ccccc3)ccc12]

A solution of Cpd 3 (130 g, 365.13 mmol, 1 eq), bromomethylbenzene (74.94 g, 438.16 mmol, 52.04 mL, 1.2 eq), bromomethylbenzene (74.94 g, 438.16 mmol, 52.04 mL, 1.2 eq) in acetone (1 L) was added potassium carbonate (100.93 g, 730.26 mmol, 2 eq). The mixture was stirred at 80° C. for 8 h. TLC showed the reaction completed. The mixture was poured into water (500 mL) and extracted with ethyl acetate (3×500 mL). The organic layer was dried over Na₂SO₄ and concentrated to give crude product, which was purified by chromatography on silica, eluted with petroleum ether:ethyl acetate=10:1 to 5:1 to give desired product Cpd 4 (120 g, 268.96 mmol, 73.66% yield) as yellowish solid.

¹H NMR (400 MHz, CHLOROFORM-d) δ ppm 5.27 (s, 2H) 6.63 (s, 1H) 6.96 (d, J=8.93 Hz, 1H) 7.10-7.39 (m, 8H) 7.40-7.46 (m, 2H) 8.08 (d, J=8.93 Hz, 1H)

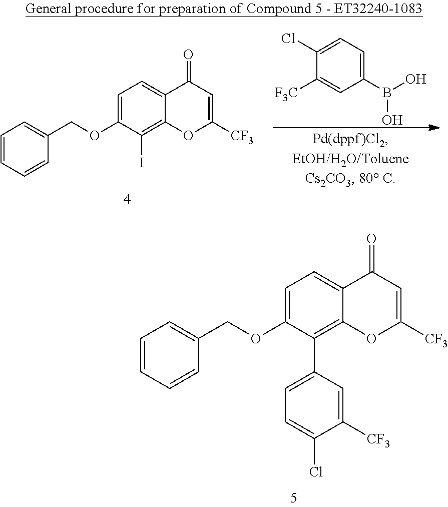 [SMILES: O=c1cc(C(F)(F)F)oc2c(I)c(OCc3ccccc3)ccc12 ; OB(O)c1ccc(Cl)c(C(F)(F)F)c1 ; O=c1cc(C(F)(F)F)oc2c(-c3ccc(Cl)c(C(F)(F)F)c3)c(OCc3ccccc3)ccc12]

A solution of Cpd 4 (100 g, 22.41 mmol, 1 eq), [4-chloro-3-(trifluoromethyl)phenyl]boronic acid (5.03 g, 22.41 mmol, 1 eq), cesium carbonate (14.61 g, 44.83 mmol, 2 eq) in toluene (2 L) and ethanol (400 mL) and water (80 mL) was added Pd(dppf)Cl₂ (1.64 g, 2.24 mmol, 0.1 eq) under N₂ atmosphere. The mixture was stirred at 80° C. for 8 h. TLC showed the reaction completed. The mixture was poured into water (500 mL) and extracted with ethyl acetate (3×500 mL). The organic layer was dried over Na₂SO₄ and concentrated to give crude product which was triturated with ethyl acetate:petroleum ether (1:10, 500 mL) to give desired product Cpd 5 (93 g, 18.64 mmol, 83.18% yield) as yellowish solid.

¹H NMR (400 MHz, CHLOROFORM-d) δ ppm 5.26 (s, 2H) 6.71 (s, 1H) 7.27 (d, J=2.93 Hz, 2H) 7.34-7.41 (m, 3H) 7.55-7.66 (m, 2H) 7.85 (s, 1H) 8.26 (d, J=8.93 Hz, 1H)

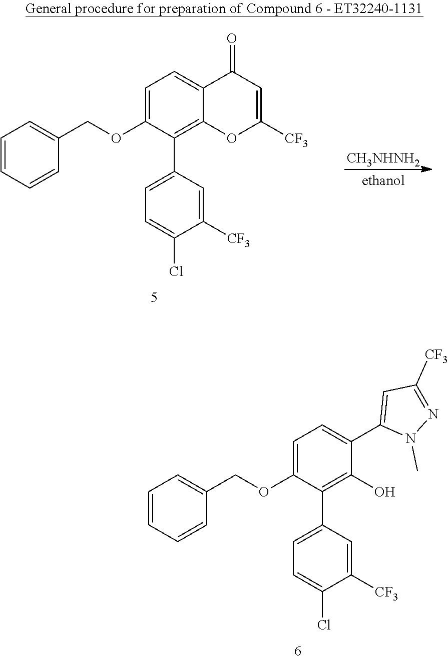

A suspension of Cpd 5 (100 g, 200.48 mmol, 1 eq), methylhydrazine (27.71 g, 601.44 mmol, 31.67 mL, 3 eq) in ethanol (500 mL) was bubbled with N₂ gas for 10 minutes. The vial was then heated at 80° C. for 16 h. LCMS showed all starting materials consumed. The resulting mixture was concentrated to give crude residue which was triturated by toluene to obtain Cpd 6 (75 g, 142.35 mmol, 71.01% yield) as brown solid.

$$LCMS ( ESI + ) : m / z = 527.2 ( M + H ) + , RT : 1.203 min .$$

5_95AB_2_min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

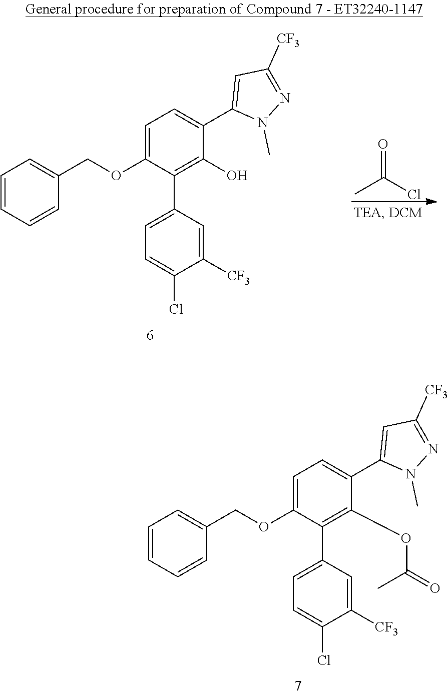

A solution of Cpd 6 (42 g, 79.72 mmol, 1 eq) in DCM (500 mL) was added acetyl chloride (7.51 g, 95.66 mmol, 6.83 mL, 1.2 eq) and TEA (9.68 g, 95.66 mmol, 13.31 ml, 1.2 eq). The mixture was stirred at 25° C. for 8 hrs. TLC showed all starting materials consumed. Once completion, the mixture was poured into water and extracted with DCM (2×100 mL). The organic layer was dried over Na₂SO₄, concentrated to give Cpd 7 (45 g, 79.10 mmol, 99.23% yield) as white solid which was used directly in next step.

¹H NMR (400 MHz, CHLOROFORM-d) δ 1.72-1.81 (m, 3H), 3.75-3.84 (m, 3H), 5.10-5.16 (m, 2H), 6.45-6.52 (m, 1H), 7.07 (d, J=8.60 Hz, 1H), 7.21-7.27 (m, 3H), 7.38 (s, 2H), 7.44-7.49 (m, 1H), 7.51-7.57 (m, 1H), 7.72-7.76 (m, 1H)

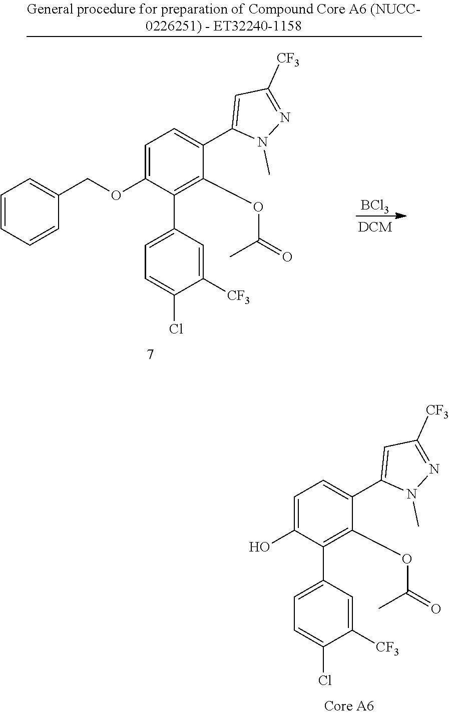 [SMILES: CC(=O)Oc1c(-c2cc(C(F)(F)F)nn2C)ccc(OCc2ccccc2)c1-c1ccc(Cl)c(C(F)(F)F)c1 ; "BCl3" ; CC(=O)Oc1c(-c2cc(C(F)(F)F)nn2C)ccc(O)c1-c1ccc(Cl)c(C(F)(F)F)c1]

A solution of Cpd 7 (50 g, 87.89 mmol, 1 eq) in DCM (200 mL) was cooled to 0° C. BC13 (1 M, 175.78 mL, 2 eq) was added to the mixture dropwise and maintain the temperature below 0° C. The mixture was stirred at 0° C. for 4 hrs. TLC showed all starting materials consumed. On completion, the mixture was added ice (100 mL) and separate the organic layer. The aqueous was extracted with EtOAc (3×100 mL). The organic layer was dried over Mg₂SO₄ and concentrated to give product which was purified by chromatography on silica, eluted with PE:EA=10:1 to 1:1 give Cpd A6 (25 g, 52.22 mmol, 59.41% yield) as white solid.

¹H NMR (400 MHz, CHLOROFORM-d) δ 1.78 (s, 3H), 3.76-3.84 (m, 3H), 5.95 (s, 1H), 6.48-6.53 (m, 1H), 6.96-7.02 (m, 1H), 7.23-7.28 (m, 1H), 7.48-7.53 (m, 1H), 7.62 (d, J=8.16 Hz, 1H), 7.70-7.74 (m, 1H)

### Synthesis of Target D (NUCC-0226219) and Target E (NUCC-0226223)

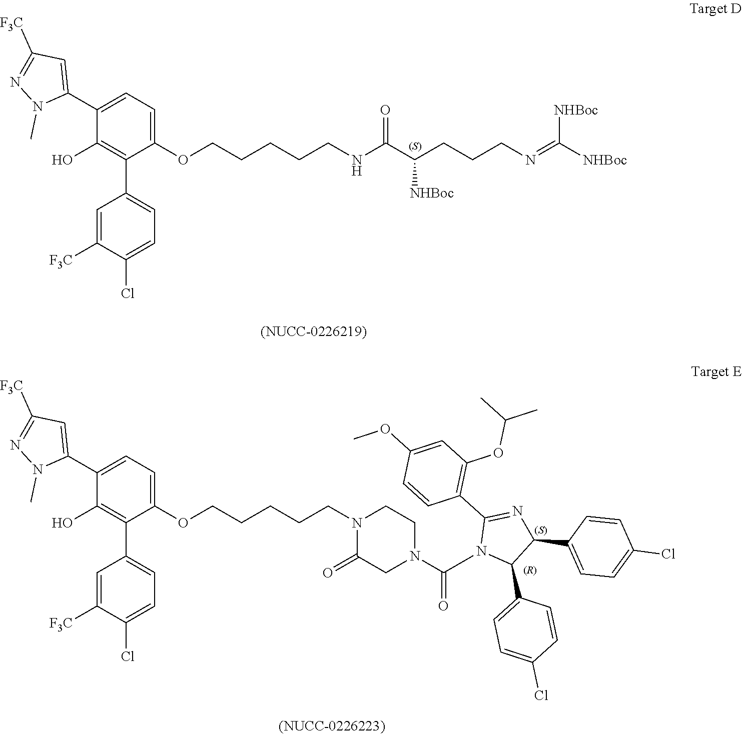

### Synthetic Scheme

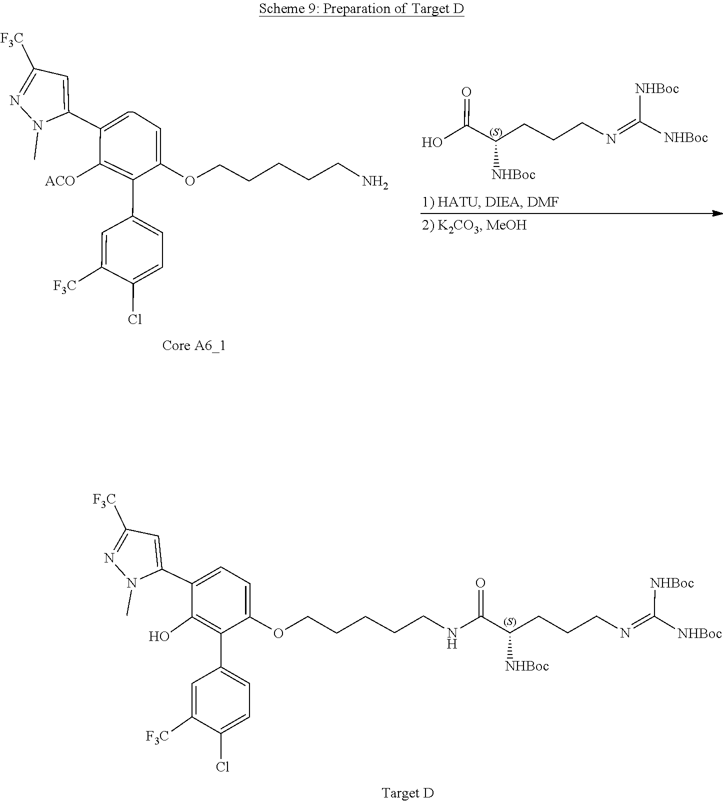

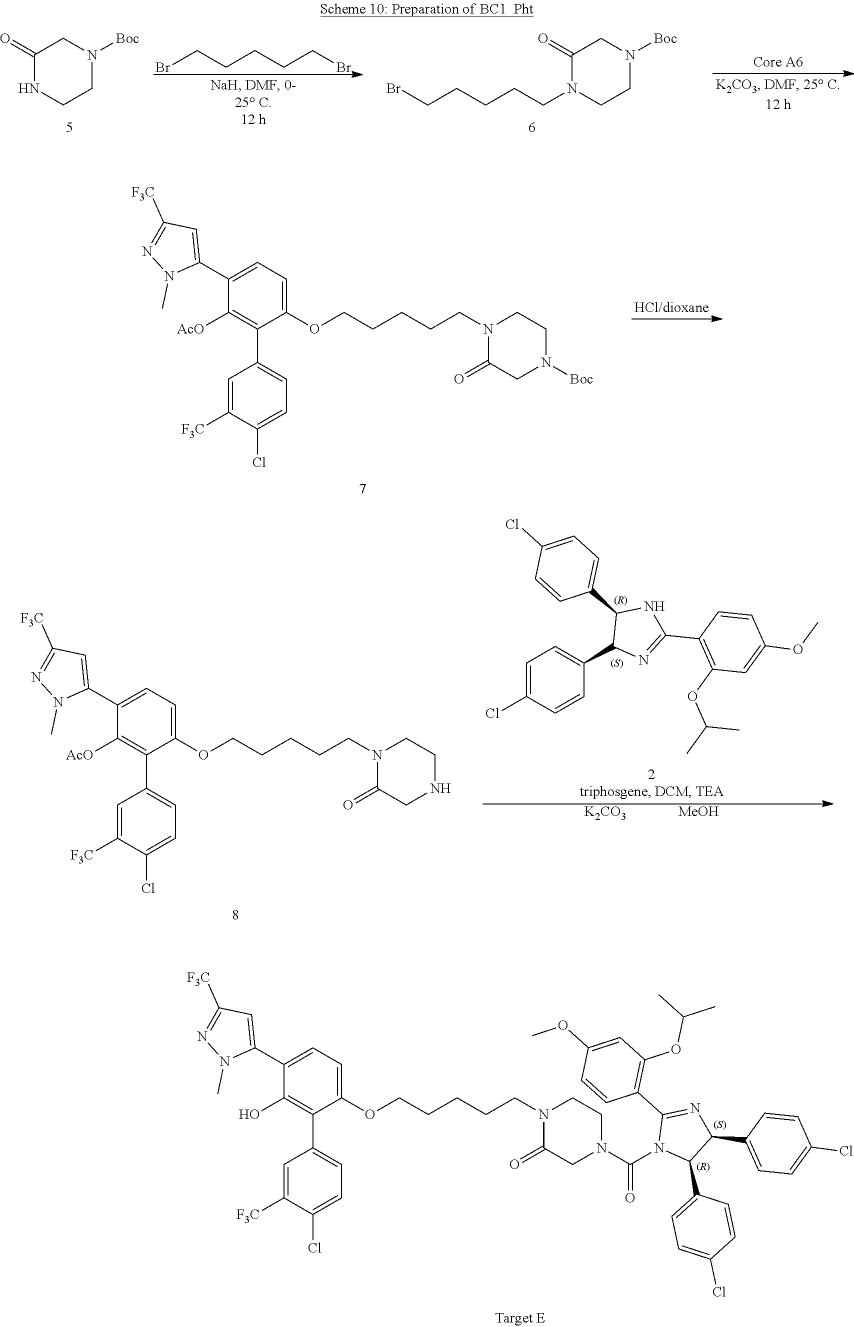

### Experimentals for Largest Scale Run

### General Procedure for Preparation of Target DY (NHCC-0226219)-1737412-4

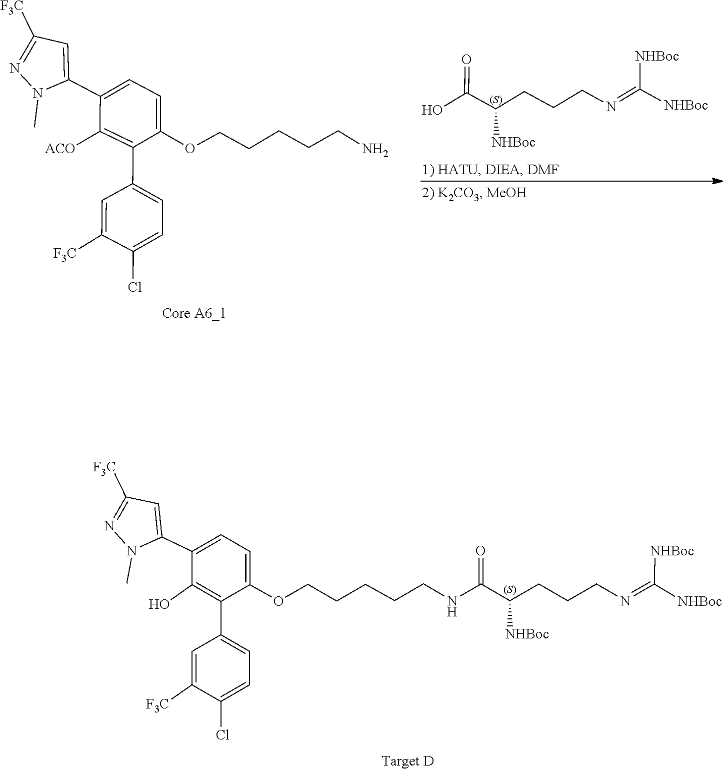

To a mixture of Core A6_1 (55 mg, 97.53 μmol, 1 eq) and (2S)-5-[bis(tert-butoxycarbonylamino)methyleneamino]-2-(tert-butoxycarbonylamino) pentanoic acid (55.54 mg, 117.04 μmol, 1.2 eq) in DMF (1 mL) was added HATU (44.50 mg, 117.04 μmol, 1.2 eq) and DIEA (18.91 mg, 146.30 μmol, 1.5 eq) in one portion. The reaction was stirred at 25° C. for 12 hr. And then to the reaction mixture was added K₂CO₃ (50 mg) and then reaction was stirred at 25° C. for 2 h. LCMS showed the reaction was completed. The reaction was filtered to give the residue which was purified by Prep-HPLC (column: Phenomenex Gemini-NX C18 75*30 mm*3 um; mobile phase: [water (10 mM NH4HCO3)-ACN]; B %: 65%-95%, 8 min) to give Target D (11 mg, 11% yield) as white solid.

¹H NMR (ET37412-4-1, 400 MHz, DMSO-d6) δ 0.78-0.87 (m, 2H), 1.02 (br s, 4H), 1.36 (d, J=12.28 Hz, 19H), 1.45 (s, 12H), 2.77-2.91 (m, 2H), 3.07 (br s, 2H), 3.24 (br s, 2H), 3.78 (s, 4H), 6.56-6.71 (m, 1H), 6.61-6.68 (m, 1H), 6.61-6.68 (m, 1H), 6.66 (s, 1H), 6.76 (br d, J=8.23 Hz, 1H), 7.07 (br s, 1H), 7.64 (br s, 1H), 7.68-7.79 (m, 2H), 7.90 (s, 1H), 8.26 (br t, J=5.60 Hz, 1H), 11.48 (br s, 1H)

$$LCMS ( ESI + ) : RT = 3.051 min , m / z 978.3 {( M + 1 )}^{+} .$$

### 5_95AB_6min-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

### General Procedure for Preparation of Cpd 6 (NUCC-0226260)-ET37412

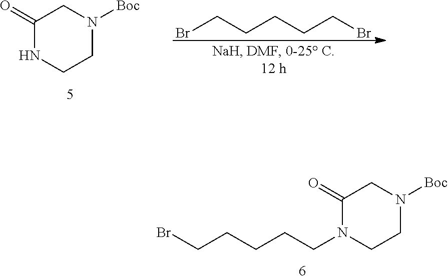 [SMILES: CC(C)(C)OC(=O)N1CCNC(=O)C1 ; BrCCCCCBr ; CC(C)(C)OC(=O)N1CCN(CCCCCBr)C(=O)C1]

To a mixture of Cpd 5 (5 g, 24.97 mmol, 1 eq) in DMF (60 mL) was added NaH (1.50 g, 37.46 mmol, 60% purity, 1.5 eq). After stirred for 1 h, 1,5-dibromopentane (6.89 g, 29.96 mmol, 4.05 mL, 1.2 eq) was added to the mixture. The reaction was stirred for 12 h at 25° C. The residue was poured into ice-water (100 mL). The aqueous phase was extracted with ethyl acetate (100 ml). The combined organic phase was dried with anhydrous Na₂SO₄, filtered and concentrated in vacuum to give the residue which was purified by silica gel chromatography (column height: 250 mm, diameter: 100 mm, 100-200 mesh silica gel, Petroleum ether/Ethyl acetate=1/0, 0/1) to afford Cpd 6 (6 g, 69% yield) as colorless oil.

1H NMR (ET37412-34-1, 400 MHz, CHLOROFORM-d) δ 1.49 (s, 8H), 1.56-1.65 (m, 4H), 1.77-2.01 (m, 2H), 3.27-3.53 (m, 5H), 3.57-3.73 (m, 2H), 3.96-4.22 (m, 3H)

### General Procedure for Preparation of Cpd 7-ET37412-71

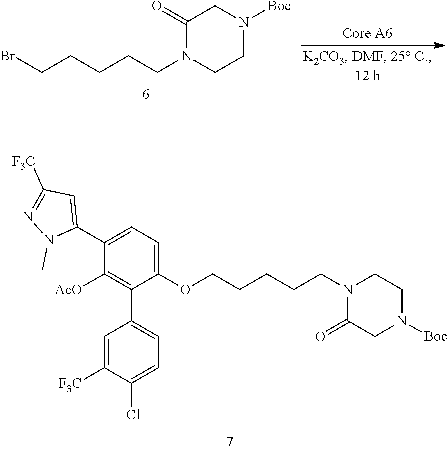 [SMILES: CC(C)(C)OC(=O)N1CCN(CCCCCBr)C(=O)C1 ; CC(=O)Oc1c(-c2cc(C(F)(F)F)nn2C)ccc(OCCCCCN2CCN(C(=O)OC(C)(C)C)CC2=O)c1-c1ccc(Cl)c(C(F)(F)F)c1]

To a mixture of core A6 (830 mg, 1.73 mmol, 1 eq) and Cpd 6 (1.14 g, 2.60 mmol, 80% purity, 1.5 eq) in DMF (5 mL) was added K₂CO₃ (359.40 mg, 2.60 mmol, 1.5 eq) in one portion at 25° C. The reaction was stirred at 25° C. for 12 h. LCMS showed the reaction was completed. The reaction was poured into water (10 mL) and extracted with ethyl acetate (20 mL). The organic layer was concentrated to give a residue which was purified by silica gel chromatography eluted with petroleum ether/ethyl acetate=1:0˜0:1 to give Cpd 7 (400 mg, 31% yield) as colorless oil.

$$LCMS ( ESI + ) \text{:} RT = 0.948 min , m / z 691.3 {( M + 1 )}^{+} .$$

### 5-95AB_2_min

LC/MS (The column used for chromatography was a Kinetex 5 μm EVO C18 100A 2.1*30 mm. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

### General Procedure for Preparation of Cpd 8-ET37412-48

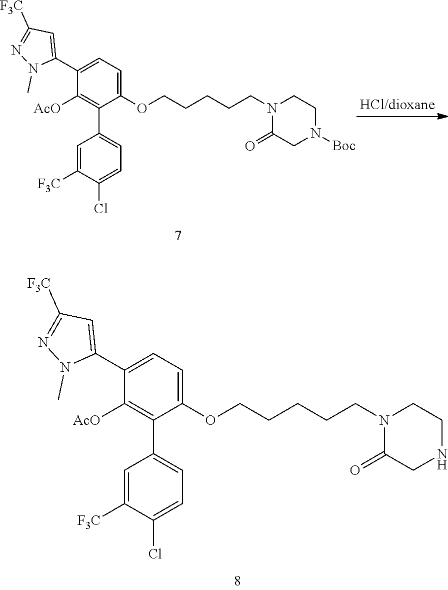 [SMILES: CC(=O)Oc1c(-c2cc(C(F)(F)F)nn2C)ccc(OCCCCCN2CCN(C(=O)OC(C)(C)C)CC2=O)c1-c1ccc(Cl)c(C(F)(F)F)c1 ; CC(=O)Oc1c(-c2cc(C(F)(F)F)nn2C)ccc(OCCCCCN2CCNCC2=O)c1-c1ccc(Cl)c(C(F)(F)F)c1]

To a mixture of Cpd 7 (400 mg, 1 eq) in dioxane (0.5 mL) was added HCl/dioxane (1 mL). The reaction mixture was stirred at 25° C. for 12 h. LCMS showed the reaction was completed. The reaction was concentrated to give a residue which was used to next step without any purification to give Cpd 8 (330 mg, 91% yield, HCl) as yellow oil.

$$LCMS ( ESI + ) : RT = 0.773 min , m / z 647.7 {( M + 1 )}^{+} .$$

### 5-95AB_2_min:

LC/MS (The column used for chromatography was a Kinetex 5 μm EVO C18 100A 2.1*30 mm. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

### General Procedure for Preparation of Target E (NUCC-0226223)-ET37412-52

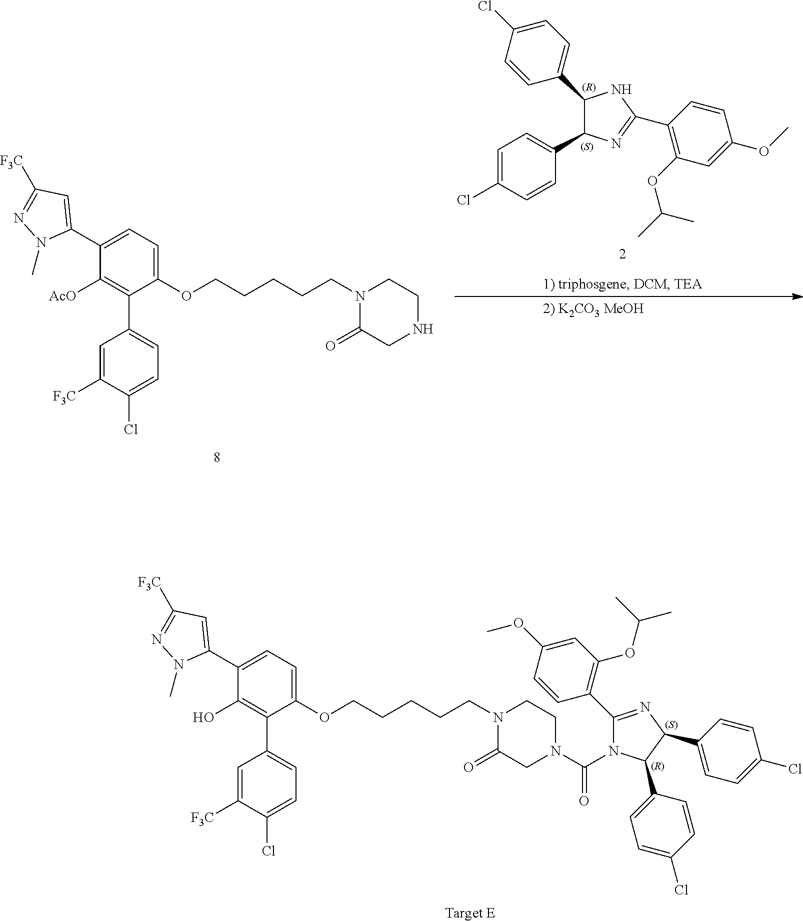

To a mixture of Cpd 8 (300.21 mg, 549.00 μmol, 1 eq) and Cpd 2 (250 mg, 549.00 μmol, 1 eq) in DCM (5 mL) was added triphosgene (407.29 mg, 1.37 mmol, 2.5 eq) and TEA (833.29 mg, 8.23 mmol, 1.15 mL, 15 eq) in one portion at 25° C. The reaction mixture was stirred at 25° C. for 12 h. After that, to the mixture was added K₂CO₃ (50 mg) and the reaction was stirred at 25° C. for 2 h. LCMS showed the reaction was completed. The reaction was poured into ice water (10 mL). The mixture was extracted with DCM (20 mL). The organic layer was concentrated to give a residue which was purified by Prep-HPLC (column: Phenomenex Gemini-NX 150*30 mm*5 um; mobile phase: [water (0.1% TFA)-ACN]; B %: 50%-80%, 9 min) to give Target E (0.045 g, 7% yield) as white solid.

¹H NMR (ET37412-52-P1B, 400 MHz, METHANOL-d₄) δ 1.17-1.28 (m, 2H), 1.31-1.39 (m, 2H), 1.45 (d, J=5.95 Hz, 6H), 1.61-1.71 (m, 2H), 2.97 (br s, 2H), 3.02-3.12 (m, 1H), 3.18-3.27 (m, 1H), 3.41-3.53 (m, 1H), 3.56-3.68 (m, 1H), 3.79 (s, 3H), 3.88 (s, 2H), 3.91 (s, 3H), 3.96-4.02 (m, 2H), 4.91 (dd, J=12.24, 6.06 Hz, 1H), 5.98 (d, J=11.03 Hz, 1H), 6.24 (d, J=11.03 Hz, 1H), 6.58 (s, 1H), 6.74-6.84 (m, 3H), 6.99 (d, J=8.38 Hz, 2H), 7.17 (dd, J=16.21, 8.49 Hz, 4H), 7.26 (dd, J=17.42, 8.60 Hz, 3H), 7.56-7.61 (m, 1H), 7.63-7.70 (m, 2H), 7.75 (d, J=1.76 Hz, 1H)

$$LCMS ( ESI + ) : RT = 2.966 min , m / z 1085. {( M + 1 )}^{+} .$$

### 5_95AB_6min_MS1500-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Luna C18 50*2.0 mm column (5 um particles). Detection methods are diode array (DAD) and evaporative light scattering (ELSD) detection. MS mode was positive electrospray ionization. MS range was 100-1500.

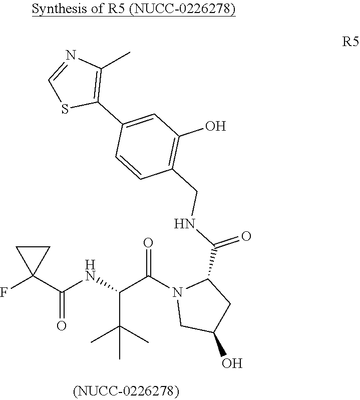 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1]

### Synthetic Scheme

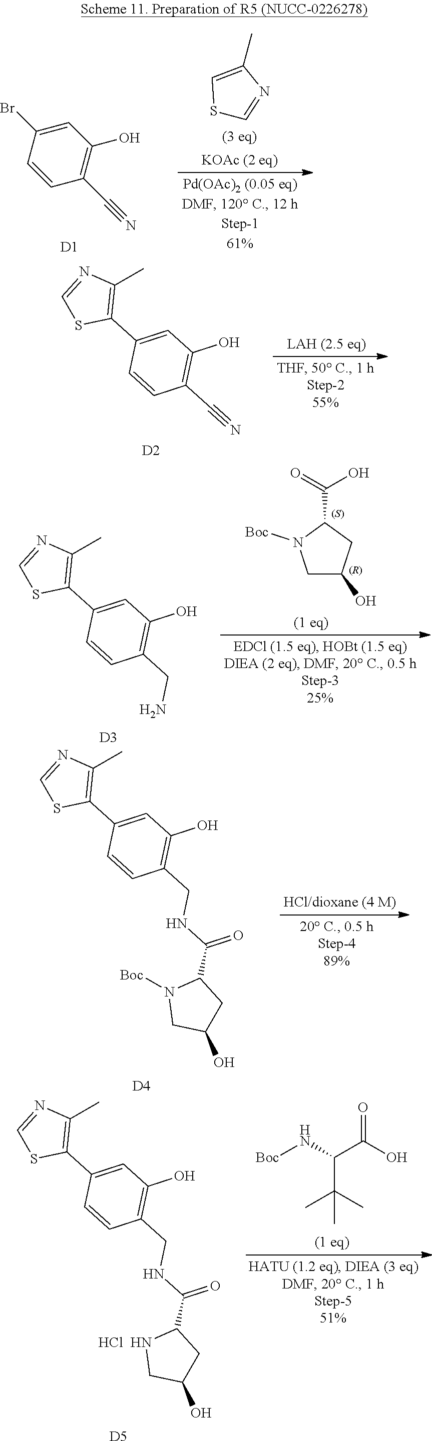

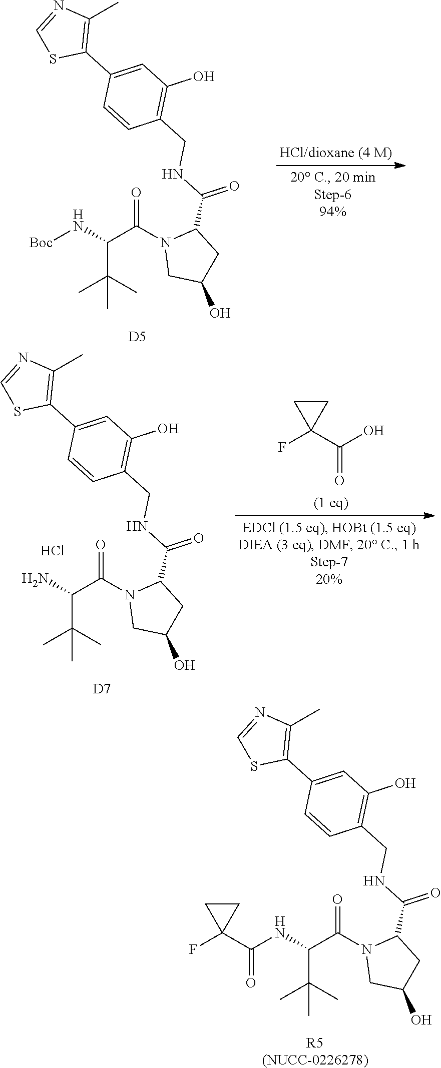

### Experimentals for Largest Scale Run

### General Procedure for Preparation of 2-hydroxy-4-(4-methylthiazol-5-yl)benzonitrile (D2)-ET39710-105

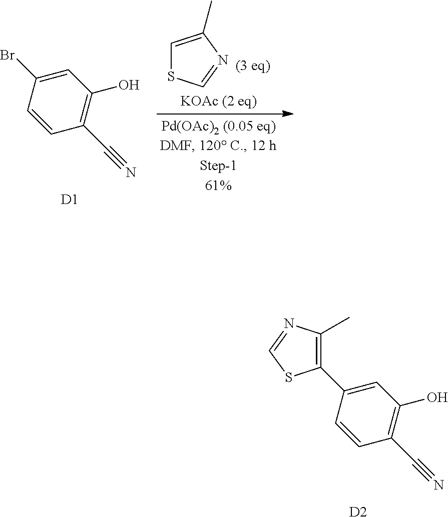

To a mixture of 4-bromo-2-hydroxy-benzonitrile (D1) (120 g, 606.01 mmol) in DMF (1 L) were added 4-methylthiazole (180.26 g, 1.82 mol), KOAc (118.95 g, 1.21 mol) and Pd(OAc)₂ (6.80 g, 30.30 mmol) at 20° C. under N₂. The reaction mixture was stirred at 120° C. for 12 h. LCMS showed the reaction was completed. The mixture was cooled to 20° C. and filtered over celite. The filtrate was poured into water (1 L) and extracted with EA (3×1.5 L). The combined organic layer was concentrated under reduced pressure to give 1 L solution. The solution was washed with brine (3×500 mL), dried over Na₂SO₄, filtered and the filtrate was concentrated under reduced pressure to give a residue The residue was triturated by PE/EA=10:1 (500 mL) and the solid was collected by suction filtration to give 2-hydroxy-4-(4-methylthiazol-5-yl)benzonitrile (D2) (80 g, yield 61.04%) as yellow solid.

¹H NMR (400 MHz, DMSO-d6) δ=11.32 (s, 1H), 9.07 (s, 1H), 7.70 (d, J=7.8 Hz, 1H), 7.13 (d, J=1.5 Hz, 1H), 7.07 (dd, J=1.7, 8.1 Hz, 1H), 2.49 (s, 3H)

### General Procedure for Preparation of 2-(aminomethyl)-5-(4-methylthiazol-5-yl)phenol (D3)-ET39710-106

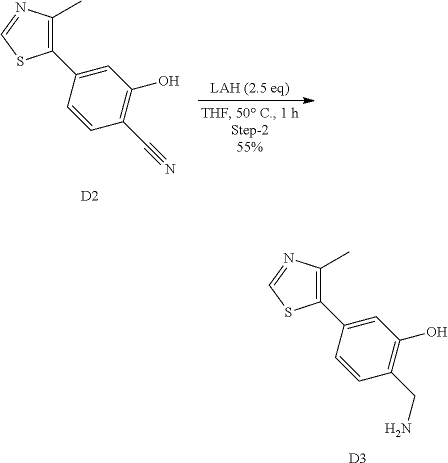

To a stirred solution of 2-hydroxy-4-(4-methylthiazol-5-yl)benzonitrile (D2) (80 g, 369.93 mmol) in THF (1 L) under an atmosphere of nitrogen was added LAH (35.10 g, 924.82 mmol) in several portions at 10° C. The resulting mixture was heated at 50° C. for 1 h. LCMS showed the reaction was completed. The mixture was cooled to 0° C., quenched by the H₂O (35 mL, added slowly and drop wise), 15% NaOH (aq.) (35 mL) and H₂O (105 mL). The solids precipitated were removed by suction filtration, the filtrate was concentrated under reduced pressure to give 2-(aminomethyl)-5-(4-methylthiazol-5-yl)phenol (D3) (45 g. yield 55.22%) as yellow solid.

¹H NMR (400 MHz, DMSO-d₆) δ=8.80 (s, 1H), 6.84 (d, J=7.3 Hz, 1H), 6.37 (s, 1H), 6.12 (dd, J=1.5, 7.3 Hz, 1H), 3.53 (s, 2H), 2.42 (s, 3H)

### General Procedure for Preparation of (2S,4R)-tert-butyl 4-hydroxy-2-((2-hydroxy-4-(4-methylthiazol-5-yl)benzyl)carbamoyl)pyrrolidine-1-carboxylate (D4)-ET39710-107

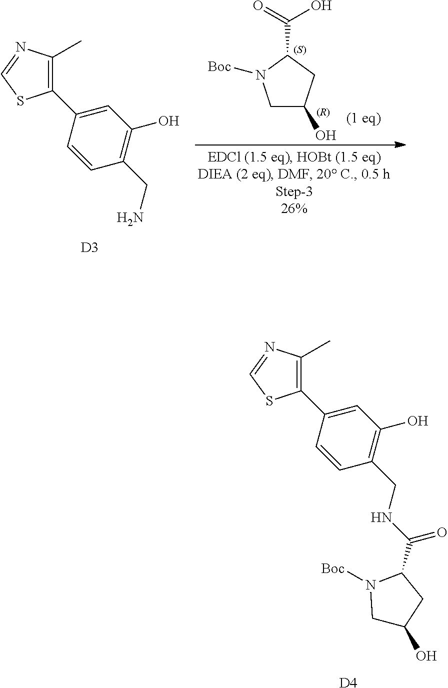

To a solution of 2-(aminomethyl)-5-(4-methylthiazol-5-yl)phenol (D3) (45 g, 204.28 mmol) in DMF (500 mL) were added (2S,4R)-1-(tert-butoxycarbonyl)-4-hydroxypyrrolidine-2-carboxylic acid (47.24 g, 204.28 mmol), DIEA (52.80 g, 408.55 mmol), HOBt (41.40 g, 306.41 mmol) and EDCI (47.57 g, 306.41 mmol) at 0° C. under N₂. The mixture was stirred at 20° C. for 0.5 h. LCMS showed the reaction was completed. The reaction mixture was poured into water (500 mL) and stirred for 5 min. The mixture was extracted with EA (3×800 mL). The combined organic layers were washed with brine (1 L), dried over Na₂SO₄, filtered and the filtrate was concentrated under reduced pressure to give a residue. The residue was purified by column chromatography on silica gel (eluting with PE/EA=20:1 to 0:1) to give (2S,4R)-tert-butyl 4-hydroxy-2-((2-hydroxy-4-(4-methylthiazol-5-yl)benzyl)carbamoyl)pyrrolidine-1-carboxylate (D4) (23.4 g. yield 26.42%) as yellow solid.

¹H NMR (400 MHz, DMSO-d₆) δ=9.92 (s, 1H), 8.96 (s, 1H), 8.54-8.30 (m, 1H), 7.27-7.15 (m, 1H), 6.91 (s, 1H), 6.89-6.81 (m, 1H), 4.32-4.11 (m, 4H), 3.48-3.34 (m, 3H), 2.44 (s, 3H), 2.13-1.98 (m, 1H), 1.93-1.81 (m, 1H), 1.41 (s, 3H), 1.26-1.20 (m, 6H)

### General Procedure for Preparation of (2S,4R)-4-hydroxy-N-(2-hydroxy-4-(4-methylthiazol-5-yl)benzyl)pyrrolidine-2-carboxamide hydrochloride (D5)-ET39710-112

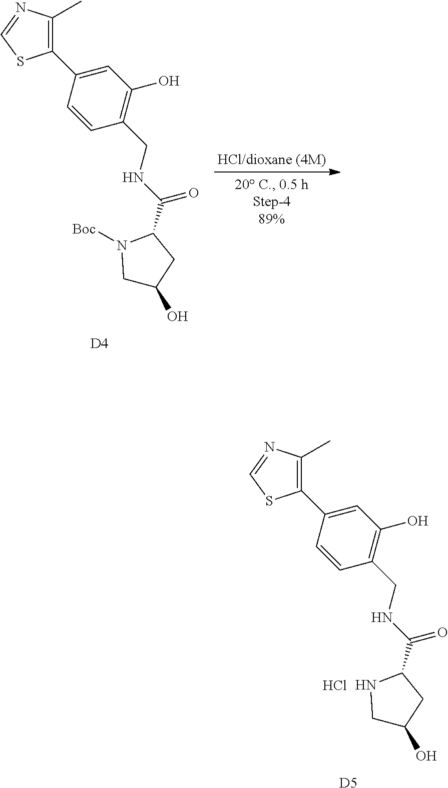 [SMILES: Cc1ncsc1-c1ccc(CNC(=O)[C@@H]2C[C@@H](O)CN2C(=O)OC(C)(C)C)c(O)c1 ; "D4" ; Cc1ncsc1-c1ccc(CNC(=O)[C@@H]2C[C@@H](O)CN2)c(O)c1 ; Cl ; "D5"]

A mixture of (2S,4R)-tert-butyl 4-hydroxy-2-((2-hydroxy-4-(4-methylthiazol-5-yl)benzyl)carbamoyl)pyrrolidine-1-carboxylate (D4) (23.4 g, 53.98 mmol) in HCl/dioxane (250 mL, 4 M) was stirred at 20° C. for 0.5 h. LCMS showed the reaction was completed. The mixture was concenrtated under reduced pressure to give (2S,4R)-4-hydroxy-N-(2-hydroxy-4-(4-methylthiazol-5-yl)benzyl)pyrrolidine-2-carboxamide hydrochloride (D5) (17.80 g, yield 89.16%, HCl salt) as white solid.

¹H NMR (400 MHz, DMSO-d₆) δ=10.17-9.86 (m, 2H), 9.05-8.96 (m, 2H), 8.66 (br d, J=1.9 Hz, 1H), 7.20 (d, J=7.6 Hz, 1H), 7.02 (d, J=1.4 Hz, 1H), 6.91 (dd, J=1.4, 7.6 Hz, 1H), 4.44 (br s, 1H), 4.41-4.34 (m, 1H), 4.31 (t, J=5.5 Hz, 2H), 3.40-3.28 (m, 1H), 3.15-3.03 (m, 1H), 2.46 (s, 3H), 2.37-2.27 (m, 1H), 1.96-1.87 (m, 1H)

### General Procedure for Preparation of tert-butyl ((S)-1-((2S,4R)-4-hydroxy-2-((2-hydroxy-4-(4-methylthiazol-5-yl)benzyl)carbamoyl) pyrrolidin-1-yl)-3,3-dimethyl-J-oxobutan-2-yl) carbamate (D6)-ET39710-113

To a solution of (2S,4R)-4-hydroxy-N-(2-hydroxy-4-(4-methylthiazol-5-yl)benzyl)pyrrolidine-2-carboxamide hydrochloride (D5) (16 g, 43.26 mmol) in DMF (160 mL) were added (2S)-2-(tert-butoxycarbonylamino)-3,3-dimethyl-butanoic acid (10.01 g, 43.26 mmol), DIEA (116.77 g, 129.78 mmol) and HATU (19.74 g, 51.91 mmol) at 0° C. The mixture was stirred at 20° C. for 1 h. LCMS showed the reaction was completed. The mixture was poured into ice-water (100 mL) and stirred for 5 min. The mixture was extracted with EA (3×150 mL).

The combined organic layers were washed with brine (300 mL), dried over Na₂SO₄, filtered and the filtrate was concentrated under reduced pressure to give a residue. The residue was purified by column chromatography on silica gel (eluting with PE/EA=50:1 to 0:1) to give tert-butyl ((S)-1-((2S,4R)-4-hydroxy-2-((2-hydroxy-4-(4-methylthiazol-5-yl)benzyl)carbamoyl) pyrrolidin-1-yl)-3,3-dimethyl-1-oxobutan-2-yl) carbamate (D6) (12.1 g. yield 51.17%) as yellow solid.

¹H NMR (400 MHz, MeOD-d₄) δ=8.85 (s, 1H), 8.74 (dd, J=1.4, 4.3 Hz, 1H), 8.43 (dd, J=1.2, 8.3 Hz, 1H), 7.52 (dd, J=4.8, 8.6 Hz, 1H), 7.36 (d, J=8.1 Hz, 1H), 6.92-6.87 (m, 2H), 4.60 (s, 1H), 4.50 (br s, 1H), 4.45-4.32 (m, 2H), 4.29 (s, 1H), 3.91-3.76 (m, 2H), 2.49-2.47 (m, 3H), 2.25-2.06 (m, 2H), 1.48-1.42 (m, 9H), 1.00 (s, 9H)

### General Procedure for Preparation of (2S,4R)-1-[(28)-2-amino-3,3-dimethyl-butanoyl]-4-hydroxy-N-[[2-hydroxy-4-(4-methylthiazol-5-yl)phenyl]methyl]pyrrolidine-2-carboxamide (D7)-ET39710-115

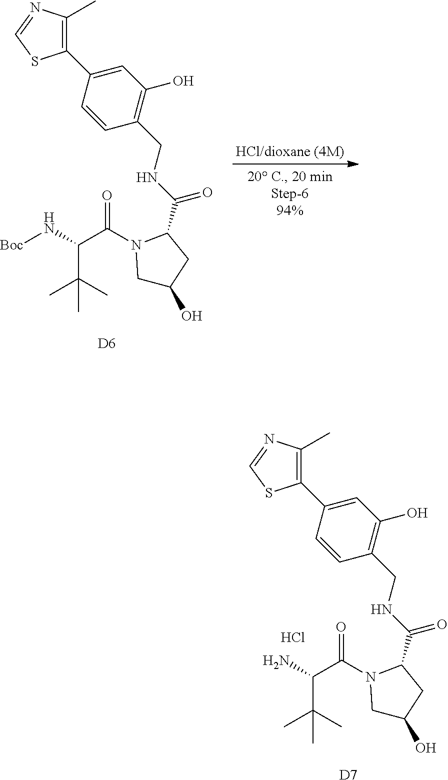

A mixture of tert-butyl ((S)-1-((2S,4R)-4-hydroxy-2-((2-hydroxy-4-(4-methylthiazol-5-yl)benzyl)carbamoyl) pyrrolidin-1-yl)-3,3-dimethyl-1-oxobutan-2-yl) carbamate (D6) (12 g, 21.95 mmol) in HCl/dioxane (120 mL, 4 M) was stirred at 20° C. for 20 min. LCMS showed the reaction was completed. The mixture was concenrtated under reduced pressure to give (2S,4R)-1-[(2S)-2-amino-3,3-dimethyl-butanoyl]-4-hydroxy-N-[[2-hydroxy-4-(4-methylthiazol-5-yl)phenyl]methyl]pyrrolidine-2-carboxamide (D7) (9.95 g, yield 93.84%, HCl salt) as white solid.

¹H NMR (400 MHz, DMSO-d₆) δ=9.05 (s, 1H), 8.63 (t, J=6.1 Hz, 1H), 8.15 (br d, J=3.9 Hz, 3H), 7.32 (d, J=7.8 Hz, 1H), 6.98 (d, J=2.0 Hz, 1H), 6.81 (dd, J=2.0, 7.8 Hz, 1H), 4.57 (t, J=8.3 Hz, 1H), 4.37 (br s, 1H), 4.33-4.23 (m, 1H), 4.20-4.10 (m, 1H), 3.90 (br d, J=4.9 Hz, 1H), 3.77 (br d, J=10.8 Hz, 1H), 3.64-3.51 (m, 1H), 2.12 (br dd, J=7.8, 12.7 Hz, 1H), 1.91 (s, 1H), 1.59 (s, 4H), 1.01 (s, 9H)

### General Procedure for Preparation of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (NUCC-0226278)-ET39710-118

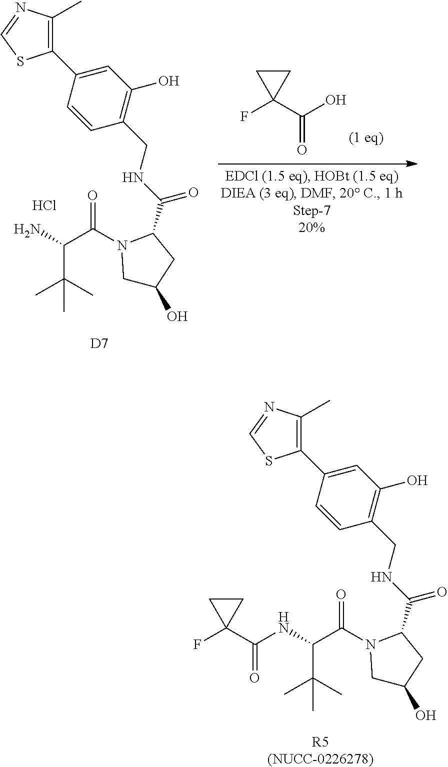

To a solution of 1-fluorocyclopropanecarboxylic acid (861.90 mg, 8.28 mmol) in DMF (40 mL) were added (2S,4R)-1-((S)-2-amino-3,3-dimethylbutanoyl)-4-hydroxy-N-(2-hydroxy-4-(4-methylthiazol-5-yl)benzyl)pyrrolidine-2-carboxamide hydrochloride (D7) (4 g, 8.28 mmol), HOBt (1.68 g, 12.42 mmol), DIEA (3.21 g, 24.84 mmol) and EDCI (1.93 g, 12.42 mmol) at 0° C. The mixture was stirred at 20° C. for 1 b. LCMS showed the reaction was completed. The reaction mixture was poured into water (40 mL) and stirred for 5 min. The mixture was extracted with EA (3×50 mL). The combined organic layers were washed with brine (100 mL), dried over Na₂SO₄, filtered and the filtrate was concentrated under reduced pressure to give a residue. The residue was purified by prep-HPLC (HCl condition) to give R⁵ (0.9 g, yield 20.40%) as white solid.

$$LCMS ( ESI + ) : m / z = 533. ( M + H ) + , RT : 0.832 min .$$

¹H NMR (400 MHz, DMSO-d6) δ=9.97-9.71 (m, 1H), 9.09-8.94 (m, 1H), 8.66-8.45 (m, 1H), 7.36-7.23 (m, 2H), 7.01-6.87 (m, 1H), 6.87-6.80 (m, 1H), 4.70-4.43 (m, 6H), 4.32-4.03 (m, 3H), 3.69-3.56 (m, 2H), 2.13-2.03 (m, 1H), 1.96-1.87 (m, 1H), 1.44-1.30 (m, 2H), 1.27-1.17 (m, 2H), 1.01-0.89 (m, 9H)

### Prep-HPLC Method:

Instrument: Shimadzu LC-8A preparative HPLC
Column: Phenomenex luna C18 250*50 mm*10 um
Mobile phase: A for H₂O=(0.04% HCl) and B for CH₃CN
Gradient: B from 25% to 45% in 20 min
Flow rate: 80 mL/min
Wavelength: 220&254 nm

SFC method:
Column: Chiralpak AD-3, 50 × 4.6 mm I.D., 3 um
Mobile phase: A: CO₂ B: EtOH (0.1% IPAm, v/v)
Gradient:

| Time | A % | B % |
|---|---|---|
| 0.0 | 95 | 5 |
| 0.2 | 95 | 5 |
| 1.2 | 50 | 50 |
| 2.2 | 50 | 50 |
| 2.6 | 95 | 5 |
| 3.0 | 95 | 5 |

Flow rate: 3.4 mL/min
Column temp.: 35° C.
ABPR: 1800 psi

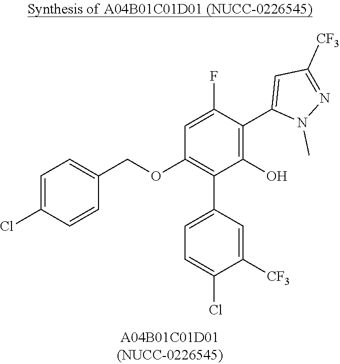 [SMILES: Cn1nc(C(F)(F)F)cc1-c1c(F)cc(OCc2ccc(Cl)cc2)c(-c2ccc(Cl)c(C(F)(F)F)c2)c1O]

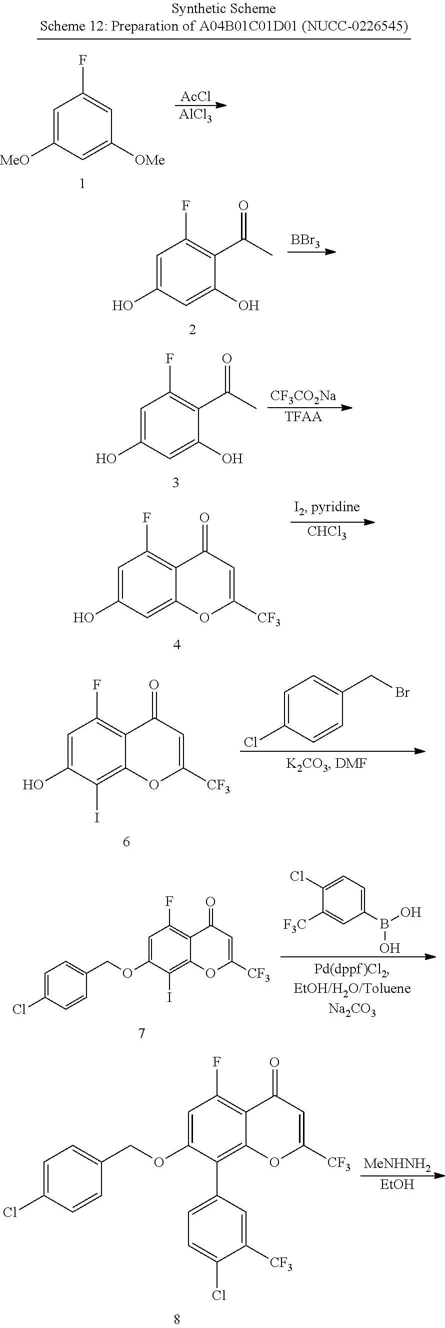

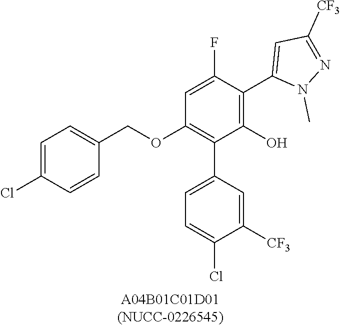 [SMILES: Cn1nc(C(F)(F)F)cc1-c1c(F)cc(OCc2ccc(Cl)cc2)c(-c2ccc(Cl)c(C(F)(F)F)c2)c1O]

### Chemical Synthesis

### LCMS Methods:

### Method 1: 5_95AB_2_min

LC/MS (The column used for chromatography was a Chromolith RP-18e 25-2 mm. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95-100% B (0.70-1.15 min), 5% B in 1.16 min with a hold at 5% B for 0.34 min. The flow rate was 1.5 mL/min.

### Method 2: 50_100 CD_6min-220-254-ELSD

LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was acetonitrile. The column used for chromatography was a Xbridge Shield RP18 2.1*50 mm column (Sum particles). Detection methods are diode array (DAD) and evaporative light scattering (ELSD) detection as well as positive electrospray ionization. MS range was 100-1000.

### Experimentals for Largest Scale Run

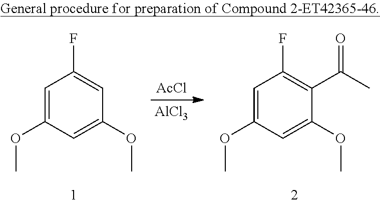

To a solution of AlCl₃ (5.12 g, 38.42 mmol, 2.10 mL, 1.2 eq) in DCM (50 mL) was added acetyl chloride (3.77 g, 48.03 mmol, 3.43 mL, 1.5 eq) dropwise at 5-10° C., the reaction was stirred at 5° C. for 15 mins, then Compound 1 (5 g, 32.02 mmol, 1 eq) was added dropwise at 0-10° C. and the reaction was stirred at 20° C. for 6 hrs. TLC (petroleum ether/ethyl acetate=3/1) showed the starting material was consumed and two new peaks were generated. The reaction was poured into 200 mL of ice-water. The aqueous layer is extracted with dichloromethane (2×100 mL). The combined organic phases are washed with water (100 mL), dried over sodium sulfate and concentrated under reduced pressure to give the crude product. The crude product was purified by column chromatography on silica gel (eluted with petroleum ether/ethyl acetate=50/1 to 10/1) to give Compound 2 (4.5 g, yield 67.37%) as a colorless oil.

¹H NMR (ET42365-46-P1A, 400 MHz, CHLOROFORM-d) δ 2.52 (d, J=1.63 Hz, 3H), 3.82 (s, 3H), 3.84 (s, 3H), 6.22-6.28 (m, 2H)

¹⁹F NMR (ET42365-46-P1A, 400 MHz, CHLOROFORM-d) δ−112.015

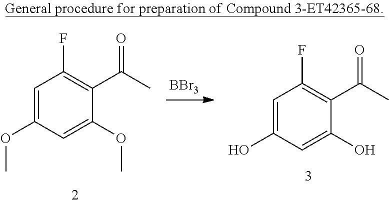

To a solution of Compound 2 (4 g, 20.18 mmol, 1 eq) in DCM (80 mL) was added BBr₃ (20.22 g, 80.73 mmol, 7.78 mL, 4 eq) dropwise at −30° C. the reaction was stirred at 20° C. for 24 hrs. LCMS showed the starting material was consumed and a new peak with desired product Ms was detected. The reaction was poured into 100 mL of ice-water. The aqueous layer is extracted with dichloromethane (2×50 mL). The combined organic phases are washed with brine (50 mL), dried over Na₂SO₄, filtered and concentrated under reduced pressure to give crude product. The crude product was purified by column chromatography on silica gel (eluted with petroleum ether/ethyl acetate=30/1 to 10/1) to give Compound 3 (2.3 g. yield 63.63%) as a white solid.

¹H NMR (ET42365-68-P1H, 400 MHz, DMSO-d6) δ 2.53 (d, J=6.80 Hz, 3H), 6.11 (d, J=2.19 Hz, 1H), 6.19 (dd, J=14.03, 2.19 Hz, 1H), 11.06 (s, 1H), 13.05 (s, 1H)

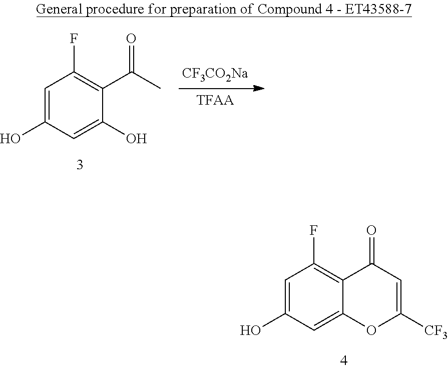

A suspension of Compound 3 (2 g, 11.76 mmol, 1 eq) in TFAA (20 mL) was placed in a high pressure tube (30 mL). Sodium trifluoroacetate (3.52 g, 25.86 mmol, 2.2 eq) was added and the system was capped and stirred at 130° C. for 24 hrs. LCMS showed all starting material was consumed. The reaction was cooled to 25° C. and neutralized by saturated aqueous K₂CO₃ solution until no more bubbling was observed. The organic layer was separated and the aqueous portion was extracted with ethyl acetate (3×150 mL). The combined organic layers were washed with brine (100 mL), dried over anhydrous Na₂SO₄, and concentrated under reduced pressure. The crude product was purified by column chromatography on silica, eluted with petroleum ether/ethyl acetate=50/1 to 5/1 to give Compound 4 (0.48 g, yield 10.5%) as a light yellow solid.

$$LCMS ( ESI + ) : RT = 0.773 min , m / z 249. {( M + H )}^{+} .$$

5-95AB_2_min: LC/MS (The column used for chromatography was a Chromolith RP-18e 25-2 mm. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95-100% B (0.70-1.15 min), 5% B in 1.16 min with a hold at 5% B for 0.34 min. The flow rate was 1.5 mL/min.

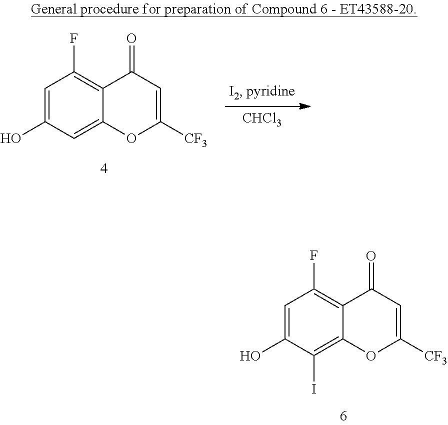 [SMILES: O=c1cc(C(F)(F)F)oc2cc(O)cc(F)c12 ; "CHCl3" ; "I2, pyridine" ; O=c1cc(C(F)(F)F)oc2c(I)c(O)cc(F)c12]

To a solution of Compound 4 (0.43 g, 1.73 mmol, 1 eq) in CHCl₃ (9 mL) were added I₂ (1.76 g, 6.93 mmol, 4 eq) and pyridine (548.31 mg, 6.93 mmol, 559.50 μL, 4 eq) at 25° C. The reaction was stirred for 2 hrs at 25° C. LCMS showed the reaction was completed. The reaction mixture was poured into water (20 mL) and extracted with ethyl acetate (3×20 ml). The combined organic layers were washed with brine (20 mL), dried over Na₂SO₄, filtered and the filtrate was concentrated under reduced pressure. The crude product was purified by column chromatography on silica, eluted with petroleum ether/ethyl acetate=10/1 to 2/1 to give Compound 6 (0.38 g, yield 45.5%) as a brown solid.

¹H NMR (ET43588-20-P1H, 400 MHz, MeOD) δ 6.72-6.80 (m, 2H)

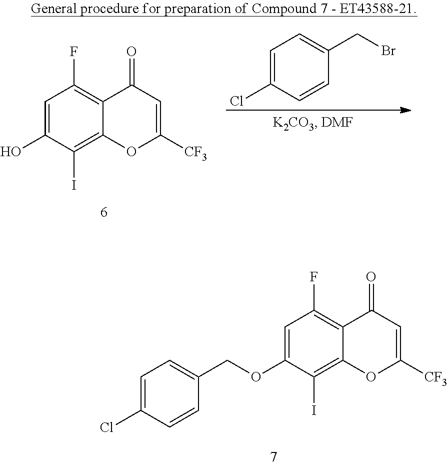

A solution of Compound 6 (0.38 g, 1.02 mmol) in DMF (3.5 mL) was added 1-(bromomethyl)-4-chloro-benzene (250.51 mg, 1.22 mmol) and K₂CO₃ (280.83 mg, 2.03 mmol, 2 eq) at 25° C. The reaction mixture was stirred and heated at 80° C. for 2 hrs. LCMS showed the reaction was completed. After cooling to room temperature, the mixture was poured into water (20 mL) and extracted with ethyl acetate (3×20 mL). The combined organic layers were dried over Na₂SO₄ and concentrated under reduced pressure. The crude product was purified by column chromatography on silica, eluted with petroleum ether/ethyl acetate=5/1 to 3/1 to give Compound 7 (0.5 g, yield 80.0%) as a brown solid.

¹H NMR (ET43588-20-P1H, 400 MHz, CDCl₃) δ 5.25 (s, 2H) 6.66 (s, 1H) 6.77 (d, J=12.17 Hz, 1H) 7.39-7.48 (m, 4H).

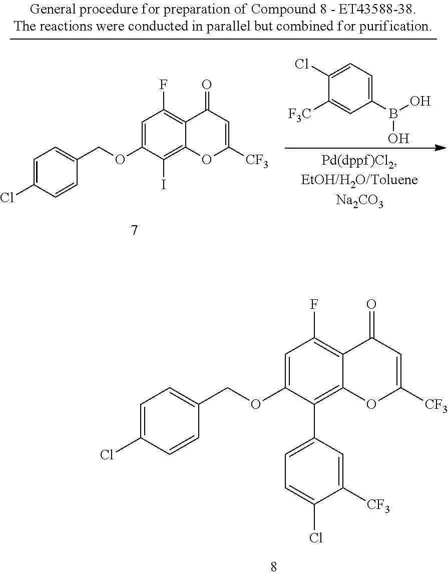 [SMILES: O=c1cc(C(F)(F)F)oc2c(I)c(OCc3ccc(Cl)cc3)cc(F)c12 ; OB(O)c1ccc(Cl)c(C(F)(F)F)c1 ; O=c1cc(C(F)(F)F)oc2c(-c3ccc(Cl)c(C(F)(F)F)c3)c(OCc3ccc(Cl)cc3)cc(F)c12]

To a solution of Compound 7 (0.28 g, 748.61 μmol) in toluene (67.2 mL), ethanol (13.44 mL) and water (2.688 mL) were added [4-chloro-3-(trifluoromethyl)phenyl]boronic acid (167.97 mg, 748.61 μmol), Na₂CO₃ (487.82 mg, 1.50 mmol) and Pd(dppf)Cl₂ (54.78 mg, 74.86 μmol) at 25° C. under N₂ atmosphere. The reaction mixture was stirred and heated at 80° C. for 2 hrs under N₂ atmosphere. LCMS showed the reaction was completed. Additional two reactions were set up as described above and combined for purification. The reaction mixtures were poured into water (30 mL) and extracted with ethyl acetate (3×30 mL). The combined organic layers were dried over Na₂SO₄, filtered and concentrated under reduced pressure. The crude product was by re-crystallization from ethyl acetate/petroleum ether (10/1, 3 mL) to give Compound 8 (0.21 g, yield 26.7%) as a brown solid.

¹H NMR (ET43588-38-PH1, 400 MHz, CDCl₃) δ ppm 5.15 (s, 2H) 6.63 (s, 1H) 6.89 (d, J=12.13 Hz, 1H) 7.19 (d, J=8.38 Hz, 2H) 7.35 (d, J=8.50 Hz, 2H) 7.51 (dd, J=8.38, 1.88 Hz, 1H) 7.61 (d, J=8.25 Hz, 1H) 7.77 (d, J=1.88 Hz, 1H).

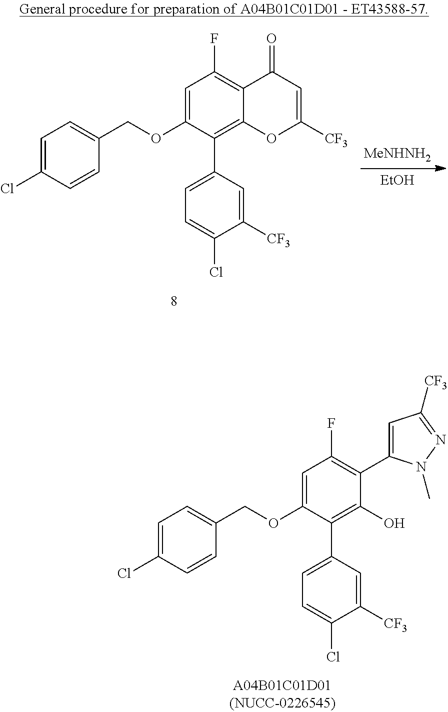 [SMILES: "MeNHNH2" ; "EtOH" ; O=c1cc(C(F)(F)F)oc2c(-c3ccc(Cl)c(C(F)(F)F)c3)c(OCc3ccc(Cl)cc3)cc(F)c12 ; Cn1nc(C(F)(F)F)cc1-c1c(F)cc(OCc2ccc(Cl)cc2)c(-c2ccc(Cl)c(C(F)(F)F)c2)c1O]

To a suspension of Compound 8 (0.16 g, 290.26 μmol) in ethanol (1.6 mL) was added and CH₃NHNH₂ (200.59 mg, 1.74 mmol, 229.24 μL, 40% purity) at 25° C. Then the reaction mixture was stirred and heated at 80° C. for 4 hrs. LCMS showed the reaction was complete. The reaction mixture was concentrated under reduced pressure. The residue was purified by prep-HPLC to give A04B01C01D01 (17.7 mg, yield 10.5%) as a white solid.

¹H NMR (ET43588-57-P1H1, 400 MHz, CDCl₃) δ 3.85 (s, 3H) 5.04 (s, 2H) 5.27 (s, 1H) 6.53 (d, J=11.21 Hz, 1H) 6.65 (s, 1H) 7.17 (d, J=8.23 Hz, 2H) 7.34 (d, J=8.34 Hz, 2H) 7.48-7.55 (m, 1H) 7.57-7.64 (m, 1H) 7.77 (s, 1H).\

Preparative HPLC method:
Instrument: Gilson 281 semi-preparative HPLC system
Mobile phase: A: 10 mM NH₄HCO₃ in H₂O; B: CAN
Column: Waters Xbridge BEH C18 100 * 25 mm * 5 um
Flow rate: 25 mL/min
Monitor wavelength: 220 & 254 nm

| Time | B % |
|---|---|
| 0.0 | 78 |
| 10.0 | 98 |
| 10.1 | 98 |
| 10.2 | 100 |
| 12.2 | 100 |
| 12.3 | 78 |
| 13.5 | 78 |

$$LCMS ( ESI + ) : 2.919 min , m / z = 579.1 {( M + H )}^{+} .$$

### 50_100 CD_6min-220-254-ELSD

LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was acetonitrile. The column used for chromatography was a Xbridge Shield RP18 2.150 mm column (5 um particles). Detection methods are diode array (DAD) and evaporative light scattering (ELSD) detection as well as positive electrospray ionization. MS range was 100-1000.

### Synthesis of NUCC-0226530 (ET42365-98-1), NUCC-0226528 (ET42365-86-1) and NUCC-0226520 (ET42365-63-1)

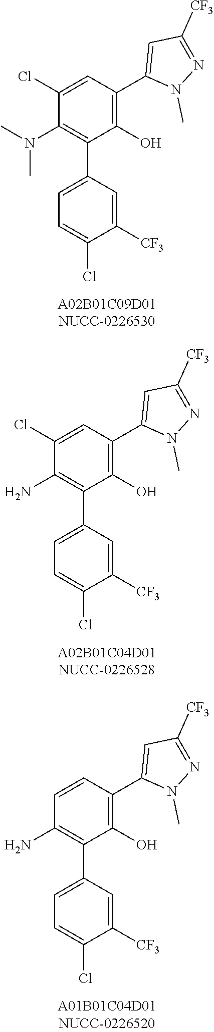 [SMILES: CN(C)c1c(Cl)cc(-c2cc(C(F)(F)F)nn2C)c(O)c1-c1ccc(Cl)c(C(F)(F)F)c1 ; Cn1nc(C(F)(F)F)cc1-c1cc(Cl)c(N)c(-c2ccc(Cl)c(C(F)(F)F)c2)c1O ; Cn1nc(C(F)(F)F)cc1-c1ccc(N)c(-c2ccc(Cl)c(C(F)(F)F)c2)c1O]

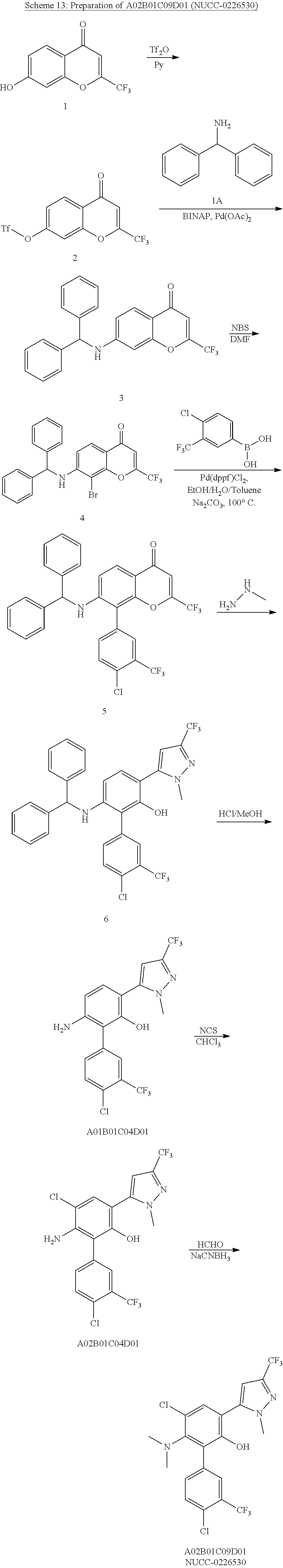

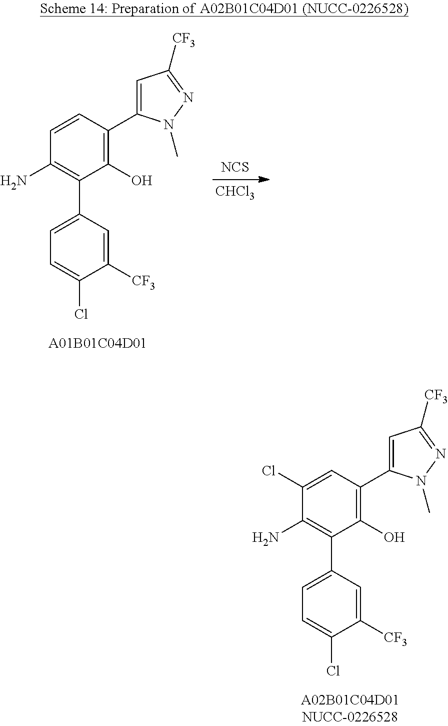

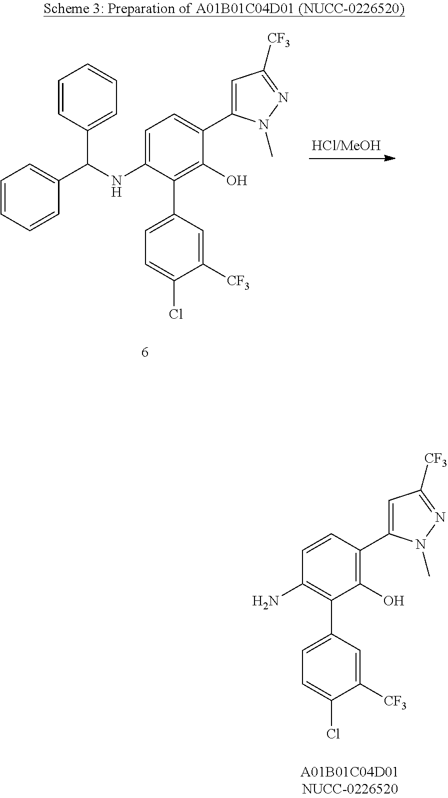 [SMILES: Cn1nc(C(F)(F)F)cc1-c1ccc(NC(c2ccccc2)c2ccccc2)c(-c2ccc(Cl)c(C(F)(F)F)c2)c1O ; Cn1nc(C(F)(F)F)cc1-c1ccc(N)c(-c2ccc(Cl)c(C(F)(F)F)c2)c1O]

### Chemical Synthesis

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (Sum particles). Detection methods are diode array (DAD) and evaporative light scattering (ELSD) detection. MS mode was positive electrospray ionization. MS range was 100-1000.

5_95CD_6min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge C18 2.1*50 mm column (Sum particles). Detection methods are diode array (DAD) detection. MS mode was positive electrospray ionization. MS range was 100-1000.

### Experimentals for Largest Scale Run

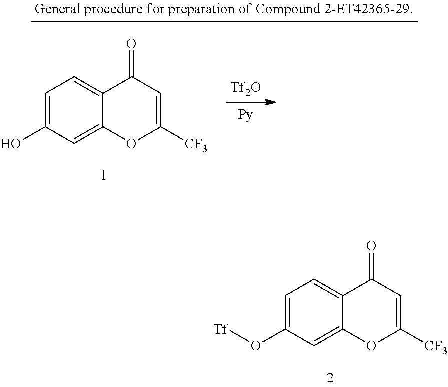

To a solution of Compound 1 (10 g, 43.45 mmol, 1 eq) and pyridine (13.75 g, 173.81 mmol, 14.03 mL, 4 eq) in DCM (150 ml) was added Tf₂O (18.39 g, 65.18 mmol, 10.75 mL, 1.5 eq) dropwise at 0° C., the reaction was stirred at 20° C. for 12 hrs. LCMS showed the starting material was consumed and a new peak with desired product Ms was detected. The reaction was diluted with water (300 mL) and extracted with DCM (3×100 mL). The organic layer was separated and the combined organic layer was washed with brine (150 mL), dried over Na₂SO₄, filtered and concentrated under reduced pressure to crude product. The crude product was purified by column chromatography on silica gel (eluted with petroleum ether/ethyl acetate=50/1 to 10/1) to give Compound 2 (12 g. yield 72.43%) as a white solid.

¹H NMR (ET42365-29-P1A, 400 MHz, CHLOROFORM-d) δ 6.79 (s, 1H), 7.42 (dd, J=8.88, 2.25 Hz, 1H), 7.56 (d, J=2.25 Hz, 1H), 8.34 (d, J=8.88 Hz, 1H)

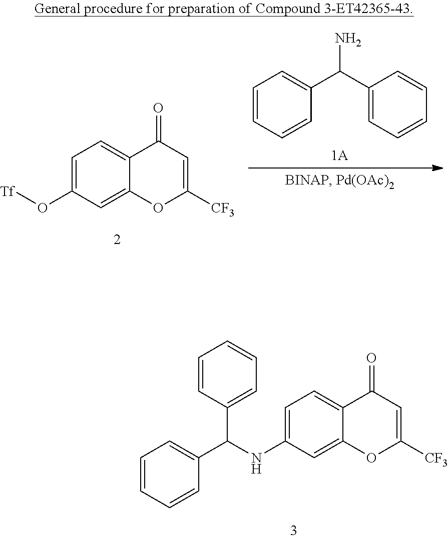

To a solution of Compound 2 (12 g, 33.13 mmol, 1 eq), Cs₂CO₃ (21.59 g, 66.26 mmol, 2 eq) and diphenylmethanamine (9.11 g, 49.70 mmol, 8.59 mL, 1.5 eq) in THF (300 mL) was added Pd(OAc)₂ (743.81 mg, 3.31 mmol, 0.1 eq) and BINAP (4.13 g, 6.63 mmol, 0.2 eq) under nitrogen, the reaction was stirred at 60° C. for 12 hrs. LCMS showed the starting material was consumed and a new peak with desired product Ms was detected. The reaction was filtered and the filtrate was concentrated to give the crude product. The crude product was purified by column chromatography on silica gel (eluted with petroleum ether/ethyl acetate=50/1 to 5/1) to give Compound 3 (13 g, yield 79.4%) as a yellow solid.

$$LCMS ( ESI + ) : RT = 0.827 min , m / z 396.2 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min. 5-95% B (0.01-0.70 min), 95% B (0.70-1.16 min), 95-5% B (1.16-1.50 min). The flow rate was 1.5 ml/min.

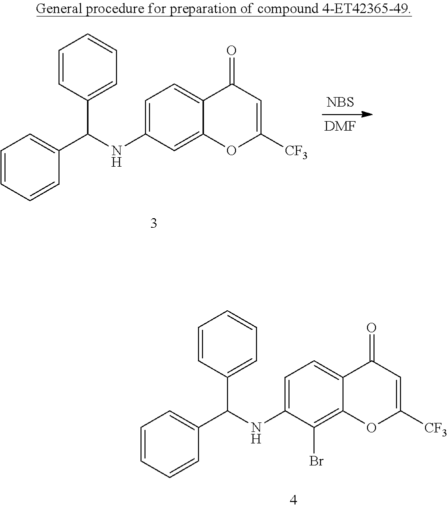

To a solution of Compound 3 (13 g, 26.30 mmol, 80% purity, 1 eq) in DMF (150 mL) was added NBS (5.62 g, 31.57 mmol, 1.2 eq) in portions at 0° C., the reaction was stirred at 25° C. for 16 hrs. LCMS showed the starting material was consumed and a new peak with desired product Ms was detected. The reaction was diluted with water (300 mL) and extracted with ethyl acetate (3×150 mL). The organic layer was separated and the combined organic layer was washed with brine (200 mL), dried over Na₂SO₄, filtered and concentrated under reduced pressure to crude product. The crude product was purified by column chromatography on silica gel (eluted with petroleum ether/ethyl acetate=60/1 to 10/1) to give Compound 4 (9 g, yield 68.54%) as a yellow solid.

¹H NMR (ET42365-49-P1A, 400 MHz, CHLOROFORM-d) δ 5.70 (br d, J=5.04 Hz, 1H), 5.77 (d, J=5.48 Hz, 1H), 6.60-6.66 (m, 2H), 7.29-7.43 (m, 10H), 7.88 (d, J=8.99 Hz, 1H)

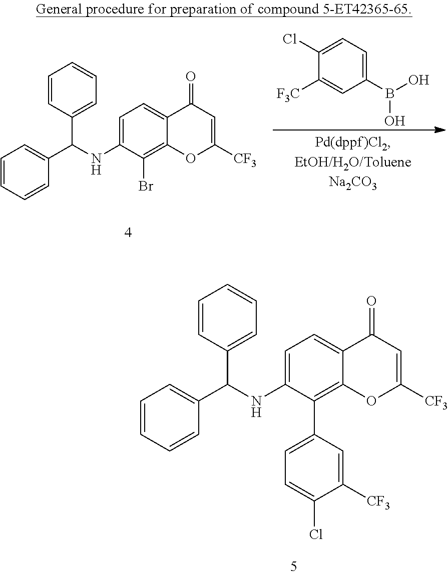 [SMILES: O=c1cc(C(F)(F)F)oc2c(Br)c(NC(c3ccccc3)c3ccccc3)ccc12 ; OB(O)c1ccc(Cl)c(C(F)(F)F)c1 ; O=c1cc(C(F)(F)F)oc2c(-c3ccc(Cl)c(C(F)(F)F)c3)c(NC(c3ccccc3)c3ccccc3)ccc12]

To a solution of Compound 4 (1 g, 2.11 mmol, 1 eq), [4-fluoro-3-(trifluoromethyl)-phenyl]boronic acid (613.75 mg, 2.95 mmol, 1.4 eq) and Na₂CO₃ (446.96 mg, 4.22 mmol, 2 eq) in a mixture solution of toluene (20 mL), EtOH (5 mL) and H₂O (1.2 mL) was added Pd(dppf)Cl₂·CH₂Cl₂ (172.19 mg, 210.85 μmol, 0.1 eq) under nitrogen, the reaction was stirred at 100° C. for 40 hrs. LCMS showed the starting material was consumed and a new peak with desired Ms was detected. The reaction was filtered and the filtrate was concentrated to give the crude product. The crude product was purified by column chromatography on silica gel (eluted with petroleum ether/ethyl acetate=50/1 to 10/1) to give Compound 5 (1.5 g, yield 37.19%) as a white solid.

$$LCMS ( ESI + ) : RT = 0.943 min , m / z 574.3 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

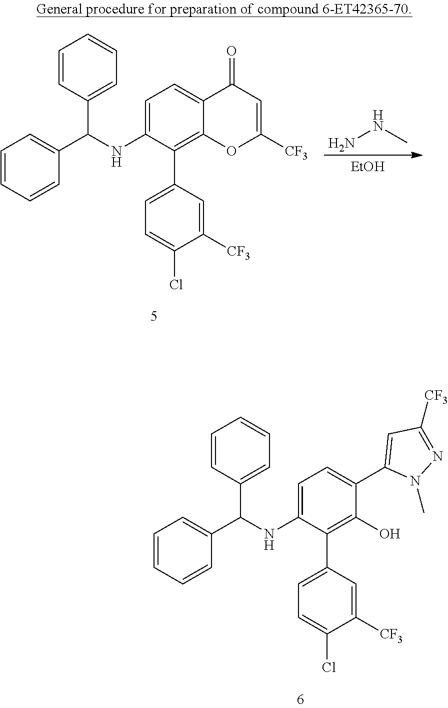

To a solution of Compound 5 (1.5 g, 2.61 mmol, 1 eq) in EtOH (30 mL) was added CH₃NHNH₂ (4.04 g, 35.08 mmol, 4.62 mL, 40% purity, 13.42 eq), the reaction was stirred at 80° C. for 12 hrs. LCMS showed the starting material was consumed and a new peak with desired product Ms was detected. The reaction was concentrated to give crude product. The crude product was purified by column chromatography on silica gel (eluted with petroleum ether/ethyl acetate=60/1 to 6/1) to give Compound 6 (1 g, yield 57.2%) as a yellow solid.

$$LCMS ( ESI + ) : RT = 0.943 min , m / z 602.3 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

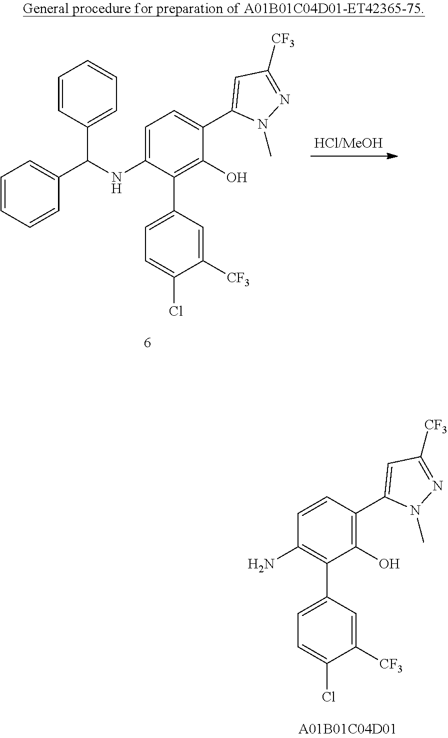 [SMILES: Cn1nc(C(F)(F)F)cc1-c1ccc(NC(c2ccccc2)c2ccccc2)c(-c2ccc(Cl)c(C(F)(F)F)c2)c1O ; Cn1nc(C(F)(F)F)cc1-c1ccc(N)c(-c2ccc(Cl)c(C(F)(F)F)c2)c1O]

To a solution of Compound 6 (0.5 g, 830.61 μmol, 1 eq) in MeOH (10 mL) was added con. HCl (2.52 g, 24.92 mmol, 36% purity, 30 eq), the reaction was stirred at 80° C. for 12 hrs. LCMS showed about 18% of the starting material was remaining and a new peak with desired product Ms was detected. The reaction was concentrated to give crude product. The crude product was purified by chromatography on silica gel (eluted with petroleum ether/ethyl acetate=50/1 to 5/1) to give A01B01C04D01 (550 mg, yield 68.38%) was obtained as a yellow solid.

$$LCMS ( ESI + ) : RT = 0.8 min , m / z 436.2 {( M + H )}^{+} .$$

5-95AB 2 min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

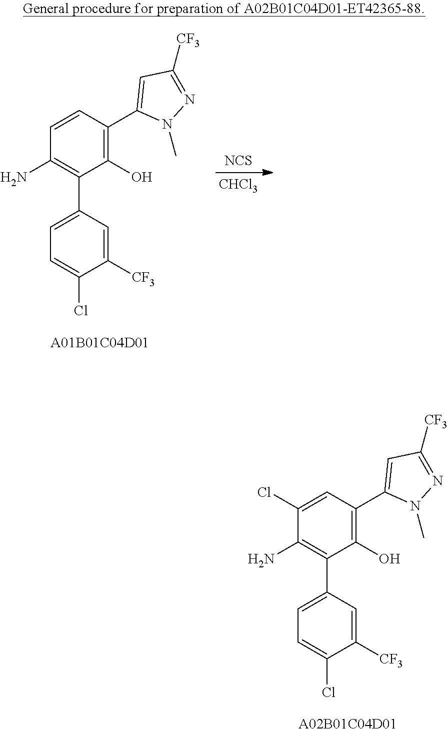

To a solution of A01B01C04D01 (200 mg, 458.98 μmol, 1 eq) in CHCl₃ (8 mL) was added NCS (73.55 mg, 550.77 μmol, 1.2 eq). The mixture was stirred at 20° C. for 12 hrs. TLC (petroleum ether/ethyl acetate=3/1) showed the starting material was consumed and a new spot was generated. The reaction was concentrated to give crude product. The crude product was purified by Prep-TLC (petroleum ether/ethyl acetate=3/1) to give A02B01C04D01 (90 mg, yield 40.7%) as a yellow solid.

$$LCMS ( ESI + ) : RT = 0.833 min , m / z 740.1 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

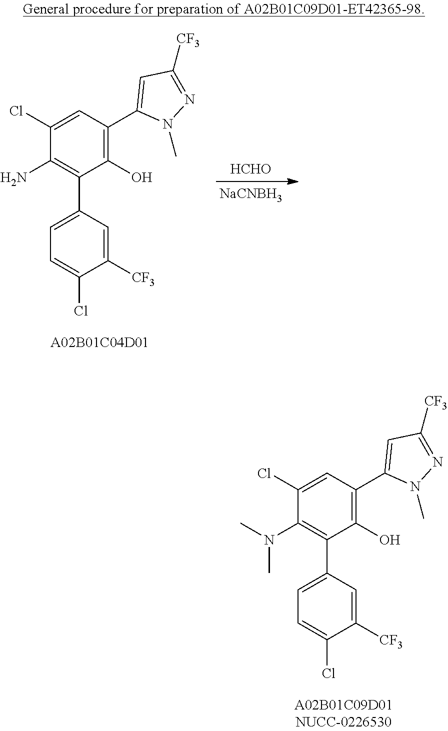

To a solution of A02B01C04D01 (90 mg, 191.41 μmol, 1 eq) in AcOH (2 mL) was added paraformaldehyde (57.47 mg, 1.91 mmol, 10 eq). The mixture was stirred at 20° C. for 1.5 hrs, then NaBH₃CN (60.14 mg, 957.05 μmol, 5 eq) was added and the mixture was stirred at 25° C. for 12 hrs. TLC (petroleum ether/ethyl acetate=4/1) showed the starting material was consumed and a new spot was generated. The reaction was concentrated to give crude product. The crude product was purified by Prep-TLC (petroleum ether/ethyl acetate=4/1) to give A02B01C09D01 (41.9 mg, yield 42.57%) as off-white solid.

¹H NMR (ET42365-98-P1, 400 MHz, CHLOROFORM-d) δ 2.53 (s, 6H), 3.79 (s, 3H), 4.86 (s, 1H), 6.50 (s, 1H), 7.20 (s, 1H), 7.37 (dd, J=8.19, 1.94 Hz, 1H), 7.55-7.61 (m, 2H)

$$LCMS ( ESI + ) : RT = 2.94 min , m / z 498.1 {( M + H )}^{+} .$$

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (5 um particles). Detection methods are diode array (DAD) and evaporative light scattering (ELSD) detection. MS mode was positive electrospray ionization. MS range was 100-1000.

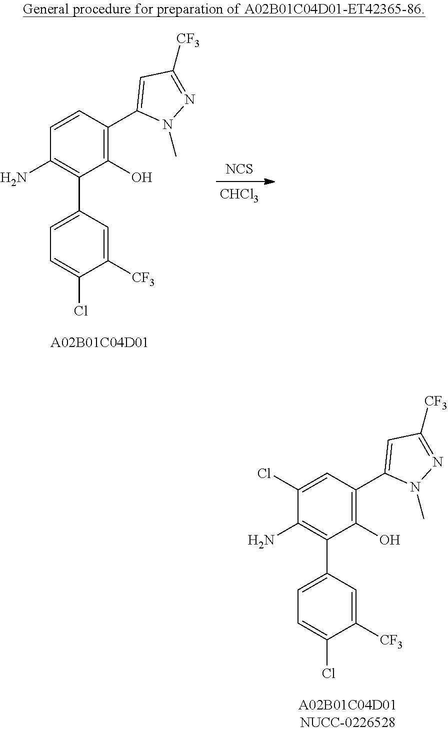

To a solution of A01B01C04D01 (150 mg, 344.23 μmol, 1 eq) in CHCl₃ (6 mL) was added NCS (68.95 mg, 516.35 μmol, 1.5 eq) in portions at 0° C., the reaction was stirred at 20° C. for 48 hrs. LCMS showed the starting material was consumed and a new peak with desired product Ms was detected. The reaction was concentrated to give crude product. The crude product was purified by Prep-TLC (petroleum ether/ethyl acetate=3/1) to give A02B01C04D01 (26.5 mg, yield 16.27%) as a yellow solid.

¹H NMR (ET42365-86-PIB, 400 MHz, CHLOROFORM-d) δ 3.86 (s, 3H), 4.17 (br s, 2H), 4.82 (s, 1H), 6.56 (s, 1H), 7.20 (s, 1H), 7.53-7.57 (m, 1H), 7.73 (d, J=8.28 Hz, 1H), 7.76 (d, J=1.63 Hz, 1H)

$$LCMS ( ESI + ) : RT = 3.527 min , m / z 470.1 {( M + H )}^{+} .$$

5_95CD_6min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge C18 2.1*50 mm column (Sum particles). Detection methods are diode array (DAD) detection. MS mode was positive electrospray ionization. MS range was 100-1000.

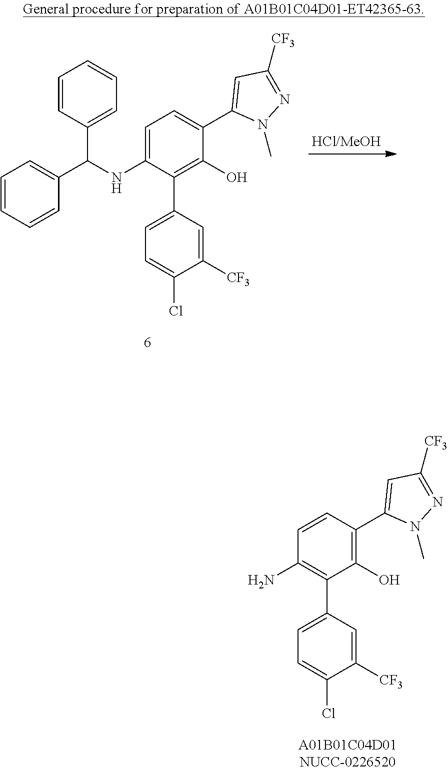 [SMILES: Cn1nc(C(F)(F)F)cc1-c1ccc(NC(c2ccccc2)c2ccccc2)c(-c2ccc(Cl)c(C(F)(F)F)c2)c1O ; Cn1nc(C(F)(F)F)cc1-c1ccc(N)c(-c2ccc(Cl)c(C(F)(F)F)c2)c1O]

To a solution of Compound 6 (100 mg, 166.12 μmol, 1 eq) in MeOH (1 mL) was added con. HCl (504.75 mg, 4.98 mmol, 36% purity, 30 eq), the reaction was stirred at 80° C. for 8 hrs. LCMS showed the starting material was consumed and a new peak with desired product Ms was detected. The reaction was concentrated to give crude product. The crude product was purified by Prep-HPLC (column: Phenomenex Gemini-NX C18 75*30 mm*3 um; mobile phase: [water (10 mM NH₄HCO₃)-ACN]; B %: 45%-65%, 8 min) to give A01B01C04D01 (21.5 mg, yield 29.61%) as a pink solid.

¹H NMR (ET42365-63-P1B, 400 MHz, CHLOROFORM-d) δ 3.76 (br s, 2H), 3.85 (s, 3H), 4.87 (s, 1H), 6.48 (d, J=8.25 Hz, 1H), 6.55 (s, 1H), 7.05 (d, J=8.25 Hz, 1H), 7.56 (dd, J=8.19, 1.81 Hz, 1H), 7.69 (d, J=8.25 Hz, 1H), 7.78 (d, J=1.63 Hz, 1H)

$$LCMS ( ESI + ) : RT = 3.311 min , m / z 436.1 {( M + H )}^{+} .$$

5_95CD_6min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge C18 2.1*50 mm column (5 um particles). Detection methods are diode array (DAD) detection. MS mode was positive electrospray ionization. MS range was 100-1000.

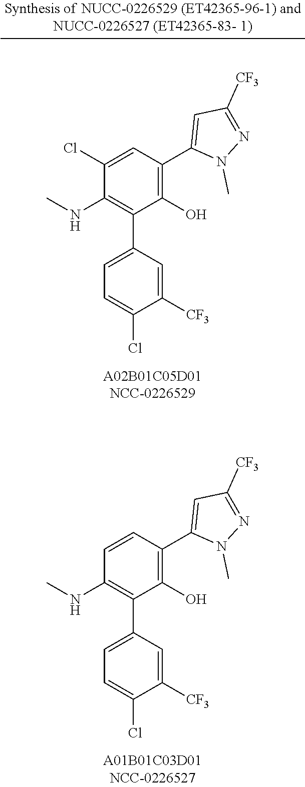 [SMILES: CNc1c(Cl)cc(-c2cc(C(F)(F)F)nn2C)c(O)c1-c1ccc(Cl)c(C(F)(F)F)c1 ; CNc1ccc(-c2cc(C(F)(F)F)nn2C)c(O)c1-c1ccc(Cl)c(C(F)(F)F)c1]

### Synthetic Scheme

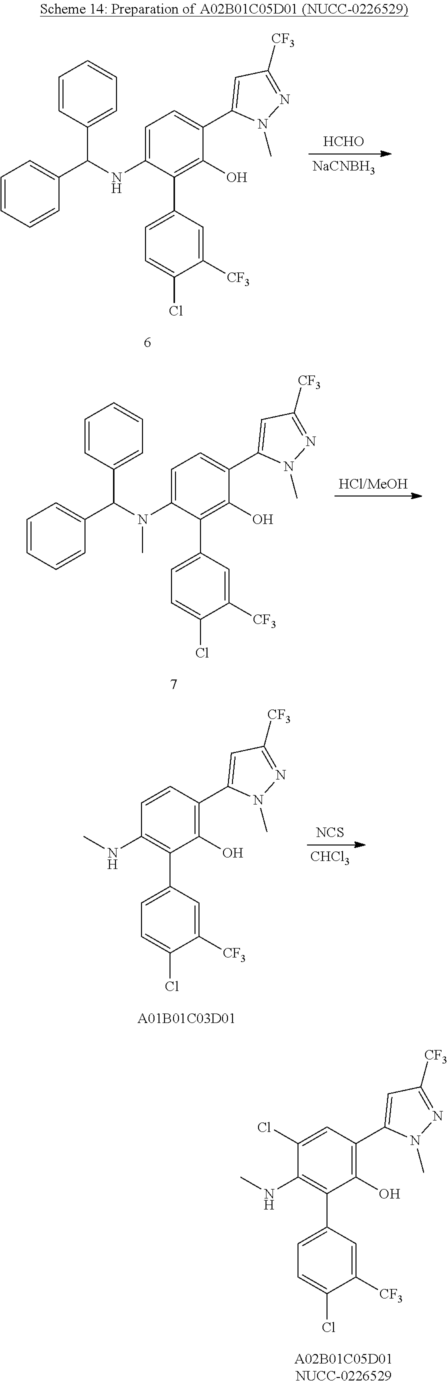

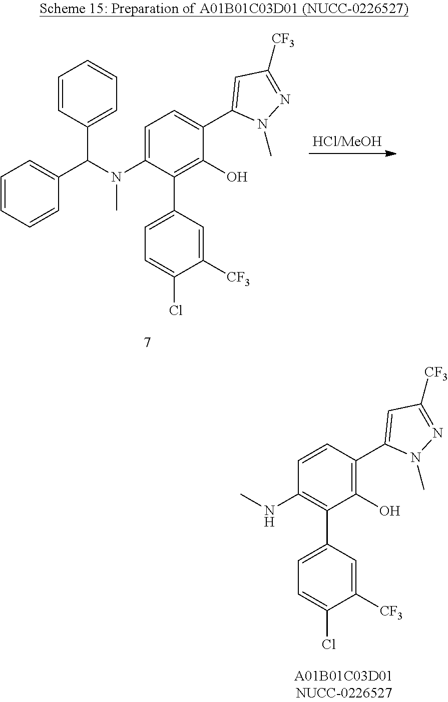 [SMILES: CN(c1ccc(-c2cc(C(F)(F)F)nn2C)c(O)c1-c1ccc(Cl)c(C(F)(F)F)c1)C(c1ccccc1)c1ccccc1 ; "HCl/MeOH" ; CNc1ccc(-c2cc(C(F)(F)F)nn2C)c(O)c1-c1ccc(Cl)c(C(F)(F)F)c1]

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min. the flow rate was 0.8 ml/min. Mobile phase A was H₂O+10 mM NH₄HCO₃, mobile phase B was Acetonitrile. The column used for chromatography was a Xbridge C18 2.1*50 mm column (Sum particles). Detection methods are diode array (DAD) and evaporative light scattering (ELSD) detection. MS mode was positive electrospray ionization. MS range was 100-1000.

5_95CD_6min-220-254-ELSD: LC/MS (The gradient was 5% B in 0.40 min and 5-95% B at 0.40-3.40 min. hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge C18 2.1*50 mm column (Sum particles). Detection methods are diode array (DAD) detection. MS mode was positive electrospray ionization. MS range was 100-1000.

### Experimentals for Largest Scale Run

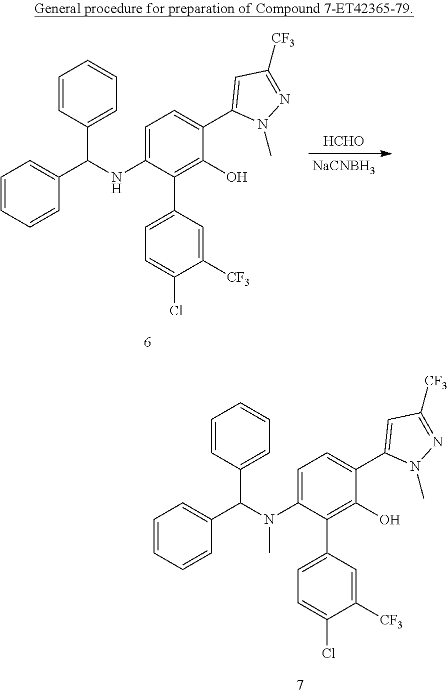

To a solution of Compound 6 (500 mg, 830.61 μmol, 1 eq) in AcOH (10 mL) was added paraformaldehyde (249.40 mg, 8.31 mmol, 10 eq). After stirring at 20° C. for 1.5 hrs, NaBH₃CN (260.99 mg, 4.15 mmol, 5 eq) was added. The mixture was stirred at 25° C. for 12 hrs. LCMS showed the starting material was consumed and a new peak with desired product Ms was detected. The reaction was concentrated to give crude product, which was diluted with water (50 mL) and extracted with ethyl acetate (3×50 mL). The organic layer was separated and the combined organic layer was washed with brine (50 mL), dried over Na₂SO₄, filtered and concentrated under reduced pressure to crude Compound 7 (750) mg, yield 58.63%) was obtained as a yellow solid. The crude product was used to the next step directly without further purification.

$$LCMS ( ESI + ) : RT = 0.969 min , m / z 616.3 {( M + H )}^{+} .$$

5-95AB 2 min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

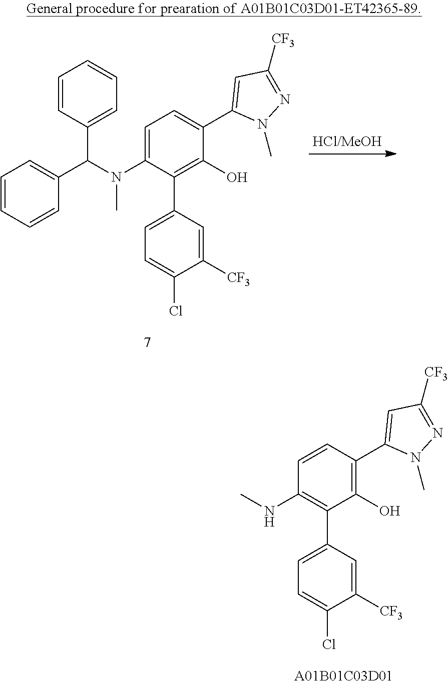 [SMILES: CN(c1ccc(-c2cc(C(F)(F)F)nn2C)c(O)c1-c1ccc(Cl)c(C(F)(F)F)c1)C(c1ccccc1)c1ccccc1 ; "HCl/MeOH" ; CNc1ccc(-c2cc(C(F)(F)F)nn2C)c(O)c1-c1ccc(Cl)c(C(F)(F)F)c1]

To a solution of Compound 7 (550 mg, 892.86 μmol, 1 eq) in MeOH (10 mL) was added con. HCl (2.64 g, 26.79 mmol, 2.59 mL, 37% purity, 30 eq). The mixture was stirred at 80° C. for 24 hrs. LCMS showed about 16% of the starting material was remaining and a new peak with desired product Ms was detected. The reaction was concentrated to give crude product, which was diluted with water (50 mL) and extracted with ethyl acetate (3×50 mL). The organic layer was separated and the combined organic layer was washed with brine (50 mL), dried over Na₂SO₄, filtered and concentrated under reduced pressure to give crude product. The crude product was purified by column chromatography on silica gel (eluted with petroleum ether/ethyl acetate=50/1 to 10/1) to give A01B01C03D01 (330 mg, yield 69.85%) as a yellow solid.

$$LCMS ( ESI + ) : RT = 0.852 min , m / z 450.2 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

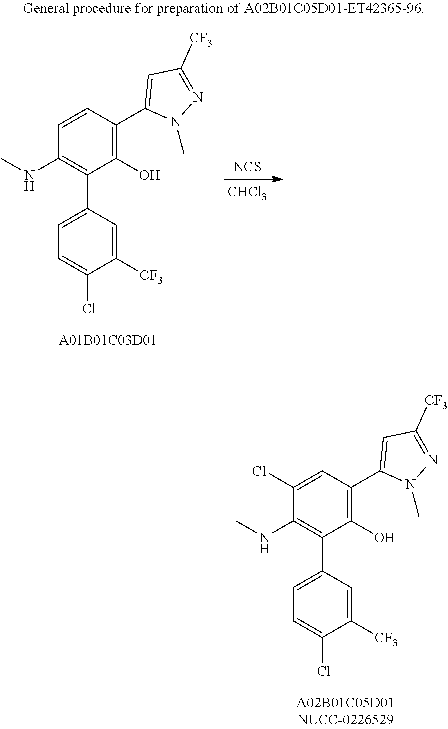

To a solution of A01B01C03D01 (300 mg, 566.95 μmol, 85% purity, 1 eq) in CHCl₃ (12 mL) was added NCS (227.12 mg, 1.70 mmol, 3 eq). The mixture was stirred at 20° C. for 12 hrs. LCMS showed about 45% of the starting material was remaining and 26% of desired product was detected. The reaction was concentrated to give crude product. The crude product was purified by Prep-HPLC (column: Waters Xbridge Prep OBD C18 150*40 mm*10 um; mobile phase [water (10 mM NH₄HCO₃)-ACN]; B %: 50%-80%, 8 min) to give A02B01C05D01 (65.4 mg, yield 23.82%) as a white solid.

¹H NMR (ET42365-96-P1A, 400 MHz, CHLOROFORM-d) δ 2.39 (s, 3H), 3.86 (s, 3H), 4.89 (s, 1H), 6.55 (s, 1H), 7.22 (s, 1H), 7.55-7.60 (m, 1H), 7.64-7.69 (m, 1H), 7.78 (d, J=1.88 Hz, 1H)

$$LCMS ( ESI + ) : RT = 2.52 min , m / z 484.1 {( M + H )}^{+} .$$

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (5 um particles). Detection methods are diode array (DAD) and evaporative light scattering (ELSD) detection. MS mode was positive electrospray ionization. MS range was 100-1000.

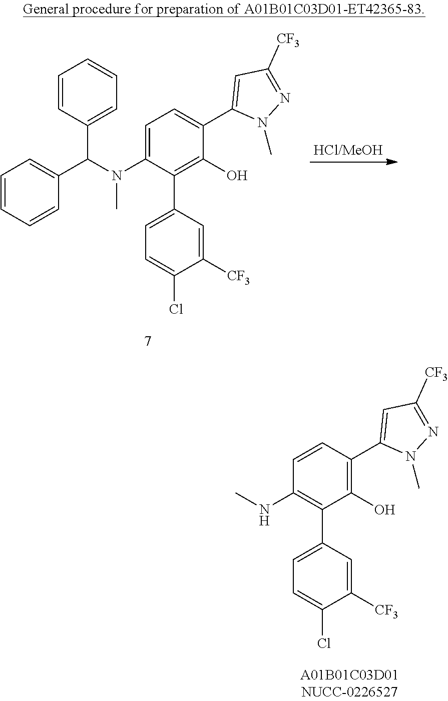 [SMILES: CN(c1ccc(-c2cc(C(F)(F)F)nn2C)c(O)c1-c1ccc(Cl)c(C(F)(F)F)c1)C(c1ccccc1)c1ccccc1 ; "HCl/MeOH" ; CNc1ccc(-c2cc(C(F)(F)F)nn2C)c(O)c1-c1ccc(Cl)c(C(F)(F)F)c1]

To a solution of Compound 7 (200 mg, 324.68 μmol, 1 eq) in MeOH (2 mL) was added HCl (959.84 mg, 9.74 mmol, 37% purity, 30 eq). The mixture was stirred at 80° C. for 12 hrs. LCMS showed the starting material was consumed and a new peak with desired product Ms was detected. The reaction was concentrated to give crude product. The crude product was purified by Prep-HPLC (column: Waters Xbridge Prep OBD C18 150*40 mm*10 um; mobile phase: [water (10 mM NH₄HCO₃)-ACN]; B %: 55%-75%, 8 min) to give A01B01C03D01 (25.7 mg, yield 17.19%) as a pink solid.

¹H NMR (ET42365-83-P1A, 400 MHz, CHLOROFORM-d) δ 2.84 (s, 3H), 3.85 (s, 3H), 4.78 (s, 1H), 6.42 (br d, J=8.63 Hz, 1H), 6.55 (s, 1H), 7.16 (d, J==8.38 Hz, 1H), 7.52 (br d, J=7.88 Hz, 1H), 7.66-7.76 (m, 2H)

$$LCMS ( ESI + ) : RT = 3.551 min , m / z 450.1 {( M + H )}^{+} .$$

5_95CD_6min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge C18 2.1*50 mm column (5 um particles). Detection methods are diode array (DAD) detection. MS mode was positive electrospray ionization. MS range was 100-1000.

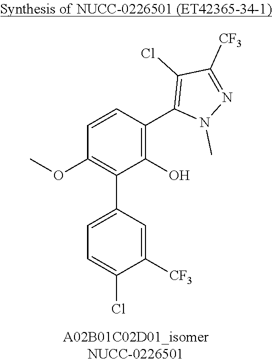 [SMILES: COc1ccc(-c2c(Cl)c(C(F)(F)F)nn2C)c(O)c1-c1ccc(Cl)c(C(F)(F)F)c1]

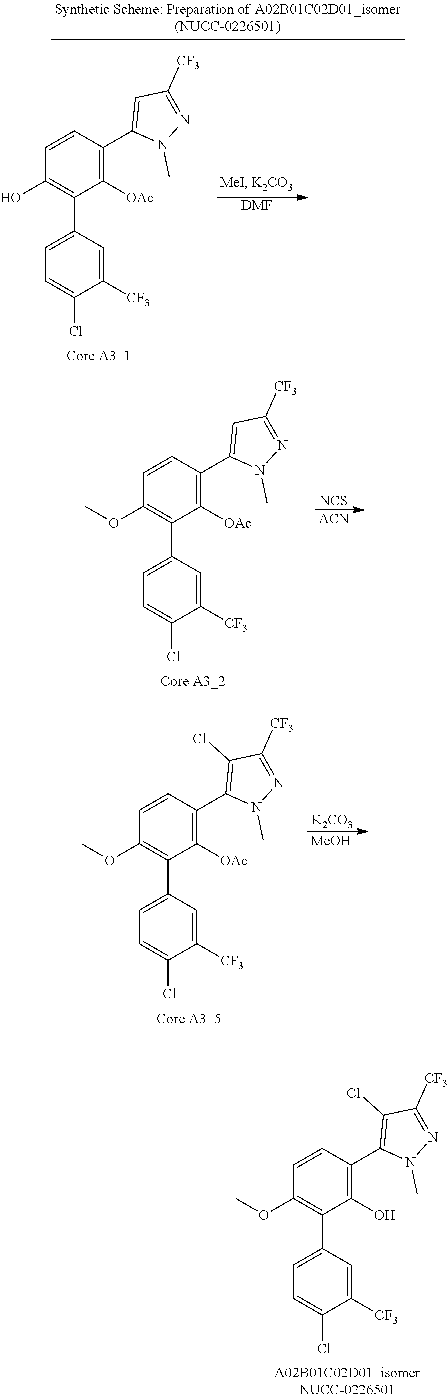

### Chemical Synthesis

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min. the flow rate was 0.8 ml/min. Mobile phase A was H₂O+10 mM NH₄HCO₃, mobile phase B was Acetonitrile. The column used for chromatography was a Xbridge C18 2.1*50 mm column (Sum particles). Detection methods are diode array (DAD) and evaporative light scattering (ELSD) detection. MS mode was positive electrospray ionization. MS range was 100-1000.

### Experimentals for Largest Scale Run

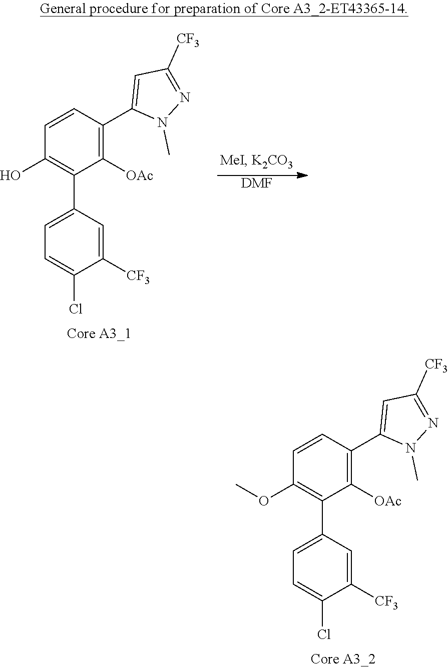

To a solution of Core A3_1 (200 mg, 417.74 μmol, 1 eq) in DMF (4 mL) were added K₂CO₃ (115.47 mg, 835.47 μmol, 2 eq) and MeI (118.59 mg, 835.47 μmol, 2 eq), the reaction was stirred at 20° C. for 12 hrs. LCMS showed the starting material was consumed and a new peak with desired product Ms was detected. The reaction was diluted with water (20 mL) and extracted with ethyl acetate (3×20 mL). The organic layer was separated and the combined organic layer was washed with brine (30 mL), dried over Na2SO4, filtered and concentrated under reduced pressure to give Core A3_2 (180 mg, yield 78.69%) as a yellow oil. The crude product was used to the next step directly without further purification.

$$LCMS ( ESI + ) : RT = 0.862 min , m / z 493.2 {( M + H )}^{+} .$$

5-95AB 2 min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

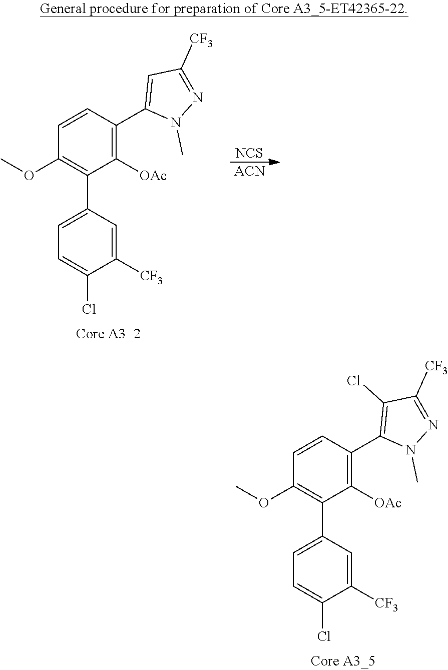

To a solution of Core A3_2 (180 mg, 365.26 μmol, 1 eq) in CH₃CN (4 mL) was added NCS (53.65 mg, 401.79 μmol, 1.1 eq) in portions at 20° C., the reaction was stirred at 80° C. for 12 hrs. LCMS showed the starting material was consumed and a new peak with desired product Ms was detected. The reaction was concentrated under high vacuum to give the crude product. The crude product was purified by Prep-TLC (petroleum ether/ethyl acetate=2/1) to give Core A3_5 (110 mg, yield 51.41%) as a white solid.

¹H NMR (ET42365-22-P1A, 400 MHz, CHLOROFORM-d) δ 1.78 (s, 3H), 3.78 (s, 3H), 3.87 (s, 3H), 7.06 (d, J=8.63 Hz, 1H), 7.37 (d, J=8.63 Hz, 1H), 7.46 (dd, J=8.25, 1.75 Hz, 1H), 7.56 (d, J=8.25 Hz, 1H), 7.67 (d, J=1.63 Hz, 1H)

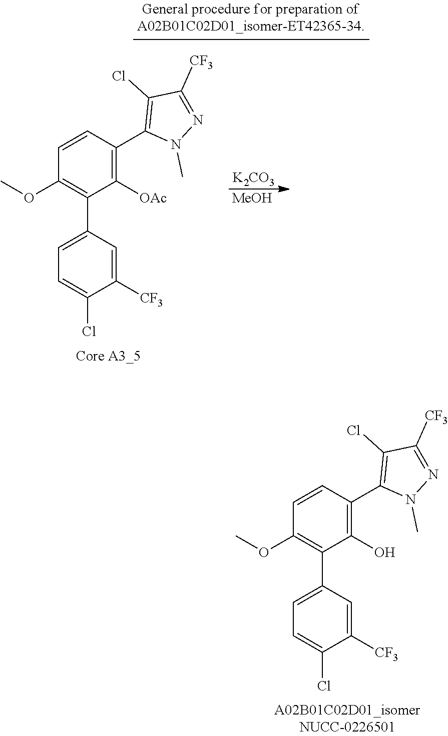

To a solution of Core A3_5 (100 mg, 189.67 μmol, 1 eq) in MeOH (5 mL) was added K₂CO₃ (52.43 mg, 379.33 μmol, 2 eq), the reaction was stirred at 20° C. for 12 hrs. LCMS showed the starting material was consumed and a new peak with desired product Ms was detected. The reaction was diluted with water (30 mL), adjust to pH=6-7 and extracted with ethyl acetate (3×15 mL). The organic layer was separated and the combined organic layer was washed with brine (20 mL), dried over N₂SO₄, filtered and concentrated under reduced pressure to crude product. The crude product was purified by Prep-HPLC (column: Waters Xbridge Prep OBD C18 150*40 mm*10 um; mobile phase: [water (10 mM NH₄HCO₃)-ACN]; B %: 65%-95%, 8 min) to give A02B01C02D01_isomer (84.5 mg, yield 90.99%) as a white solid.

¹H NMR (ET42365-34-P1A, 400 MHz, CHLOROFORM-d) δ 3.82 (d, J=4.75 Hz, 6H), 4.97 (s, 1H), 6.76 (d, J=8.76 Hz, 1H), 7.29 (d, J=8.63 Hz, 1H), 7.53 (dd, J=8.13, 2.00 Hz, 1H), 7.66 (d, J=8.25 Hz, 1H), 7.75 (d, J=1.88 Hz, 1H)

$$LCMS ( ESI + ) : RT = 2.439 min , m / z 485.1 {( M + H )}^{+} .$$

50_100CD 6 min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (5 um particles). Detection methods are diode array (DAD) and evaporative light scattering (ELSD) detection. MS mode was positive electrospray ionization. MS range was 100-1000.

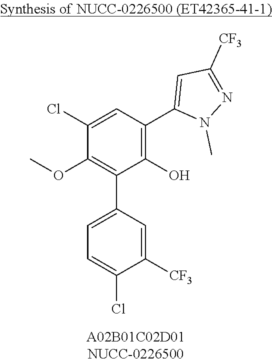 [SMILES: COc1c(Cl)cc(-c2cc(C(F)(F)F)nn2C)c(O)c1-c1ccc(Cl)c(C(F)(F)F)c1]

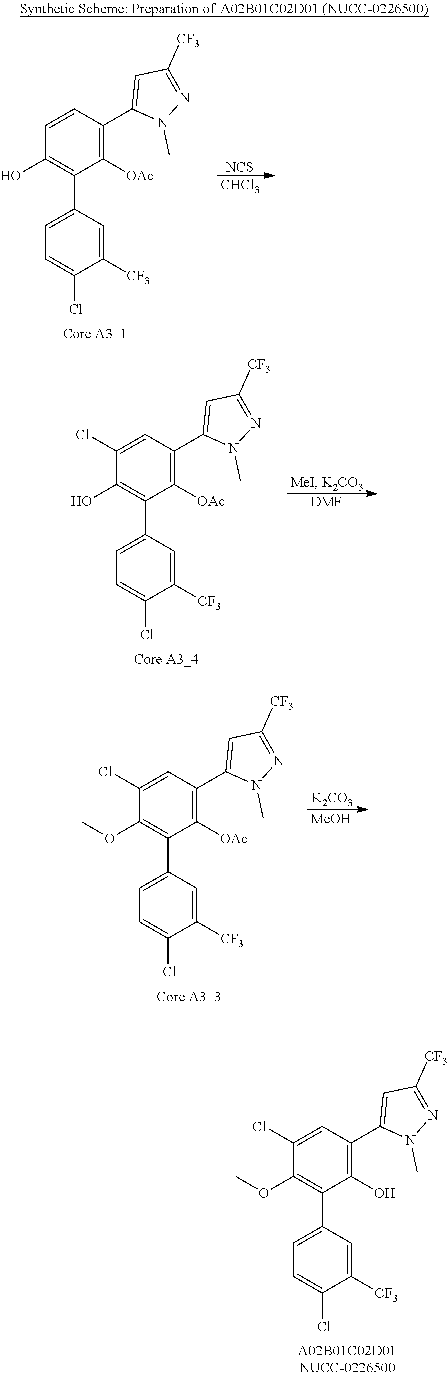

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (5 um particles). Detection methods are diode array (DAD) and evaporative light scattering (ELSD) detection. MS mode was positive electrospray ionization. MS range was 100-1000.

### Experimentals for Largest Scale Run

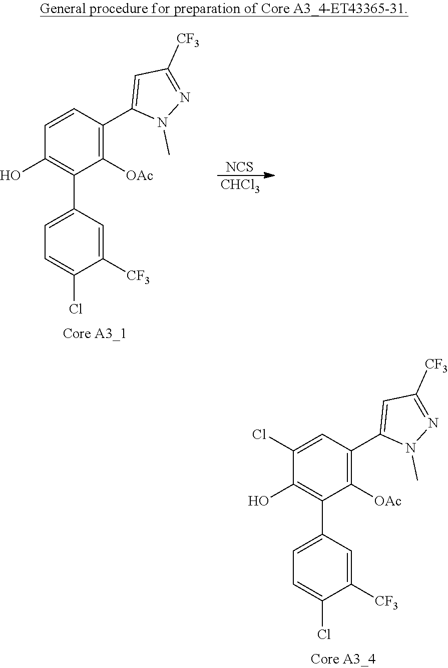

To a solution of Core A3_1 (700 mg, 1.46 mmol, 1 eq) and N-isopropylpropan-2-amine (295.89 mg, 2.92 mmol, 413.26 μL, 2 eq) in CHCl₃ (25 mL) was added NCS (214.76 mg, 1.61 mmol, 1.1 eq) at 0° C., the reaction was stirred at 25° C. for 24 hrs. LCMS showed about 28% of the starting material was remaining and 64% of desired product was detected. The reaction was concentrated under high vacuum to give the crude product. The crude product was purified by column chromatography on silica gel (eluted with petroleum ether/ethyl acetate=50/1 to 10/1) to give Core A3_4 (200 mg, yield 25.32%) as a white solid.

¹H NMR (ET42365-31-P1A, 400 MHz, CHLOROFORM-d) δ 1.78 (s, 3H), 3.83 (s, 3H), 5.92 (s, 1H), 6.51 (s, 1H), 7.42 (s, 1H), 7.50 (dd, J=8.25, 1.50 Hz, 1H), 7.61 (d, J=8.38 Hz, 1H), 7.72 (d, J=1.38 Hz, 1H)

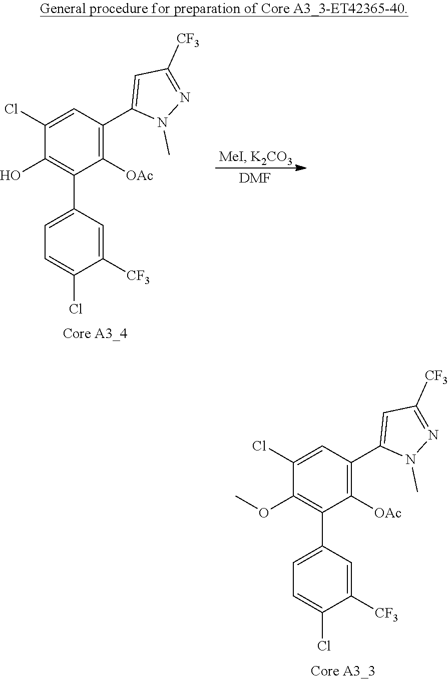

To a solution of Core A3_4 (200 mg, 389.70 μmol, 1 eq) and K₂CO₃ (107.72 mg, 779.40 μmol, 2 eq) in DMF (5 mL) was added MeI (110.63 mg, 779.40 μmol, 2 eq), the reaction was stirred at 25° C. for 2 hrs. LCMS showed the starting material was consumed and a new peak with desired product Ms was detected. The reaction was diluted with water (20 mL) and extracted with ethyl acetate (3×15 mL). The organic layer was separated and the combined organic layer was washed with brine (20 mL), dried over Na₂SO₄, filtered and concentrated under reduced pressure to give Core A3_3 (150 mg, yield 69.35%) as a yellow oil. The crude product was used to the next step directly without further purification.

$$LCMS ( ESI + ) : RT = 0.892 min , m / z 527.2 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

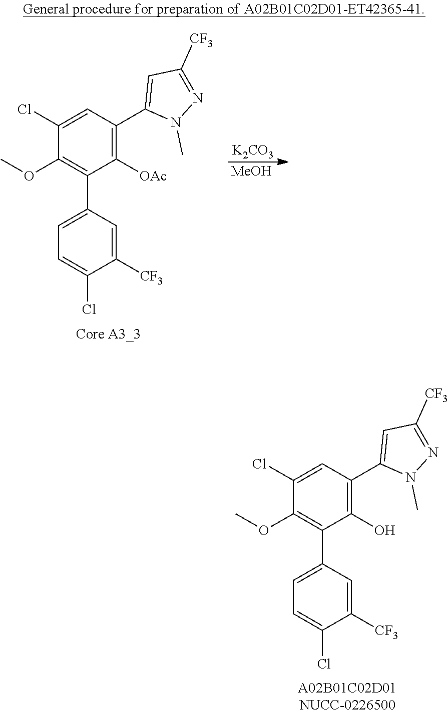

To a solution of Core A3_3 (150 mg, 284.50 μmol, 1 eq) in MeOH (5 mL) was added K₂CO₃ (78.64 mg, 569.00 μmol, 2 eq), the reaction was stirred at 20° C. for 12 hrs. LCMS showed the starting material was consumed and a new peak with desired product Ms was detected. The reaction was diluted with water (30 mL), adjust to pH=6-7 and extracted with ethyl acetate (3×15 mL). The organic layer was separated and the combined organic layer was washed with brine (20 mL), dried over Na₂SO₄, filtered and concentrated under reduced pressure to crude product. The crude product was purified by Prep-HPLC (column: Waters Xbridge Prep OBD C18 150*40 mm*10 um; mobile phase: [water (10 mM NH₄HCO₃)-ACN]; B %: 65%-95%, 8 min) to give A02B01C02D01 (106.5 mg, yield 75.69%) as off-white solid.

¹H NMR (ET42365-41-P1A, 400 MHz, CHLOROFORM-d) δ 3.59 (s, 3H), 3.87 (s, 3H), 5.06 (s, 1H), 6.61 (s, 1H), 7.34 (s, 1H), 7.56-7.62 (m, 1H), 7.65-7.71 (m, 1H), 7.80 (s, 1H)

$$LCMS ( ESI + ) : RT = 2.501 min , m / z 485.1 {( M + H )}^{+} .$$

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (5 um particles). Detection methods are diode array (DAD) and evaporative light scattering (ELSD) detection. MS mode was positive electrospray ionization. MS range was 100-1000.

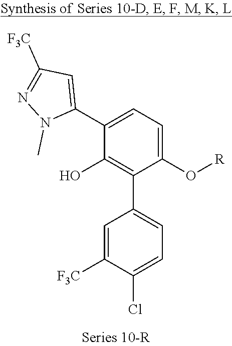

Scheme 16: The route to synthesize Series 10-D, Series 10-E, Series 10-F, Series 10-M, Series 10-K-P1, Series 10-K-P2, Series 10-L-P1 and Series 10-L-P2. We take Series 10-E as an example.

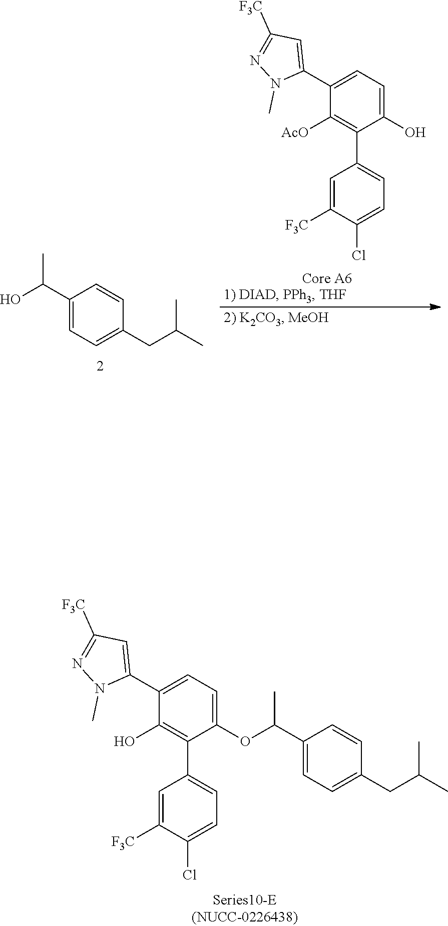

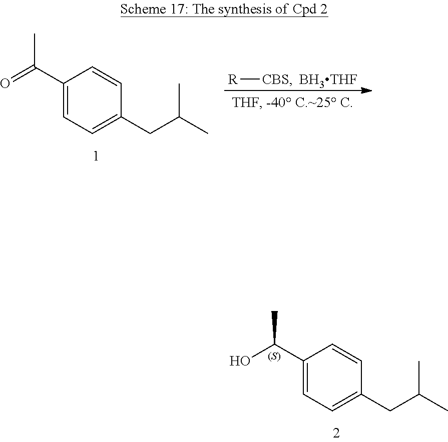

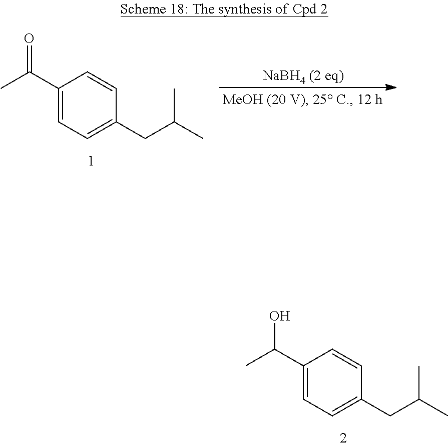

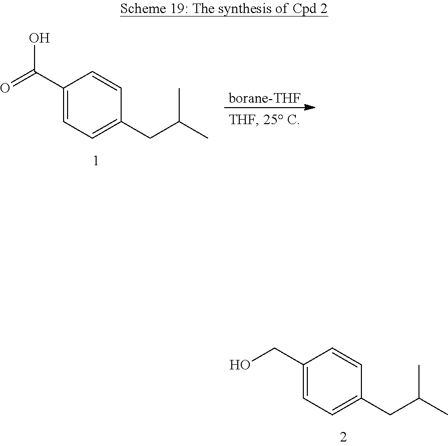

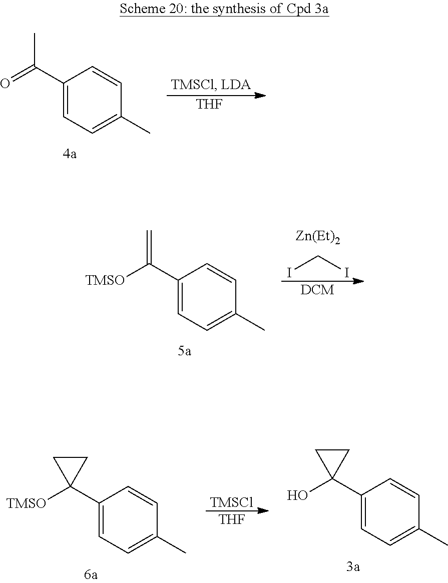

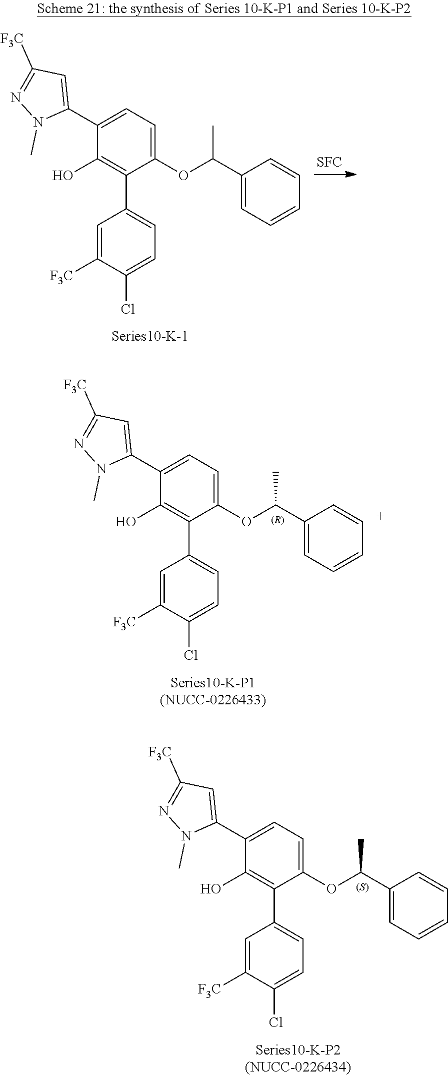

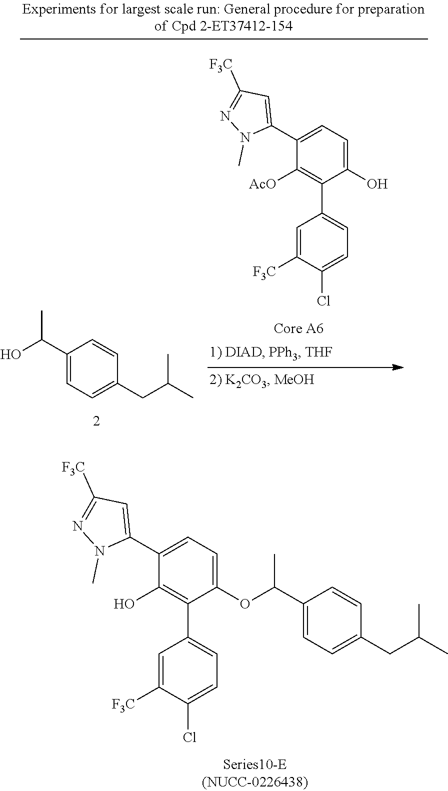 [SMILES: CC(=O)Oc1c(-c2cc(C(F)(F)F)nn2C)ccc(O)c1-c1ccc(Cl)c(C(F)(F)F)c1 ; CC(C)Cc1ccc(C(C)O)cc1 ; CC(C)Cc1ccc(C(C)Oc2ccc(-c3cc(C(F)(F)F)nn3C)c(O)c2-c2ccc(Cl)c(C(F)(F)F)c2)cc1]

A mixture of Core A6 (100 mg, 208.87 μmol), Cpd 2 (55.85 mg, 313.30 μmol), diisopropyl azodicarboxylate (84.47 mg, 417.74 μmol, 81.22 μL), triphenylphosphine (109.57 mg, 417.74 μmol) in tetrahydrofuran (2 mL) was degassed and purged with N₂ for 3 times, and then the reaction was stirred at 25° C. for 12 h. After that K₂CO₃ (57.73 mg, 417.74 mol) was added to the reaction, the mixture was stirred at 25° C. for 1 h. LCMS showed the reaction was completed. The reaction was poured into water (2 mL) and extracted with ethyl acetate (5 mL). The organic layer was concentrated to give a residue which was purified by Prep-HPLC (column: Waters Xbridge BEH C18 100*30 mm*10 um; mobile phase: [water (10 mM NH₄HCO₃)-acetonitrile]; B %: 85%-98%, 10 min) to give Series 10-E (9 mg, yield 7%) as white solid.

¹H NMR: ET37412-154-HNMRP1 (400 MHz, CDCl₃) δ 0.90 (d, J=6.6 Hz, 6H), 1.50 (br d, J=6.3 Hz, 3H), 1.85 (quind, J=6.7, 13.4 Hz, 1H), 2.46 (d, J=7.2 Hz, 2H), 5.08 (s, 1H), 3.81 (s, 3H), 5.32 (q, J=6.3 Hz, 1H), 6.53 (s, 1H), 6.60 (d, J=8.7 Hz, 1H), 7.15-7.05 (m, 5H), 7.60-7.53 (m, 1H), 7.67-7.62 (m, 1H), 7.83 (s, 1H)

### LCMS Method:

$$LCMS ( ESI + ) : m / z = 579.3 {( M + H )}^{+} , RT : 3.534 min$$

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (Sum particles). Detection methods are diode array (DAD) and evaporative light scattering (ELSD) detection as well as positive electrospray ionization. MS range was 100-1000.

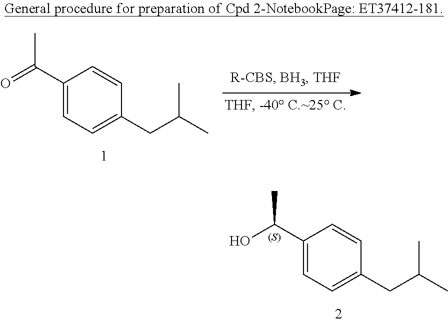

To a solution of Cpd 1 (1 g, 5.67 mmol) in tetrahydrofuran (10 mL) was added borane tetrahydrofuran complex (6.81 mL, 6.81 mmol) at −40° C. To the mixture reaction was added chloro-bis[(1R,2S,3R,5R)-2,6,6-trimethylnorpinan-3-yl]borane (2.18 g, 6.81 mmol) in tetrahydrofuran (5 mL) at −40° C. and the reaction was stirred at 25° C. for 2 h. The reaction was poured into water (20 mL). The organic phase was separated and the aqueous phase was extracted with ethyl acetate (10 mL) for three times. The organic phase was washed with brine (50 mL), dried over Na₂SO₄, filtered and concentrated under vacuum to give crude product. The crude product was purified by column chromatography on silica gel (petroleum ether/ethyl acetate=1/0 to 1/2) to give Cpd 2 (0.5 g. yield 45%) as colorless oil.

¹H NMR (15017259-181-P1 400 MHz, CHLOROFORM-d)

δ 0.92 (d, J=6.62 Hz, 6H), 1.50 (d, J=6.62 Hz, 3H), 1.83-1.92 (m, 2H), 2.48 (d, J=7.28 Hz, 2H), 4.88 (q, J=6.39 Hz, 1H), 7.14 (d, J=7.94 Hz, 2H), 7.29 (d, J=7.94 Hz, 2H)

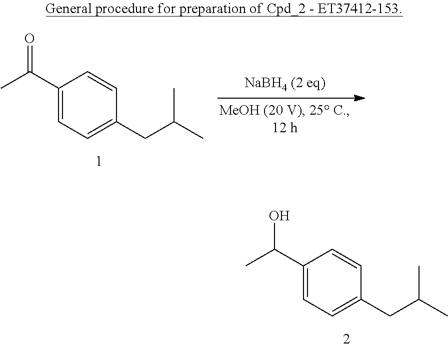

To a solution of Cpd 1 (0.5 g, 2.84 mmol) in methanol (10 mL) was added NaBH₄ (214.63 mg, 5.67 mmol). The reaction mixture was stirred at 25° C. for 12 h under N₂ atmosphere. The reaction was poured into water (20 mL) and extracted with ethyl acetate (15 mL). The organic layer was concentrated to give Cpd_2 (300 mg, 59% yield) as colorless oil.

¹H NMR (ET37412-153-HNMRP1, 400 MHz, DMSO-d₆)

δ 0.85 (d, J=6.63 Hz, 6H), 1.29 (d, J=6.38 Hz, 3H), 1.73-1.85 (m, 1H), 2.41 (d, J=7.13 Hz, 2H), 7.07 (d, J=7.88 Hz, 2H), 7.23 (d, J=7.88 Hz, 2H)

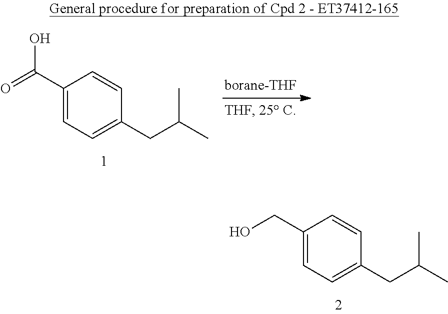

To a solution of Cpd 1 (0.5 g, 2.81 mmol) in tetrahydrofuran (5 mL) was added BH₃·THF (1 M, 5.61 mL). The mixture was stirred at 25° C. for 12 hr. TLC showed the reaction was completed. The reaction was quenched with methanol (5 mL) and then the mixture was concentrated to give a residue which was purified by Prep-TLC to give Cpd 2 (0.3 g, yield 65%) as colorless oil.

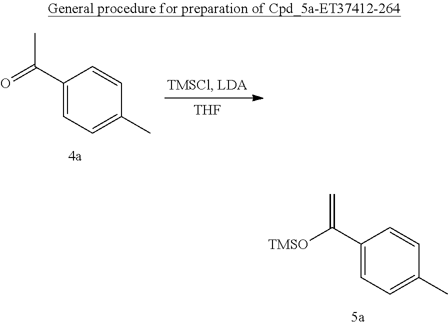

To a solution of Cpd 4a (5 g, 37.26 mmol) in tetrahydrofuran (50 mL) at 0° C. was added lithium diisopropylamide (2 M, 20.50 mL) and chlorotrimethylsilane (4.45 g, 40.99 mmol). The reaction was stirred at 25° C. for 12 h. TLC showed the reaction was completed. The reaction was poured into water (50 mL) and extracted with ethyl acetate (60 mL). The organic layer was dried and concentrated to give Cpd 5a (5 g, yield 65%) as colorless oil which was used to next step directly.

¹H NMR (ET37412-264-1, 400 MHz, CDCl₃-d₆)

δ 0.08-0.14 (m, 9H), 2.19 (s, 3H), 4.23 (d, J=1.47 Hz, 1H), 4.61-4.82 (m, 1H), 4.71 (d, J=1.59 Hz, 1H), 6.97 (d, J=8.07 Hz, 2H), 7.33 (d, J=8.19 Hz, 2H)

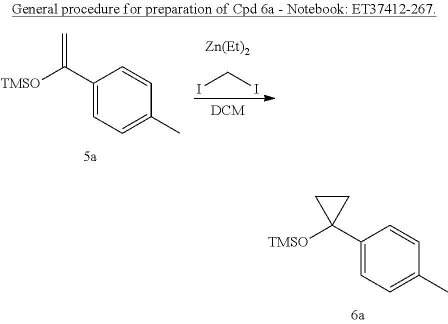

To a solution of Cpd 5a (4 g, 19.38 mmol) in dichloromethane (10 mL) at 0° C. was added CH₂I₂ (7.79 g, 29.08 mmol) and diethylzinec (1 M, 29.08 mL). The reaction mixture was stirred at 25° C. for 12 h. TLC showed the reaction was completed. The reaction was poured into water (10 mL) and extracted with ethyl acetate (20 mL). The organic layer was dried and concentrated to give Cpd 6a (1 g, yield 23%) as yellow oil which was used to next step directly.

¹H NMR (ET37412-267-11, 400 MHz, CDCl₃-d₆) δ 0.00 (s, 9H), 0.85-0.95 (m, 2H), 1.06-1.16 (m, 2H), 2.26 (s, 3H), 7.15-7.23 (m, 4H)

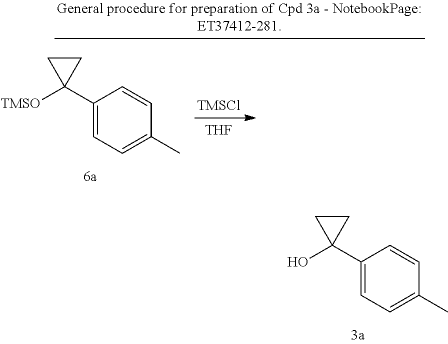

To a solution of Cpd 6a (1 g, 12.71 mmol) in tetrahydrofuran (10 mL) was added chlorotrimethylsilane (0.41 g, 15.25 mmol) at 0° C. The reaction mixture was stirred at 25° C. for 1 h. TLC showed the reaction was completed. The reaction was poured into water (10 mL) and extracted with ethyl acetate (20 mL). The organic layer was dried and concentrated to give Cpd 3a (1 g, yield 53%) as yellow oil which was used to next step directly.

¹H NMR (ET37412-281-2, 400 MHz, CDCl₃-d₆)

δ 0.95-1.05 (m, 2H), 1.18-1.25 (m, 2H), 2.34 (s, 3H), 7.11-7.25 (m, 4H)

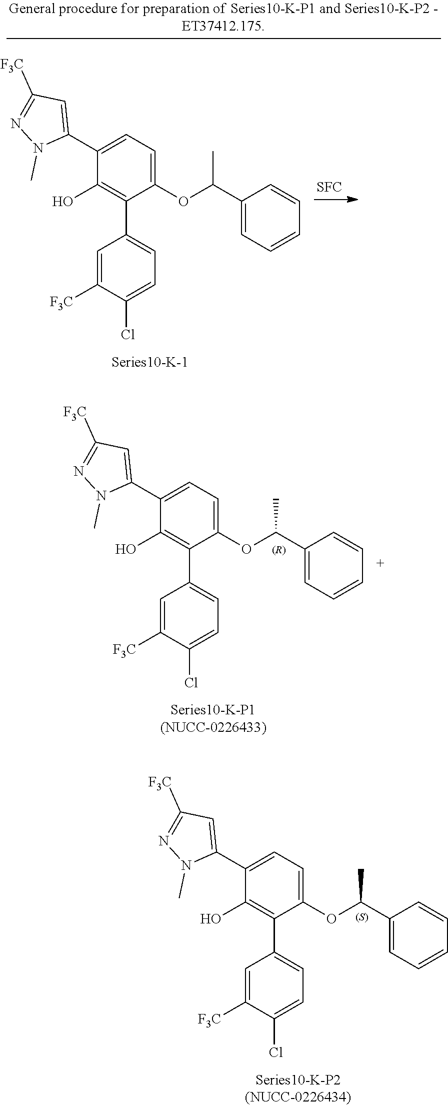

40 mg of Series10-K-1 was separated by SFC (Column: DAICEL CHIRALCEL OJ (250 mm×30 mm, 10 um); mobile phase: [0.1% NH₃H₂O MEOH]; B %: 50%-50%, 15 min Column: Chiralcel OJ-3, 50×4.6 mm I.D., 3 um, Mobile phase: A: CO₂ B: MeOH (0.05% IPAm, v/v), Flow rate: 3.4 mL/min, Column temp.: 35° C., ABPR: 1800 psi). Two fractions was concentrated to give Series10-K-P1 (24 mg, yield 64%) and Series10-K-P2 (12 mg, yield 33%).

¹H NMR (ET37412-175-1, 400 MHz, MeOD)

δ 1.45 (d, J=6.17 Hz, 3H), 3.76 (s, 3H), 5.43 (d, J=6.39 Hz, 1H), 6.54 (s, 1H), 6.50-6.58 (m, 1H), 6.66 (d, J=8.60 Hz, 1H), 7.10 (d, J=8.60 Hz, 1H), 7.18-7.33 (m, 5H), 7.59-7.65 (m, 1H), 7.66-7.72 (m, 1H), 7.78 (s, 1H)

$$LCMS ( ESI + ) : m / z = 541.1 {( M + H )}^{+} , RT : 3.187 min$$

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

SFC Method:
Column: Chiralcel OJ-3, 50 × 4.6 mm I.D., 3 um
Mobile phase: A: CO₂ B: MeOH (0.05% IPAm, v/v)
Gradient:
Time A % B %
0.0 95 5
0.2 95 5
1.2 50 50
2.2 50 50
2.6 95 5
3.0 95 5
Flow rate: 3.4 mL/min
Column temp.: 35° C.
ABPR: 1800 psi

¹H NMR (ET37412-175-2, 400 MHz, MeOD) δ 1.45 (d, J=6.17 Hz, 3H), 3.76 (s, 3H), 5.43 (q, J=6.84 Hz, 1H), 6.54 (s, 1H), 6.65 (d, J=8.82 Hz, 1H), 7.10 (d, J=8.60 Hz, 1H), 7.17-7.36 (m, 5H), 7.60-7.66 (m, 1H), 7.67-7.71 (m, 1H), 7.78 (s, 1H)

$$LCMS ( ESI + ) \text{:} m / z = 541.1 {( M + H )}^{+} , RT \text{:} 3.187 min$$

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (Sum particles). Detection methods are diode array (DAD) and evaporative light scattering (ELSD) detection as well as positive electrospray ionization. MS range was 100-1000.

SFC Method:
Column: Chiralcel OJ-3, 50 × 4.6 mm I.D., 3 um
Mobile phase: A: CO₂ B: MeOH (0.05% IPAm, v/v)
Gradient:
Time A % B %
0.0 95 5
0.2 95 5
1.2 50 50
2.2 50 50
2.6 95 5
3.0 95 5
Flow rate: 3.4 mL/min
Column temp.: 35° C.
ABPR: 1800 psi

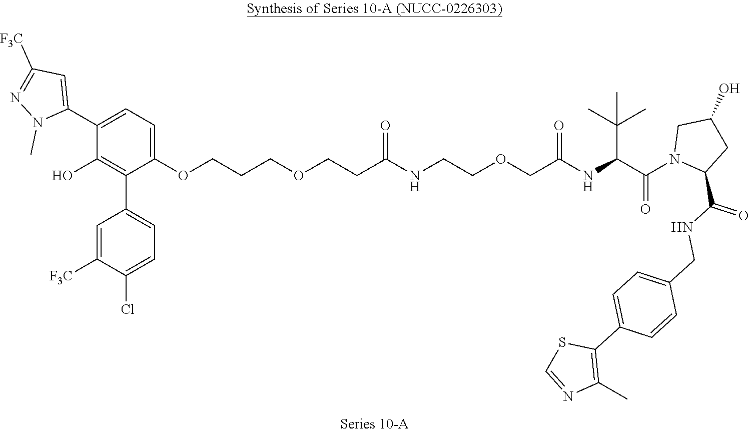 [SMILES: Cc1ncsc1-c1ccc(CNC(=O)[C@@H]2C[C@@H](O)CN2C(=O)[C@@H](NC(=O)COCCNC(=O)CCOCCCOc2ccc(-c3cc(C(F)(F)F)nn3C)c(O)c2-c2ccc(Cl)c(C(F)(F)F)c2)C(C)(C)C)cc1]

### Synthetic Scheme

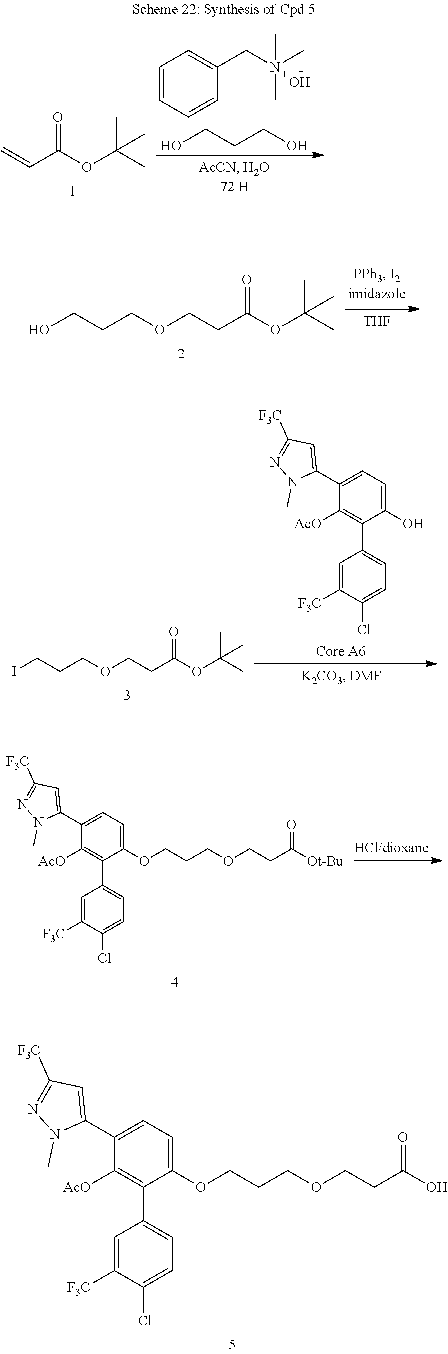

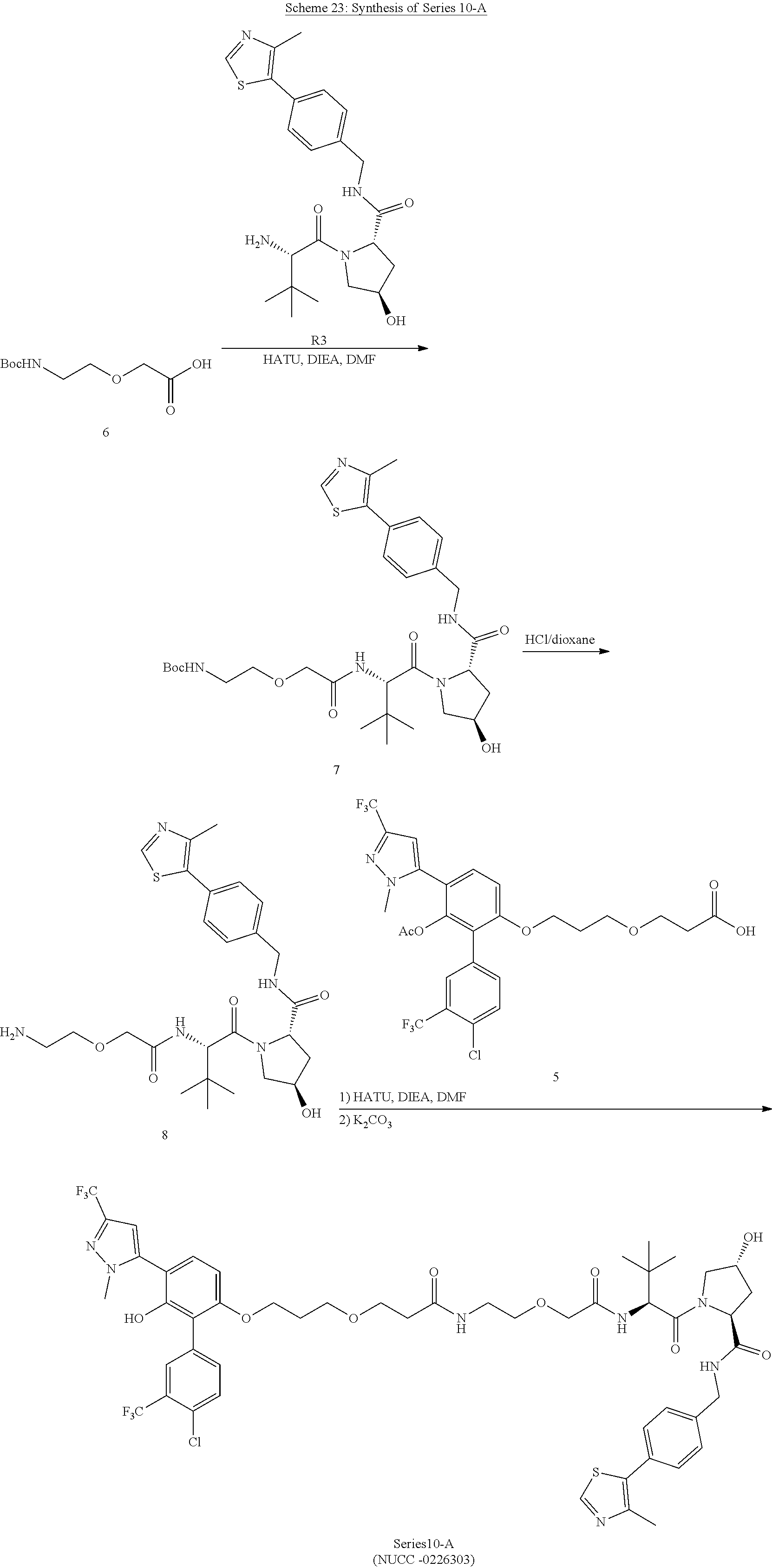

### Experiments for Largest Scale Run

### General Procedure for Preparation of Cpd 2-ET37412-122

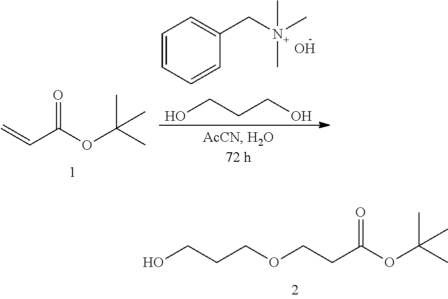

To a solution of Cpd 1 (10 g, 78.02 mmol) and propane-1,3-diol (29.69 g, 390.11 mmol) in acetonitrile (100 mL) was added benzyl(trimethyl)ammonium;hydroxide (1.21 g, 2.89 mmol). The reaction mixture was stirred for 72 h at 25° C. TLC (petroleum ether/ethyl acetate=3:1, Rf=0.5) showed the reaction was completed. The solvent was removed under reduced pressure. The residue was purified by silica gel chromatography (column height: 250 mm, diameter: 100 mm, 100-200 mesh silica gel, petroleum ether/ethyl acetate=1/0, 0/1) to give Cpd 2 (11 g, 69% yield) as colorless oil.

### General Procedure for Preparation of Cpd 3-ET37412-126

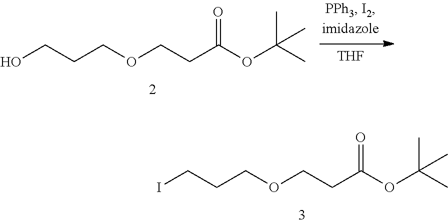

I₂ (5.59 g, 22.03 mmol), triphenylphosphine (4.62 g, 17.62 mmol) and imidazole (1.20 g, 17.62 mmol) were dissolved in tetrahydrofuran (30 mL) under inert atmospheric conditions (Argon). A solution of Cpd 2 (3 g, 14.69 mmol) in tetrahydrofuran (30 mL) was added to the reaction and the reaction was stirred at 25° C. for 12 h. LCMS showed the reaction was completed. The reaction mixture was filtered to remove the white precipitate and the solvent evaporated. The crude product was purified by silica gel chromatography eluted with petroleum ether/ethyl acetate=1/0, 0/1 to give Cpd 3 (2.4 g, 52% yield) as brown oil.

¹H NMR: ET37412-126-2 (400 MHz, CDCl₃)

δ 1.45 (s, 9H), 2.00-2.06 (m, 2H), 2.47 (t, 2H), 3.29 (br t, J=6.17 Hz, 2H), 3.49 (t, 2H), 3.65 (t, 2H)

### General Procedure for Preparation of Cpd_4-ET37412-107

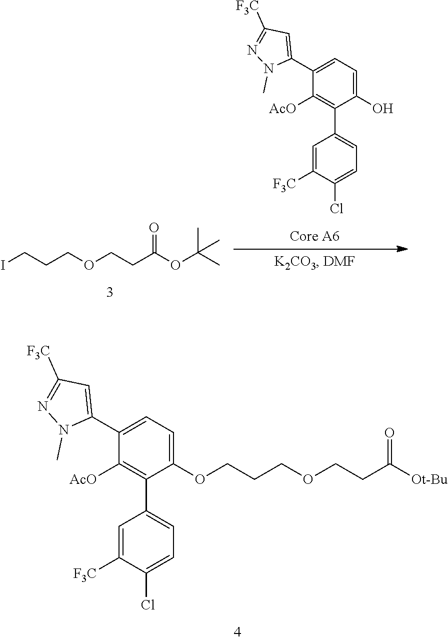 [SMILES: CC(=O)Oc1c(-c2cc(C(F)(F)F)nn2C)ccc(O)c1-c1ccc(Cl)c(C(F)(F)F)c1 ; CC(C)(C)OC(=O)CCOCCCI ; CC(=O)Oc1c(-c2cc(C(F)(F)F)nn2C)ccc(OCCCOCCC(=O)OC(C)(C)C)c1-c1ccc(Cl)c(C(F)(F)F)c1]

A mixture of Core A6 (500 mg, 1.04 mmol) and Cpd 3 (394 mg, 1.25 mmol) in N,N-dimethylformamide (I mL) was added K₂CO₃ (173 mg, 1.25 mmol). The reaction was stirred at 25° C. for 12 h. LCMS showed the reaction was completed. The reaction was poured into water (20 mL) and the reaction was extracted with ethyl acetate (20 mL). The organic layer was concentrated to give a residue which was purified by silica gel chromatography (column height: 250 mm, diameter: 100 mm, 100-200 mesh silica gel, petroleum ether/ethyl acetate=1/0, 0/1) to give Cpd 4 (400 mg, 58% yield) as colorless oil.

### LCMS Method:

$$LCMS ( ESI + ) : m / z = 665.3 ( M + H ) + , RT : 0.996 min .$$

5-95AB_2_min: LC/MS (The column used for chromatography was a Kinetex Sum EVO C18 100A. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 2.20 min 0.5% B in 0.01 min, 5-95% B (0.01-1.00 min), 95-100% B (1.00-1.80 min), 5% B in 1.81 min with a hold at 5% B for 0.39 min. The flow rate was 1.0 mL/min (0.01-1.80) 1.2 mL (1.81-2.20).

### General Procedure for Preparation of Cpd 5-ET37412-140

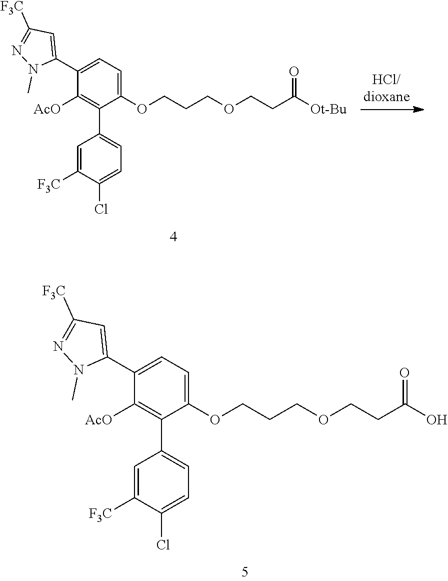 [SMILES: CC(=O)Oc1c(-c2cc(C(F)(F)F)nn2C)ccc(OCCCOCCC(=O)OC(C)(C)C)c1-c1ccc(Cl)c(C(F)(F)F)c1 ; CC(=O)Oc1c(-c2cc(C(F)(F)F)nn2C)ccc(OCCCOCCC(=O)O)c1-c1ccc(Cl)c(C(F)(F)F)c1]

A solution of Cpd 4 (380 mg, 395.71 mmol) in HCl/dioxane (5 mL, 5N) was stirred at 25° C. for 12 h. TLC (petroleum ether/ethyl acetate=1:1, Rf=0.05) showed the reaction was completed. The reaction was concentrated to give Cpd 5 (250 mg, 70% yield) as colorless oil which was used to next step without any purification.

### General Procedure for Preparation of Cpd 7-ET37412-141

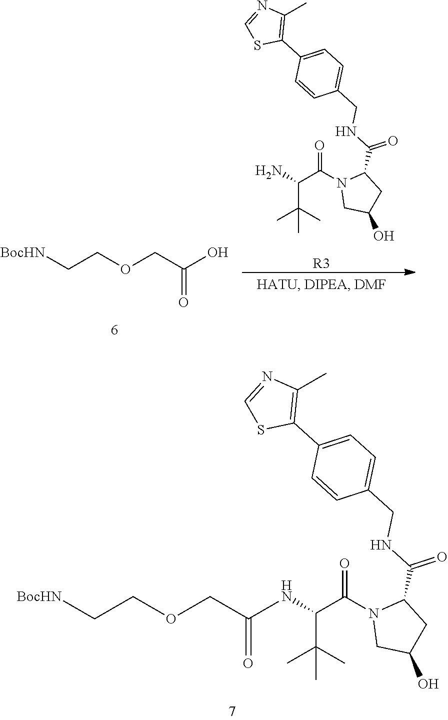

To a solution of Cpd 6 (122.20 mg, 557.41 mmol) in N,N-dimethylformamide (2 mL) was added 0-(7-Azabenzotriazol-1-yl)-N,N,N′,N′-tetramethyluronium hexafluorophosphate (264 mg, 697 mmol) and N-ethyl-N-isopropylpropan-2-amine (180.10 mg, 1.39 mmol) and R³ (200 mg, 464.51 mmol). The reaction was stirred at 25° C. for 12 h. TLC (petroleum ether/ethyl acetate=1:1, Rf=0.49) showed the reaction was completed. The reaction was poured into water (20 mL) and the reaction was extracted with ethyl acetate (20 mL). The organic layer was concentrated to give a residue which was purified by silica gel chromatography (column height: 250 mm, diameter: 100 mm, 100-200 mesh silica gel, petroleum ether/ethyl acetate=1/0, 0/1) to give Cpd 7 (250 mg, 85% yield) as yellow solid.

### General Procedure for Preparation of Cpd 8-ET37412-143

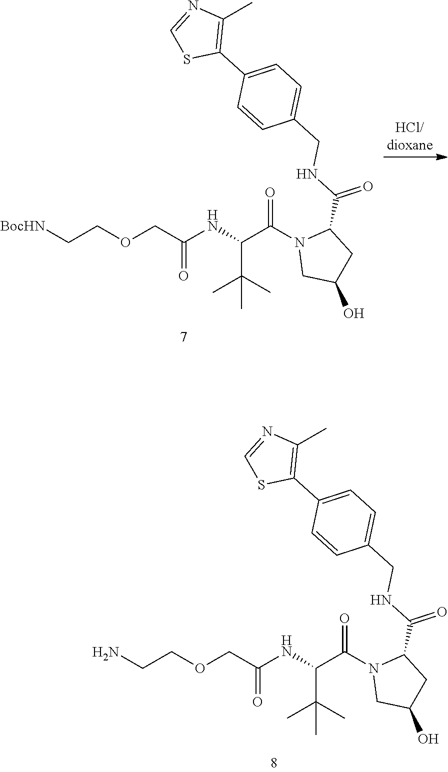 [SMILES: Cc1ncsc1-c1ccc(CNC(=O)[C@@H]2C[C@@H](O)CN2C(=O)[C@@H](NC(=O)COCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)COCCN)C(C)(C)C)cc1]

A solution of Cpd 7 (250 mg, 395.71 μmol) in HCl/dioxane (5 mL, 4 N) was stirred at 25° C. for 12 h. LCMS showed the reaction was completed. The reaction was concentrated to give Cpd 8 (250 mg, 83.18% yield) as colorless oil which was used to next step without any purification.

$$LCMS ( ESI + ) : m / z = 532.2 ( M + H ) + , RT : 0.637 min .$$

5-95AB_2_min: LC/MS (The column used for chromatography was a Kinetex 5 μm EVO C18 100A. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 2.20 min 0.5% B in 0.01 min, 5-95% B (0.01-1.00 min), 95-100% B (1.00-1.80 min), 5% B in 1.81 min with a hold at 5% B for 0.39 min. The flow rate was 1.0 mL/min (0.01-1.80) 1.2 mL (1.81-2.20).

### General Procedure for Preparation of Series 10-A-ET37412-147

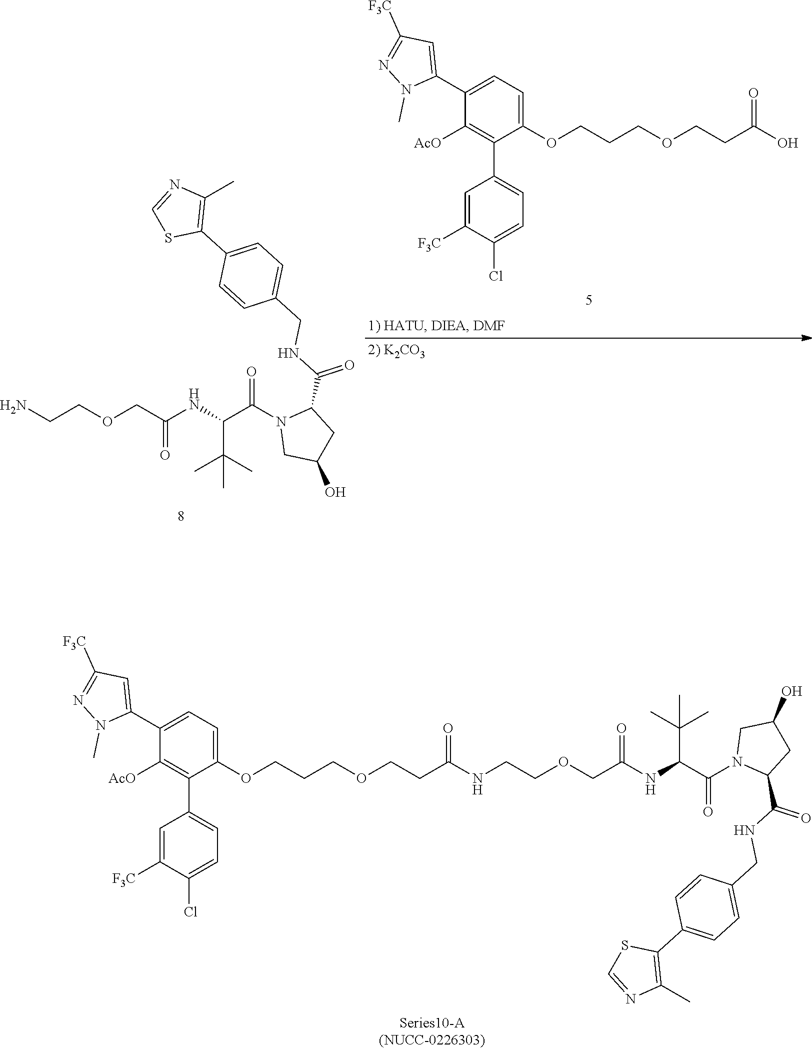 [SMILES: Cc1ncsc1-c1ccc(CNC(=O)[C@@H]2C[C@@H](O)CN2C(=O)[C@@H](NC(=O)COCCN)C(C)(C)C)cc1 ; CC(=O)Oc1c(-c2cc(C(F)(F)F)nn2C)ccc(OCCCOCCC(=O)O)c1-c1ccc(Cl)c(C(F)(F)F)c1 ; CC(=O)Oc1c(-c2cc(C(F)(F)F)nn2C)ccc(OCCCOCCC(=O)NCCOCC(=O)N[C@H](C(=O)N2C[C@@H](O)C[C@H]2C(=O)NCc2ccc(-c3scnc3C)cc2)C(C)(C)C)c1-c1ccc(Cl)c(C(F)(F)F)c1]

To a mixture of Cpd 5 (100 mg, 164 mmol) and Cpd 8 (174.6 mg, 328 mmol) in N,N-dimethylformamide (1 mL) was added N-ethyl-N-isopropylpropan-2-amine (106 mg, 821 mmol) and o-(7-Azabenzotriazol-1-yl)-N,N,N′,N′-tetramethyluronium hexafluorophosphate (124 mg, 328 mmol). The mixture was stirred at 25° C. for 12 h. After that K₂CO₃ (45.39 mg, 328.45 μmol) was added to the mixture, and the reaction was stirred at 25° C. for 1 h. LCMS showed the reaction was completed. The reaction was filtered to give a residue which was purified by Prep-HPLC (Column: Phenomenex luna C18 100*40 mm*5 um; mobile phase: [water (0.1% 2, 2,2-trifluoroacetic acid)-acetonitrile]; B %: 40%-74%, 8 min) to give Series 10-A (5 mg, 3% yield) as white solid.

¹H NMR: ET37412-147-P1A1 (400 MHz, DMSO-d₆)

δ 0.88-0.97 (m, 9H), 1.74 (br t, J=6.05 Hz, 2H), 1.90 (ddd, J=12.81, 8.71, 4.52 Hz, 1H), 2.00-2.10 (m, 1H), 2.29 (br t, J=6.36 Hz, 2H), 2.41-2.46 (m, 3H), 3.15-3.24 (m, 2H), 3.29 (br t, J=6.17 Hz, 2H), 3.43-3.52 (m, 4H), 3.56-3.67 (m, 4H), 3.73-3.74 (m, 3H), 3.94 (br d, J=1.47 Hz, 2H), 3.99 (br t, J=5.99 Hz, 3H), 4.19-4.28 (m, 1H), 4.37 (br d, J=6.60 Hz, 1H), 4.40-4.47 (m, 1H), 4.55 (d, J=9.66 Hz, 1H), 6.71 (s, 1H), 6.76 (d, J=8.68 Hz, 1H), 7.25 (d, J=8.56 Hz, 1H), 7.39 (s, 4H), 7.44 (br d, J=9.41 Hz, 1H), 7.64 (br d, J=8.31 Hz, 1H), 7.75 (dd, J=4.95, 3.24 Hz, 2H), 7.96 (br t, J=5.50 Hz, 1H), 8.57 (t, J=5.93 Hz, 1H), 8.97 (s, 1H)

$$LCMS ( ESI + ) : m / z = 1080.3 ( M + H ) + , RT : 2.723 min .$$

5_95AB_6min_MS1500-220-254-ELSD: LC/MS (The gradient was 5% B in 0.40 min and 5-95% B at 0.40-3.00 min. hold on 95% B for 1.00 min, and then 95-5% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Kinetex C18 50*2.1 mm column (Sum particles). Detection methods are diode array (DAD) and evaporative light scattering (ELSD) detection as well as positive electrospray ionization. MS range was 100-1500.

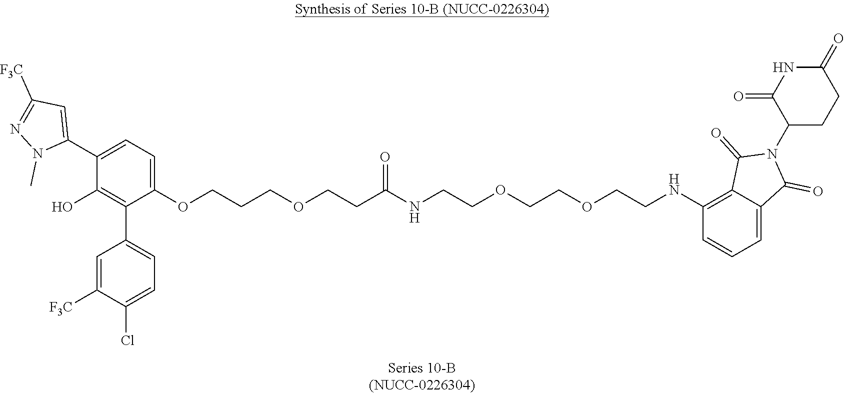 [SMILES: Cn1nc(C(F)(F)F)cc1-c1ccc(OCCCOCCC(=O)NCCOCCOCCNc2cccc3c2C(=O)N(C2CCC(=O)NC2=O)C3=O)c(-c2ccc(Cl)c(C(F)(F)F)c2)c1O]

### Synthetic Scheme

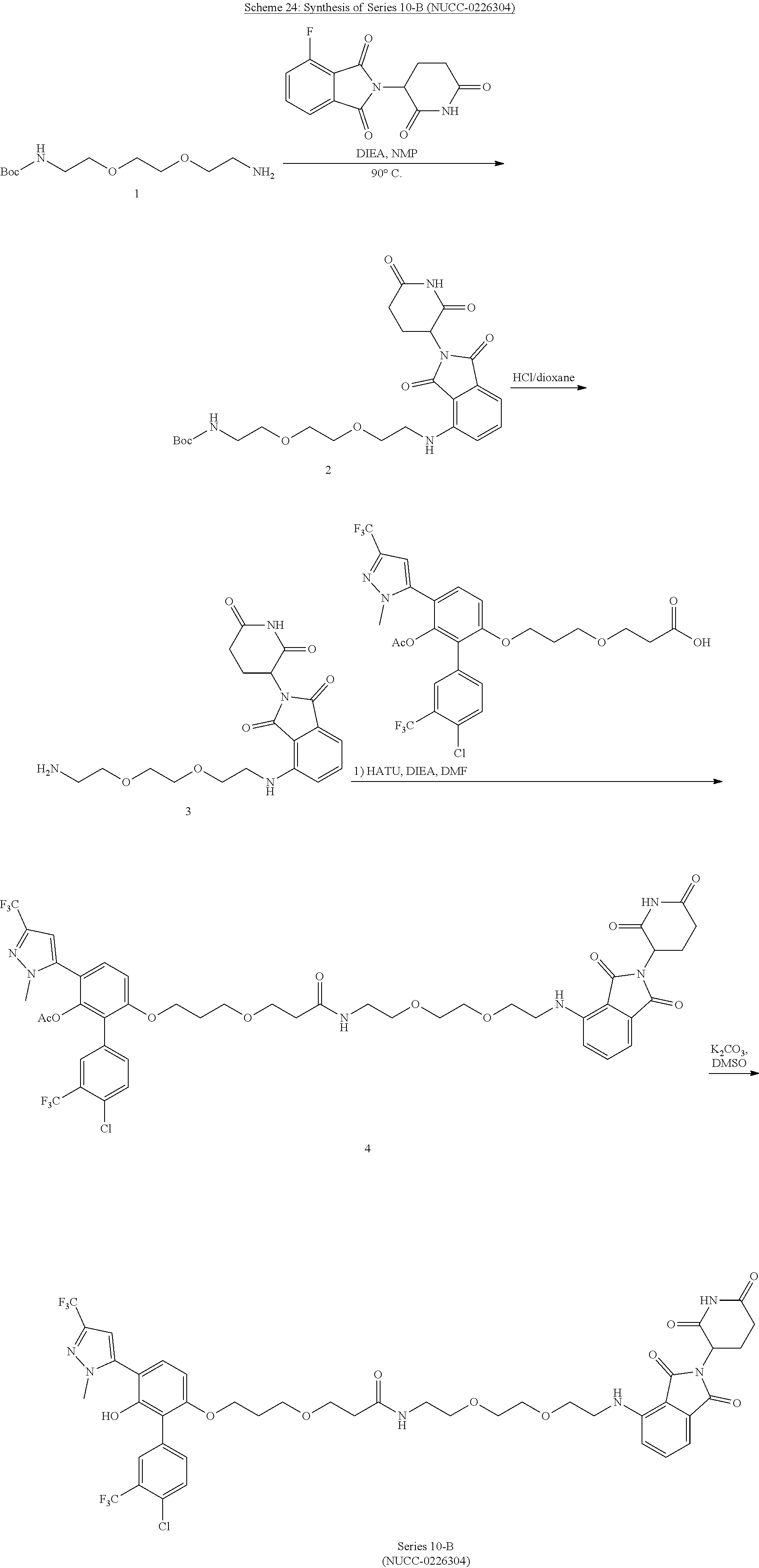

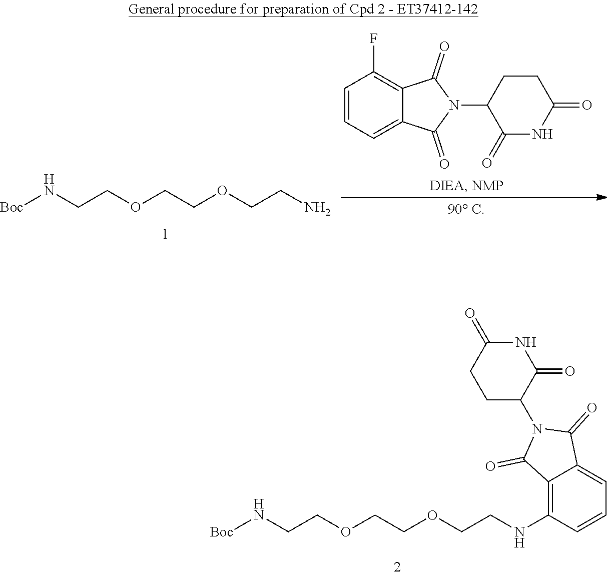 [SMILES: O=C1CCC(N2C(=O)c3cccc(F)c3C2=O)C(=O)N1 ; CC(C)(C)OC(=O)NCCOCCOCCN ; CC(C)(C)OC(=O)NCCOCCOCCNc1cccc2c1C(=O)N(C1CCC(=O)NC1=O)C2=O]

To a solution of Cpd 1 (215.76 mg, 868.87 mmol) in 1-methyl-2-pyrrolidinone (2 mL) was added N-ethyl-N-isopropylpropan-2-amine (187.16 mg, 1.45 mmol) and 2-(2,6-dioxo-3-piperidyl)-4-fluoro-isoindoline-1,3-dione (200 mg, 724.06 mmol). The reaction was stirred at 90° C. for 12 h. TLC (petroleum ether/ethyl acetate=1:1, RF=0.23) showed the reaction was completed. The reaction was poured into water (20 mL) and the reaction was extracted with ethyl acetate (20 mL). The organic layer was concentrated to give a residue which was purified by silica gel chromatography (column height: 250 mm, diameter: 100 mm, 100-200 mesh silica gel, Petroleum ether/Ethyl acetate=1/0, 0/1) to give Cpd 2 (150 mg, 41% yield) as colorless oil.

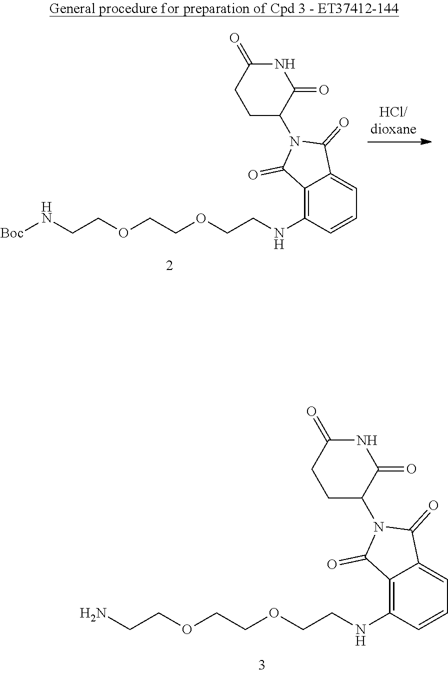 [SMILES: CC(C)(C)OC(=O)NCCOCCOCCNc1cccc2c1C(=O)N(C1CCC(=O)NC1=O)C2=O ; NCCOCCOCCNc1cccc2c1C(=O)N(C1CCC(=O)NC1=O)C2=O]

A solution of Cpd 2 (200 mg, 396.41 mmol) in HCl/dioxane (2 mL, 4 M) was stirred at 25° C. for 0.2 h. LCMS showed the reaction was completed. The reaction was concentrated to give Cpd 3 (120 mg, 75% yield) as colorless oil which was used to next step without any purification.

### LCMS Method:

$$LCMS ( ESI + ) : m / z = 405.1 ( M + H ) + , RT : 0.605 min .$$

5-95AB_2_min: LC/MS (The column used for chromatography was a Kinetex 5 μm EVO C18 100A. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 2.20 min 0.5% B in 0.01 min, 5-95% B (0.01-1.00 min), 95-100% B (1.00-1.80 min), 5% B in 1.81 min with a hold at 5% B for 0.39 min. The flow rate was 1.0 mL/min (0.01-1.80) 1.2 mL (1.81-2.20).

### General Procedure for Preparation of Cpd_4-ET37412-158

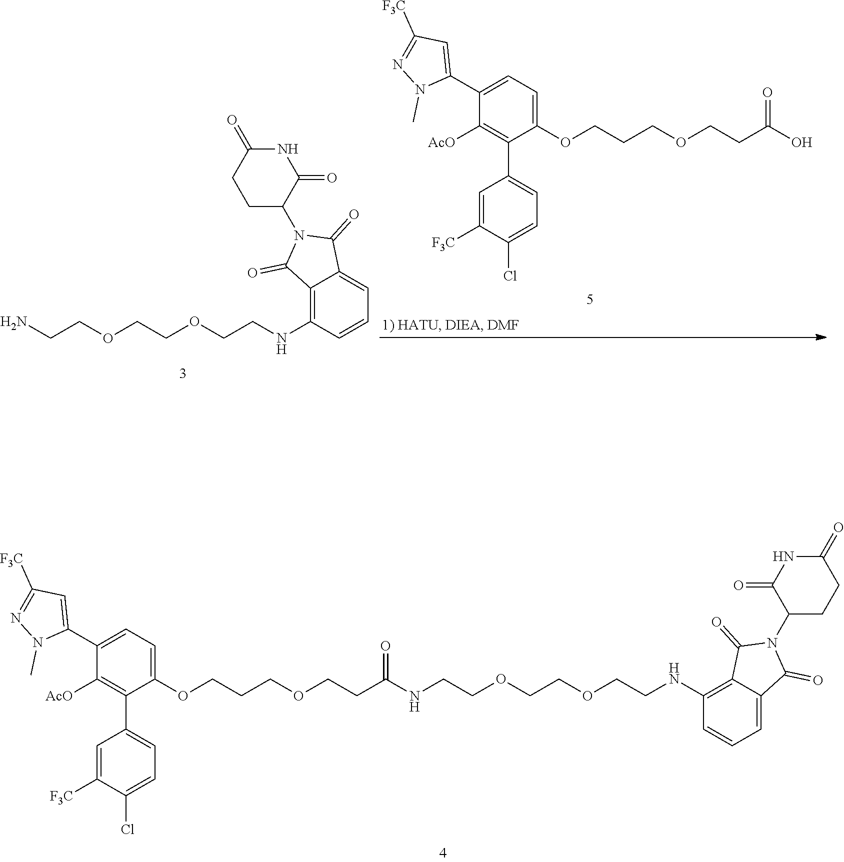 [SMILES: NCCOCCOCCNc1cccc2c1C(=O)N(C1CCC(=O)NC1=O)C2=O ; CC(=O)Oc1c(-c2cc(C(F)(F)F)nn2C)ccc(OCCCOCCC(=O)O)c1-c1ccc(Cl)c(C(F)(F)F)c1 ; CC(=O)Oc1c(-c2cc(C(F)(F)F)nn2C)ccc(OCCCOCCC(=O)NCCOCCOCCNc2cccc3c2C(=O)N(C2CCC(=O)NC2=O)C3=O)c1-c1ccc(Cl)c(C(F)(F)F)c1]

To a solution of Cpd 5 (100 mg, 164.23 mmol) and Cpd 3 (79.70 mg, 197.07 mmol) in N,N-dimethylformamide (1 mL) was added N-ethyl-N-isopropylpropan-2-amine (106.13 mg, 821 mmol) and o-(7-Azabenzotriazol-1-yl)-N,N,N′,N′-tetramethyluronium hexafluorophosphate (74.93 mg, 197.07 mmol). The reaction was stirred at 25° C. for 12 h. LCMS showed the reaction was completed. The reaction was poured into water (5 mL) and extracted with ethyl acetate (5 mL). The organic layer was concentrated to give a residue which was purified by Prep-TLC to give Cpd 4 (50 mg, 31% yield) as yellow solid.

¹H NMR: ET37412-158-g1 (400 MHz, DMSO-d₆)

δ 1.71-1.76 (m, 3H), 1.76-1.84 (m, 2H), 1.94-2.06 (m, 1H), 2.27 (t, J=6.36 Hz, 2H), 2.53-2.65 (m, 3H), 2.82-2.94 (m, 1H), 3.16 (q, J=5.71 Hz, 2H), 3.28-3.39 (m, 6H), 3.53-3.63 (m, 6H), 3.74-3.83 (m, 3H), 4.10 (br t, J=5.99 Hz, 2H), 5.05 (dd, J=12.90, 5.44 Hz, 1H), 6.59 (br s, 1H), 6.68 (s, 1H), 7.03 (d, J=7.09 Hz, 1H), 7.13 (d, J=8.56 Hz, 1H), 7.23 (d, J=8.68 Hz, 1H), 7.54-7.64 (m, 3H), 7.72 (d, J=1.47 Hz, 1H), 7.79-7.88 (m, 2H), 11.08 (s, 1H)

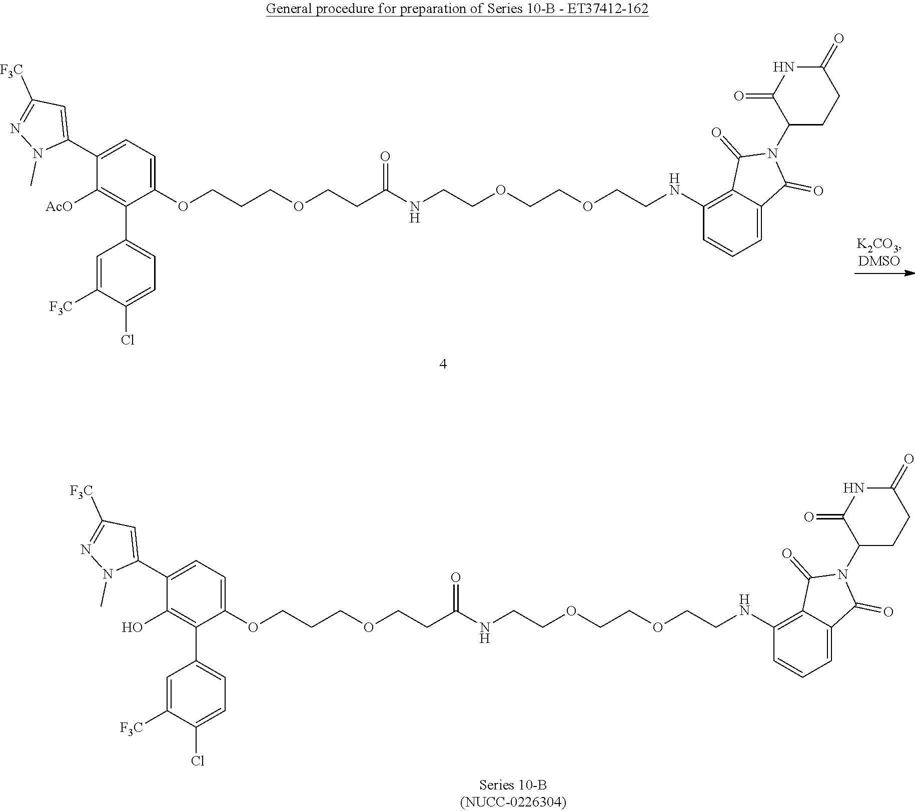 [SMILES: CC(=O)Oc1c(-c2cc(C(F)(F)F)nn2C)ccc(OCCCOCCC(=O)NCCOCCOCCNc2cccc3c2C(=O)N(C2CCC(=O)NC2=O)C3=O)c1-c1ccc(Cl)c(C(F)(F)F)c1 ; Cn1nc(C(F)(F)F)cc1-c1ccc(OCCCOCCC(=O)NCCOCCOCCNc2cccc3c2C(=O)N(C2CCC(=O)NC2=O)C3=O)c(-c2ccc(Cl)c(C(F)(F)F)c2)c1O]

To a solution of Cpd 4 (30 mg, 30.14 μmol, 1 eq) in dimethyl sulfoxide (1 mL) was added K₂CO₃ (8.33 mg, 60.28 mmol). The reaction was stirred at 80° C. for 12 h. The reaction was purified by Prep-HPLC column: mobile phase: [water (0.1% 2,2,2-trifluoroacetic acid)-acetonitrile]; B %: 50%-80%, 9 min to give Series 10-B (20 mg, 60% yield) as white solid.

¹H NMR: ET37412-162-1 (400 MHz, CDCl₃-d₆)

δ 1.87-1.96 (m, 2H), 2.08-2.19 (m, 1H), 2.44-2.57 (m, 2H), 2.68-2.77 (m, 2H), 2.82-2.91 (m, 1H), 3.41-3.48 (m, 6H), 3.55-3.67 (m, 9H), 3.68-3.74 (m, 3H), 3.84 (s, 3H), 4.04 (br t, J=6.02 Hz, 2H), 4.89 (br dd, J=12.04, 5.36 Hz, 1H), 6.57 (s, 1H), 6.69 (d, J=8.58 Hz, 1H), 6.90 (br d, J=8.46 Hz, 2H), 7.09-7.14 (m, 1H), 7.21 (d, J=8.46 Hz, 1H), 7.47-7.54 (m, 2H), 7.60-7.64 (m, 1H), 7.75 (s, 1H), 8.63 (br s, 1H)

$$LCMS ( ESI + ) : m / z = 953.3 {( M + H )}^{+} , RT : 2.831 min .$$

5_95AB_6min_MS1500-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1500.

Final Report of NUCC-0226504 (ET42365-21-1), NUCC-0226503 (ET42365-19-1), NUCC-0226499 (ET42365-26-1), NUCC-0226498 (ET42365-25-1), NUCC-0226497 (ET42365-23-1) and NUCC-0226496 (ET42365-20-1)

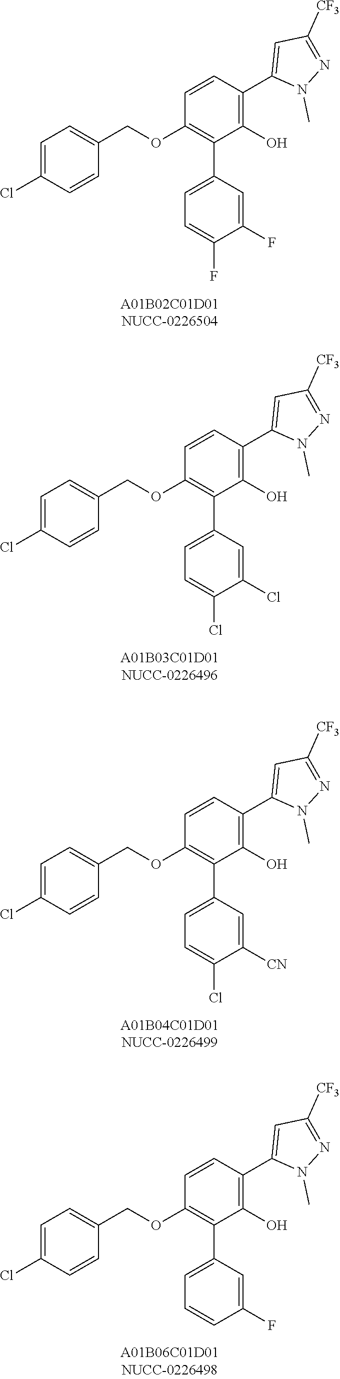 [SMILES: Cn1nc(C(F)(F)F)cc1-c1ccc(OCc2ccc(Cl)cc2)c(-c2ccc(F)c(F)c2)c1O ; Cn1nc(C(F)(F)F)cc1-c1ccc(OCc2ccc(Cl)cc2)c(-c2ccc(Cl)c(Cl)c2)c1O ; Cn1nc(C(F)(F)F)cc1-c1ccc(OCc2ccc(Cl)cc2)c(-c2ccc(Cl)c(C#N)c2)c1O ; Cn1nc(C(F)(F)F)cc1-c1ccc(OCc2ccc(Cl)cc2)c(-c2cccc(F)c2)c1O]

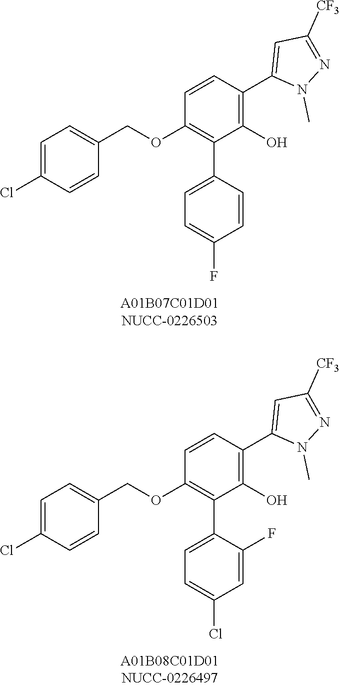 [SMILES: Cn1nc(C(F)(F)F)cc1-c1ccc(OCc2ccc(Cl)cc2)c(-c2ccc(F)cc2)c1O ; Cn1nc(C(F)(F)F)cc1-c1ccc(OCc2ccc(Cl)cc2)c(-c2ccc(Cl)cc2F)c1O]

### Synthetic Scheme: Preparation of A01B02C01D01 (NUCC-0226504), A01B07C01D01 (NUCC-0226503), A01B04C01D01 (NUCC-0226499), A01B06C01D01 (NUCC-0226498), A01B08C01D01 (NUCC-0226497) and A01B03C01D01 (NUCC-0226496)

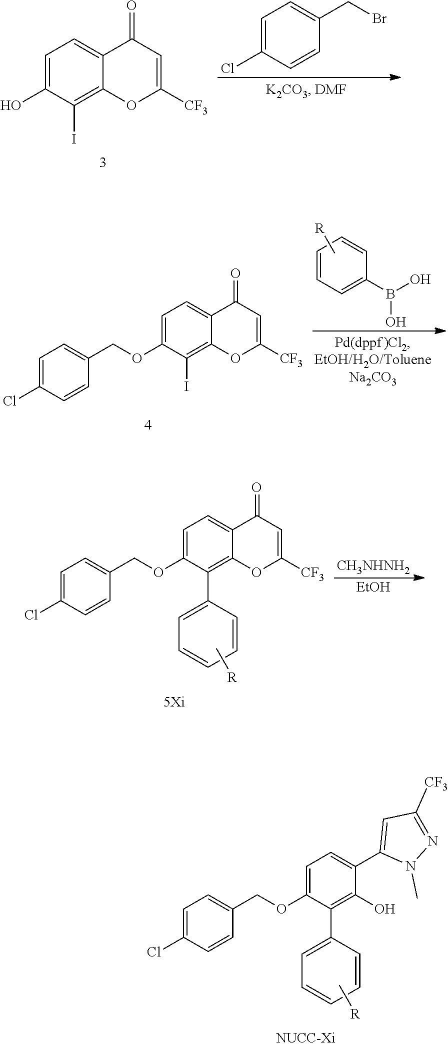

Take A01B03C01D01 (NUCC-0226496) as an example.

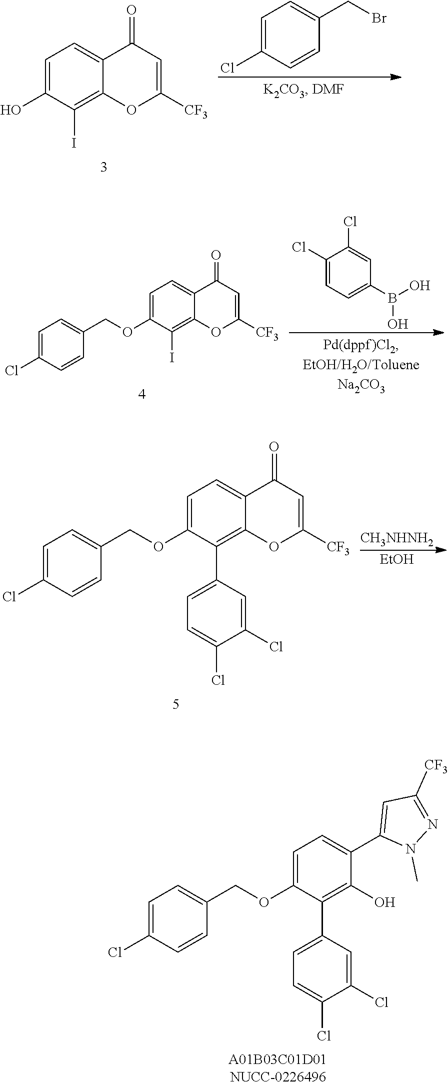

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (Sum particles). Detection methods are diode array (DAD) and evaporative light scattering (ELSD) detection. MS mode was positive electrospray ionization. MS range was 100-1000.

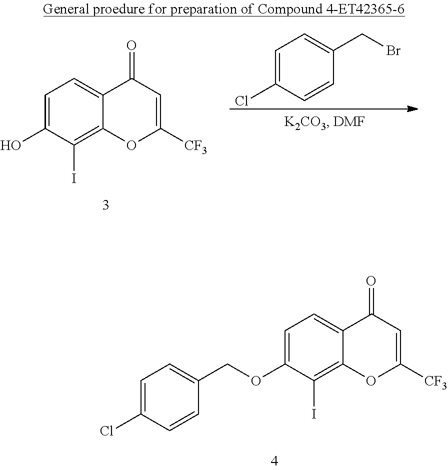 [SMILES: O=c1cc(C(F)(F)F)oc2c(I)c(O)ccc12 ; Clc1ccc(CBr)cc1 ; O=c1cc(C(F)(F)F)oc2c(I)c(OCc3ccc(Cl)cc3)ccc12]

To a solution of Compound 3 (2.5 g, 7.02 mmol, 1 eq) in DMF (50 mL) were added K₂CO₃ (1.94 g, 14.04 mmol, 2 eq) and 1-(bromomethyl)-4-chloro-benzene (1.73 g, 8.43 mmol, 1.2 eq), the reaction was stirred at 30° C. for 8 hrs. LCMS showed the starting material was consumed and a new peak with desired product Ms was detected. The reaction was diluted with water (150 mL), the precipitate was filtered and dried under high vacuum to give Compound 4 (2 g, yield 56.3%) as a white solid. The product was used to the next step directly without further purification.

$$LCMS ( ESI + ) : RT : 1.144 min , m / z = 480.8 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 ml/min.

### General Procedure for Preparation of Compound 5-ET42365-10

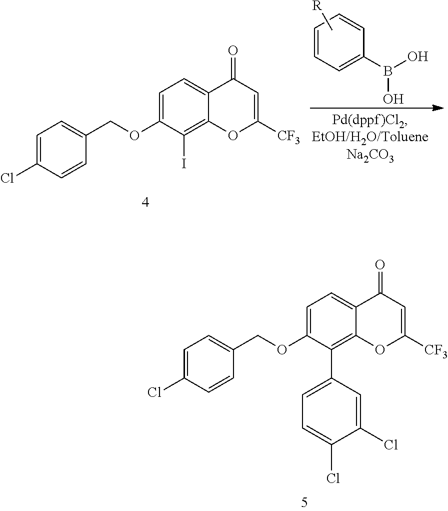

To a solution of Compound 4 (200 mg, 416.14 μmol, 1 eq), Na₂CO₃ (88.21 mg, 832.29 μmol, 2 eq) and (3,4-dichlorophenyl)boronic acid (95.29 mg, 499.37 μmol, 1.2 €q) in a mixture solution of toluene (6 mL), EtOH (1.2 mL) and H₂O (0.3 mL) was added Pd(dppf)Cl₂·CH₂Cl₂ (33.98 mg, 41.61 μmol, 0.1 eq) under nitrogen, the reaction was stirred at 80° C. for 12 hrs. LCMS showed the starting material was consumed and a new peak with desired product Ms was detected. The reaction was filtered and the filtrate was concentrated to give the crude product. The crude product was purified by Prep-TLC (petroleum ether/ethyl acetate=2/1) to give Compound 5 (150 mg, yield 64.92%) as a yellow solid.

$$LCMS ( ESI + ) : RT : 0.936 min , m / z = 499.1 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

### General Procedure for Preparation of A01B03C01D01-ET42365-20

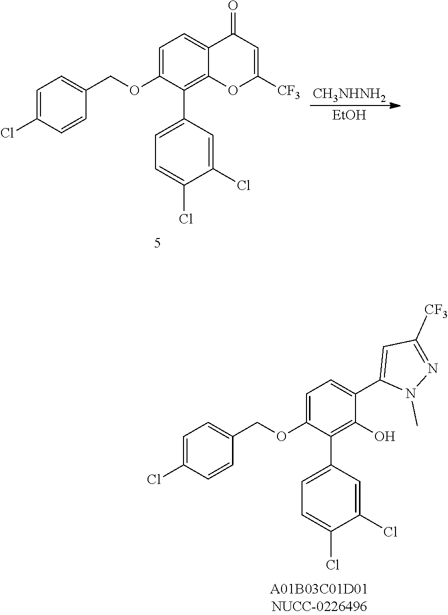

To a solution of Compound 5 (150 mg, 300.18 μmol, 1 eq) in EtOH (2.5 mL) was added CH₃NHNH₂ (150 mg, 1.30 mmol, 171.43 μL, 40% purity, 4.34 eq), the reaction was stirred at 80° C. for 16 hrs. LCMS showed the starting material was consumed and two new peaks with desired product Ms were detected. The reaction was concentrated to give crude product. The crude product was purified by Prep-HPLC (column: Phenomenex luna C18 100*40 mm*5 um; mobile phase: [water (0.1% TFA)-ACN]; B %: 55%-85%, 8 min), then (column: Waters Xbridge BEH C18 100*30 mm*10 um; mobile phase: [water (10 mM NH₄HCO₃)-ACN]; B %: 60%-90%, 8 min) to give A01B03C01D01 (42.2 mg, yield 26.64%) as white solid.

¹H NMR: (ET42365-20-P1X, 400 MHz, CHLOROFORM-d) δ 3.84 (s, 3H), 5.07 (s, 2H), 5.15 (s, 1H), 6.55 (s, 1H), 6.70 (d, J=8.51 Hz, 1H), 7.19 (t, J=8.88 Hz, 3H), 7.26 (d, J=1.88 Hz, 1H), 7.33 (d, J=8.38 Hz, 2H), 7.55 (d, J=1.88 Hz, 1H), 7.59 (d, J=8.25 Hz, 1H)

$$LCMS ( ESI + ) : RT : 2.889 min , m / z = 527.1 , 529. {( M + H , M + 2 + H )}^{+} .$$

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (Sum particles). Detection methods are diode array (DAD) and evaporative light scattering (ELSD) detection. MS mode was positive electrospray ionization. MS range was 100-1000.

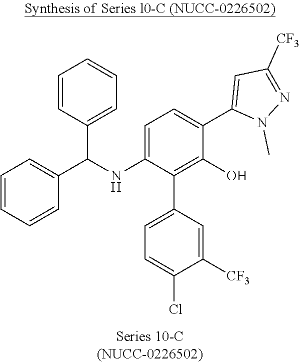 [SMILES: Cn1nc(C(F)(F)F)cc1-c1ccc(NC(c2ccccc2)c2ccccc2)c(-c2ccc(Cl)c(C(F)(F)F)c2)c1O]

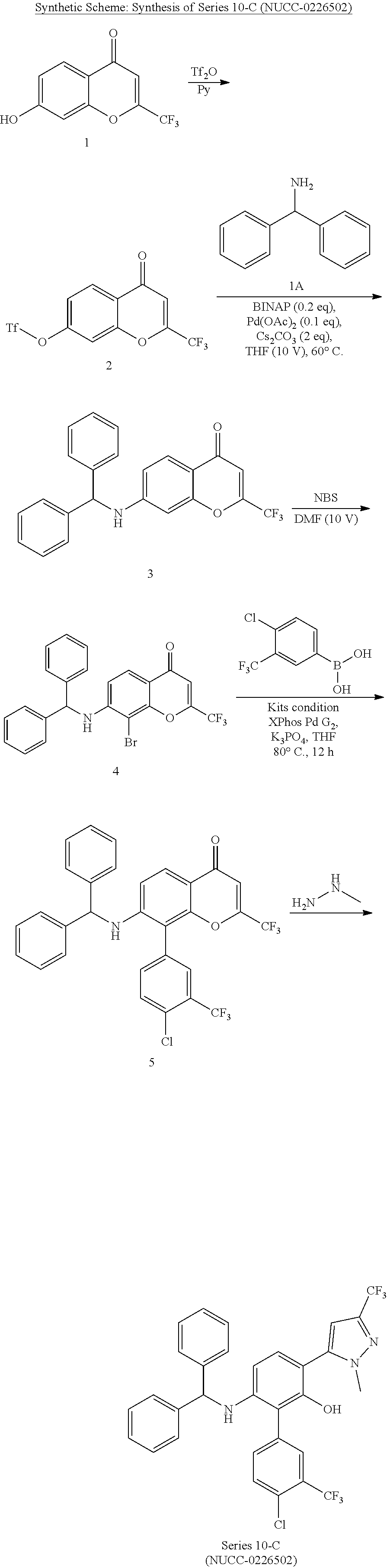

### Experiments for Largest Scale Run

### General Procedure for Preparation of Cpd 2-ET37413-173

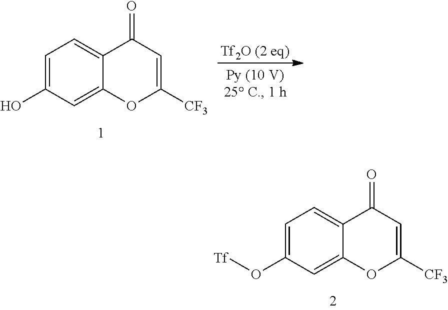

To a solution of Cpd 1 (1 g, 4.35 mmol) in pyridine (10 ml) was added trifluoromethanesulfonic anhydride (2.45 g, 8.69 mmol). The mixture was stirred at 25° C. for 1 h. LCMS showed the reaction was completed. The reaction was poured into water (10 mL) and extracted with ethyl acetate (10 ml . . . ). The organic layer was concentrated to give Cpd 2 (1.1 g, yield 70%) as yellow oil which was used to next step directly.

### LCMS Method:

$$LCMS ( ESI + ) : m / z = 363.1 ( M + H ) + , RT : 0.837 min .$$

5-95AB_2_min: LC/MS (The column used for chromatography was a Kinetex 5 μm EVO C18 100A. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 2.20 min 0.5% B in 0.01 min, 5-95% B (0.01-1.00 min), 95-100% B (1.00-1.80 min), 5% B in 1.81 min with a hold at 5% B for 0.39 min. The flow rate was 1.0 mL/min (0.01-1.80) 1.2 mL (1.81-2.20).

### General Procedure for Preparation of Cpd 3-ET37412-183

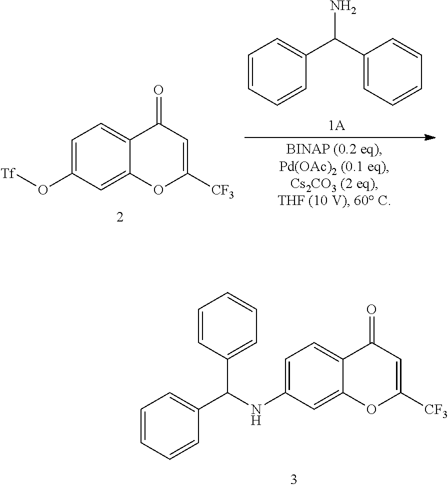

To a solution of Cpd 2 (1 g, 2.76 mmol) in tetrahydrofuran (10 mL) was added palladium (II) acetate (61.98 mg, 276.09 μmol), diphenylmethanamine (1.01 g, 5.52 mmol), Cs₂CO₃ (1.80 g, 5.52 mmol), (±)-2,2′-Bis(diphenylphosphino)-1, l′-binaphthalene (343.83 mg, 552.18 μmol, 0.2 eq) under N₂. The mixture was stirred at 60° C. for 12 h. LCMS showed the reaction was completed. The reaction was concentrated to give a residue. The residue was purified by silica gel chromatography (column height: 250 mm, diameter: 100 mm, 100-200 mesh silica gel, petroleum ether/ethyl acetate=1/0, 0/1) to give Cpd 3 (0.4 g, yield 37%) as yellow solid.

¹H NMR: ET37412-183-1 (400 MHz, CDCl₃)

δ 5.00 (br s, 1H), 5.65 (br d, J=3.30 Hz, 1H), 6.39 (d, J=2.20 Hz, 1H), 6.57 (s, 1H), 6.71 (dd, J=8.80, 2.20 Hz, 1H), 7.31-7.41 (m, 10H), 7.94 (d, J=8.80 Hz, 1H)

### General Procedure for Preparation of Cpd 4-ET37412-214

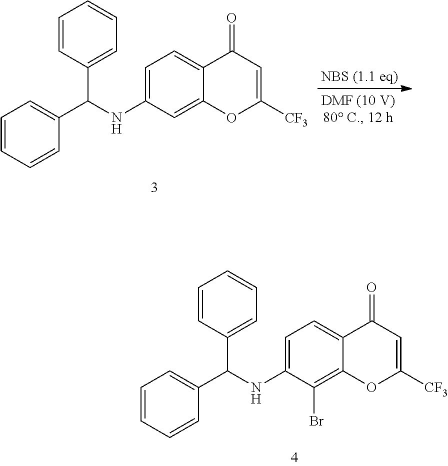 [SMILES: O=c1cc(C(F)(F)F)oc2cc(NC(c3ccccc3)c3ccccc3)ccc12 ; O=c1cc(C(F)(F)F)oc2c(Br)c(NC(c3ccccc3)c3ccccc3)ccc12]

To a solution of Cpd 3 (330 mg, 834.65 μmol) in N,N-dimethylformamide (3 ml) was added N-Bromosuccinimide (163.41 mg, 918.12 μmol). The reaction was stirred at 80° C. for 12 h. LCMS showed the reaction was completed. The reaction was poured into water (10 mL) and extracted with ethyl acetate (10 mL). The combined organic phase was dried with anhydrous Na₂SO₄, filtered and concentrated in vacuum. The residue was purified by silica gel chromatography (column height: 250 mm, diameter: 100 mm, 100-200 mesh silica gel, petroleum ether/ethyl acetate=1/0, 0/1) to afford Cpd 4 (200 mg, yield 51%) as white solid.

$$LCMS ( ESI + ) : m / z = 476.2 {( M + H )}^{+} , RT : 0.935 min .$$

### General Procedure for Preparation of Cpd 5. NotebookPage: ET37412-234

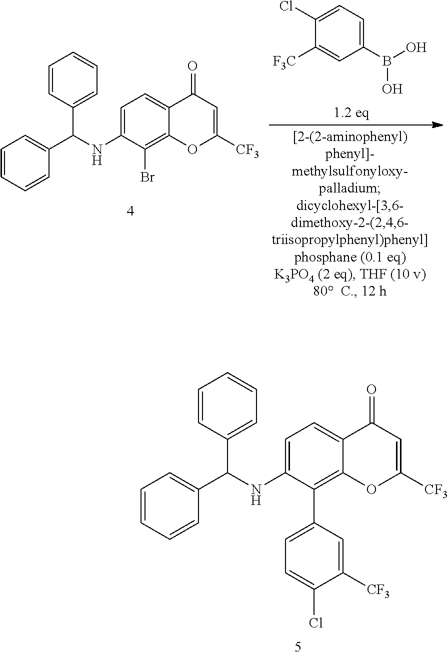 [SMILES: O=c1cc(C(F)(F)F)oc2c(Br)c(NC(c3ccccc3)c3ccccc3)ccc12 ; OB(O)c1ccc(Cl)c(C(F)(F)F)c1 ; O=c1cc(C(F)(F)F)oc2c(-c3ccc(Cl)c(C(F)(F)F)c3)c(NC(c3ccccc3)c3ccccc3)ccc12]

To solution of Cpd 4 (0.2 g, 0.421 μmol) in tetrahydrofuran (2 mL) was added (4-chloro-3-(trifluoromethyl)phenyl)boronic acid (0.113 g, 0.506 μmol), [2-(2-aminophenyl)phenyl]-methylsulfonyloxy-palladium;dicyclohexyl-[3,6-dimethoxy-2-(2,4,6-triisopropylphenyl)phenyl]phosphane (38 mg, 0.042 μmol) and potassium phosphate (179 mg, 0.843 μmol) at 25° C. And the reaction was stirred at 80° C. for 12 h. The reaction was poured into water (5 mL). The organic phase was separated and the aqueous phase was extracted with ethyl acetate (5 mL) for three times. The organic phase was washed with brine (10 mL), dried over Na₂SO₄, filtered and concentrated under vacuum to give crude product. The crude product was purified by column chromatography on silica gel (petroleum ether/ethyl acetate=1/0 to 1/2) to give Cpd 5 (0.1 g, yield 41%) as a colorless oil.

$$LCMS : m / z = 574.4 {( M + H )}^{+} , RT : 1.211 min$$

5-95AB_2_min: LC/MS (The column used for chromatography was a Kinetex 5 μm EVO C18 100A 2.1*30 mm. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 1.50 min. 5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

### General Procedure for Preparation of Series 10-C. NotebookPage: ET37412-246

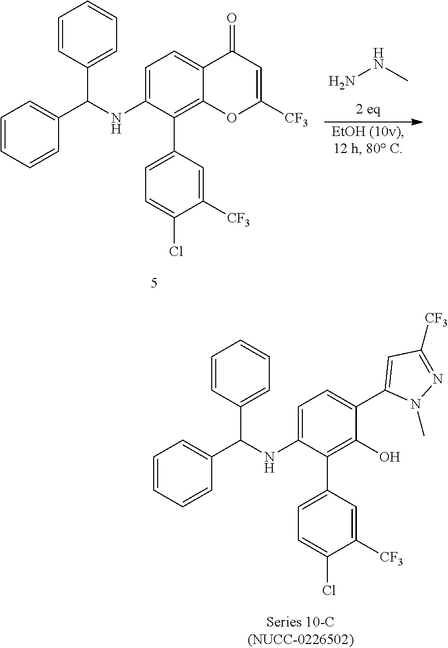

To a solution of Cpd 5 (0.1 g, 0.174 μmol) in ethyl alcohol (1 mL) was added diazomethane (60 mg, 0.34 μmol) at 25° C. And the reaction was stirred at 80° C. for 12 h. The reaction was poured into water (5 mL). The mixture was extracted with ethyl acetate (5 mL) for three times. The organic phase was washed with brine (10 mL), dried over Na₂SO₄, filtered and concentrated under vacuum to give crude product which was purified by Prep-HPLC (column: Waters Xbridge Prep OBD C18 150*40 mm*10 um; mobile phase: [water (10 mM NH₄HCO₃)-acetonitrile]; B %: 60%-90%, 8 min) to give Series 10-C (7 mg, 15% yield) as white solid.

¹H NMR (ET37412-246-P1A5, 400 MHz, DMSO-d₆)

δ 3.71 (s, 3H), 4.86 (d, J=5.95 Hz, 1H), 5.67 (d, J=5.73 Hz, 1H), 6.27 (d, J=8.60 Hz, 1H), 6.60 (s, 1H), 6.99 (d, J=8.38 Hz, 1H), 7.22 (td, J=5.68, 2.54 Hz, 2H), 7.26-7.34 (m, 8H), 7.72 (dd, J=8.38, 1.54 Hz, 1H), 7.81 (dd, J=4.85, 3.31 Hz, 2H), 8.53 (s, 1H)

LCMS: m/z=602.2 (M+H)⁺, RT: 3.023 min

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

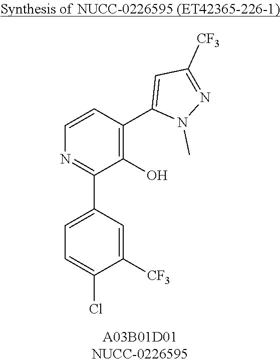 [SMILES: Cn1nc(C(F)(F)F)cc1-c1ccnc(-c2ccc(Cl)c(C(F)(F)F)c2)c1O]

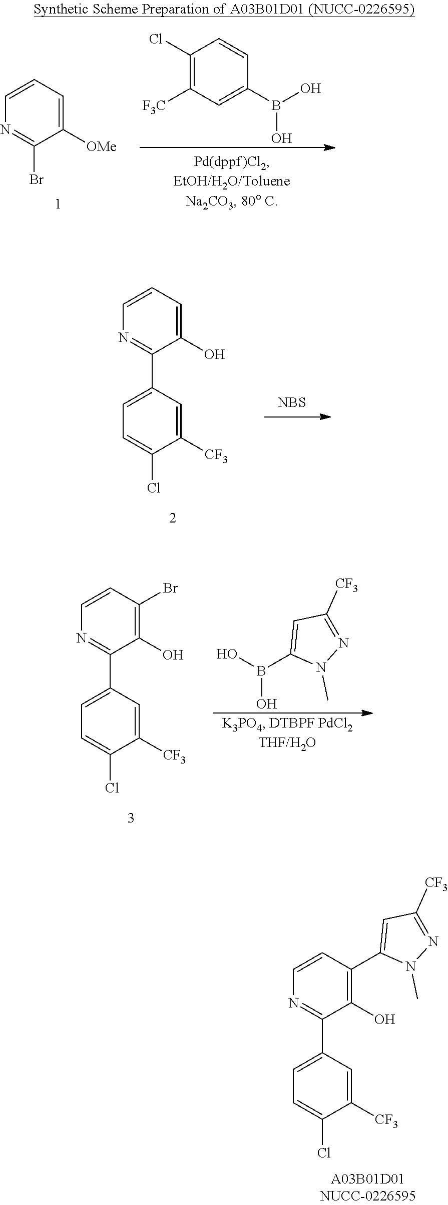

### Chemical Synthesis

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% trifluoroacetic acid in water, mobile phase B was 0.018% trifluoroacetic acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

### Experimentals for Largest Scale Run

### General Procedure for Preparation of Compound 2-ET43365-193

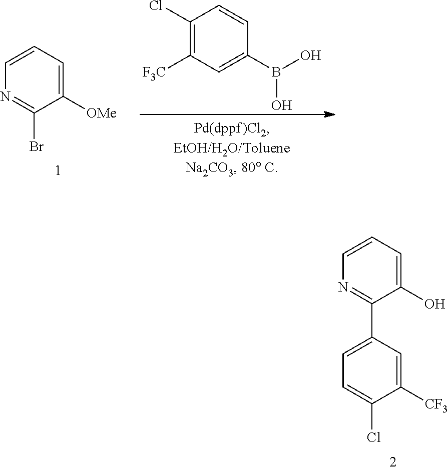 [SMILES: COc1cccnc1Br ; OB(O)c1ccc(Cl)c(C(F)(F)F)c1 ; Oc1cccnc1-c1ccc(Cl)c(C(F)(F)F)c1]

To a solution of Compound 1 (1 g, 5.75 mmol, 1 eq), [4-chloro-3-(trifluoromethyl)phenyl]-boronic acid (1.94 g, 8.63 mmol, 1.5 eq) and Na₂CO₃ (1.22 g, 11.50 mmol, 2 eq) in a mixture solution of toluene (30 mL), EtOH (6 mL) and H₂O (1.2 mL) was added Pd(dppf)Cl₂·CH₂Cl₂ (469.35 mg, 575.00 μmol, 0.1 eq) under nitrogen, the reaction was stirred at 100° C. for 2 hrs. LCMS showed the starting material was consumed and a new peak with desired product Ms was detected. The reaction was filtered and the filtrate was concentrated to give the crude product. The crude product diluted with water (60 mL) and extracted with ethyl acetate (3×50 mL). The organic layer was washed with aqueous K₂CO₃ (2×60 mL) and brine (60 mL), dried over anhydrous sodium sulfate, filtered and concentrated under reduced pressure to afford Compound 2 (0.5 g, yield 28.6%) as a yellow solid. The crude product was used to the next step directly without further purification.

¹H NMR (ET42365-193-P1A, 400 MHz, CHLOROFORM-d) δ 7.21-7.25 (m, 1H), 7.28-7.31 (m, 1H), 7.60 (d, J=8.25 Hz, 1H), 8.08 (dd, J=8.32, 1.69 Hz, 1H), 8.33 (d, J=2.25 Hz, 2H)

### General Procedure for Preparation of Compound 3-ET42365-207

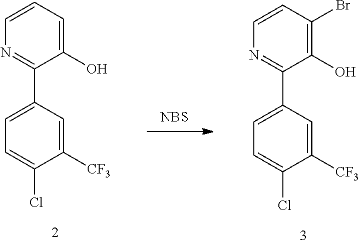 [SMILES: Oc1cccnc1-c1ccc(Cl)c(C(F)(F)F)c1 ; "NBS" ; Oc1c(Br)ccnc1-c1ccc(Cl)c(C(F)(F)F)c1]

To a solution of Compound 2 (400 mg, 1.46 mmol, 1 eq) in DMF (4 mL) was added NBS (312.21 mg, 1.75 mmol, 1.2 eq) in portions at 0° C., the reaction was stirred at 20° C. for 3 hrs. LCMS showed about 30% of the starting material was remaining and a new peak (about 35%) with desired product Ms was detected. The reaction was diluted with water (20 mL) and was extracted with ethyl acetate (3×15 mL). The combined organic layer were washed with brine (20 mL) and dried over Na₂SO₄ and concentrated to give crude product. The crude product was purified by Prep-HPLC (column: Phenomenex Gemini-NX 80*40 mm*3 um; mobile phase: [water (10 mM NH₄HCO₃)-ACN], B %: 30%-60%, 8 min) to give Compound 3 (120 mg, yield 23.29%) as a yellow solid.

$$LCMS ( ESI + ) : RT : 0.833 min , m / z 352. , 354. {( M + H )}^{+} .$$

5-95AB 2 min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 ml/min.

### General Procedure for Preparation of A03B01D01-ET42365-226

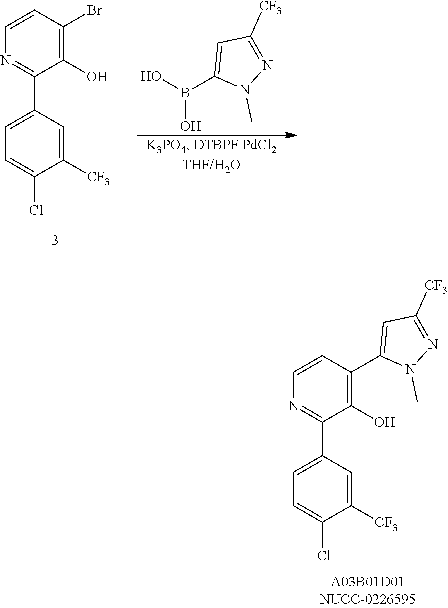

To a solution of Compound 3 (120 mg, 340.39 μmol, 1 eq), [2-methyl-5-(trifluoromethyl)-pyrazol-3-yl]boronic acid (85.81 mg, 442.51 μmol, 1.3 eq) and K₃PO₄ (144.51 mg, 680.79 μmol, 2 eq) in a mixture solution of H₂O (1 mL) and THF (5 mL) was added ditert-butyl(cyclopentyl)phosphane;dichloropalladium;iron (22.18 mg, 34.04 μmol, 0.1 eq) under nitrogen atmosphere, the reaction was stirred at 80° C. for 12 hrs. LCMS showed the starting material was consumed and a new peak with desired product Ms was detected. The reaction was filtered and the filtrate was concentrated to give the crude product. The crude product was purified by Prep-HPLC (column: Waters Xbridge Prep OBD C18 150*40 mm*10 um; mobile phase: [water (10 mM NH₄HCO₃)-ACN]; B %: 65%-95%, 8 min) to give A03B01D01 (26.8 mg, yield 18.37%) as a white solid.

¹H NMR (ET42365-257-P1A, 400 MHz, CHLOROFORM-d) δ 4.28 (s, 3H), 5.50 (br s, 1H), 6.81 (s, 1H), 7.36 (d, J=8.38 Hz, 1H), 7.53 (d, J=8.38 Hz, 1H), 7.65 (d, J=8.50 Hz, 1H), 8.14-8.21 (m, 1H), 8.43 (s, 1H)

$$LCMS ( ESI + ) : RT : 3.535 min , m / z 422.1 {( M + H )}^{+} .$$

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% trifluoroacetic acid in water, mobile phase B was 0.018% trifluoroacetic acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

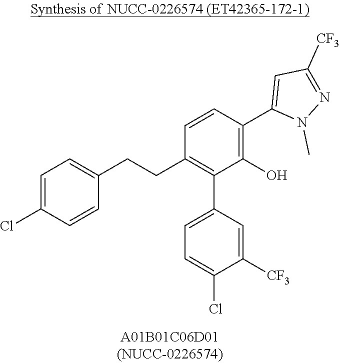 [SMILES: Cn1nc(C(F)(F)F)cc1-c1ccc(CCc2ccc(Cl)cc2)c(-c2ccc(Cl)c(C(F)(F)F)c2)c1O]

### Synthetic Scheme

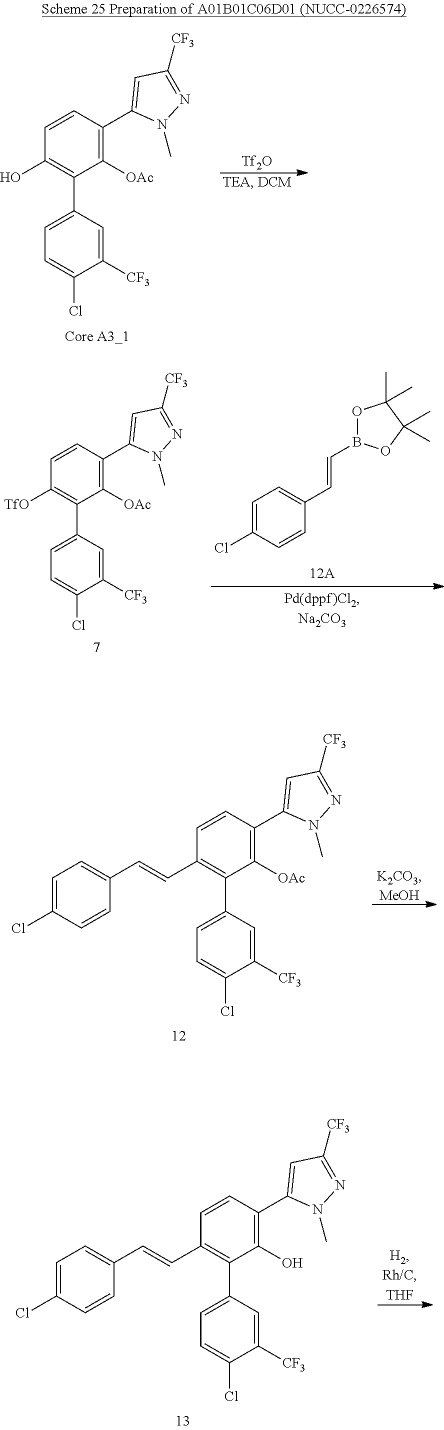

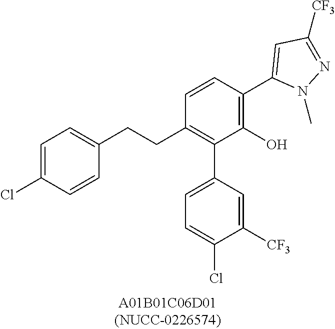 [SMILES: Cn1nc(C(F)(F)F)cc1-c1ccc(CCc2ccc(Cl)cc2)c(-c2ccc(Cl)c(C(F)(F)F)c2)c1O]

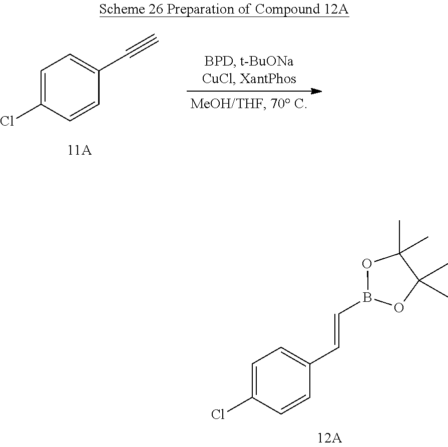

### Chemical Synthesis

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

50_100AB_6min-220-254-ELSD: LC/MS (The gradient was 50% B in 0.40 min and 50-100% B at 0.40-3.00 min, hold on 100% B for 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1000.

### Experimentals for Largest Scale Run

### General Procedure for Preparation of Compound 7-ET43365-132

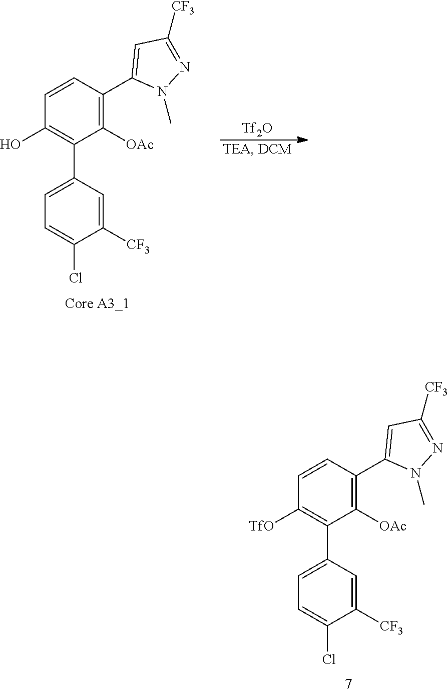 [SMILES: "Tf2O" ; CC(=O)Oc1c(-c2cc(C(F)(F)F)nn2C)ccc(O)c1-c1ccc(Cl)c(C(F)(F)F)c1 ; "TEA, DCM" ; CC(=O)Oc1c(-c2cc(C(F)(F)F)nn2C)ccc(OS(=O)(=O)C(F)(F)F)c1-c1ccc(Cl)c(C(F)(F)F)c1]

To a solution of Core A3_1 (600 mg, 1.25 mmol, 1 eq) and pyridine (396.51 mg, 5.01 mmol, 404.61 μL, 4 eq) in DCM (20 mL) was added Tf₂O (530.37 mg, 1.88 mmol, 310.16 μL, 1.5 eq) dropwise at 0° C., the reaction was stirred at 20° C. for 12 hrs. TLC (Petroleum ether/Ethyl acetate=2/1) showed the starting material was consumed and a new peak was generated. The reaction was diluted with water (60 mL) and extracted with DCM (3×30 mL). The organic layer was separated and the combined organic layer was washed with brine (60 mL), dried over Na₂SO₄, filtered and concentrated under reduced pressure to give crude product. The crude product was purified by column chromatography on silica gel (eluted with petroleum ether/ethyl acetate=50/1 to 10/1) to give Compound 7 (600 mg, yield 74.46%) as a white solid.

¹H NMR (ET42365-132-P1A, 400 MHz, CHLOROFORM-d) δ 1.80 (s, 3H), 3.85 (s, 3H), 6.57 (s, 1H), 7.47 (dd, J=8.25, 1.75 Hz, 1H), 7.49-7.56 (m, 2H), 7.64 (d, J=8.25 Hz, 1H), 7.69 (d, J=1.63 Hz, 1H)

### General Procedure for Preparation of Compound 12A-ET42365-142

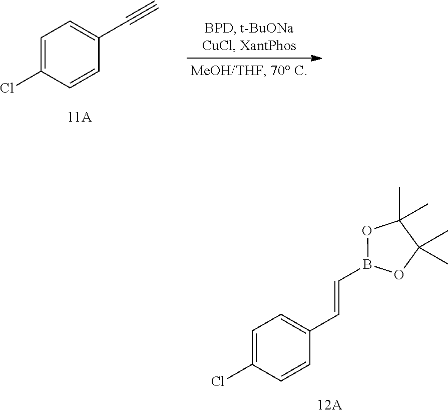

To a solution of Compound 11 (500 mg, 3.66 mmol, 1 eq), 4,4,5,5-tetramethyl-2-(4,4,5,5-tetramethyl-1,3,2-dioxaborolan-2-yl)-1,3,2-dioxaborolane (1.02 g, 4.03 mmol, 1.1 eq) and NaOtBu (703.65 mg, 7.32 mmol, 2 eq) in THF (10 mL) were added MeOH (234.60 mg, 7.32 mmol, 296.29 μL, 2 eq), Xantphos (317.74 mg, 549.14 μmol, 0.15 eq) and CuCl (10.87 mg, 109.83 μmol, 2.63 μL, 0.03 eg) under nitrogen. The mixture was stirred at 70° C. for 1 hour. HPLC showed about 19% of the starting material was remaining and a new peak was detected. The reaction was diluted with water (60 mL) and was extracted with ethyl acetate (3×50 mL). The combined organic layers were washed with brine (60 mL), dried over Na₂SO₄ and concentrated to give crude product, which was purified by column chromatography on silica gel (eluted with petroleum ether/ethyl acetate=100/1 to 20/1) to give Compound 12A (270 mg, yield 22.3%) as a yellow solid.

¹H NMR (ET42365-142-P1A, 400 MHz, CHLOROFORM-d) δ 1.22 (s, 12H), 6.04 (d, J=18.39 Hz, 1H), 7.16-7.24 (m, 3H), 7.29-7.36 (m, 2H)

### General Procedure for Preparation of Compound 12-ET42365-151

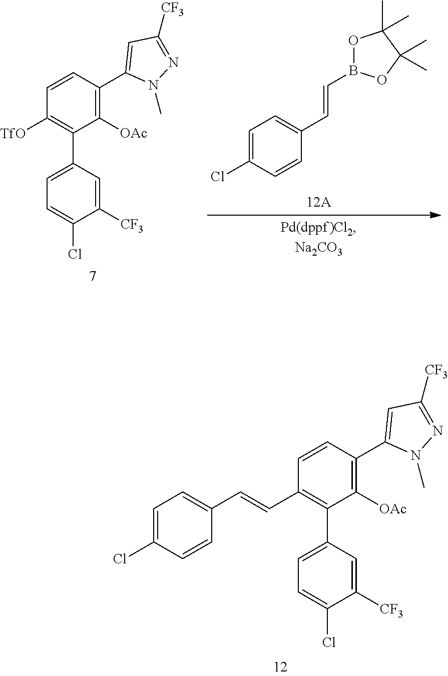 [SMILES: CC(=O)Oc1c(-c2cc(C(F)(F)F)nn2C)ccc(OS(=O)(=O)C(F)(F)F)c1-c1ccc(Cl)c(C(F)(F)F)c1 ; CC1(C)OB(/C=C/c2ccc(Cl)cc2)OC1(C)C ; CC(=O)Oc1c(-c2cc(C(F)(F)F)nn2C)ccc(/C=C/c2ccc(Cl)cc2)c1-c1ccc(Cl)c(C(F)(F)F)c1]

To a solution of Compound 7 (200 mg, 327.42 μmol, 1 eq), Compound 12A (216.55 mg, 654.84 μmol, 80% purity, 2 eq) and Na₂CO₃ (69.41 mg, 654.84 μmol, 2 eq) in a mixture solution of dioxane (5 mL) and H₂O (1 mL) was added Pd(dppf)Cl₂·CH₂Cl (26.74 mg, 32.74 μmol, 0.1 eq) under nitrogen, the reaction was stirred at 85° C. for 12 hrs. LCMS showed the starting material was consumed and a new peak with desired product Ms was detected. The reaction was filtered and the filtrate was concentrated to give the crude product. The crude product was purified by Prep-TLC (petroleum ether/ethyl acetate=2/1) to give Compound 12 (35 mg, yield 14.27%) as a yellow solid.

$$LCMS ( ESI + ) \text{:} RT = 0.971 min , m / z 599.2 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Compound 13-ET42365-154

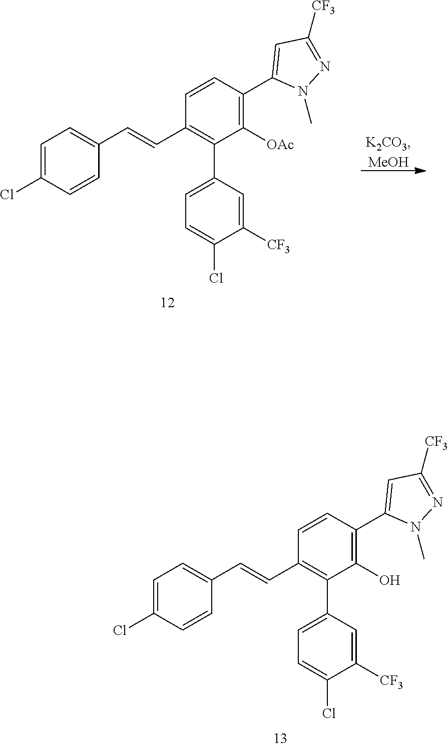

To a solution of Compound 12 (35 mg, 46.72 μmol, 80% purity, 1 eq) in MeOH (2 mL) was added K₂CO₃ (12.91 mg, 93.43 μmol, 2 eq), the reaction was stirred at 20° C. for 3 hrs. LCMS showed the starting material was consumed and a new peak with desired product Ms was detected. The reaction was diluted with water (10 mL) and was extracted with ethyl acetate (3×10 mL). The combined organic layers were washed with brine (10) mL), dried over Na₂SO₄ and concentrated to give crude Compound 13 (18 mg, yield 62.22%) as a yellow solid. The crude product was used to the next step directly without further purification.

$$LCMS ( ESI + ) \text{:} RT = 0.969 min , m / z 557.2 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Compound 13-ET42365-154

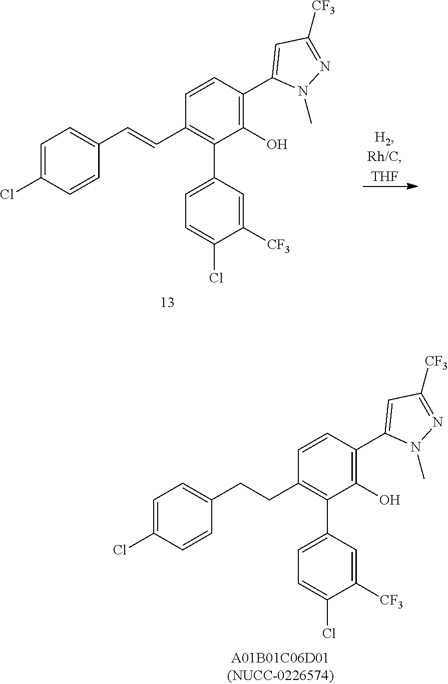 [SMILES: Cn1nc(C(F)(F)F)cc1-c1ccc(/C=C/c2ccc(Cl)cc2)c(-c2ccc(Cl)c(C(F)(F)F)c2)c1O ; "H2,Rh/C,THF" ; Cn1nc(C(F)(F)F)cc1-c1ccc(CCc2ccc(Cl)cc2)c(-c2ccc(Cl)c(C(F)(F)F)c2)c1O]

To a solution of Compound 13 (30 mg, 53.83 μmol, 1 eq) in THF (15 mL) was added Rh/C (30.00 mg) under argon. The suspension was degassed under vacuum and purged with H₂ several times. The mixture was stirred under H₂ balloon at 20° C. for 12 hrs. LCMS showed the starting material was consumed and a new peak with desired product Ms was detected. The reaction was filtered and the filtrate was concentrated to give the crude product. The crude product was purified by Prep-HPLC (column: Waters Xbridge Prep OBD C18 150*40 mm*10 um; mobile phase: [water (10 mM NH₄HCO₃)-ACN]; B %: 70%-95%, 8 min) to give A01B01C06D01 (21.7 mg, yield 69.12%) as a white solid.

¹H NMR (ET42365-172-P1A, 400 MHz, CHLOROFORM-d) δ 2.65-2.79 (m, 4H), 3.86 (s, 3H), 4.82 (s, 1H), 6.61 (s, 1H), 6.83 (d, J=8.38 Hz, 2H), 7.03 (d, J=7.88 Hz, 1H), 7.17-7.21 (m, 2H), 7.22-7.26 (m, 2H), 7.52 (d, J=2.00 Hz, 1H), 7.63 (d, J=8.13 Hz, 1H)

$$LCMS ( ESI + ) \text{:} RT = 3.135 min , m / z 559.1 {( M + H )}^{+} .$$

50_100AB_6min-220-254-ELSD: LC/MS (The gradient was 50% B in 0.40 min and 50-100% B at 0.40-3.00 min, hold on 100% B for 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1000.

### Synthesis of NUCC-0226566 (ET42365-147-1)

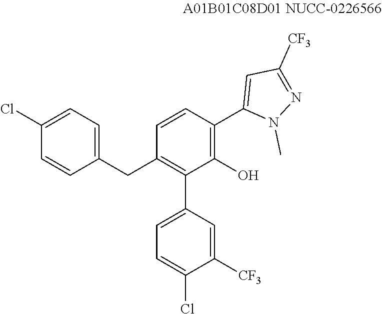 [SMILES: Cn1nc(C(F)(F)F)cc1-c1ccc(Cc2ccc(Cl)cc2)c(-c2ccc(Cl)c(C(F)(F)F)c2)c1O]

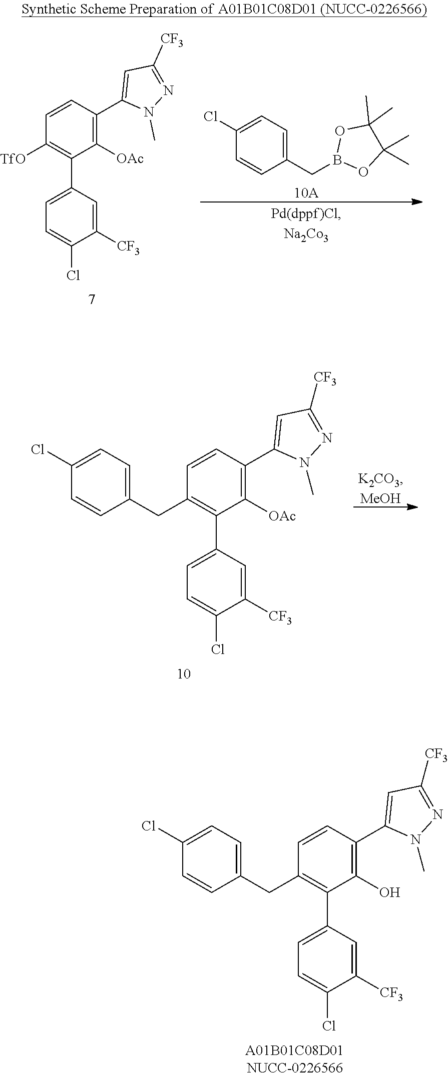

### Chemical Synthesis

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 ml/min.

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (Sum particles). Detection methods are diode array (DAD) and evaporative light scattering (ELSD) detection. MS mode was positive electrospray ionization. MS range was 100-1000.

### Experimentals for Largest Scale Run

### General Procedure for Preparation of Compound 10-ET43365-145

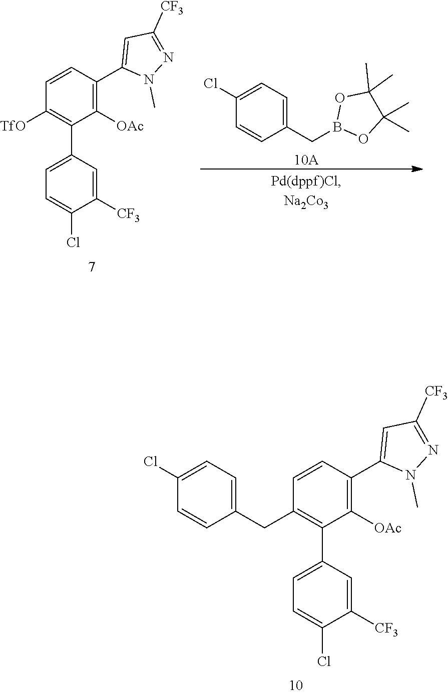

To a solution of Compound 7 (200 mg, 327.42 μmol, 1 eq), 2-[(4-chlorophenyl)methyl]-4,4,5,5-tetramethyl-1,3,2-dioxaborolane (124.03 mg, 491.13 μmol, 1.5 eq) and Na₂CO₃ (69.41 mg, 654.84 μmol, 2 eq) in a mixture solution of dioxane (6 mL) and H₂O (1.5 mL) was added Pd(dppf)Cl₂·CH₂Cl₂ (26.74 mg, 32.74 μmol, 0.1 eq) under nitrogen, the reaction was stirred at 85° C. for 12 hrs. LCMS showed the starting material was consumed and a new peak with desired product Ms was detected. The reaction was filtered and the filtrate was concentrated to give the crude Compound 10 (120 mg, yield 49.92%) as a brown solid. The crude product was used to the next step directly without further purification.

$$LCMS ( ESI + ) \text{:} RT = 0.959 min , m / z 587.2 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

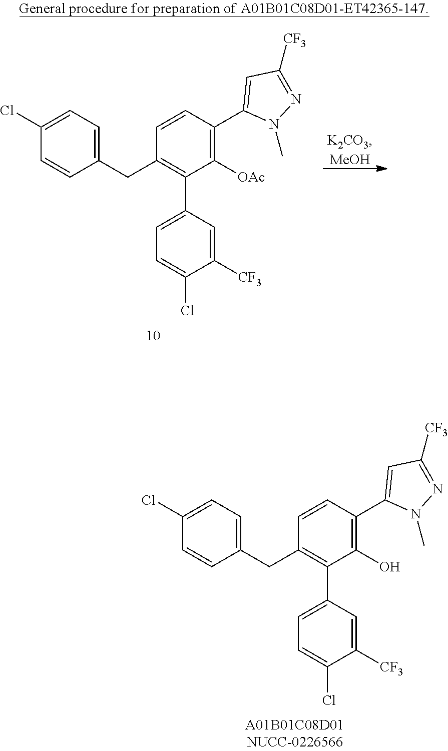

To a solution of Compound 10 (120 mg, ˜163.45 μmol, crude) in MeOH (5 mL) was added K₂CO₃ (45.18 mg, 326.90 μmol, 2 eq), the reaction was stirred at 20° C. for 3 hrs. LCMS showed the starting material was consumed and a new peak with desired product Ms was detected. The reaction mixture was diluted with water (30 mL) and extracted with ethyl acetate (3×30 ml). The organic layer was separated and the combined organic layer was washed with brine (30 mL), dried over Na₂SO₄, filtered and concentrated under reduced pressure to give crude product. The crude product was purified by Prep-HPLC (column: Waters Xbridge Prep OBD C18 150*40 mm*10 um; mobile phase: [water (10 mM NH₄HCO₃)-ACN]; B %: 70%-95%, 8 min) to give A01B01C08D01 (62.8 mg, yield 70.46%) as off-white solid.

¹H NMR (ET42365-147-P1A, 400 MHz, CHLOROFORM-d) δ 3.73 (d, J=3.13 Hz, 2H), 3.87 (s, 3H), 4.86 (br s, 1H), 6.61 (s, 1H), 6.80 (d, J=8.25 Hz, 2H), 6.99 (d, J=7.88 Hz, 1H), 7.19 (d, J=8.38 Hz, 2H), 7.25 (s, 1H), 7.30 (br d, J=1.50 Hz, 1H), 7.41 (d, J=1.50 Hz, 1H), 7.60 (d, J=8.25 Hz, 1H)

$$LCMS ( ESI + ) \text{:} RT = 3.026 min , m / z 545.1 {( M + H )}^{+} .$$

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (Sum particles). Detection methods are diode array (DAD) and evaporative light scattering (ELSD) detection. MS mode was positive electrospray ionization. MS range was 100-1000.

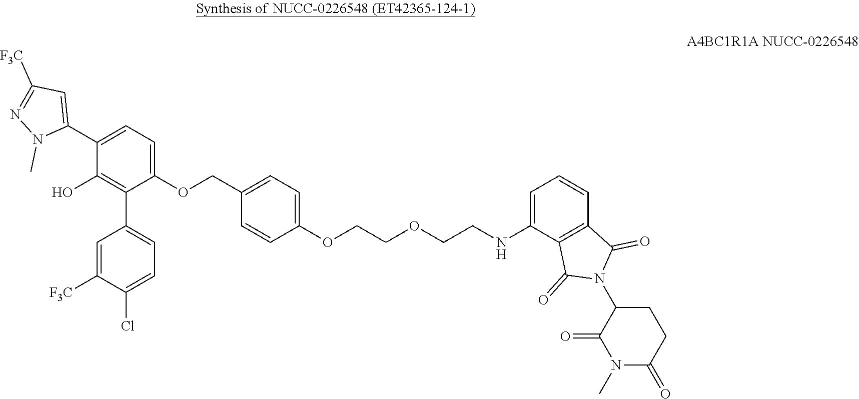 [SMILES: CN1C(=O)CCC(N2C(=O)c3cccc(NCCOCCOc4ccc(COc5ccc(-c6cc(C(F)(F)F)nn6C)c(O)c5-c5ccc(Cl)c(C(F)(F)F)c5)cc4)c3C2=O)C1=O]

### Synthetic Scheme

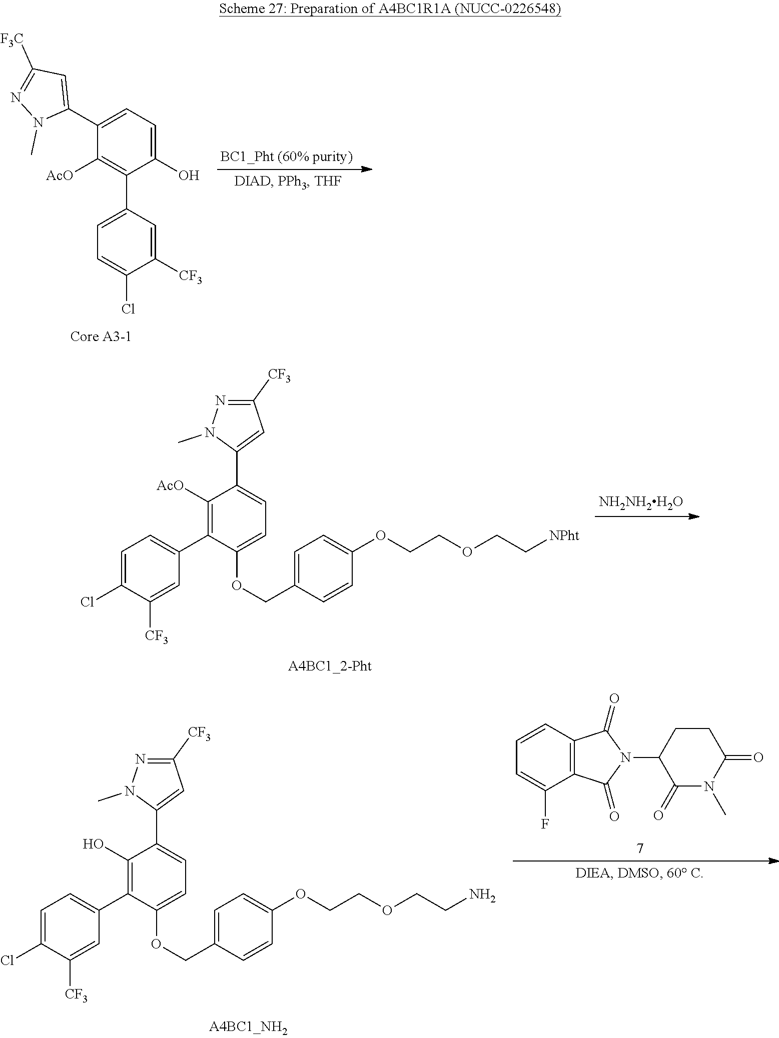

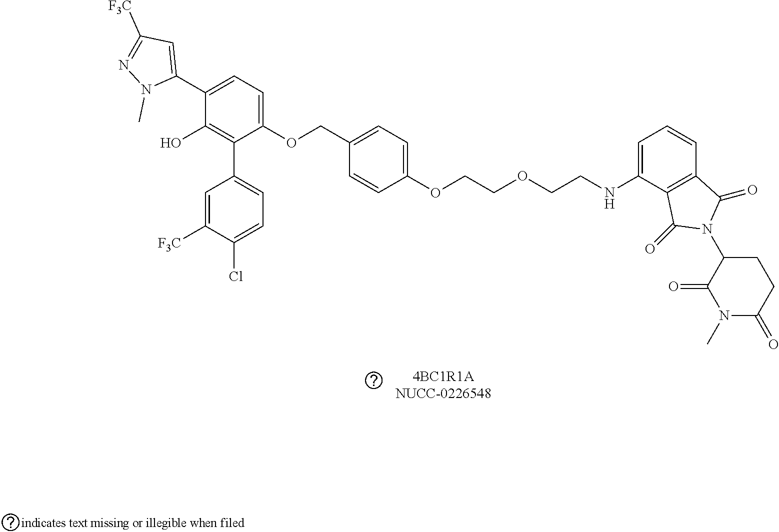 [SMILES: CN1C(=O)CCC(N2C(=O)c3cccc(NCCOCCOc4ccc(COc5ccc(-c6cc(C(F)(F)F)nn6C)c(O)c5-c5ccc(Cl)c(C(F)(F)F)c5)cc4)c3C2=O)C1=O]

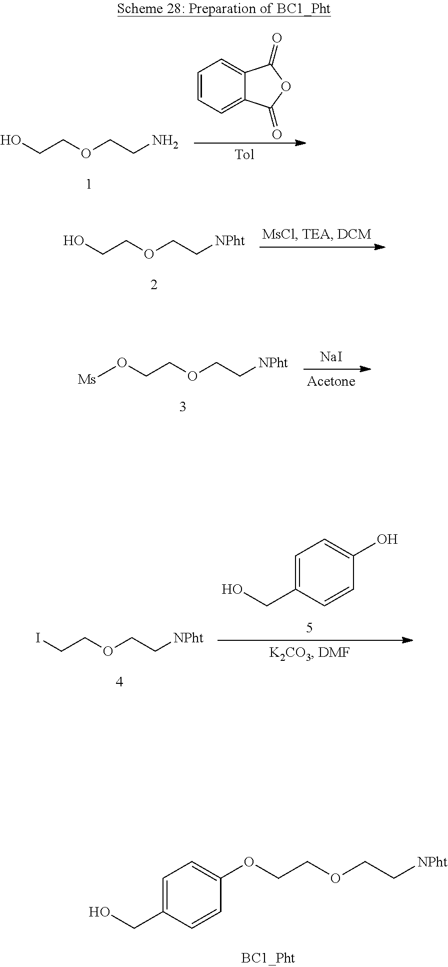

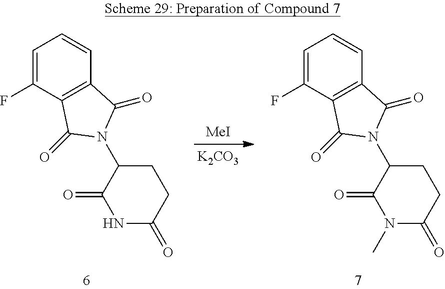

### Chemical Synthesis

5-95AB_2_min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

NEG50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (Sum particles). Detection methods are diode array (DAD) and evaporative light scattering (ELSD) detection. MS mode was negative electrospray ionization. MS range was 100-1000.

### Experimentals for Largest Scale Run

### General Procedure for Preparation of Compound 2-ET43365-97

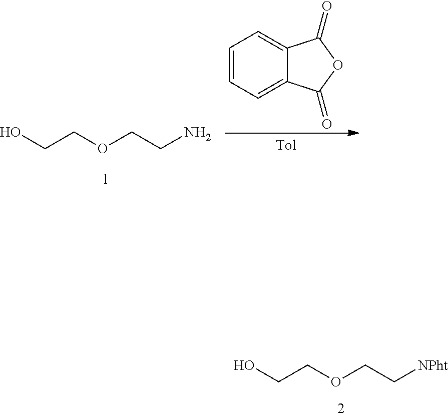

To a solution of Compound 1 (5 g, 47.56 mmol, 4.76 mL, 1 eq) in toluene (60 mL) was added isobenzofuran-1,3-dione (7.04 g, 47.56 mmol, 1 eq). The mixture was stirred at 120° C. for 12 hrs. LCMS showed the starting material was consumed and a new peak with desired product Ms was detected. The reaction was concentrated to give Compound 2 (11 g, yield 88.49%) as a yellow solid. The product was used to the next step directly without further purification.

¹H NMR (ET42365-97-P1A, 400 MHz, CHLOROFORM-d) δ 2.66 (br s, 1H), 3.59-3.63 (m, 2H), 3.67-3.71 (m, 2H), 3.73-3.78 (m, 2H), 3.89-3.95 (m, 2H), 7.70-7.76 (m, 2H), 7.83-7.89 (m, 2H)

### General Procedure for Preparation of Compound 3-ET42365-103

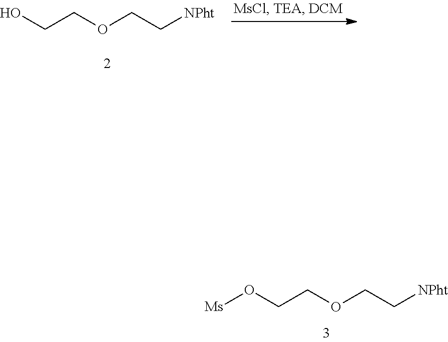

To a solution of Compound 2 (3 g, 12.75 mmol, 1 eq) and Et₃N (3.23 g, 31.88 mmol, 4.44 mL, 2.5 eq) in DCM (60 mL) was added MsCl (2.19 g, 19.13 mmol, 1.48 mL, 1.5 eq) dropwise at 0° C., the reaction was stirred at 20° C. for 12 hrs. LCMS showed the starting material was consumed and a new peak with desired product Ms was detected. The reaction mixture was diluted with water (100 mL) and extracted with DCM (2×100) mL). The organic layer was separated and the combined organic layer was washed with brine (200 mL), dried over Na₂SO₄, filtered and concentrated under reduced pressure to crude product. The crude product was purified by column chromatography on silica gel (petroleum ether/ethyl acetate=20/1 to 2/1) to give Compound 3 (3.7 g, yield 87.97%) as a yellow solid.

¹H NMR (ET42365-103-P1A, 400 MHz, CHLOROFORM-d) δ 3.00 (s, 3H), 3.71-3.82 (m, 4H), 3.88-3.95 (m, 2H), 4.28-4.36 (m, 2H), 7.70-7.78 (m, 2H), 7.82-7.91 (m, 2H)

### General Procedure for Preparation of Compound 4-ET43587-64

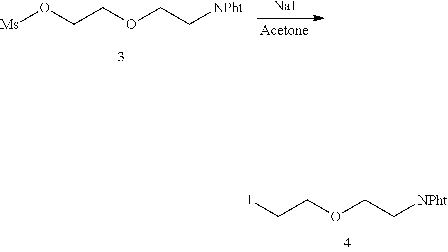

To a solution of Compound 3 (3 g, 9.57 mmol, 8.47 mL, 1 eq) in acetone (45 mL) was added NaI (7.18 g, 47.87 mmol, 5 eq). The mixture was stirred at 65° C. for 12 hrs. TLC (petroleum ether/ethyl acetate=1/1) showed the starting material was consumed and a new spot was generated. The reaction mixture was diluted with water (30 mL) and extracted with ethyl acetate (3×20 mL). The organic layer was separated and the combined organic layer was washed with brine (20 mL), dried over Na₂SO₄, filtered and concentrated under reduced pressure to give a residue. The crude product was purified by chromatography on silica gel (eluted with petroleum ether/ethyl acetate=50/1 to 3/1) to give Compound 4 (3 g, yield 81.71%) was obtained as a red oil.

$$LCMS ( ESI + ) \text{:} RT = 0.734 min , m / z 346. {( M + H )}^{+} .$$

5-95AB_2_min: LC/MS (The column used for chromatography was a Kinetex EVO C18 2.1*30 mm, Sum. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 ml/min.

### General Procedure for Preparation of Compound BC1_Pht-ET42365-118

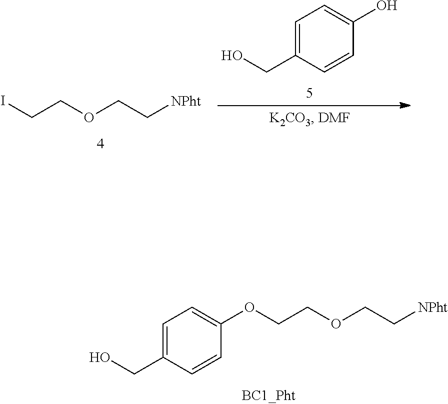

To a solution of Compound 5 (431.62 mg, 3.48 mmol, 1.2 eq) and K₂CO₃ (600.67 mg, 4.35 mmol, 1.5 eq) in CH₃CN (15 mL) was added Compound 4 (1 g, 2.90 mmol, 1 eq), the reaction was stirred at 90° C. for 12 hrs. LCMS showed about 10% of the starting material is remaining and a new peak with desired product Ms was detected. The reaction mixture was diluted with water (60 mL) and extracted with ethyl acetate (3×50 mL). The organic layer was separated and the combined organic layer was washed with brine (50 mL), dried over Na₂SO₄, filtered and concentrated under reduced pressure to crude product. The crude product was purified by column chromatography on silica gel (eluted with petroleum ether/ethyl acetate=50/1 to 10/1) to give BC1_Pht (0.6 g, yield 36.4%) as a colorless oil.

$$LCMS ( ESI + ) \text{:} RT = 0.656 min , m / z 324.2 {( M + H )}^{+} .$$

5-95AB_2_min: LC/MS (The column used for chromatography was a Kinetex EVO C18 2.1*30 mm, Sum. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

### General Procedure for Preparation of A4BC1_2-Pht-ET42365-119

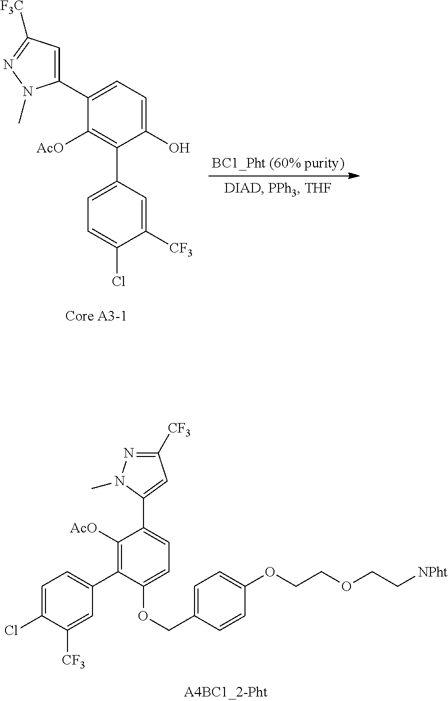

To a solution of Core A3-1 (400 mg, 835.47 μmol, 1 eq), BC1_Pht (342.23 mg, 1.00 mmol, 1.2 eq) and PPh₃ (876.54 mg, 3.34 mmol, 4 eq) in THF (10 mL) was added DIAD (675.76 mg, 3.34 mmol, 649.77 μL, 4 eq) dropwise at 0° C. the reaction was stirred at 25° C.′ for 12 hrs. LCMS showed the starting material was consumed and a new peak with desired product Ms was detected. The reaction mixture was diluted with water (60 mL) and extracted with ethyl acetate (3×50 mL). The organic layer was separated and the combined organic layer was washed with brine (50 mL), dried over Na₂SO₄, filtered and concentrated under reduced pressure to give crude product. The crude product was purified by column chromatography on silica gel (eluted with petroleum ether/ethyl acetate=50/1 to 3/1) to give A4BC1_2-Pht (0.6 g, yield 71.63%) as a colorless oil.

$$LCMS ( ESI + ) \text{:} RT = 0.951 min , m / z 824.3 {( M + H )}^{+} .$$

5-95AB_2_min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Compound 7-ET42365-114

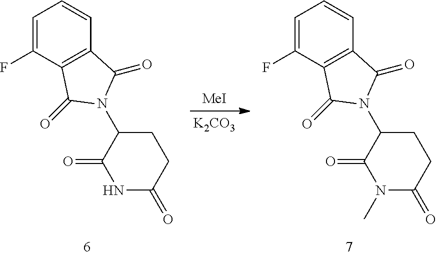

To a solution of Compound 6 (1 g, 3.62 mmol, 1 eq) in DMF (15 mL) were added K₂CO₃ (750.52 mg, 5.43 mmol, 1.5 eq) and iodomethane (1.03 g, 7.24 mmol, 450.76 μL, 2 eq) at 0° C., the reaction was stirred at 20° C. for 12 hrs. LCMS showed the starting material was consumed and a new peak with desired product Ms was detected. The reaction was diluted with water (60 mL) and stirred for 1 hour. The solid was filtered and dried under high vacuum to obtain Compound 7 (0.8 g. yield 76.13%) as a pink solid. The product was used to the next step directly without further purification.

¹H NMR (ET42365-114-P1A, 400 MHz, CHLOROFORM-d) δ 2.05-2.21 (m, 1H), 2.71-2.90 (m, 2H), 2.93-3.08 (m, 1H), 3.22 (s, 3H), 4.99 (br dd, J=12.67, 5.40 Hz, 1H), 7.43 (t, J=8.41 Hz, 1H), 7.69-7.74 (m, 1H), 7.74-7.82 (m, 1H)

### General Procedure for Preparation of A4BC1 NH₂-ET43259-123

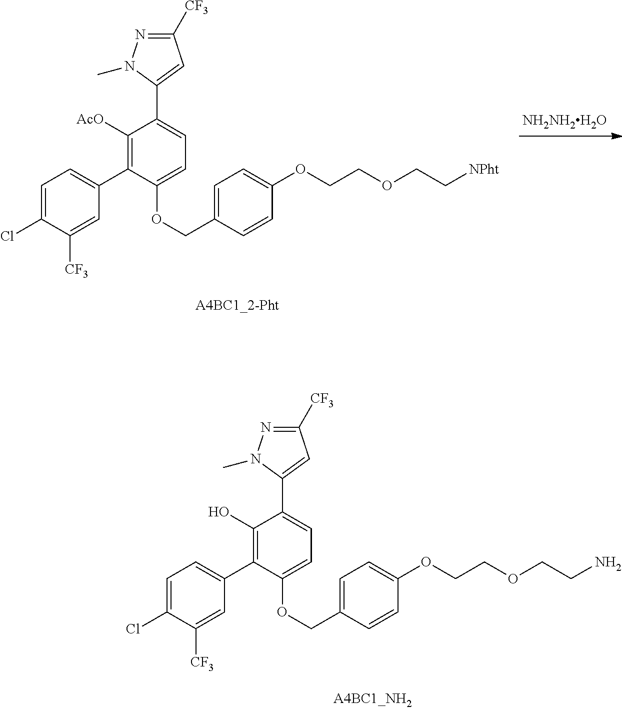

To a solution of A4BC1_2-Pht (600 mg, 598.42 μmol, 80% purity, 1 eq) in EtOH (10 mL) was added NH₂NH₂·H₂O (299.57 mg, 5.98 mmol, 290.84 μL, 10 eq) and the reaction was stirred at 25° C. for 1 hour. LCMS showed the starting material was consumed and a new peak with desired product Ms was detected. The reaction mixture was diluted with water (60 mL) and extracted with ethyl acetate (3×50 mL). The organic layer was separated and the combined organic layer was washed with brine (50 mL), dried over Na₂SO₄, filtered and concentrated under reduced pressure to give crude A4BC1_NH2 (300 mg, yield 71.62%) as a yellow solid. The crude product was used to the next step directly without further purification.

$$LCMS ( ESI + ) \text{:} RT = 0.736 min , m / z 630.3 {( M + H )}^{+} .$$

S-95AB_2_min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

### General Procedure for Preparation of A4BC1R1A-ET42365-124

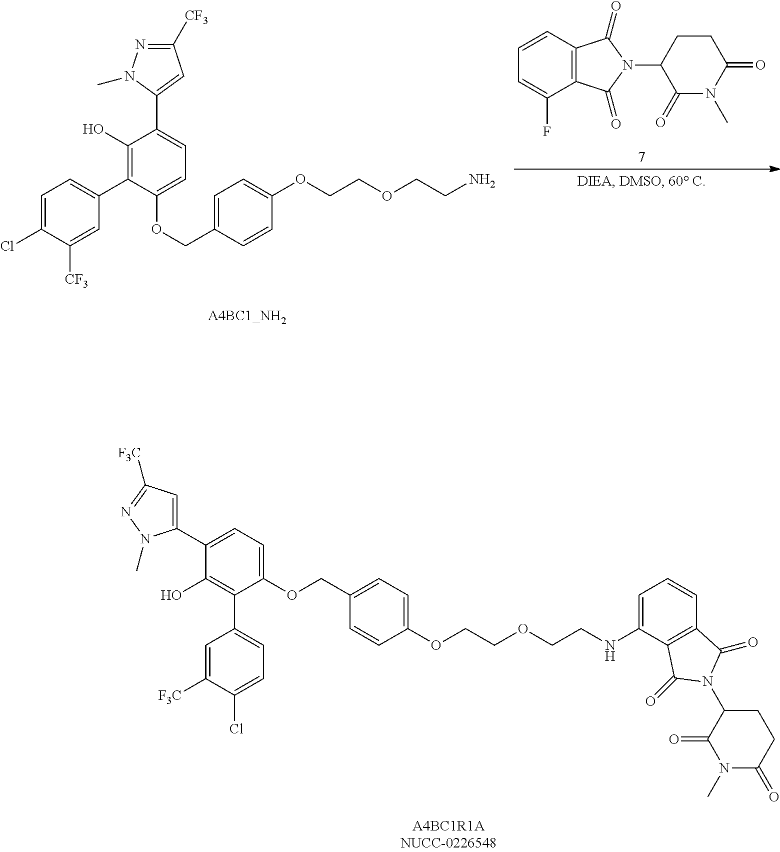

To a solution of A4BC1_NH2 (300 mg, 401.78 μmol, 90% purity, 1 eq) in DMSO (6 mL) were added DIEA (155.78 mg, 1.21 mmol, 209.95 μL, 3 eq) and Compound 7 (233.23 mg, 803.56 μmol, 2 eq), the reaction was stirred at 60° C. for 12 hrs. LCMS showed the starting material was consumed and a new peak with desired product Ms was detected. The reaction was purified by Prep-HPLC (column: Waters Xbridge Prep OBD C18 150*40 mm*10 um; mobile phase: [water (10 mM NH₄HCO₃)-ACN]; B %: 60%-80%, 8 min) and lyophilized to give A4BC1R1A (108.9 mg, yield 30.08%) as a yellow solid.

¹H NMR (ET42365-124-P1A, 400 MHz, CHLOROFORM-d) δ 2.04-2.13 (m, 1H), 2.68-2.83 (m, 2H), 2.90-3.04 (m, 1H), 3.21 (s, 3H), 3.51 (t, J=5.38 Hz, 2H), 3.80 (t, J=5.44 Hz, 2H), 3.83-3.90 (m, 5H), 4.10-4.18 (m, 2H), 4.86-4.95 (m, 1H), 5.02 (s, 2H), 5.11 (s, 1H), 6.57 (s, 1H), 6.78 (d, J=8.63 Hz, 1H), 6.88 (d, J=8.76 Hz, 2H), 6.93 (d, J=8.50 Hz, 1H), 7.09 (d, J=7.13 Hz, 1H), 7.15 (d, J=8.63 Hz, 2H), 7.21 (d, J=8.51 Hz, 1H), 7.47 (dd, J=8.51, 7.25 Hz, 1H), 7.51-7.55 (m, 1H), 7.57-7.63 (m, 1H), 7.79 (d, J=1.75 Hz, 1H)

$$LCMS ( ESI + ) \text{:} RT = 2.569 min , m / z 898.2 {( M + H )}^{+} .$$

NEG50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (Sum particles). Detection methods are diode array (DAD) and evaporative light scattering (ELSD) detection. MS mode was negative electrospray ionization. MS range was 100-1000.

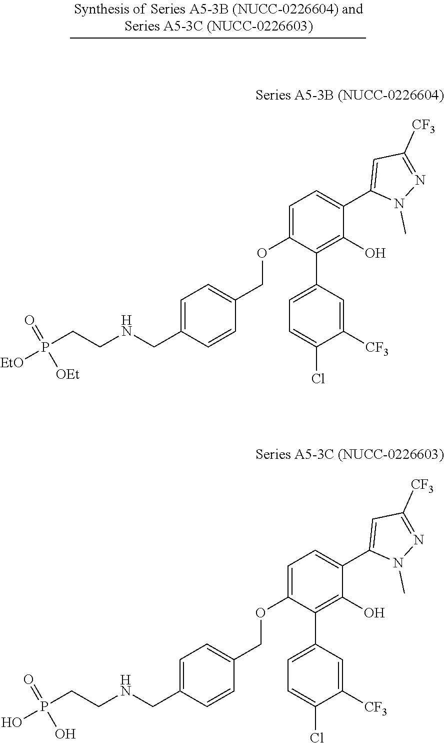 [SMILES: CCOP(=O)(CCNCc1ccc(COc2ccc(-c3cc(C(F)(F)F)nn3C)c(O)c2-c2ccc(Cl)c(C(F)(F)F)c2)cc1)OCC ; Cn1nc(C(F)(F)F)cc1-c1ccc(OCc2ccc(CNCCP(=O)(O)O)cc2)c(-c2ccc(Cl)c(C(F)(F)F)c2)c1O]

### Synthetic Scheme

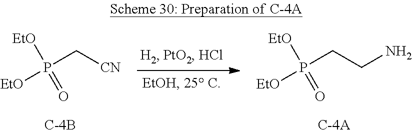

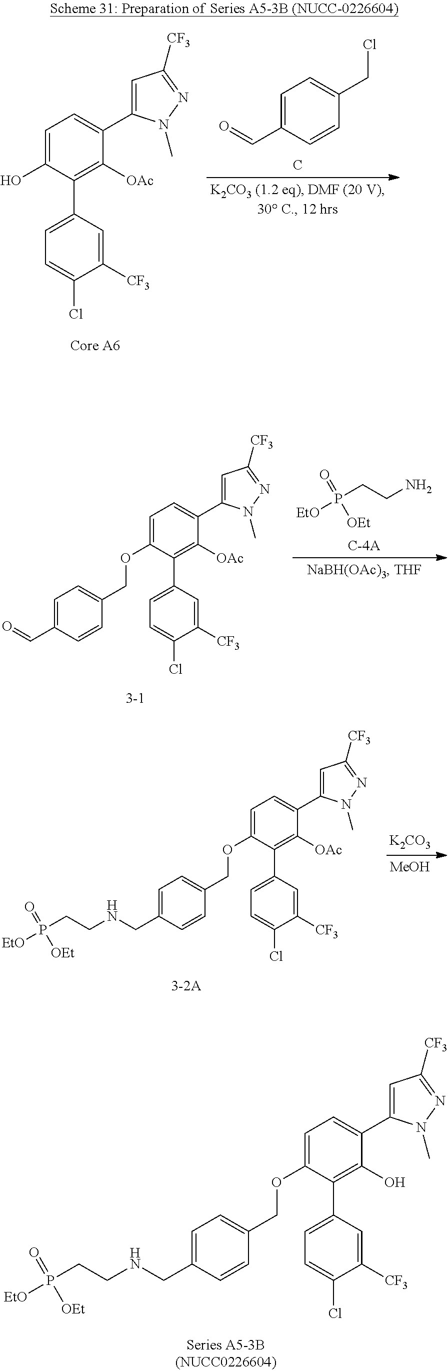

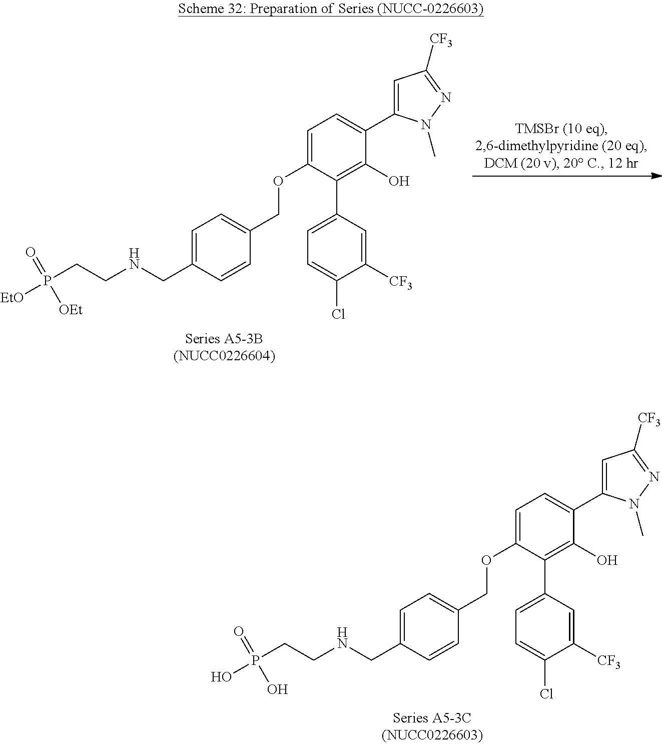 [SMILES: CCOP(=O)(CCNCc1ccc(COc2ccc(-c3cc(C(F)(F)F)nn3C)c(O)c2-c2ccc(Cl)c(C(F)(F)F)c2)cc1)OCC ; Cn1nc(C(F)(F)F)cc1-c1ccc(OCc2ccc(CNCCP(=O)(O)O)cc2)c(-c2ccc(Cl)c(C(F)(F)F)c2)c1O]

### LCMS Methods:

### Method 1: 5_95AB_6min-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

### Method 2: 5_95CD_6min-220-254-ELSD

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge Shield RP18 2.1*50 mm column (5 um particles). Detection methods are diode array (DAD) and evaporative light scattering (ELSD) detection as well as positive electrospray ionization. MS range was 100-1000.

### Experimentals for Largest Scale Run

### General Procedure for Preparation of C-4A-ET43318-125

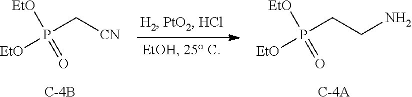

To a mixture of C-4B (2 g, 11.29 mmol, 1.82 mL), HCl (12 M, 940.89 μL, 1 eq) in EtOH (30 mL) was added PtO₂ (256.38 mg, 1.13 mmol) at 20° C. The mixture was stirred for 12 hrs under 15 psi pressure hydrogen. TLC (ethyl acetate/MeOH=5/1) showed the starting material was consumed and new spot generated. Product with desired Ms was detected by LCMS. The mixture was filtered through celite and the filtrate was concentrated under reduced pressure to give C-4A (2.2 g, 71.63% yield) as a yellow solid which was used directly for next step without further purification.

¹H NMR (400 MHz, CHLOROFORM-d) δ 1.34 (t, J=7.07 Hz, 6H), 2.41 (dt, J=18.51, 7.75 Hz, 2H), 3.15-3.38 (m, 2H), 4.03-4.26 (m, 4H), 7.70-8.72 (m, 2H)

### General Procedure for Preparation of Compound 3-1-ET43318-157

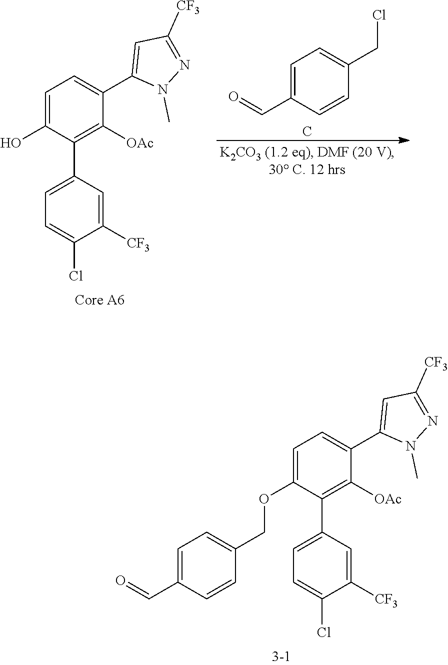

To a solution of C (161.45 mg, 1.04 mmol), Core A6 (500 mg, 1.04 mmol) in DMF (5 mL) was added Cs₂CO₃ (340.27 mg, 1.04 mmol) at 20° C. The mixture was stirred for 12 hrs.

LCMS showed 12.9% the starting material was remaining and 45.5% peak (Rt=0.893 min) with desired Ms was detected. The reaction mixture was poured into water (100 mL) and extracted with ethyl acetate (30 mL×3). The organic layers were combined, washed with brine (2×10 mL), dried over Na₂SO₄, filtered and concentrated under reduced pressure to give a residue. The residue was purified by column chromatography on silica gel (eluting with petroleum ether/ethyl acetate=20/1 to 3/1) to give 3-1 (250 mg, 24.06% yield) as a white solid.

¹H NMR (400 MHz, CHLOROFORM-d) δ 1.79 (s, 3H), 3.82 (s, 3H), 5.22 (s, 2H), 6.51 (s, 1H), 6.79 (d, J=8.00 Hz, 1H), 7.06 (d, J=8.63 Hz, 1H), 7.32-7.36 (m, 1H), 7.38-7.43 (m, 3H), 7.49 (dd, J=8.25, 1.75 Hz, 1H), 7.57 (br d, J=8.50 Hz, 1H), 7.68 (d, J=8.13 Hz, 1H), 7.76 (d, J=1.75 Hz, 1H), 10.02 (s, 1H)

### General Procedure for Preparation of Compound 3-2A-ET43318-165

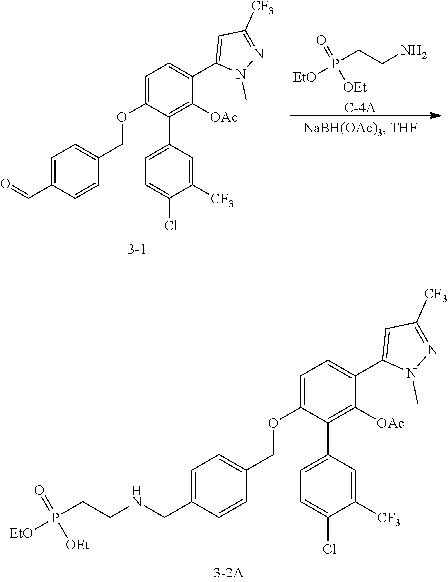

To a solution of C-4A (47.07 mg, 216.27 μmol) of in THF (2 mL) was added TEA (54.71 mg, 540.67 μmol) at 20° C. The mixture was stirred for 0.5 hr at 20° C., then 3-1 (100 mg, 180.22 μmol) was added into. After stirring for 2 hrs, AcOH (32.47 mg, $40.67 μmol) and NaBH(OAc)₃ (76.39 mg, 360.45 μmol) was added. The mixture was stirred for another 2.5 hrs. LCMS showed the starting material was consumed and 52.8% peak (Rt=0.750 min) with desired Ms was detected. TLC (petroleum ether/ethyl acetate=2:1) showed new spot generated. The reaction mixture was poured into saturated NaHCO₃ (60 mL) and extracted with ethyl acetate (60 mL×3). The organic layers were combined, washed with brine (2×10 mL), dried over Na₂SO₄, filtered and concentrated under reduced pressure to give a residue. The residue was purified by Prep-TLC (ethyl acetate/MeOH=5/1) to give 3-2A (80 mg, 42.52% yield) as a colorless oil.

¹H NMR (400 MHz, CHLOROFORM-d) δ 1.31 (t, J=7.07 Hz, 6H), 1.61 (br s, 4H), 1.78 (s, 3H), 2.00 (dt, J=18.17, 7.18 Hz, 2H), 2.87-2.99 (m, 2H), 3.75-3.84 (m, 5H), 4.05-4.16 (m, 4H), 5.12 (s, 2H), 6.50 (s, 1H), 7.07 (d, J=8.63 Hz, 1H), 7.20 (d, J=8.00 Hz, 2H), 7.28-7.35 (m, 3H), 7.44-7.50 (m, 1H), 7.51-7.57 (m, 1H), 7.73 (d, J=1.75 Hz, 1H)

### General Procedure for Preparation of Compound Series A5-3B-ET43318-168

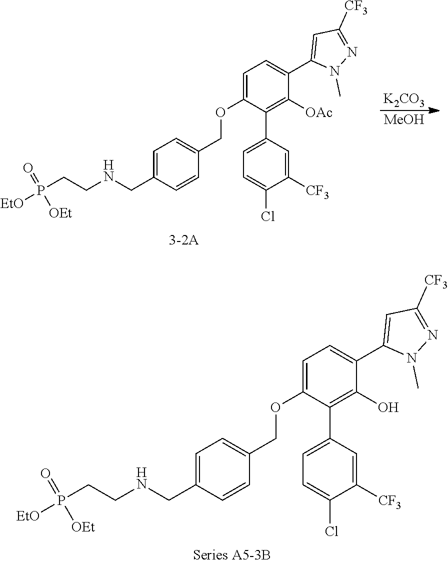

To a solution of 3-2A (80 mg, 104.98 μmol) in MeOH (1 mL) was added K₂CO₃ (43.53 mg, 314.93 μmol) at 20° C. The mixture was stirred for 12 hrs. LCMS showed the starting material was consumed and 86% peak (Rt=0.737 min) with desired Ms was detected. The mixture was filtered to give a filtrate. The filtrate was purified by prep-HPLC to give Series A5-3B (30.6 mg, 40.48% yield) as an off-white solid.

Method of prep-HPLC:
Instrument: Gilson 281 semi-preparative HPLC system
Mobile phase: A: 10 mM NH₄HCO₃ in H2O; B; Acetonitrile
Column: Waters Xbridge BEH C18 100*30 mm*10 um
Flow rate: 25 mL/min
Monitor wavelength: 220&254 nm

| Time | B % |
|---|---|
| 0.0 | 50 |
| 10.0 | 80 |
| 10.1 | 80 |
| 10.2 | 100 |
| 13.2 | 100 |
| 13.3 | 50 |
| 14.5 | 50 |

¹H NMR (400 MHz, CHLOROFORM-d) δ 1.30 (t, J=7.03 Hz, 6H), 1.95 (t, J=7.15 Hz, 1H), 1.99 (t, J=7.15 Hz, 1H), 2.89 (dt, J=15.83, 7.18 Hz, 2H), 3.76 (s, 2H), 3.84 (s, 3H), 4.01-4.13 (m, 4H), 5.08 (s, 2H), 6.56 (s, 1H), 6.75 (d, J=8.68 Hz, 1H), 7.19 (t, J=7.83 Hz, 3H), 7.27-7.30 (m, 2H), 7.51-7.56 (m, 1H), 7.57-7.63 (m, 1H), 7.80 (d, J=1.83 Hz, 1H)

$$LCMS ( ESI + ) \text{:} RT = 2.476 min , m / z 720.1 {( M + H )}^{+} .$$

### 5_95AB_6min-220-254-ELSD

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

### General Procedure for Preparation of Series A5-3C (NUCC-0226603)-ET43318-197

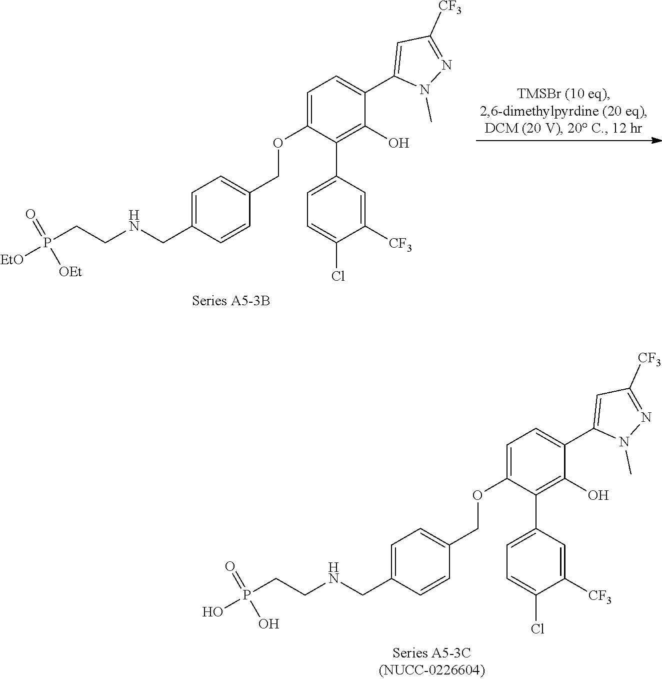 [SMILES: CCOP(=O)(CCNCc1ccc(COc2ccc(-c3cc(C(F)(F)F)nn3C)c(O)c2-c2ccc(Cl)c(C(F)(F)F)c2)cc1)OCC ; Cn1nc(C(F)(F)F)cc1-c1ccc(OCc2ccc(CNCCP(=O)(O)O)cc2)c(-c2ccc(Cl)c(C(F)(F)F)c2)c1O]

To a solution of Series A5-3B (25 mg, 34.72 μmol), 2,6-dimethylpyridine (148.82 mg, 1.39 mmol) in DCM (1 mL) was added TMSBr (106.31 mg, 694.41 μmol) dropwise at 0° C. The mixture was warmed up to 20° C. and stirred for 12 hrs. LCMS showed the starting material was consumed and 60.3% product (Rt=0.788 min) with desired Ms was detected. The mixture was filtered to give a filtrate. The filtrate was purified by prep-HPLC to give Series A5-3C (7.5 mg, 32.34% yield) as a white solid.

Method of prep-HPLC:
Instrument: Gilson 281 semi-preparative HPLC system
Mobile phase: A: 10 mM NH₄HCO₃ in H₂O; B: Acetonitrile
Column: Waters Xbridge BEH C18 100*30 mm*10 um
Flow rate: 25 mL/min
Monitor wavelength: 220&254 nm

| Time | B % |
|---|---|
| 0.0 | 25 |
| 10.0 | 50 |
| 10.1 | 50 |
| 10.2 | 100 |
| 13.2 | 100 |
| 13.3 | 25 |
| 14.5 | 25 |

¹H NMR (400 MHz, METHANOL-d₄) δ 1.79-2.02 (m, 2H), 3.26 (br dd, J=14.57, 7.57 Hz, 2H), 3.79 (s, 3H), 4.18 (s, 2H), 5.14 (s, 2H), 6.58 (s, 1H), 6.86 (d, J=8.75 Hz, 1H), 7.25 (d, J=8.63 Hz, 1H), 7.34 (d, J=8.13 Hz, 2H), 7.44 (br d, J=7.88 Hz, 2H), 7.58-7.67 (m, 2H), 7.78 (d, J=1.50 Hz, 1H)

$$LCMS ( ESI + ) : RT = 2.741 min , m / z 664.2 {( M + H )}^{+} .$$

### 5_95CD_6min-220-254-ELSD

5_95CD_6min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was Acetonitrile. The column used for chromatography was a Xbridge Shield RP18 2.1*50 mm column (Sum particles). Detection methods are diode array (DAD) and evaporative light scattering (ELSD) detection as well as positive electrospray ionization. MS range was 100-1000.

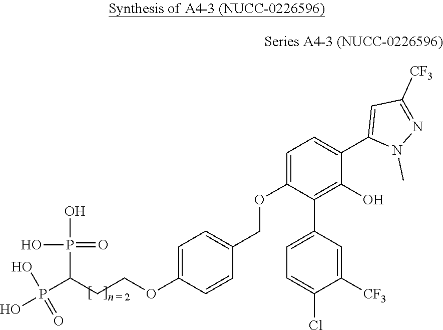

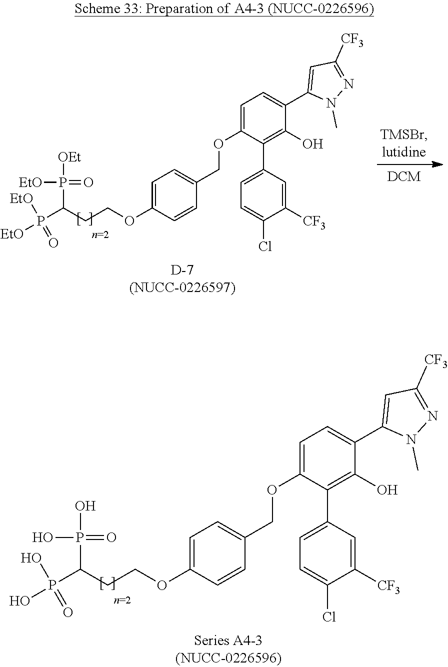 [SMILES: CCOP(=O)(OCC)C(CCOc1ccc(COc2ccc(-c3cc(C(F)(F)F)nn3C)c(O)c2-c2ccc(Cl)c(C(F)(F)F)c2)cc1)P(=O)(OCC)OCC ; Cn1nc(C(F)(F)F)cc1-c1ccc(OCc2ccc(OCCC(P(=O)(O)O)P(=O)(O)O)cc2)c(-c2ccc(Cl)c(C(F)(F)F)c2)c1O]

### Chemical Synthesis

### LCMS Methods:

### Method 1: 5_95CD_6min-220-254-ELSD

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge Shield RP18 2.1*50 mm column (5 um particles). Detection methods are diode array (DAD) and evaporative light scattering (ELSD) detection as well as positive electrospray ionization. MS range was 100-1000.

### Experimentals for Largest Scale Run

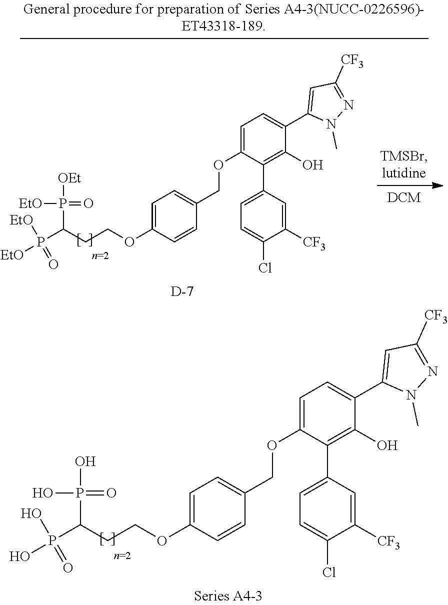

To a solution of D-7 (25 mg, 28.70 μmol), 2,6-dimethylpyridine (61.50 mg, 0.574 mmol) in DCM (0.5 mL) was added TMSBr (43.93 mg, 0.287 mmol) dropwise at 0° C. The mixture was warmed up to 20° C. and stirred for 12 hrs. LCMS showed the starting material was consumed and 18.3% product with desired Ms was detected. The mixture was filtered to give a filtrate. The filtrate was purified by prep-HPLC to give Series A4-3 (3.4 mg, 15.36% yield) as a white solid.

Method of prep-HPLC:
Instrument: Gilson 281 semi-preparative HPLC system
Mobile phase: A: 10 mM NH₄HCO₃ in H₂O; B: Acetonitrile
Column: Waters Xbridge BEH C18 100*30 mm*10 um
Flow rate: 25 mL/min
Monitor wavelength: 220&254 nm

| Time | B % |
|---|---|
| 0.0 | 5 |
| 10.0 | 35 |
| 10.1 | 35 |
| 10.2 | 100 |
| 13.2 | 100 |
| 13.3 | 5 |
| 14.5 | 5 |

¹H NMR (400 MHz, METHANOL-d₄) δ 2.10 (br s, 5H), 3.79 (s, 3H), 3.99 (br t, J=5.13 Hz, 2H), 5.02 (s, 2H), 6.59 (s, 1H), 6.86 (dd, J=8.57, 5.94 Hz, 3H), 7.15 (d, J=8.50 Hz, 2H), 7.23 (d, J=8.50 Hz, 1H), 7.54-7.59 (m, 1H), 7.60-7.66 (m, 1H), 7.74 (d, J=1.38 Hz, 1H)

$$LCMS ( ESI + ) : RT = 2.575 min , m / z 759.1 {( M + H )}^{+} .$$

### 5_95CD_6min-220-254-ELSD

5_95CD_6min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H2O+10 mM NH₄HCO₃, mobile phase B was Acetonitrile. The column used for chromatography was a Xbridge Shield RP18 2.1*50 mm column (Sum particles). Detection methods are diode array (DAD) and evaporative light scattering (ELSD) detection as well as positive electrospray ionization. MS range was 100-1000.

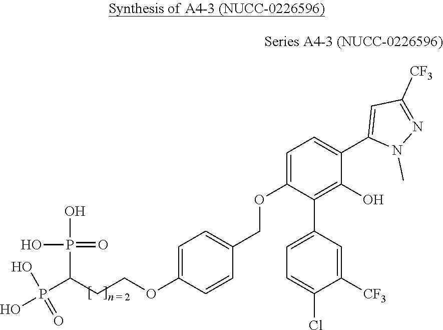

### Synthetic Scheme

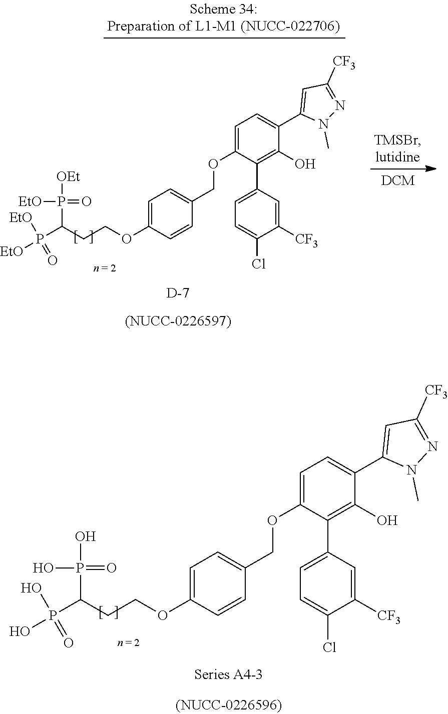

### LCMS Methods:

### Method 1: 5_95CD_6min-220-254-ELSD

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge Shield RP18 2.1*50 mm column (5 um particles). Detection methods are diode array (DAD) and evaporative light scattering (ELSD) detection as well as positive electrospray ionization. MS range was 100-1000.

### Experimentals for Largest Scale Run

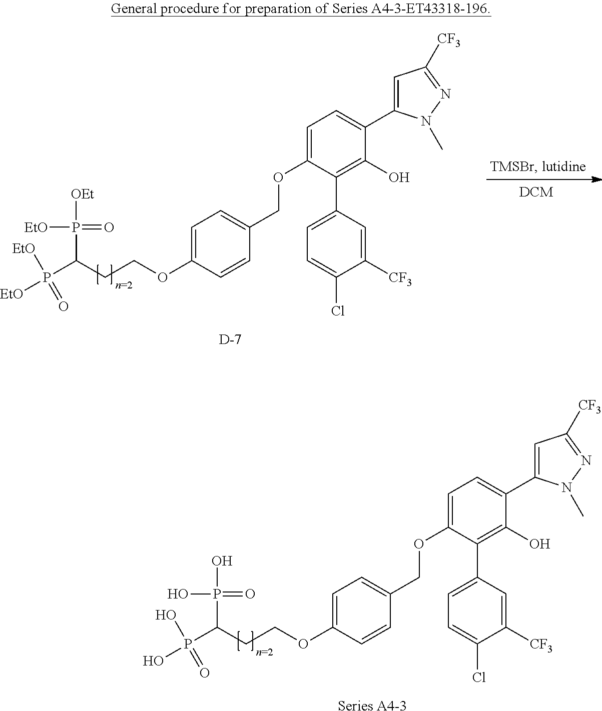

To a solution of D-7 (60 mg, 68.88 μmol), 2,6-dimethylpyridine (295.21 mg, 2.76 mmol) in DCM (2 mL) was added TMSBr (210.89 mg, 1.38 mmol) dropwise at 0° C. The mixture was warmed up to 20° C. and stirred for 2 hrs. LCMS showed the starting material was consumed and 18.6% product with desired Ms was detected. The mixture was filtered to give a filtrate. The filtrate was purified by prep-HPLC to give Series A4-3 (11.2 mg, 21.43% yield) as a white solid.

Method of prep-HPLC:
Instrument: Gilson 281 semi-preparative HPLC system
Mobile phase: A: 10 mM NH₄HCO₃ in H₂O; B: Acetonitrile
Column: Waters Xbridge BEH C18 100*30 mm*10 um
Flow rate: 25 mL/min
Monitor wavelength: 220&254 nm

| Time | B % |
|---|---|
| 0.0 | 15 |
| 10.0 | 45 |
| 10.1 | 45 |
| 10.2 | 100 |
| 13.2 | 100 |
| 13.3 | 15 |
| 14.5 | 15 |

¹H NMR (400 MHz, METHANOL-d₄) δ 2.11 (br s, 5H), 3.79 (s, 3H), 3.93-4.03 (m, 2H), 5.02 (s, 2H), 6.58 (s, 1H), 6.85 (dd, J=8.57, 6.44 Hz, 3H), 7.14 (d, J=8.63 Hz, 2H), 7.23 (d, J=8.63 Hz, 1H), 7.52-7.59 (m, 1H), 7.60-7.65 (m, 1H), 7.74 (d, J=1.75 Hz, 1H)

LCMS (ESI+): RT=2.571 min, m/z 759.1 (M+H)⁺.

### 5_95CD_6min-220-254-ELSD

5_95CD_6min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H2O+10 mM NH4HCO3, mobile phase B was Acetonitrile. The column used for chromatography was a Xbridge Shield RP18 2.1*50 mm column (5 um particles). Detection methods are diode array (DAD) and evaporative light scattering (ELSD) detection as well as positive electrospray ionization. MS range was 100-1000.

### Synthesis of D-7 (NUCC-0226597)

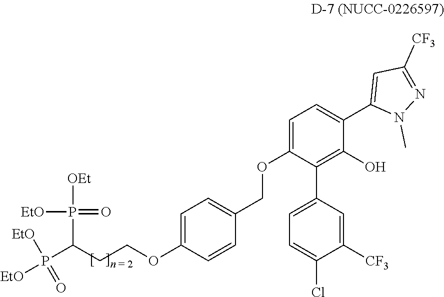

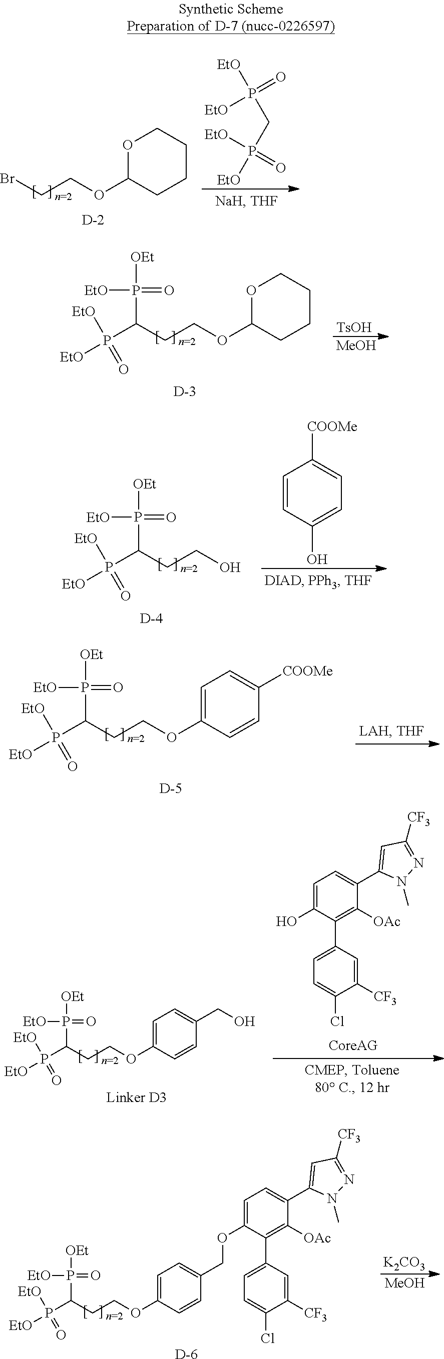

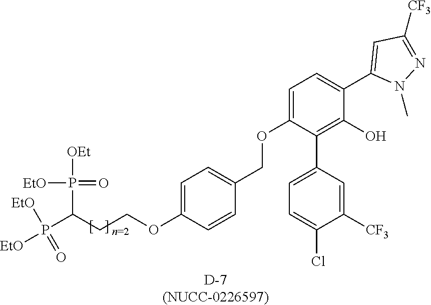

### LCMS Methods:

### Method 1: 5_95AB_6min-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

### Experimentals for Largest Scale Run

### General Procedure for Preparation of Compound D-3-ET43318-118

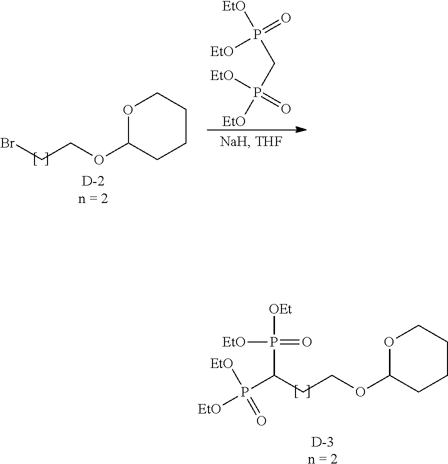

To a solution of 1-[diethoxyphosphorylmethyl(ethoxy)phosphoryl]oxyethane (10 g, 34.70 mmol) in THF (100 mL) was added NaH (1.53 g, 38.17 mmol) portionwise at 0° C. under nitrogen. After stirring for 30 min, D-2 (9.29 g, 41.64 mmol) was added into. The mixture was heated to 70° C. and stirred for 12 hrs. TLC (ethyl acetate/MeOH=5/1. Rf=0.55) showed the starting material was consumed and new spot generated. The reaction mixture was poured into NH₄Cl (300 mL) and extracted with ethyl acetate (3×100 ml). The organic layers were combined, washed with brine (2×50 ml), dried over Na₂SO₄, filtered and concentrated under reduced pressure to give D-3 (12 g, crude) as a yellow oil which was used directly for next step without further purification.

¹H NMR (400 MHz, CHLOROFORM-d) δ 1.32-1.37 (m, 12H), 1.55-1.61 (m, 4H), 1.73 (br dd, J=6.50, 3.00 Hz, 1H), 1.81-1.92 (m, 3H), 1.96-2.09 (m, 2H), 2.26-2.48 (m, 1H), 3.52-3.55 (m, 2H), 3.85-3.90 (m, 2H), 4.15-4.23 (m, 8H), 4.58-4.61 (m, 1H)

### General Procedure for Preparation of Compound D-4-ET43318-137

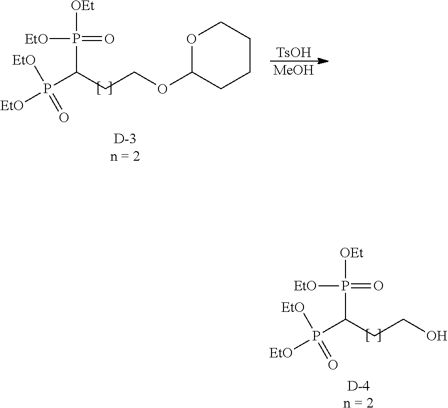

To a solution of D-3 (5 g, 11.62 mmol) in MeOH (50 mL) was added 4-methylbenzenesμLfonic acid (100.02 mg, 580.84 μmol) at 20° C. The mixture was stirred for 12 hrs. TLC (ethyl acetate/MeOH=5:1, Rf=0.4) showed the starting material was consumed and new spot generated. The mixture was concentrated under reduced pressure to give a residue. The residue was purified by column chromatography on silica gel (eluting with ethyl acetate/methanol=20/1 to 5/1) to give D-4 (1.2 g, 26.85% yield) as a colorless oil.

¹H NMR (400 MHz, CHLOROFORM-d) δ 1.35 (1, J=7.00 Hz, 12H), 1.75-1.89 (m, 2H), 2.00-2.13 (m, 2H), 2.38-2.47 (m, 1H), 3.67 (t, J=5.94 Hz, 2H), 4.19 (dq, J=13.80, 7.03 Hz, 8H)

### General Procedure for Preparation of Compound D-5-ET43318-152

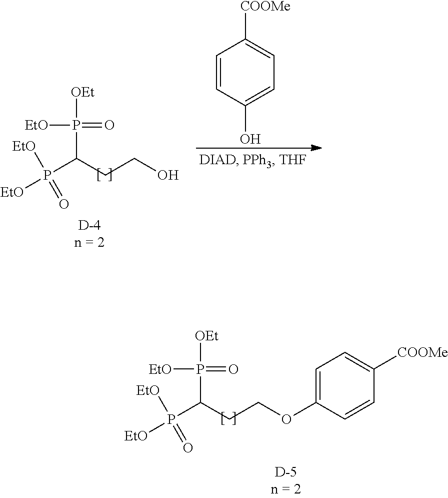

To a solution of PPh₃ (908.91 mg, 3.47 mmol) in THF (10 mL) was added DIAD (700.71 mg, 3.47 mmol) dropwise at 0° C. under nitrogen. After stirring for 30 min, D-4 (800 mg, 2.31 mmol) and methyl 4-hydroxybenzoate (527.23 mg, 3.47 mmol) was added into. The mixture was warmed up to 20° C. and stirred for 12 hrs. LCMS showed the starting material was consumed and 31.9% of product with desired Ms was detected. TLC (ethyl acetate/MeOH=5/1, Rf=0.65) showed new spot generated. The reaction mixture was poured into water (100 mL) and extracted with ethyl acetate (3×30 mL). The organic layers were combined, washed with brine (2×10 mL), dried over Na₂SO₄, filtered and concentrated under reduced pressure to give a residue. The residue was purified by column chromatography on silica gel (eluting with ethyl acetate/methanol=1/0 to 10/1) to give D-5 (450 mg, 40.55% yield) as a colorless oil.

¹H NMR (400 MHz, CHLOROFORM-d) δ 1.34 (td. J=7.04, 1.44 Hz, 12H), 2.07-2.20 (m, 4H), 2.29-2.45 (m, 1H), 3.88 (s, 3H), 4.03 (t, J=5.50 Hz, 2H), 4.15-4.23 (m, 8H), 6.84-6.94 (m, 2H), 7.93-8.03 (m, 2H)

### General Procedure for Preparation of Compound Linker D3-ET43318-158

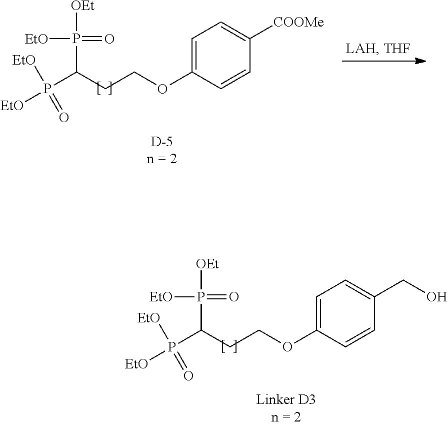 [SMILES: CCOP(=O)(OCC)C(CCOc1ccc(C(=O)OC)cc1)P(=O)(OCC)OCC ; CCOP(=O)(OCC)C(CCOc1ccc(CO)cc1)P(=O)(OCC)OCC]

To a solution of D-5 (600 mg, 1.25 mmol) in THF (20 mL) was added LiAlH₄ (142.20 mg, 3.75 mmol) at 0° C. The mixture was warmed up to 20° C. and stirred for 2 hrs. LCMS showed the starting material was consumed and 31.2% of product with desired Ms was detected. The mixture was cooled to 0° C., H₂O (0.142 mL) was added into dropwise slowly, then NaOH solution (0.142 mL, 15%) was added into dropwise and stirred for 30 min, then H₂O (0.426 mL) was added into. The mixture was warmed up to 25° C. and stirred for 1 hour. The mixture was filtered and the filter cake was washed with THF (2×25 mL). The filtrate was concentrated under reduced pressure to give a residue. The residue was purified by column chromatography on silica gel (eluting with ethyl acetate/methanol=1/0 to 10/1) to give Linker D3 (200 mg, 28.32% yield) as a colorless oil.

¹H NMR (400 MHz, CHLOROFORM-d) δ 1.34 (td, J=7.07, 1.50 Hz, 12H), 2.06-2.21 (m, 4H), 2.30-2.46 (m, 1H), 3.98 (t, J=5.69 Hz, 2H), 4.14-4.24 (m, 8H), 4.62 (s, 2H), 6.84-6.90 (m, 2H), 7.27-7.31 (m, 2H)

### General Procedure for Preparation of Compound D-6-ET43318-163

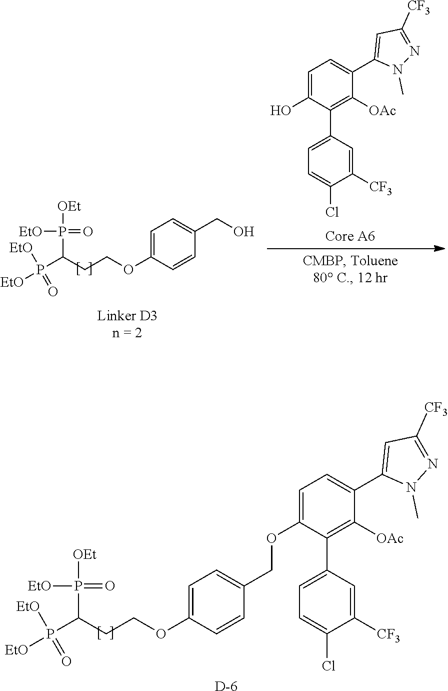

To a solution of Linker D3 (180 mg, 397.87 μmol), Core A6 (209.54 mg, 437.65 μmol) in toluene (3 mL) was added CMBP (144.04 mg, 596.80 μmol) dropwise at 20° C. under nitrogen. The mixture was heated to 80° C. and stirred for 12 hrs. LCMS showed the starting material was remaining and 41.9% of product with desired Ms was detected. The reaction mixture was poured into water (60) mL) and extracted with ethyl acetate (2×20 mL). The organic layers were combined, washed with brine (2×10 mL), dried over Na₂SO₄, filtered and concentrated under reduced pressure to give a residue. The residue was purified by column chromatography on silica gel (eluting with ethyl acetate/methanol=1/0 to 10/1) to give D-6 (300 mg, 33.03% yield) as a yellow oil.

¹H NMR (400 MHz, CHLOROFORM-d) δ 0.94 (s, 12H), 1.77 (s, 3H), 2.06-2.18 (m, 4H), 2.29-2.46 (m, 1H), 3.79-3.86 (m, 3H), 3.97 (br t, J=5.44 Hz, 2H), 4.14-4.24 (m, 8H), 4.99-5.08 (m, 2H), 6.45-6.57 (m, 1H), 6.80-6.88 (m, 2H), 7.08 (d, J=8.63 Hz, 1H), 7.12-7.19 (m, 2H), 7.32 (d, J=8.63 Hz, 1H), 7.42-7.48 (m, 1H), 7.49-7.56 (m, 1H), 7.71 (d, J=1.38 Hz, 1H)

### General Procedure for Preparation of Compound D-7 (NUCC-0226597)-ET43318-169

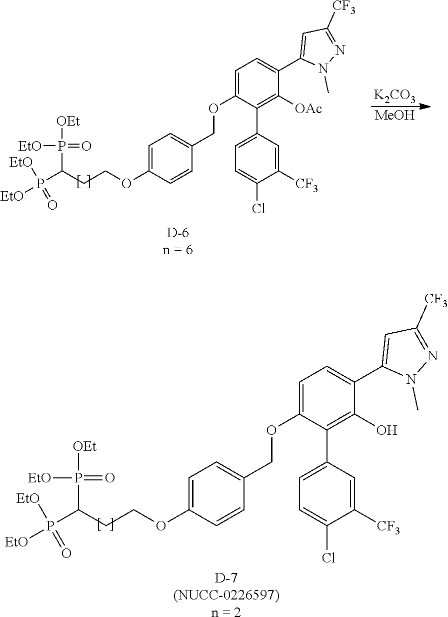

To a solution of D-6 (200 mg, 219.02 μmol) in MeOH (0.5 mL) and H₂O (0.5 mL) was added K₂CO₃ (90.81 mg, 657.05 μmol) at 20° C. The mixture was stirred for 12 hrs. LCMS showed the starting material was consumed and 93.3% of product with desired Ms was detected. The mixture was filtered to give a filtrate. The filtrate was purified by prep-HPLC to give D-7 (87.4 mg, 45.21% yield) as an off-white solid.

Method of prep-HPLC:
Instrument: Gilson 281 semi-preparative HPLC system
Mobile phase: A: 10 mM NH₄HCO₃ in H₂O; B: Acetonitrile
Column: Waters Xbridge BEH C18 100*30 mm*10 um
Flow rate: 25 mL/min
Monitor wavelength: 220&254 nm

| Time | B % |
|---|---|
| 0.0 | 60 |
| 10.0 | 90 |
| 10.1 | 90 |
| 10.3 | 100 |
| 13.2 | 100 |
| 13.3 | 60 |
| 14.5 | 60 |

¹H NMR (400 MHz, CHLOROFORM-d) δ 1.33 (td, J=7.06, 1.41 Hz, 12H), 2.02-2.19 (m, 4H), 2.26-2.44 (m, 1H), 3.84 (s, 3H), 3.96 (t, J=5.62 Hz, 2H), 4.11-4.22 (m, 8H), 5.02 (s, 2H), 5.40 (s, 1H), 6.56 (s, 1H), 6.76 (d, J=8.56 Hz, 1H), 6.83 (d, J=8.68 Hz, 2H), 7.14 (d, J=8.68 Hz, 2H), 7.20 (d, J=8.56 Hz, 1H), 7.49-7.55 (m, 1H), 7.56-7.61 (m, 1H), 7.78 (d, J=1.83 Hz, 1H)

$$LCMS ( ESI + ) : RT = 3.072 min , m / z 871.1 {( M + H )}^{+} .$$

### 5_95AB_6min-220-254-ELSD

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

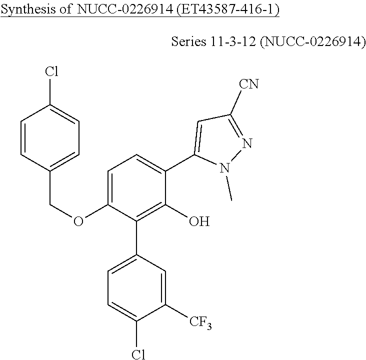 [SMILES: Cn1nc(C#N)cc1-c1ccc(OCc2ccc(Cl)cc2)c(-c2ccc(Cl)c(C(F)(F)F)c2)c1O]

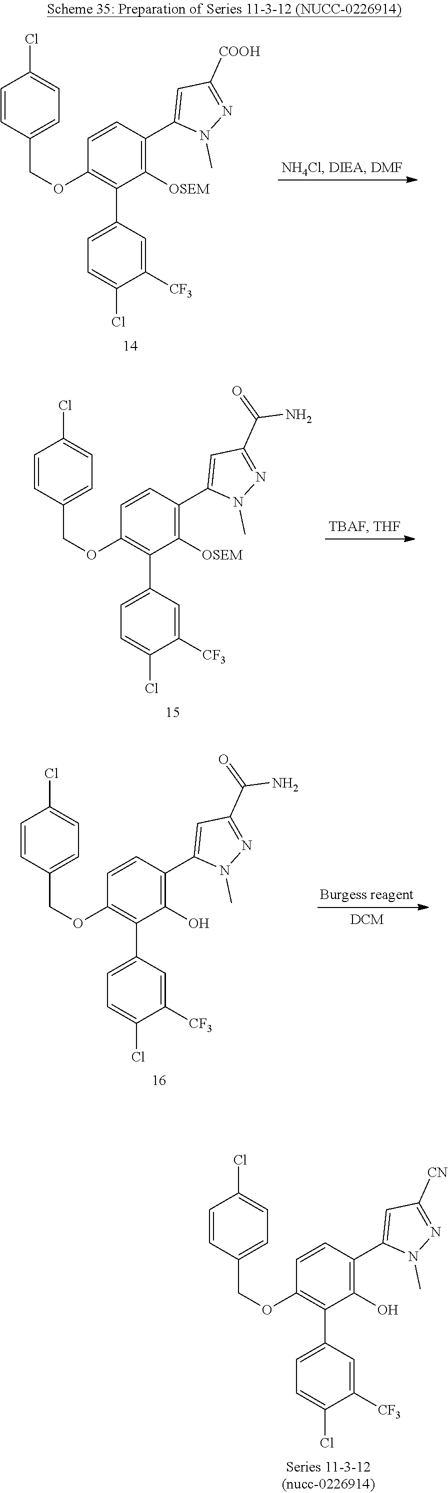

### Chemical Synthesis

### LCMS Methods:

### Method 1: 5_95AB_2min

LC/MS (The column used for chromatography was a Kinetex EVO C18 2.1*30 mm, Sum. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, S-95% B (0.01-0.70 min), 95% B (0.70-1.16 min), 95-5% B (1.16-1.50 min). The flow rate was 1.5 mL/min.

### Method 2: 5_95AB_6min-220-254-ELSD

LC/MS (The gradient was 5% B in 0.40 min and 5-95% B in 2.60 min, hold on 95% B in 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Kinetex C18 2.1*50 mm, Sum. Detection methods are diode array (DAD), and evaporative light scattering detection (ELSD). MS mode was positive electrospray ionization. MS range was 100-1000.

### Experimentals for Largest Scale Run

### General Procedure for Preparation of Compound 15-ET43587-401

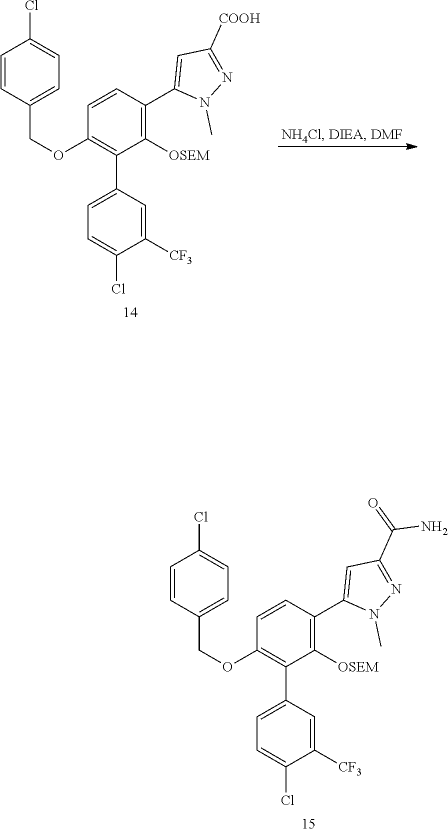

To a solution of Compound 14 (200 mg, 299.59 μmol, 1 eq), NH₄Cl (80.13 mg, 1.50 mmol, 5 eq) and DIEA (116.16 mg, 898.78 μmol, 156.55 μL, 3 eq) in DMF (4 mL) was added HOBt (60.72 mg, 449.39 μmol, 1.5 eq), the mixture was stirred at 25° C. for 30 mins. Then EDCI (86.15 mg, 449.39 μmol, 1.5 eq) was added at 0° C. The mixture was stirred at 25° C. for 12 hrs. LCMS showed all the starting material was consumed and a new peak with desired product MS was detected. The reaction mixture was diluted with water (10 mL) and extracted with ethyl acetate (3×10 mL). The organic layer was separated and the combined organic layer was washed with brine (2×10 ml), dried over Na₂SO₄, filtered and concentrated under reduced pressure to give a residue, which was purified by chromatography on silica gel (eluted with petroleum ether/ethyl acetate=20/1 to 1/3) to give Compound 15 (170 mg, yield 76.61%) as a white solid.

3H NMR (ET43587-401-P1A, 400 MHz, CHLOROFORM-d) δ 0.10 (, 9H), 0.51-0.59 (m, 2H), 2.85-2.96 (m, 2H), 3.85 (s, 3H), 4.41 (s, 2H), 5.06 (s, 2H), 5.34 (br s, 1H), 6.75 (br s, 1H), 6.85 (s, 1H), 6.91 (d, J=8.63 Hz, 1H), 7.17 (d, J=8.50 Hz, 2H), 7.28-7.35 (m, 3H), 7.53-7.61 (m, 2H), 7.84 (d, J=1.63 Hz, 1H)

### General Procedure for Preparation of Compound 16-ET43587-406

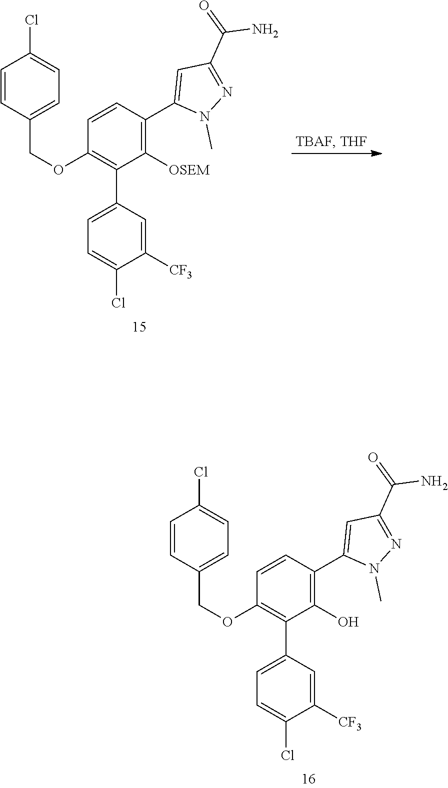

A mixture of Compound 15 (100 mg, 150.02 μmol, 1 eq) in TBAF (1 M, 3.00 ml, 20 eq) was stirred at 25° C. for 20 hrs. LCMS showed all the starting material was consumed and a new peak with desired product MS was detected. The reaction mixture was diluted with water (10 mL) and extracted with ethyl acetate (3×10 mL). The organic layer was separated and the combined organic layer was washed with brine (10 mL), dried over Na₂SO₄, filtered and concentrated under reduced pressure to give a residue, which was purified by chromatography on silica gel (eluted with petroleum ether/ethyl acetate=20/1 to 1/2) to give Compound 16 (50 mg, yield 55.93%) as a yellow solid.

$$LCMS ( ESI + ) : RT = 0.846 min , m / z 536.1 {( M + H )}^{+} .$$

5_95AB_2_min: LC/MS (The column used for chromatography was a Kinetex EVO C18 2.1*30 mm, Sum. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min. 5-95% B (0.01-0.70 min), 95% B (0.70-1.16 min), 95-5% B (1.16-1.50 min). The flow rate was 1.5 mL/min.

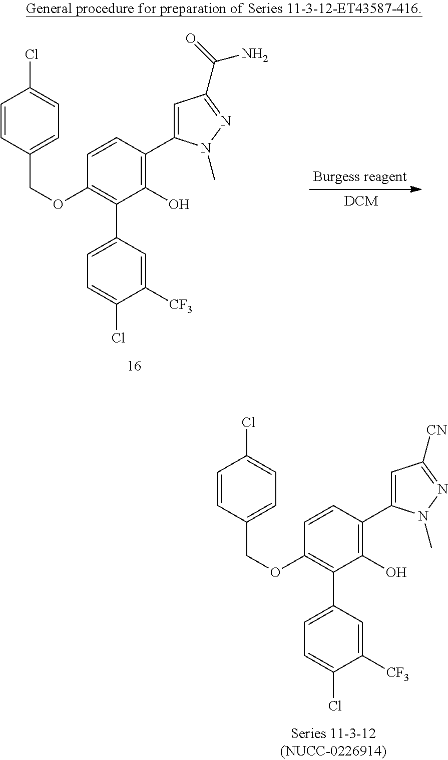 [SMILES: Cn1nc(C(N)=O)cc1-c1ccc(OCc2ccc(Cl)cc2)c(-c2ccc(Cl)c(C(F)(F)F)c2)c1O ; Cn1nc(C#N)cc1-c1ccc(OCc2ccc(Cl)cc2)c(-c2ccc(Cl)c(C(F)(F)F)c2)c1O]

To a solution of Compound 16 (50 mg, 93.23 μmol, 1 eq) in DCM (1 mL) was added burgess reagent (33.32 mg, 139.84 μmol, 1.5 eq) at 0° C. The mixture was stirred at 25° C. for 3 hrs. LCMS showed all the starting material was consumed and a new peak with desired product MS was detected. The mixture was purified by Prep-HPLC (column: Waters Xbridge Prep OBD C18 150*40 mm*10 um; mobile phase: [water (NH₄HCO₃)-ACN]; B %: 55%-85%, 8 min) and lyophilized to give Series Nov. 3, 2012 (11.2 mg, yield 23.18%) as a white solid.

¹H NMR (ET43587-416-P1A, 400 MHz, CHLOROFORM-d) δ 3.85 (s, 3H), 5.06 (s, 2H), 6.70 (s, 1H), 6.73 (d, J=8.63 Hz, 1H), 7.16 (d, J=8.38 Hz, 2H), 7.21 (d, J=8.63 Hz, 1H), 7.29-7.34 (m, 2H), 7.53 (dd, J=8.13, 2.00 Hz, 1H), 7.65 (d, J=8.25 Hz, 1H), 7.79 (d, J=1.88 Hz, 1H)

$$LCMS ( ESI + ) : RT = 3.252 min , m / z 518. {( M + H )}^{+} .$$

5_95AB_6min-220-254-ELSD: LC/MS (The gradient was 5% B in 0.40 min and 5-95% B in 2.60 min, hold on 95% B in 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Kinetex C18 2.1*50 mm, 5 um. Detection methods are diode array (DAD), and evaporative light scattering detection (ELSD). MS mode was positive electrospray ionization. MS range was 100-1000.

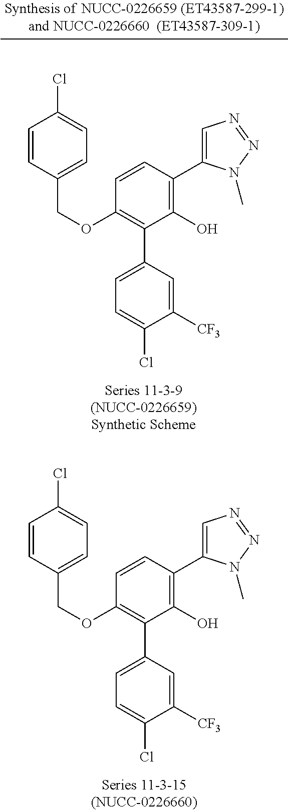 [SMILES: Cn1nncc1-c1ccc(OCc2ccc(Cl)cc2)c(-c2ccc(Cl)c(C(F)(F)F)c2)c1O ; Cn1nncc1-c1ccc(OCc2ccc(Cl)cc2)c(-c2ccc(Cl)c(C(F)(F)F)c2)c1O]

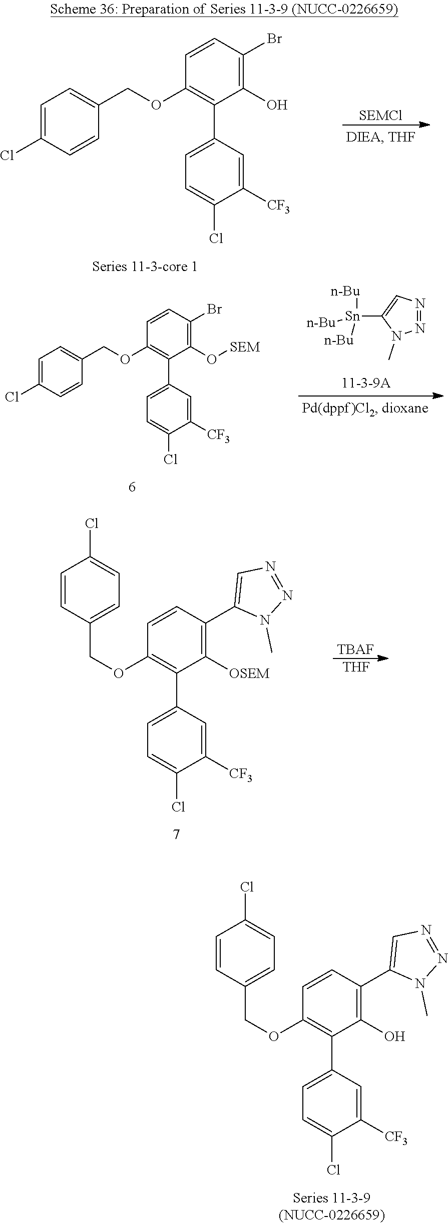

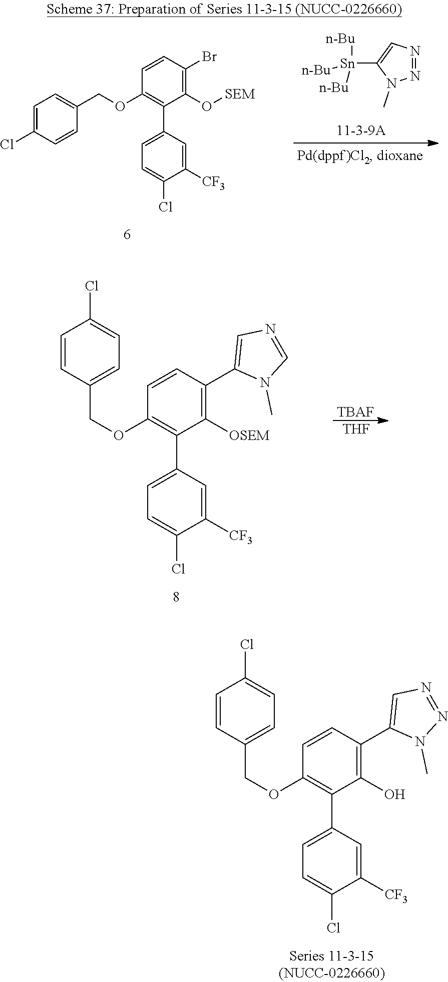

### LCMS Methods:

### Method 1: 5_95CD_6min-220-254-ELSD

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was an Xbridge C18 2.1*50 mm column (5 um particles). Detection methods are diode array (DAD) and evaporative light scattering (ELSD) detection. MS mode was positive electrospray ionization. MS range was 100-1000.

We take Series 11-3-9 (NUCC-0226659) as example.

### Experimentals for Largest Scale Run

### General Procedure for Preparation of Compound 6-ET43587-275

The reactions were conducted in parallel but combined for purification.

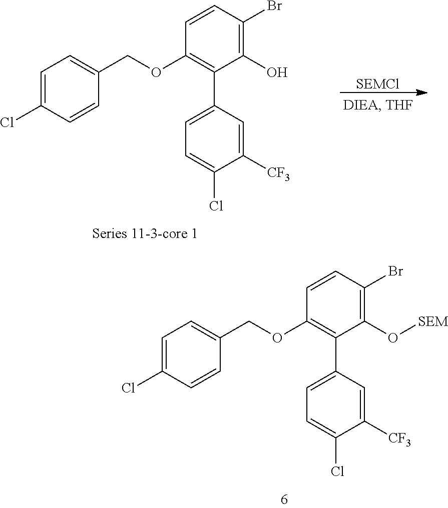 [SMILES: Oc1c(Br)ccc(OCc2ccc(Cl)cc2)c1-c1ccc(Cl)c(C(F)(F)F)c1 ; "SEMCl" ; C[Si](C)(C)CCOCOc1c(Br)ccc(OCc2ccc(Cl)cc2)c1-c1ccc(Cl)c(C(F)(F)F)c1]

To a solution of Series 11-3-core 1 (100 mg, 203.21 μmol, 1 eq) and DIEA (78.79 mg, 609.62 μmol, 106.18 μL, 3 eq) in DCM (1 mL) was added SEMCl (67.76 mg, 406.41 μmol, 71.93 μL, 2 eq) at 0° C. The reaction was stirred at 25° C. for 12 hrs. TLC (petroleum ether/ethyl acetate=10/1) showed the starting material (R_{f}=0.45) was consumed and a new spot (R_{f}=0.5) was generated. Three additional reactions were set up as detailed above and all four reaction mixtures were combined. The combined reaction mixture was concentrated under reduced pressure to give a residue, which was purified by prep-TLC (petroleum ether/ethyl acetate=10/1) to give Compound 6 (340 mg, yield 60.49%) as a white solid.

¹H NMR (ET43587-275-P2A, 400 MHz, CHLOROFORM-d) δ −0.045 (s, 9H), 0.62-0.71 (m, 2H), 3.20-3.28 (m, 2H), 4.83 (s, 2H), 4.98 (s, 2H), 6.73 (d, J=8.88 Hz, 1H), 7.12 (d, J=8.50 Hz, 2H), 7.28-7.31 (m, 2H), 7.51-7.56 (m, 3H), 7.81 (s, 1H)

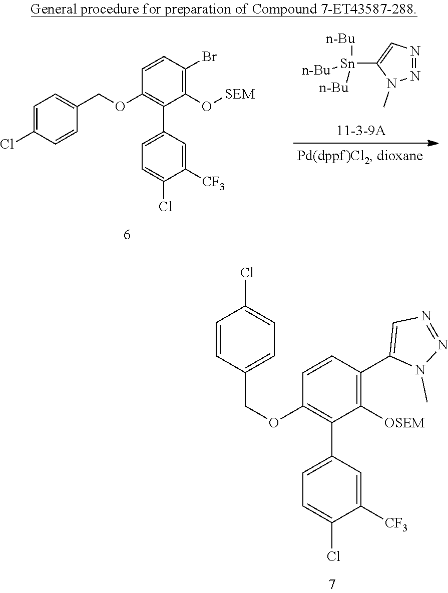

To a solution of Compound 6 (70 mg, 112.47 μmol, 1 eq) and Compound 11-3-9A (167.42 mg, 449.89 μmol, 4 eq) in dioxane (4 mL) was added Pd(dppf)Cl₂ (16.46 mg, 22.49 μmol, 0.2 eq) under nitrogen, the mixture was stirred at 100° C. for 12 hrs. TLC (petroleum ether/ethyl acetate=1/1) showed the starting material (R_{f}=0.9) was consumed and a new spot (R_{f}=0.3) was generated. The reaction mixture was filtered and concentrated under reduced pressure to give a residue, which was purified by prep-TLC (petroleum ether/ethyl acetate=1/1) to give Compound 7 (120 mg, yield 76.87%) as a white solid.

1H NMR (ET43587-288-P1A. 400 MHz, METHANOL-d₄) δ −0.1 (s, 9H), 0.49-0.57 (m, 2H), 2.87-2.96 (m, 2H), 4.04 (s, 3H), 4.38 (s, 2H), 5.14 (s, 2H), 7.16 (d, J=8.63 Hz, 1H), 7.23-7.34 (m, 4H), 7.42 (d, J=8.63 Hz, 1H), 7.64-7.72 (m, 2H), 7.76-7.81 (m, 1H), 7.86 (s, 1H)

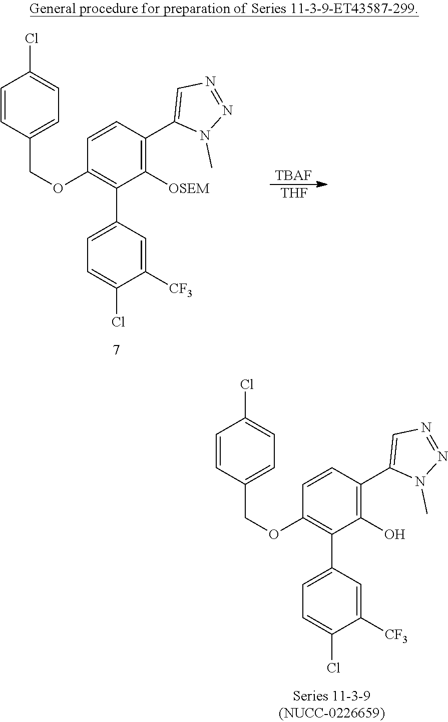

To a solution of Compound 7 (120 mg, 192.14 μmol, 1 eq) in THF (1 mL) was added TBAF (1 M, 1.44 mL, 7.5 eq) at 0° C., the mixture was stirred at 65° C. for 12 hrs. LCMS showed the starting material was consumed and 80% of product with desired MS was detected. The reaction mixture was filtered and concentrated under reduced pressure to give a residue, which was purified by prep-HPLC (column: Waters Xbridge Prep OBD C18 150*40 mm*10 um; mobile phase: [water (NH₄HCO₃)-ACN]; B %: 50%-85%, 8 min) to give Series 11-3-9 (34.1 mg, yield 35.37%) as a white solid.

¹H NMR (ET43587-299-P1A, 400 MHz, METHANOL-d₄) δ 3.97 (s, 3H), 5.10 (s, 2H), 6.87 (d, J=8.63 Hz, 1H), 7.19-7.32 (m, 5H), 7.58-7.80 (m, 4H).

$$LCMS ( ESI + ) : RT = 3.516 min , m / z 494.1 {( M + H )}^{+} .$$

5_95CD_6min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was an Xbridge C18 2.1*50 mm column (5 um particles). Detection methods are diode array (DAD) and evaporative light scattering (ELSD) detection. MS mode was positive electrospray ionization. MS range was 100-1000.

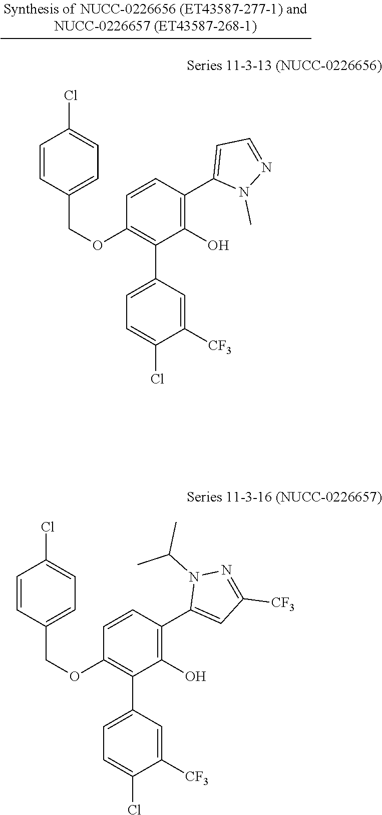 [SMILES: Cn1nccc1-c1ccc(OCc2ccc(Cl)cc2)c(-c2ccc(Cl)c(C(F)(F)F)c2)c1O ; CC(C)n1nc(C(F)(F)F)cc1-c1ccc(OCc2ccc(Cl)cc2)c(-c2ccc(Cl)c(C(F)(F)F)c2)c1O]

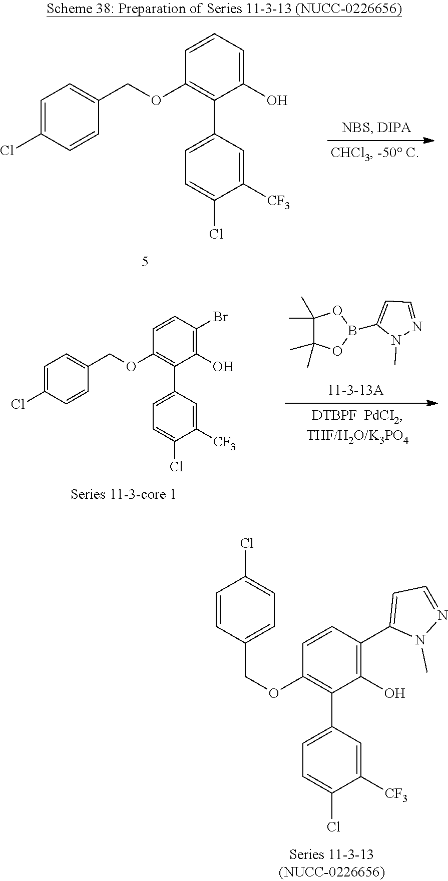 [SMILES: Oc1cccc(OCc2ccc(Cl)cc2)c1-c1ccc(Cl)c(C(F)(F)F)c1 ; Oc1c(Br)ccc(OCc2ccc(Cl)cc2)c1-c1ccc(Cl)c(C(F)(F)F)c1 ; Cn1nccc1B1OC(C)(C)C(C)(C)O1 ; Cn1nccc1-c1ccc(OCc2ccc(Cl)cc2)c(-c2ccc(Cl)c(C(F)(F)F)c2)c1O]

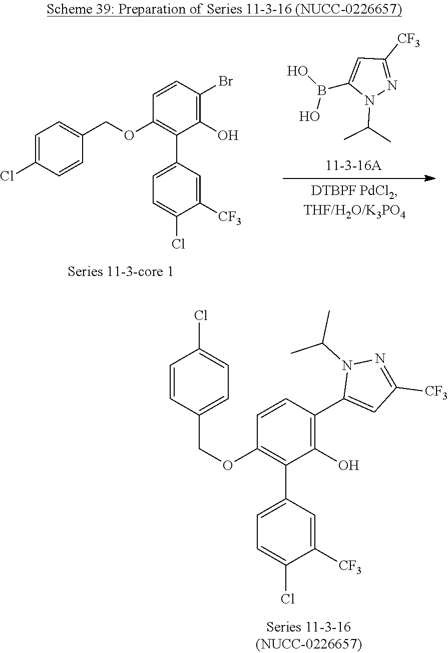 [SMILES: Oc1c(Br)ccc(OCc2ccc(Cl)cc2)c1-c1ccc(Cl)c(C(F)(F)F)c1 ; CC(C)n1nc(C(F)(F)F)cc1B(O)O ; CC(C)n1nc(C(F)(F)F)cc1-c1ccc(OCc2ccc(Cl)cc2)c(-c2ccc(Cl)c(C(F)(F)F)c2)c1O]

### LCMS Methods:

### Method 1: NEG5-95CD_2_min

LC/MS (The column used for chromatography was Xbridge C18 2.1*50 mm, 5 um. Detection methods are diode array (DAD). MS mode was negative electrospray ionization. MS range was 100-1000. Mobile phase A was 10 mm. Ammonium bicarbonate in water, and mobile phase B was HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min) with a hold at 5% B for 0.34 min. The flow rate was 1.5 mL/min.

### Method 2: 5_95CD_6min-220-254-ELSD

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was an Xbridge C18 2.1*50 mm column (5 um particles). Detection methods are diode array (DAD) and evaporative light scattering (ELSD) detection. MS mode was positive electrospray ionization. MS range was 100-1000.

### Method 3: 5_95AB_6min-220-254-ELSD

LC/MS (The gradient was 5% B in 0.40 min and 5-95% B in 2.60 min, hold on 95% B in 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Kinetex C18 2.1*50 mm, 5 um. Detection methods are diode array (DAD), and evaporative light scattering detection (ELSD). MS mode was positive electrospray ionization. MS range was 100-1000.

### Experimentals for Largest Scale Run

### General Procedure for Preparation of Series 11-3-core 1-ET42365-239

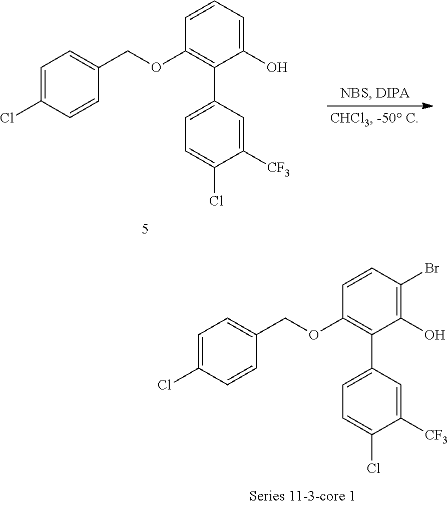 [SMILES: Oc1cccc(OCc2ccc(Cl)cc2)c1-c1ccc(Cl)c(C(F)(F)F)c1 ; Oc1c(Br)ccc(OCc2ccc(Cl)cc2)c1-c1ccc(Cl)c(C(F)(F)F)c1]

To a solution of Compound 5 (4 g, 9.68 mmol, 1.25 eq) and DIPA (1.57 g, 15.49 mmol, 2.19 mL, 2 eq) in CHCl₃ (80 mL) was added NBS (1.38 g, 7.74 mmol, 1 eq) at −40° C., the reaction was stirred at −40° C. for 4 hours. LCMS showed the starting material was consumed and a major new peak was detected. The reaction was diluted with water (100 mL) and was extracted with DCM (3×50 mL). The combined organic layers were washed with brine (100 mL) and dried over Na₂SO₄ and concentrated under reduced pressure to give the crude product which was purified by column chromatography on silica gel (eluted with petroleum ether/ethyl acetate=30/1 to 5/1) to give Series 11-3-core 1 (2.5 g, yield 55.76%) as a yellow solid.

$$LCMS ( ESI + ) : RT = 1.042 min , m / z 490.9 {( M - H )}^{-} .$$

NEG5-95CD_2_min: LC/MS (The column used for chromatography was Xbridge C18 2.1*50 mm, 5 um. Detection methods are diode array (DAD). MS mode was negative electrospray ionization. MS range was 100-1000. Mobile phase A was 10 mm. Ammonium bicarbonate in water, and mobile phase B was HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min) with a hold at 5% B for 0.34 min. The flow rate was 1.5 mL/min.

### General Procedure for Preparation of Series 11-3-2013-ET43587-277

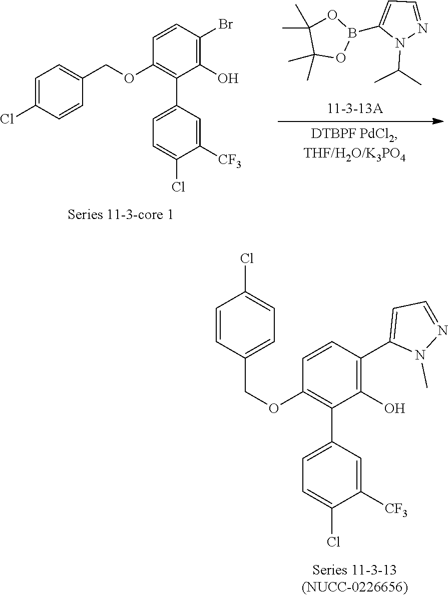 [SMILES: Oc1c(Br)ccc(OCc2ccc(Cl)cc2)c1-c1ccc(Cl)c(C(F)(F)F)c1 ; CC(C)n1nccc1B1OC(C)(C)C(C)(C)O1 ; Cn1nccc1-c1ccc(OCc2ccc(Cl)cc2)c(-c2ccc(Cl)c(C(F)(F)F)c2)c1O]

To a solution of Series 11-3-core1 (150 mg, 304.81 μmol, 1 eq), 11-3-13A (164.89 mg, 792.50 μmol, 2.6 eq) and K₃PO₄ (194.10 mg, 914.42 μmol, 3 eq) in a mixture solution of THF (5 mL) and H₂O (1 mL) was added ditertbutyl(cyclopentyl)phosphane; dichloropalladium;iron (39.73 mg, 60.96 μmol, 0.2 eq) under nitrogen, the reaction was stirred at 80° C. for 12 hrs. LCMS showed the starting material was consumed and 67.7% of product with desired MS was detected. The reaction mixture was filtered and concentrated under reduced pressure to give a residue, which was purified by prep-HPLC (column: Waters Xbridge Prep OBD C18 150*40 mm*10 um; mobile phase: [water (NH₄HCO₃)-ACN]; B %: 70%-95%, 8 min) to give Series Nov. 3, 2013 (11.5 mg, yield 7.28%) as a white solid.

¹H NMR (ET43587-277-P1A, 400 MHz, METHANOL-d₄) 3.73 (s, 3H), 5.08 (s, 2H), 6.29 (d, J=1.88 Hz, 1H), 6.83 (d, J=8.63 Hz, 1H), 7.19-7.23 (m, 3H), 7.27-7.31 (m, 2H), 7.49 (d, J=2.00 Hz, 1H), 7.58-7.65 (m, 2H), 7.77 (s, 1H)

$$LCMS ( ESI + ) : RT = 3.65 min , m / z 493.1 {( M + H )}^{+} .$$

5_95CD_6min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was an Xbridge C18 2.1*50 mm column (5 um particles). Detection methods are diode array (DAD) and evaporative light scattering (ELSD) detection. MS mode was positive electrospray ionization. MS range was 100-1000.

### General Procedure for Preparation of Series Nov. 3, 2016-ET43587-268

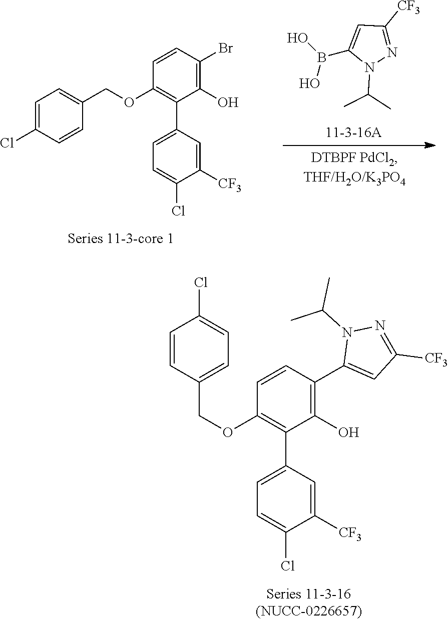 [SMILES: Oc1c(Br)ccc(OCc2ccc(Cl)cc2)c1-c1ccc(Cl)c(C(F)(F)F)c1 ; CC(C)n1nc(C(F)(F)F)cc1B(O)O ; CC(C)n1nc(C(F)(F)F)cc1-c1ccc(OCc2ccc(Cl)cc2)c(-c2ccc(Cl)c(C(F)(F)F)c2)c1O]

To a solution of Series 11-3-core 1 (200 mg, 406.41 μmol, 1 eq), 11-3-16A (117.28 mg, 528.33 μmol, 1.3 eq) and K₃PO₄ (172.54 mg, 812.82 μmol, 2 eq) in a mixture solution of THF (6 ml) and H₂O (1 mL) was added ditert-butyl(cyclopentyl)phosphane;dichloropalladium; iron (26.49 mg, 40.64 μmol, 0.1 eq) under nitrogen, the reaction was stirred at 80° C. for 12 hrs. LCMS showed the starting material was consumed and 41.9% of product with desired MS was detected. The reaction mixture was filtered and concentrated under reduced pressure to give a residue, which was purified by prep-HPLC (column: Waters Xbridge Prep OBD C18 150*40 mm*10 um; mobile phase: [water (NH₄HCO₃)-ACN]; B %: 70%-95%, 8 min) to give Series Nov. 3, 2016 (17.9 mg, yield 7.21%) as a brown solid.

¹H NMR (ET43587-268-P1, 400 MHz, METHANOL-d₄) δ 1.42 (d, J=6.63 Hz, 6H), 4.44 (quin, J=6.63 Hz, 1H), 5.09 (s, 2H), 6.51 (s, 1H), 6.84 (d, J=8.50 Hz, 1H), 7.19-7.24 (m, 3H), 7.27-7.32 (m, 2H), 7.57-7.66 (m, 2H), 7.77 (d, J=1.75 Hz, 1H)

$$LCMS ( ESI + ) : RT = 3.475 min , m / z 589. {( M + H )}^{+} .$$

5_95AB_6min-220-254-ELSD: LC/MS (The gradient was 5% B in 0.40 min and 5-95% B in 2.60 min, hold on 95% B in 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Kinetex C18 2.1*50 mm, Sum. Detection methods are diode array (DAD), and evaporative light scattering detection (ELSD). MS mode was positive electrospray ionization. MS range was 100-1000.

### Synthesis of NUCC-0226652 (ET42365-279-1)

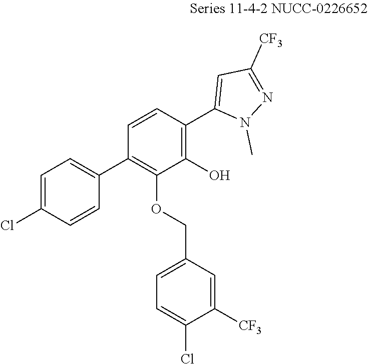 [SMILES: Cn1nc(C(F)(F)F)cc1-c1ccc(-c2ccc(Cl)cc2)c(OCc2ccc(Cl)c(C(F)(F)F)c2)c1O]

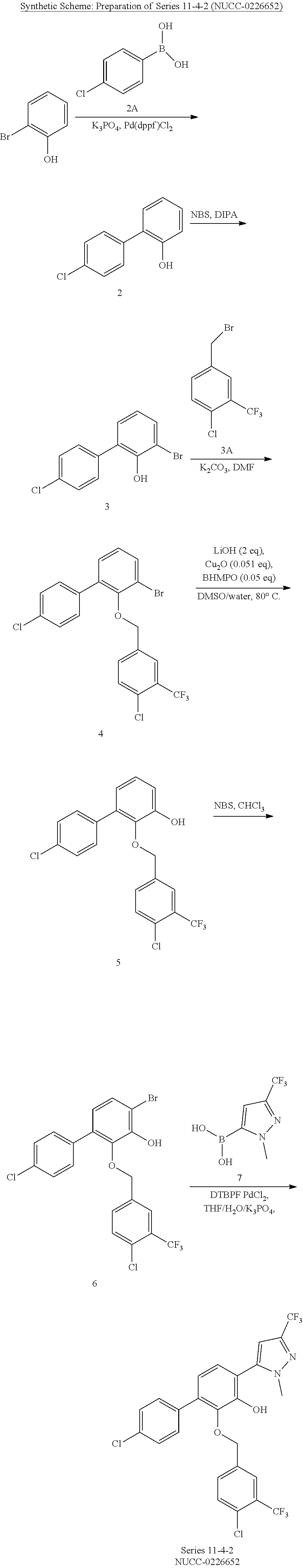

NEG5-95CD_2_min: LC/MS (The column used for chromatography was Xbridge C18 2.1*50 mm, 5 um. Detection methods are diode array (DAD). MS mode was negative electrospray ionization. MS range was 100-1000. Mobile phase A was 10 mM Ammonium bicarbonate in water, and mobile phase B was HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min) with a hold at 5% B for 0.34 min. The flow rate was 1.5 mL/min.

50_100AB_6min-220-254-ELSD: LC/MS (The gradient was 50% B in 0.40 min and 50-100% B at 0.40-3.00 min, hold on 100% B for 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1000.

### Experimentals for Largest Scale Run

### General Procedure for Preparation of Compound 2-ET43365-250

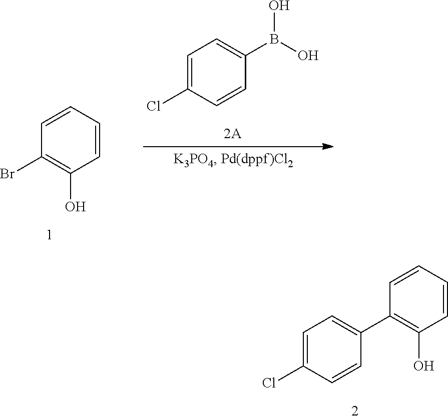 [SMILES: OB(O)c1ccc(Cl)cc1 ; Oc1ccccc1Br ; Oc1ccccc1-c1ccc(Cl)cc1]

To a solution of Compound 1 (0.5 g, 2.89 mmol, 335.57 μL, 1 eq). Compound 2A (587.51 mg, 3.76 mmol, 1.3 eq) and K₃PO₄ (1.53 g, 7.23 mmol, 2.5 eq) in a mixture solution of THF (10 mL) and H₂O (2 mL) was added Pd(dppf)Cl₂·CH₂Cl (177.01 mg, 216.75 μmol, 0.075 eq) under nitrogen, the reaction was stirred at 80° C. for 2 hrs. LCMS showed the starting material was consumed and a new peak with desired product Ms was detected. The crude product diluted with water (50 mL) and extracted with ethyl acetate (3× 50 mL). The organic layer was washed with brine (50 mL), dried over Na₂SO₄, filtered and concentrated under reduced pressure to afford the crude product. The crude product was purified by column chromatography on silica gel (eluted with petroleum ether/ethyl acetate=50/1 to 10/1) to give Compound 2 (500 mg, yield 80.31%) as a yellow solid.

¹H NMR (ET42365-250-P1A, 400 MHz, CHLOROFORM-d) δ 4.96 (s, 1H), 6.85-6.96 (m, 1H), 7.12-7.22 (m, 1H), 7.31-7.42 (m, 2H)

### General Procedure for Preparation of Compound 3-ET42365-256

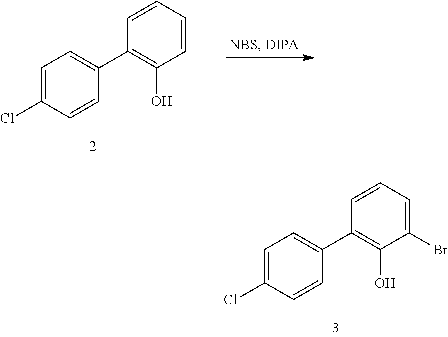 [SMILES: Oc1ccccc1-c1ccc(Cl)cc1 ; Oc1c(Br)cccc1-c1ccc(Cl)cc1]

To a solution of Compound 2 (500 mg, 2.44 mmol, 1 eq) and DIPA (494.45 mg, 4.89 mmol, 690.57 μL, 2 eq) in CHCl₃ (12.5 mL) was added 1-bromopyrrolidine-2,5-dione (413.10 mg, 2.32 mmol, 0.95 eq) at −40° C., the reaction was stirred at −40° C. for 4 hrs. LCMS showed about 9.6% of the starting material was remaining and a new peak (61.8%) was detected. The reaction was diluted with water (50 mL) and was extracted with DCM (3×30 mL). The combined organic layer were washed with brine (50 mL) and dried over Na₂SO₄ and concentrated to give crude product. The crude product was purified by column chromatography on silica gel (eluted with petroleum ether/ethyl acetate=50/1 to 10/1) to give Compound 3 (500 mg, yield 64.96%) as a yellow solid.

¹H NMR (ET42365-256-P1A, 400 MHz, CHLOROFORM-d) δ 5.68 (s, 1H), 6.90 (t, J=7.84 Hz, 1H), 7.24 (dd, J=7.65, 1.38 Hz, 1H), 7.39-7.45 (m, 2H), 7.46-7.52 (m, 3H)

### General Procedure for Preparation of Compound 4-ET42365-257

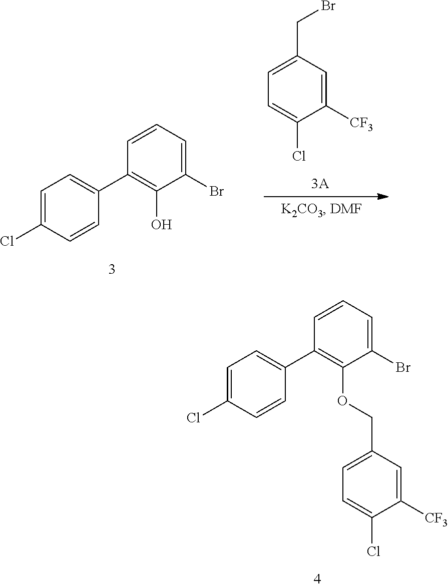 [SMILES: Oc1c(Br)cccc1-c1ccc(Cl)cc1 ; FC(F)(F)c1cc(CBr)ccc1Cl ; FC(F)(F)c1cc(COc2c(Br)cccc2-c2ccc(Cl)cc2)ccc1Cl]

To a solution of Compound 3 (500 mg, 1.76 mmol, 1 eq) in DMF (12 mL) were added K₂CO₃ (487.42 mg, 3.53 mmol, 2 eq) and Compound 3A (530.47 mg, 1.94 mmol, 1.1 eq), the reaction was stirred at 25° C. for 12 hrs. LCMS showed the starting material was consumed and a new peak was detected. The crude product diluted with water (50 mL) and extracted with ethyl acetate (3×30 mL). The organic layer was washed with brine (50 mL), dried over anhydrous sodium sulfate, filtered and concentrated under reduced pressure to afford Compound 4 (700 mg, yield 75.04%) as a yellow oil. The product was used to the next step directly without further purification.

¹H NMR (ET42365-257-P1A, 400 MHz, CHLOROFORM-d) δ 4.59 (s, 2H), 7.08-7.15 (m, 1H), 7.25 (dd, J=8.22, 1.82 Hz, 1H), 7.27-7.30 (m, 1H), 7.35-7.48 (m, 6H), 7.60 (dd, J=7.97, 1.57 Hz, 1H)

### General Procedure for Preparation of Compound 5-ET42365-260

The reactions were conducted in parallel but combined for purification.

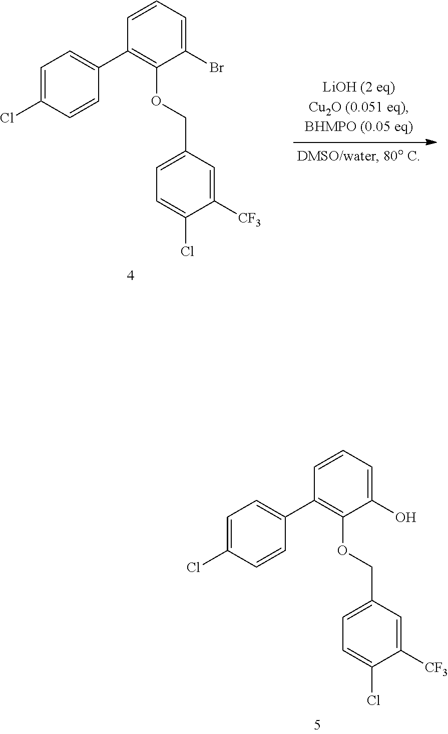 [SMILES: FC(F)(F)c1cc(COc2c(Br)cccc2-c2ccc(Cl)cc2)ccc1Cl ; Oc1cccc(-c2ccc(Cl)cc2)c1OCc1ccc(Cl)c(C(F)(F)F)c1]

To a solution of Compound 4 (350 mg, 735.12 μmol, 1 eq), LiOH·H₂O (61.70 mg, 1.47 mmol, 2 eq) in a mixture solution of DMSO (6 mL) and H₂O (1.5 mL) was added Cu₂O (5.26 mg, 36.76 μmol, 3.76 μL, 0.05 eq) and BHMPO (12.06 mg, 36.76 μmol, 0.05 eq) under nitrogen, the reaction was stirred at 80° C. for 12 hrs. LCMS showed about 25% of the starting material was remaining and a new peak (44%) with desired product Ms was detected. Additional one reaction was set up as described above and combined for purification. The combined reaction was diluted with water (30 mL) and extracted with ethyl acetate (3×20 mL). The organic layer was washed with brine (30 mL), dried over anhydrous sodium sulfate, filtered and concentrated under reduced pressure to give the crude product. The crude product was purified by column chromatography on silica gel (eluted with petroleum ether/ethyl acetate=50/1 to 10/1) to give Compound 5 (500 mg, yield 74.07%) as a yellow solid.

¹H NMR (ET42365-260-P1A, 400 MHz, CHLOROFORM-d) δ 4.57 (s, 2H), 5.66 (s, 1H), 6.91 (d, J=7.63 Hz, 1H), 7.02 (d, J=8.00 Hz, 1H), 7.12-7.19 (m, 1H), 7.26 (br d, J=8.13 Hz, 1H), 7.41-7.50 (m, 4H), 7.54 (d, J=8.38 Hz, 2H)

### General Procedure for Preparation of Compound 6-ET42365-274

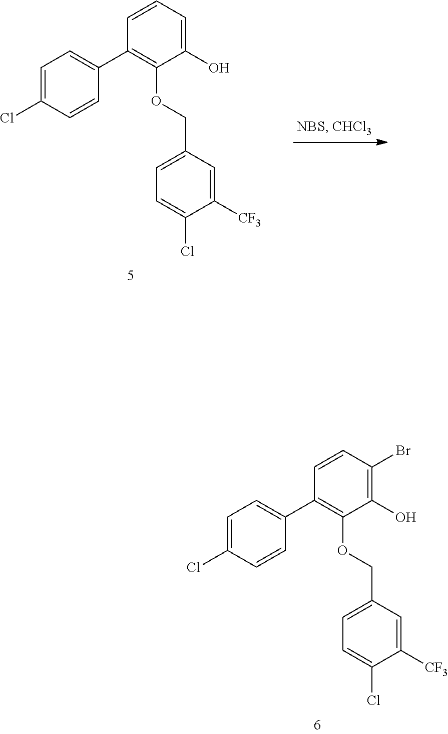 [SMILES: Oc1cccc(-c2ccc(Cl)cc2)c1OCc1ccc(Cl)c(C(F)(F)F)c1 ; Oc1c(Br)ccc(-c2ccc(Cl)cc2)c1OCc1ccc(Cl)c(C(F)(F)F)c1]

To a solution of Compound 5 (400 mg, 968.02 μmol, 1 eq) and DIPA (195.91 mg, 1.94 mmol, 273.61 μL, 2 eq) in CHCl₃ (10 mL) was added 1-bromopyrrolidine-2,5-dione (172.29 mg, 968.02 μmol, 1 eq) in portions at −40° C., the reaction was stirred at −40° C. for 4 hrs. LCMS showed about 5.6% of the starting material was remaining and a new peak with desired product Ms was detected. The reaction was diluted with water (50 mL) and was extracted with DCM (3×30 mL). The combined organic layer were washed with brine (50 mL), dried over Na₂SO₄ and concentrated to give crude product. The crude product was purified by column chromatography on silica gel (eluted with petroleum ether/ethyl acetate=50/1 to 10/1) to give Compound 6 (300 mg, yield 56.68%) as a white solid.

$$LCMS ( ESI + ) : RT = 1.061 min , m / z 490.8 {( M - H )}^{+} .$$

NEG5-95CD_2_min: LC/MS (The column used for chromatography was Xbridge C18 2.1*50 mm, 5 um. Detection methods are diode array (DAD). MS mode was negative electrospray ionization. MS range was 100-1000. Mobile phase A was 10 mM Ammonium bicarbonate in water, and mobile phase B was HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min) with a hold at 5% B for 0.34 min. The flow rate was 1.5 mL/min.

### General Procedure for Preparation of Series 11-4-2-ET42365-279

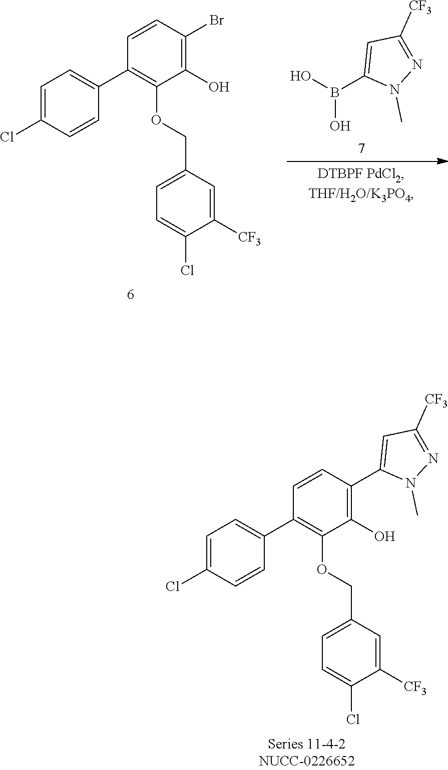 [SMILES: Oc1c(Br)ccc(-c2ccc(Cl)cc2)c1OCc1ccc(Cl)c(C(F)(F)F)c1 ; Cn1nc(C(F)(F)F)cc1B(O)O ; Cn1nc(C(F)(F)F)cc1-c1ccc(-c2ccc(Cl)cc2)c(OCc2ccc(Cl)c(C(F)(F)F)c2)c1O]

To a solution of Compound 6 (200 mg, 406.41 μmol, 1 eq), Compound 7 (118.22 mg, 609.62 μmol, 1.5 eq) and K₃PO₄ (215.67 mg, 1.02 mmol, 2.5 eq) in a mixture solution of THF (6 mL) and H₂O) (1.5 mL) was added ditertbutyl(cyclopentyl)phosphane;dichloro-palladium;iron (52.98 mg, 81.28 μmol, 0.2 eq) under nitrogen, the reaction was stirred at 85° C. for 40 hrs. LCMS showed about 12% of the starting material was remaining and a new peak with desired product Ms was detected. The reaction was filtered and the filtrate was concentrated to give the crude product. The crude product was purified by Prep-HPLC (column: Waters Xbridge Prep OBD C18 150*40 mm*10 um; mobile phase: [water (NH4HCO3)-ACN]; B %: 70%-95%, 8 min) to give Series 11-4-2 (60.1 mg, yield 26.19%) as off-white solid.

¹H NMR (ET42365-279-P1A, 400 MHz, CHLOROFORM-d) δ 3.82 (s, 3H), 4.59 (s, 2H), 6.02 (s, 1H), 6.58 (s, 1H), 6.99 (d, J=8.03 Hz, 1H), 7.11 (d, J=8.16 Hz, 1H), 7.25 (s, 1H), 7.41-7.51 (m, 4H), 7.54-7.61 (m, 2H)

$$LCMS ( ESI + ) : RT = 2.774 min , m / z 561. {( M + H )}^{+} .$$

50_100AB_6min-220-254-ELSD: LC/MS (The gradient was 50% B in 0.40 min and 50-100% B at 0.40-3.00 min, hold on 100% B for 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1000.

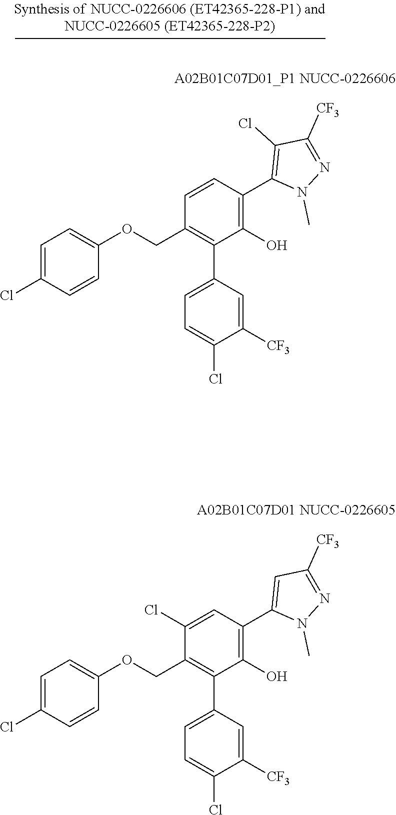 [SMILES: Cn1nc(C(F)(F)F)c(Cl)c1-c1ccc(COc2ccc(Cl)cc2)c(-c2ccc(Cl)c(C(F)(F)F)c2)c1O ; Cn1nc(C(F)(F)F)cc1-c1cc(Cl)c(COc2ccc(Cl)cc2)c(-c2ccc(Cl)c(C(F)(F)F)c2)c1O]

### Synthetic Scheme

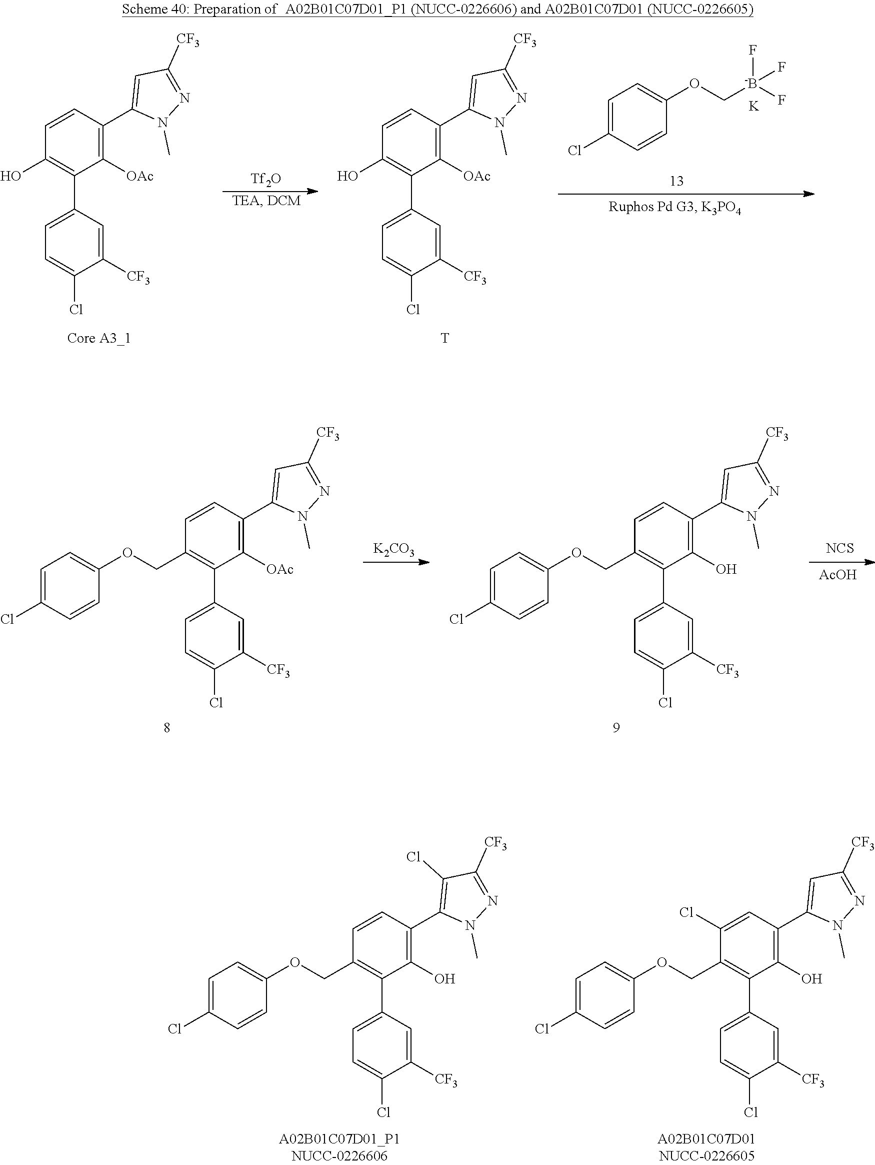

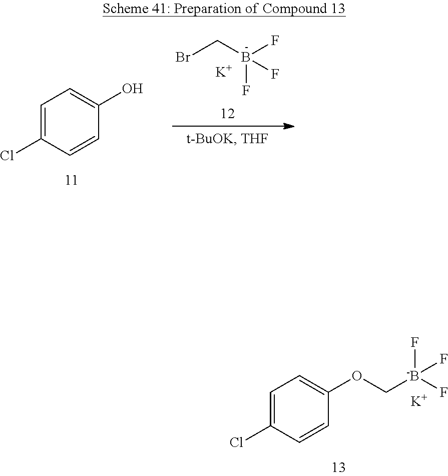

### Chemical Synthesis

### LCMS Methods:

### Method 1:

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 ml/min.

### Method 2:

5-95CD 4.5 min: LC/MS (The column used for chromatography was Xbridge C18 2.1*50 mm, 5 um). Detection methods are diode array (DAD). MS mode was positive electrospray ionization MS range was 100-1000. Mobile phase A was 10 mM ammonium bicarbonate in water, and mobile phase B was HPLC grade acetonitrile. The gradient was 5-95% B in 4.30 min 0.5% B in 0.01 min. 5-95% B (0.01-3.00 min), and hold at 95% B within 0.5 min, 95-5% B (3.50-3.51 min), with a hold at 5% B for 0.79 min. The flow rate was 1.0 mL/min (0.01-4.30 min).

### Method 3:

50_100CD_6min-220-254-ELSD: LC/MS (The gradient was 50% B in 0.40 min and 50-100% B at 0.40-3.40 min, hold on 100% B for 0.45 min, and then 100-50% B in 0.01 min. the flow rate was 0.8 ml/min. Mobile phase A was H₂O+10 mM NH₄HCO₃, mobile phase B was Acetonitrile. The column used for chromatography was a Xbridge C18 2.1*50 mm column (5 um particles). Detection methods are diode array (DAD) and evaporative light scattering (ELSD) detection. MS mode was positive electrospray ionization. MS range was 100-1000.

### Method 4:

5_95CD_6min-220-254-ELSD: LC/MS (The gradient was 5% B in 0.40 min and 5-95% B at 0.40-3.40 min. hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge C18 2.1*50 mm column (Sum particles). Detection methods are diode array (DAD) detection. MS mode was positive electrospray ionization. MS range was 100-1000.

### Experimentals for Largest Scale Run

### General Procedure for Preparation of Compound 13-ET42365-161

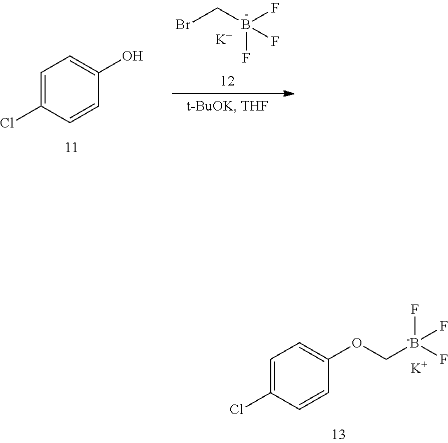

To a solution of Compound 11 (300 mg, 2.33 mmol, 1.2 eq) in THF (6 mL) was added t-BuOK (1 M, 2.33 mL, 1.2 eq) at 0° C., the reaction was stirred at 0° C. for 1 hour, then Compound 12 (390.56 mg, 1.94 mmol, 1 eq) was added and the reaction was stirred at 50° C. for 12 hrs. LCMS showed all the starting material was consumed and two new peak was detected. The reaction was filtered and the solid was collected and dried to give Compound 13 (300 mg, yield 62.08%) as off-white solid.

¹H NMR (ET42365-161-PIB, 400 MHz, DMSO-d6) δ 2.94 (q, J=5.09 Hz, 2H), 6.82-6.89 (m, 2H), 7.17-7.24 (m, 2H)

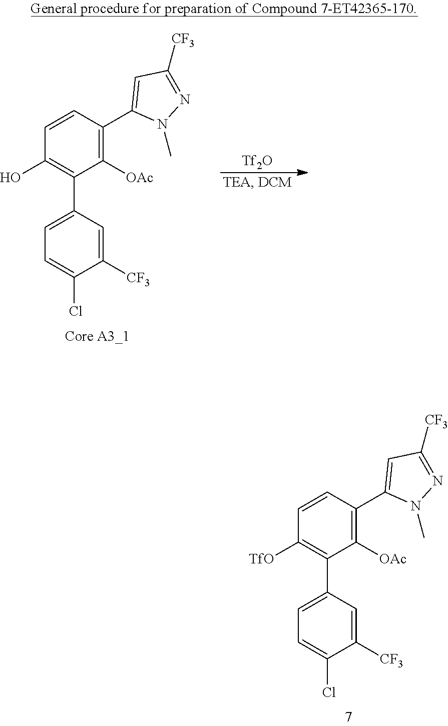 [SMILES: "Tf2O" ; CC(=O)Oc1c(-c2cc(C(F)(F)F)nn2C)ccc(O)c1-c1ccc(Cl)c(C(F)(F)F)c1 ; "TEA, DCM" ; CC(=O)Oc1c(-c2cc(C(F)(F)F)nn2C)ccc(OS(=O)(=O)C(F)(F)F)c1-c1ccc(Cl)c(C(F)(F)F)c1]

To a solution of Core A3_1 (400 mg, 835.47 μmol, 1 eg) and pyridine (264.34 mg, 3.34 mmol, 269.74 μL, 4 eq) in DCM (12 mL) was added Tf₂O (353.58 mg, 1.25 mmol, 206.77 μL, 1.5 eq) dropwise at 0° C., the reaction was stirred at 20° C. for 3 hrs. LCMS showed the starting material was consumed and a new peak with desired product Ms was detected. The reaction was diluted with water (50 mL) and extracted with DCM (3×25 mL). The organic layer was separated and the combined organic layer was washed with brine (30 mL), dried over Na₂SO₄, filtered and concentrated under reduced pressure to give crude product. The crude product was purified by column chromatography on silica gel (eluted with petroleum ether/ethyl acetate=50/1 to 10/1) to give Compound 7 (0.4 g, yield 78.38%) as a white solid.

$$LCMS ( ESI + ) : RT = 0.915 min , m / z 611.1 {( M + H )}^{+} .$$

S-95AB 2 min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Compound 8-ET42365-200

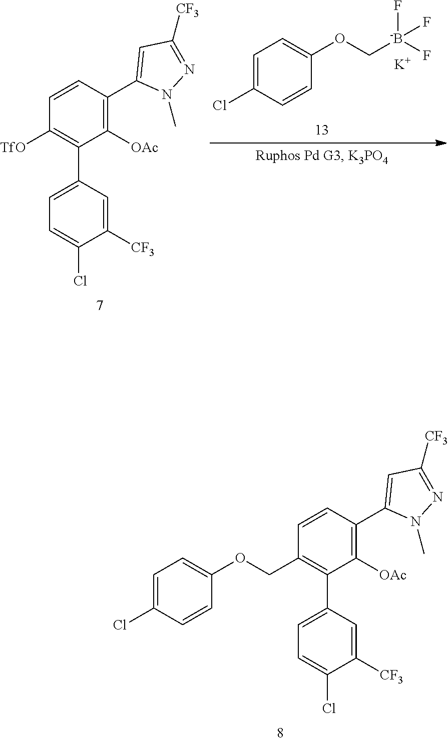

To a solution of Compound 7 (200 mg, 327.42 μmol, 1 eq), Compound 13 (188.32 mg, 757.88 μmol, 2.31 eq) and K₃PO₄ (208.50 mg, 982.26 μmol, 3 eq) in a mixture solution of H₂O (2 mL) and toluene (10 mL) was added RuPhos Pd G3 (54.77 mg, 65.48 μmol, 0.2 eq) under nitrogen, the reaction was stirred at 110° C. for 20 hrs. LCMS showed the starting material was consumed and a new peak with desired product Ms was detected. The reaction was filtered and the filtrate was concentrated to give the crude product. The crude product was purified by Prep-TLC (petroleum ether/ethyl acetate=2/1) to give Compound 8 (50 mg, yield 20.25%) as a yellow solid.

$$LCMS ( ESI + ) : RT = 0.988 min , m / z 603.2 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 ml/min.

### General Procedure for Preparation of Compound 8-ET42365-201

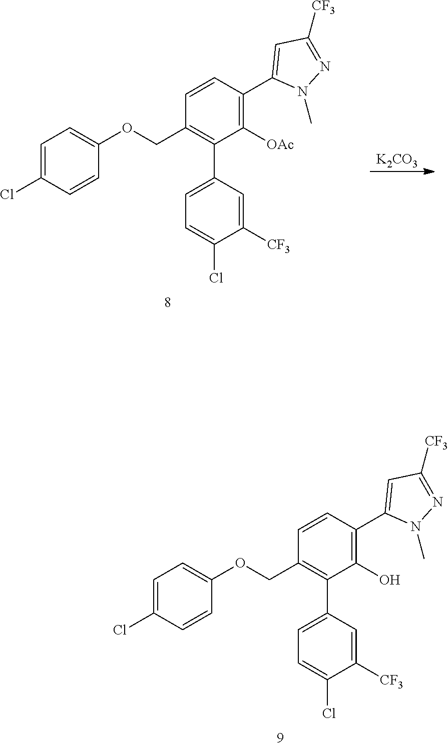

To a solution of Compound 8 (80 mg, 106.08 μmol, 80% purity, 1 eq) in MeOH (3 mL) was added K₂CO₃ (29.32 mg, 212.15 μmol, 2 eq), the reaction was stirred at 20° C. for 12 hrs. LCMS showed the starting material was consumed and a new peak with desired product Ms was detected. The reaction was diluted with water (10 mL) and was extracted with ethyl acetate (3×10 mL). The combined organic layer were washed with brine (10 mL) and dried over Na₂SO₄ and concentrated to give crude product. The crude product was purified by Prep-HPLC (column: Waters Xbridge Prep OBD C18 150*40 mm*10 um; mobile phase: [water (10 mM NH4HCO3)-ACN]; B %: 70%-95%, 8 min) to give Compound 9 (40 mg, yield 67.18%) as a white solid.

$$LCMS ( ESI + ) : RT = 3.108 min , m / z 561. {( M + H )}^{+} .$$

5-95CD_4.5 min: LC/MS (The column used for chromatography was Xbridge C18 2.1*50 mm, 5 um). Detection methods are diode array (DAD). MS mode was positive electrospray ionization. MS range was 100-1000. Mobile phase A was 10 mM ammonium bicarbonate in water, and mobile phase B was HPLC grade acetonitrile. The gradient was 5-95% B in 4.30 min 0.5% B in 0.01 min, 5-95% B (0.01-3.00 min), and hold at 95% B within 0.5 min, 95-5% B (3.50-3.51 min), with a hold at 5% B for 0.79 min. The flow rate was 1.0 mL/min (0.01-4.30 min).

### General Procedure for Preparation of A02B01C07D01_P1 and A02B01C07D01-ET42365-228

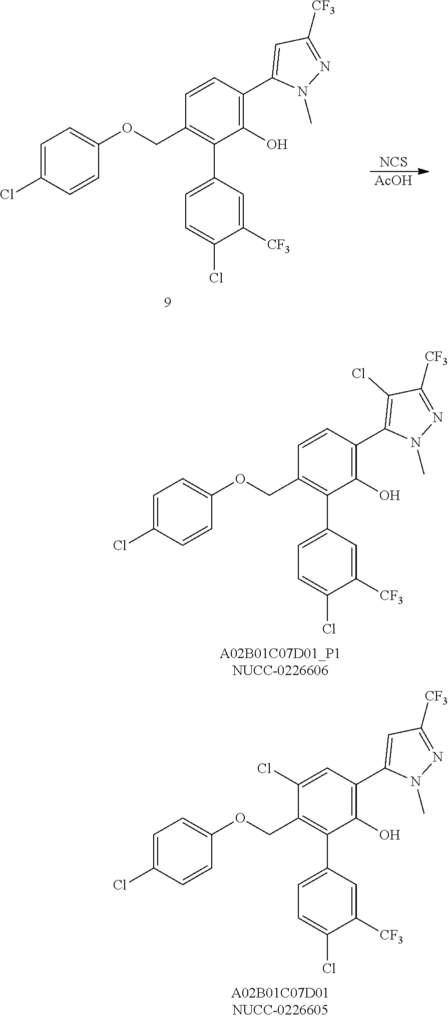 [SMILES: "AcOH" ; Cn1nc(C(F)(F)F)cc1-c1ccc(COc2ccc(Cl)cc2)c(-c2ccc(Cl)c(C(F)(F)F)c2)c1O ; "NCS" ; Cn1nc(C(F)(F)F)c(Cl)c1-c1ccc(COc2ccc(Cl)cc2)c(-c2ccc(Cl)c(C(F)(F)F)c2)c1O ; Cn1nc(C(F)(F)F)cc1-c1cc(Cl)c(COc2ccc(Cl)cc2)c(-c2ccc(Cl)c(C(F)(F)F)c2)c1O]

To a solution of Compound 9 (50 mg, 89.08 μmol, 1 eq) in AcOH (2 mL) was added NCS (11.89 mg, 89.08 μmol, 1 eq) at 0° C., the reaction was stirred at 60° C. for 12 hrs. LCMS showed about 33% of the starting material was remaining and two new peaks with desired Ms were detected. The reaction was concentrated to give crude product. The crude product was purified by Prep-HPLC (column: Waters Xbridge Prep OBD C18 150*40 mm*10 um; mobile phase: [water (10 mM NH₄HCO₃)-ACN]; B %: 70%-90%, 8 min) to give the crude product, which was further purified by Prep-HPLC (column: Phenomenex Luna 80*30 mm*3 um; mobile phase: [water (0.1% TFA)-ACN]; B %: 70%-90%, 8 min) to give A02B01C07D01_P1 (NUCC-0226606) (15.2 mg, yield 27.9%) and A02B01C07D01 (NUCC-0226605) (22.3 mg, yield 41.05%) as white solid.

### A02B01C07D01 (NUCC-0226606):

¹H NMR (ET42365-228-PIB, 400 MHz, CHLOROFORM-d) δ 3.84 (s, 3H), 4.65-4.78 (m, 2H), 4.93 (br s, 1H), 6.75 (d, J=8.88 Hz, 2H), 7.23 (d, J=8.88 Hz, 2H), 7.33-7.43 (m, 2H), 7.54 (br d, J=8.25 Hz, 1H), 7.67 (d, J=8.13 Hz, 1H), 7.76 (d, J=1.38 Hz, 1H)

$$LCMS ( ESI + ) : RT = 2.467 min , m / z 596.9 {( M + H )}^{+} .$$

5_95CD 6 min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge C18 2.1*50 mm column (5 um particles). Detection methods are diode array (DAD) detection. MS mode was positive electrospray ionization. MS range was 100-1000.

### A02B01C07D01 (NUCC-0226605):

¹H NMR (ET42365-228-PIC, 400 MHz, CHLOROFORM-d) δ 3.90 (s, 3H), 4.71-4.78 (m, 1H), 4.79-4.86 (m, 1H), 5.02 (br s, 1H), 6.65 (s, 1H), 6.77 (d, J=9.01 Hz, 2H), 7.18-7.26 (m, 2H), 7.45 (s, 1H), 7.52 (dd, J=8.19, 1.81 Hz, 1H), 7.62 (d, J=8.13 Hz, 1H), 7.76 (d, J=1.63 Hz, 1H)

$$LCMS ( ESI + ) : RT = 2.513 min , m / z 596.9 {( M + H )}^{+} .$$

5_95CD_6min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge C18 2.1*50 mm column (Sum particles). Detection methods are diode array (DAD) detection. MS mode was positive electrospray ionization. MS range was 100-1000.

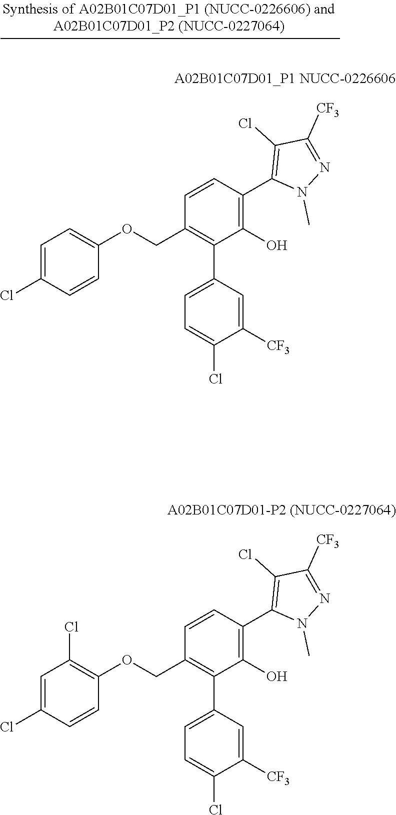 [SMILES: Cn1nc(C(F)(F)F)c(Cl)c1-c1ccc(COc2ccc(Cl)cc2)c(-c2ccc(Cl)c(C(F)(F)F)c2)c1O ; Cn1nc(C(F)(F)F)c(Cl)c1-c1ccc(COc2ccc(Cl)cc2Cl)c(-c2ccc(Cl)c(C(F)(F)F)c2)c1O]

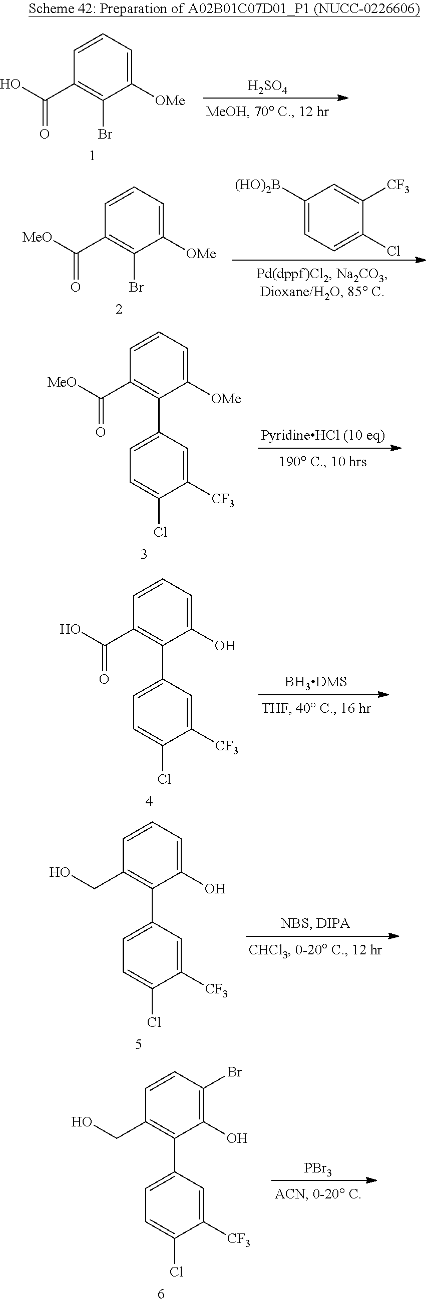

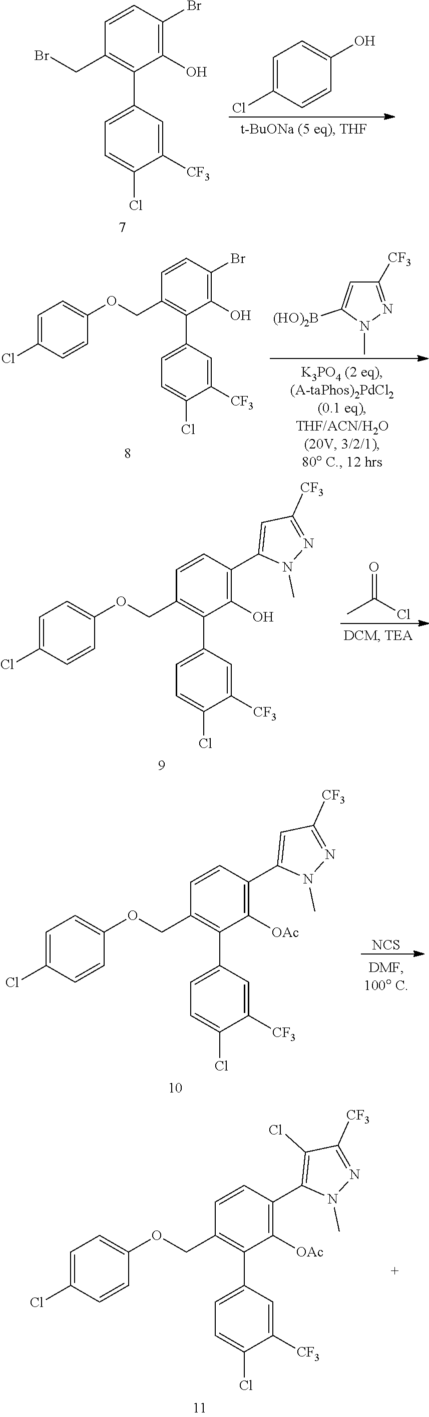

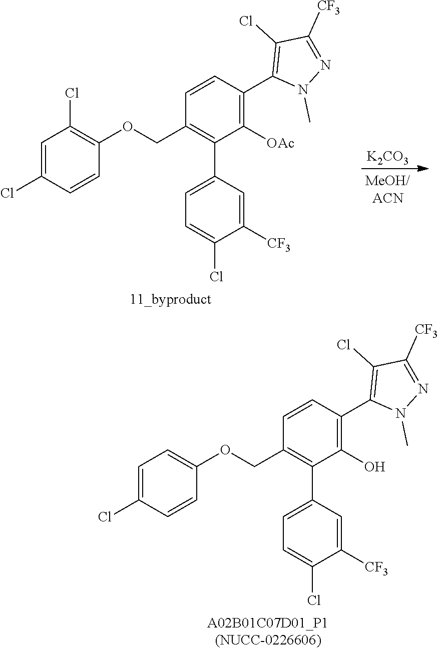

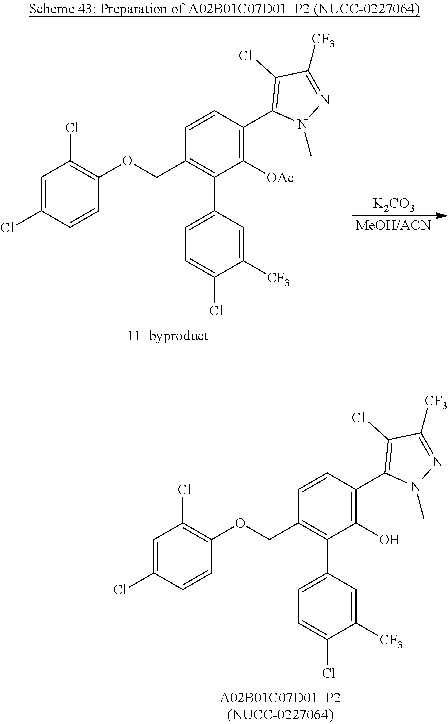

### Chemical Synthesis

### LCMS Method:

### 5_95AB_6min-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

### Experimentals for Largest Scale Run

### General Procedure for Preparation of Compound 2-ET52382-5

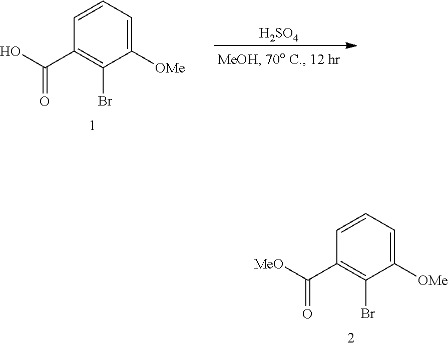 [SMILES: COc1cccc(C(=O)O)c1Br ; "H2SO4" ; COC(=O)c1cccc(OC)c1Br]

To a solution of Compound 1 (25 g, 108.20 mmol) in MeOH (250 ml) was added sulfuric acid (9.20 g, 93.80 mmol, 5 mL) dropwise at 20° C. The mixture was heated to 70° C. and stirred for 12 hrs. TLC (petroleum ether/ethyl acetate=3/1, Rf=0.25) showed the starting material was consumed and new spot generated. Two additional reaction were set up as detailed above and three reaction mixtures were combined. The reaction mixture was concentrated to give the crude product, which was diluted with water (500 mL), adjusted to pH=6-7 with K₂CO₃ and extracted with ethyl acetate (3×200 mL). The combined organic layers were washed with brine (100 mL) and dried over Na₂SO₄ and concentrated to give Compound 2 (76 g, 90.76% yield) as a white solid which was used directly for next step without further purification.

¹H NMR (400 MHz, CHLOROFORM-d) δ 3.93 (s, 3H), 3.94 (s, 3H), 7.02 (dd, J=8.00, 1.38 Hz, 1H), 7.26-7.28 (m, 1H), 7.30-7.36 (m, 1H)

### General Procedure for Preparation of Compound 3-ET52382-10

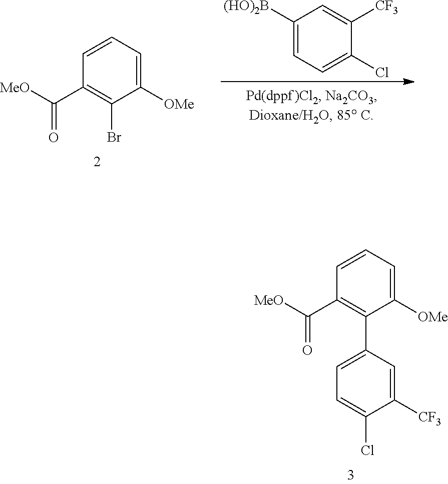 [SMILES: COC(=O)c1cccc(OC)c1Br ; OB(O)c1ccc(Cl)c(C(F)(F)F)c1 ; COC(=O)c1cccc(OC)c1-c1ccc(Cl)c(C(F)(F)F)c1]

To a solution of methyl 2-bromo-3-methoxy-benzoate (25 g, 102.01 mmol), [4-chloro-3-(trifluoromethyl)phenyl]boronic acid (27.47 g, 122.41 mmol) in dioxane (200 mL) and H₂O (100 mL) were added Na₂CO₃ (21.62 g, 204.02 mmol) and Pd(dppf)Cl₂·CH₂Cl₂ (4.17 g, 5.10 mmol) at 20° C. under nitrogen. The mixture was heated to 85° C. and stirred for 12 hrs. TLC (petroleum ether/ethyl acetate=10/1) showed the starting material (Rf=0.4) was consumed and new spot (Rf=0.45) generated. Two additional reaction were set up as detailed above and three reaction mixtures were combined. The reaction mixture was poured into water (800 mL) and extracted with ethyl acetate (200 ml×3). The organic layers were combined, washed with brine (2×20 ml), dried over Na₂SO₄, filtered and concentrated under reduced pressure to give a residue. The residue was purified by column chromatography on silica gel (eluting with petroleum ether/ethyl acetate=0/1 to 20/1) to give Compound 3 (85 g, 72.52% yield) as a white solid.

¹H NMR (400 MHz, CHLOROFORM-d) δ 3.62 (s, 3H), 3.77 (s, 3H), 7.14 (d, J=8.00 Hz, 1H), 7.36 (dd, J=8.19, 1.44 Hz, 1H), 7.41-7.47 (m, 1H), 7.48-7.54 (m, 2H), 7.57 (d, J=1.50 Hz, 1H)

### General Procedure for Preparation of Compound 4-ET52382-14

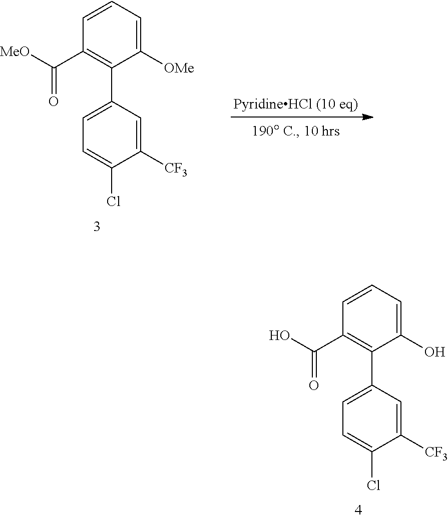 [SMILES: COC(=O)c1cccc(OC)c1-c1ccc(Cl)c(C(F)(F)F)c1 ; O=C(O)c1cccc(O)c1-c1ccc(Cl)c(C(F)(F)F)c1]

A mixture of methyl 2-[4-chloro-3-(trifluoromethyl)phenyl]-3-methoxy-benzoate (35 g, 101.53 mmol) and pyridine;hydrochloride (117.33 g, 1.02 mol) was heated to 190° C. and stirred for 10 hrs under nitrogen. TLC (petroleum ether/ethyl acetate=5/1) showed the starting material (Rf=0.6) was consumed and new spot (Rf=0.2) generated. Two additional reaction were set up as detailed above and three reaction mixtures were combined. The reaction mixture was diluted with water (1000 mL) and extracted with ethyl acetate (300 mL×3). The combined organic layer were washed with brine (100 mL×3) and dried over Na₂SO₄ and concentrated to give Compound 4 (90 g, 88.64% yield) as a white solid which was used directly for next step without further purification.

¹H NMR (400 MHz, DMSO-d6) δ 7.12 (d, J=7.75 Hz, 1H), 7.25-7.35 (m, 2H), 7.51 (br d, J=8.25 Hz, 1H), 7.60 (s, 1H), 7.70 (d, J=8.25 Hz, 1H), 9.88 (s, 1H), 12.70 (br s, 1H)

### General Procedure for Preparation of Compound 5-ET52382-16

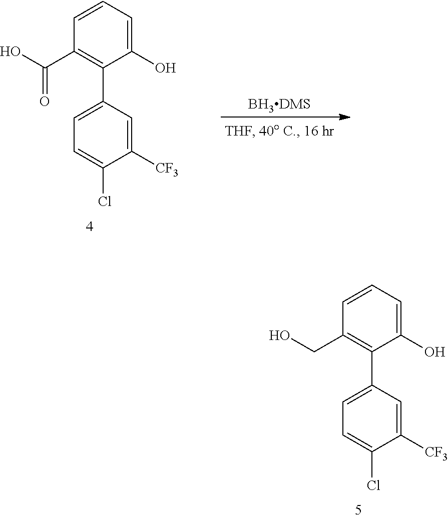 [SMILES: O=C(O)c1cccc(O)c1-c1ccc(Cl)c(C(F)(F)F)c1 ; OCc1cccc(O)c1-c1ccc(Cl)c(C(F)(F)F)c1]

To a solution of 2-[4-chloro-3-(trifluoromethyl)phenyl]-3-hydroxy-benzoic acid (40 g, 126.32 mmol) in THF (1000 mL) was added BH₃-Me₂S (10 M, 63.16 mL) dropwise at 0° C. The mixture was heated to 40° C. and stirred for 16 hrs. LCMS showed the starting material was consumed. The reaction mixture was cooled to 0° C., slowly quenched with methanol (200 mL) at 0° C. One additional reaction was set up as detailed above and two reaction mixtures were combined. The mixture was concentrated under reduced pressure to give a residue. The residue was purified by column chromatography on silica gel (eluting with petroleum ether/ethyl acetate=20/1 to 3/1) to give Compound 5 (72 g, 89.45% yield) as a white solid.

¹H NMR (400 MHz, DMSO-d6) δ 4.15 (d, J=5.25 Hz, 2H), 5.00 (t, J=5.32 Hz, 1H), 6.86 (d, J=8.00 Hz, 1H), 7.01 (d, J=7.50 Hz, 1H), 7.17-7.25 (m, 1H), 7.56 (dd, J=8.25, 1.75 Hz, 1H), 7.68 (d, J=1.75 Hz, 1H), 7.74 (d, J=8.25 Hz, 1H), 9.43 (s, 1H)

### General Procedure for Preparation of Compound 6-ET52382-20

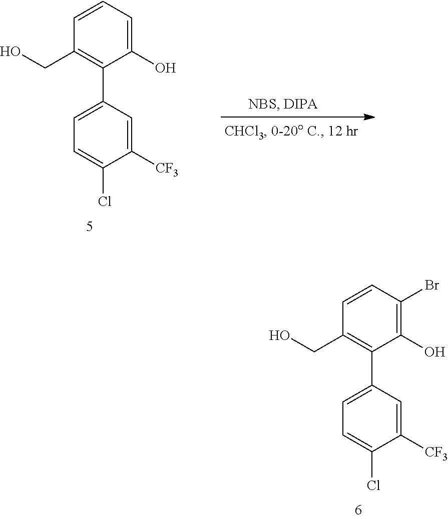 [SMILES: OCc1cccc(O)c1-c1ccc(Cl)c(C(F)(F)F)c1 ; OCc1ccc(Br)c(O)c1-c1ccc(Cl)c(C(F)(F)F)c1]

To a solution of 2-[4-chloro-3-(trifluoromethyl)phenyl]-3-(hydroxymethyl)phenol (30 g, 99.12 mmol), DIPA (30.09 g, 297.35 mmol, 42.02 mL) in CHCl₃ (700 mL) was added 1-bromopyrrolidine-2,5-dione (17.64 g, 99.12 mmol) portionwise at 0° C. The mixture was warmed up to 20° C. and stirred for 12 hrs. LCMS showed 16.1% of the starting material was remaining and 55% product (Rt=0.854 min) with desired Ms was detected. One additional reaction was set up as detailed above and two reaction mixtures were combined. The mixture was poured into water (1000 mL) and extracted with DCM (3×300 mL). The organic layers were combined, washed with brine (2×100 mL), dried over Na₂SO₄, filtered and concentrated under reduced pressure to give a residue. The residue was purified by column chromatography on silica gel (eluting with petroleum ether/ethyl acetate=50/1 to 10/1) to give Compound 6 (40 g, 50.24% yield) as a white solid.

¹H NMR (400 MHz, DMSO-d6) δ 4.08 (br d, J=3.26 Hz, 2H), 5.11 (br t, J=4.64 Hz, 1H), 7.03 (d, J=8.28 Hz, 1H), 7.51-7.60 (m, 2H), 7.69 (d, J=1.88 Hz, 1H), 7.78 (d, J=8.16 Hz, 1H), 8.93 (br s, 1H)

### General Procedure for Preparation of Compound 7-ET52382-23

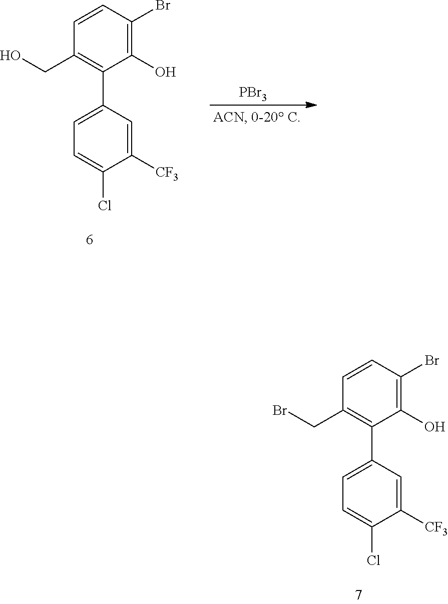 [SMILES: OCc1ccc(Br)c(O)c1-c1ccc(Cl)c(C(F)(F)F)c1 ; "PBr3" ; Oc1c(Br)ccc(CBr)c1-c1ccc(Cl)c(C(F)(F)F)c1]

To a solution of 6-bromo-2-[4-chloro-3-(trifluoromethyl)phenyl]-3-(hydroxymethyl)phenol (20 g, 52.41 mmol) in ACN (200 mL) was added PBr₃ (5.68 g, 20.97 mmol) dropwise at 0° C. The mixture was warmed up to 20° C. and stirred for 12 hrs. LCMS showed the starting material was consumed and 69.7% of product (Rt=0.975 min) with desired Ms was detected. The reaction mixture was poured into water (300 mL) and extracted with ethyl acetate (150 mL×3). The organic layers were combined, washed with brine (2×50 mL), dried over Na₂SO₄, filtered and concentrated under reduced pressure to give a residue. The residue was purified by column chromatography on silica gel (eluting with petroleum ether/ethyl acetate=1/0 to 50/1) to give Compound 7 (20 g, 77.26% yield) as a white solid.

¹H NMR (400 MHz, DMSO-d6) δ 4.32 (br d, J=5.50 Hz, 2H), 7.07 (d, J=8.38 Hz, 1H), 7.57-7.65 (m, 2H), 7.73 (d, J=1.38 Hz, 1H), 7.84 (d, J=8.25 Hz, 1H), 9.19 (s, 1H)

### General Procedure for Preparation of Compound 8-ET52382-25

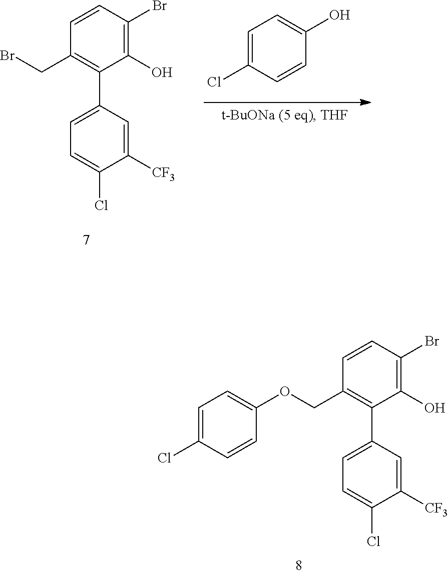 [SMILES: Oc1c(Br)ccc(CBr)c1-c1ccc(Cl)c(C(F)(F)F)c1 ; Oc1ccc(Cl)cc1 ; Oc1c(Br)ccc(COc2ccc(Cl)cc2)c1-c1ccc(Cl)c(C(F)(F)F)c1]

To a solution of 4-chlorophenol (28.92 g, 224.99 mmol, 22.08 mL) in a mixture solution of THF (100 mL) and DMF (100 mL) was added t-BuONa (21.62 g, 224.99 mmol). The reaction was stirred at 25° C. for 30 mins, Compound 7 (20 g, 45.00 mmol) in THF (50 mL) was added dropwise at 0° C. The reaction was stirred at 20° C. for 12 hrs. LCMS showed the starting material was consumed and 51.9% peak (Rt=1.019 min) with desired Ms was detected. TLC (petroleum ether/ethyl acetate=20/1, Rf=0.4) showed new spot generated. The reaction mixture was poured into water (300 mL) and extracted with ethyl acetate (3×100 mL). The organic layers were combined, washed with brine (2×50 mL), dried over Na₂SO₄, filtered and concentrated under reduced pressure to give a residue. The residue was purified by column chromatography on silica gel (eluting with petroleum ether/ethyl acetate=1/0 to 100/1) to give Compound 8 (20 g, 85.80% yield) as a white solid.

¹H NMR (400 MHz, DMSO-d6) δ 4.69 (br s, 2H), 6.75-6.85 (m, 2H), 7.06 (d, J=8.25 Hz, 1H), 7.21-7.30 (m, 2H), 7.55-7.65 (m, 2H), 7.67-7.76 (m, 2H), 9.16 (s, 1H)

### General Procedure for Preparation of Compound 9-ET52382-33

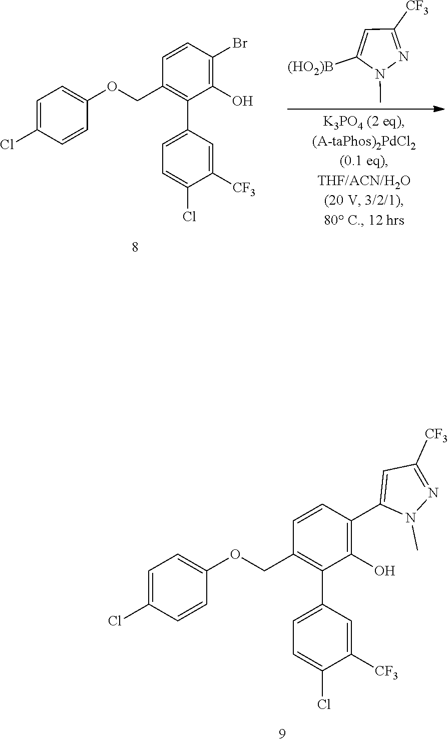

To a solution of Compound 8 (10 g, 20.32 mmol), [2-methyl-5-(trifluoromethyl) pyrazol-3-yl]-boronic acid (7.88 g, 40.64 mmol) in dioxane (80 mL), H₂O (40 mL) and ACN (80 mL) were added 4-ditert-butylphosphanyl-N,N-dimethyl-aniline;dichloropalladium (1.44 g, 2.03 mmol) and K₃PO₄ (12.94 g, 60.96 mmol) under nitrogen at 20° C. The mixture was heated to 80° C. and stirred for 12 hrs. LCMS showed the starting material was consumed and 73.3% peak (Rt=1.041 min) with desired Ms was detected. TLC (petroleum ether/ethyl acetate=5/1, Rf=0.5) showed new spot generated. The reaction mixture was poured into water (200 mL) and extracted with ethyl acetate (60) ml×3). The organic layers were combined, washed with brine (2×50 mL), dried over Na₂SO₄, filtered and concentrated under reduced pressure to give a residue. The residue was purified by column chromatography on silica gel (eluting with petroleum ether/ethyl acetate=1/0 to 10/1) to give Compound 9 (9 g, 71.02% yield) as a white solid.

¹H NMR (400 MHz, DMSO-de) 8 3.78 (s, 3H), 4.61-4.91 (m, 2H), 6.80 (s, 1H), 6.82-6.91 (m, 2H), 7.21-7.31 (m, 3H), 7.37 (d, J=7.88 Hz, 1H), 7.65 (dd, J=8.19, 1.69 Hz, 1H), 7.71-7.80 (m, 2H), 9.01 (s, 1H)

### General Procedure for Preparation of Compound 10-ET52382-58

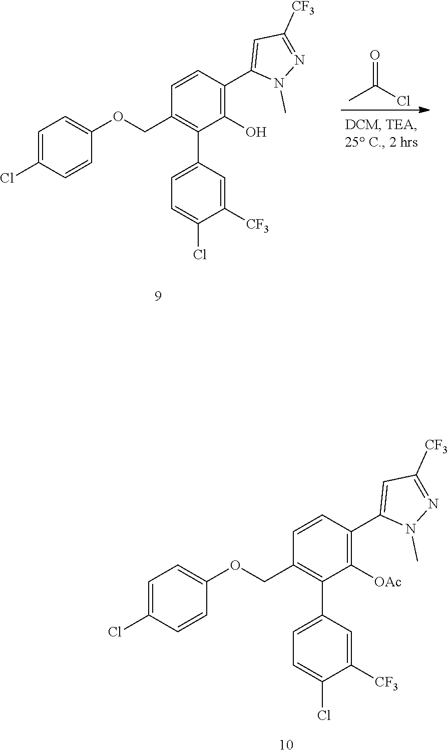

To a solution of Compound 9 (10 g, 17.82 mmol). TEA (2.70 g, 26.72 mmol, 3.72 mL) in DCM (150 mL) was added acetyl chloride (1.68 g, 21.38 mmol, 1.53 mL) dropwise at ° C. The mixture was warmed up to 20° C. and stirred for 2 hrs. LCMS showed the starting material was consumed and 93.4% peak (Rt=0.916 min) with desired Ms was detected. TLC (petroleum ether/ethyl acetate=5/1, Rf=0.4) showed new spot generated. The reaction mixture was poured into water (200 mL) and extracted with DCM (60 mL×3). The organic layers were combined, washed with brine (2×20 mL), dried over Na₂SO₄, filtered and concentrated under reduced pressure to give a residue. The residue was purified by column chromatography on silica gel (eluting with petroleum ether/ethyl acetate=50/1 to 10/1) to give Compound 10 (10.2 g, 85.40% yield) as a white solid.

¹H NMR (400 MHz, DMSO-d6) δ 1.71 (s, 3H), 3.82 (s, 3H), 4.80-4.99 (m, 2H), 6.78 (s, 1H), 6.84-6.91 (m, 2H), 7.24-7.32 (m, 2H), 7.61 (br d, J=7.88 Hz, 1H), 7.72 (br s, 1H), 7.75 (s, 2H), 7.79 (d, J=8.25 Hz, 1H)

### General Procedure for Preparation of Compound 11-ET52382-61

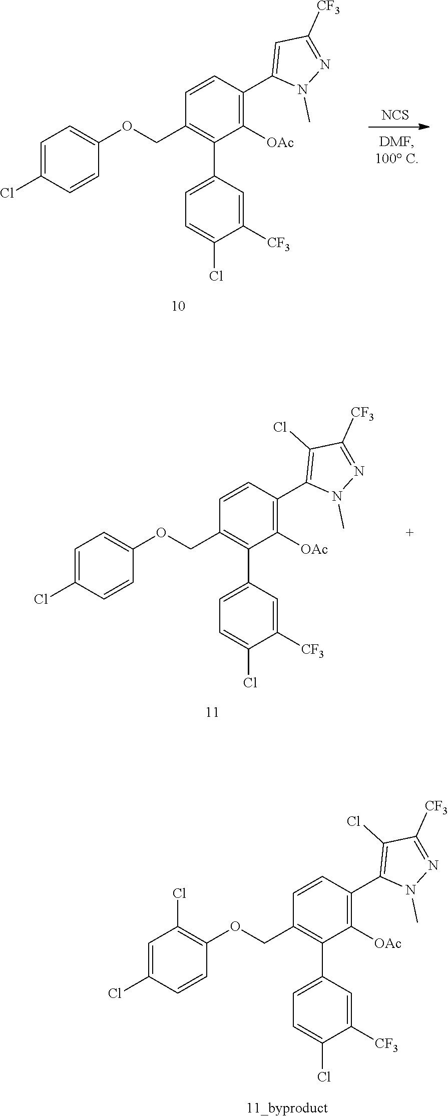 [SMILES: CC(=O)Oc1c(-c2cc(C(F)(F)F)nn2C)ccc(COc2ccc(Cl)cc2)c1-c1ccc(Cl)c(C(F)(F)F)c1 ; "NCS" ; CC(=O)Oc1c(-c2c(Cl)c(C(F)(F)F)nn2C)ccc(COc2ccc(Cl)cc2)c1-c1ccc(Cl)c(C(F)(F)F)c1 ; CC(=O)Oc1c(-c2c(Cl)c(C(F)(F)F)nn2C)ccc(COc2ccc(Cl)cc2Cl)c1-c1ccc(Cl)c(C(F)(F)F)c1]

To a solution of Compound 10 (10 g, 16.57 mmol) in DMF (100 mL) was added 1-chloropyrrolidine-2,5-dione (8.85 g, 66.30 mmol) at 20° C. The mixture was heated to 100° C. and stirred for 12 hrs. LCMS and HPLC showed 2.5% of the starting material was remaining and 67.3% product with desired Ms was detected. The reaction mixture was poured into water (600 mL) and extracted with ethyl acetate (3×200 mL). The organic layers were combined, washed with brine (2×50 mL), dried over Na₂SO₄, filtered and concentrated under reduced pressure to give a residue. The residue was purified by prep-HPLC to give Compound 11 (4.5 g, 42.36% yield) and Compound 11_byproduct (0.50 g, 4.44% yield) both as a white solid.

Compound 11: ¹H NMR (400 MHz, DMSO-d₆) δ 1.65 (s, 3H), 3.73 (s, 3H), 4.72-4.97 (m, 2H), 6.82 (br d, J=8.82 Hz, 2H), 7.22 (br d, J=8.82 Hz, 2H), 7.47-7.70 (m, 2H), 7.70-7.80 (m, 3H)

Compound 11_byproduct: ¹H NMR (400 MHz, DMSO-d₆) δ 1.72 (s, 3H), 3.80 (s, 3H), 4.94-5.15 (m, 2H), 7.09 (br d, J=8.88 Hz, 1H), 7.33 (dd, J=8.88, 2.50 Hz, 1H), 7.56 (d, J=2.38 Hz, 1H), 7.59-7.77 (m, 2H), 7.78-7.90 (m, 3H)

### Separation Method of Prep-HPLC:

Instrument: Shimadzu LC-8A preparative HPLC
Column: Phenomenex luna C18 (250*70 mm, 15 um)
Mobile phase: A for H₂O) (0.09% TFA) and B for CAN
Gradient: B from 73% to 90% in 20 min
Flow rate: 130 mL/min
Wavelength: 220&254 nm

### General Procedure for Preparation of A02B01C07D01_P1-ET52382-63

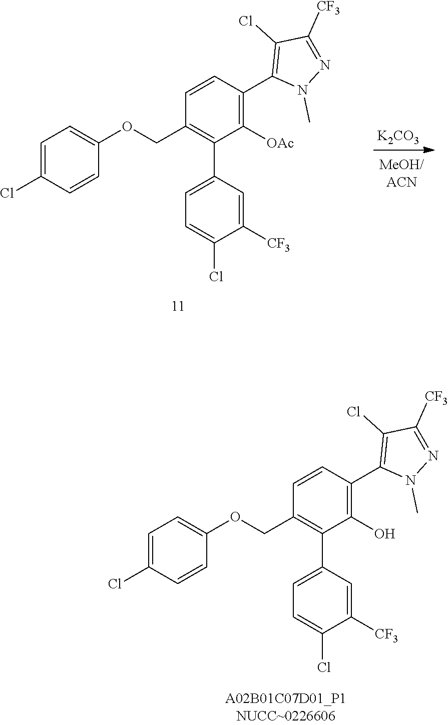

To a solution of Compound 11 (6.5 g, 10.19 mmol) in MeOH (100 mL) and ACN (100 mL) was added K₂CO₃ (4.23 g, 30.57 mmol) at 20° C. The mixture was stirred for 12 hrs. LCMS showed the starting material was consumed and 96.6% peak (Rt=0.941 min) with desired Ms was detected. The reaction mixture was concentrated under reduced pressure to remove methanol and acetonitrile. Then water (200 mL) was added and extracted with ethyl acetate (3×60 mL). The organic layers were combined, washed with brine (2×20 mL), dried over Na₂SO₄, filtered and concentrated under reduced pressure to give a residue. The residue was co-evaporation with DCM (60 mL) three times to give A02B01C07D01_P1 (5.2 g, 84.70% yield) as a white solid.

¹H NMR (400 MHz, DMSO-d6) δ 3.77 (s, 3H), 4.78 (br s, 2H), 6.82-6.91 (m, 2H), 7.24-7.32 (m, 3H), 7.40 (d, J=8.00 Hz, 1H), 7.64-7.70 (m, 1H), 7.72-7.76 (m, 1H), 7.78 (s, 1H), 9.24 (s, 1H)

$$LCMS ( ESI + ) : RT = 596.9 ( M + H ) + RT : 3.26 min$$

### 30_90AB_6min-220-254-ELSD:

30-90AB_6min-220-254-ELSD: LC/MS (The gradient was 30% B in 0.40 min and 30-90% B in 2.60 min, hold on 90% B in 1.00 min, and then 90-30% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Kinetex C18 2.1*50 mm, 5 um. Detection methods are diode array (DAD) and evaporative light scattering detection (ELSD). MS mode was positive electrospray ionization. MS range was 100-1000.

### General Procedure for Preparation of A02B01C07D01_P2-ET52382-64

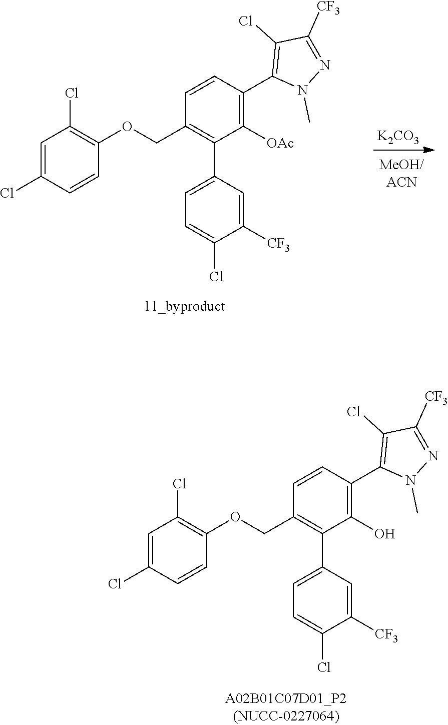

To a solution of Compound 11_byproduct (0.5 g, 743.79 μmol) in MeOH (20 mL) and ACN (20 mL) was added K₂CO₃ (308.39 mg, 2.23 mmol) at 20° C. The mixture was stirred for 12 hrs. LCMS showed the starting material was consumed and 98.9% peak (Rt=0.957 min) with desired Ms was detected. The reaction mixture was concentrated under reduced pressure to remove methanol and acetonitrile. Then water (60 mL) was added and extracted with ethyl acetate (20 mL×3). The organic layers were combined, washed with brine (2×10 ml), dried over Na₂SO₄, filtered and concentrated under reduced pressure to give a residue. The residue was co-evaporation with DCM (30 mL) three times to give A02B01C07D01_P2 (424 mg, 89.46% yield) as a white solid.

¹H NMR (400 MHz, DMSO-d₆) δ 3.78 (s, 3H), 4.90 (br s, 2H), 7.05 (d, J=8.88 Hz, 1H), 7.26-7.37 (m, 2H), 7.39-7.47 (m, 1H), 7.55 (d, J=2.25 Hz, 1H), 7.64-7.71 (m, 1H), 7.72-7.82 (m, 2H), 9.27 (s, 1H)

$$LCMS ( ESI + ) : m / z 630.9 ( M + H ) + , RT : 2.67 min$$

50-100AB_6min-220-254-ELSD: LC/MS (The gradient was 50% B in 0.40 min and 50-100% B in 2.60 min, hold on 100% B in 1.00 min, and then 100-50% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Kinetex C18 2.1*50 mm, Sum. Detection methods are diode array (DAD), and evaporative light scattering detection (ELSD). MS mode was positive electrospray ionization. MS range was 100-1000.

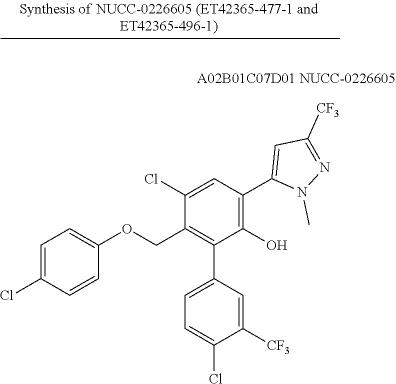 [SMILES: Cn1nc(C(F)(F)F)cc1-c1cc(Cl)c(COc2ccc(Cl)cc2)c(-c2ccc(Cl)c(C(F)(F)F)c2)c1O]

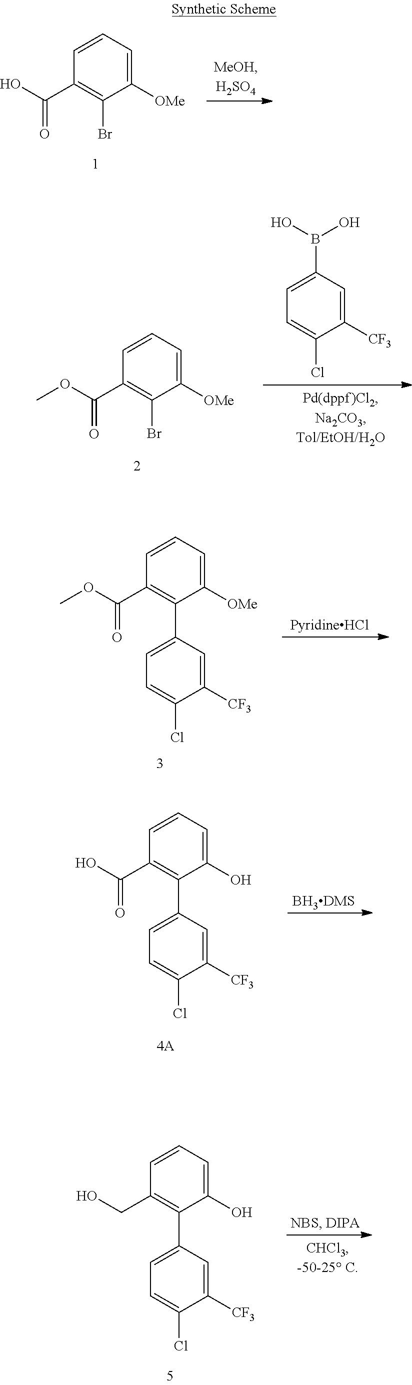

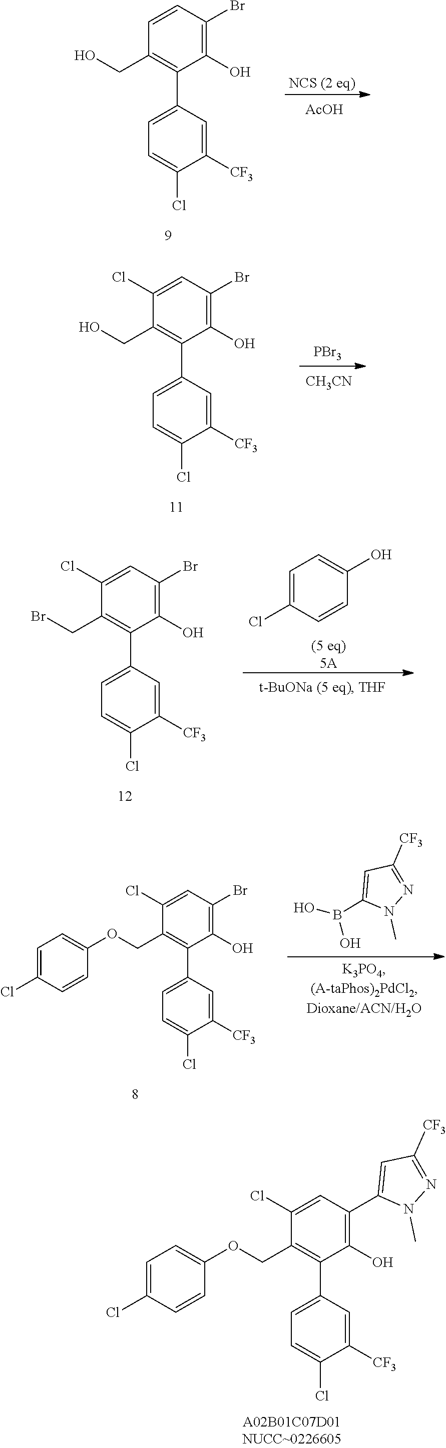

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% trifluoroacetic acid in water, mobile phase B was 0.018% trifluoroacetic acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

### Experimental for Largest Scale Run

### General Procedure for Preparation of Compound 2-ET42365-393

The reactions were conducted in parallel but combined for purification.

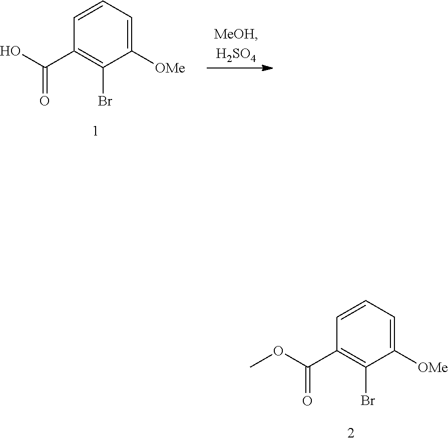

To a solution of Compound 1 (10 g, 43.28 mmol, 1 eq) in MeOH (100 mL) was added H₂SO₄ (3.68 g, 37.52 mmol, 2 mL), the reaction was stirred at 70° C. for 12 hrs. The reaction was cooled to room temperature. LCMS showed the starting material was consumed and a new peak with desired product Ms was detected. One additional reaction was set up as detailed above and both two reaction mixtures were combined. The combined reaction mixture was concentrated to give a residue, which was diluted with water (100 mL), adjusted to pH=6˜7 with aq K₂CO₃ solution and extracted with ethyl acetate (3×100 mL). The combined organic layers were washed with brine (200 mL), dried over Na₂SO₄ and concentrated to give Compound 2 (21 g, yield 94.04%) as a yellow solid, which was used to the next step directly without further purification.

¹H NMR (ET42365-393-P1A, 400 MHz, CHLOROFORM-d) δ 3.94 (s, 3H), 3.95 (s, 3H), 7.03 (dd, J=8.03, 1.51 Hz, 1H), 7.26-7.30 (m, 1H), 7.31-7.37 (m, 1H)

### General Procedure for Preparation of Compound 3-ET42365-403

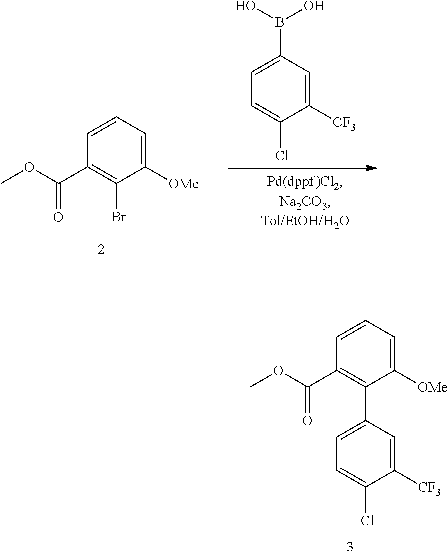 [SMILES: COC(=O)c1cccc(OC)c1Br ; OB(O)c1ccc(Cl)c(C(F)(F)F)c1 ; "Pd(dppf)Cl2,Na2CO3,Tol/EtOH/H2O" ; COC(=O)c1cccc(OC)c1-c1ccc(Cl)c(C(F)(F)F)c1]

To a solution of Compound 2 (14 g, 57.13 mmol, 1 eq), [4-chloro-3-(trifluoromethyl)phenyl]-boronic acid (15.38 g, 68.55 mmol, 1.2 eq) and Na₂CO₃ (12.11 g, 114.25 mmol, 2 eq) in a mixture solution of toluene (500 mL), EtOH (100 ml) and H₂O (25 mL) was added Pd(dppf)Cl₂·CH₂Cl₂ (2.33 g, 2.86 mmol, 0.05 eq) under nitrogen, the reaction was stirred at 85° C. for 12 hrs. LCMS showed the starting material was consumed and a new peak with desired product Ms was detected. The reaction was filtered and the filtrate was concentrated to give the crude product. The crude product was purified by column chromatography on silica gel (eluted with petroleum ether/ethyl acetate=30/1 to 4/1) to give Compound 3 (13 g, yield 62.72%) as a yellow solid.

¹H NMR (ET42365-403-P1A, 400 MHz, CHLOROFORM-d) δ 3.62 (s, 3H), 3.77 (s, 3H), 7.14 (dd, J=8.07, 1.06 Hz, 1H), 7.36 (dd, J=8.19, 1.81 Hz, 1H), 7.41-7.47 (m, 1H), 7.47-7.54 (m, 2H), 7.57 (d, J=2.00 Hz, 1H)

### General Procedure for Preparation of Compound 4A-ET42365-431

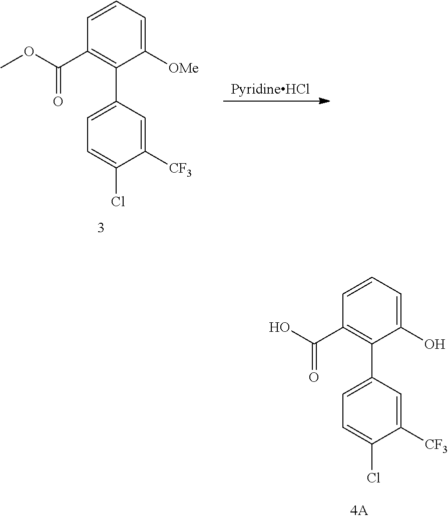 [SMILES: COC(=O)c1cccc(OC)c1-c1ccc(Cl)c(C(F)(F)F)c1 ; "Pyridine·HCl" ; O=C(O)c1cccc(O)c1-c1ccc(Cl)c(C(F)(F)F)c1]

A mixture of Compound 3 (10 g, 29.01 mmol, 1 eq) in pyridine hydrochloride (50.29 g, 435.15 mmol, 15 eq) was stirred at 190° C. for 6 hrs. The reaction was cooled to room temperature, LCMS showed the starting material was consumed and a new peak with desired product Ms was detected. The reaction mixture was diluted with water (200 mL) and was extracted with ethyl acetate (3×100 mL). The combined organic layer were washed with brine (150 mL), dried over Na₂SO₄ and concentrated to give Compound 4A (8 g, yield 87.09%) as a yellow solid, which was used to the next step directly without further purification.

¹H NMR (ET42365-431-P1A, 400 MHz, CHLOROFORM-d) δ 7.21 (dd, J=8.19, 1.06 Hz, 1H), 7.38-7.45 (m, 2H), 7.58-7.65 (m, 2H), 7.69 (dd, J=7.82, 1.06 Hz, 1H)

### General Procedure for Preparation of Compound 5-ET42365-438

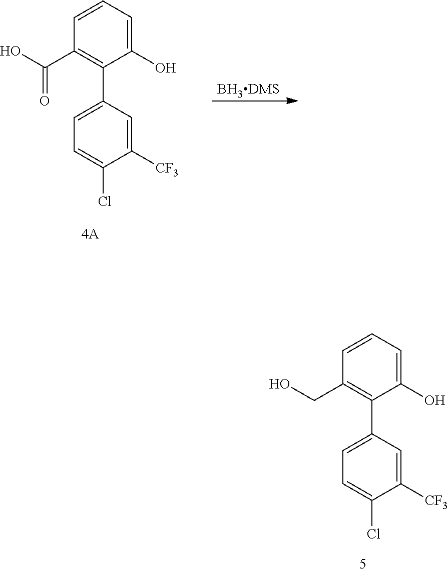 [SMILES: O=C(O)c1cccc(O)c1-c1ccc(Cl)c(C(F)(F)F)c1 ; "BH3·DMS" ; OCc1cccc(O)c1-c1ccc(Cl)c(C(F)(F)F)c1]

To a solution of Compound 4A (8 g, 25.26 mmol, 1 eq) in THF (300 mL) was added BH₃-Me₂S (10 M, 12.63 mL, 5 eq) dropwise at 0° C., the reaction was stirred at 25° C. for 12 hrs. LCMS showed the starting material was consumed and a new peak with desired product Ms was detected. The reaction mixture was cooled to 0° C., slowly quenched with methanol (100 mL) and concentrated in vacuo to give crude product. The crude product was purified by column chromatography on silica gel (eluted with petroleum ether/ethyl acetate=10/1 to 2/1) to give Compound 5 (6.6 g, yield 86.31%) as a white solid.

¹H NMR (ET42365-438-P1A, 400 MHz, CHLOROFORM-d) & 4.39 (s, 2H), 4.72 (s, 1H), 6.93 (dd, J=8.13, 0.75 Hz, 1H), 7.15 (d, J=7.50 Hz, 1H), 7.30-7.37 (m, 1H), 7.49 (dd, J=8.13, 1.88 Hz, 1H), 7.64 (d, J=8.25 Hz, 1H), 7.70 (d, J=1.88 Hz, 1H)

### General Procedure for Preparation of Compound 9-ET42365-452

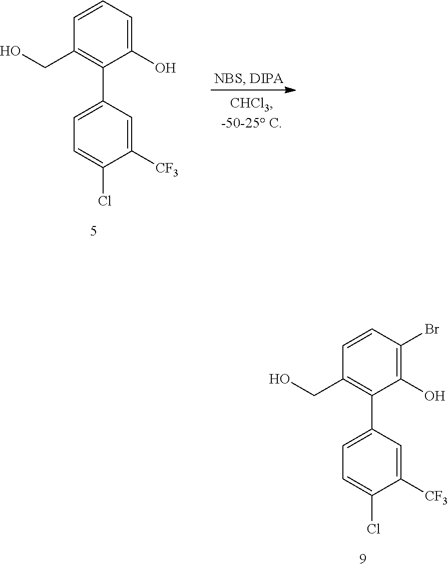

To a solution of Compound 5 (5 g, 16.52 mmol, 1 eq) and DIPA (5.01 g, 49.56 mmol, 7.00 mL, 3 eq) in CHCl₃ (125 mL) was added NBS (2.79 g, 15.69 mmol, 0.95 eq) in portions at −50° C., the reaction was slowly warmed to 25° C. and stirred for 12 hrs. LCMS showed about 3% of the starting material was remaining and a new peak with desired product Ms was detected. One additional reaction was set up as detailed above and both two reaction mixtures were combined. The combined reaction mixture was diluted with water (300 mL) and was extracted with DCM (3×150 mL). The combined organic layer were washed with brine (250 mL) and dried over Na₂SO₄ and concentrated to give crude product. The crude product was purified by Prep-HPLC (column: Agela DuraShell C18 250*70 mm*10 um; mobile phase: [water (TFA)-ACN]; B %: 38%-68%, 20 min) to give Compound 9 (8 g, yield 63.46%) as a white solid.

¹H NMR (ET42365-452-P1A, 400 MHz, CHLOROFORM-d) δ 4.38 (s, 2H), 5.54 (br s, 1H), 7.09 (d, J=8.38 Hz, 1H), 7.44 (dd, J=8.13, 2.00 Hz, 1H), 7.56 (d, J=8.38 Hz, 1H), 7.60 (d, J=8.13 Hz, 1H), 7.66 (d, J=2.00 Hz, 1H)

### General Procedure for Preparation of Compound 11-ET42365-464

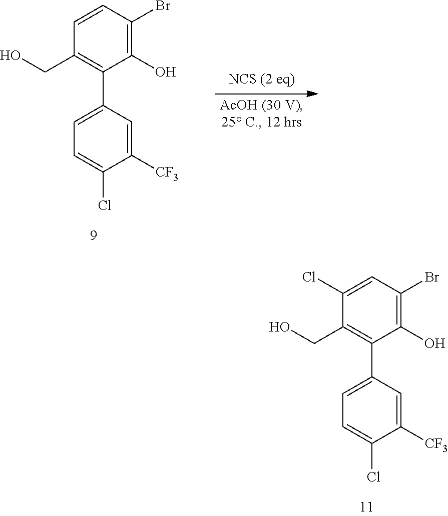 [SMILES: OCc1ccc(Br)c(O)c1-c1ccc(Cl)c(C(F)(F)F)c1 ; "NCS (2 eq)" ; "AcOH (30 V),25° C., 12 hrs" ; OCc1c(Cl)cc(Br)c(O)c1-c1ccc(Cl)c(C(F)(F)F)c1]

To a solution of Compound 9 (8 g, 20.97 mmol, 1 eq) in AcOH (160 mL) was added NCS (5.60 g, 41.93 mmol, 2 eq), the reaction was stirred at 40° C. for 12 hrs. LCMS showed the starting material was consumed and a new peak with desired product Ms was detected. The reaction mixture was concentrated to give a residue, diluted with water (200 mL) and was extracted with ethyl acetate (3×100 mL). The combined organic layer were washed with brine (150 mL), dried over Na₂SO₄ and concentrated to give crude product. The crude product was purified by column chromatography on silica gel (eluted with petroleum ether/ethyl acetate=50/1 to 10/1) to give Compound 11 (6.5 g, yield 74.52%) as a yellow solid.

¹H NMR (ET42365-464-P1A, 400 MHz, CHLOROFORM-d) δ 1.97 (br s, 1H), 4.33-4.43 (m, 1H), 4.45-4.54 (m, 1H), 5.53 (s, 1H), 7.50 (dd, J=8.16, 2.01 Hz, 1H), 7.60-7.65 (m, 2H), 7.71 (d, J=1.88 Hz, 1H)

### General Procedure for Preparation of Compound 12-ET42365-466

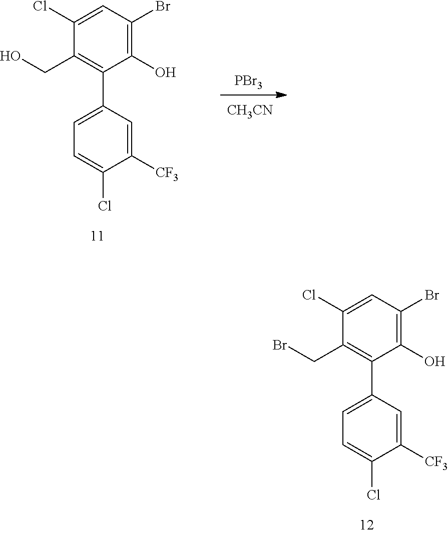 [SMILES: OCc1c(Cl)cc(Br)c(O)c1-c1ccc(Cl)c(C(F)(F)F)c1 ; "PBr3" ; "CH3CN" ; Oc1c(Br)cc(Cl)c(CBr)c1-c1ccc(Cl)c(C(F)(F)F)c1]

To a solution of Compound 11 (6.5 g, 15.62 mmol, 1 eq) in CH₃CN (160 mL) was added PBr₃ (2.54 g, 9.37 mmol, 0.6 eq) dropwise at 0° C., the reaction was stirred at 25° C. for 12 hrs. LCMS showed the starting material was consumed and a new peak with desired product Ms was detected. The reaction was quenched with water (200 mL) and extracted with ethyl acetate (3×100 mL). The combined organic layer were washed with brine (150 mL), dried over Na₂SO₄ and concentrated to give Compound 12 (6.5 g, yield 86.87%) as a yellow oil, which was used to the next step directly without further purification.

¹H NMR (ET42365-466-P1A, 400 MHz, CHLOROFORM-d) δ 4.17-4.23 (m, 1H), 4.24-4.31 (m, 1H), 5.46 (br s, 1H), 7.50-7.58 (m, 1H), 7.61-7.69 (m, 2H), 7.73 (d, J=1.88 Hz, 1H)

### General Procedure for Preparation of Compound 8-ET42365-468

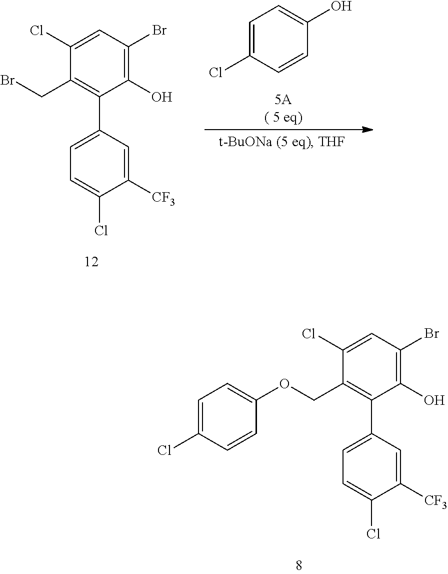 [SMILES: Oc1c(Br)cc(Cl)c(CBr)c1-c1ccc(Cl)c(C(F)(F)F)c1 ; Oc1ccc(Cl)cc1 ; Oc1c(Br)cc(Cl)c(COc2ccc(Cl)cc2)c1-c1ccc(Cl)c(C(F)(F)F)c1]

To a solution of Compound 5A (8.72 g, 67.86 mmol, 6.66 mL, 5 eq) in a mixture solution of THF (75 mL) and DMF (75 mL) was added NaOt-Bu (6.52 g, 67.86 mmol, 5 eq), the reaction was stirred at 25° C. for 30 mins, then Compound 12 (6.5 g, 13.57 mmol, 1 eq) in THF (25 mL) was added dropwise, the reaction was stirred at 30° C. for 12 hrs. LCMS showed the starting material was consumed and a new peak with desired product Ms was detected. The reaction mixture was diluted with water (300 mL) and was extracted with ethyl acetate (3×100 mL). The combined organic layer were washed with brine (150 mL), dried over Na₂SO₄ and concentrated to give crude product. The crude product was purified by column chromatography on silica gel (eluted with petroleum ether/ethyl acetate=50/1 to 20/1) to give Compound 8 (5 g, yield 69.96%) as a white solid.

¹H NMR (ET42365-468-P1A, 400 MHz, CHLOROFORM-d) δ 4.65-4.78 (m, 2H), 5.55 (s, 1H), 6.72-6.80 (m, 2H), 7.18-7.25 (m, 2H), 7.43-7.49 (m, 1H), 7.51-7.55 (m, 1H), 7.67-7.72 (m, 2H)

### General Procedure for Preparation of A02B01C07D01-ET42365-477

The reactions were conducted in parallel but combined for purification.

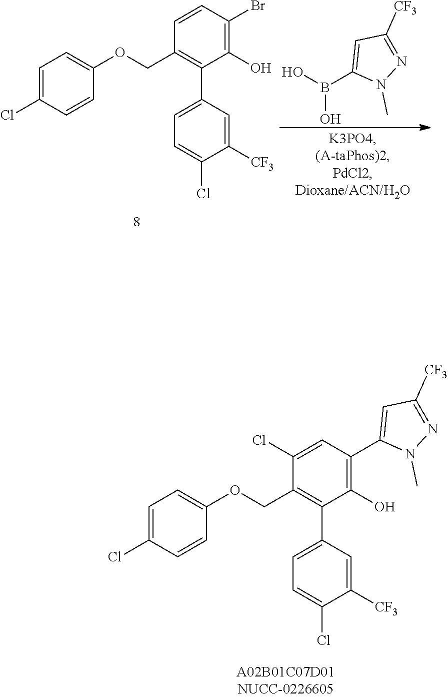

To a solution of Compound 8 (300 mg, 569.74 μmol, 1 eq), [2-methyl-5-(trifluoromethyl)-pyrazol-3-yl]boronic acid (220.97 mg, 1.14 mmol, 2 eq) and K₃PO₄ (241.87 mg, 1.14 mmol, 2 eq) in a mixture solution of CH₃CN (6 mL), dioxane (6 mL) and H₂O 13 mL) was added 4-ditert-butylphosphanyl-N,N-dimethyl-aniline;dichloropalladium (40.34 mg, 56.97 μmol, 0.1 eq) under nitrogen, the reaction was stirred at 85° C. for 12 hrs. LCMS showed the starting material was consumed and a new peak with desired product Ms was detected. Additional four reactions were set up as described above and combined for purification. The combined reaction was filtered and the filtrate was concentrated to give the crude product. The crude product was purified by column chromatography on silica gel (eluted with petroleum ether/ethyl acetate=50/1 to 10/1) to give crude product, which was triturated with CH₃CN (5 mL) to afford A02B01C07D01 (962 mg, yield 56.68%) as a white solid.

¹H NMR (ET42365-477-P1D, 400 MHz, CHLOROFORM-d) δ 3.90 (s, 3H), 4.72-4.78 (m, 1H), 4.79-4.85 (m, 1H), 5.02 (s, 1H), 6.65 (s, 1H), 6.74-6.81 (m, 2H), 7.20-7.26 (m, 2H), 7.45 (s, 1H), 7.49-7.55 (m, 1H), 7.59-7.65 (m, 1H), 7.76 (d, J=1.88 Hz, 1H)

$$LCMS ( ESI + ) : RT : 3.415 min , m / z 596.9 {( M + H )}^{+} ,$$

5_95AB_6min-220-254-ELSD: LC/MS (The gradient was 5% B in 0.40 min and 5-95% B at 0 40-3.00 min. hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (Sum particles). Detection methods are diode array (DAD) and evaporative light scattering (ELSD) detection as well as positive electrospray ionization. MS range was 100-1000.

### General Procedure for Preparation of A02B01C07D01-ET42365-496

The reactions were conducted in parallel but combined for purification.

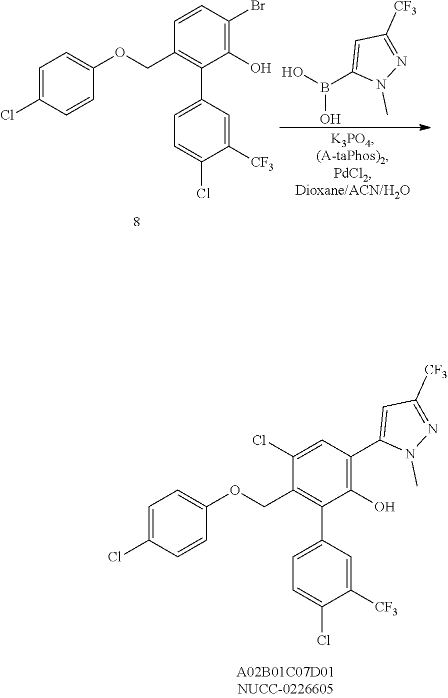

To a solution of Compound 8 (300 mg, 569.74 μmol, 1 eg), [2-methyl-5-(trifluoromethyl) pyrazol-3-yl]boronic acid (331.45 mg, 1.71 mmol, 3 eq) and K₃PO₄ (241.87 mg, 1.14 mmol, 2 eq) in a mixture solution of CH₃CN (6 mL), dioxane (6 mL) and H₂O (3 mL) was added 4-ditert-butylphosphanyl-N,N-dimethyl-aniline; dichloropalladium (40.34 mg, 56.97 μmol, 40.34 μL, 0.1 eq) under nitrogen, the reaction was stirred at 82° C. for 12 hrs. TLC (petroleum ether/ethyl acetate=5/1) showed the starting material was consumed and a new spot was generated. Additional nine reactions were set up as described above and all ten combined for purification. The combined reaction was filtered and the filtrate was concentrated to give the crude product. The crude product was purified by column chromatography on silica gel (eluted with petroleum ether/ethyl acetate=50/1 to 10/1) to give crude product, which was triturated with CH₃CN (10 mL) to afford A02B01C07D01 (1.02 g, yield 33.39%) as a white solid.

¹H NMR (ET42365-496-P1A, 400 MHz, CHLOROFORM-d) δ 3.90 (s, 3H), 4.72-4.78 (m, 1H), 4.79-4.86 (m, 1H), 5.01 (s, 1H), 6.65 (s, 1H), 6.74-6.82 (m, 2H), 7.19-7.26 (m, 2H), 7.45 (s, 1H), 7.52 (dd, J=8.19, 1.94 Hz, 1H), 7.62 (d, J=8.13 Hz, 1H), 7.76 (d, J=1.88 Hz, 1H)

$$LCMS ( ESI + ) : RT = 3.408 min , m / z 594.9 {( M + H )}^{+} .$$

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetes C18 50*2.1 mm column (5 um particles). Detection methods are diode array (DAD) and evaporative light scattering (ELSD) detection as well as positive electrospray ionization. MS range was 100-1000.

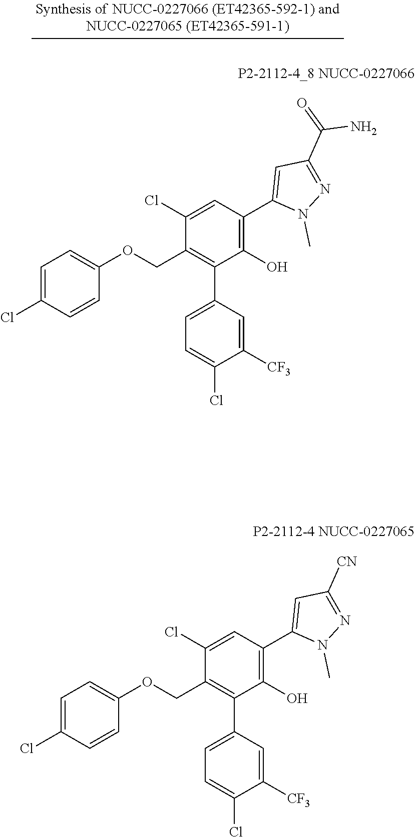 [SMILES: Cn1nc(C(N)=O)cc1-c1cc(Cl)c(COc2ccc(Cl)cc2)c(-c2ccc(Cl)c(C(F)(F)F)c2)c1O ; Cn1nc(C#N)cc1-c1cc(Cl)c(COc2ccc(Cl)cc2)c(-c2ccc(Cl)c(C(F)(F)F)c2)c1O]

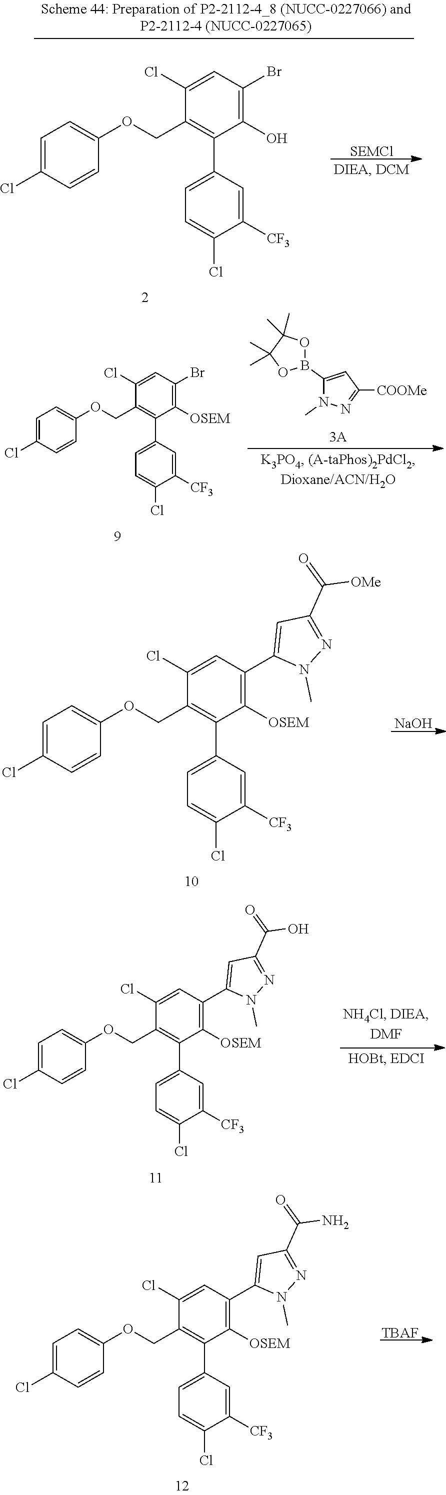

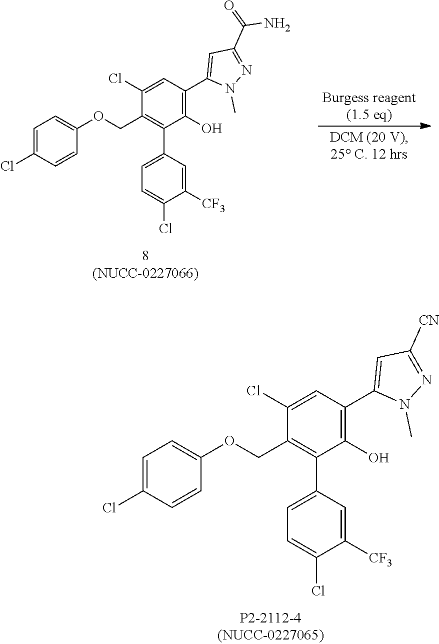 [SMILES: Cn1nc(C(N)=O)cc1-c1cc(Cl)c(COc2ccc(Cl)cc2)c(-c2ccc(Cl)c(C(F)(F)F)c2)c1O ; Cn1nc(C#N)cc1-c1cc(Cl)c(COc2ccc(Cl)cc2)c(-c2ccc(Cl)c(C(F)(F)F)c2)c1O]

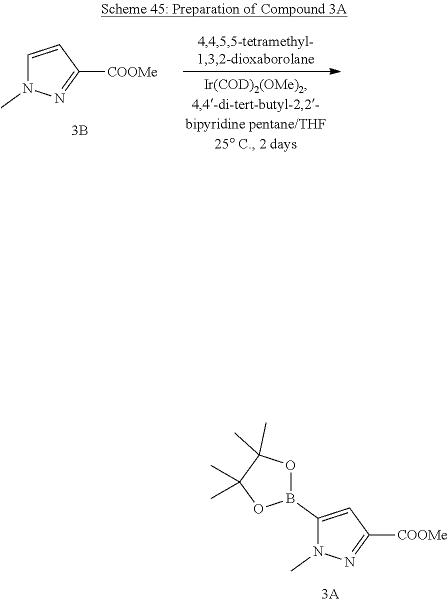

### Chemical Synthesis

5-95AB_2_min: LC/MS (The column used for chromatography was a Kinetex EVO C18 2.1*30 mm, Sum. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

50-100CD_2_min: LC/MS (The column used for chromatography was Xbridge C18 2.1*50 mm, 5 um. Detection methods are diode array (DAD). MS mode was positive electrospray ionization. MS range was 100-1000. Mobile phase A was 10 mM ammonium bicarbonate in water, and mobile phase B was HPLC grade acetonitrile. The gradient was 50-100% B in 1.50 min 0.50% B in 0.01 min, 50-100% B (0.01-0.80 min) with a hold at 100% B for 0.40 min, 100-50% B (1.20-1.21 min) with a hold at 50% B for 0.29 min. The flow rate was 1.5 mL/min.

50_100AB_6min-220-254-ELSD: LC/MS (The gradient was 50% B in 0.40 min and 50-100% B at 0.40-3.00 min, hold on 100% B for 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1000.

### Experimentals for Largest Scale Run

### General Procedure for Preparation of Compound 9-ET43365-569

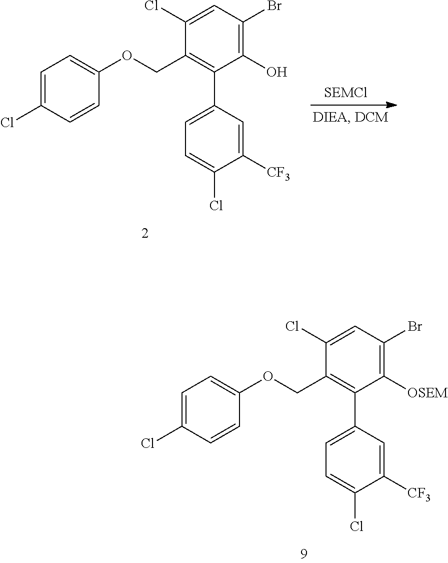

To a solution of Compound 2 (0.5 g, 949.56 μmol, 1 eq) and DIEA (368.16 mg, 2.85 mmol, 496.18 μL, 3 eq) in DCM (10 mL) was added SEM-Cl (316.62 mg, 1.90 mmol, 336.12 μL, 2 eq) dropwise at 0° C., the reaction was stirred at 25° C. for 12 hrs. LCMS showed the starting material was consumed and a new peak with desired product Ms was detected. The reaction was diluted with water (50 mL) and extracted with DCM (2×30 mL). The combined organic layer were washed with brine (30 mL), dried over Na₂SO₄ and concentrated to give crude product, which was purified by silica gel chromatography (eluting with 0 to 10% ethyl acetate in petroleum ether) to give Compound 9 (0.6 g, yield 86.58%) as a white solid.

¹H NMR (400 MHz, CHLOROFORM-d) δ-0.05 (s, 9H), 0.60-0.77 (m, 2H), 3.07-3.29 (m, 2H), 4.57-4.83 (m, 4H), 6.76 (d, J=9.03 Hz, 2H), 7.22 (d, J=9.03 Hz, 2H), 7.50 (s, 2H), 7.77 (d, J=15.56 Hz, 2H)

¹⁹F NMR (400 MHz, CHLOROFORM-d) δ −62.5

### General Procedure for Preparation of Compound 3A-ET42365-568

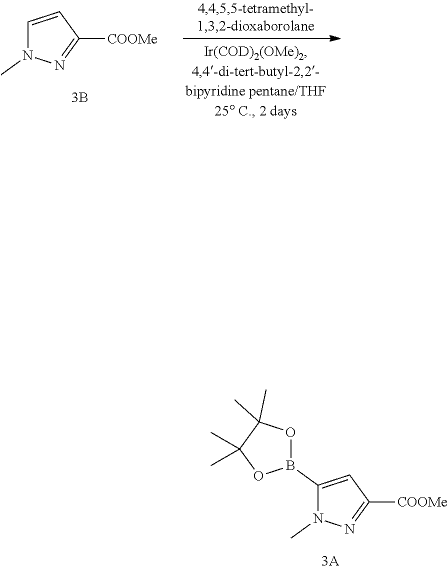

To a solution of (1,5-cyclooctadiene)(methoxy)iridium(I) dimer (212.85 mg, 321.11 μmol, 0.03 eq) and 4,4,5,5-tetramethyl-1,3,2-dioxaborolane (2.05 g, 16.06 mmol, 2.33 mL, 1.5 eq) in pentane (15 mL) was added 4,4′-di-tertbutyl-2,2′-bipyridine (215.46 mg, 802.77 μmol, 0.075 eq) under nitrogen and the mixture was stirred at 25° C. for 20 minutes. To this mixture was added a solution of Compound 3B (1.5 g, 10.70 mmol, 1 eq) in pentane (9 mL) and THF (6 mL), the mixture was stirred at 25° C. for 48 hrs. LCMS showed the starting material was consumed and a new peak with desired product Ms was detected. The reaction was concentrated to give Compound 3A (3 g, crude) as orange solid, which was used to the next step directly without further purification.

$$LCMS ( ESI + ) : RT : 0.169 min , m / z 267.2 {( M + H )}^{+} .$$

5-95AB_2_min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Compound 10-ET42365-576

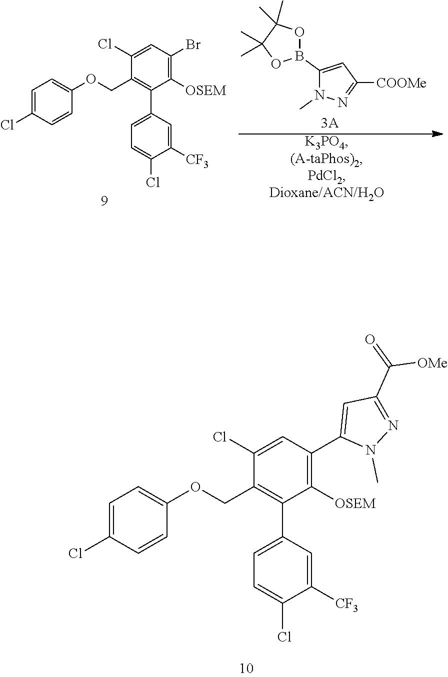

To a solution of Compound 9 (0.6 g, 913.50 μmol, 1 eq), Compound 3A (729.24 mg, 2.74 mmol, 3 eq) and K₃PO₄ (387.82 mg, 1.83 mmol, 2 eq) in a mixture solution of dioxane (12 mL), CH₃CN (12 mL) and H₂O (6 mL) was added 4-ditert-butylphosphanyl-N,Ndimethyl-aniline;dichloropalladium (64.68 mg, 91.35 μmol, 0.1 eq) under nitrogen, the reaction was stirred at 82° C. for 12 hrs. LCMS showed the starting material was consumed and a new peak with desired product Ms was detected. The reaction was filtered and the filtrate was concentrated to give the crude product. The crude product was purified by column chromatography on silica gel (eluted with petroleum ether/ethyl acetate=10/1 to 3/1) to give Compound 10 (330 mg, yield 50.45%) as a yellow solid.

¹H NMR (400 MHz, CHLOROFORM-d) δ −0.11 (s, 9H), 0.48-0.62 (m, 2H), 2.81-2.92 (m, 2H), 3.95 (d, J=6.00 Hz, 6H), 4.37 (s, 2H), 4.69-4.88 (m, 2H), 6.77-6.83 (m, 2H), 6.94 (s, 1H), 7.22-7.26 (m, 2H), 7.49-7.55 (m, 3H), 7.78 (s, 1H)

### General Procedure for Preparation of Compound 11-ET42365-581

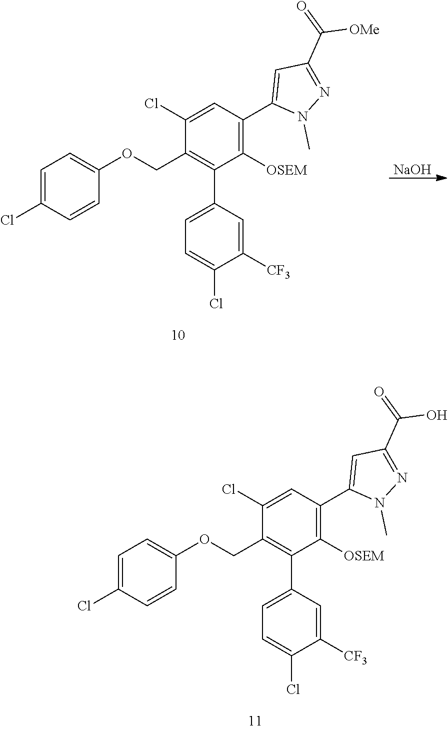

To a solution of Compound 10 (330 mg, 460.86 μmol, 1 eq) in a mixture solution of THF (6 mL), MeOH (2 mL) and H₂O (2 mL) was added NaOH (27.65 mg, 691.30 μmol, 1.5 eq) at 0° C., the reaction was stirred at 20° C. for 12 hrs. LCMS showed the starting material was consumed and a new peak with desired product Ms was detected. The reaction was concentrated to give a residue, diluted with water (30 mL), adjusted to pH=5 with 1 N HCl and extracted with ethyl acetate (3×20 mL). The combined organic layers were washed with brine (30 mL) and dried over Na₂SO₄ and concentrated to give Compound 11 (0.3 g, yield 88.09%) as a yellow solid, which was used to the next step directly without further purification.

¹H NMR (400 MHz, CHLOROFORM-d) δ-0.10 (s, 9H), 0.50-0.62 (m, 2H), 2.83-2.94 (m, 2H), 3.96 (s, 3H), 4.39 (s, 2H), 4.71-4.77 (m, 1H), 4.79-4.86 (m, 1H), 6.75-6.85 (m, 2H), 7.00 (s, 1H), 7.24 (d, J=9.01 Hz, 2H), 7.53 (d, J=9.13 Hz, 3H), 7.78 (s, 1H)

### General Procedure for Preparation of Compound 12-ET42365-585

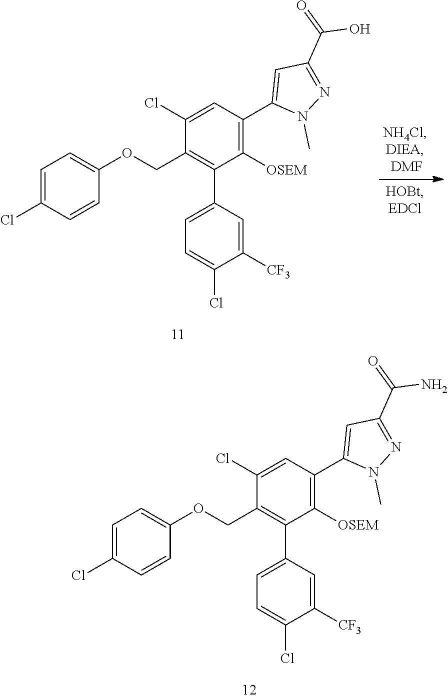

To a solution of Compound 11 (250 mg, 356.12 μmol, 1 eq) and NH₄Cl (95.25 mg, 1.78 mmol, 5 eq) in DMF (5 mL) were added DIPEA (138.07 mg, 1.07 mmol, 186.08 μL, 3 eq) and HOBt (72.18 mg, 534.17 μmol, 1.5 eq), the reaction was stirred at 25° C. for 1 hour, then EDCI (102.40 mg, 534.17 μmol, 1.5 eq) was added and the reaction was stirred at 25° C. for 12 hrs. LCMS showed the starting material was consumed and a new peak with desired product Ms was detected. The reaction was diluted with water (15 mL) and extracted with ethyl acetate (3×10 mL). The combined organic layer were washed with brine (15 mL) and dried over Na₂SO₄ and concentrated to give Compound 12 (200 mg, yield 80.11%) as yellow solid. The product was used to the next step directly without further purification.

¹H NMR (400 MHz, CHLOROFORM-d) δ −0.10 (s, 9H), 0.51-0.61 (m, 2H), 2.84-2.92 (m, 2H), 3.88 (s, 3H), 4.38 (s, 2H), 4.70-4.77 (m, 1H), 4.78-4.85 (m, 1H), 5.41 (br s, 1H), 6.72-6.83 (m, 3H), 6.94 (s, 1H), 7.21-7.26 (m, 2H), 7.48-7.58 (m, 3H), 7.78 (s, 1H)

¹⁹F NMR (400 MHz, CHLOROFORM-d) δ −0.62.5

### General Procedure for Preparation of Compound 8-ET42365-589

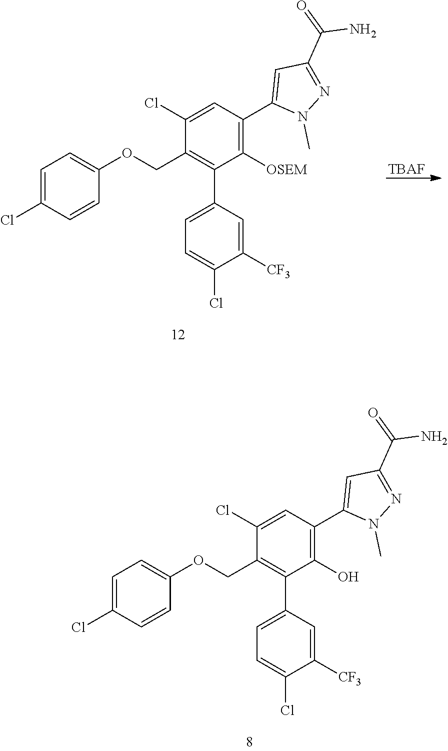

To a solution of Compound 12 (0.2 g, 285.29 μmol, 1 eq) in THF (2 mL) was added TBAF (1 M, 7.13 mL, 25 eq), the reaction was stirred at 50° C. for 12 hrs. LCMS showed the starting material was consumed and a new peak with desired product Ms was detected. The reaction was concentrated to give a residue, diluted with water (15 mL) and extracted with ethyl acetate (2×10 mL). The combined organic layer were washed with brine (15 mL) and dried over Na₂SO₄ and concentrated to give Compound 8 (140 mg, yield 77.38%) as a yellow solid. The product was used to the next step directly without further purification.

$$LCMS ( ESI + ) : RT = 0.727 min , m / z 572.1 {( M + H , M + 2 + H )}^{+} .$$

50-100CD_2_min: LC/MS (The column used for chromatography was Xbridge C18 2.1*50 mm, 5 um. Detection methods are diode array (DAD). MS mode was positive electrospray ionization. MS range was 100-1000. Mobile phase A was 10 mM ammonium bicarbonate in water, and mobile phase B was HPLC grade acetonitrile. The gradient was 50-100% B in 1.50 min 0.50% B in 0.01 min, 50-100% B (0.01-0.80 min) with a hold at 100% B for 0.40 min, 100-50% B (1.20-1.21 min) with a hold at 50% B for 0.29 min. The flow rate was 1.5 mL/min.

### General Procedure for Preparation of P2-2112-4-ET43259-591

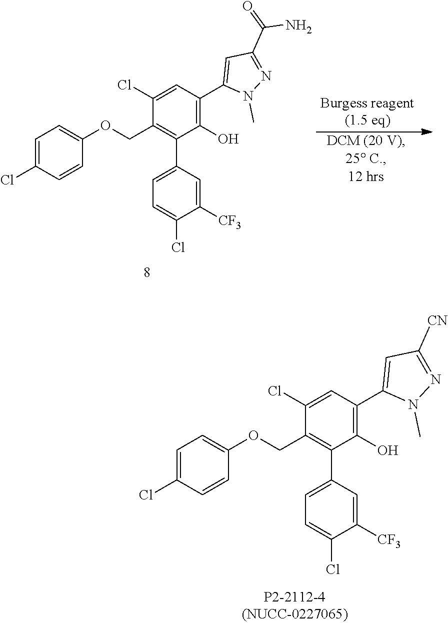 [SMILES: Cn1nc(C(N)=O)cc1-c1cc(Cl)c(COc2ccc(Cl)cc2)c(-c2ccc(Cl)c(C(F)(F)F)c2)c1O ; Cn1nc(C#N)cc1-c1cc(Cl)c(COc2ccc(Cl)cc2)c(-c2ccc(Cl)c(C(F)(F)F)c2)c1O]

To a solution of Compound 8 (140 mg, 245.28 μmol, 1 eq) in DCM (6 mL) was added burgess reagent (116.91 mg, 490.56 μmol, 2 eq) at 0° C., the reaction was stirred at 25° C. for 2 hrs. LCMS showed the starting material was consumed and a new peak with desired product Ms was detected. The reaction was diluted with water (20 mL) and extracted with DCM (2×10 mL). The combined organic layers were washed with brine (15 mL) and dried over Na₂SO₄ and concentrated to give crude product, which was purified by Prep-HPLC (column: Waters Xbridge Prep OBD C18 150*40 mm*10 um; mobile phase: [water (NH₄HCO₃)-ACN]; B %: 65%-95%, 8 min) to give P2-2112-4 (24.4 mg, yield 16.86%) as a grey solid.

¹H NMR (400 MHz, CHLOROFORM-d) δ 3.90 (s, 3H), 4.70-4.77 (m, 1H), 4.79-4.86 (m, 1H), 5.00 (br s, 1H), 6.72-6.81 (m, 3H), 7.20-7.25 (m, 2H), 7.44 (s, 1H), 7.49-7.55 (m, 1H), 7.64 (d, J=8.16 Hz, 1H), 7.75 (d, J=1.38 Hz, 1H)

¹⁹F NMR (400 MHz, CHLOROFORM-d) δ −62.5

$$LCMS ( ESI + ) : RT = 2.516 min , m / z 552. , 554. {( M + H , M + 2 + H )}^{+} .$$

50_100AB_6min-220-254-ELSD: LC/MS (The gradient was 50% B in 0.40 min and 50-100% B at 0.40-3.00 min. hold on 100% B for 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1000.

### General Procedure for Preparation of P2-2112-4_8-ET42365-592

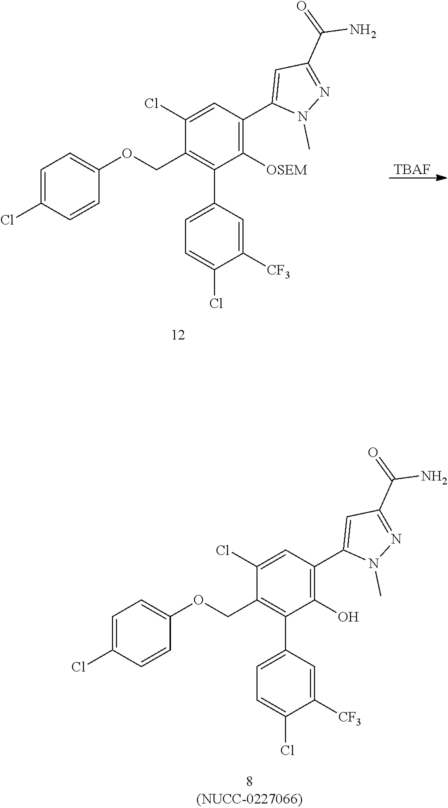

To a solution of Compound 12 (0.1 g, 142.65 μmol, 1 eq) in THF (0.3 mL) was added TBAF (I M, 2.85 mL, 20 eq), the reaction was stirred at 50° C. for 12 hrs. LCMS showed the starting material was consumed and a new peak with desired product Ms was detected. The reaction was concentrated to give a residue, diluted with water (20 mL) and extracted with ethyl acetate (2×10 mL). The combined organic layer were washed with brine (15 mL) and dried over Na₂SO₄ and concentrated to give crude product, which was purified by Prep-HPLC (column: Waters Xbridge Prep OBD C18 150*40 mm*10 um; mobile phase: [water (NH₄HCO₃)-ACN]; B %: 50%-80%, 8 min) to give P2-2112-4_8 (23.5 mg, yield 28.86%) as yellow solid.

¹H NMR (ET42365-592-P1A, 400 MHz, CHLOROFORM-d) δ 3.86 (s, 3H), 4.71-4.77 (m, 1H), 4.79-4.86 (m, 1H), 5.36 (br s, 1H), 6.14 (br s, 1H), 6.74 (br s, 1H), 6.76-6.81 (m, 2H), 6.84 (s, 1H), 7.20-7.25 (m, 2H), 7.39 (s, 1H), 7.50-7.55 (m, 1H), 7.55-7.60 (m, 1H), 7.76 (d, J=1.51 Hz, 1H)

¹⁹F NMR (ET42365-592-P1A, 400 MHz, CHLOROFORM-d) δ −62.7

$$LCMS ( ESI + ) : RT = 1.844 min , m / z 569.8 , 571.8 {( M + K = H )}^{+} .$$

50_100AB_6min-220-254-ELSD: LC/MS (The gradient was 50% B in 0.40 min and 50-100% B at 0.40-3.00 min, hold on 100% B for 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1000.

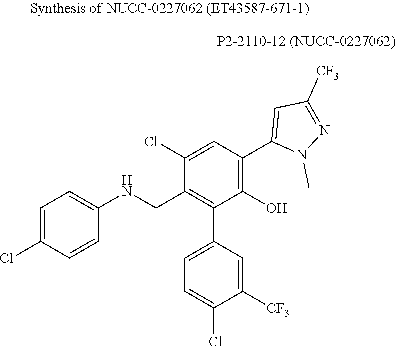 [SMILES: Cn1nc(C(F)(F)F)cc1-c1cc(Cl)c(CNc2ccc(Cl)cc2)c(-c2ccc(Cl)c(C(F)(F)F)c2)c1O]

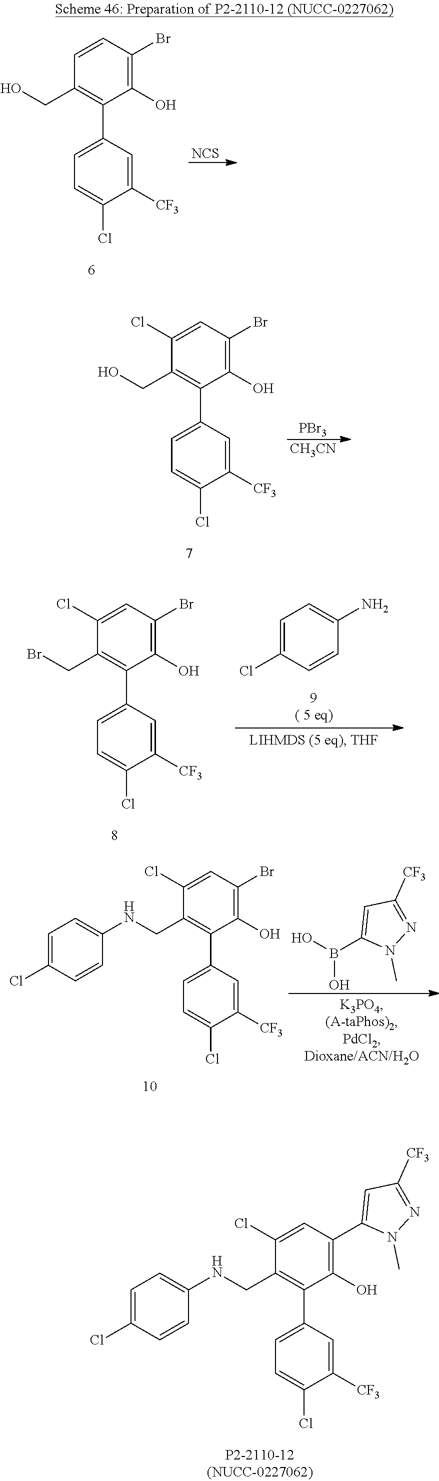

### LCMS Methods:

### Method 1: NEG5-95CD_2_min

LC/MS (The column used for chromatography was Xbridge C18 2.1*50 mm, Sum. Detection methods are diode array (DAD). MS mode was negative electrospray ionization. MS range was 100-1000. Mobile phase A was 10 mm. Ammonium bicarbonate in water, and mobile phase B was HPLC grade acetonitrile. The gradient was 5-95% B in 1.50 min. 5% B in 0.01 min. 5-95% B (0.01-0.70 min), 95% B (0.70-1.16 min), 95-5% B (1.16-1.50 min) with a hold at 5% B for 0.34 min. The flow rate was 1.5 mL/min.

### Method 2: 5_95AB_6min-220-254-ELSD

LC/MS (The gradient was 5% B in 0.40 min and 5-95% B in 2.60 min, hold on 95% B in 1.00 min, and then 95-5% B in 0.01 min, the flow rate was 1.0 ml/min. Mobile phase A was 0.04% trifluoroacetic Acid in water, mobile phase B was 0.02% trifluoroacetic Acid in acetonitrile. The column used for chromatography was a Kinetex C18 2.1*50 mm, 5 um. Detection methods are diode array (DAD), and evaporative light scattering detection (ELSD). MS mode was positive electrospray ionization. MS range was 100-1000.

### Experimentals for Largest Scale Run

### General Procedure for Preparation of Compound 7-ET43587-663

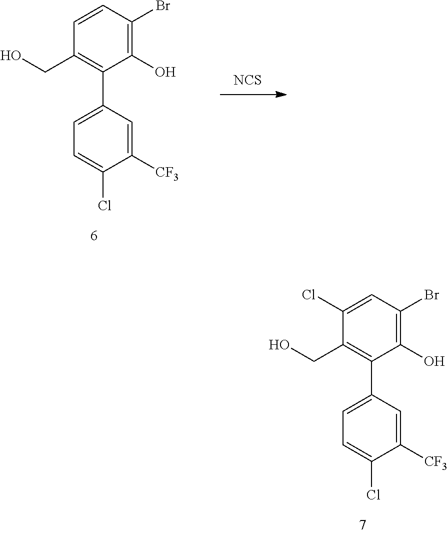 [SMILES: OCc1ccc(Br)c(O)c1-c1ccc(Cl)c(C(F)(F)F)c1 ; "NCS" ; OCc1c(Cl)cc(Br)c(O)c1-c1ccc(Cl)c(C(F)(F)F)c1]

To a solution of Compound 6 (2 g, 5.24 mmol, 1 eq) in AcOH (40 mL) was added NCS (769.90 mg, 5.77 mmol, 1.1 eq) in portions at 0° C. the reaction mixture was stirred at 25° C. for 12 hrs. LCMS showed all the starting material was consumed and a new peak with desired product MS was detected. The reaction mixture was concentrated under reduced pressure to remove AcOH, diluted with water (40 mL) and extracted with ethyl acetate (3×30 mL). The organic layer was separated and the combined organic layer was washed with brine (2×30 mL), dried over Na₂SO₄, filtered and concentrated under reduced pressure to give Compound 7 (1.5 g, yield 61.91%) as a white solid, which was used to the next step without further purification.

$$LCMS ( ESI + ) : RT = 0.89 min , m / z 414.9 {( M - H )}^{-} .$$

### NEG5-95CD_2min

LC/MS (The column used for chromatography was Xbridge C18 2.1*50 mm, 5 um. Detection methods are diode array (DAD). MS mode was negative electrospray ionization. MS range was 100-1000. Mobile phase A was 10 mm. Ammonium bicarbonate in water, and mobile phase B was HPLC grade acetonitrile. The gradient was 5-95% B in 1.50 min. 5% B in 0.01 min, S-95% B (0.01-0.70 min), 95% B (0.70-1.16 min), 95-5% B (1.16-1.50 min) with a bold at 5% B for 0.34 min. The flow rate was 1.5 mL/min.

### General Procedure for Preparation of Compound 8-ET43587-666

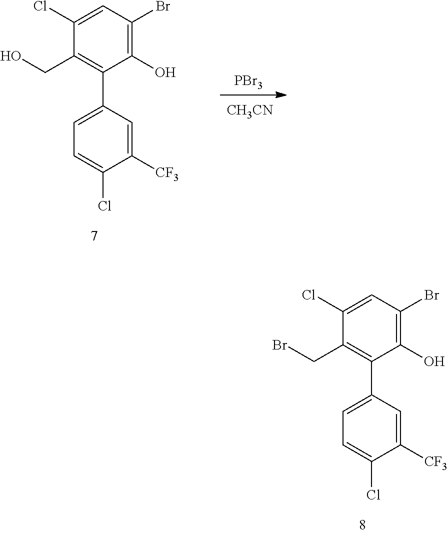 [SMILES: OCc1c(Cl)cc(Br)c(O)c1-c1ccc(Cl)c(C(F)(F)F)c1 ; "PBr3" ; "CH3CN" ; Oc1c(Br)cc(Cl)c(CBr)c1-c1ccc(Cl)c(C(F)(F)F)c1]

To a solution of Compound 7 (200 mg, 480.75 μmol, 1 eq) in ACN (4 mL) was added PBr₃ (78.08 mg, 288.45 μmol, 0.6 eq) at 0° C. the reaction mixture was stirred at 25° C. for 12 hrs. LCMS showed all the starting material was consumed and a new peak with desired product MS was detected. The reaction mixture was diluted with water (20 mL) and extracted with ethyl acetate (3×20 ml). The organic layer was separated and the combined organic layer was washed with brine (2×20 mL), dried over Na₂SO₄, filtered and concentrated under reduced pressure to give Compound 8 (200 mg, yield 78.18%) as a colorless oil, which was used to the next step without further purification.

$$LCMS ( ESI + ) : RT = 0.993 min , m / z 476.8 {( M - H )}^{-} .$$

### NEG5-95CD_2_min

LC/MS (The column used for chromatography was Xbridge C18 2.1*50 mm, 5 um. Detection methods are diode array (DAD). MS mode was negative electrospray ionization. MS range was 100-1000. Mobile phase A was 10 mm. Ammonium bicarbonate in water, and mobile phase B was HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min) with a hold at 5% B for 0.34 min. The flow rate was 1.5 mL/min.

### General Procedure for Preparation of Compound 10-ET43587-667

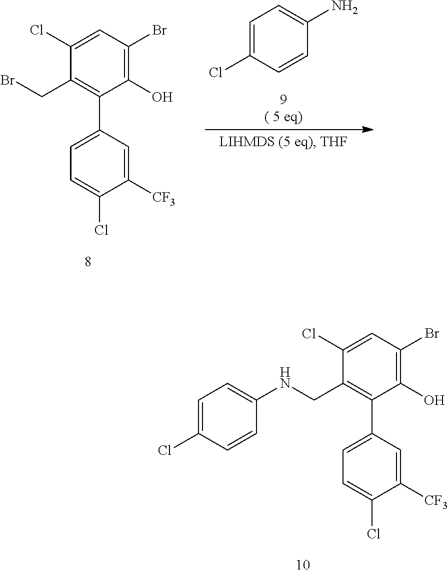 [SMILES: Oc1c(Br)cc(Cl)c(CBr)c1-c1ccc(Cl)c(C(F)(F)F)c1 ; Nc1ccc(Cl)cc1 ; Oc1c(Br)cc(Cl)c(CNc2ccc(Cl)cc2)c1-c1ccc(Cl)c(C(F)(F)F)c1]

To a solution of Compound 9 (266.38 mg, 2.09 mmol, 5 eq) in THF (2 mL) was added LiHMDS (1 M, 2.09 mL, 5 eq) dropwise at −70° C., the mixture was stirred at −40° C. for 30 min. Then Compound 10 (200 mg, 417.61 μmol, 1 eq) in THF (2 mL) was added dropwise, the reaction mixture was stirred at 25° C. for 2 hrs. LCMS showed all the starting material was consumed and a new peak with desired product MS was detected. The reaction mixture was quenched with saturated solution of ammonium chloride (20 mL). Organic phase was separated and the aqueous layer was extracted with ethyl acetate (3×20 mL). The organic layer was separated and the combined organic layer was washed with brine (2×20 mL), dried over Na₂SO₄, filtered and concentrated under reduced pressure to give a residue, which was purified by prep-TLC (petroleum ether/ethyl acetate=5/1) to give Compound 10 (130 mg, yield 53.31%) as a black oil.

¹H NMR (ET43587-667-P1A, 400 MHz, CHLOROFORM-d) δ 4.05 (s, 2H), 5.53 (br s, 1H), 6.32-6.37 (m, 2H), 7.07 (d, J=8.88 Hz, 2H), 7.44 (dd, J=8.25, 1.88 Hz, 1H), 7.56 (d, J=8.25 Hz, 1H), 7.62-7.68 (m, 2H)

### General Procedure for Preparation of P2-2110-12 (NUCC-0227062)-ET43587-671

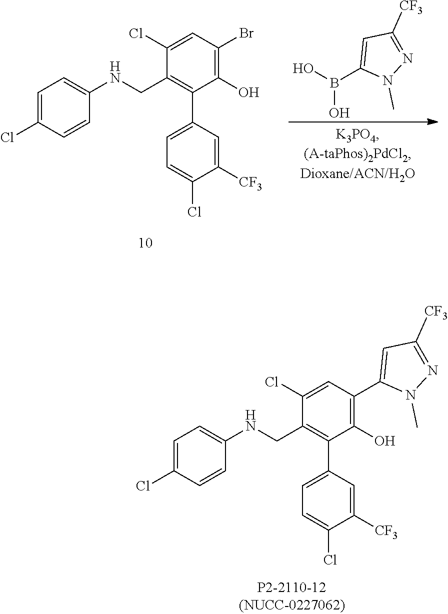 [SMILES: Oc1c(Br)cc(Cl)c(CNc2ccc(Cl)cc2)c1-c1ccc(Cl)c(C(F)(F)F)c1 ; Cn1nc(C(F)(F)F)cc1B(O)O ; Cn1nc(C(F)(F)F)cc1-c1cc(Cl)c(CNc2ccc(Cl)cc2)c(-c2ccc(Cl)c(C(F)(F)F)c2)c1O]

To a solution of Compound 10 (130 mg, 247.35 μmol, 1 eq), [2-methyl-5-(trifluoromethyl) pyrazol-3-yl]boronic acid (124.71 mg, 643.11 μmol, 2.6 €q) and K₃PO₄ (105.01 mg, 494.70 μmol, 2 eq) in a mixture of dioxane (2.6 mL), ACN (2.6 mL) and H₂O (1.3 mL) was added 4-ditert-butylphosphanyl-N,N-dimethyl-aniline; dichloropalladium (17.51 mg, 24.73 μmol, 17.51 μL, 0.1 eq) under nitrogen. The mixture was stirred at 82° C. for 12 hrs. LCMS showed about 12% starting material was remained and 53% desired product was detected. The reaction mixture was diluted with water (20 mL) and extracted with ethyl acetate (3×20 mL). The organic layer was separated and the combined organic layer was washed with brine (2×20 ml), dried over Na₂SO₄, filtered and concentrated under reduced pressure to give a residue, which was purified by Prep-HPLC (column: Waters Xbridge Prep OBD C18 150*40 mm*10 um; mobile phase: [water (NH₄HCO₃)-ACN]; B %: 65%-95%, 8 min) to give P2-2110-12 (43.3 mg, yield 29.43%) as a grey solid.

¹H NMR (ET43587-671-P1A, 400 MHz, DMSO-d6) δ 3.70-3.77 (m, 1H), 3.79 (s, 3H), 3.84-3.91 (m, 1H), 5.99 (t, J=4.20 Hz, 1H), 6.45 (d, J=8.91 Hz, 2H), 6.84 (s, 1H), 7.02 (d, J=8.78 Hz, 2H), 7.55 (s, 1H), 7.65-7.69 (m, 1H), 7.70-7.76 (m, 1H), 7.86 (d, J=1.76 Hz, 1H), 9.18 (s, 1H)

$$LCMS ( ESI + ) : RT = 3.376 min , m / z 595.9 {( M + H )}^{+} .$$

### 5_95AB_6min-220-254-ELSD

LC/MS (The gradient was 5% B in 0.40 min and 5-95% B in 2.60 min, hold on 95% B in 1.00 min, and then 95-5% B in 0.01 min, the flow rate was 1.0 ml/min. Mobile phase A was 0.04% trifluoroacetic Acid in water, mobile phase B was 0.02% trifluoroacetic Acid in acetonitrile. The column used for chromatography was a Kinetex C18 2.1*50 mm, Sum. Detection methods are diode array (DAD), and evaporative light scattering detection (ELSD). MS mode was positive electrospray ionization. MS range was 100-1000.

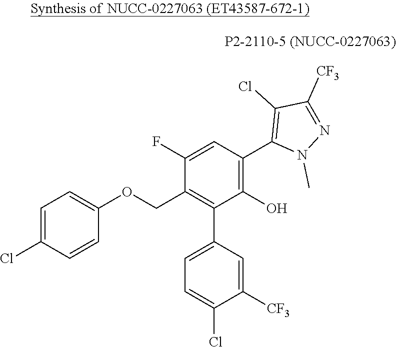 [SMILES: Cn1nc(C(F)(F)F)c(Cl)c1-c1cc(F)c(COc2ccc(Cl)cc2)c(-c2ccc(Cl)c(C(F)(F)F)c2)c1O]

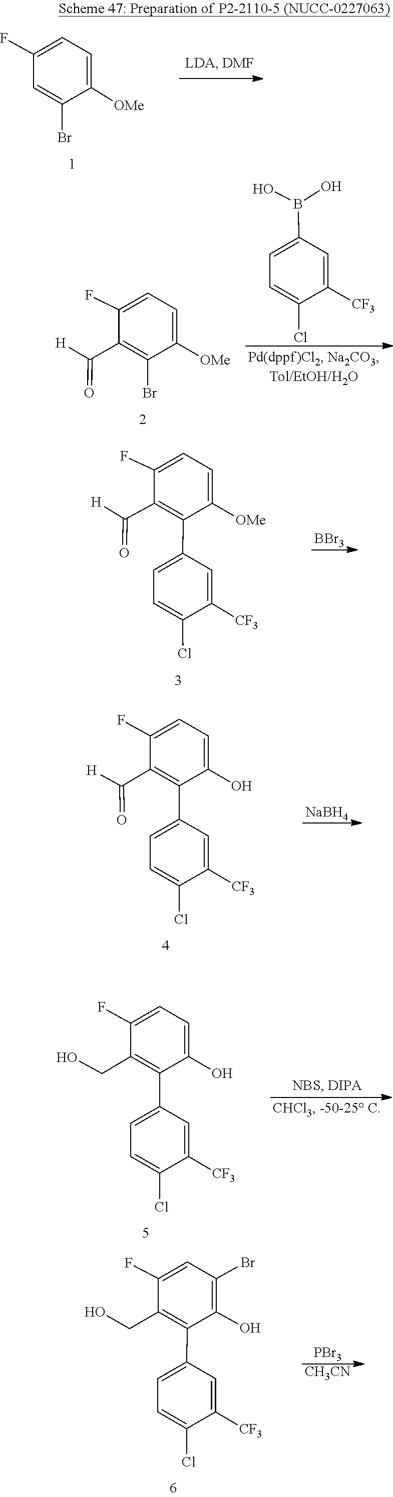

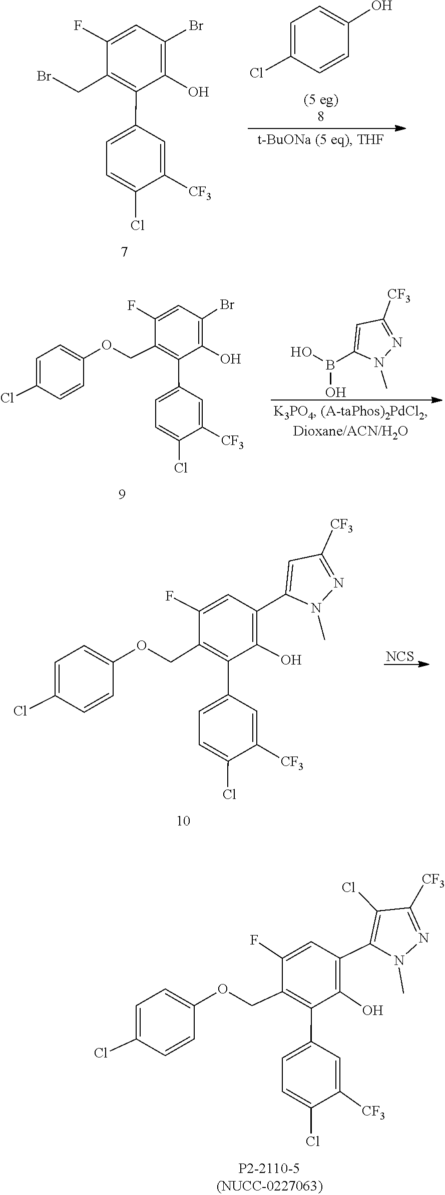

### LCMS Methods:

### Method 1: 5_95AB_6min-220-254-ELSD

LC/MS (The gradient was 5% B in 0.40 min and 5-95% B in 2.60 min, hold on 95% B in 1.00 min, and then 95-5% B in 0.01 min, the flow rate was 1.0 ml/min. Mobile phase A was 0.04% trifluoroacetic Acid in water, mobile phase B was 0.02% trifluoroacetic Acid in acetonitrile. The column used for chromatography was a Kinetex C18 2.1*50 mm, Sum. Detection methods are diode array (DAD), and evaporative light scattering detection (ELSD). MS mode was positive electrospray ionization. MS range was 100-1000.

### Experimentals for Largest Scale Run

### General Procedure for Preparation of Compound 2-ET43587-622

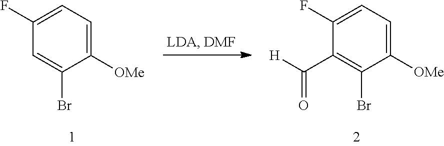 [SMILES: COc1ccc(F)cc1Br ; COc1ccc(F)c(C=O)c1Br]

To a solution of Compound 1 (10 g, 48.77 mmol, 1 eq) in THF (100 mL) was added LDA (2 M, 26.83 mL, 1.1 eq) dropwise at −70° C. After stirring 30 mins at −70° C., DMF (7.13 g, 97.55 mmol, 7.51 mL, 2 eq) was added dropwise. The mixture was stirred at −40° C. for one hour. TLC (petroleum ether/ethyl acetate=5/1) showed all the starting material was consumed and a new spot was generated. The reaction mixture was quenched with saturated solution of ammonium chloride (100 mL). Organic phase was separated and the aqueous layer was extracted with ethyl acetate (3×80 mL). The combined organic layers were washed with brine (2×80 mL), dried over sodium sulfate, filtered and concentrated under reduced pressure to give a residue. Then the residue diluted with MTBE (50 mL) and stirred for 3 hrs. The solid was filtered and dried under high vacuum to obtain Compound 2 (9 g, yield 71.26%) as a white solid.

¹H NMR (ET43587-622-P1A, 400 MHz, CHLOROFORM-d) δ 3.93 (s, 3H), 7.05-7.18 (m, 2H), 10.38 (s, 1H)

### General Procedure for Preparation of Compound 3-ET43587-643

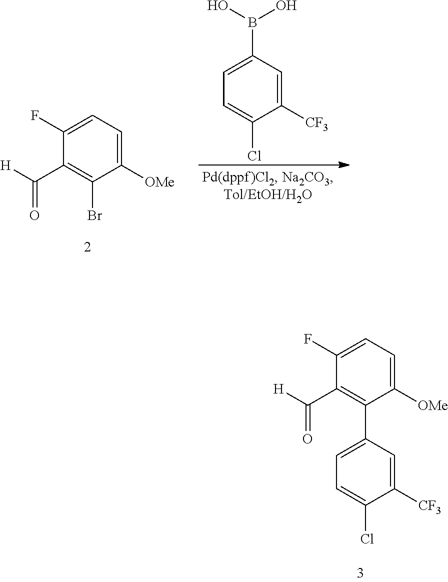 [SMILES: COc1ccc(F)c(C=O)c1Br ; OB(O)c1ccc(Cl)c(C(F)(F)F)c1 ; COc1ccc(F)c(C=O)c1-c1ccc(Cl)c(C(F)(F)F)c1]

To a solution of Compound 2 (9 g, 38.62 mmol, 1 eq), Na₂CO₃ (8.19 g, 77.24 mmol, 2 eq) and [4-chloro-3-(trifluoromethyl)phenyl]boronic acid (10.40 g, 46.35 mmol, 1.2 eq) in a mixture of toluene (300 mL), EtOH (60 mL) and H₂O (15 mL) was added Pd(dppf)Cl₂·CH₂Cl₂ (1.58 g, 1.93 mmol, 0.05 eq) under nitrogen, the reaction mixture was stirred at 85° C. for 12 hrs. LCMS showed all the starting material was consumed and a new peak with desired product MS was detected. The reaction mixture was filtered and concentrated under reduced pressure to remove toluene and EtOH. Then the mixture was diluted with water (100 mL) and extracted with ethyl acetate (3×100 mL). The organic layer was separated and the combined organic layer was washed with brine (2×100 mL), dried over Na₂SO₄, filtered and concentrated under reduced pressure to give a residue, which was purified by chromatography on silica gel (eluted with petroleum ether/ethyl acetate=50/1 to 5/1) to give Compound 3 (11 g. yield 77.05%) as a white solid.

¹H NMR (ET43587-643-P1A, 400 MHz, DMSO-d6) δ 3.72 (s, 3H), 7.44-7.51 (m, 2H), 7.58 (dd, J=8.25, 1.88 Hz, 1H), 7.73 (d, J=1.88 Hz, 1H), 7.77 (d, J=8.25 Hz, 1H), 9.88 (s, 1H)

### General Procedure for Preparation of Compound 4-ET43587-633

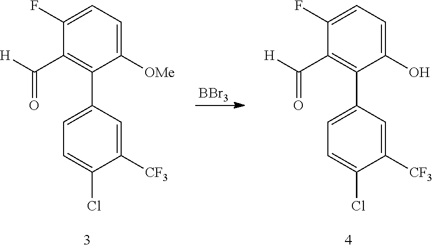

To a solution of Compound 3 (0.9 g, 2.71 mmol, 1 eq) in DCM (9 mL) was added BBr₃ (2.37 g, 9.47 mmol, 912.35 μL, 3.5 eq) dropwise at 0° C., the mixture was stirred at 0° C. for one hour. LCMS showed about 7% of starting material was remaining and a new peak with desired product MS was detected. The mixture was quenched with saturated solution of sodium bicarbonate to pH=7. Organic phase was separated and the aqueous layer was extracted with ethyl acetate (3×20 mL). The combined organic layers were dried over sodium sulfate, filtered and concentrated under reduced pressure to give Compound 4 (650 mg, yield 60.32%) as an off-white solid, which was used to the next step without further purification.

¹H NMR (ET43587-633-P1A, 400 MHz, DMSO-d6) δ 7.20-7.34 (m, 2H), 7.57 (dd, J=8.22, 1.82 Hz, 1H), 7.70-7.79 (m, 2H), 9.89 (s, 1H), 9.96 (s, 1H)

### General Procedure for Preparation of Compound 5-ET43587-642

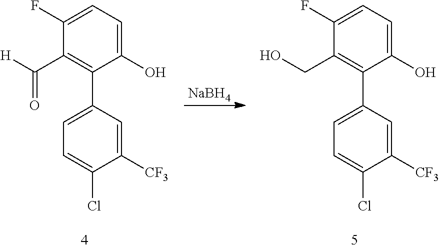 [SMILES: O=Cc1c(F)ccc(O)c1-c1ccc(Cl)c(C(F)(F)F)c1 ; "NaBH4" ; OCc1c(F)ccc(O)c1-c1ccc(Cl)c(C(F)(F)F)c1]

To a solution of Compound 4 (650 mg, 2.04 mmol, 1 eq) in MeOH (13 mL) was added NaBH₄ (61.74 mg, 1.63 mmol, 0.8 eq) at 0° C., the mixture was stirred at 0° C. for one hour. LCMS showed all the starting material was consumed and a new peak with desired product MS was detected. The reaction mixture was quenched with saturated solution of ammonium chloride (20 mL) and concentrated under reduced pressure to remove methanol. The water phase was extracted with ethyl acetate (3×20 mL). The combined organic layers were dried over Na₂SO₄, filtered and concentrated under reduced pressure to give a residue, which was purified by chromatography on silica gel (eluted with petroleum ether/ethyl acetate=50/1 to 2/1) to give Compound 5 (570 mg, yield 78.43%) as a white solid.

¹H NMR (ET43587-642-PIC, 400 MHz, DMSO-d6) δ 4.14 (br d, J=2.50 Hz, 2H), 5.00 (t, J=4.82 Hz, 1H), 6.89 (dd, J=8.88, 4.75 Hz, 1H), 7.08 (t, J=9.13 Hz, 1H), 7.58-7.67 (m, 1H), 7.77 (br d, J=9.63 Hz, 2H), 9.48 (s, 1H)

### General Procedure for Preparation of Compound 6-ET43587-652

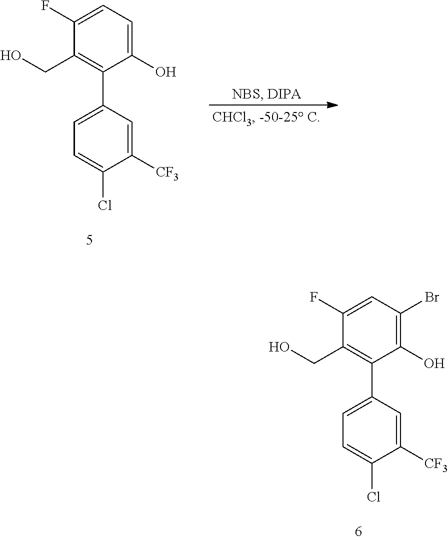

To a solution of Compound 5 (570 mg, 1.78 mmol, 1 eq) and DIPA (539.61 mg, 5.33 mmol, 753.65 μL, 3 eq) in CHCl₃ (12 mL) was added NBS (348.01 mg, 1.96 mmol, 1.1 eq) at −50° C. The mixture was stirred at 25° C. for 12 hrs. LCMS showed all the starting material was consumed and a new peak with desired product MS was detected. The reaction mixture was diluted with water (30 mL) and extracted with ethyl acetate (3×30 mL). The organic layer was separated and the combined organic layer was washed with brine (2×30 mL), dried over Na₂SO₄, filtered and concentrated under reduced pressure to give Compound 6 (500 mg, yield 63.36%) as a colorless oil, which was used to the next step without further purification.

¹H NMR (ET43587-652-P1A, 400 MHz, CHLOROFORM-d) δ 4.41 (br s, 2H), 5.43 (s, 1H), 7.35 (d, J=8.76 Hz, 1H), 7.52 (dd, J=8.19, 1.81 Hz, 1H), 7.61 (d, J=8.25 Hz, 1H), 7.73 (d, J=1.63 Hz, 1H)

### General Procedure for Preparation of Compound 7-ET43587-655

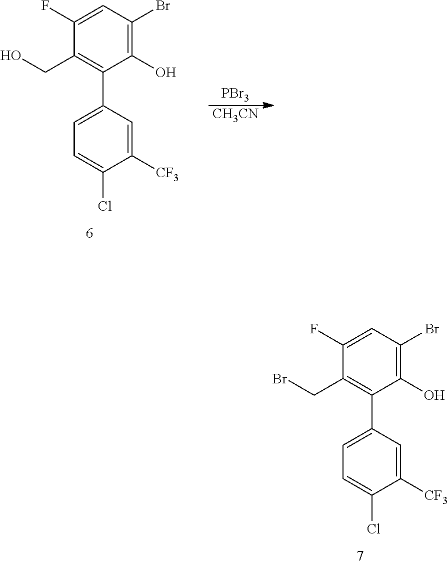

To a solution of Compound 6 (500 mg, 1.25 mmol, 1 eg) in ACN (10 mL) was added PBr₃ (203.24 mg, 750.82 μmol, 0.6 eq) at 0° C., the mixture was stirred at 25° C. for 12 hrs. LCMS showed all the starting material was consumed and a new peak with desired product MS was detected. The reaction mixture was diluted with water (20 mL) and extracted with ethyl acetate (3×20 mL). The organic layer was separated and the combined organic layer was washed with brine (20 mL), dried over Na₂SO₄, filtered and concentrated under reduced pressure to give Compound 7 (570 mg, yield 78.8%) as a colorless oil, which was used to the next step without further purification.

¹H NMR (ET43587-655-P1A, 400 MHz, CHLOROFORM-d) δ 4.14-4.21 (m, 2H), 4.58 (br s, 1H), 7.36 (d, J=8.63 Hz, 1H), 7.55 (dd, J=8.25, 1.88 Hz, 1H), 7.66 (d, J=8.25 Hz, 1H), 7.74 (d, J=2.00 Hz, 1H)

### General Procedure for Preparation of Compound 9-ET43587-664

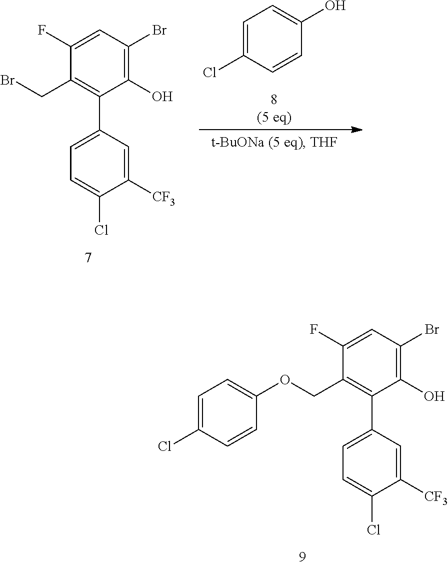 [SMILES: Oc1c(Br)cc(F)c(CBr)c1-c1ccc(Cl)c(C(F)(F)F)c1 ; Oc1ccc(Cl)cc1 ; Oc1c(Br)cc(F)c(COc2ccc(Cl)cc2)c1-c1ccc(Cl)c(C(F)(F)F)c1]

To a solution of Compound 8 (792.25 mg, 6.16 mmol, 5 eq) in a mixture of THF (6 mL) and DMF (6 mL) was added t-BuONa (592.26 mg, 6.16 mmol, 5 eg), the mixture was stirred at 25° C. for 30 min. Then Compound 7 (570 mg, 1.23 mmol, 1 eq) in THF (3 ml) was added dropwise, the reaction was stirred at 25° C. for 12 hrs. LCMS showed all the starting material was consumed and a new peak with desired product MS was detected. The reaction mixture was diluted with water (30 mL) and extracted with ethyl acetate (3×30 mL). The organic layer was separated and the combined organic layer was washed with brine (2×30 mL), dried over Na₂SO₄, filtered and concentrated under reduced pressure to give a residue, which was purified by chromatography on silica gel (eluted with petroleum ether/ethyl acetate=50/1 to 5/1) to give Compound 9 (500 mg, yield 71.57%) as a colorless oil.

¹H NMR (ET43587-664-P1A, 400 MHz, CHLOROFORM-d) δ 4.68 (br s, 2H), 5.40 (s, 1H), 6.70-6.78 (m, 2H), 7.18-7.24 (m, 2H), 7.40 (d, J=8.38 Hz, 1H), 7.44-7.50 (m, 1H), 7.51-7.56 (m, 1H), 7.72 (d, J=1.50 Hz, 1H)

### General Procedure for Preparation of Compound 10-ET43587-675

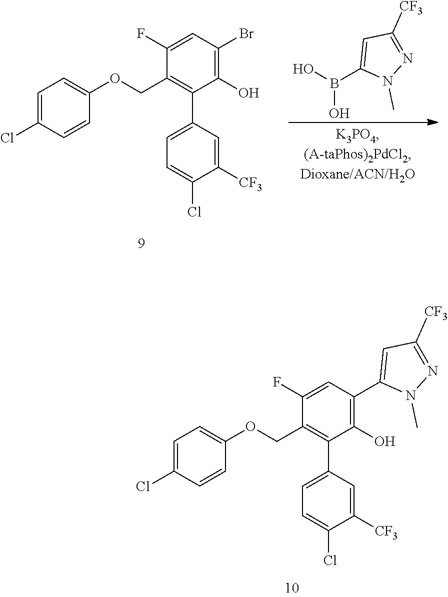 [SMILES: Oc1c(Br)cc(F)c(COc2ccc(Cl)cc2)c1-c1ccc(Cl)c(C(F)(F)F)c1 ; Cn1nc(C(F)(F)F)cc1B(O)O ; Cn1nc(C(F)(F)F)cc1-c1cc(F)c(COc2ccc(Cl)cc2)c(-c2ccc(Cl)c(C(F)(F)F)c2)c1O]

To a solution of Compound 9 (400 mg, 784.15 μmol, 1 eq), K₃PO₄ (332.90 mg, 1.57 mmol, 2 eq) and [2-methyl-5-(trifluoromethyl) pyrazol-3-yl]boronic acid (410.57 mg, 2.12 mmol, 2.7 eq) in a mixture of dioxane (8 mL), CH₃CN (8 mL) and H₂O (4 mL) was added 4-ditertbutylphosphanyl-N,N-dimethyl-aniline;dichloropalladium (55.52 mg, 78.42 μmol, 55.52 μL, 0.1 eq) under nitrogen. The mixture was stirred at 82° C. for 12 hrs. LCMS showed all the starting material was consumed and a new peak with desired product MS was detected. The reaction mixture was diluted with water (20 mL) and extracted with ethyl acetate (3×20 mL). The organic layer was separated and the combined organic layer was washed with brine (2×20 mL), dried over Na₂SO₄, filtered and concentrated under reduced pressure to give a residue, which was purified by chromatography on silica gel (eluted with petroleum ether/ethyl acetate=50/1 to 5/1) to give Compound 10 (350 mg, yield 69.34%) as a pink solid.

¹H NMR (ET43587-675-P1A, 400 MHz, DMSO-d6) δ 3.81 (s, 3H), 4.59-4.87 (m, 2H), 6.82-6.96 (m, 3H), 7.24-7.33 (m, 2H), 7.40 (d, J=9.76 Hz, 1H), 7.65 (dd, J=8.25, 1.88 Hz, 1H), 7.71-7.81 (m, 2H), 9.03 (s, 1H)

### General Procedure for Preparation of P2-2110-5-ET43587-682

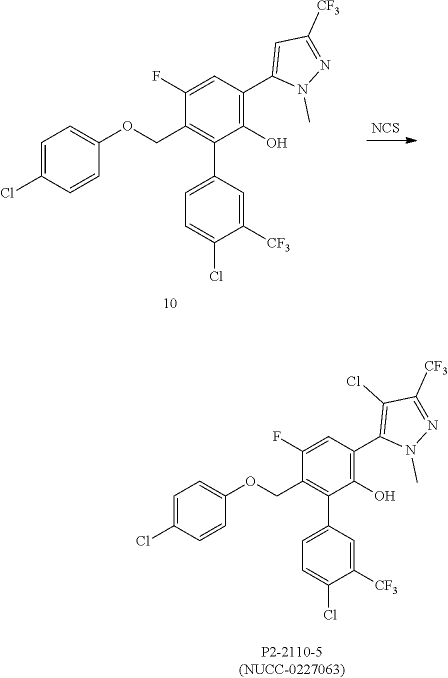 [SMILES: Cn1nc(C(F)(F)F)cc1-c1cc(F)c(COc2ccc(Cl)cc2)c(-c2ccc(Cl)c(C(F)(F)F)c2)c1O ; "NCS" ; Cn1nc(C(F)(F)F)c(Cl)c1-c1cc(F)c(COc2ccc(Cl)cc2)c(-c2ccc(Cl)c(C(F)(F)F)c2)c1O]

To a solution of Compound 10 in CH₃CN (2 mL) was added NCS (34.58 mg, 258.94 μmol, 1.5 eq) at 0° C., the mixture was stirred at 50° C. for 16 hrs. LCMS showed all the starting material was consumed and a new peak with desired product MS was detected. The mixture was purified by Prep-HPLC (column: Waters Xbridge Prep OBD C18 150*40 mm*10 um; mobile phase: [water (NH₄HCO₃)-ACN]; B %: 65%-95%, 8 min) to give P2-2110-5 (35.9 mg, yield 33.38%) as a white solid.

¹H NMR (ET43587-682-P1A, 400 MHz, DMSO-d6) δ 3.80 (s, 3H), 4.58-4.90 (m, 2H), 6.84-6.95 (m, 2H), 7.23-7.33 (m, 2H), 7.46 (d, J=9.76 Hz, 1H), 7.67 (br d, J=7.38 Hz, 1H), 7.76 (br d, J=8.38 Hz, 2H), 9.28 (br s, 1H)

$$LCMS ( ESI + ) : RT = 3.414 min , m / z 615. {( M + H )}^{+} .$$

### 5_95AB_6min-220-254-ELSD

LC/MS (The gradient was 5% B in 0.40 min and 5-95% B in 2.60 min, hold on 95% B in 1.00 min, and then 95-5% B in 0.01 min, the flow rate was 1.0 ml/min. Mobile phase A was 0.04% trifluoroacetic Acid in water, mobile phase B was 0.02% trifluoroacetic Acid in acetonitrile. The column used for chromatography was a Kinetex C18 2.1*50 mm, 5 um. Detection methods are diode array (DAD), and evaporative light scattering detection (ELSD). MS mode was positive electrospray ionization. MS range was 100-1000.

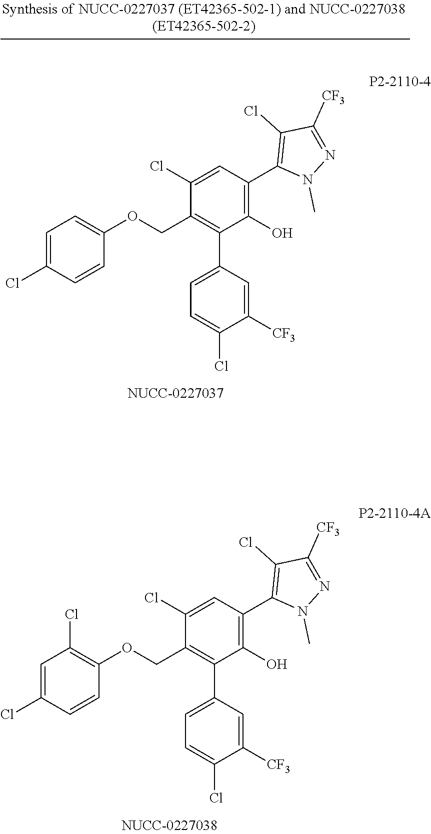 [SMILES: Cn1nc(C(F)(F)F)c(Cl)c1-c1cc(Cl)c(COc2ccc(Cl)cc2)c(-c2ccc(Cl)c(C(F)(F)F)c2)c1O ; Cn1nc(C(F)(F)F)c(Cl)c1-c1cc(Cl)c(COc2ccc(Cl)cc2Cl)c(-c2ccc(Cl)c(C(F)(F)F)c2)c1O]

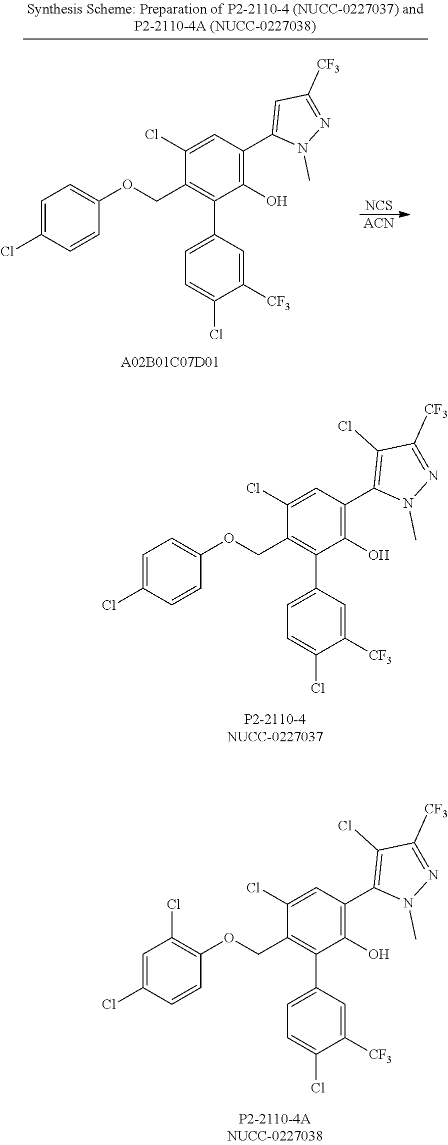 [SMILES: Cn1nc(C(F)(F)F)cc1-c1cc(Cl)c(COc2ccc(Cl)cc2)c(-c2ccc(Cl)c(C(F)(F)F)c2)c1O ; "ACN" ; "NCS" ; Cn1nc(C(F)(F)F)c(Cl)c1-c1cc(Cl)c(COc2ccc(Cl)cc2)c(-c2ccc(Cl)c(C(F)(F)F)c2)c1O ; Cn1nc(C(F)(F)F)c(Cl)c1-c1cc(Cl)c(COc2ccc(Cl)cc2Cl)c(-c2ccc(Cl)c(C(F)(F)F)c2)c1O]

### Chemical Synthesis

50_100AB_6min-220-254-ELSD: LC/MS (The gradient was 50% B in 0.40 min and 50-100% B at 0.40-3.00 min, hold on 100% B for 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1000.

### Experimentals for Largest Scale Run

### General Procedure for Preparation of P2-2110-4 and P2-2110-4A-ET43365-502

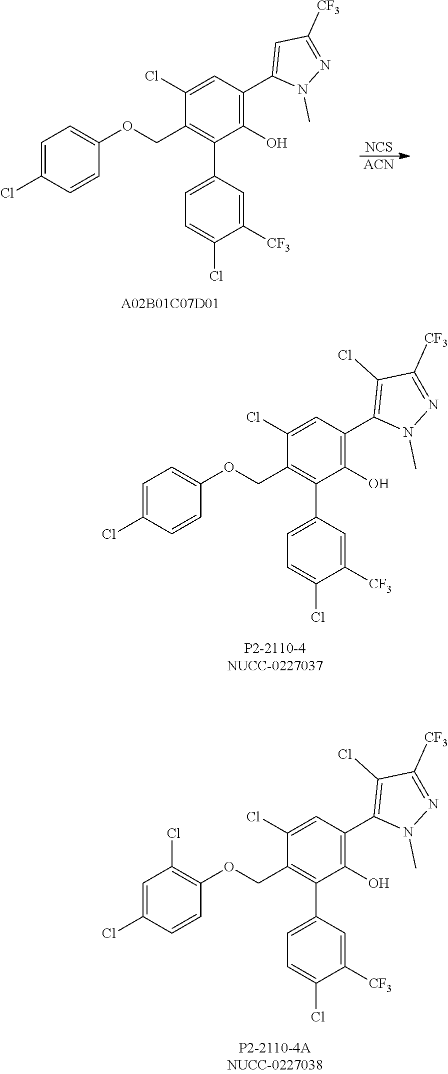 [SMILES: Cn1nc(C(F)(F)F)cc1-c1cc(Cl)c(COc2ccc(Cl)cc2)c(-c2ccc(Cl)c(C(F)(F)F)c2)c1O ; "ACN" ; "NCS" ; Cn1nc(C(F)(F)F)c(Cl)c1-c1cc(Cl)c(COc2ccc(Cl)cc2)c(-c2ccc(Cl)c(C(F)(F)F)c2)c1O ; Cn1nc(C(F)(F)F)c(Cl)c1-c1cc(Cl)c(COc2ccc(Cl)cc2Cl)c(-c2ccc(Cl)c(C(F)(F)F)c2)c1O]

To a solution of A02B01C07D01 (200 mg, 335.71 μmol, 1 eq) in CH₃CN (5 mL) was added NCS (49.31 mg, 369.28 μmol, 1.1 eq) in portions at 0° C., the reaction was stirred at 20° C. for 12 hrs. LCMS showed the starting material was consumed and a new peak with desired product Ms was detected. The reaction was purified by Prep-HPLC (column: Waters Xbridge Prep OBD CIS 150*40 mm*10 um; mobile phase: [water (NH₄HCO₃)-ACN]; B %: 75%-95%, 8 min) to give P2-2110-4 (111.3 mg, yield 50.24%) as a yellow solid and P2-2110-4A (35.1 mg, yield 15.73%) as a white solid.

### P2-2110-4:

¹H NMR (ET42365-502-P1A, 400 MHz, CHLOROFORM-d) δ 3.85 (d, J=2.75 Hz, 3H), 4.65-4.92 (m, 2H), 4.95 (br s, 1H), 6.78 (d, J=8.88 Hz, 2H), 7.21-7.26 (m, 2H), 7.49 (s, 1H), 7.55 (dd, J=8.19, 1.81 Hz, 1H), 7.65 (br d, J=8.25 Hz, 1H), 7.78 (d, J=1.88 Hz, 1H)

$$LCMS ( ESI + ) : RT = 2.746 min , m / z 631. {( M + H )}^{+} .$$

50_100AB_6min-220-254-ELSD: LC/MS (The gradient was 50% B in 0.40 min and 50-100% B at 0.40-3.00 min, hold on 100% B for 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1000.

### P2-2110-4A:

¹H NMR (ET42365-502-P2A, 400 MHz, CHLOROFORM-d) δ 3.87 (br d, J=5.75 Hz, 3H), 4.68-5.07 (m, 3H), 6.81 (br dd, J=8.38, 4.75 Hz, 1H), 7.16 (br d, J=8.38 Hz, 1H), 7.38 (d, J=2.50 Hz, 1H), 7.50 (s, 1H), 7.57-7.69 (m, 2H), 7.77 (br d, J=5.13 Hz, 1H)

$$LCMS ( ESI + ) : RT = 2.82 min , m / z 664.8 {( M + H )}^{+} .$$

50_100AB_6min-220-254-ELSD: LC/MS (The gradient was 50% B in 0.40 min and 50-100% B at 0.40-3.00 min, hold on 100% B for 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1000.

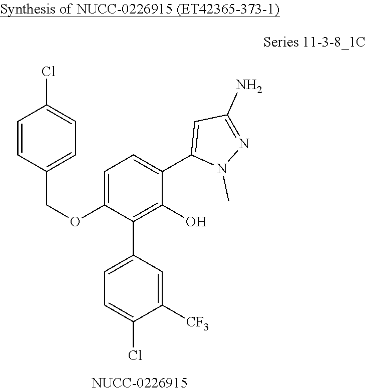 [SMILES: Cn1nc(N)cc1-c1ccc(OCc2ccc(Cl)cc2)c(-c2ccc(Cl)c(C(F)(F)F)c2)c1O]

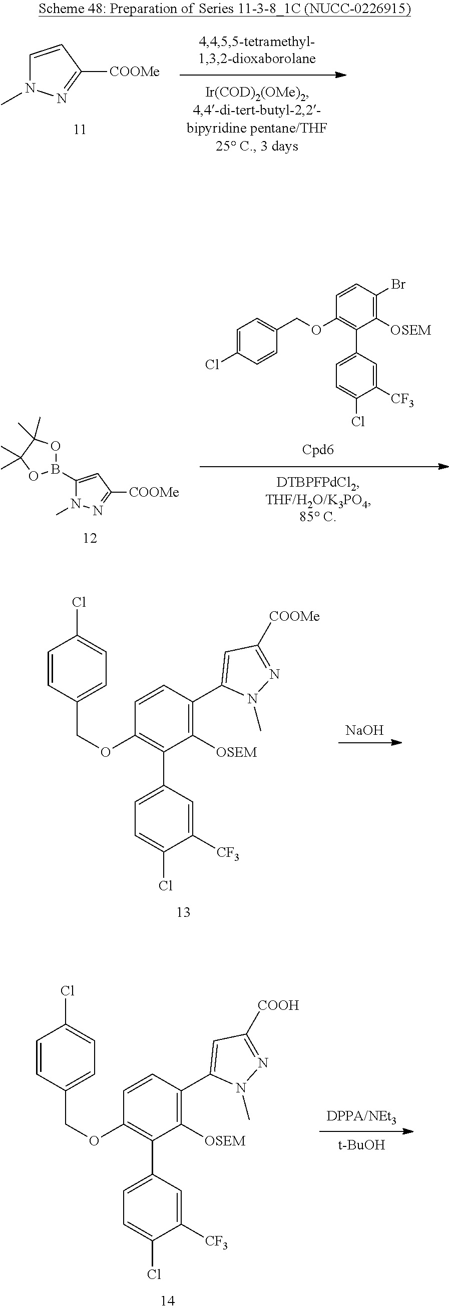

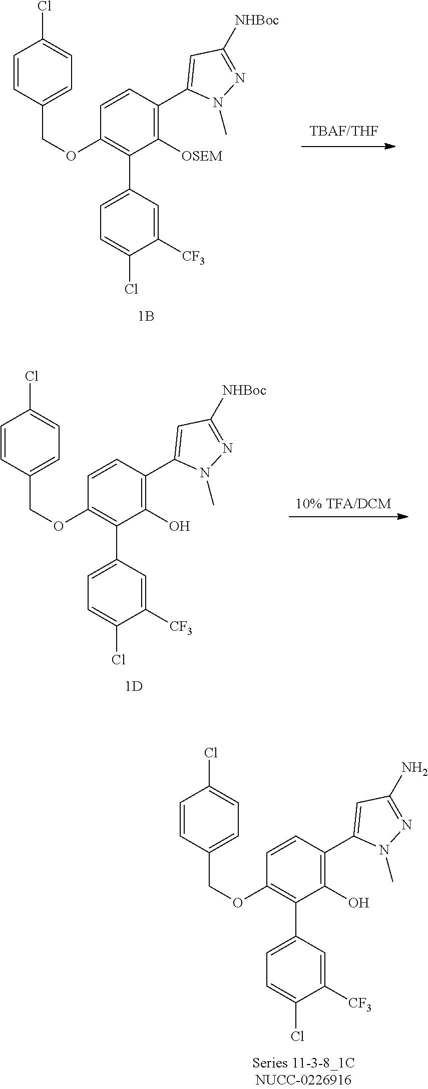

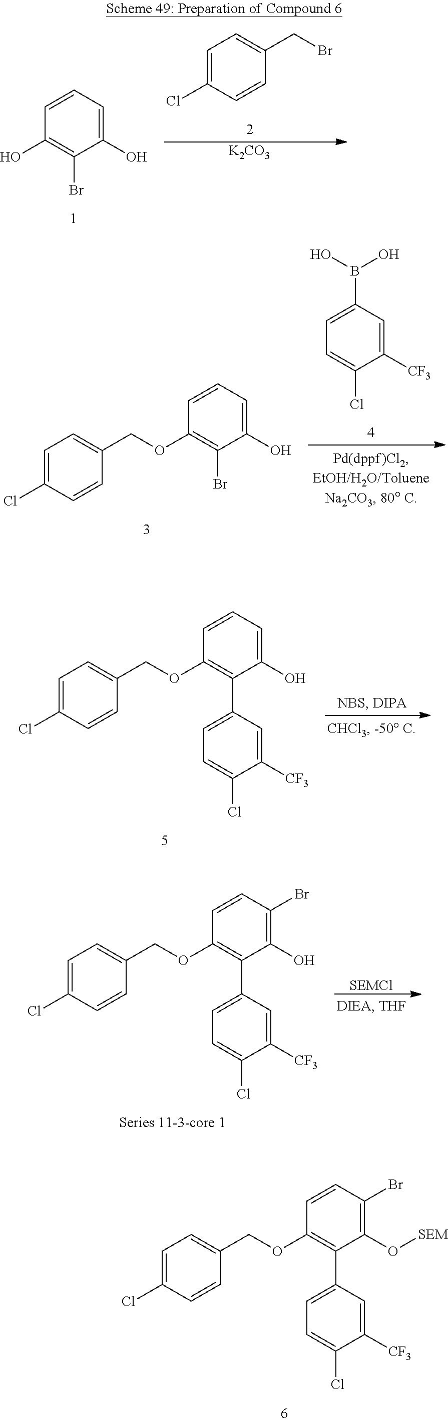

NEG5-95CD 2 min: LC/MS (The column used for chromatography was Xbridge C18 2.1*50 mm, Sum. Detection methods are diode array (DAD). MS mode was negative electrospray ionization. MS range was 100-1000. Mobile phase A was 10 mM Ammonium bicarbonate in water, and mobile phase B was HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min) with a hold at 5% B for 0.34 min. The flow rate was 1.5 mL/min.

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 ml/min.

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

### Experimentals for Largest Scale Run

### General Procedure for Preparation of Compound 3-ET43365-230

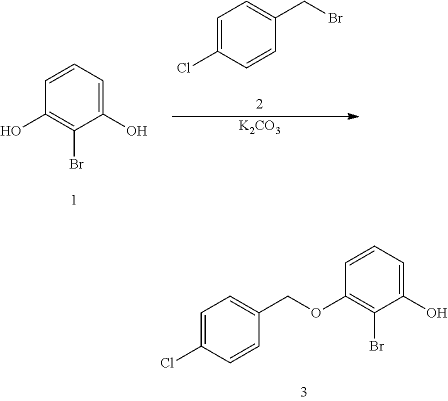 [SMILES: Oc1cccc(O)c1Br ; "K2CO3" ; Clc1ccc(CBr)cc1 ; Oc1cccc(OCc2ccc(Cl)cc2)c1Br]

To a solution of Compound 1 (19.91 g, 105.34 mmol, 3.33 eq) in CH₃CN (200 mL) were added K₂CO₃ (17.49 g, 126.53 mmol, 4 eq) and Compound 2 (6.5 g, 31.63 mmol, 1 eq), the reaction was stirred at 60° C. for 2 hrs. LCMS showed the starting material (Compound 2) was consumed and a new peak with desired product Ms was detected. The reaction was diluted with water (300 mL) and was extracted with ethyl acetate (3×150 mL). The combined organic layers were washed with brine (200 mL), dried over Na₂SO₄ and concentrated to give crude product. The crude product was purified by reversed phase flash to give Compound 3 (6 g, yield 60.49%) as a yellow solid.

### Reversed Phase Flash Method:

Column: 800 g Agela C18
Solvent for sample dissolution about 9 grams of sample dissolved in 50 ml of THF
Flow rate: 120 ml/min
Mobile phase: pure water
Gradient B %: 15-55% 30 min; 55% 25 min
Instrument: Biotage

¹H NMR (ET42365-230-P1A, 400 MHz, CHLOROFORM-d) δ 5.11 (s, 2H), 5.66 (s, 1H), 6.51 (dd, J=8.32, 1.06 Hz, 1H), 6.71 (dd, J=8.25, 1.13 Hz, 1H), 7.15 (t, J=8.25 Hz, 1H), 7.32-7.48 (m, 4H)

### General Procedure for Preparation of Compound 5-ET42365-235

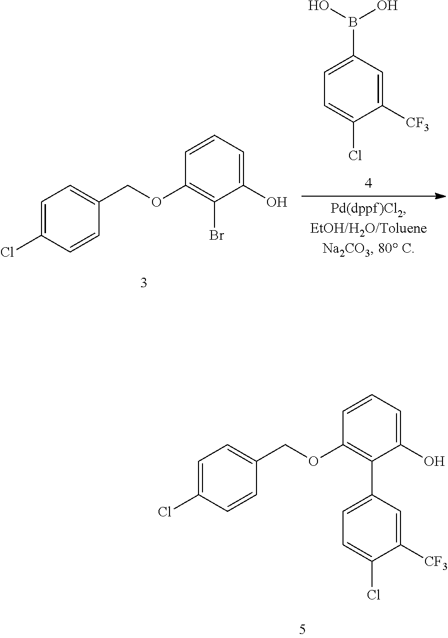 [SMILES: Oc1cccc(OCc2ccc(Cl)cc2)c1Br ; OB(O)c1ccc(Cl)c(C(F)(F)F)c1 ; Oc1cccc(OCc2ccc(Cl)cc2)c1-c1ccc(Cl)c(C(F)(F)F)c1]

To a solution of Compound 3 (6 g, 19.13 mmol, 1 eq), Compound 4 (5.17 g, 24.87 mmol, 1.3 eq) and K₃PO₄ (8.12 g, 38.27 mmol, 2 eq) in a mixture solution of H₂O (24 mL) and THF (120 mL) was added ditert-butyl(cyclopentyl)phosphane;dichloropalladium;iron (872.95 mg, 1.34 mmol, 0.07 eq) under nitrogen, the reaction was stirred at 80° C. for 12 hrs. LCMS showed the starting material was consumed and a new peak with desired Ms was detected. The reaction was filtered and the filtrate was concentrated to give the crude product. The crude product was purified by column chromatography on silica gel (eluted with petroleum ether/ethyl acetate=50/1 to 10/1) to give Compound 5 (5 g, yield 56.91%) as a yellow solid.

¹H NMR (ET42365-235-P1A, 400 MHz, CHLOROFORM-d) δ 4.86 (br s, 1H), 5.00 (s, 2H), 6.64 (t, J=8.82 Hz, 2H), 7.14 (d, J=8.25 Hz, 2H), 7.23 (t, J=8.32 Hz, 1H), 7.29 (d, J=8.38 Hz, 2H), 7.51-7.56 (m, 1H), 7.57-7.62 (m, 1H), 7.79 (d, J=1.38 Hz, 1H)

### General Procedure for Preparation of Series 11-3-core 1-ET42365-317

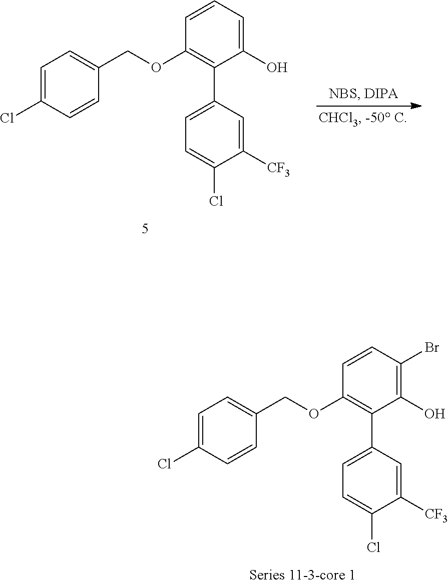 [SMILES: Oc1cccc(OCc2ccc(Cl)cc2)c1-c1ccc(Cl)c(C(F)(F)F)c1 ; Oc1c(Br)ccc(OCc2ccc(Cl)cc2)c1-c1ccc(Cl)c(C(F)(F)F)c1]

To a solution of Compound 5 (2 g, 4.84 mmol, 1 eq) and DIPA (979.53 mg, 9.68 mmol, 1.37 mL, 2 eq) in CHCl₃ (50 mL) was added 1-bromopyrrolidine-2,5-dione (861.46 mg, 4.84 mmol, 1 eq) at −40° C., the reaction was stirred at −40° C. for 4 hrs. LCMS showed the starting material was consumed and a new peak was detected. The reaction was diluted with water (100 mL) and extracted with DCM (3×50 mL). The combined organic layer were washed with brine (100 mL), dried over Na₂SO₄ and concentrated to give crude Series 11-3-core 1 (2 g, yield 71.37%) as a yellow solid. The crude product was used to the next step directly without further purification.

$$LCMS ( ESI + ) : RT = 1.045 min , m / z 490.8 {( M - H )}^{+} .$$

NEG5-95CD_2_min: LC/MS (The column used for chromatography was Xbridge C18 2.1*50 mm, Sum. Detection methods are diode array (DAD). MS mode was negative electrospray ionization. MS range was 100-1000. Mobile phase A was 10 mM Ammonium bicarbonate in water, and mobile phase B was HPLC grade acetonitrile. The gradient was 5-95% B in 1.50 min 0.5% B in 0.01 min. 5-95% B (0.01-0.70 min), 95% B (0.70-1.16 min), 95-5% B (1.16-1.50 min) with a hold at 5% B for 0.34 min. The flow rate was 1.5 ml/min.

### General Procedure for Preparation of Compound 6-ET42365-320

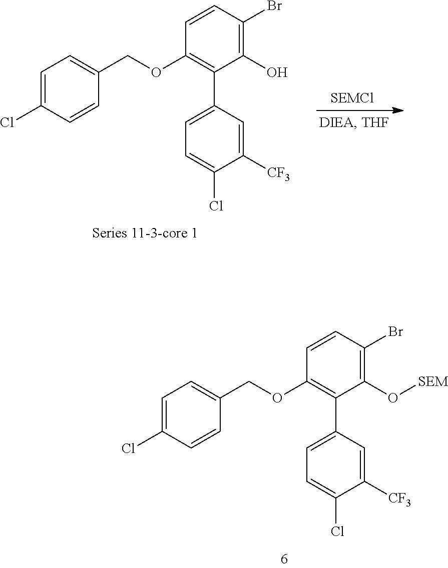 [SMILES: Oc1c(Br)ccc(OCc2ccc(Cl)cc2)c1-c1ccc(Cl)c(C(F)(F)F)c1 ; "SEMCl" ; C[Si](C)(C)CCOCOc1c(Br)ccc(OCc2ccc(Cl)cc2)c1-c1ccc(Cl)c(C(F)(F)F)c1]

To a solution of Series 11-3-core 1 (1 g, 2.03 mmol, 1 eq) and DIEA (787.87 mg, 6.10 mmol, 1.06 mb, 3 eq) in DCM (30 mL) was added SEM-Cl (677.57 mg, 4.06 mmol, 719.29 μL, 2 eq) dropwise at 0° C., the reaction was stirred at 25° C. for 12 hrs. LCMS showed the starting material was consumed and a new peak with desired product Ms was detected. The reaction was diluted with water (60 mL) and was extracted with DCM (3×30 mL). The combined organic layers were washed with brine (30 mL), dried over Na₂SO₄ and concentrated to give crude product. The crude product was purified by silica gel chromatography (eluting with 0 to 15% ethyl acetate in petroleum ether) to give Compound 6 (2 g, yield 71.16%) as a white solid.

$$LCMS ( ESI + ) : RT = 1.289 min , m / z 621. {( M - H )}^{+} .$$

NEG5-95CD 2 min: LC/MS (The column used for chromatography was Xbridge C18 2.1*50 mm, 5 um. Detection methods are diode array (DAD). MS mode was negative electrospray ionization. MS range was 100-1000. Mobile phase A was 10 mM Ammonium bicarbonate in water, and mobile phase B was HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min) with a hold at 5% B for 0.34 min. The flow rate was 1.5 mL/min.

### General Procedure for Preparation of Compound 12-ET42365-324

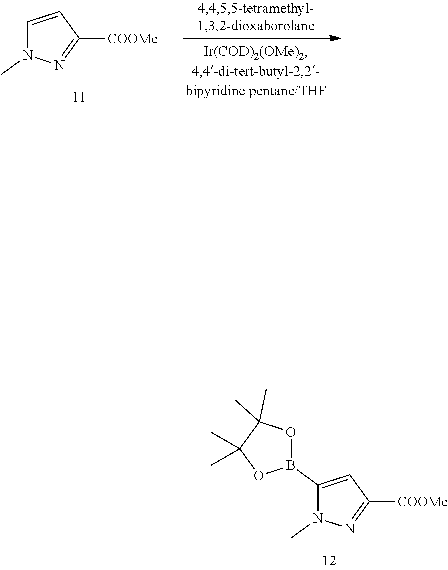

To a solution of (1,5-cyclooctadiene)(methoxy)iridium (I) dimer (354.75 mg, 535.18 μmol, 0.03 eq) and 4,4,5,5-tetramethyl-1,3,2-dioxaborolane (3.42 g, 26.76 mmol, 3.88 mL, 1.5 eq) in pentane (25 mL) was added 4,4′-di-tertbutyl-2,2′-bipyridine (359.10 mg, 1.34 mmol, 0.075 eq) and the mixture was stirred at 25° C. for 20 minutes. To this mixture was added a solution of Compound 11 (2.5 g, 17.84 mmol, 1 eq) in pentane (15 mL) and THF (10 mL) the mixture was stirred at 25° C. for 55 hrs. LCMS showed about 15.3% of the starting material was remaining and a new peak with desired product Ms was detected. The reaction solution was used to the next step directly without work up and purification.

$$LCMS ( ESI + ) : RT = 0.414 min , m / z 267.1 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 ml/min.

### General Procedure for Preparation of Compound 13-ET42365-329

The reactions were conducted in parallel but combined for purification.

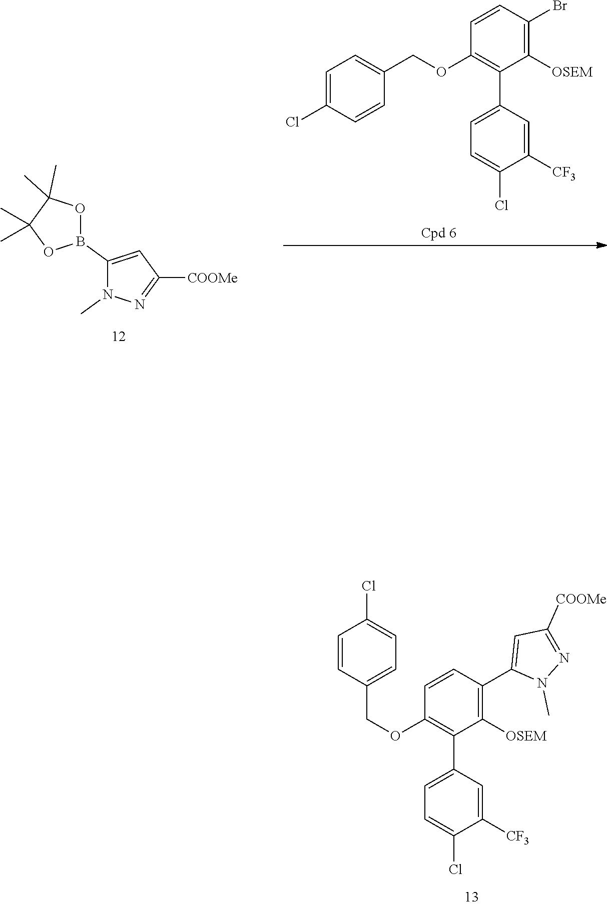

To a solution of Compound 12 (2.57 g, 9.64 mmol, 3 eq), Compound 6 (2 g, 3.21 mmol, 1 eq) and K₂CO₃ (888.28 mg, 6.43 mmol, 2 eq) in a solution of dioxane (50 mL) and H₂O (12.5 mL) was added palladium;tritert-butylphosphane (164.23 mg, 321.35 μmol, 0.1 eq) under nitrogen, the reaction was stirred at 80° C. for 3 hrs. LCMS showed the starting material was consumed and a new peak with desired product Ms was detected. Additional one reaction was set up as described above and combined for purification. The combined reaction was diluted with water (150 mL) and extracted with ethyl acetate (3×100 mL). The combined organic layer were washed with brine (150 mL), dried over Na₂SO₄ and concentrated to give crude product. The crude product was purified by silica gel chromatography (eluting with 0 to 40% ethyl acetate in petroleum ether) to give (2.25 g, yield 46.23%) as a yellow solid.

¹H NMR (ET42365-329-P1A, 400 MHz, CHLOROFORM-d) δ −0.11 (s, 9H), 0.49-0.57 (m, 2H), 2.84-2.92 (m, 2H), 3.90 (s, 3H), 3.94 (s, 3H), 4.40 (s, 2H), 5.06 (s, 2H), 6.86 (s, 1H), 6.91 (d, J=8.50 Hz, 1H), 7.17 (d, J=8.25 Hz, 2H), 7.28-7.35 (m, 3H), 7.52-7.62 (m, 2H), 7.84 (s, 1H)

### General Procedure for Preparation of Compound 14-ET43259-333

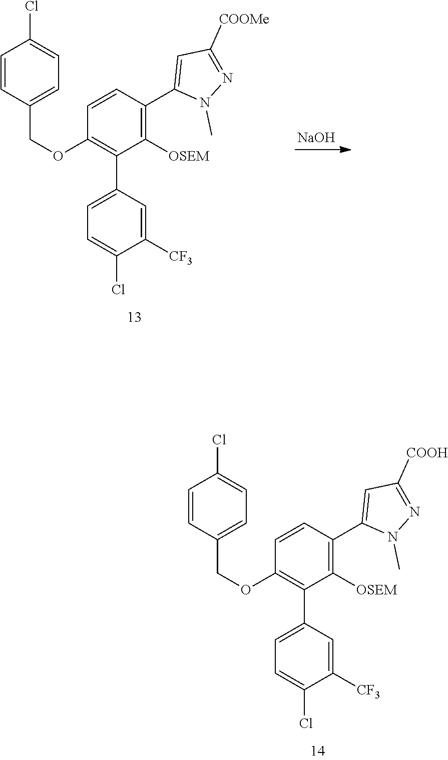

To a solution of Compound 13 (1.4 g, 2.05 mmol, 1 eq) in a mixture solution of THF (24 mL), MeOH (8 mL) and H₂O (8 mL) was added NaOH (123.24 mg, 3.08 mmol, 1.5 eq) at 0° C., the reaction was stirred at 25° C. for 3 hrs. LCMS showed the starting material was consumed and a new peak with desired product Ms was detected. The reaction was concentrated to give a residue, diluted with water (40 mL) and adjusted to pH=3 with 1 M HCL. The reaction was extracted with ethyl acetate (3×30 mL). The combined organic layer were washed with brine (50 mL), dried over Na₂SO₄ and concentrated to give Compound 14 (0.85 g, 1.27 mmol, 61.99% yield) as a yellow solid. The product was used to the next step directly without further purification.

¹H NMR (ET42365-333-P1A. 400 MHz, CHLOROFORM-d) δ −0.11 (s, 9H), 0.49-0.59 (m, 2H), 2.85-2.94 (m, 2H), 3.92 (s, 3H), 4.42 (s, 2H), 5.06 (s, 2H), 6.89-6.95 (m, 2H), 7.18 (d, J=8.50 Hz, 2H), 7.28-7.35 (m, 3H), 7.52-7.62 (m, 2H), 7.85 (d, J=1.63 Hz, 1H)

### General Procedure for Preparation of Compound 1 B-ET42365-349

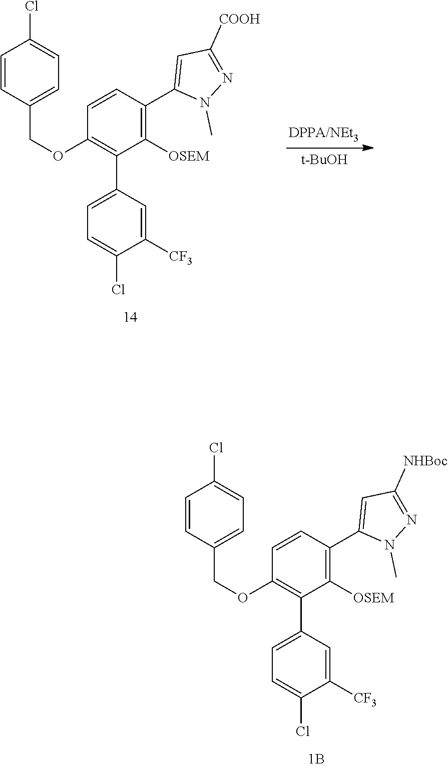

To a solution of Compound 14 (0.5 g, 748.98 μmol, 1 eq) in t-BuOH (50 mL) were added Et₃N (227.37 mg, 2.25 mmol, 312.75 μL, 3 eq) and DPPA (247.34 mg, 898.78 μmol, 194.76 μL, 1.2 eq), the reaction was stirred at 100° C. for 12 hrs. LCMS showed the starting material was consumed and a new peak with desired product Ms was detected. The reaction was concentrated to give crude product. The crude product was purified by silica gel chromatography (eluting with 0 to 50% ethyl acetate in petroleum ether) to give Compound 14 (0.22 g, yield 39.76%) as a yellow solid.

$$LCMS ( ESI + ) : RT = 1.153 min , m / z 738.2 {( M + H )}^{+} .$$

5-95AB 2 min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Compound 1 D-ET42365-353

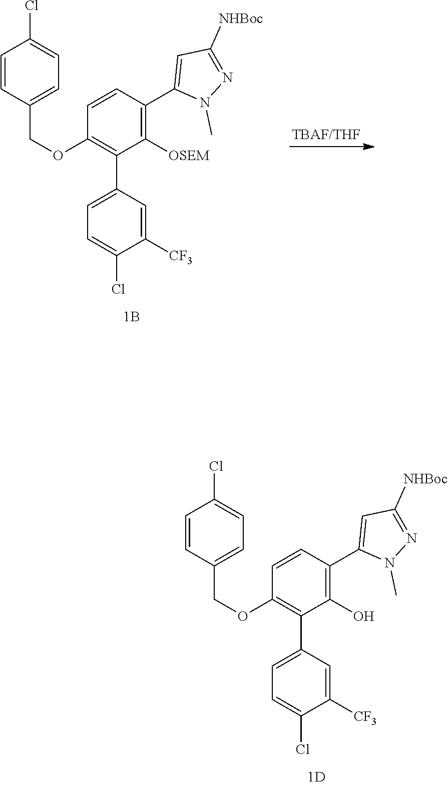

A mixture of Compound 1B (200 mg, 270.75 μmol, 1 eq) in TBAF (1 M, 4 mL, 14.77 eq) was stirred at 25° C. for 72 hrs. LCMS showed about 41% of the starting material was remaining and 32.5% of desired product was detected. The reaction was concentrated to give crude product. The crude product was purified by silica gel chromatography (eluting with 0 to 50% ethyl acetate in petroleum ether) to give Compound 1D (0.1 g, yield 48.56%) as a yellow solid.

$$LCMS ( ESI + ) : RT = 0.947 min , m / z 608.1 {( M + H )}^{+} .$$

5-95AB_2_min: LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Series 11-3-8_1C-ET42365-353

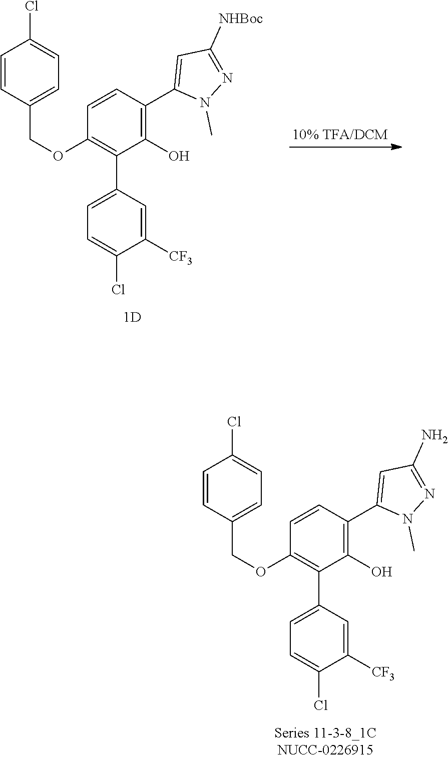 [SMILES: Cn1nc(NC(=O)OC(C)(C)C)cc1-c1ccc(OCc2ccc(Cl)cc2)c(-c2ccc(Cl)c(C(F)(F)F)c2)c1O ; Cn1nc(N)cc1-c1ccc(OCc2ccc(Cl)cc2)c(-c2ccc(Cl)c(C(F)(F)F)c2)c1O]

To a solution Compound 1D (90 mg, 147.92 μmol, 1 eq) in DCM (2 mL) was added TFA (168.66 mg, 1.48 mmol, 109.52 μL, 10 eq) dropwise at 0° C., the reaction was stirred at 25° C. for 12 hrs. LCMS showed the starting material was consumed and a new peak with desired product Ms was detected. The reaction was concentrated to give crude product, which was purified by Prep-HPLC (column: Phenomenex Luna 80*30 mm*3 um; mobile phase: [water (TFA)-ACN]; B %: 40%-70%, 8 min) to give Series 11-3-8_1C (8.1 mg, yield 10.77%) as a yellow solid.

¹H NMR (400 MHz, CHLOROFORM-d) δ 3.65 (s, 3H), 5.05 (s, 2H), 5.68 (s, 1H), 6.71 (d, J=8.63 Hz, 1H), 7.18 (dd, J=16.76, 8.50 Hz, 3H), 7.26-7.27 (m, 1H), 7.31 (d, J=8.50 Hz, 2H), 7.51-7.56 (m, 1H), 7.58-7.64 (m, 1H), 7.79 (d, J=1.63 Hz, 1H)

$$LCMS ( ESI + ) : RT = 2.718 min , m / z 507.9 {( M + H )}^{+} .$$

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

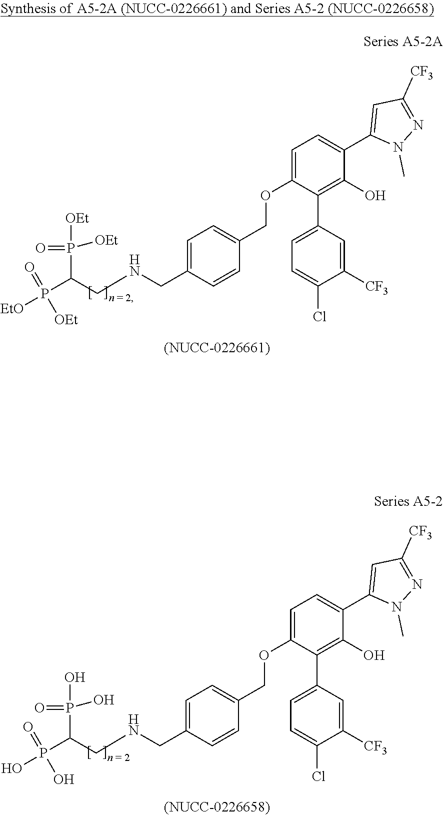

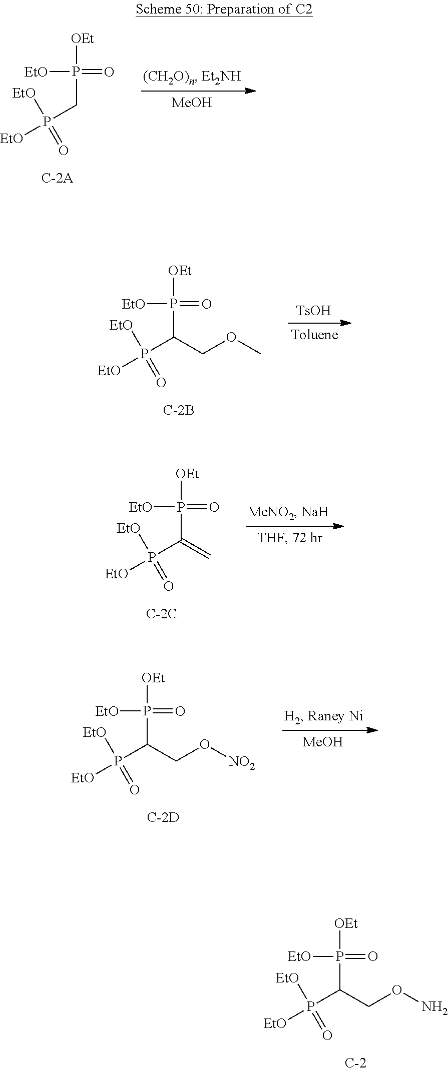

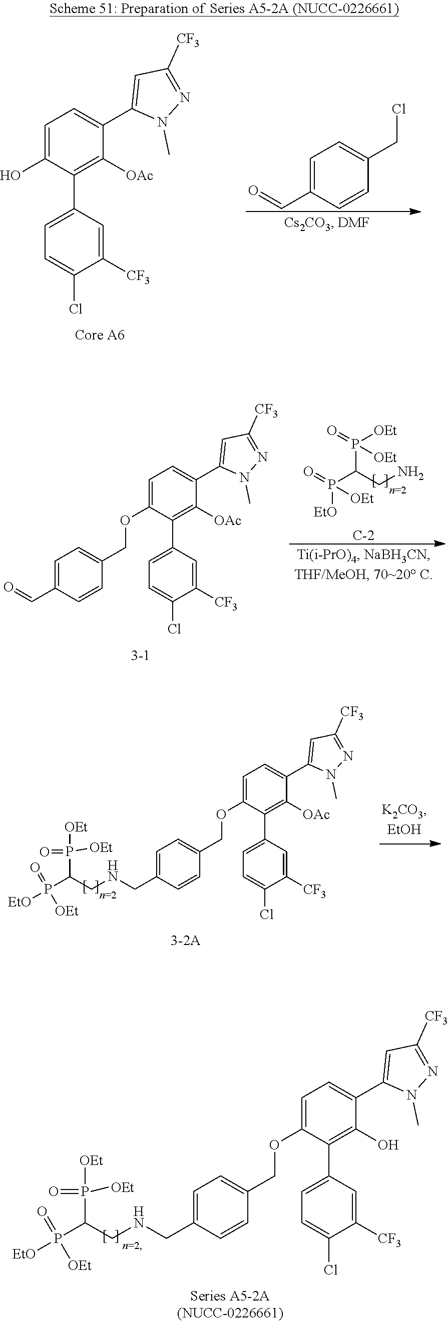

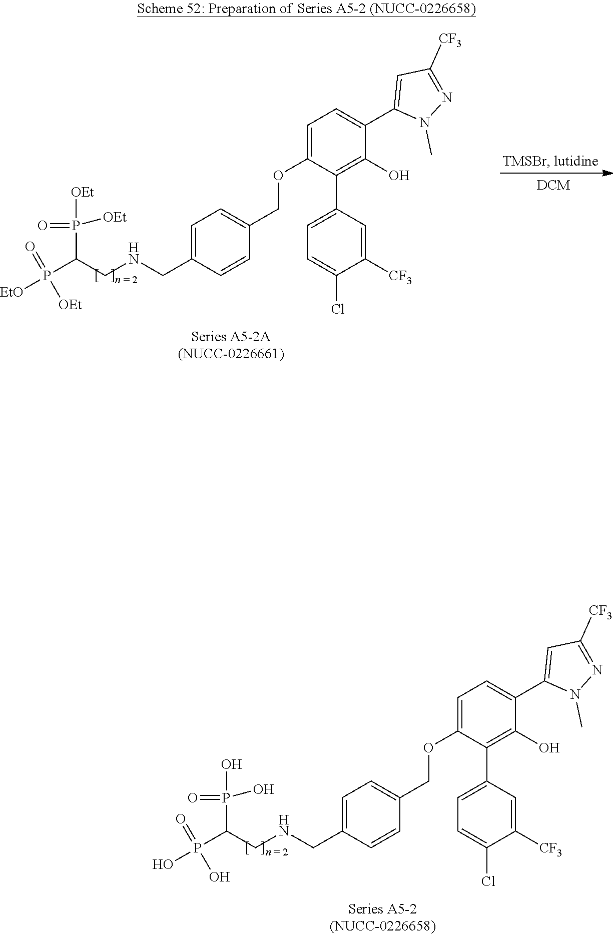

### Chemical Synthesis

### LCMS Methods:

### Method 1: 5_95AB_6min-220-254-ELSD

LC/MS (The gradient was 5% B in 0.40 min and 5.95% B at 0.40-3.00 min. hold on 95% B for 1.00 min. and then 95-5% B in 0.01 min. the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

### Experimentals for Largest Scale Run

### General Procedure for Preparation of Compound C-2B-ET43318-312

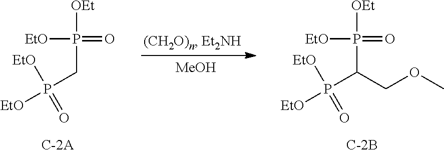

To a solution of formaldehyde (520.90 mg, 17.35 mmol) in MeOH (10 mL) was added N-ethylethanamine (279.13 mg, 3.82 mmol) dropwise at 25° C. The mixture was heated to 70° C. and stirred for 2 hrs. Then C-2A (1 g, 3.47 mmol) was added into and the resulting mixture was stirred for 16 hrs at 70° C. The mixture was concentrated under reduced pressure to give C-2B (1 g, 78.07% yield) as a colorless oil which was used directly for next step without further purification.

¹H NMR (400 MHz, CHLOROFORM-d) δ 1.34 (t, J=6.44 Hz, 12H), 2.62-2.77 (m, 1H), 3.37 (s, 3H), 3.89 (td, J=16.20, 5.50 Hz, 2H), 4.15-4.21 (m, 8H)

### General Procedure for Preparation of Compound C-2C-ET43318-325

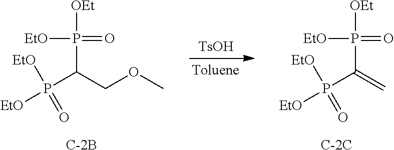

To a solution of C-2B (2 g, 6.02 mmol) in toluene (25 mL) was added TsOH (103.65 mg, 601.93 μmol) at 20° C. The mixture was heated to 110° C. and stirred for 16 hrs. TLC (ethyl acetate/MeOH=0/1) showed the starting material was consumed and new spot generated. Product with desired Ms was detected by LCMS. The mixture was concentrated under high vacuum to give a residue. The residue was purified by column chromatography on silica gel (eluting with ethyl acetate/methanol=1/0 to 10/1) to give C-2C (1 g, 49.80% yield) as a colorless oil.

¹H NMR (400 MHz, CHLOROFORM-d) δ 1.36 (t, J=7.07 Hz, 12H), 4.07-4.24 (m, 8H), 6.87-7.11 (m, 2H)

### General Procedure for Preparation of Compound C-2D-ET43318-236

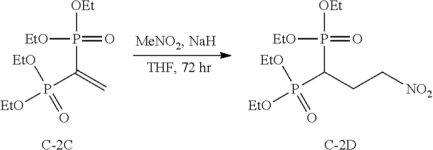

NaH (66.61 mg, 1.67 mmol) was added to anhydrous THF (30 mL) at 20° C. Then nitromethane (11.81 g, 193.52 mmol) was added dropwise to the stirred suspension. After 1 hour, C-2C (1 g, 3.33 mmol) was added in one portion and the mixture was stirred for 72 hours at 20° C. TLC (ethyl acetate/MeOH=5/1, Rf=0.6) showed the starting material was consumed and new spot generated. The reaction mixture was poured into saturated NH₄Cl solution (100 mL) and extracted with ethyl acetate (2×30 mL). The organic layers were combined, washed with brine (2×30 mL), dried over Na₂SO₄, filtered and concentrated under reduced pressure to give a residue. The residue was purified by column chromatography on silica gel (eluting with ethyl acetate/methanol=1/0 to 10/1) to give C-2D (1 g, 74.79% yield) as a yellow oil.

¹H NMR (400 MHz, CHLOROFORM-d) $1.36 (td, J=7.07, 0.63 Hz, 12H), 2.42-2.66 (m, 3H), 4.14-4.27 (m, 8H), 4.71 (t, J=6.88 Hz, 2H)

### General Procedure for Preparation of Compound C-2-ET43318-240

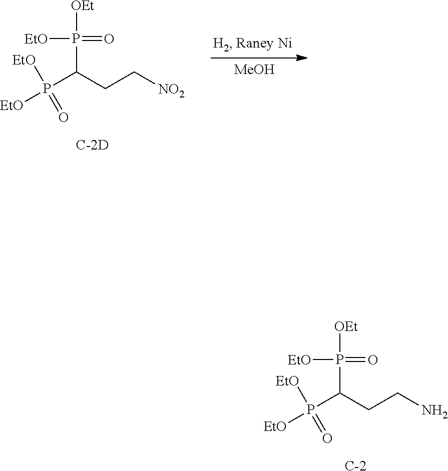

To a mixture of Raney-Ni (142.29 mg, 1.66 mmol) in MeOH (30 mL) was added C-2D (600 mg, 1.66 mmol) at 20° C. The mixture was stirred for 12 hrs under hydrogen atmosphere (15 psi). TIC (ethyl acetate/MeOH=5/1) showed the starting material was consumed and new spot generated. Product with desired Ms was detected by LCMS. The mixture was filtered through celite and the filtrate was concentrated under reduced pressure to give C-2 (500 mg, 81.79% yield) as a yellow oil which was used directly for next step without purification.

¹H NMR (400 MHz, CHLOROFORM-d) δ 1.34 (t, J=7.07 Hz, 12H), 1.97-2.11 (m, 2H), 2.45-2.61 (m, 1H), 2.91 (t, J=7.00 Hz, 2H), 4.13-4.23 (m, 8H)

### General Procedure for Preparation of Compound 3-1-ET43318-237

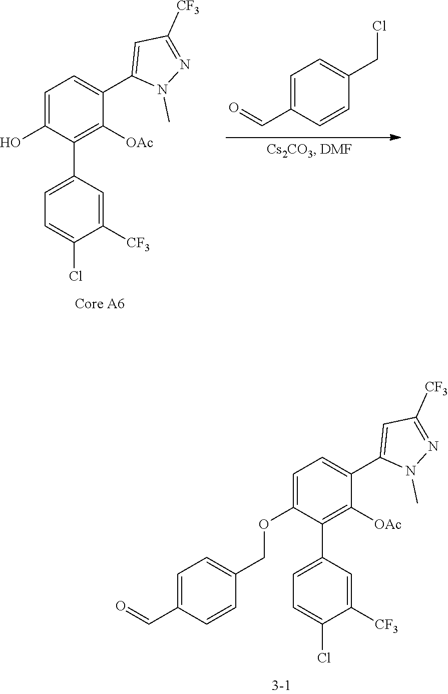 [SMILES: CC(=O)Oc1c(-c2cc(C(F)(F)F)nn2C)ccc(O)c1-c1ccc(Cl)c(C(F)(F)F)c1 ; O=Cc1ccc(CCl)cc1 ; CC(=O)Oc1c(-c2cc(C(F)(F)F)nn2C)ccc(OCc2ccc(C=O)cc2)c1-c1ccc(Cl)c(C(F)(F)F)c1]

To a solution of 4-(chloromethyl)benzaldehyde (322.90 mg, 2.09 mmol), Core A6 (1 g, 2.09 mmol) in DMF (5 mL) was added Cs₂CO₃ (680.53 mg, 2.09 mmol) at 20° C. The mixture was stirred for 12 hrs. LCMS showed 21% of the starting material was remaining and 49.2% peak (Rt=0.900 min) with desired Ms was detected. The reaction mixture was poured into water (100 mL) and extracted with ethyl acetate (30 mL×3). The organic layers were combined, washed with brine (2×10 mL), dried over Na₂SO₄, filtered and concentrated under reduced pressure to give a residue. The residue was purified by column chromatography on silica gel (eluting with petroleum ether/ethyl acetate=20/1 to 3/1) to give 3-1 (600 mg, 48.1% yield) as a white solid.

¹H NMR (400 MHz, CHLOROFORM-d) δ 1.71 (s, 3H), 3.74 (s, 3H), 5.13 (s, 2H), 6.42-6.44 (m, 1H), 6.71 (d, J=8.00 Hz, 1H), 6.98 (d, J=8.63 Hz, 1H), 7.25-7.29 (m, 1H), 7.32 (br d, J=8.00 Hz, 2H), 7.37-7.43 (m, 1H), 7.60 (d, J=8.00 Hz, 1H), 7.68 (d, J=1.63 Hz, 1H), 7.78-7.79 (m, 1H), 7.78 (s, 1H), 9.94 (s, 1H)

### General Procedure for Preparation of Compound 3-2A-ET43318-255

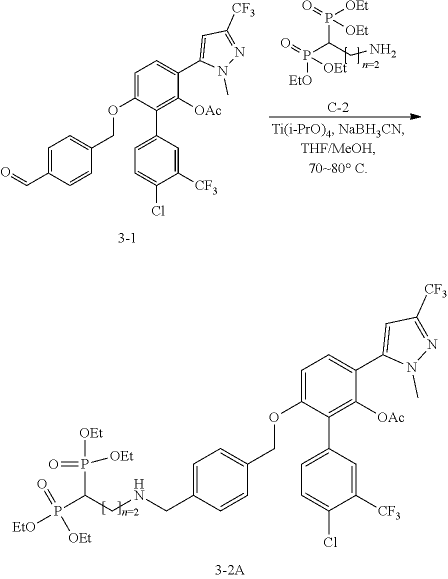

To a solution of C-2 (208.12 mg, 628.24 μmol) in anhydrous MeOH (2 mL) and THF (2 mL) was added 3-1 (250 mg, 418.83 μmol) and Ti(i-PrO)4 (297.59 mg, 1.05 mmol) dropwise at 20° C. The mixture was heated to 70° C. and stirred for 12 hrs. After the mixture was cooled to 20° C., NaBH₃CN (78.96 mg, 1.26 mmol) was added into at 20° C. The resulting reaction mixture was stirred at 20° C. for 6 hrs. LCMS showed the starting material was consumed and 34.9% peak (Rt=0.772 min) with desired Ms was detected. TLC (ethyl acetate/MeOH=3/1) showed new spot generated. The reaction mixture was diluted with ethyl acetate (30 mL), poured into H₂O (60 mL), filtered. The filtrate was extracted with ethyl acetate (3×20 mL). The organic layers were combined, washed with brine (2×10 mL), dried over Na₂SO₄, filtered and concentrated under reduced pressure to give a residue. The residue was purified by column chromatography on silica gel (eluting with ethyl acetate/MeOH=20/1 to 5/1) to give 3-2A (300 mg, 38.87% yield) as a yellow oil.

¹H NMR (400 MHz, CHLOROFORM-d) δ 1.32 (t, J=7.13 Hz, 12H), 2.03 (s, 3H), 2.16-2.23 (m, 2H), 2.56-2.67 (m, 1H), 2.98 (br t, J=6.63 Hz, 2H), 3.80-3.89 (m, 5H), 4.17 (br d, J=6.50 Hz, 8H), 5.11 (s, 2H), 6.45-6.53 (m, 1H), 7.06 (d, J=8.63 Hz, 1H), 7.19-7.23 (m, 2H), 7.30-7.39 (m, 3H), 7.43-7.57 (m, 2H), 7.73 (s, 1H)

### General Procedure for Preparation of Compound Series A5-2A (NUCC-0226661)-ET43318-260

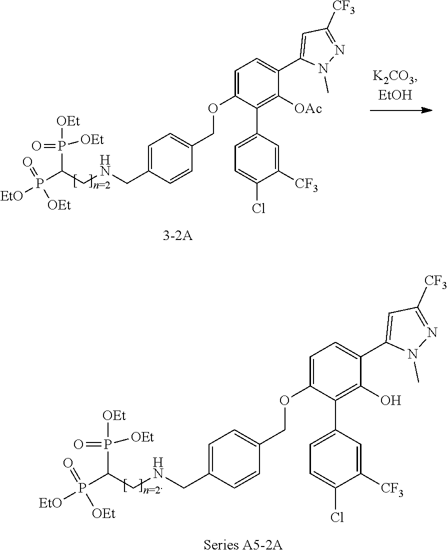

To a solution of 3-2A (290 mg, 317.92 μmol) in MeOH (4 mL) was added K₂CO₃ (131.82 mg, 953.75 μmol) at 20° C.′. The mixture was stirred for 12 hrs. LCMS showed the starting material was consumed and 74.3% peak (Rt=0.747 min) with desired Ms was detected. The mixture was filtered to give a filtrate. The filtrate was purified by prep-HPLC to give Series A5-2A (80 mg, 28.25% yield) as an off-white solid.

Method of prep-HPLC:
Instrument: Gilson 281 semi-preparative HPLC system
Mobile phase: A: 10 mM NH₄HCO₃ in H₂O; B: Acetonitrile
Column: Waters Xbridge BEH C18 100*30 mm*10 um
Flow rate: 25 mL/min
Monitor wavelength: 220&254 nm

| Time | B % |
|---|---|
| 0.0 | 45 |
| 10.0 | 75 |
| 10.1 | 75 |
| 10.2 | 100 |
| 13.2 | 100 |
| 13.3 | 45 |
| 14.5 | 45 |

¹H NMR (400 MHz, CHLOROFORM-d) δ 1.31 (td, J=7.07, 1.75 Hz, 12H), 2.07-2.20 (m, 2H), 2.55-2.71 (m, 1H), 2.87 (br t, J=6.69 Hz, 2H), 3.78 (s, 2H), 3.84 (s, 3H), 4.11-4.20 (m, 8H), 5.07 (s, 2H), 6.56 (s, 1H), 6.75 (d, J=8.63 Hz, 1H), 7.15-7.23 (m, 3H), 7.30 (d, J=8.00 Hz, 2H), 7.51-7.56 (m, 1H), 7.57-7.63 (m, 1H), 7.80 (d, J=1.63 Hz, 1H)

$$LCMS ( ESI + ) : RT = 2.506 min , m / z 870.1 {( M + H )}^{+} .$$

### 5_95AB_6min-220-254-ELSD

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (Sum particles). Detection methods are diode array (DAD) and evaporative light scattering (ELSD) detection as well as positive electrospray ionization. MS range was 100-1000.

### General Procedure for Preparation of Series A5-2 (NUCC-0226658)-ET43318-278

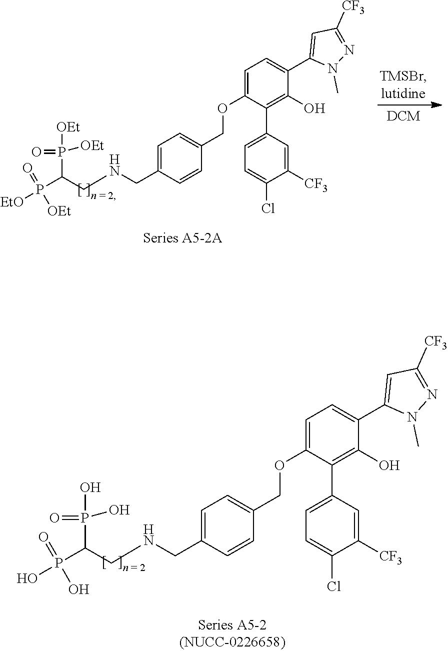

To a solution of Series A5-2A (50 mg, 57.46 μmol), 2,6-dimethylpyridine (184.71 mg, 1.72 mmol) in DCM (0.2 mL) was added TMSBr (263.90 mg, 1.72 mmol) dropwise at 0° C. The mixture was warmed up to 20° C. and stirred for 48 hrs. LCMS showed the starting material was consumed and 14.6% product (Rt=0.751 min) with desired Ms was detected. The mixture was concentrated under reduced pressure to give a residue. The residue was purified by prep-HPLC and lyophilized to give Series A5-2 (15 mg, 29.58% yield) as a white solid.

Method of prep-HPLC:
Instrument: Gilson 281 semi-preparative HPLC system
Mobile phase: A: TFA/H₂O = 0.075% v/v; B: Acetonitrile
Column: Phenomenex Luna 80*30 mm*3 um
Flow rate: 25 mL/min
Monitor wavelength: 220&254 nm

| Time | B % |
|---|---|
| 0.0 | 20 |
| 8.0 | 75 |
| 8.1 | 75 |
| 8.2 | 100 |
| 11.2 | 100 |
| 11.3 | 20 |
| 12.5 | 20 |

¹H NMR (400 MHz, DMSO-d₆) δ 1.94-2.20 (m, 3H), 3.15 (br s, 2H), 3.75 (s, 3H), 4.05 (br s, 2H), 5.14 (br s, 2H), 6.72 (s, 1H), 6.87 (br d, J=8.50 Hz, 1H), 7.23-7.36 (m, 3H), 7.43 (br s, 2H), 7.66-7.72 (m, 1H), 7.73-7.80 (m, 1H), 7.85 (s, 1H)

$$LCMS ( ESI + ) : RT = 2.439 min , m / z 758. {( M + H )}^{+} .$$

### 5_95CD_6min-220-254-ELSD

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

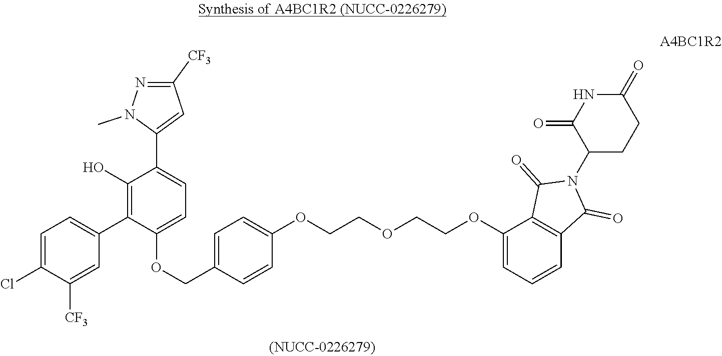 [SMILES: Cn1nc(C(F)(F)F)cc1-c1ccc(OCc2ccc(OCCOCCOc3cccc4c3C(=O)N(C3CCC(=O)NC3=O)C4=O)cc2)c(-c2ccc(Cl)c(C(F)(F)F)c2)c1O]

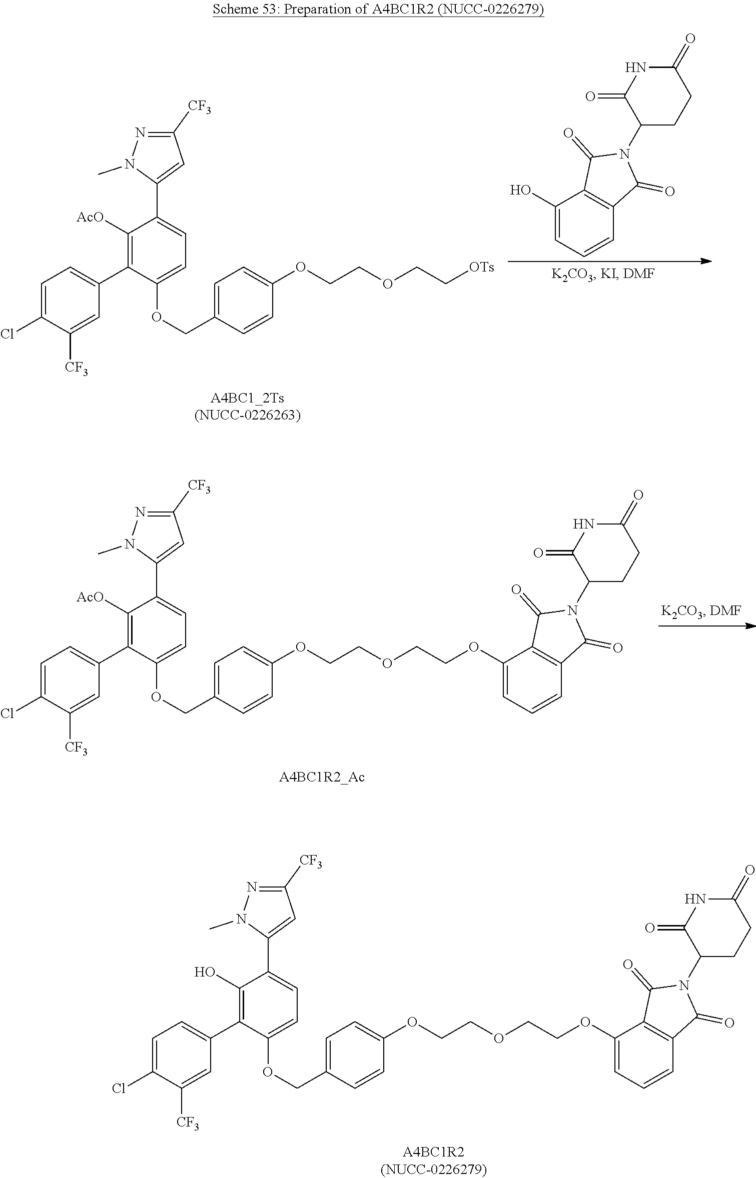 [SMILES: CC(=O)Oc1c(-c2cc(C(F)(F)F)nn2C)ccc(OCc2ccc(OCCOCCOS(=O)(=O)c3ccc(C)cc3)cc2)c1-c1ccc(Cl)c(C(F)(F)F)c1 ; O=C1CCC(N2C(=O)c3cccc(O)c3C2=O)C(=O)N1 ; CC(=O)Oc1c(-c2cc(C(F)(F)F)nn2C)ccc(OCc2ccc(OCCOCCOc3cccc4c3C(=O)N(C3CCC(=O)NC3=O)C4=O)cc2)c1-c1ccc(Cl)c(C(F)(F)F)c1 ; Cn1nc(C(F)(F)F)cc1-c1ccc(OCc2ccc(OCCOCCOc3cccc4c3C(=O)N(C3CCC(=O)NC3=O)C4=O)cc2)c(-c2ccc(Cl)c(C(F)(F)F)c2)c1O]

### LCMS Methods:

### Method 1: 5_95AB_6min-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization.

### Method 2:5-95AB_2_min

LC/MS (The column used for chromatography was a Kinetex 5 μm EVO C18 100A 2.1*30 mm. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

### Method 3: 5_95CD_6min-220-254-ELSD

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge Shield RP18 2.1*50 mm column (5 um particles). Detection methods are diode array (DAD) and evaporative light scattering (ELSD) detection as well as positive electrospray ionization.

### Experimentals for Largest Scale Run

### General Procedure for Preparation of A4BC1R²_Ac-ET32240-1224

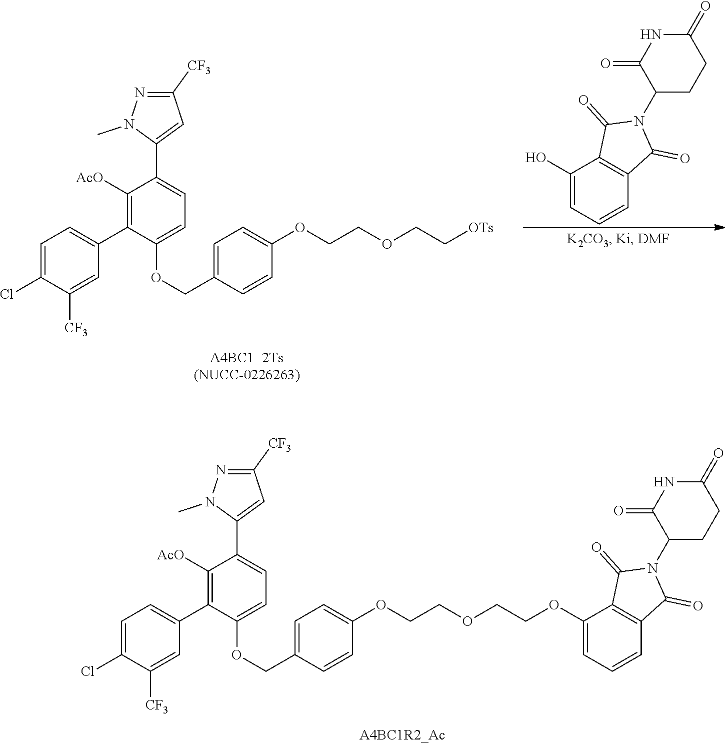 [SMILES: CC(=O)Oc1c(-c2cc(C(F)(F)F)nn2C)ccc(OCc2ccc(OCCOCCOS(=O)(=O)c3ccc(C)cc3)cc2)c1-c1ccc(Cl)c(C(F)(F)F)c1 ; O=C1CCC(N2C(=O)c3cccc(O)c3C2=O)C(=O)N1 ; CC(=O)Oc1c(-c2cc(C(F)(F)F)nn2C)ccc(OCc2ccc(OCCOCCOc3cccc4c3C(=O)N(C3CCC(=O)NC3=O)C4=O)cc2)c1-c1ccc(Cl)c(C(F)(F)F)c1]

To a solution of A4BC1 (80 mg, 96.71 μmol, 1 eq) in DMF (1 mL) was added 2-(2,6-dioxo-3-piperidyl)-4-hydroxy-isoindoline-1,3-dione (29.17 mg, 106.38 μmol, 1.1 eq), K₂CO₃ (53.47 mg, 386.85 μmol, 4 eq) and KI (1.61 mg, 9.67 μmol, 0.1 eq). The mixture was stirred at 50° C. for 12 hrs. LCMS showed all starting materials consumed and desired product was detected. The reaction mixture was quenched with water (10 mL) and extracted with EtOAc (3×10 mL). The organic layer was concentrated to give crude product. The residue was purified by prep-TLC (hexane/EtOAc=1:1) to give A4BC1R2_Ac (60 mg, 64.57 μmol, 66.77% yield) as a white solid.

$$LCMS ( ESI + ) : m / z = 929.3 ( M + H ) + , RT : 0.893 min .$$

### Method 2:5-95AB_2_min

LC/MS (The column used for chromatography was a Kinetex 5 μm EVO C18 100A 2.1*30 mm. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 mL/min

### General Procedure for Preparation of A4BC1R² (NUCC-0226279)-ET32240-1226, 1280

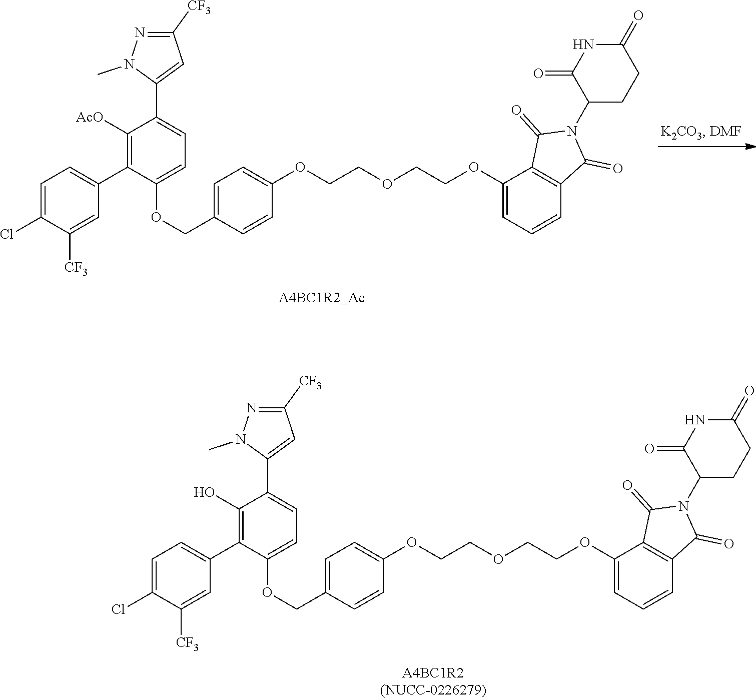 [SMILES: CC(=O)Oc1c(-c2cc(C(F)(F)F)nn2C)ccc(OCc2ccc(OCCOCCOc3cccc4c3C(=O)N(C3CCC(=O)NC3=O)C4=O)cc2)c1-c1ccc(Cl)c(C(F)(F)F)c1 ; Cn1nc(C(F)(F)F)cc1-c1ccc(OCc2ccc(OCCOCCOc3cccc4c3C(=O)N(C3CCC(=O)NC3=O)C4=O)cc2)c(-c2ccc(Cl)c(C(F)(F)F)c2)c1O]

To a solution of A4BC1R²_Ac (50 mg, 53.81 μmol, 1 eq) in DMF (1 mL) was added K₂CO₃ (22.31 mg, 161.43 μmol, 3 eq). The mixture was stirred at 80° C. for 12 hrs. LCMS showed all starting materials consumed and desired product was detected. The reaction mixture was quenched with water (10 mL) and extracted with EtOAc (3×10 mL). The organic layer was concentrated to give crude product. The residue was purified by prep-HPLC (TFA condition, column: Phenomenex Luna C18 100*30 mm*5 um; mobile phase: [water (0.1% TFA)-ACN]; B %: 55%-85%, 10 min) to give A4BC1R² (NUCC-0226279) (14 mg, 15.46 μmol, 28.74% yield, 98% purity, TFA salt) as a white solid.

The TFA salt product was further purified by prep-HPLC (neutral condition, column: Phenomenex Gemini-NX C18 75*30 mm*3 um; mobile phase: [water (10 mM NH₄HCO₃)-ACN]; B %: 45%-65%, 8 min) to give A4BC1R² (NUCC-0226279) (5 mg, 5.58 μmol, 35.36% yield, 99% purity) as a white solid.

### TFA Salt:

¹H NMR (400 MHz, CDCl₃) δ 2.12-2.14 (m, 1H), 2.75-2.87 (m, 4H), 3.87 (s, 3H), 4.00-4.14 (m, 4H), 4.13-4.14 (m, 2H), 4.37-4.39 (m, 2H), 4.93-4.97 (m, 2H), 5.02 (s, 1H), 6.57 (s, 1H), 6.77 (d, J=8.68 Hz, 1H), 6.87 (d, J=8.56 Hz, 2H), 7.14 (d, J=8.56 Hz, 2H), 7.21 (d, J=8.56 Hz, 1H), 7.29 (br s, 1H), 7.46 (d, J=7.21 Hz, 1H), 7.50-7.55 (m, 1H), 7.57-7.68 (m, 2H), 7.78 (s, 1H), 8.06 (s, 1H)

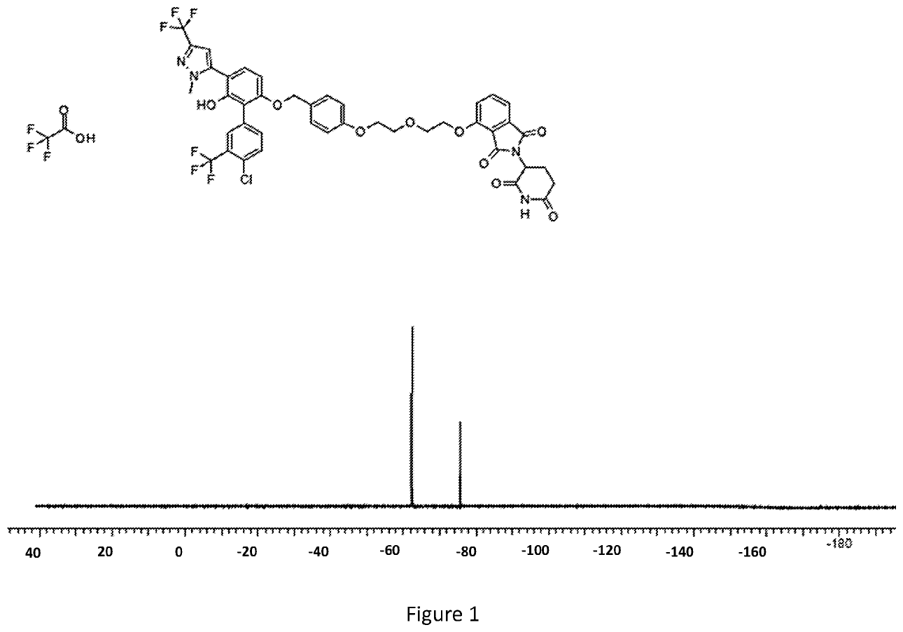

For ¹⁹F NMR of A4BC1R², see FIG. 1.

$$LCMS ( ESI + ) : m / z = 887. ( M + H ) + , RT : 2.995 min .$$

### 5_95AB_6min-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization

### Free Base:

¹H NMR (400 MHz, CHLOROFORM-d) δ 2.07-2.16 (m, 1H), 2.68-2.94 (m, 3H), 3.85 (s, 3H), 3.98-4.05 (m, 4H), 4.11-4.16 (m, 2H), 4.36-4.41 (m, 2H), 4.95 (dd, J=12.23, 5.26 Hz, 1H), 5.02 (s, 2H), 5.12 (s, 1H), 6.57 (s, 1H), 6.77 (d, J=8.68 Hz, 1H), 6.87 (d, J=8.56 Hz, 2H), 7.14 (d, J=8.56 Hz, 2H), 7.21 (d, J=8.56 Hz, 1H), 7.29 (br s, 1H), 7.46 (d, J=7.21 Hz, 1H), 7.50-7.55 (m, 1H), 7.57-7.68 (m, 2H), 7.78 (d, J=1.71 Hz, 1H), 7.93 (br s, 1H)

$$LCMS ( ESI + ) : m / z = 887. ( M + H ) + , RT : 3.502 min .$$

### 5_95CD_6min-220-254-ELSD

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge Shield RP18 2.1*50 mm column (5 um particles). Detection methods are diode array (DAD) and evaporative light scattering (ELSD) detection as well as positive electrospray ionization. MS range was 100-1000.

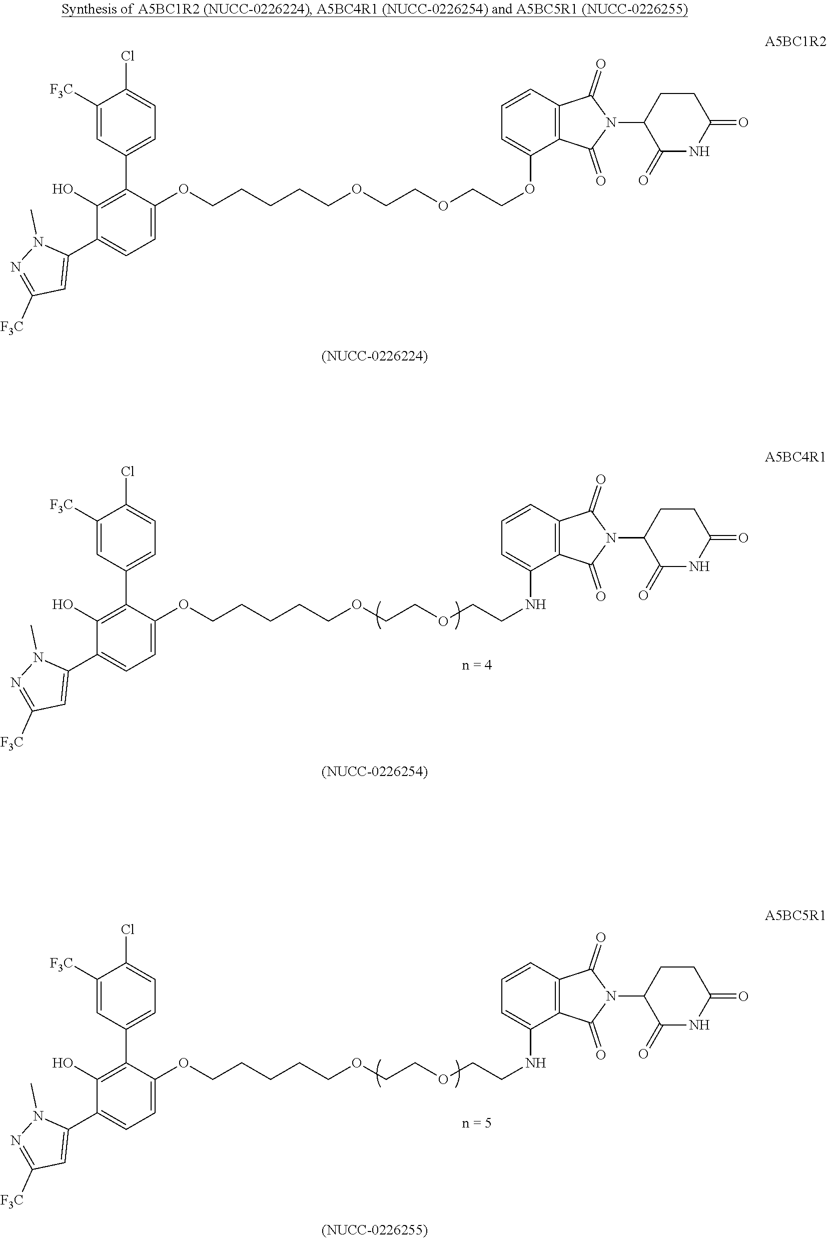

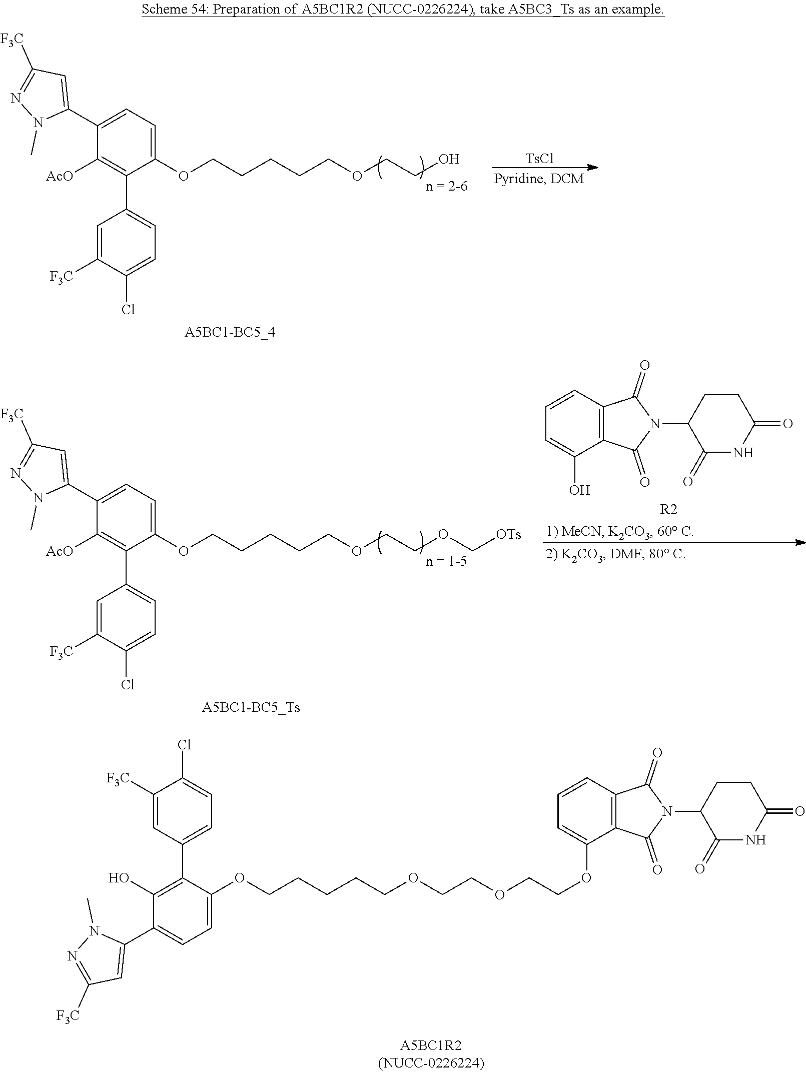

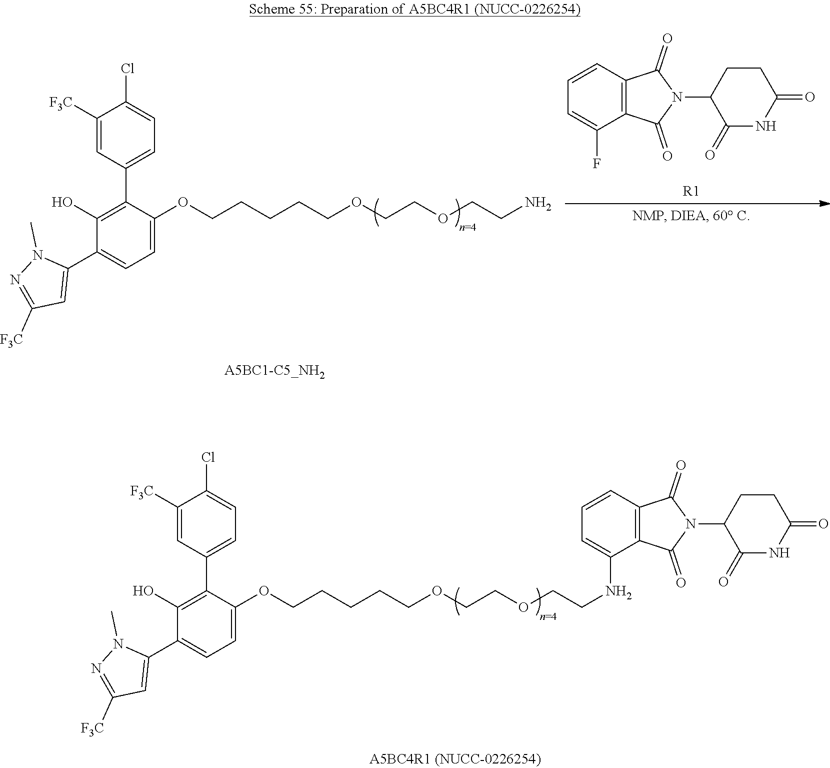

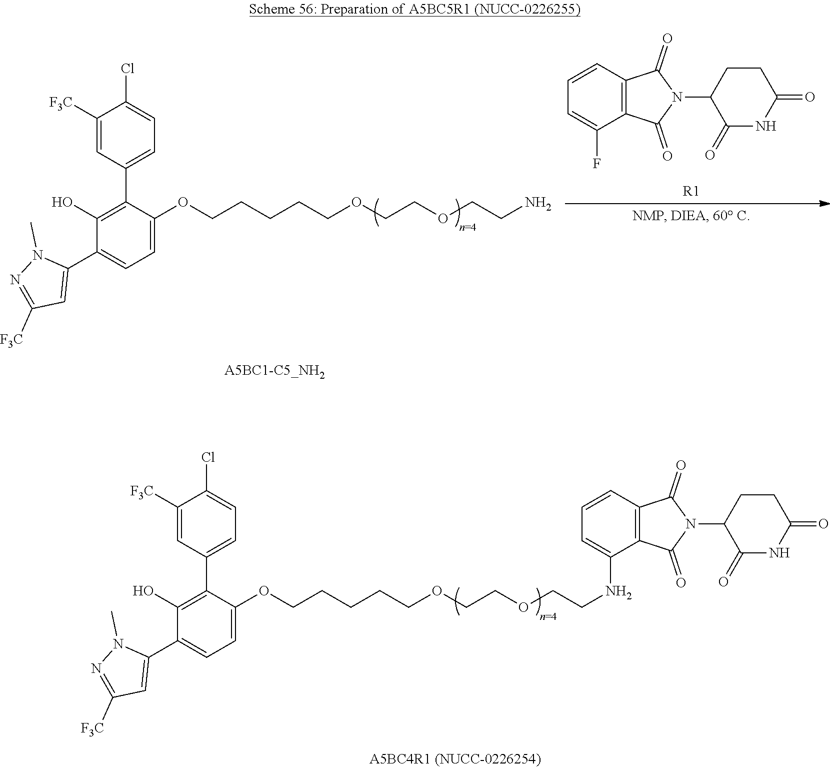

### LCMS Methods:

### Method 1: 5_95AB_6min-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization.

### Method 2: 5_95CD_6min-220-254-ELSD

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was a Xbridge Shield RP18 2.1*50 mm column (5 um particles). Detection methods are diode array (DAD) and evaporative light scattering (ELSD) detection as well as positive electrospray ionization.

### Method 3:5-95AB_2_min

LC/MS (The column used for chromatography was a Kinetex 5 um EVO C18 100A 2.1*30 mm. Detection methods are diode array (DAD). MS mode was positive electrospray ionization. MS range was 100-1000. Mobile phase A was 0.04% Trifluoroacetic acid in water, and mobile phase B was 0.02% Trifluoroacetic acid in HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

Method 4: 5_95CD_6 min_MS1500-220-254-ELSD

LC/MS (The gradient was 5% B in 0 40 min and 5-95% B at 0.40-3.40 min, hold on 95% B for 0.45 min, and then 95-5% B in 0.01 min, the flow rate was 0.8 ml/min. Mobile phase A was H2O+10 mM NH₄HCO₃, mobile phase B was Acetonitrile. The column used for chromatography was a Xbridge C18 2.1*50 mm column (Sum particles). Detection methods are diode array (DAD) and evaporative light scattering (ELSD) detection. MS mode was positive electrospray ionization.

### Method 5: 5_95AB_6min_MS1500-220-254-ELSD

LC/MS (The gradient was 5% B in 0.40 min and 5-95% B at 0.40-3.00 min, hold on 95% B for 1.00 min. and then 95-5% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Luna C18 50*2.0 mm column (5 um particles). Detection methods are diode array (DAD) and evaporative light scattering (ELSD) detection. MS mode was positive electrospray ionization.

### Experimentals for Largest Scale Run

### General Procedure for Preparation of A5BC3_Ts-ET37412-33

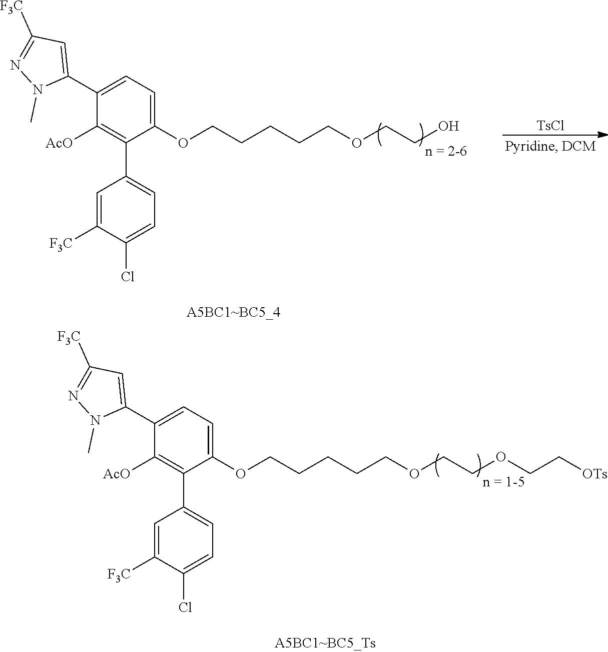

To a mixture of A5BC3_4 (40 mg, 53.97 μmol, 1 eq) and pyridine (42.69 mg, 539.73 μmol, 10 eq) in DCM (1 mL) was added TosCl (102.90 mg, 539.73 μmol, 10 eq) in one portion at 25° C. The reaction was stirred at 25° C. for 12 hr. TLC showed the reaction was completed. The reaction was concentrated to give a residue which was purified by Prep-TLC (PE:EA=1:1) to give A5BC3_Ts (40 mg, 44.68 μmol, 83% yield) as colorless oil.

### General Procedure for Preparation of A5BC1R2-ET37412-49

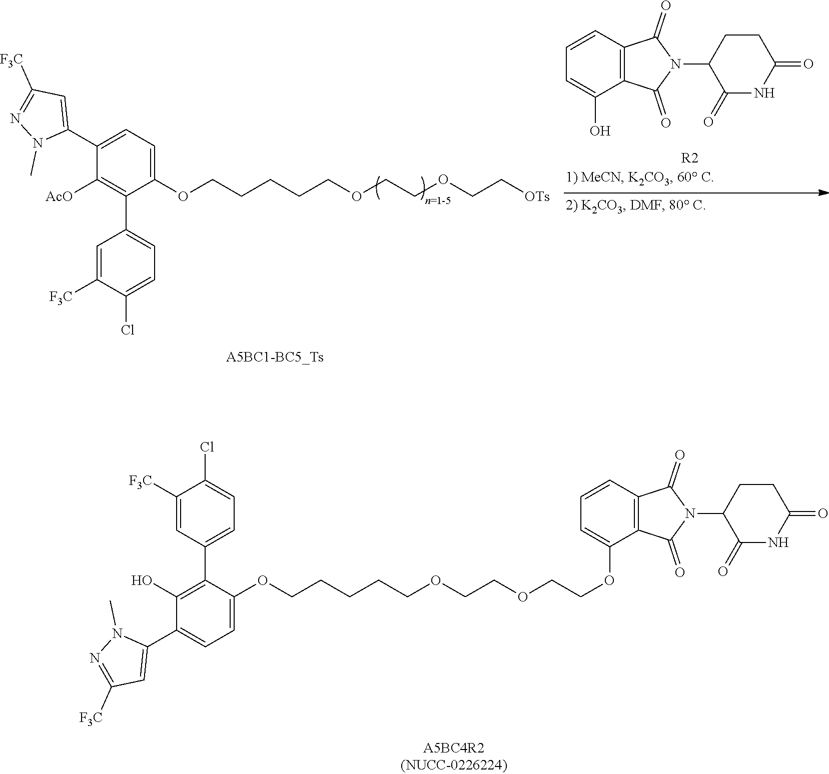

To a mixture of A5BC1_Ts (90 mg, 111.50 μmol, 1 eq) and R2 (45.86 mg, 167.25 μmol, 1.5 eq) in DMF (I mL) was added K₂CO₃ (30.82 mg, 222.99 μmol, 2 eq) and KI (1.85 mg, 11.15 μmol, 0.1 eq) in one portion at 25° C. The reaction was stirred at 60° C. for 12 h. LCMS showed desired product could be detected. To the reaction was added K₂CO₃ (13.68 mg, 98.99 μmol, 2 eq). The reaction was stirred at 80° C. for 12 hrs. LCMS showed all starting materials was consumed and desired product was detected. The reaction was filtered to give a residue which was purified by Prep-HPLC (column: Phenomenex luna C18 100*40 mm*5 um; mobile phase: [water (0.1% TFA)-ACN]; B %: 40%-80%, 8 min) to give A5BC1R2 (4 mg, 4.08 μmol, 8.24% yield, 100% purity, TFA) as white solid.

¹H NMR (ET37412-49-PIB, 400 MHz, CHLOROFORM-d) δ 1.33-1.41 (m, 2H), 1.63-1.71 (m, 4H), 1.63-1.69 (m, 1H), 2.13 (dd, J=7.76, 5.20 Hz, 1H), 2.71-2.94 (m, 3H), 3.41 (t, J=6.60 Hz, 2H), 3.56-3.61 (m, 2H), 3.77 (dd, J=5.50, 3.79 Hz, 2H), 3.84 (s, 3H), 3.92-4.01 (m, 4H), 4.32-4.38 (m, 2H), 4.94 (dd, J=12.23, 5.38 Hz, 1H), 6.57 (s, 1H), 6.66 (d, J=8.68 Hz, 1H), 7.20 (d, J=8.56 Hz, 1H), 7.28 (br s, 1H), 7.32 (br s, 1H), 7.47 (d, J=7.21 Hz, 1H), 7.53 (dd, J=5.69, 2.63 Hz, 1H), 7.60-7.70 (m, 2H), 7.76 (s, 1H), 7.91 (s, 1H)

$$LSMS ( ESI + ) : RT = 3.003 min , m / z 867. {( M + 1 )}^{+} .$$

LC/MS (The gradient was 5% B in 0.40 min and 5-95% B at 0.40-3.00 min. hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization.

### General Procedure for Preparation of A5BC4R1-ET37412-59

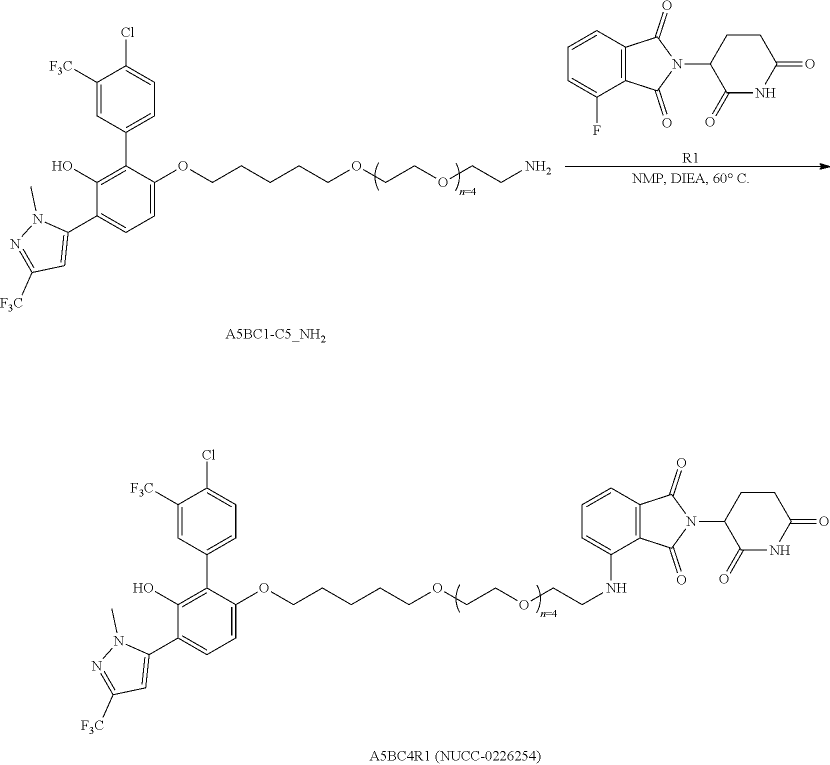

To a mixture of A5BC4_NH₂ (0.04 g, 53.90 μmol, 1 eq) in NMP (1 mL) was added DIEA (20.90 mg, 161.69 μmol, 28.16 μL, 3 eq) and R1 (16.38 mg, 59.29 μmol, 1.1 eq) in one portion at 20° C. The mixture was stirred at 20° C. for 30 min, then heated to 60° C. and stirred for 12 hours. LC-MS showed the reaction was completed. The reaction was filtered to give a residue which was purified by Prep-HPLC (column: Phenomenex Gemini-NX 150*30 mm*5 um; mobile phase: [water (0.1% TFA)-ACN]; B %: 55%-85%, 9 min) to give A5BC4R1 (16 mg, 30% yield) as yellowish oil.

¹H NMR (400 MHz, CHLOROFORM-d) δ 1.29-1.43 (m, 2H), 1.46-1.59 (m, 3H), 1.63-1.72 (m, 4H), 1.98-2.45 (m, 1H), 2.64-2.98 (m, 3H), 3.30-3.50 (m, 4H), 3.54-3.74 (m, 17H), 3.84 (s, 3H), 3.98 (t, J=6.17 Hz, 2H), 4.85-4.95 (m, 1H), 6.57 (s, 1H), 6.67 (d, J=8.68 Hz, 1H), 6.92 (d, J=8.44 Hz, 1H), 7.10 (d, J=7.09 Hz, 1H), 7.20 (d, J=8.56 Hz, 1H), 7.46-7.57 (m, 2H), 7.59-7.64 (m, 1H), 7.77 (s, 1H), 8.25 (br s, 1H)

$$LCMS ( ESI + ) : RT = 3.071 min , m / z 998.1 {( M + 1 )}^{+} .$$

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Luna C18 50*2.0 mm column (Sum particles). Detection methods are diode array (DAD) and evaporative light scattering (ELSD) detection. MS mode was positive electrospray ionization.

### General Procedure for Preparation of Target E-ET37412-28

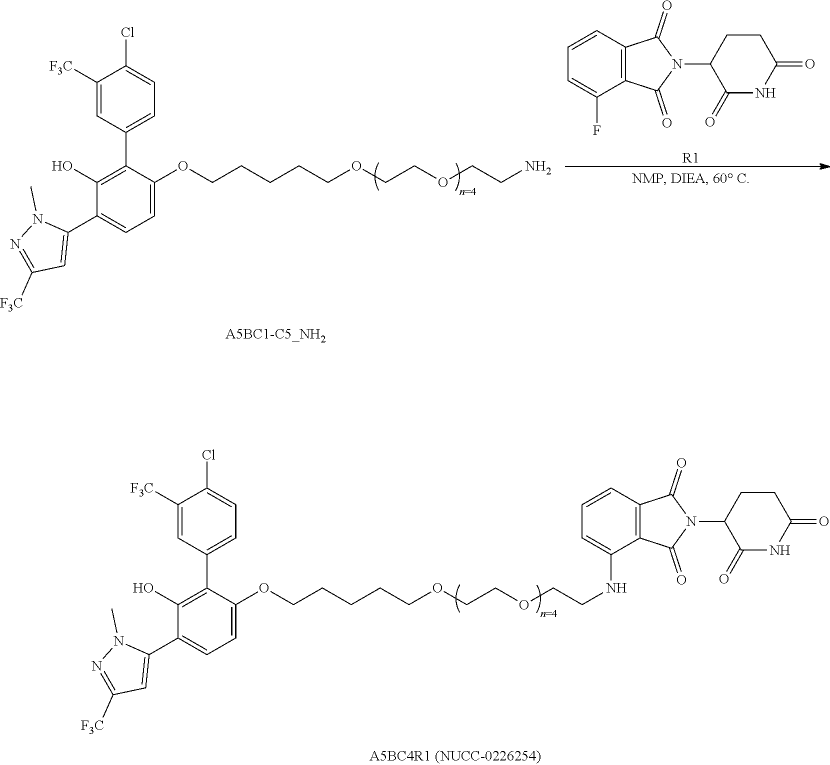

To a mixture of A5BC5_NH2 (150 mg, 190.79 μmol, 1 eq) in NMP (1 mL) was added DIEA (73.97 mg, 572.38 μmol, 99.70 μL, 3 eq) and R1 (57.97 mg, 209.87 μmol, 1.1 eq) in one portion at 20° C. The mixture was stirred at 20° C. for 30 min, then heated to 60° C. and stirred for 12 hours. LC-MS showed the reaction was completed. The reaction was filtered to give a residue which was purified by Prep-HPLC (column: Phenomenex Gemini-NX 150*30 mm*5 um; mobile phase: [water (0.1% TFA)-ACN]; B %: 45%-75%, 9 min) to give A5BC5R1 (40 mg, 20% yield) as white solid.

¹H NMR (ET37412-28-P1B, 400 MHz, CHLOROFORM-d) δ 1.35 (br s, 2H), 1.54 (br d, J=5.62 Hz, 2H), 1.68 (br s, 2H), 2.00-2.46 (m, 2H), 2.66-2.93 (m, 4H), 3.36-3.50 (m, 4H), 3.57 (br s, 2H), 3.64 (br s, 18H), 3.71 (br s, 2H), 3.84 (s, 3H), 3.98 (br s, 2H), 4.89 (br d, J=7.09 Hz, 1H), 6.57 (s, 1H), 6.67 (br d, J=8.44 Hz, 1H), 6.92 (br d, J=8.68 Hz, 1H), 7.10 (br d, J=6.72 Hz, 1H), 7.20 (br d, J=8.44 Hz, 1H), 7.46-7.56 (m, 2H), 7.62 (br d, J=8.07 Hz, 1H), 7.77 (br s, 1H), 8.28 (br s, 1H)

$$LCMS ( ESI + ) : RT = 3.051 min , m / z 1042.1 {( M + H )}^{+} .$$

### 5_95AB_6min_MS1500-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Luna C18 50*2.0 mm column (Sum particles). Detection methods are diode array (DAD) and evaporative light scattering (ELSD) detection. MS mode was positive electrospray ionization.

### Synthesis of A5BC1R³ (NUCC-0226266), A5BC2R3 (NUCC-0226265), A5BC1R5 (NUCC-0226264), A5BC2R5 (NUCC-0226220), A5BC3R5 (NUCC-0226221), A5BC5NH2 (NUCC-0226262), A5BC4R4 (NUCC-0226256), A5BC5R4 (NUCC-0226257)

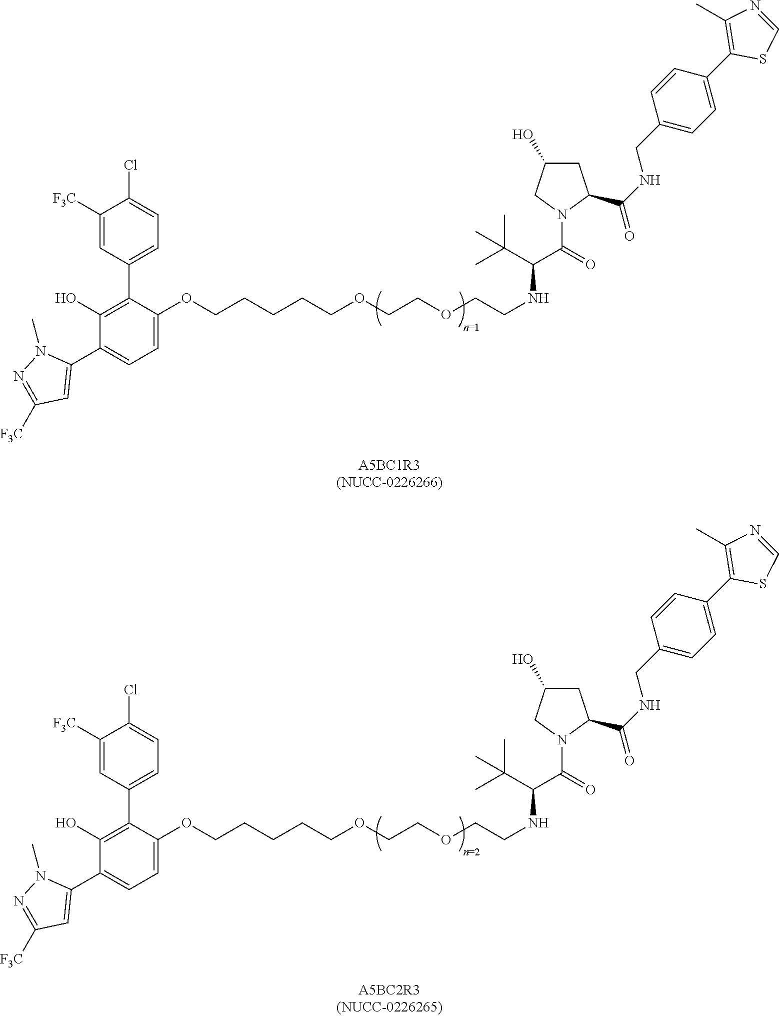

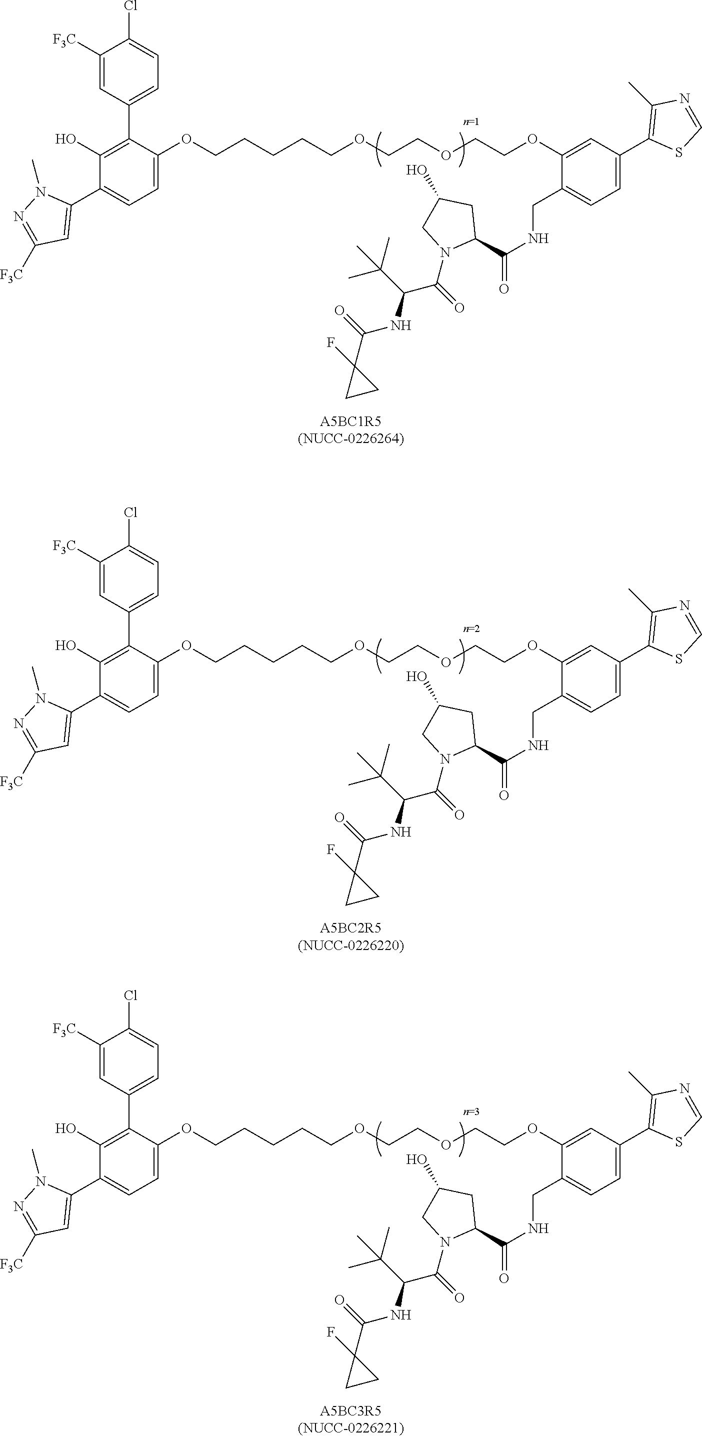

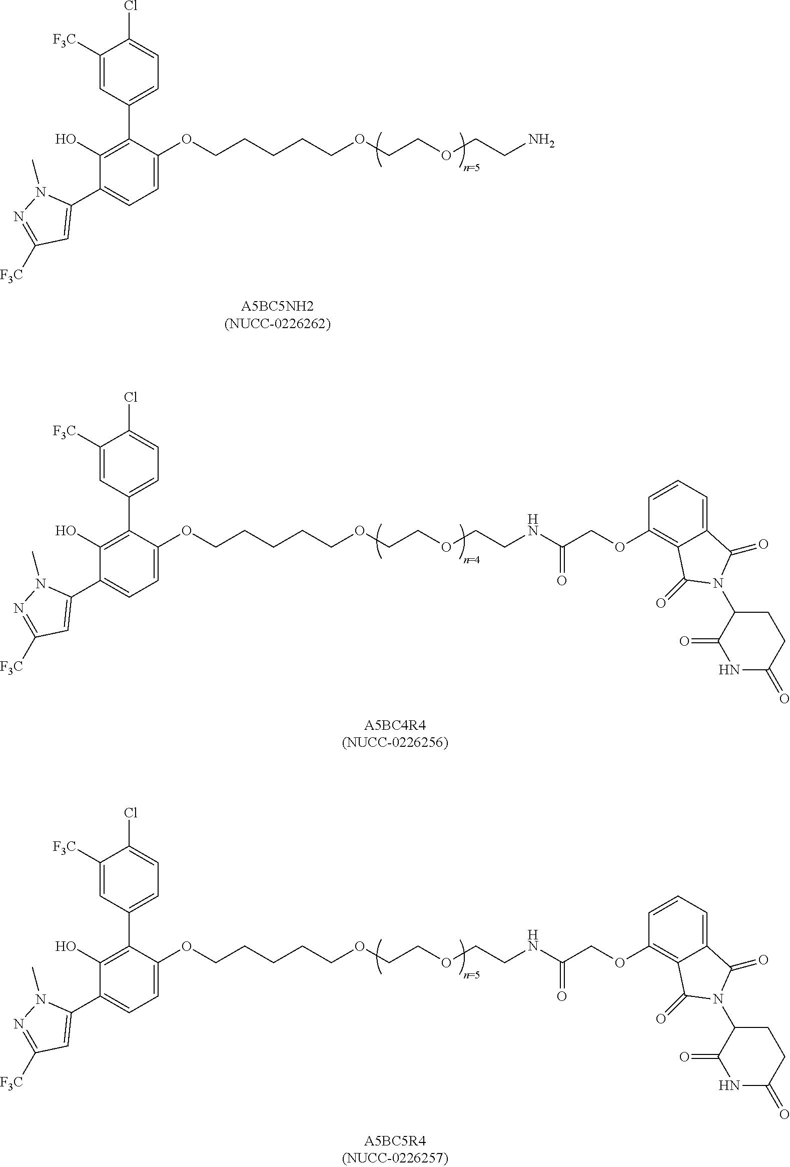

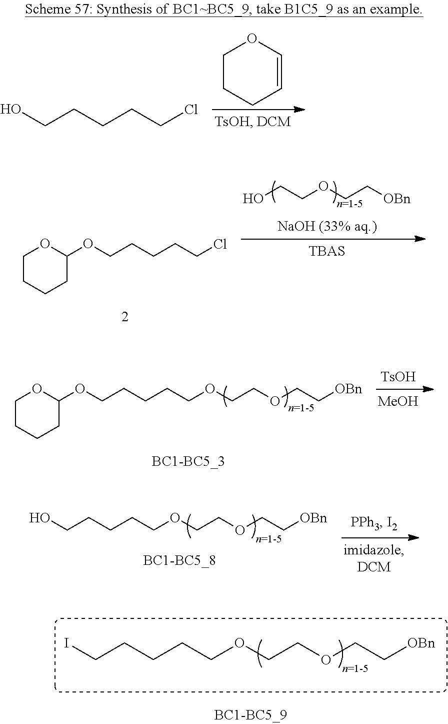

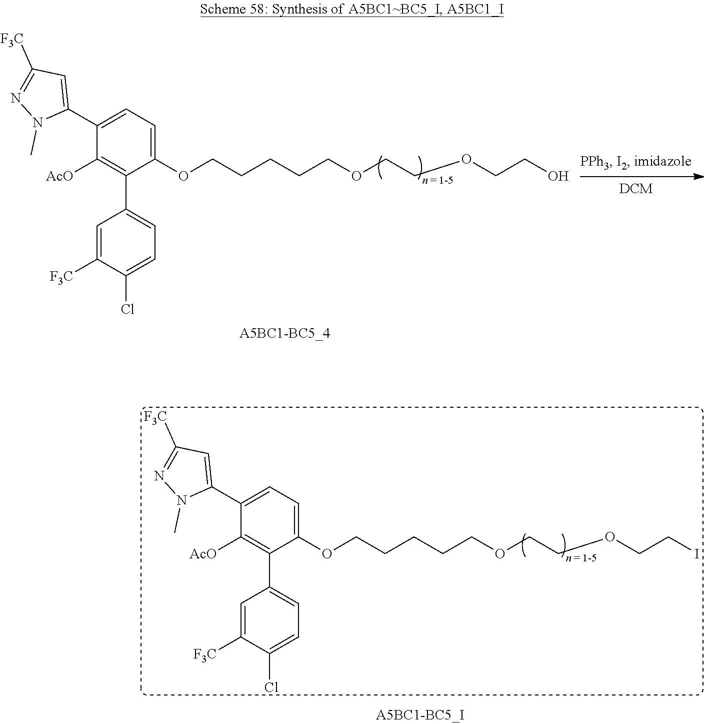

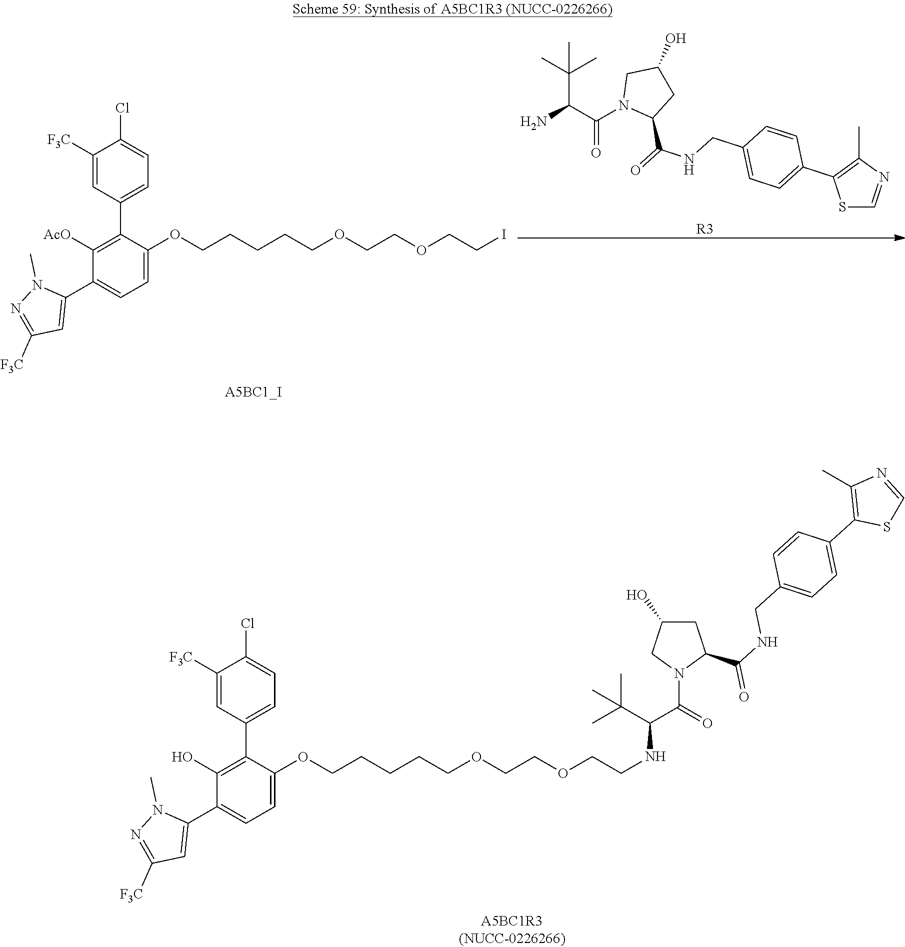

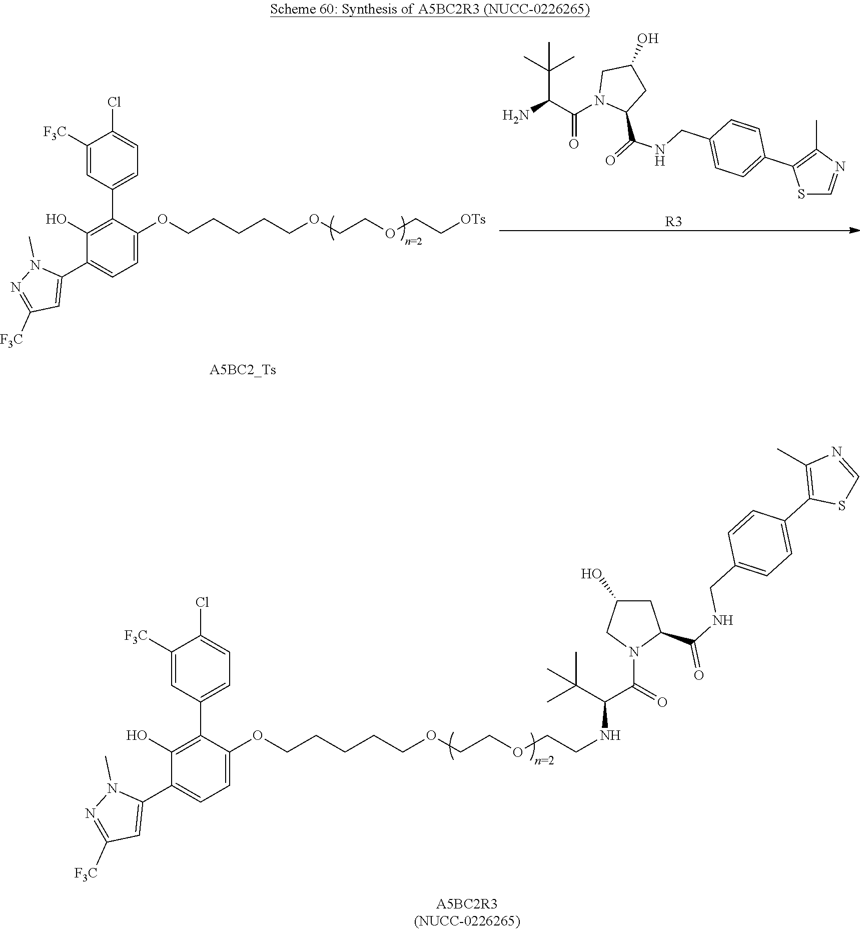

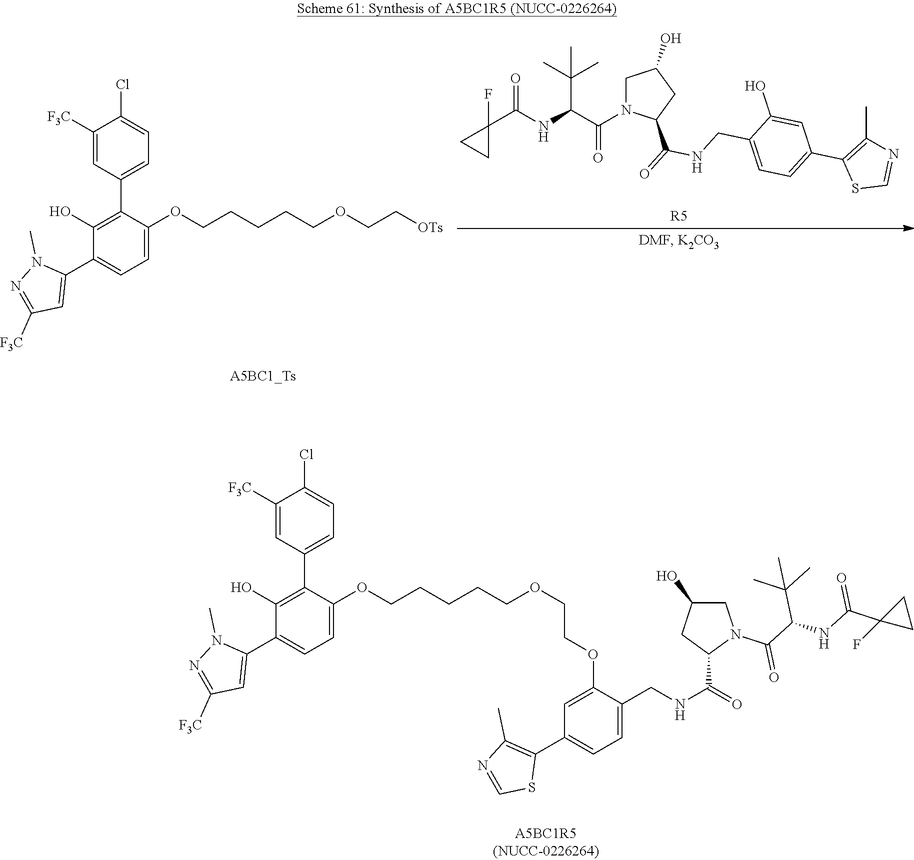

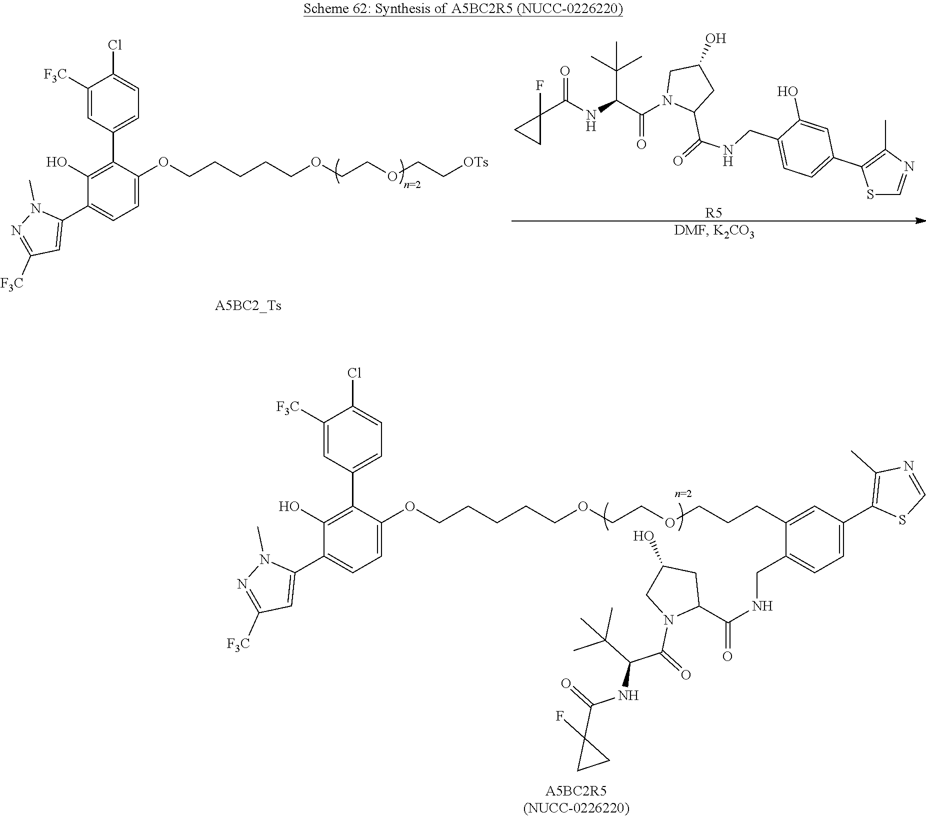

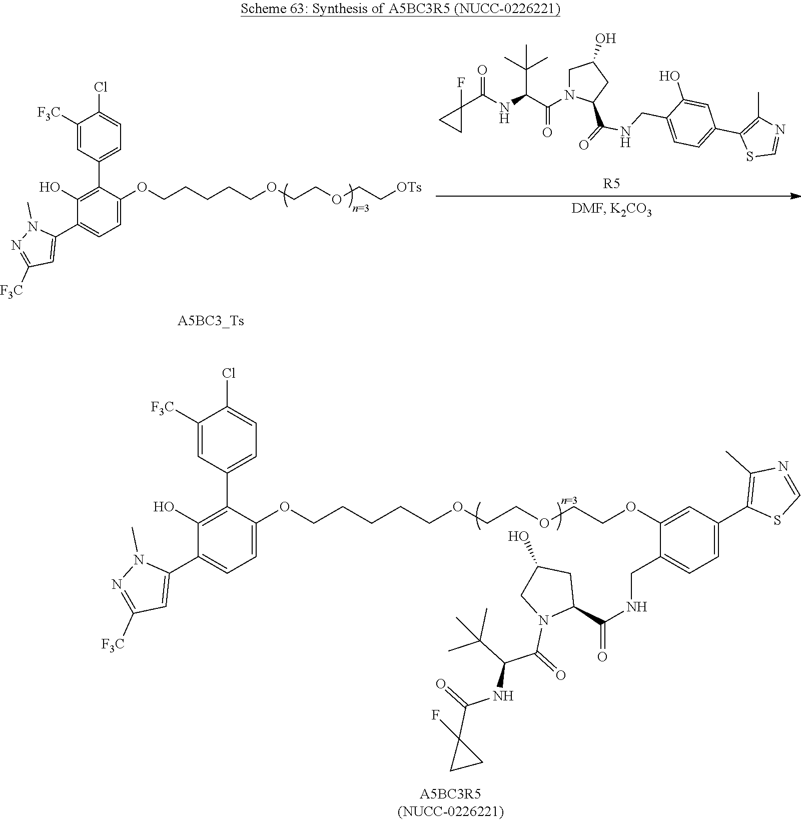

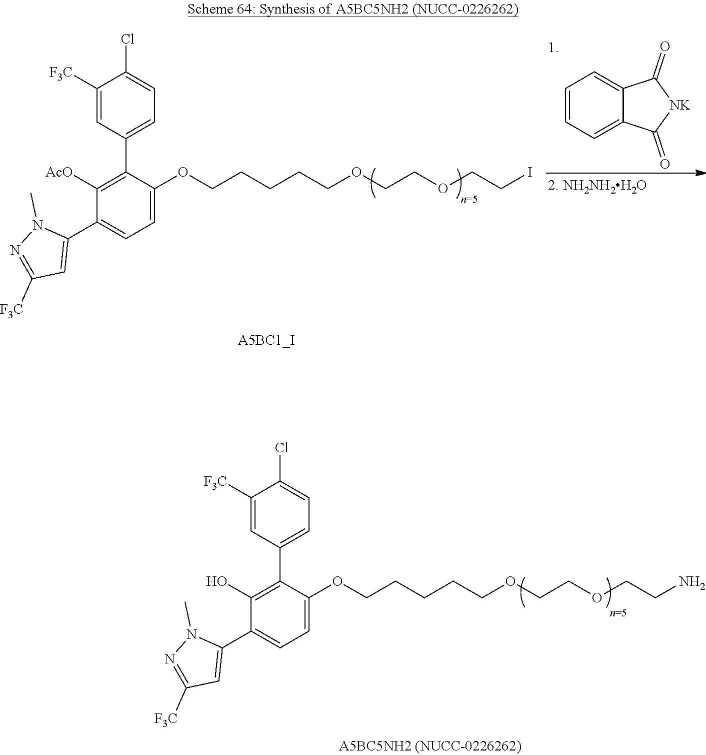

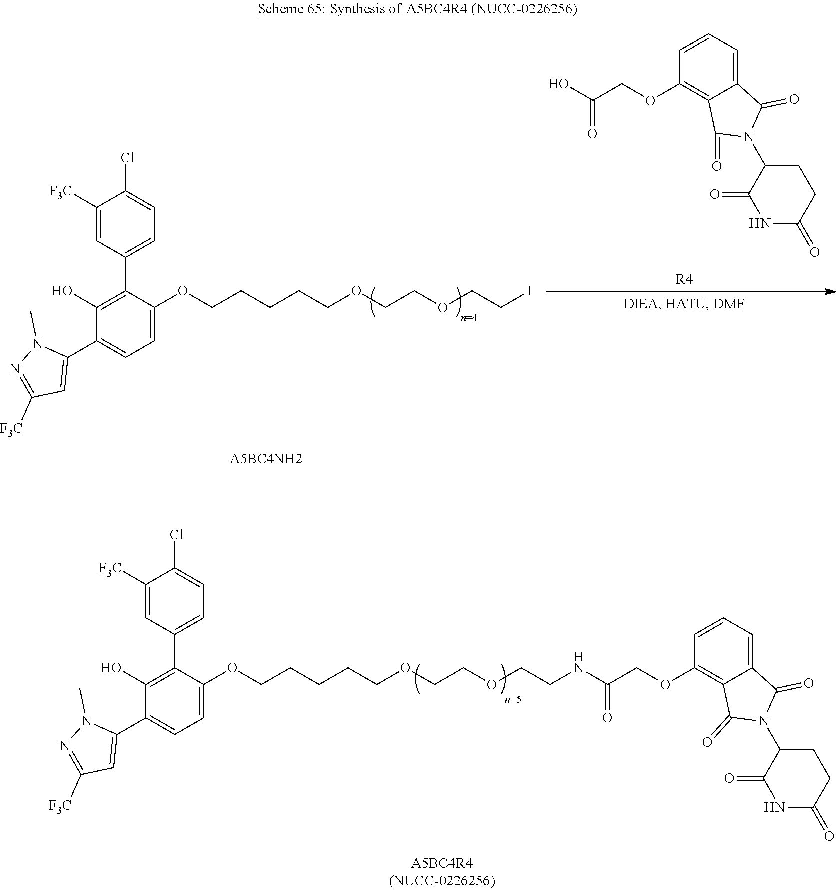

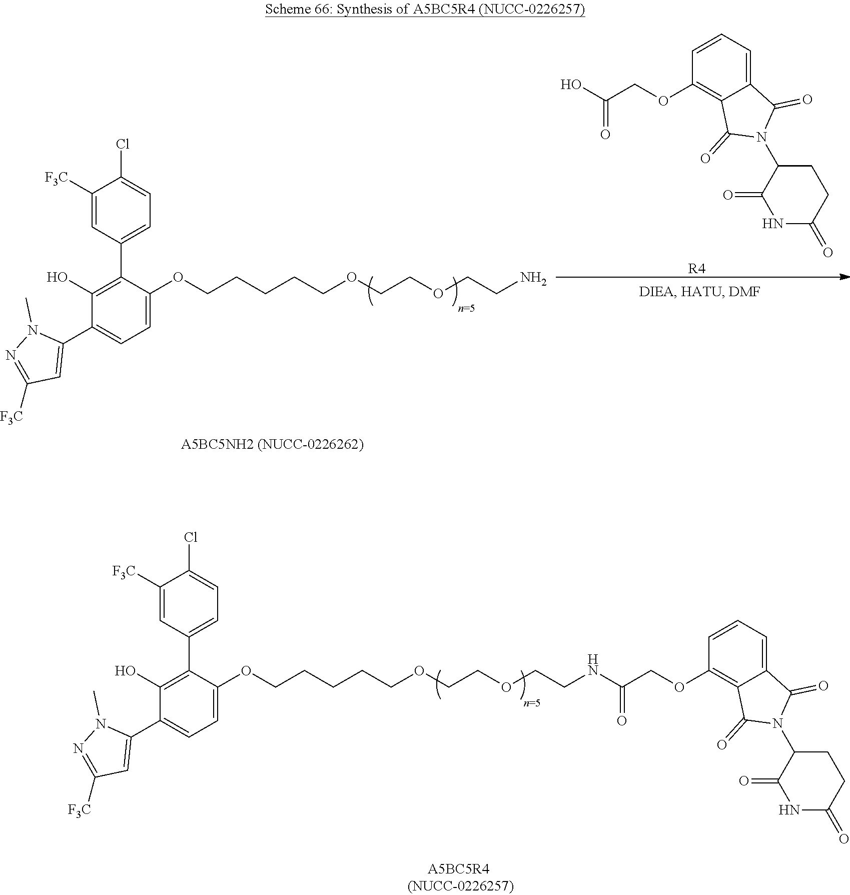

### Chemical Synthesis

### LCMS Methods:

### Method 1:5-95AB_2_min:

LC/MS (The column used for chromatography was a Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

### Method 2: 5_95AB_6min-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. Experimental for largest scale run:

### General Procedure for Preparation of Compound 2-ET32240-983

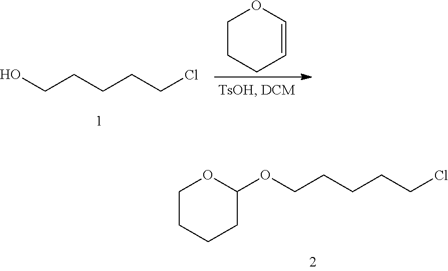

To a solution of Cpd 1 in DCM (500 mL) was added TsOH (7.02 g, 40.79 mmol, 0.1 eq) and then added 3,4-dihydro-2H-pyran (68.61 g, 815.71 mmol, 74.58 mL, 2 eq) dropwise. The mixture was stirred at 25° C. for 4 hrs. TLC showed all starting materials consumed and desired product. The reaction was quenched with K₂CO₃ (sat. 100 mL). The organic layer was concentrated to give crude product which was purified by chromatography on silica, eluted with PE:EA=10:1 to give Cpd 2 (60 g, 290.26 mmol, 71.17% yield) as colorless oil.

¹H NMR (400 MHz, CHLOROFORM-d) δ 1.47-1.90 (m, 13H), 3.32-3.46 (m, 1H), 3.47-3.61 (m, 3H), 3.71-3.81 (m, 1H), 3.87 (ddd, J=11.09, 7.45, 3.28 Hz, 1H), 4.49-4.65 (m, 1H)

### General Procedure for Preparation of Compound BC1_3-ET32240-1163

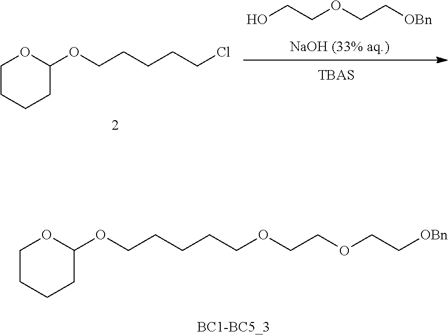 [SMILES: OCCOCCOCc1ccccc1 ; ClCCCCCOC1CCCCO1 ; c1ccc(COCCOCCOCCCCCOC2CCCCO2)cc1]

Tetrabutylammonium;sulfate (1.41 g, 2.42 mmol, 1.39 mL, 0.1 eq) was added dropwise to a mixture of Cpd 2 (5 g, 24.19 mmol, 1 eq) and NaOH (58.63 g, 483.80 mmol, 33% purity, 20 eq) and 2-(2-benzyloxyethoxy) ethanol (5.22 g, 26.61 mmol, 3.78 mL, 1.1 eg). The two-phase mixture was stirred vigorously and heated at 85° C. for 8 hrs. TLC showed all starting materials consumed and desired product. The reaction was quenched with water (100 mL) and then added ethyl acetate (300 mL). The organic layer was concentrated to give crude product. The crude product was purified by chromatography on silica, eluted with PE:EA=10:1 to give BC1_3 (6 g, 16.37 mmol, 67.68% yield) as colorless oil.

### General Procedure for Preparation of Compound BC1_8-ET32240-1171

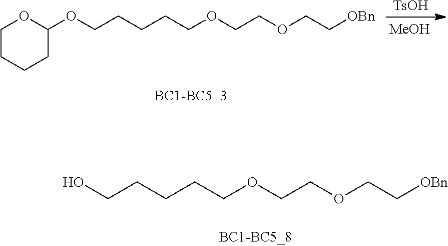 [SMILES: "MeOH" ; c1ccc(COCCOCCOCCCCCOC2CCCCO2)cc1 ; "TsOH" ; OCCCCCOCCOCCOCc1ccccc1]

To a solution of BC1_3 (6 g, 16.37 mmol, 1 eq) in MeOH (100 mL) was added TsOH·H₂O (3.11 g, 16.37 mmol, 1 eq). The mixture was stirred at 25° C. for 2 hrs. TLC showed all starting materials consumed and desired product. The filtrate was concentrated to give a residue and then was added K₂CO₃ (sat. 20 mL) and extracted with EtOAc (3×50 mL). The organic layer was concentrated to give BC1_8 (4 g, 14.17 mmol, 86.53% yield) as colorless oil.

### General Procedure for Preparation of Compound BC2_9-ET32240-1243

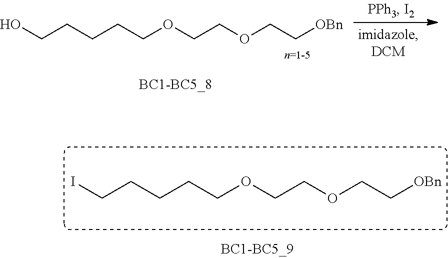 [SMILES: OCCCCCOCCOCCOCc1ccccc1 ; ICCCCCOCCOCCOCc1ccccc1]

A solution of 12 (2.70 g, 10.62 mmol, 2.14 mL, 1.5 eq) and PPh₃ (2.79 g, 10.62 mmol, 1.5 eq) in DCM (40 mL) was added imidazole (964.35 mg, 14.17 mmol, 2 eq). The mixture was stirred for 10 min at 25° C. Then solution of BC1_8 (2 g, 7.08 mmol, 1 eq) in DCM (50 mL) was added dropwise. The mixture was stirred at 25° C. for 8 h. TLC showed all starting material consumed and desired product. The reaction was quenched with water (100 mL) and then added ethyl acetate (300 mL). The organic layer was concentrated to give crude product. The crude product was purified by chromatography on silica, eluted with PE:EA=10:1 to give BC1_9 (1.4 g, 3.57 mmol, 50.39% yield) as colorless oil.

¹H NMR (400 MHz, CHLOROFORM-d) δ 1.43-1.54 (m, 2H), 1.58-1.69 (m, 2H), 1.80-1.95 (m, 2H), 3.21 (t, J=7.03 Hz, 2H), 3.47-3.52 (m, 2H), 3.59-3.65 (m, 2H), 3.65-3.77 (m, 6H), 4.55-4.66 (m, 2H), 7.29-7.41 (m, 5H)

### General Procedure for Preparation of Compound A5BC1_3-ET32240-1244

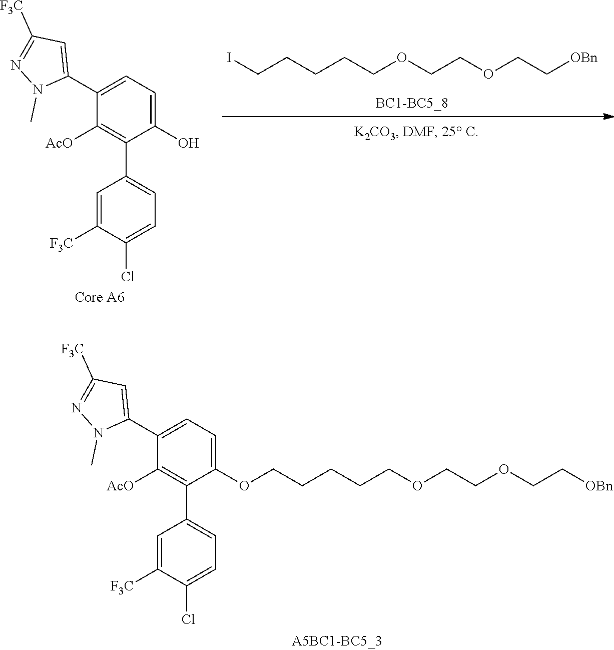 [SMILES: CC(=O)Oc1c(-c2cc(C(F)(F)F)nn2C)ccc(O)c1-c1ccc(Cl)c(C(F)(F)F)c1 ; ICCCCCOCCOCCOCc1ccccc1 ; CC(=O)Oc1c(-c2cc(C(F)(F)F)nn2C)ccc(OCCCCCOCCOCCOCc2ccccc2)c1-c1ccc(Cl)c(C(F)(F)F)c1]

A solution of Core A6 (1.5 g, 3.13 mmol, 1 eq) and BC1_8 (1.35 g, 3.45 mmol, 1.1 eq) in DMF (15 mL) was added K₂CO₃ (519.62 mg, 3.76 mmol, 1.2 eq). The reaction was stirred at 40° C. for 8 hr. TLC and LCMS showed all starting materials consumed and desired product. The reaction was quenched with water (10 mL) and extracted with ethyl acetate (3×20 mL). The organic layer was concentrated to give crude product which was purified by column chromatography on silica (SiO₂, petroleum ether/ethyl acetate=100/1 to 10/1) to give A5BC1_3 (1.5 g, 2.02 mmol, 64.43% yield) as colorless oil.

¹H NMR (400 MHz, CHLOROFORM-d) δ 0.79-1.20 (m, 1H), 1.31-1.44 (m, 3H), 1.58-1.72 (m, 3H), 1.74-1.78 (m, 3H), 3.39-3.44 (m, 1H), 3.44-3.45 (m, 1H), 3.63 (br s, 9H), 3.78-3.82 (m, 3H), 3.99 (t, J=6.28 Hz, 2H), 4.56-4.57 (m, 2H), 6.44-6.50 (m, 1H), 6.93-7.00 (m, 1H), 7.28-7.36 (m, 8H), 7.41-7.46 (m, 1H), 7.51-7.56 (m, 1H), 7.65-7.71 (m, 1H)

$$LCMS ( ESI + ) : m / z = 743.2 ( M + H ) + , RT : 1.693 min .$$

LC/MS (The gradient was 5-95% B in 0.7 min, 95-95% B in 0.45 min, 95-5% B in 0.01 min, and then held at 0% B for 0.44 min (1.5 mL/min flow rate). Mobile phase A was 0.0375% CF₃CO₂H in water, mobile phase B was 0.018% CF₃CO₂H in CH₃CN. The column used for the chromatography is a Chromolith Flash RP-18e 25-2 mm column. Detection methods are diode array (DAD) and evaporative light scattering (ELSD) detection as well as positive electrospray ionization (MS).

### General Procedure for Preparation of Compound A5BC1_4-ET32240-1246

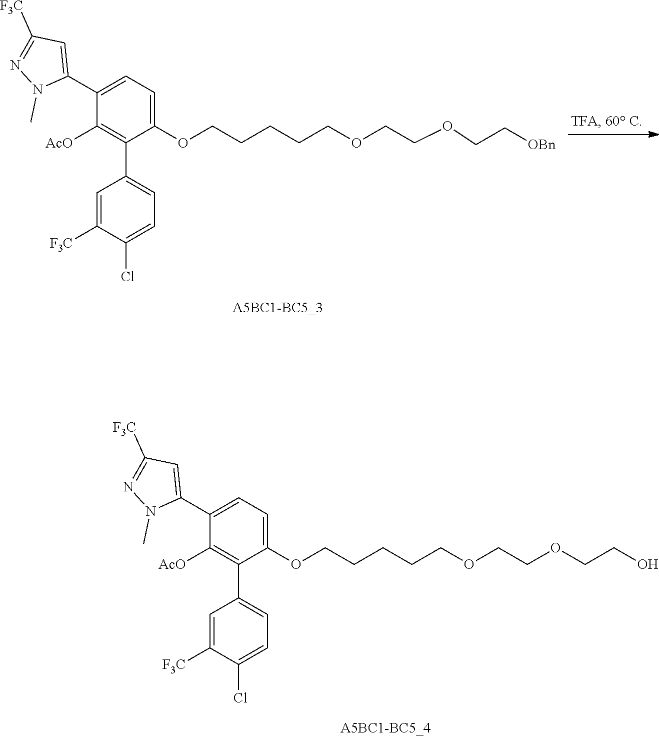 [SMILES: CC(=O)Oc1c(-c2cc(C(F)(F)F)nn2C)ccc(OCCCCCOCCOCCOCc2ccccc2)c1-c1ccc(Cl)c(C(F)(F)F)c1 ; CC(=O)Oc1c(-c2cc(C(F)(F)F)nn2C)ccc(OCCCCCOCCOCCO)c1-c1ccc(Cl)c(C(F)(F)F)c1]

To a solution of A5BC1_3 (1.5 g, 2.02 mmol, 1 eq) in TFA (23.02 g, 201.85 mmol, 14.95 mL, 100 eq) was stirred at 60° C. for 8 hrs. LCMS showed all starting materials consumed. The solution was concentrated to give crude product and then added MeOH (10 mL) and water (1 mL) and then added NaHCO₃ to pH-7. The mixture was stirred for 2 hrs and LCMS showed the desired product. The solution was added EtOAc (50 mL) and water (10 mL). The organic layer was separated and dried over Na₂SO₄, concentrated to give crude product. The crude product was purified by chromatography on silica, eluted with PE:EtOAc=10:1 to EtOAc to give A5BC1_4 (0.5 g, 765.69 μmol, 37.93% yield) as colorless oil.

1H NMR (400 MHz, CHLOROFORM-d) δ 1.30-1.47 (m, 1H), 1.35-1.45 (m, 1H), 1.52-1.59 (m, 4H), 1.69 (br dd, J=14.44, 6.95 Hz, 4H), 1.75-1.80 (m, 3H), 3.38-3.45 (m, 2H), 3.56-3.59 (m, 2H), 3.60-3.63 (m, 2H), 3.65-3.69 (m, 2H), 3.70-3.75 (m, 2H), 3.77-3.86 (m, 3H), 3.98-4.04 (m, 2H), 6.46-6.52 (s, 1H), 6.95-7.01 (d, 1H), 7.30-7.36 (d, 1H), 7.41-7.46 (m, 1H), 7.51-7.56 (m, 1H), 7.67-7.71 (m, 1H)

$$LCMS ( ESI + ) : m / z = 653.1 ( M + H ) + , RT : 0.924 min .$$

LC/MS (The gradient was 5-95% B in 0.7 min. 95-95% B in 0.45 min, 95-5% B in 0.01 min, and then held at 0% B for 0.44 min (1.5 mL/min flow rate). Mobile phase A was 0.0375% CF₃CO₂H in water, mobile phase B was 0.018% CF₃CO₂H in CH₃CN. The column used for the chromatography is a Chromolith Flash RP-18e 25-2 mm column. Detection methods are diode array (DAD) and evaporative light scattering (ELSD) detection as well as positive electrospray ionization (MS).

### General Procedure for Preparation of Compound A5BC1_Ts-ET32240-1250

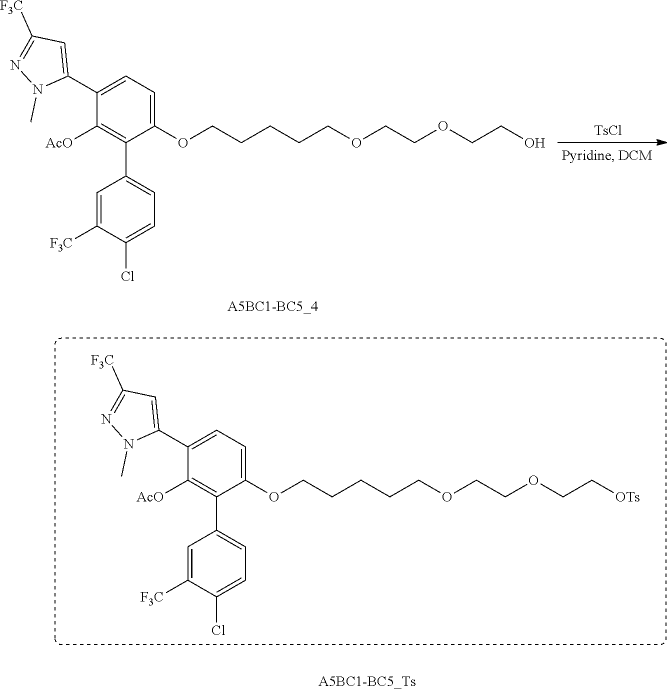 [SMILES: CC(=O)Oc1c(-c2cc(C(F)(F)F)nn2C)ccc(OCCCCCOCCOCCO)c1-c1ccc(Cl)c(C(F)(F)F)c1 ; "TsCl" ; CC(=O)Oc1c(-c2cc(C(F)(F)F)nn2C)ccc(OCCCCCOCCOCCOS(=O)(=O)c2ccc(C)cc2)c1-c1ccc(Cl)c(C(F)(F)F)c1]

To a solution of A5BC1_4 (0.2 g, 306.27 μmol, 1 eq) in DCM (5 mL) was added TsCl (583.90 mg, 3.06 mmol, 10 eq) and pyridine (242.26 mg, 3.06 mmol, 247.21 μL, 10 eq). The mixture was stirred at 25° C. for 8 hrs. LCMS showed almost of starting materials consumed and desired product. The reaction was quenched with water (5 mL) and then added EtOAc (10 mL). The organic layer was concentrated to give crude product. The crude product was purified by Prep-TLC, PE:EA=3:1 to give A5BC1_Ts (0.2 g, 247.77 μmol, 80.90% yield) as colorless oil.

$$LCMS ( ESI + ) : m / z = 807.2 ( M + H ) + , RT : 1.361 min .$$

LC/MS (The gradient was 5-95% B in 0.7 min, 95-95% B in 0.45 min, 95-5% B in 0.01 min, and then held at 0% B for 0.44 min (1.5 mL/min flow rate). Mobile phase A was 0.0375% CF3CO2H in water, mobile phase B was 0.018% CF3CO2H in CH3CN. The column used for the chromatography is a Chromolith Flash RP-18e 25-2 mm column. Detection methods are diode array (DAD) and evaporative light scattering (ELSD) detection as well as positive electrospray ionization (MS).

### General Procedure for Preparation of Compound A5BC1_I-ET32240-1271

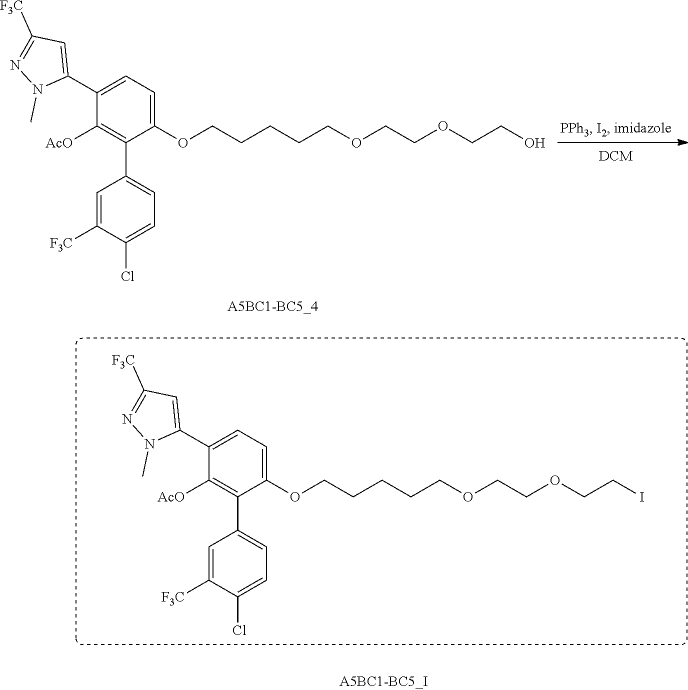

To a solution of A5BC1_4 (0.1 g, 153.14 μmol, 1 eq) in DCM (1 mL) was added PPh3 (60.25 mg, 229.71 μmol, 1.5 eq), imidazole (20.85 mg, 306.27 μmol, 2 eq) and 12 (58.30 mg, 229.71 μmol, 46.27 μL, 1.5 eq). The mixture was stirred at 25° C. for 8 hrs. TLC showed all starting materials consumed and desired product. The reaction was quenched with water (10 mL) and then added ethyl acetate (30 mL). The organic layer was concentrated to give crude product. The crude product was purified by Prep-TLC PE:EA=3:1 to give A5BC1_I (0.04 g, 52.43 μmol, 34.24% yield) as colorless oil.

$$LCMS ( ESI + ) : m / z = 763.1 ( M + H ) + , RT : 1.693 min .$$

LC/MS (The gradient was 5-95% B in 0.7 min, 95-95% B in 0.45 min, 95-5% B in 0.01 min, and then held at 0% B for 0.44 min (1.5 mL/min flow rate). Mobile phase A was 0.0375% CF3CO2H in water, mobile phase B was 0.018% CF3CO2H in CH3CN. The column used for the chromatography is a Chromolith Flash RP-18e 25-2 mm column. Detection methods are diode array (DAD) and evaporative light scattering (ELSD) detection as well as positive electrospray ionization (MS).

### General Procedure for Preparation of Compound A5BC1R3 (NUCC-0226266)-ET32240-1275

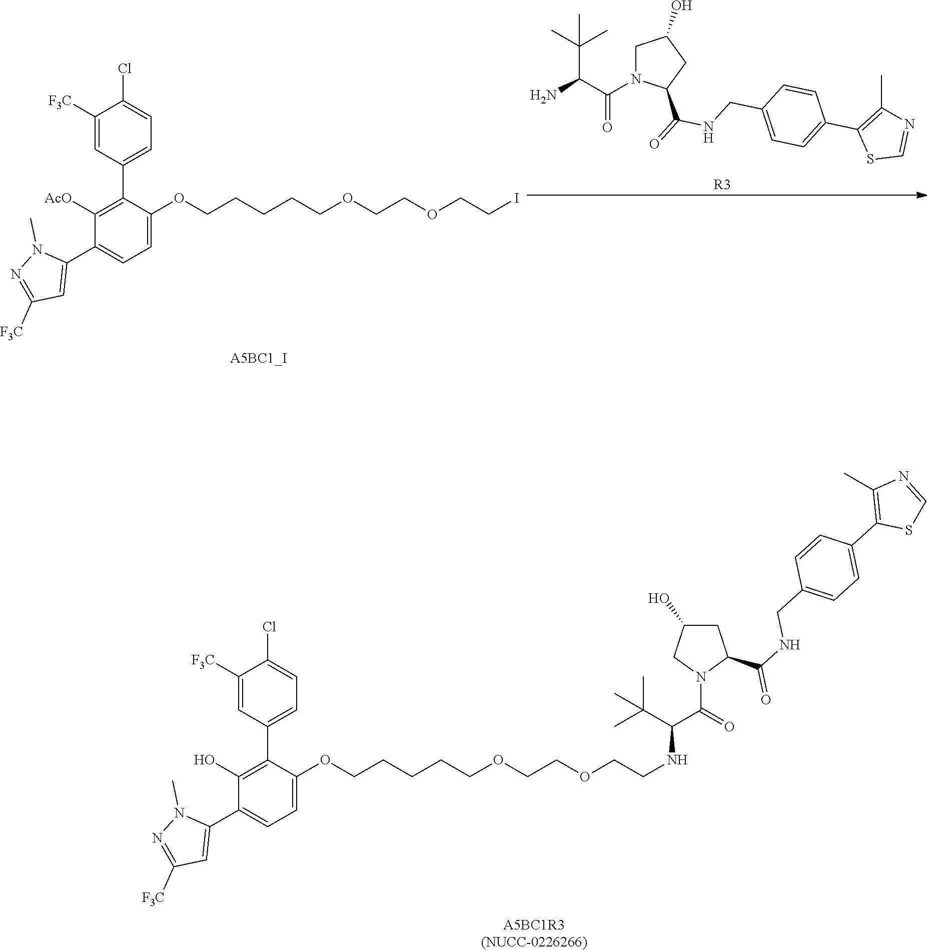

To a mixture of A5BC1_I (30 mg, 39.32 μmol, 1 eq) and R³ (16.38 mg, 39.32 μmol, 1 eq) in DMSO (1 mL) was added DIEA (10.16 mg, 78.64 μmol, 13.70 μL, 2 eq) at 20° C. Then the reaction mixture was stirred at 60° C. for 12 h. Then the reaction mixture were MeOH (1 mL), H₂O (1 mL) and K₂CO₃ (10.87 mg, 78.64 μmol, 2 eq) at 20° C. Then the reaction mixture was stirred at 20° C. for 2 h. LCMS showed all starting materials consumed and desired product. The reaction mixture was quenched with water (10 mL) and extracted with 3×10 mL. The organic layer was concentrated to give crude product. The residue was purified by Prep-HPLC (TFA, column: Phenomenex Luna C18 150*30 mm*5 um; mobile phase: [water (0.1% TFA)-ACN]; B %: 50%-80%, 8 min) to give A5BC1R3 (5.3 mg, 5.13 μmol, 13.04% yield, 99% purity) as white solid.

¹H NMR (400 MHz, CHLOROFORM-d) δ 0.61-0.99 (m, 1H), 1.08 (s, 9H), 1.35 (br d, J=6.85 Hz, 2H), 1.55 (br d, J=7.34 Hz, 2H), 1.63-1.72 (m, 2H), 2.54 (s, 5H), 3.03-3.19 (m, 2H), 3.43 (br t, J=6.85 Hz, 3H), 3.50-3.73 (m, 7H), 3.85 (s, 3H), 3.93-4.01 (m, 4H), 4.37 (br s, 2H), 4.47-4.56 (m, 2H), 4.77-4.85 (m, 1H), 6.57 (s, 1H), 6.67 (d, J=8.80 Hz, 1H), 7.21 (d, J=8.80 Hz, 1H), 7.36 (s, 4H), 7.48-7.57 (m, 2H), 7.58-7.63 (m, 1H), 7.78 (d, J=1.47 Hz, 1H), 8.96 (s, 1H)

### LCMS Method:

$$LCMS ( ESI + ) : m / z = 1023.5 ( M + H ) + , RT : 2.448 min .$$

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (Sum particles). Detection methods are diode array (DAD) and evaporative light scattering (ELSD) detection as well as positive electrospray ionization.

$$LCMS ( ESI + ) : m / z = 1023.1 ( M + H ) + , RT : 2.647 min .$$

LC/MS The gradient: 5% B in 0.01 min, 5-95% B (0.01-1.60 min), 95-100% B (1.60-2.50 min), 100-5% (2.50-2.52 min) with a hold at 5% B for 0.48 min. The flow rate was 0.8 mL/min. Mobile phase A was 0.037% Trifluoroacetic Acid in water, mobile phase B was 0.018% Trifluoroacetic Acid in acetonitrile. The column used for chromatography was a Kromasil Eternity-C18 3.0*30 mm, 2.5 um column (2.5 um particles). Detection methods are diode array (DAD) and evaporative light scattering (ELSD) detection as well as positive electrospray ionization.

### General Procedure for Preparation of Compound A5BC2R³ (NUCC-0226265)-ET32240-1268

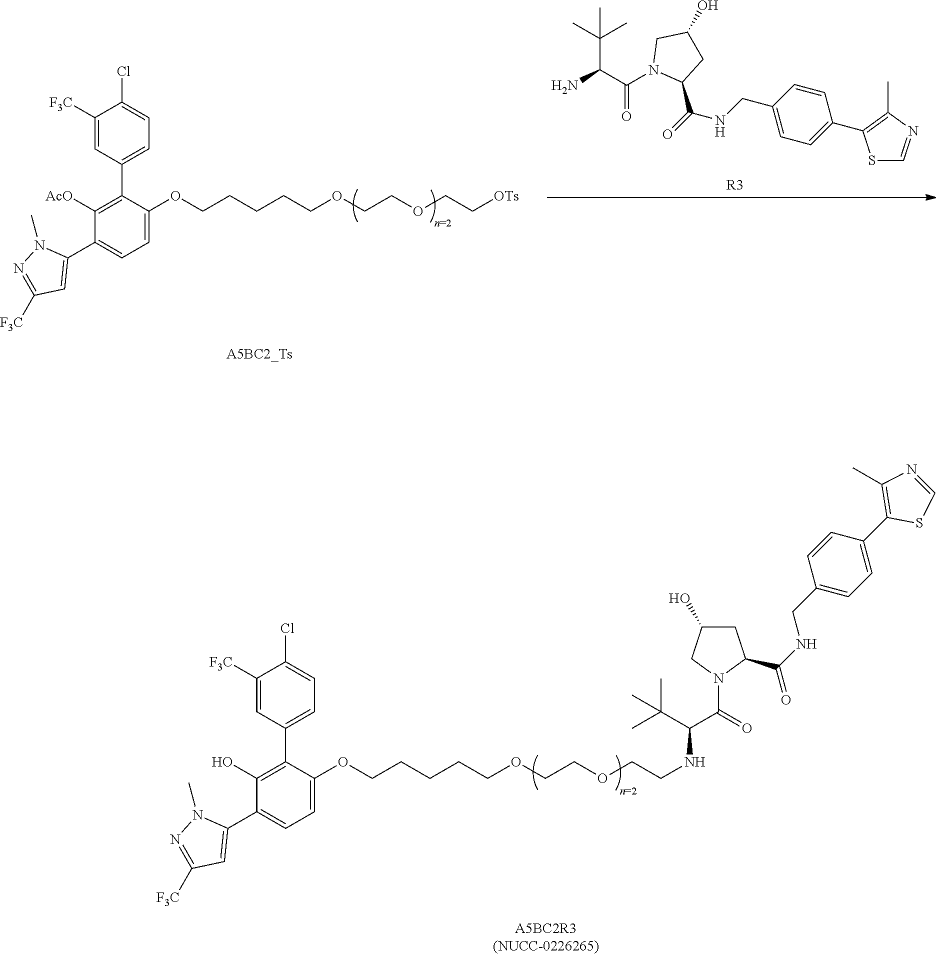

To a solution of R³ (27.82 mg, 64.61 μmol, 1.1 eq) in DMSO (1 mL) was added KI (9.75 mg, 58.74 μmol, 1 eq) and A5BC2_Ts (0.05 g, 58.74 μmol, 1 eq). The mixture was stirred at 50° C. for 8 hrs. LCMS showed almost of starting materials consumed and desired product. Then K₂CO₃ (12.18 mg, 88.11 μmol, 1.5 eq) was added to the mixture and stirred for another 2 hrs at 50° C. The reaction was purified by Prep-HPLC directly (column: Phenomenex luna C18 100*40 mm*5 um; mobile phase: [water (0.1% TFA)-ACN]; B %: 40%-85%, 8 min) to give A5BC2R³ (4.0 mg, 3.75 μmol, 6.38% yield) as white solid.

¹H NMR (400 MHz, CHLOROFORM-d) δ 0.97-1.17 (m, 9H), 1.27-1.38 (m, 2H), 1.47-1.58 (m, 2H), 1.61-1.71 (m, 2H), 2.32 (br dd, J=8.05, 3.86 Hz, 1H), 2.49 (s, 3H), 3.05-3.13 (m, 1H), 3.31-3.40 (m, 3H), 3.49-3.73 (m, 11H), 3.79-3.85 (m, 3H), 3.89-4.02 (m, 4H), 4.30-4.41 (m, 2H), 4.43-4.52 (m, 2H), 4.76 (s, 1H), 6.51-6.58 (m, 1H), 6.61-6.70 (m, 1H), 7.16-7.21 (m, 1H), 7.28-7.39 (m, 4H), 7.49-7.54 (m, 1H), 7.55-7.61 (m, 1H), 7.70-7.76 (m, 1H), 7.77-7.85 (m, 1H), 8.76-8.85 (m, 1H)

### LCMS Method:

$$LCMS ( ESI + ) : m / z = 1111.1 ( M + H ? ) + , RT : 1.227 min .$$

LC/MS (The gradient was 5-95% B in 1.0 min, 95-100% B in 0.80 min, 100-5% B in 0.01 min, and then held at 5% B for 0.39 min (1.0 mL/min flow rate). Mobile phase A was 0.0375% CF₃CO₂H in water, mobile phase B was 0.018% CF₃CO₂H in CH₃CN. The column used for the chromatography was a ZORBAX Eclipse XDB-C18 2.1*30 mm, 3.5 um. Detection methods are diode array (DAD) and positive electrospray ionization (MS).

$$LCMS ( ESI + ) : m / z = 534.2 ( M / 2 + H ) + , RT : 2.647 min .$$

LC/MS The gradient: 5% B in 0.01 min, 5-95% B (0.01-1.60 min), 95-100% B (1.60-2.50 min), 100-5% (2.50-2.52 min) with a hold at 5% B for 0.48 min. The flow rate was 0.8 mL/min. Mobile phase A was 0.037% Trifluoroacetic Acid in water, mobile phase B was 0.018% Trifluoroacetic Acid in acetonitrile. The column used for chromatography was a Kromasil Eternity-C18 3.0*30 mm, 2.5 um column (2.5 um particles). Detection methods are diode array (DAD) and evaporative light scattering (ELSD) detection as well as positive electrospray ionization.

### General Procedure for Preparation of Compound A5BC1R⁵ (NUCC-0226264)-ET32240-1253

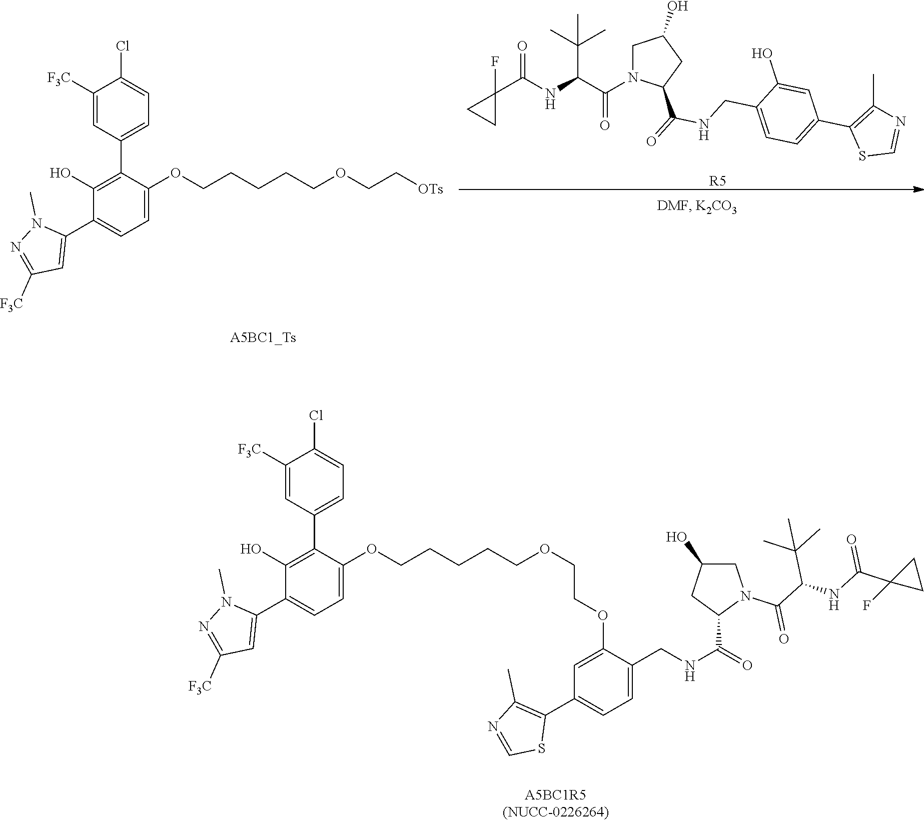

To a solution of A5BC1_I (100 mg, 123.89 μmol, 1 eq) in DMSO (2 mL) was added R5 (131.97 mg, 247.77 μmol, 2 eq), KI (2.06 mg, 12.39 μmol, 0.1 eq) and K₂CO₃ (34.24 mg, 247.77 μmol, 2 eq). The mixture was stirred at 50° C. for 5 h. LCMS showed all starting materials consumed and desired product.

The reaction mixture was quenched with water (10 mL) and extracted with 3×10 mL. The organic layer was concentrated to give crude product. The solution was purified by Prep-HPLC (TFA) column: Phenomenex Luna C18 150*30 mm*5 um; mobile phase: [water (0.1% TFA)-ACN]; B %: 50%-80%, 8 min to give A5BC1R5 (9 mg, 7.76 μmol, 6.26% yield, 97% purity) as white solid.

¹H NMR (400 MHz, CHLOROFORM-d) δ 0.93 (s, 9H), 1.22-1.31 (m, 6H), 1.31-1.40 (m, 4H), 1.54 (br d, J=6.85 Hz, 2H), 2.07-2.14 (m, 1H), 2.34-2.40 (m, 1H), 2.51 (s, 3H), 3.39-3.47 (m, 2H), 3.55-3.64 (m, 3H), 3.71-3.80 (m, 2H), 3.84 (s, 3H), 3.89-4.00 (m, 5H), 4.21 (br dd, J=7.09, 4.65 Hz, 2H), 4.41-4.50 (m, 4H), 4.64 (1, J=8.07 Hz, 1H), 6.55 (s, 1H), 6.65 (d, J=8.80 Hz, 1H), 6.90 (d, J=1.47 Hz, 1H), 6.96 (dd, J=7.83, 1.47 Hz, 1H), 7.01 (s, 1H), 7.20 (d, J=8.31 Hz, 1H), 7.22-7.25 (m, 1H), 7.31 (d, J=7.82 Hz, 1H), 7.52-7.56 (m, 1H), 7.60-7.64 (m, 1H), 7.78 (d, J=1.96 Hz, 1H), 8.70 (s, 1H)

### LCMS Method:

$$LCMS ( ESI + ) : m / z = 1125.6 ( M + H ) + , RT : 3.079 min .$$

LC/MS (The gradient was 25-100% B in 3.4 min with a hold at 100% B for 0.45 min, 100-25% B in 0.01 min, and then held at 25% B for 0.65 min (0.8 mL/min flow rate). Mobile phase A was 0.0375% CF3CO2H in water, mobile phase B was 0.018% CF3CO2H in HPLC grade CH3CN. The column used for the chromatography is a 3.0×50 mm Shim-pack XR-ODS column (5 μm particles). Detection methods are diode array (DAD) and evaporative light scattering (ELSD) detection as well as positive electrospray ionization (MS).

$$LCMS ( ESI + ) : m / z = 563.4 ( M / 2 + H ) + , RT : 2.1 min .$$

LC/MS The gradient: 5% B in 0.01 min, 5-95% B (0.01-1.60 min), 95-100% B (1.60-2.50 min), 100-5% (2.50-2.52 min) with a hold at 5% B for 0.48 min. The flow rate was 0.8 mL/min. Mobile phase A was 0.037% Trifluoroacetic Acid in water, mobile phase B was 0.018% Trifluoroacetic Acid in acetonitrile. The column used for chromatography was a Kromasil Eternity-C18 3.0*30 mm, 2.5 um column (2.5 um particles). Detection methods are diode array (DAD) and evaporative light scattering (ELSD) detection as well as positive electrospray ionization.

### General Procedure for Preparation of Compound A5BC2R⁵ (NUCC-0226220)-ET32240-1247

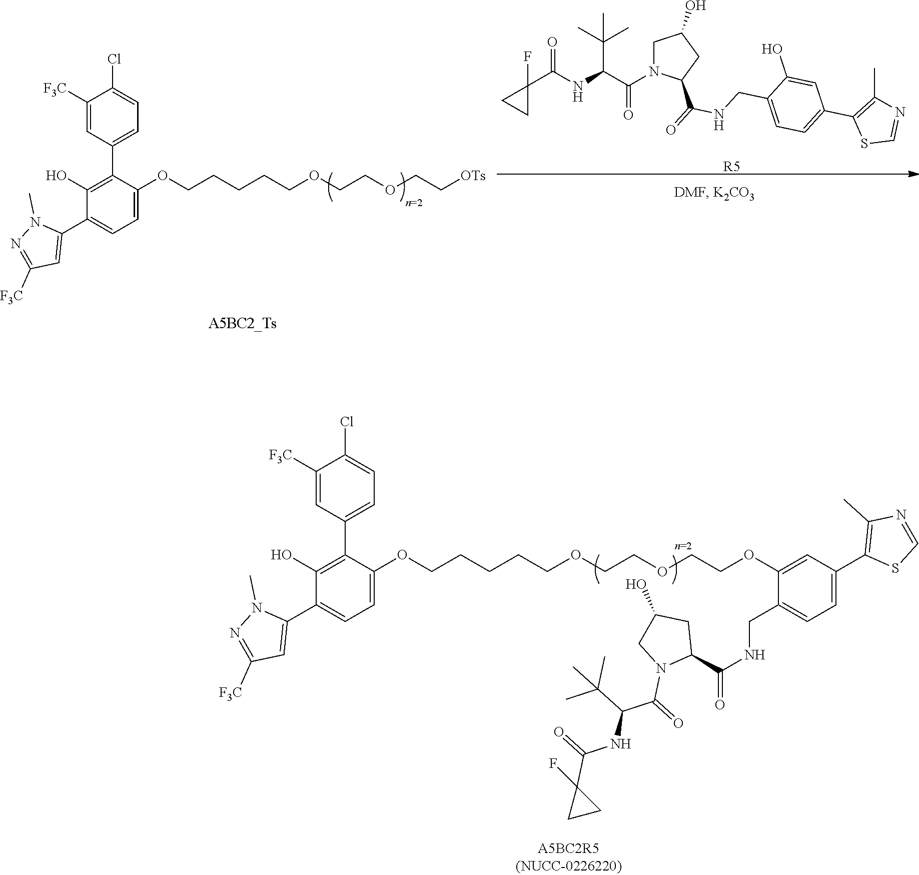

To a solution of A5BC2_Ts (130 mg, 152.72 μmol, 1 eq) in DMSO (2 mL) was added R⁵ (162.68 mg, 305.43 μmol, 2 eq), KI (2.54 mg, 15.27 μmol, 0.1 eq) and K₂CO₃ (42.21 mg, 305.43 μmol, 2 eq). The mixture was stirred at 70° C. for 5 h. LCMS showed all starting materials consumed and desired product. The reaction mixture was quenched with water (1.0 mL) and extracted with EtOAc (3×10 mL). The organic layer was concentrated to give crude product. The crude product was purified by Prep-HPLC (TFA: column: Phenomenex Luna C18 150*30 mm*5 um; mobile phase: [water (0.1% TFA)-ACN]; B %: 50%-80%, 8 min) to give A5BC2R⁵ (9.4 mg, 8.04 μmol, 5.26% yield, 100% purity) as white solid.

¹H NMR (400 MHz, METHANOL-d₄) δ 1.02 (s, 9H), 1.27-1.38 (m, 6H), 1.44-1.53 (m, 2H), 1.59-1.67 (m, 2H), 2.03-2.13 (m, 1H), 2.19-2.28 (m, 1H), 2.49 (s, 3H), 3.35-3.41 (m, 2H), 3.51-3.55 (m, 2H), 3.59-3.63 (m, 2H), 3.64-3.69 (m, 2H), 3.70-3.75 (m, 2H), 3.78 (s, 3H), 3.81-3.87 (m, 1H), 3.90 (br s, 2H), 3.96 (s, 2H), 4.22 (br d, J=3.09 Hz, 2H), 4.37-4.44 (m, 1H), 4.46 (s, 2H), 4.59-4.64 (m, 1H), 4.72-4.76 (m, 1H), 6.57 (s, 1H), 6.74 (s, 1H), 7.04 (s, 2H), 7.21 (d, J=8.60 Hz, 1H), 7.45-7.54 (m, 2H), 7.55-7.59 (m, 1H), 7.63 (s, 1H), 7.72 (s, 1H), 8.93-8.96 (m, 1H)

$$LCMS ( ESI + ) : m / z = 585.2 ( M / 2 + H ) + , RT : 2.079 min .$$

50_100AB_6min-220-254-ELSD: LC/MS (The gradient was 50% B in 0.40 min and 50-100% B at 0.40-3.00 min, hold on 100% B for 1.00 min, and then 100-50% B in 0.01 min, the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization.

### General Procedure for Preparation of Compound A5BC3R⁵ (NUCC-0226221)-ET32240-1252

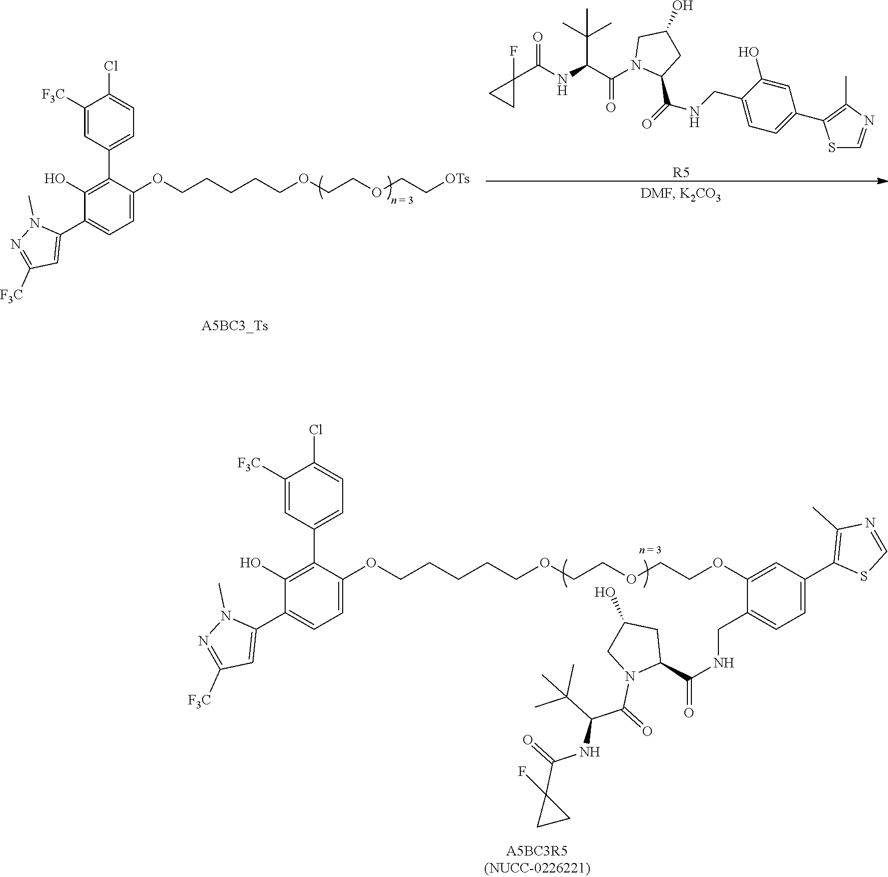

To a solution of A5BC3_Ts (20 mg, 22.34 μmol, 1 eq) in DMSO (2 mL) was added R⁵ (23.80 mg, 44.68 μmol, 2 eq), KI (370.83 ug, 2.23 μmol, 0.1 eq) and K₂CO₃ (6.17 mg, 44.68 μmol, 2 eq). The mixture was stirred at 70° C. for 5 h. LCMS showed all starting materials consumed and desired product. The reaction mixture was quenched with water (10 mL) and extracted with 3×10 mL. The organic layer was concentrated to give crude product. The residue was purified by Prep-HPLC (TFA, column: Phenomenex Luna C18 150*30 mm*5 um; mobile phase: [water (0.1% TFA)-ACN]; B %: 50%-80%, 8 min) to give A5BC3R⁵ (4.2 mg, 3.46 μmol, 15.49% yield, 100% purity) as white solid 1H NMR (400 MHz, METHANOL-d₄) δ 1.02 (s, 9H), 1.29-1.38 (m, 6H), 1.48 (br d, J=7.83 Hz, 2H), 1.64 (br s, 2H), 2.04-2.13 (m, 1H), 2.16-2.28 (m, 1H), 2.49 (s, 3H), 3.35-3.40 (m, 2H), 3.50-3.55 (m, 2H), 3.57-3.64 (m, 7H), 3.65-3.69 (m, 2H), 3.72 (dd, J=4.16, 2.20 Hz, 2H), 3.79 (s, 3H), 3.82 (s, 1H), 3.90 (t, J=4.65 Hz, 2H), 3.97 (t, J=6.11 Hz, 2H), 4.22 (br d, J=3.42 Hz, 2H), 4.42 (s, 1H), 4.46 (s, 2H), 4.58-4.64 (m, 1H), 4.73 (br d, J=8.80 Hz, 1H), 6.55 (s, 1H), 6.72 (d, J=8.31 Hz, 1H), 6.99-7.05 (m, 2H), 7.20 (d, J=8.80 Hz, 1H), 7.46 (br d, J=8.31 Hz, 2H), 7.57 (d, J=1.96 Hz, 1H), 7.60 (s, 1H), 7.73 (d, J=1.47 Hz, 1H), 7.82 (s, 1H), 8.92 (s, 1H)

### LCMS Method:

$$LCMS ( ESI + ) : m / z = 1213.6 ( M + H ) + , RT : 3.314 min .$$

LC/MS (The gradient was 5% B in 0.40 min and 5-95% B at 0.40-3.00 min, hold on 95% B for 1.00 min, and then 95-5% B in 0.01 min, the flow rate was 1.0 ml/min.

Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization.

### General Procedure for Preparation of Compound A5BC5NH₂ (NUCC-0226262)-ET37412-88

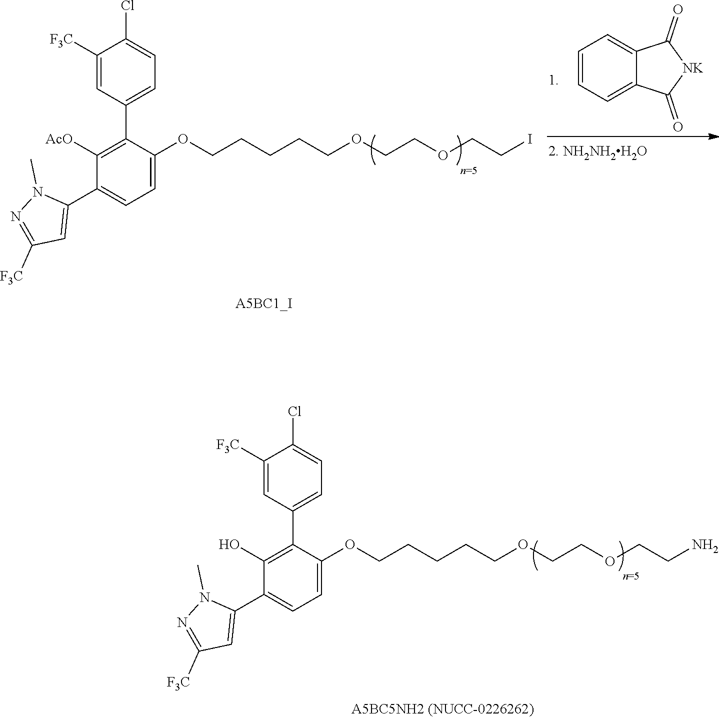

To a solution of A2BC1_1 (50 mg, 53.3 μmol, 1 eq) in DMF (1 mL) was added potassium 1,3-dioxoisoindolin-2-ide (15 mg, 80 μmol, 1 eq) at 20° C., then the reaction mixture was stirred at 60° C. for 4 hrs. Then the above solution was concentrated and the residue was dissolved in EtOH (2 mL) was added NH₂NH₂·H₂O (11 mg, 213 μmol, 4 eq), and the reaction mixture was heated at 60° C. for 2 hrs. LCMS showed most of starting material consumed and desired product was detected. The reaction was cooled to 20° C., filtered, and the filtrate was purified by acidic Prep-HPLC to give A5BC5NH₂ (NUCC-0226262) (8 mg, yield 18.2%) as a white solid.

¹H NMR (400 MHz, CDCl3) δ 1.33-1.36 (m, 2H), 1.53-1.57 (m, 2H), 1.66-1.72 (m, 2H), 3.10-3.12 (m, 2H), 3.40-3.42 (m, 2H), 3.58-3.68 (m, 20H), 3.72-3.74 (m, 2H), 3.84 (s, 3H), 3.99-4.01 (m, 2H), 6.57 (s, 1H), 6.68-6.70 (m, 2H), 7.21-7.28 (m, 2H), 7.54-7.64 (m, 2H), 7.78 (s, 1H), 8.00 (s, 2H)

¹⁹F NMR (400 MHz, CDCl₃) δ −62.002, −62.477, −75.430 LCMS method:

$$LCMS ( ESI + ) : m / z = 786.1 ( M + H ) + , RT : 2.61 min .$$

### 5_95AB_6min-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization.

### General Procedure for Preparation of Compound A5BC4R⁴ (NUCC-0226256)-ET32240-1215

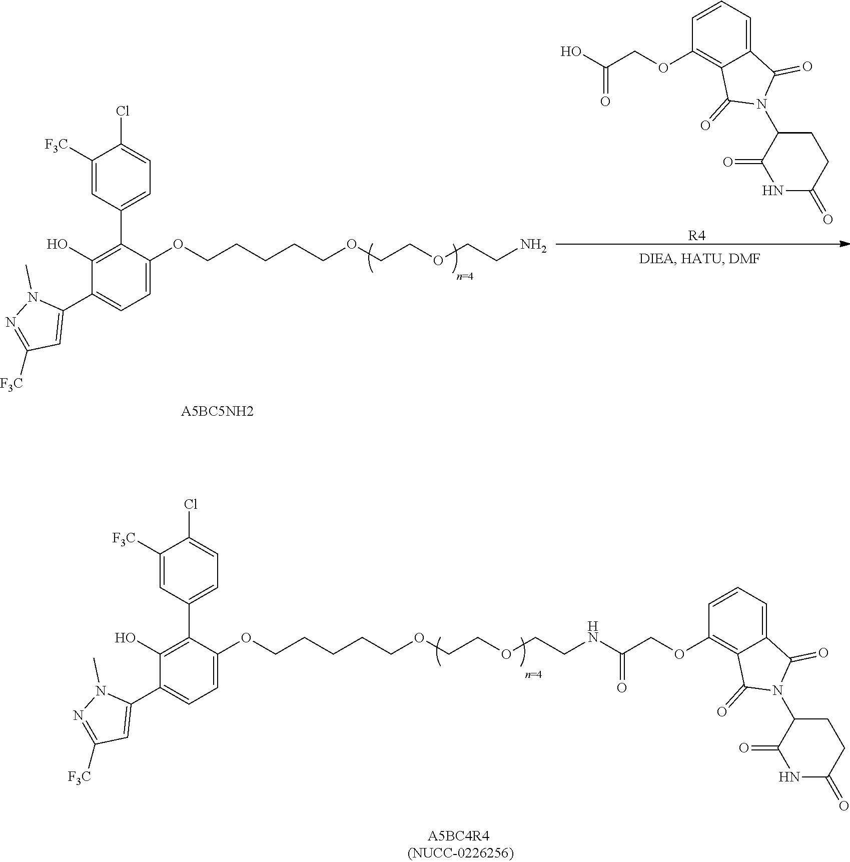

To a solution of R⁴ (24.62 mg, 74.11 μmol, 1.1 eq), A5BC4NH₂ (50 mg, 67.37 μmol, 1 eq), DIEA (26.12 mg, 202.12 μmol, 35.21 μL, 3 eq) and HATU (28.18 mg, 74.11 μmol, 1.1 eq) in DMF (0.5 mL) at 20° C. And the mixture reaction was heated at 80° C. for 4 hours. LCMS showed all starting materials consumed and desired product was generated. The reaction mixture was purified by Prep-HPLC (TFA buffer, Phenomenex Luna C18 150*30 mm*5 um; mobile phase: [water (0.1% TFA)-ACN]; B %: 50%-80%, 8 min) to give A5BC4R⁴ (18 mg, 18.26 μmol, 27.10% yield) as white solid.

¹H NMR (400 MHz, CDCl3) δ 1.23-1.25 (m, 1H), 1.33-1.36 (m, 2H), 1.53-1.57 (m, 2H), 1.66-1.72 (m, 2H), 2.12-2.14 (m, 1H), 2.75-2.87 (m, 4H), 3.39-3.41 (m, 2H), 3.57-3.68 (m, 20H), 3.84 (s, 3H), 3.95-3.98 (m, 2H), 4.67 (s, 1H), 4.91-4.96 (m, 1H), 6.57 (s, 1H), 7.18-7.27 (m, 2H), 7.53-7.55 (m, 2H), 7.59-7.61 (m, 2H), 7.72-7.76 (m, 3H), 8.83 (s, 1H)

¹⁹F NMR (400 MHz, CDCl₃) δ−61.987, −62.605, −75.857

### LCMS Method:

$$LCMS ( ESI + ) : m / z = 1056.1 ( M + H ) + , RT : 2.916 min .$$

### 5_95AB_6min-220-254-ELSD

LC/MS (The gradient was 5% B in 0.40 min and 5-95% B at 0.40-3.00 min. hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization.

### General Procedure for Preparation of Compound A5BC5R⁴ (NUCC-0226257)-ET32240-1216

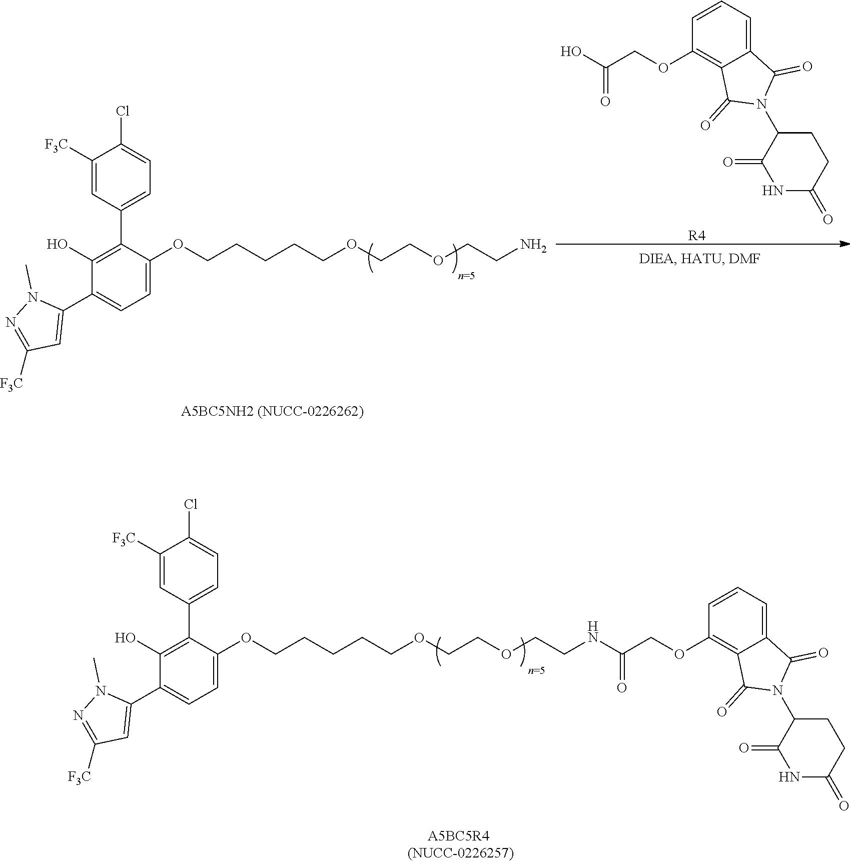

To a solution of A5BC5NH₂ (50 mg, 63.60 μmol, 1 eq), R⁴ (23.24 mg, 69.96 μmol, 1.1 eq), DIEA (24.66 mg, 190.79 μmol, 33.23 μL, 3 eq) and HATU (26.60 mg, 69.96 μmol, 1.1 eq) in DMF (0.5 mL) at 20° C. And the mixture reaction was heated at 80° C. for 4 hours. LCMS showed all starting materials consumed and desired product was generated. The reaction mixture was purified by Prep-HPLC directly (TFA buffer, Phenomenex luna C18 100*40 mm*5 um; mobile phase: [water (0.1% TFA)-ACN]; B %: 40%-72%, 8 min) to give A5BC5R⁴ (36 mg, 32.71 μmol, 51.44% yield) as white solid.

¹H NMR (400 MHz, CDCl₃) δ 1.26-1.27 (m, 1H), 1.33-1.36 (m, 2H), 1.53-1.57 (m, 2H), 1.66-1.72 (m, 2H), 2.12-2.14 (m, 1H), 2.75-2.87 (m, 4H), 3.39-3.41 (m, 2H), 3.55-3.65 (m, 24H), 3.84 (s, 3H), 3.95-3.98 (m, 2H), 4.67 (s, 1H), 4.91-4.96 (m, 1H), 6.57 (s, 1H), 7.18-7.27 (m, 2H), 7.53-7.55 (m, 2H), 7.59-7.61 (m, 2H), 7.72-7.76 (m, 3H), 8.85 (s, 1H)

¹⁹F NMR (400 MHz, CDCl₃) δ −61.991, −62.511, −75.785

### LCMS Method:

$$LCMS ( ESI + ) : m / z = 1100.1 ( M + H ) + , RT : 2.911 min .$$

### 5_95AB_6min-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization

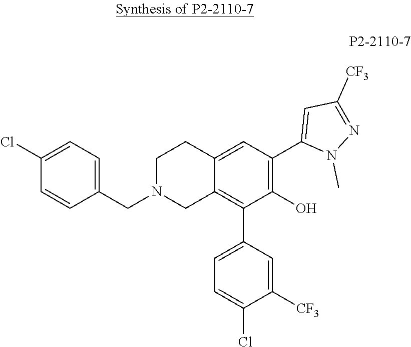 [SMILES: Cn1nc(C(F)(F)F)cc1-c1cc2c(c(-c3ccc(Cl)c(C(F)(F)F)c3)c1O)CN(Cc1ccc(Cl)cc1)CC2]

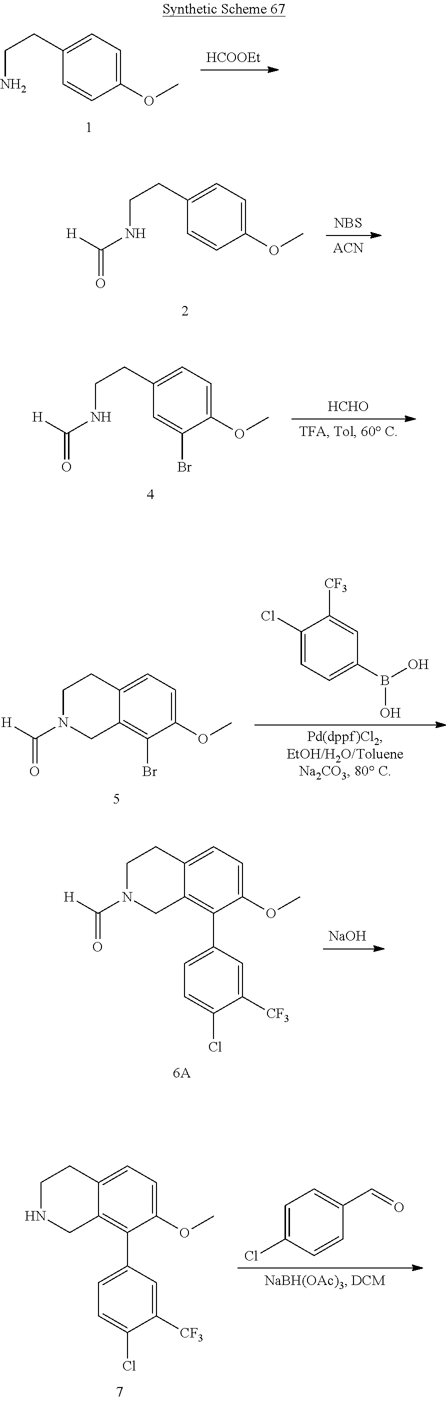

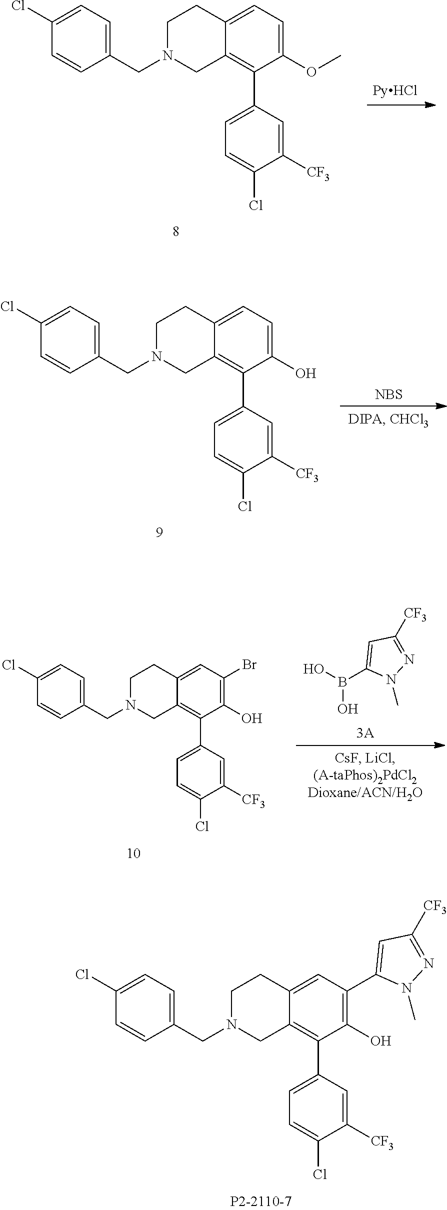

### LCMS Methods:

### Method 1: 50-100AB_6min-220-254-ELSD

LC/MS (The gradient was 50% B in 0.40 min and 50-100% B in 2.60 min, hold on 100% B in 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Kinetex C18 2.1*50 mm, Sum. Detection methods are diode array (DAD), and evaporative light scattering detection (ELSD). MS mode was positive electrospray ionization. MS range was 100-1000.

### Method 2:5-95AB 2 min

LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 ml/min.

### Method 3:5-95CD_2_min

LC/MS (The column used for chromatography was Xbridge C18 2.1*50 mm, 5 um. Detection methods are diode array (DAD). MS mode was positive electrospray ionization. MS range was 50-2000. Mobile phase A was 10 mM Ammonium bicarbonate in water, and mobile phase B was HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### Experimentals for Largest Scale Run

### General Procedure for Preparation of Compound 2-ET42365-532

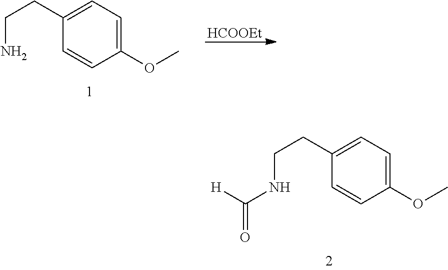

A mixture of Compound 1 (25 g, 165.34 mmol, 24.27 mL, 1 eq) in ethyl formate (230.25 g, 3.11 mol, 250 ml, 18.80 eq) was stirred at 70° C. for 12 hrs. LCMS showed the starting material was consumed and a new peak with desired product Ms was detected. The reaction was concentrated to give Compound 2 (27 g, yield 91.12%) as a yellow solid, which was used to the next step directly without further purification.

$$LCMS ( ESI + ) : RT = 0.674 min , m / z 180.2 {( M + H )}^{+} .$$

5-95CD_2_min: LC/MS (The column used for chromatography was Xbridge C18 2.1*50 mm, Sum. Detection methods are diode array (DAD). MS mode was positive electrospray ionization. MS range was 50-2000. Mobile phase A was 10 mM Ammonium bicarbonate in water, and mobile phase B was HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Compound 4-ET42365-547

The reactions were conducted in parallel but combined for purification.

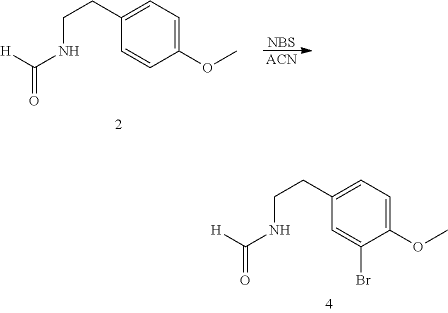

To a solution of Compound 2 (9 g, 50.22 mmol, 1 eq) in AcOH (100 mL) was added NBS (9.83 g, 55.24 mmol, 1.1 eq), the reaction was stirred at 30° C. for 12 hrs. LCMS showed the starting material was consumed and a new peak with desired product Ms was detected. Additional two reactions were set up as described above and all three combined for purification. The combined reaction mixture was concentrated to give a residue, which was diluted with water (600 mL) and was extracted with ethyl acetate (3×450 mL). The combined organic layers were washed with brine (600 mL) and dried over Na₂SO₄ and concentrated to give Compound 4 (30 g. yield 69.43%) as a yellow oil, which was used to the next step directly without further purification.

¹H NMR (ET42365-547-P1A, 400 MHz, CHLOROFORM-d) δ 2.71-2.86 (m, 2H), 3.40-3.62 (m, 2H), 3.89 (s, 3H), 5.39-6.29 (m, 1H), 6.86 (d, J=8.38 Hz, 1H), 7.05-7.16 (m, 1H), 7.35-7.44 (m, 1H), 8.16 (s, 1H)

### General Procedure for Preparation of Compound 5-ET42365-554

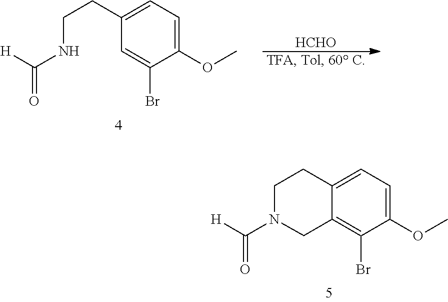

To a solution of Compound 4 (7 g, 27.12 mmol, 1 eq) in toluene (70 mL) were added HCHO (3.26 g, 108.48 mmol, 2.99 mL, 4 eq) and TFA (12.37 g, 108.48 mmol, 8.03 mL, 4 eq), the reaction was stirred at 60° C. for 12 hrs. LCMS showed the starting material was consumed and a new peak with desired product Ms was detected. The reaction was concentrated to give crude product, which was purified by reversed phase column chromatography (Column: 330 g Agela C18, Solvent for sample dissolution about 3.8 gram of sample dissolved in 20 mL of DMF, Flow rate: 70 ml/min, Mobile phase: TFA, Gradient B %: 20-50% 25 mins; 50% 10 mins, Instrument: Biotage) to give Compound 5 (1.4 g, yield 17.2%) as a white solid.

¹H NMR (ET42365-554-P1A, 400 MHz, CHLOROFORM-d) δ 2.73-2.89 (m, 2H), 3.61-3.82 (m, 2H), 3.88 (d, J=1.50 Hz, 3H), 4.48-4.67 (m, 2H), 6.57-6.70 (m, 1H), 7.31-7.38 (m, 1H), 8.19-8.29 (m, 1H)

### General Procedure for Preparation of Compound 6A-ET42365-567

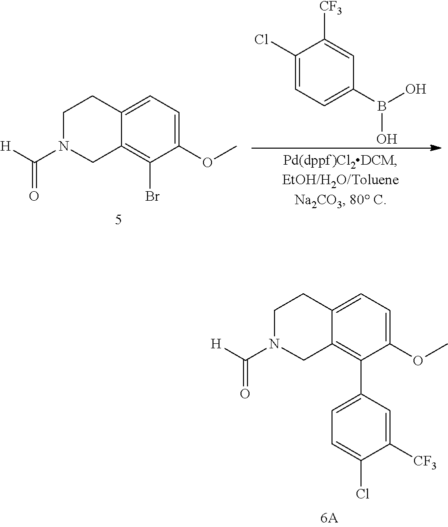 [SMILES: COc1ccc2c(c1Br)CN(C=O)CC2 ; OB(O)c1ccc(Cl)c(C(F)(F)F)c1 ; COc1ccc2c(c1-c1ccc(Cl)c(C(F)(F)F)c1)CN(C=O)CC2]

To a solution of [4-chloro-3-(trifluoromethyl)phenyl]boronic acid (1.40 g, 6.22 mmol, 1.2 eq), Compound 5 (1.4 g, 5.18 mmol, 1 eq) and Na₂CO₃ (1.37 g, 12.96 mmol, 2.5 eq) in a mixture solution of toluene (50 mL), EtOH (10 mL) and H₂O (2 mL) was added Pd(dppf)Cl₂·CH₂Cl₂ (296.28 mg, 362.8 μmol, 0.07 eq) under nitrogen, the reaction was stirred at 80° C. for 12 hrs. LCMS showed the starting material was consumed and a new peak with desired product Ms was detected. The reaction was concentrated to give a residue, which was diluted with water (80 mL) and was extracted with ethyl acetate (3×60 mL). The combined organic layer were washed with brine (80 mL) and dried over Na₂SO₄ and concentrated to give the crude product. The crude product was purified by column chromatography on silica gel (eluted with petroleum ether/THF=5/1 to 1/2) to give Compound 6A (1.4 g, yield 65.75%) was obtained as a yellow solid.

¹H NMR (ET42365-567-P1A, 400 MHz, CHLOROFORM-d) δ 2.82-2.96 (m, 2H), 3.65-3.89 (m, 5H), 4.56-4.78 (m, 2H), 6.69-6.79 (m, 1H), 7.03-7.10 (m, 1H), 7.49-7.55 (m, 1H), 7.59-7.66 (m, 1H), 7.82 (s, 1H), 8.21-8.33 (m, 1H)

### General Procedure for Preparation of Compound 7-ET43587-750

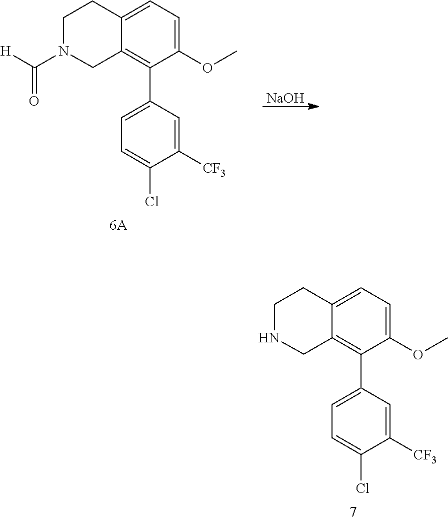 [SMILES: COc1ccc2c(c1-c1ccc(Cl)c(C(F)(F)F)c1)CN(C=O)CC2 ; "NaOH" ; COc1ccc2c(c1-c1ccc(Cl)c(C(F)(F)F)c1)CNCC2]

To a solution of Compound 6A (1 g, 2.70 mmol, 1 eq) in EtOH (10 mL) was added NaOH (324.53 mg, 8.11 mmol, 3 eq), the mixture was stirred at 80° C. for 12 hrs. LCMS showed the starting material was consumed and a new peak with desired product MS was detected. The reaction mixture was filtered and concentrated under reduced pressure to give Compound 7 (500 mg, yield 48.69%) as a brown oil, which was used to the next step without further purification.

$$LCMS ( ESI + ) : RT = 0.704 min , m / z 342.1 {( M + H )}^{+} .$$

5-95AB_2_min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 ml/min.

### General Procedure for Preparation of Compound 8-ET43587-779

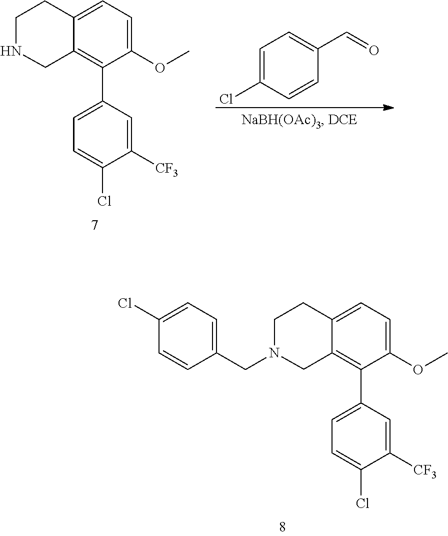 [SMILES: COc1ccc2c(c1-c1ccc(Cl)c(C(F)(F)F)c1)CNCC2 ; O=Cc1ccc(Cl)cc1 ; COc1ccc2c(c1-c1ccc(Cl)c(C(F)(F)F)c1)CN(Cc1ccc(Cl)cc1)CC2]

To a solution of Compound 7 (500 mg, 1.10 mmol, 1 eq. TFA) and 4-chlorobenzaldehyde (616.82 mg, 4.39 mmol, 4 eq) in DCE (10 mL) was stirred at 40° C. for one hour, then NaBH(OAc)₃ (1.16 g, 5.49 mmol, 5 eq) was added at 0° C. The mixture was stirred at 40° C. for 11 hrs. LCMS showed all the starting material was consumed and a new peak with desired product MS was detected. The reaction mixture was diluted with water (20 mL) and extracted with ethyl acetate (3×20 mL). The organic layer was separated and the combined organic layer was washed with brine (2×20 mL), dried over Na₂SO₄, filtered and concentrated under reduced pressure to give a residue, which was purified by Prep-HPLC (column: Phenomenex Luna 80*30 mm*3 um; mobile phase: [water (TFA)-ACN]; B %: 35%-70%, 8 mins) to give Compound 8 (280 mg, yield 54.73%) as a white solid.

¹H NMR (ET43587-779-P1A, 400 MHz, METHANOL-d₄) δ 3.19 (br t. J=5.83 Hz, 2H), 3.42-3.95 (m, 5H), 4.43-4.61 (m, 4H), 6.94 (s, 1H), 7.25 (s, 1H), 7.54-7.61 (m, 4H), 7.61-7.65 (m, 1H), 7.68-7.73 (m, 1H), 7.85 (d, J=1.88 Hz, 1H)

### General Procedure for Preparation of Compound 9-ET43587-814

The reactions were conducted in parallel but combined for purification.

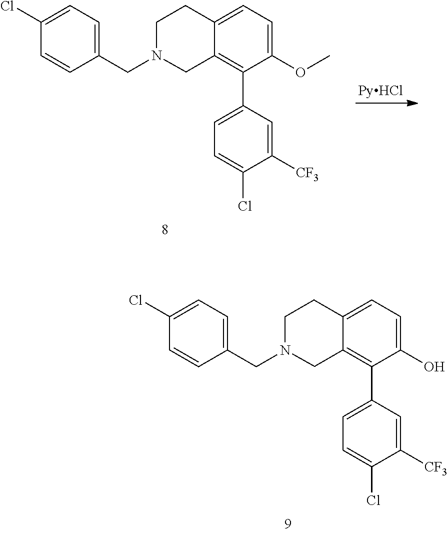 [SMILES: COc1ccc2c(c1-c1ccc(Cl)c(C(F)(F)F)c1)CN(Cc1ccc(Cl)cc1)CC2 ; Oc1ccc2c(c1-c1ccc(Cl)c(C(F)(F)F)c1)CN(Cc1ccc(Cl)cc1)CC2]

A mixture of Compound 8 (140 mg, 300.22 μmol, 1 eq) and pyridine; hydrochloride (2.8 g) was stirred at 180° C. for 6 hrs. LCMS showed all the starting material was consumed and a new peak with desired product MS was detected. Additional one reaction was set up as described above and both two combined for purification. The reaction mixture was diluted with water (30 mL) acidified by NaHCO₃ to pH=7 and extracted with ethyl acetate (3×20 mL). The organic layer was separated and the combined organic layer was washed with brine (2×20 mL), dried over Na₂SO₄, filtered and concentrated under reduced pressure to give a residue, which was purified by chromatography on silica gel (eluted with petroleum ether/ethyl acetate=50/1 to 1/1) to give Compound 9 (130 mg, yield 45.47%) as a yellow oil.

¹H NMR (ET43587-814-p1a, 400 MHz, CHLOROFORM-d) δ 2.61-2.71 (m, 2H), 2.74-2.84 (m, 2H), 3.47-3.61 (m, 4H), 6.46 (s, 1H), 6.93 (s, 1H), 7.21-7.28 (m, 4H), 7.44-7.50 (m, 1H), 7.52-7.57 (m, 1H), 7.75 (d, J=1.88 Hz, 1H)

### General Procedure for Preparation of Compound 10-ET43587-842

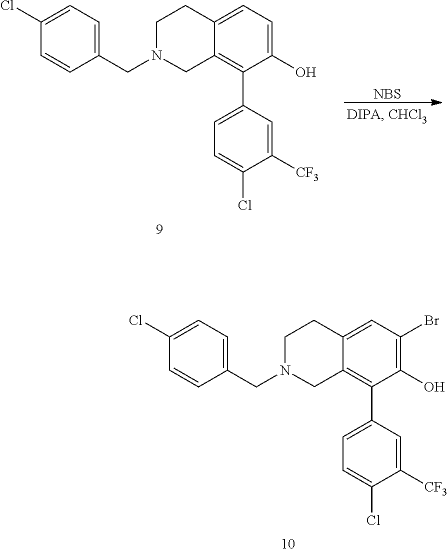 [SMILES: Oc1ccc2c(c1-c1ccc(Cl)c(C(F)(F)F)c1)CN(Cc1ccc(Cl)cc1)CC2 ; "NBS" ; Oc1c(Br)cc2c(c1-c1ccc(Cl)c(C(F)(F)F)c1)CN(Cc1ccc(Cl)cc1)CC2]

To a solution of Compound 9 (130 mg, 287.42 μmol, 1 eq) and DIPA (87.25 mg, 862.27 μmol, 121.86 μL, 3 eq) in CHCl₃ (3 mL) was added NBS (51.16 mg, 287.42 μmol, 1 eq) at −50° C., the mixture was stirred at 20° C. for 12 hrs. LCMS showed all the starting material was consumed and a new peak with desired product MS was detected. The reaction mixture was diluted with water (20 mL) and extracted with DCM (20 mL). The organic layer was separated and the combined organic layer was washed with brine (2×20 mL), dried over Na₂SO₄, filtered and concentrated under reduced pressure to give a residue, which was purified by chromatography on silica gel (eluted with petroleum ether/ethyl acetate=50/1 to 1/1) to give Compound 10 (110 mg, yield 68.45%) as a white solid.

¹H NMR (ET43587-842-P1A, 400 MHz, CHLOROFORM-d) δ 2.67-2.76 (m, 2H), 2.84-2.93 (m, 2H), 3.64 (s, 2H), 3.73 (s, 2H), 5.75 (s, 1H), 7.04 (s, 1H), 7.31-7.39 (m, 4H), 7.55 (d, J=8.41 Hz, 1H), 7.67 (dd, J=8.34, 1.95 Hz, 1H), 7.88 (d, J=1.88 Hz, 1H)

### General Procedure for Preparation of P2-2110-7-ET42365-734

The reactions were conducted in parallel but combined for purification.

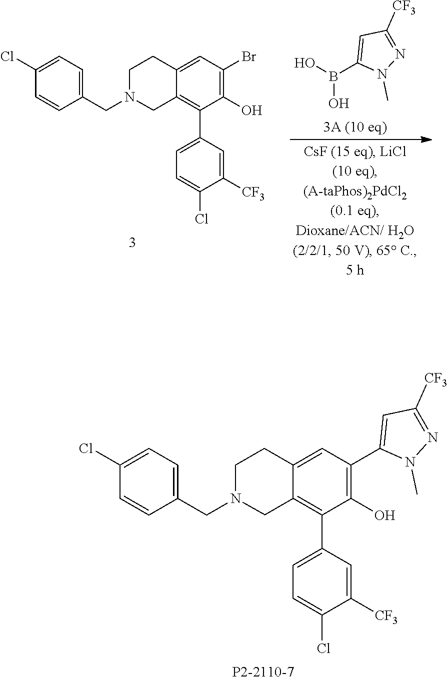 [SMILES: Oc1c(Br)cc2c(c1-c1ccc(Cl)c(C(F)(F)F)c1)CN(Cc1ccc(Cl)cc1)CC2 ; Cn1nc(C(F)(F)F)cc1B(O)O ; Cn1nc(C(F)(F)F)cc1-c1cc2c(c(-c3ccc(Cl)c(C(F)(F)F)c3)c1O)CN(Cc1ccc(Cl)cc1)CC2]

To a solution of Compound 3 (10 mg, 18.83 μmol, 1 eq), Compound 3A (36.51 mg, 188.26 μmol, 10 eq), LiCl (7.98 mg, 188.26 μmol, 3.86 μL, 10 eq) and CsF (42.89 mg, 282.38 μmol, 10.41 μL, 15 eq) in a mixture solution of CH₃CN (0.2 mL), H₂O (0.1 mL) and dioxane (0.2 mL) was added 4-ditert-butylphosphanyl-N,N-dimethyl-aniline;dichloropalladium (1.33 mg, 1.88 μmol, 1.33 μL, 0.1 eq) under nitrogen, the reaction was stirred at 68° C. for 4 hrs. LCMS showed the starting material was consumed, a new peak with desired product Ms was detected and more than 3 peaks. Additional six reactions were set up as described above and all seven combined for purification. The combined reaction was filtered and concentrated to give crude product, which was purified by Prep-HPLC (column: Phenomenex Luna 80*30 mm*3 um; mobile phase: [water (TFA)-ACN]; B %: 35%-65%, 8 mins) to give P2-2110-7 (6 mg, yield 7.58%) as a yellow solid.

¹H NMR (ET42365-734-P1A, 400 MHz, CHLOROFORM-d) δ 2.76-3.57 (m, 4H), 3.59-4.00 (m, 5H), 4.17 (br d, J=12.88 Hz, 1H), 4.36 (br d, J=11.76 Hz, 1H), 6.52 (br s, 1H), 7.25 (br s, 1H), 7.32 (br d, J=8.13 Hz, 2H), 7.43 (br d, J=8.13 Hz, 2H), 7.63 (s, 2H), 7.83 (s, 1H)

$$LCMS ( ESI + ) : RT = 2.576 min , m / z 587. {( M + H )}^{+} .$$

50-100AB_6min-220-254-ELSD: LC/MS (The gradient was 50% B in 0.40 min and 50-100% B in 2.60 min, hold on 100% B in 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Kinetex C18 2.1*50 mm, Sum. Detection methods are diode array (DAD), and evaporative light scattering detection (ELSD). MS mode was positive electrospray ionization. MS range was 100-1000.

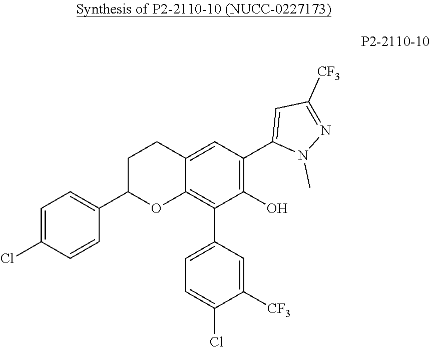 [SMILES: Cn1nc(C(F)(F)F)cc1-c1cc2c(c(-c3ccc(Cl)c(C(F)(F)F)c3)c1O)OC(c1ccc(Cl)cc1)CC2]

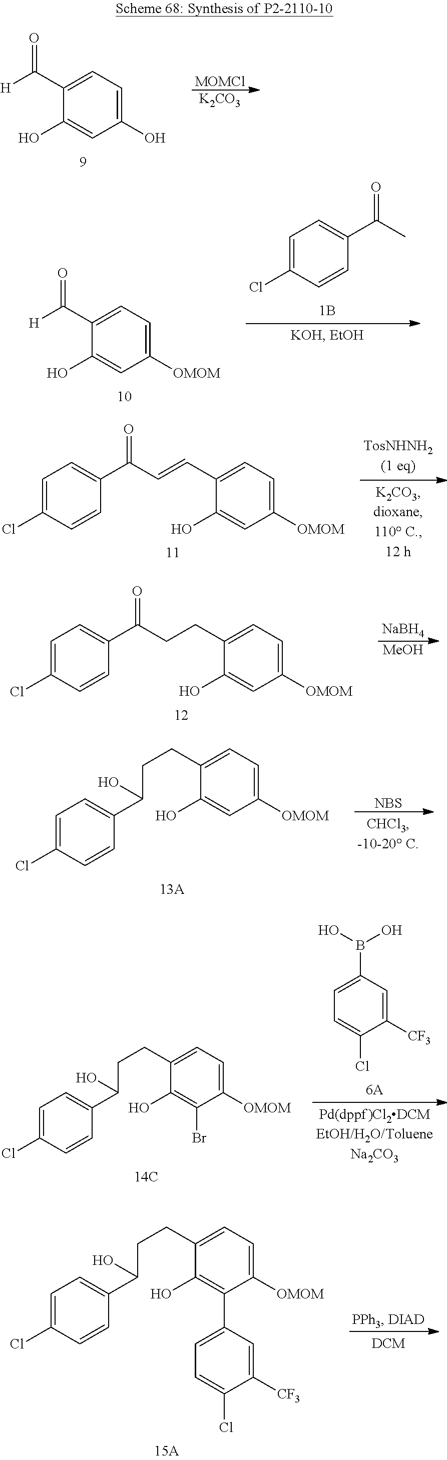

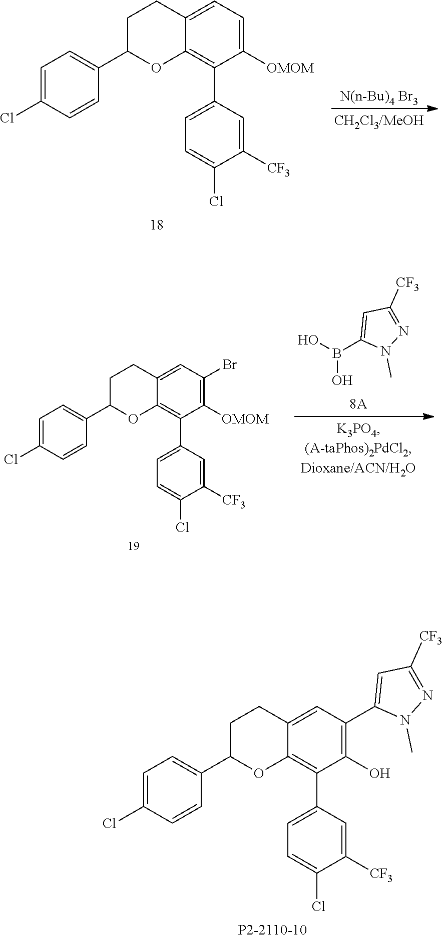

### Chemical Synthesis

### LCMS Methods:

### Method: 50-100AB_6min-220-254-ELSD

LC/MS (The gradient was 50% B in 0.40 min and 50-100% B in 2.60 min, hold on 100% B in 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Kinetex C18 2.1*50 mm, 5 um. Detection methods are diode array (DAD), and evaporative light scattering detection (ELSD). MS mode was positive electrospray ionization. MS range was 100-1000.

### Experimentals for Largest Scale Run

### General Procedure for Preparation of Compound 10-ET51978-68

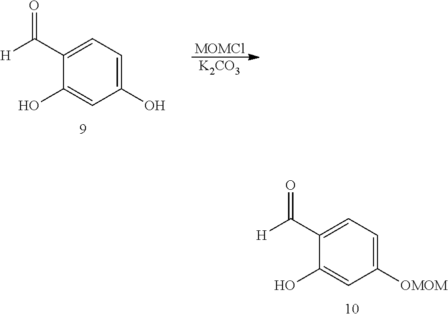

To a solution of Compound 9 (15 g, 108.60 mmol, 1 eq) and K₂CO₃ (19.51 g, 141.18 mmol, 1.3 eq) in acetone (300 mL) was added MOMCl (11.81 g, 146.68 mmol, 11.14 mL, 1.35 eq). The reaction mixture was stirred at 20° C. for 12 hrs. LCMS showed the starting material was consumed and a new peak with desired product MS was detected. The reaction mixture was diluted with water (500 mL) and extracted with ethyl acetate (3×300 mL). The organic layer was separated and the combined organic layer was washed with brine (2×200 mL), dried over Na₂SO₄, filtered and concentrated under reduced pressure to give a residue. The crude product was purified by column chromatography on silica gel (eluted with petroleum ether/tetrahydrofuran=20/1 to 5/1) to give Compound 10 (13.6 g, yield 68.74%) as a yellow solid.

¹H NMR (ET51978-68-P1A, 400 MHz, CHLOROFORM-d) δ 3.49 (s, 3H), 5.23 (s, 2H), 6.58-6.71 (m, 2H), 7.46 (d, J=8.63 Hz, 1H), 9.75 (s, 1H), 11.37 (s, 1H)

### General Procedure for Preparation of Compound 11-ET51978-72

The reactions were conducted in parallel but combined for purification.

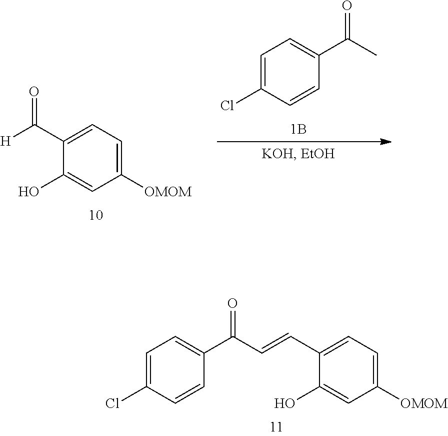

To a solution of Compound 10 (6.8 g, 37.33 mmol, 1 eq) and KOH (4.19 g, 74.65 mmol, 2 eg) in EtOH (135 mL) was added Compound 1B (5.77 g, 37.33 mmol, 4.85 mL, 1 eq). The reaction mixture was stirred at 60° C. for 24 hrs. LCMS showed about 19% of Compound 10 was remaining and a new peak with desired product MS was detected. Additional one reaction was set up as described above and both two combined for purification. The combined reaction mixture was diluted with water (100 mL) and extracted with ethyl acetate (3×100 mL). The organic layer was separated and the combined organic layer was washed with brine (2×100 mL), dried over Na₂SO₄, filtered and concentrated under reduced pressure to give a residue. The crude product was purified by chromatography on silica gel (eluted with petroleum ether/tetrahydrofuran=50/1 to 2/1) to give Compound 11 (15 g, yield 50.43%) as a yellow solid.

¹H NMR (ET51978-72-P1B2, 400 MHz, DMSO-d₆) δ 3.38 (s, 3H), 5.20 (s, 2H), 6.56 (dd, J=8.72, 2.32 Hz, 1H), 6.61 (d, J=2.38 Hz, 1H), 7.59-7.64 (m, 2H), 7.73 (d, J=15.69 Hz, 1H), 7.82 (d, J=8.66 Hz, 1H), 8.00 (d, J=15.69 Hz, 1H), 8.07-8.14 (m, 2H), 10.43 (s, 1H)

### General Procedure for Preparation of Compound 12-ET51978-77

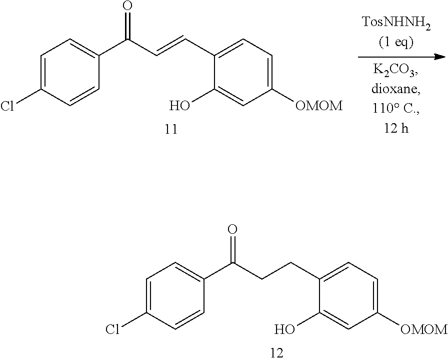

To a solution of Compound 11 (15 g, 47.06 mmol, 1 eq) and K₂CO₃ (39.02 g, 282.35 mmol, 6 eq) in dioxane (104 mL) was added 4-methylbenzenesulfonohydrazide (8.76 g, 47.06 mmol, 1 eq) under nitrogen atmosphere. The reaction mixture was stirred at 110° C. for 12 hrs. LCMS showed starting material was consumed and a new peak with product desired mass was detected. The reaction mixture was diluted with water (300 mL) and extracted with ethyl acetate (3×100 mL). The organic layer was separated and the combined organic layer was washed with brine (2×100 mL), dried over Na₂SO₄, filtered and concentrated under reduced pressure to give a residue. The crude product was purified by chromatography on silica gel (eluted with petroleum ether/tetrahydrofuran=25/1 to 8/1) to give Compound 12 (10.5 g, yield 59.13%) as a yellow solid.

¹H NMR (ET51978-77-P1A, 400 MHz, DMSO-d₆) δ 2.78 (t, J=7.57 Hz, 2H), 3.19-3.26 (m, 2H), 3.35 (s, 3H), 5.08 (s, 2H), 6.38 (dd, J=8.19, 2.44 Hz, 1H), 6.50 (d, J=2.38 Hz, 1H), 6.99 (d, J=8.38 Hz, 1H), 7.58 (d, J=8.63 Hz, 2H), 7.98 (d, J=8.50 Hz, 2H), 9.45 (s, 1H)

### General Procedure for Preparation of Compound 13A-ET51978-81

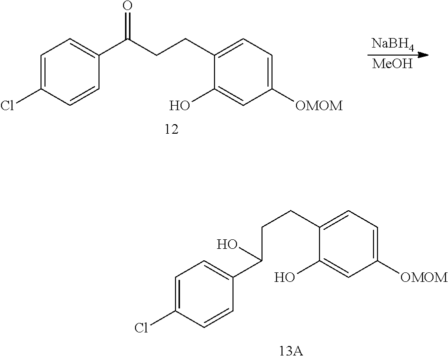

To a solution of Compound 12 (10.5 g, 32.73 mmol, 1 eq) in a mixture solution of tetrahydrofuran (200 mL) and MeOH (200 mL) was added NaBH₄ (1.35 g, 35.68 mmol, 1.09 eq) in portions at 0° C. The reaction mixture was stirred at 20° C. for 1 hour. LCMS showed the starting material was consumed and a new peak with desired product MS was detected. The reaction mixture was poured into saturated aqueous NH₄Cl solution (300 mL) at 0° C. The reaction mixture was concentrated under reduced pressure at 40° C. to remove most of the methanol, and extracted with ethyl acetate (2×200 mL). The organic layer was separated and the combined organic layer was washed with brine (2×200 mL), dried over Na₂SO₄, filtered and concentrated under reduced pressure to give Compound 13A (9 g, yield 76.68%) as a yellow solid, which was used to the next step directly without further purification.

¹H NMR (ET51978-81-P1A, 400 MHz, METHANOL-d₄) δ 1.88-2.00 (m, 2H), 2.47-2.57 (m, 1H), 2.58-2.69 (m, 1H), 3.43 (s, 3H), 4.58 (t, J=6.63 Hz, 1H), 5.09 (s, 2H), 6.39-6.51 (m, 2H), 6.92 (d, J=8.25 Hz, 1H), 7.31 (s, 4H)

### General Procedure for Preparation of Compound 14C-ET42365-657

The reactions were conducted in parallel but combined for purification.

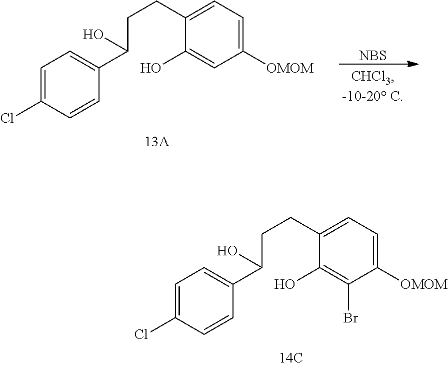

To a solution of Compound 13A (3.5 g, 10.84 mmol, 1 eq) in CHCl₃ (100 mL) was added NBS (1.83 g, 10.30 mmol, 0.95 eq) in portions at 0° C. the reaction was stirred at 20° C. for 12 hrs. LCMS showed the starting material was consumed and a new peak was detected. Additional one reaction was set up as described above and combined for purification. The combined reaction was diluted with water (100 ml) and was extracted with DCM (2×100 mL). The combined organic layer were washed with brine (150 mL) and dried over Na₂SO₄ and concentrated to give crude product. The crude product was purified by silica gel chromatography (eluting with 0 to 10% THF in Petroleum ether) to give Compound 14C (3.5 g, yield 40.18%) as yellow oil.

¹H NMR (ET42365-657-P1A, 400 MHz, METHANOL-d₄) δ 1.91-2.01 (m, 2H), 2.57-2.67 (m, 1H), 2.67-2.78 (m, 1H), 3.47 (s, 3H), 4.59 (t, J=6.69 Hz, 1H), 5.18 (s, 2H), 6.63 (d, J=8.38 Hz, 1H), 6.97 (d, J=8.38 Hz, 1H), 7.28-7.38 (m, 4H)

### General Procedure for Preparation of Compound 15A-ET42365-667

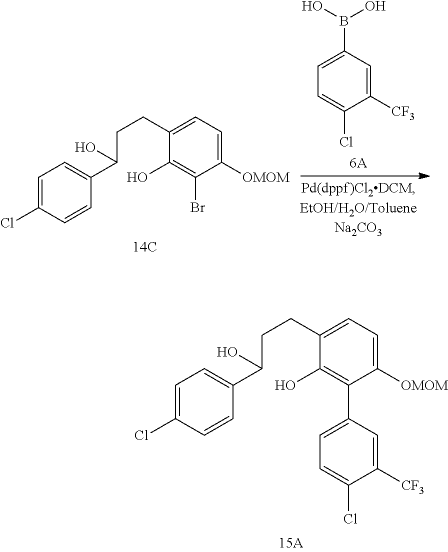

To a solution of Compound 6A (4.19 g, 18.67 mmol, 3 eq), Compound 14C (2.5 g, 6.22 mmol, 1 eq), Na₂CO₃ (2.64 g, 24.90 mmol, 4 eq) and LiCl (2.64 g, 62.24 mmol, 1.27 mL, 10 eq) in a mixture solution of toluene (80 mL), EtOH (16 mL) and H₂O (4 mL) was added Pd(dppf)Cl₂·CH₂Cl₂ (508.27 mg, 622.39 μmol, 0.1 eq) under nitrogen, the reaction was stirred at 82° C. for 12 hrs. LCMS showed about 22% of the starting material was remaining and a new peak with desired product Ms was detected. The reaction was filtered and concentrated to give crude product. The crude product was purified by silica gel chromatography (eluting with 10 to 25% ethyl acetate in petroleum ether) to give Compound 15A (1.1 g, yield 35.25%) as yellow oil.

¹H NMR (ET42365-667-P1A, 400 MHz, CHLOROFORM-d) δ 1.91-2.01 (m, 1H), 2.01-2.12 (m, 1H), 2.26-2.41 (m, 1H), 2.69-2.79 (m, 1H), 2.90 (ddd, J=14.49, 10.04, 6.34 Hz, 1H), 3.35 (s, 3H), 4.66 (dd, J=9.91, 3.76 Hz, 1H), 5.07 (s, 2H), 6.61 (br s, 1H), 6.75 (d, J=8.53 Hz, 1H), 7.09 (d, J=8.41 Hz, 1H), 7.27-7.35 (m, 4H), 7.51-7.56 (m, 1H), 7.56-7.61 (m, 1H), 7.77 (d, J=1.76 Hz, 1H)

### General Procedure for Preparation of 18-ET42365-696

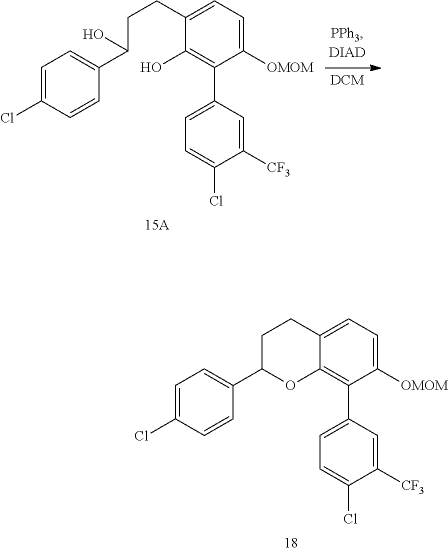

To a solution of Compound 15A (0.6 g, 1.20 mmol, 1 eq) and PPh₃ (627.83 mg, 2.39 mmol, 2 eq) in DCM (12 mL) was added DIAD (484.02 mg, 2.39 mmol, 465.40 μL, 2 eq) dropwise at 0° C., the reaction was stirred at 25° C. for 12 hrs. LCMS showed the starting material was consumed and a new peak with desired product Ms was detected. The reaction was diluted with water (30 mL) and was extracted with ethyl acetate (3×15 mL). The combined organic layer were washed with brine (20 mL) and dried over Na₂SO₄ and concentrated to give crude product. The crude product was purified by silica gel chromatography (eluting with 0 to 15% THF in petroleum ether) to give Compound 18 (0.4 g, yield 69.15%) as a white solid.

¹H NMR (ET42365-696-P1A, 400 MHz, CHLOROFORM-d) δ 1.90-2.07 (m, 1H), 2.25 (br d, J=13.51 Hz, 1H), 2.76-2.89 (m, 1H), 2.93-3.10 (m, 1H), 3.36 (s, 3H), 5.00 (br d, J=9.88 Hz, 1H), 5.07 (s, 2H), 6.80 (br d, J=8.38 Hz, 1H), 7.08 (br d, J=8.38 Hz, 1H), 7.18 (br d, J=8.00 Hz, 2H), 7.30 (br d, J=8.13 Hz, 2H), 7.44-7.59 (m, 2H), 7.82 (s, 1H)

### General Procedure for Preparation of 19-ET42365-701

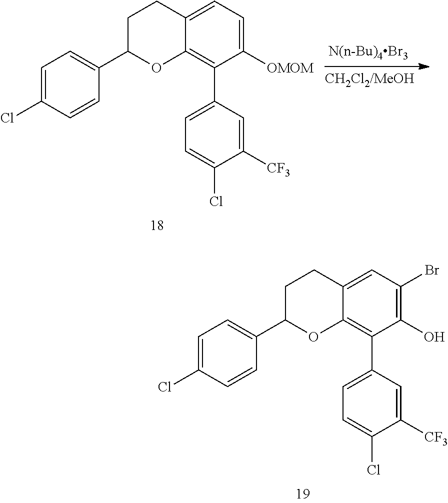

To a solution of Compound 18 (0.4 g, 827.63 μmol, 1 eq) in DCM (5 mL) and MeOH (2.5 mL) was added BLAH;tetrabutylammonium (399.06 mg, 827.63 μmol, 1 eq) at 0° C., the reaction stirred at 30° C. for 12 hrs. LCMS showed the starting material was consumed and a new peak with desired product Ms was detected. The reaction was concentrated to give crude product. The crude product was purified by silica gel chromatography (eluting with 0 to 15% THF in petroleum ether) to give Compound 19 (0.25 g, yield 58.3%) as a yellow solid.

¹H NMR (ET42365-701-P1A, 400 MHz, CHLOROFORM-d) δ 1.91-2.05 (m, 1H), 2.19-2.30 (m, 1H), 2.74-2.84 (m, 1H), 2.91-3.03 (m, 1H), 5.00 (dd, J=10.04, 2.26 Hz, 1H), 5.44 (s, 1H), 7.17 (d, J=8.53 Hz, 2H), 7.26 (s, 1H), 7.28-7.33 (m, 2H), 7.49-7.53 (m, 1H), 7.54-7.58 (m, 1H), 7.83 (d, J=1.63 Hz, 1H)

### General Procedure for Preparation of P2-2110-10-ET42365-705

The reactions were conducted in parallel but combined for purification.

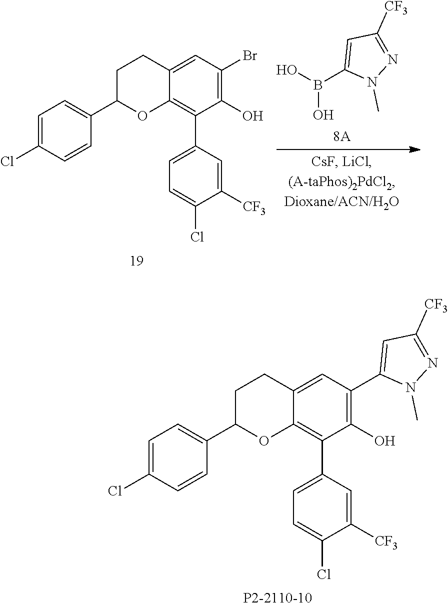 [SMILES: Oc1c(Br)cc2c(c1-c1ccc(Cl)c(C(F)(F)F)c1)OC(c1ccc(Cl)cc1)CC2 ; Cn1nc(C(F)(F)F)cc1B(O)O ; Cn1nc(C(F)(F)F)cc1-c1cc2c(c(-c3ccc(Cl)c(C(F)(F)F)c3)c1O)OC(c1ccc(Cl)cc1)CC2]

To a solution of Compound 19 (40 mg, 77.20 μmol, 1 eq), Compound 8A (104.79 mg, 540.38 μmol, 7 eq), CsF (105.54 mg, 694.78 μmol, 25.62 μL, 9 eq) and LiCl (29.45 mg, 694.78 μmol, 14.23 μL, 9 eq) in a mixture solution of dioxane (0.8 mL), CH₃CN (0.8 mL) and H₂O (0.4 mL) was added 4-ditert-butylphosphanyl-N,N-dimethyl-aniline;dichloro-palladium (5.47 mg, 7.72 μmol, 5.47 μL, 0.1 eq) under nitrogen, the reaction was stirred at 70° C. for 8 hrs. LCMS showed about the starting material was consumed and a new peak with desired product Ms was detected. Additional three reactions were set up as described above and combined for purification. The combined reaction filtered and concentrated to give crude product. The crude product was purified by Prep-HPLC (column: Phenomenex Luna 80*30 mm*3 um; mobile phase: [water (TFA)-ACN]; B %: 75%-85%, 8 mins) to give P2-2110-10 (87.8 mg, yield 47.78%) as off-white solid.

¹H NMR (ET42365-705-P1A, 400 MHz, CHLOROFORM-d) δ 1.96-2.10 (m, 1H), 2.24-2.35 (m, 1H), 2.84 (dt, J=16.09, 4.19 Hz, 1H), 2.94-3.08 (m, 1H), 3.88 (s, 3H), 4.97 (br s, 1H), 5.07 (dd, J=10.10, 2.07 Hz, 1H), 6.58 (s, 1H), 7.03 (s, 1H), 7.18 (d, J=8.41 Hz, 2H), 7.32 (d, J=8.41 Hz, 2H), 7.54-7.63 (m, 2H), 7.84 (d, J=1.51 Hz, 1H)

$$LCMS ( ESI + ) : RT = 2.576 min , m / z 587. {( M + H )}^{+} .$$

50-100AB_6min-220-254-ELSD: LC/MS (The gradient was 50% B in 0.40 min and 50-100% B in 2.60 min, hold on 100% B in 1.00 min, and then 100-50% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Kinetex C18 2.1*50 mm, Sum. Detection methods are diode array (DAD), and evaporative light scattering detection (ELSD). MS mode was positive electrospray ionization. MS range was 100-1000.

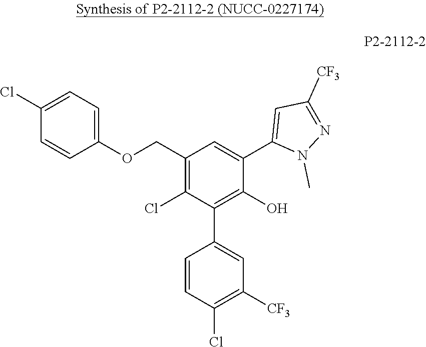 [SMILES: Cn1nc(C(F)(F)F)cc1-c1cc(COc2ccc(Cl)cc2)c(Cl)c(-c2ccc(Cl)c(C(F)(F)F)c2)c1O]

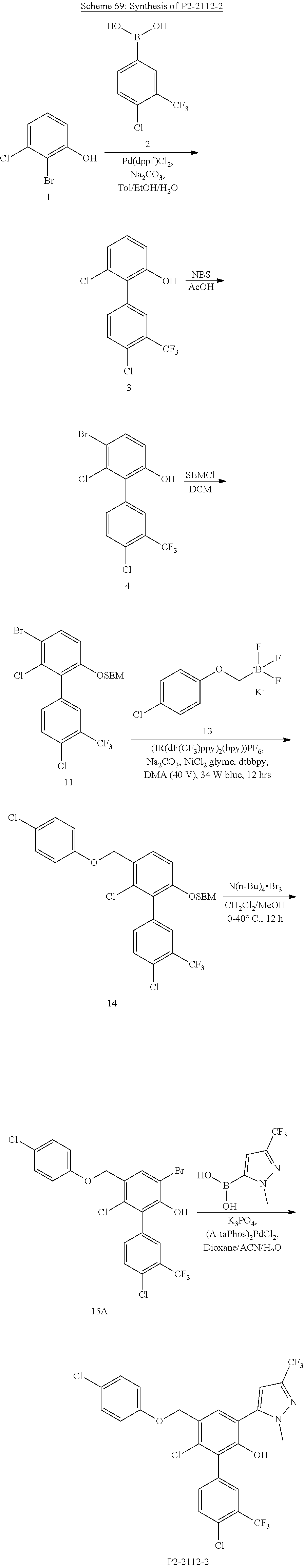

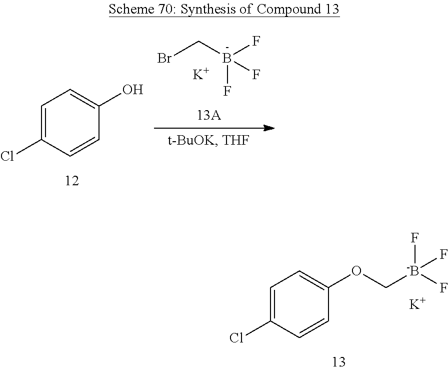

### LCMS Methods:

### Method 1: NEG50_100CD_6min-220-254-ELSD

LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (5 um particles). Detection methods are diode array (DAD) and evaporative light scattering (ELSD) detection. MS mode was negative electrospray ionization. MS range was 100-1000.

### Method 2: NEG50-100CD_2_min

LC/MS (The column used for chromatography was Xbridge C18 2.1*50 mm, 5 um. Detection methods are diode array (DAD). MS mode was negative electrospray ionization. MS range was 50-2000. Mobile phase A was 10 mM ammonium bicarbonate in water, and mobile phase B was HPLC grade acetonitrile. The gradient was 50-100% B in 1.50 min 0.50% B in 0.01 min, 50-100% B (0.01-0.80 min) with a hold at 100% B for 0.40 min, 100-50% B (1.20-1.21 min) with a hold at 50% B for 0.29 min. The flow rate was 1.5 mL/min.

### Experimentals for Largest Scale Run

### General Procedure for Preparation of Compound 3-ET51978-56

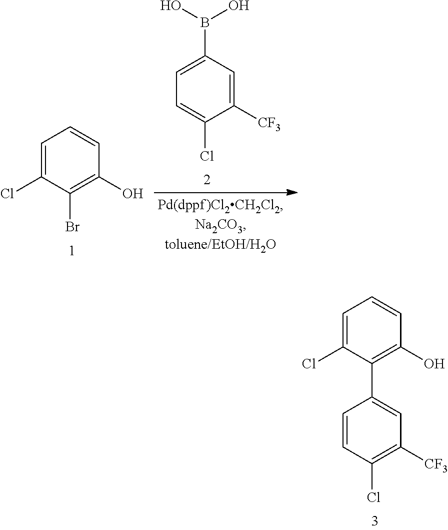 [SMILES: Oc1cccc(Cl)c1Br ; OB(O)c1ccc(Cl)c(C(F)(F)F)c1 ; Oc1cccc(Cl)c1-c1ccc(Cl)c(C(F)(F)F)c1]

To a solution of Compound 1 (10 g, 48.20 mmol, 1 eq), Compound 2 (11.9 g, 53.02 mmol, 1.1 eq) and Na₂CO₃ (10.22 g, 96.40 mmol, 2 eq) in a mixture solution of toluene (300 mL), EtOH (60 mL) and H₂O (15 mL) was added Pd(dppf)Cl₂·CH₂Cl₂ (1.18 g, 1.45 mmol, 0.03 eq) under nitrogen. The reaction mixture was stirred at 85° C. for 12 hrs. LCMS showed about 18% of Compound 1 was remaining and a new peak with desired product MS was detected. The reaction mixture was filtered and concentrated under reduced pressure to give a residue. The residue was diluted with water (300 mL) and extracted with ethyl acetate (3×200 mL). The organic layer was separated and the combined organic layer was washed with brine (2×200 mL), dried over Na₂SO₄, filtered and concentrated under reduced pressure to give a residue. The crude product was purified by chromatography on silica gel (eluted with petroleum ether/ethyl acetate=25/1 to 5/1) to give Compound 3 (9 g, yield 48.64%) as a white solid.

¹H NMR (ET51978-56-P1A, 400 MHz, CHLOROFORM-d) δ 4.79 (s, 1H), 6.90 (br dd, J=8.22, 0.94 Hz, 1H), 7.11 (dd, J=8.09, 0.94 Hz, 1H), 7.20-7.24 (m, 1H), 7.50 (dd, J=8.22, 1.57 Hz, 1H), 7.66 (d, J=8.16 Hz, 1H), 7.71 (d, J=1.51 Hz, 1H)

### General Procedure for Preparation of Compound 4-ET51978-62

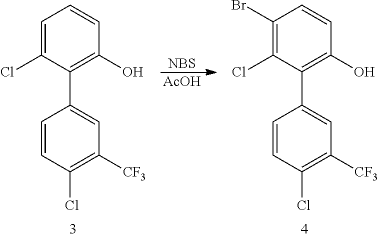

To a solution of Compound 3 (5 g, 16.28 mmol, 1 eq) in AcOH (100 mL) was added NBS (2.90 g, 16.28 mmol, 1 eq) in portions at 0° C. The reaction mixture was added at 25° C. for 2 days. LCMS showed about 12% of Compound 3 was remaining and 31% of product with desired MS was detected. The reaction mixture was filtered and concentrated under reduced pressure to give a crude product, which was purified by reversed phase flash (Column: 120 g Agela C18, Solvent for sample dissolution about 3 grams of sample dissolved in 30 mL of DMF, Flow rate: 50 ml/min, Mobile phase: TFA, Gradient B %: 50-80% 20 mins; 80% 5 mins, Instrument: Biotage) to give Compound 4 (1.3 g, yield 18.62%) as a yellow solid.

¹H NMR (ETS 1978-62-P1A, 400 MHz, CHLOROFORM-d) δ 6.85 (d, J=8.88 Hz, 1H), 7.44-7.47 (m, 1H), 7.57 (d, J=8.75 Hz, 1H), 7.67-7.70 (m, 2H)

### General Procedure for Preparation of Compound 11-ET42365-643

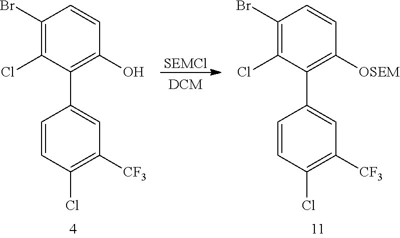

To a solution of Compound 4 (1 g, 2.59 mmol, 1 eq) and DIPEA (1.00 g, 7.77 mmol, 1.35 mL, 3 eq) in DCM (30 mL) was added SEMCl (647.89 mg, 3.89 mmol, 1.5 eq) dropwise at 0° C., the reaction was stirred at 20° C. for 12 hrs. LCMS showed the starting material was consumed and a new peak was detected. The reaction was diluted with water (50 mL) and was extracted with DCM (2×30 mL). The combined organic layer were washed with brine (50 mL) and dried over Na₂SO₄ and concentrated to give crude product. The crude product was purified by Prep-HPLC (column: Waters Xbridge Prep OBD C18 150*40 mm*10 um; mobile phase: [water (NH₄HCO₃)-ACN]; B %: 85%-95%, 8 mins) to give Compound 11 (0.8 g, yield 59.81%) as a colorless oil.

¹H NMR (ET42365-643-P1S3, 400 MHz, CHLOROFORM-d) δ −0.02 (s, 9H), 0.88 (dd, J=8.66, 8.03 Hz, 2H), 3.50-3.64 (m, 2H), 5.11 (s, 2H), 7.07 (d, J=9.03 Hz, 1H), 7.37 (dd, J=8.22, 1.82 Hz, 1H), 7.55-7.66 (m, 3H)

### General Procedure for Preparation of Compound 14-ET42365-680

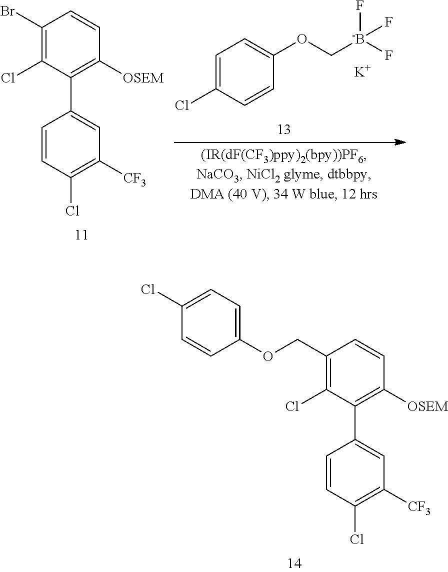

To a solution of Compound 11 (0.5 g, 968.52 μmol, 1 eq) and Compound 13 (481.31 mg, 1.94 mmol, 2 eq) in DMA (8 mL) were added Na₂CO₃ (307.96 mg, 2.91 mmol, 3 eq), bis[3,5-difluoro-2-[5-(trifluoromethyl)-2-pyridyl]phenyl]iridium (1+); 2-(2-pyridyl)pyridine;hexafluorophosphate (48.90 mg, 48.43 μmol, 0.05 eq), 4-tert-butyl-2-(4-tertbutyl-2-pyridyl)-pyridine (25.99 mg, 96.85 μmol, 0.1 eq) and dichloronickel;1,2-dimethoxyethane (21.28 mg, 96.85 μmol, 0.1 eq). The mixture was adjacent to a blue LED (34 W) and stirred at 25° C. for 20 hrs. HPLC showed about 5% of the starting material was remaining and a new peak was detected. The reaction was diluted with water (30 mL) and was extracted with ethyl acetate (3×15 mL). The combined organic layer were washed with brine (20 mL) and dried over Na₂SO₄ and concentrated to give crude product. The crude product was purified by silica gel chromatography (eluting with 0 to 10% ethyl acetate in petroleum ether) to give Compound 14 (250 mg, yield 44.66%) as colorless oil.

¹H NMR (ET42365-680-P1A, 400 MHz, CHLOROFORM-d) δ −0.02 (s, 9H), 0.88 (dd, J=8.85, 7.84 Hz, 2H), 3.51-3.66 (m, 2H), 5.13 (d, J=5.90 Hz, 4H), 6.91-6.97 (m, 2H), 7.18 (d, J=8.66 Hz, 1H), 7.26 (s, 1H), 7.27-7.29 (m, 1H), 7.41 (dd, J=8.22, 1.95 Hz, 1H), 7.51 (d, J=8.66 Hz, 1H), 7.58 (d, J=8.28 Hz, 1H), 7.63 (d, J=1.76 Hz, 1H)

### General Procedure for Preparation of 15A-ET42365-693

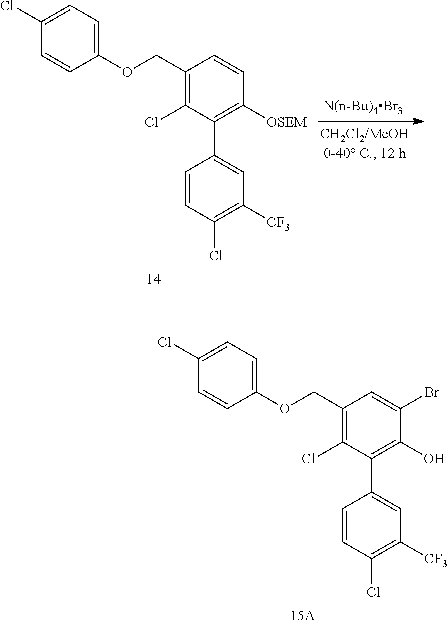

To a solution of Compound 14 (200 mg, 346.07 μmol, 1 eq) in DCM (S mL) and MeOH (2.5 mL) was added BLAH;tetrabutylammonium (166.86 mg, 346.07 μmol, 1 eq) at 0° C., the reaction was stirred at 40° C. for 12 hrs. LCMS showed the starting material was consumed and a new peak with desired product Ms was detected. The reaction was concentrated to give a residue. The crude product was purified by Prep-TLC (petroleum ether/ethyl acetate=5/1) to give Compound 15A (120 mg, yield 65.85%) as a colorless oil.

$$LCMS ( ESI + ) : RT = 0.91 min , m / z 524.8 {( M - H )}^{-} .$$

NEG50-100CD_2_min: LC/MS (The column used for chromatography was Xbridge C18 2 1*50 mm, S um. Detection methods are diode array (DAD). MS mode was negative electrospray ionization. MS range was 50-2000. Mobile phase A was 10 mM ammonium bicarbonate in water, and mobile phase B was HPLC grade acetonitrile. The gradient was 50-100% B in 1.50 min 0.50% B in 0.01 min, 50-100% B (0.01-0.80 min) with a hold at 100% B for 0.40 min, 100-50% B (1.20-1.21 min) with a hold at 50% B for 0.29 min. The flow rate was 1.5 mL/min.

### General Procedure for Preparation of P2-2112-2-ET42365-706

The reactions were conducted in parallel but combined for purification.

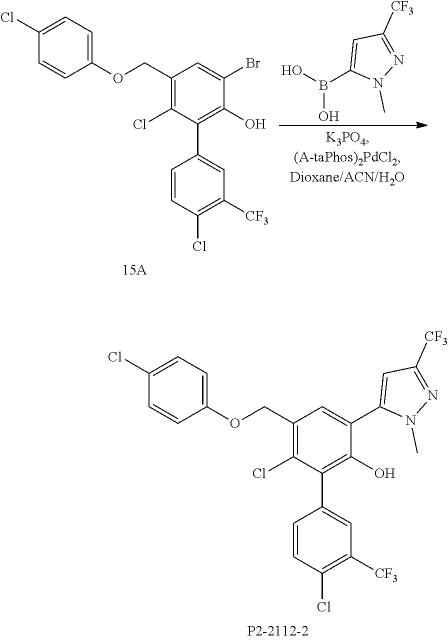 [SMILES: Oc1c(Br)cc(COc2ccc(Cl)cc2)c(Cl)c1-c1ccc(Cl)c(C(F)(F)F)c1 ; Cn1nc(C(F)(F)F)cc1B(O)O ; Cn1nc(C(F)(F)F)cc1-c1cc(COc2ccc(Cl)cc2)c(Cl)c(-c2ccc(Cl)c(C(F)(F)F)c2)c1O]

To a solution of Compound 15A (30 mg, 56.97 μmol, 1 eq). [2-methyl-5-(trifluoro ethyl) pyrazol-3-yl]boronic acid (55.24 mg, 284.87 μmol, 5 eq), CsF (60.58 mg, 398.82 μmol, 14.70 μL, 7 eq) and LiCl (16.91 mg, 398.82 μmol, 8.17 μL, 7 eq) in a mixture solution of dioxane (0.6 mL), CH₃CN (0.6 mL) and H₂O (0.3 mL) was added 4-ditert-butylphosphanyl-N,N-dimethyl-aniline;dichloropalladium (4.03 mg, 5.70 μmol, 4.03 μL, 0.1 eq) under nitrogen, the reaction was stirred at 68° C. for 10 hrs. LCMS showed the Starting material was consumed, a new peak with desired product Ms was detected and more than 5 small peaks. Additional three reactions were set up as described above and all four combined for purification. The combined reaction mixture was filtered and concentrated to give crude product. The crude product was purified by Pre-HPLC (column: Phenomenex Luna 80*30 mm*3 um; mobile phase: [water (TFA)-ACN]; B %: 70%-100%, 8 mins) to give P2-2112-2 (29.1 mg, yield 21.43%) as off-white solid.

¹H NMR (ET42365-706-P1A, 400 MHz, CHLOROFORM-d) δ 3.83 (s, 3H), 5.08 (br s, 1H), 5.14 (s, 2H), 6.63 (s, 1H), 6.91-6.98 (m, 2H), 7.29 (d, J=9.03 Hz, 2H), 7.49-7.55 (m, 2H), 7.69-7.76 (m, 2H)

$$LCMS ( ESI + ) : RT = 2.879 min , m / z 593. , 595. {( M - H )}^{-} , {( M + 2 - H )}^{-}$$

NEG50_100CD_6min-220-254-ELSD: LC/MS (The gradient was 50% B in 0.40 min and 50-100% B at 0.40-3.40 min, hold on 100% B for 0.45 min, and then 100-50% B in 0.01 min, the flow rate was 0.8 ml/min. Mobile phase A was H₂O+10 mM NH₄HCO₃, mobile phase B was Acetonitrile. The column used for chromatography was a Xbridge C18 2.1*50 mm column (5 um particles). Detection methods are diode array (DAD) and evaporative light scattering (ELSD) detection. MS mode was negative electrospray ionization. MS range was 100-1000.

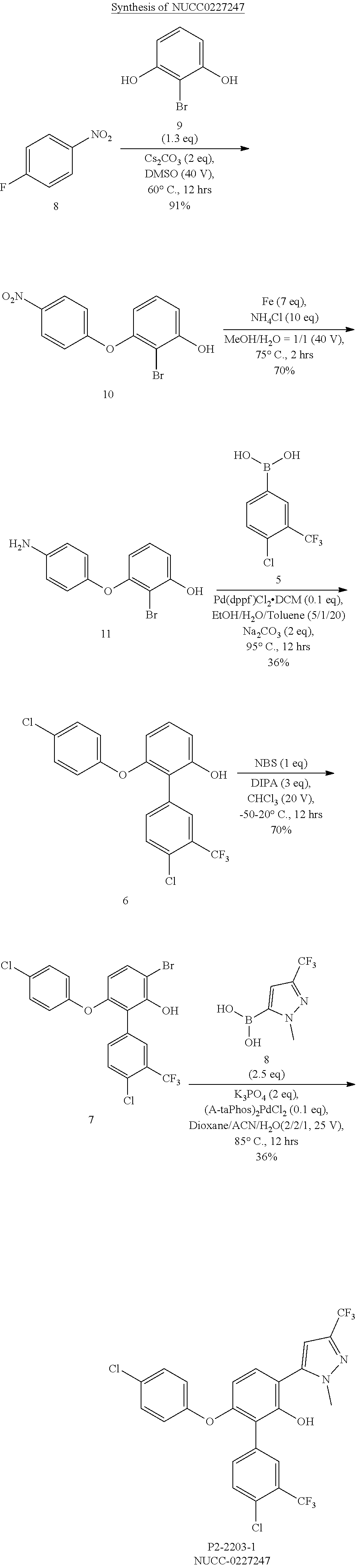

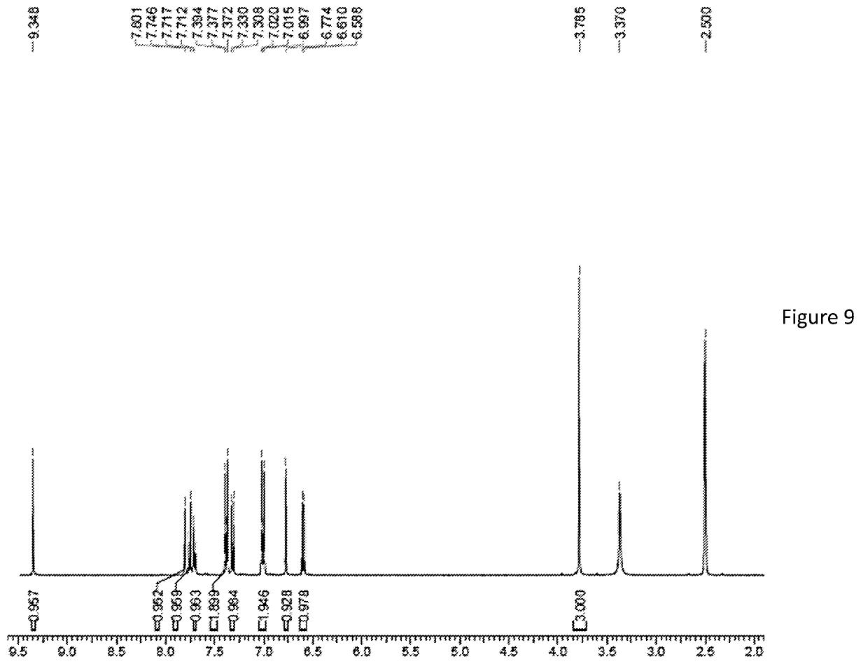

For ¹H NMR of NUCC-0227247 (ie. P2-2203-1), see FIG. 9. ICMS data not shown

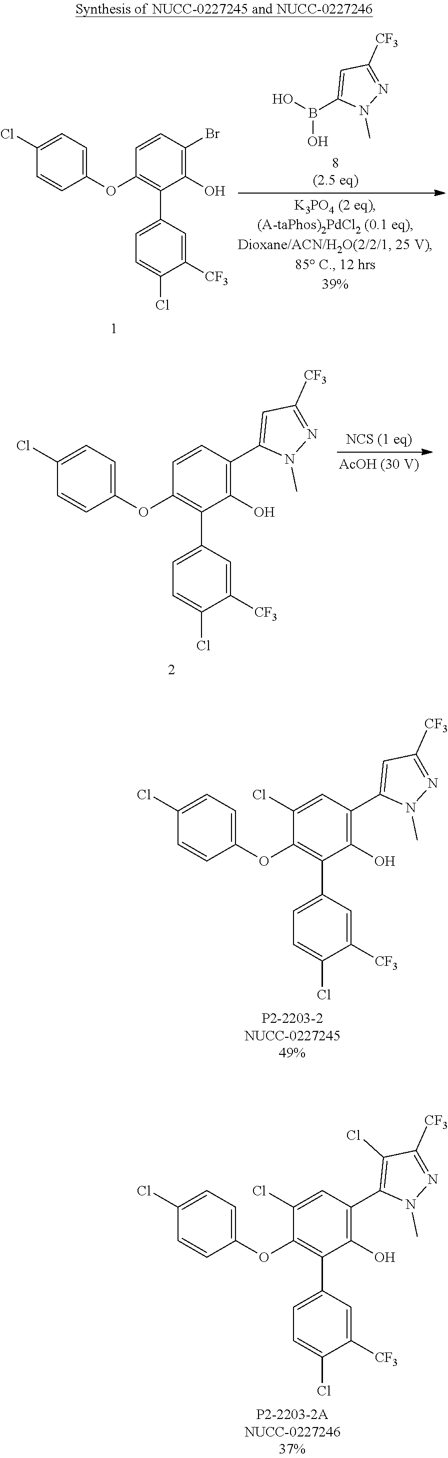 [SMILES: Oc1c(Br)ccc(Oc2ccc(Cl)cc2)c1-c1ccc(Cl)c(C(F)(F)F)c1 ; Cn1nc(C(F)(F)F)cc1B(O)O ; Cn1nc(C(F)(F)F)cc1-c1ccc(Oc2ccc(Cl)cc2)c(-c2ccc(Cl)c(C(F)(F)F)c2)c1O ; Cn1nc(C(F)(F)F)cc1-c1cc(Cl)c(Oc2ccc(Cl)cc2)c(-c2ccc(Cl)c(C(F)(F)F)c2)c1O ; Cn1nc(C(F)(F)F)c(Cl)c1-c1cc(Cl)c(Oc2ccc(Cl)cc2)c(-c2ccc(Cl)c(C(F)(F)F)c2)c1O]

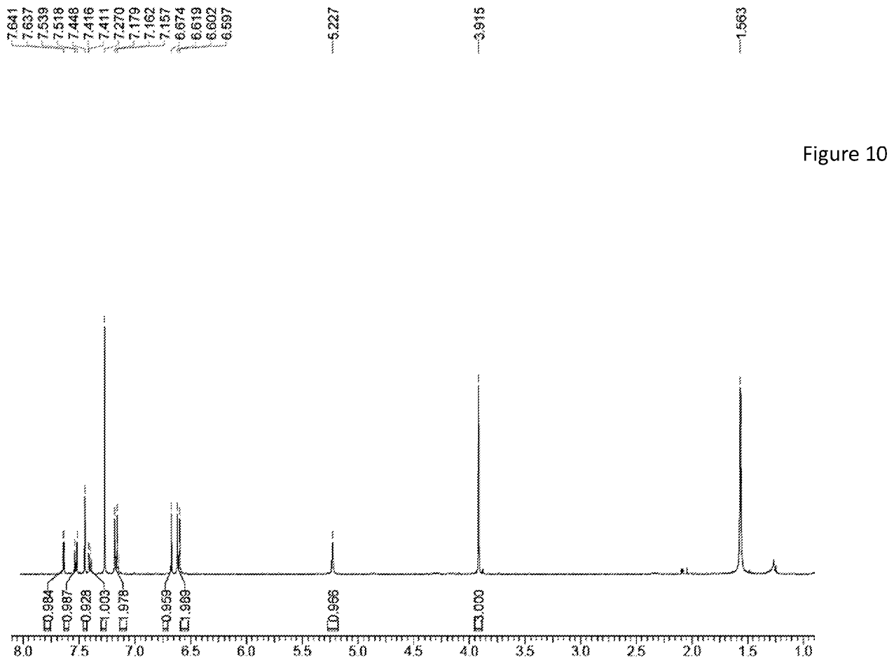

For ¹H NMR of NUCC-0227245 (i.e. P2-2203-2), see FIG. 10, LCMS data not shown.

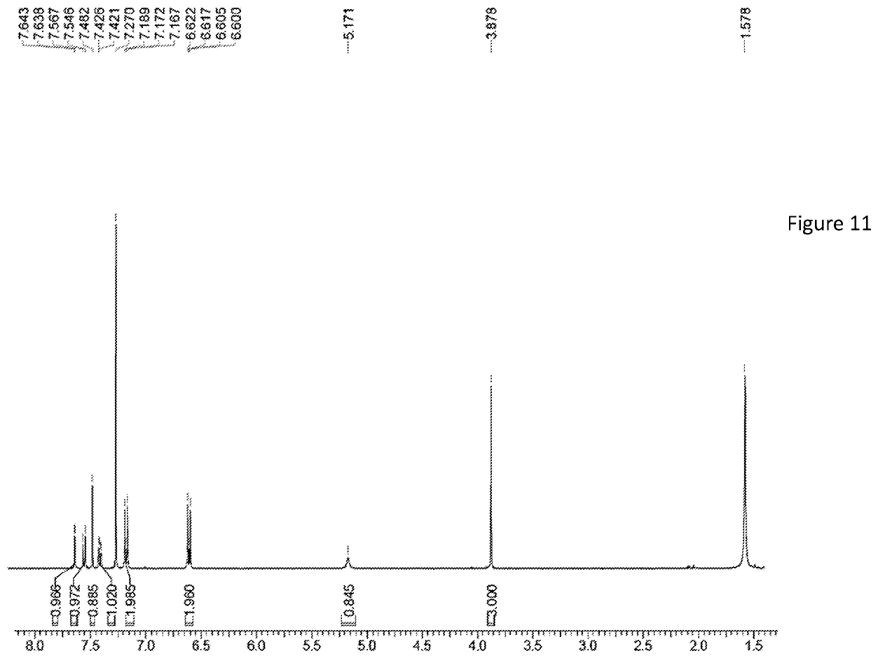

For ¹H NMR of NUCC-0227246 (i.e., P2-2203-2A), see FIG. 11. LCMS data not shown.

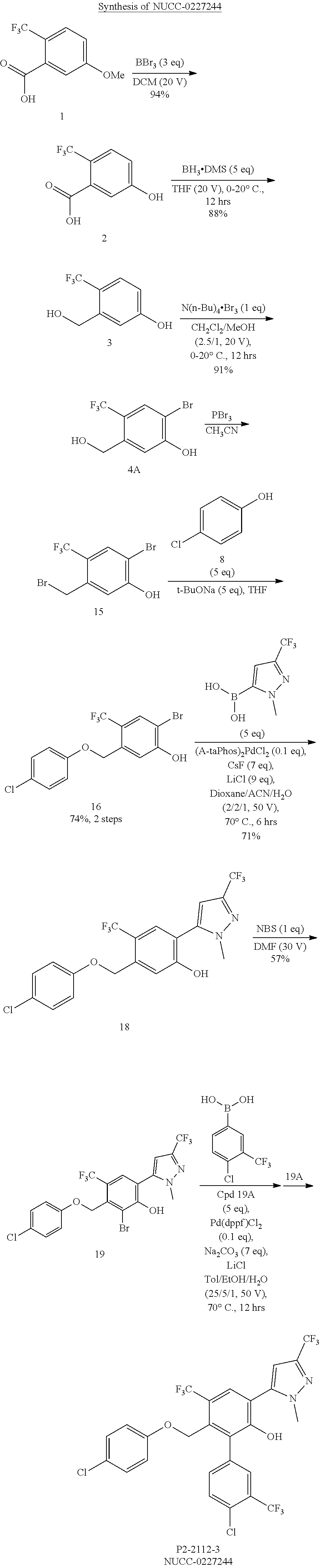

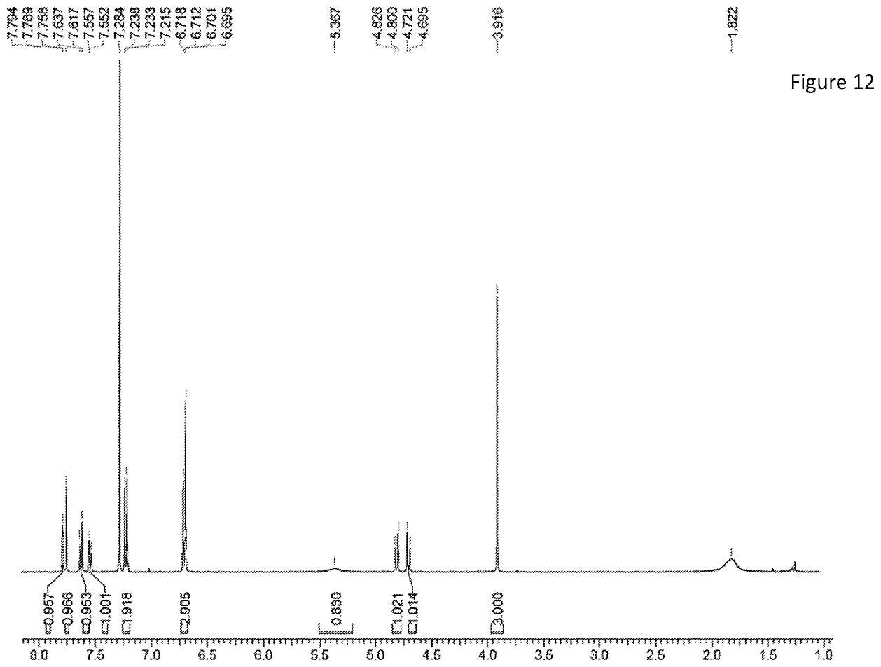

For ¹H NMR of NUCC-0227244 (i.e., P2-2112-3), see FIG. 12. LCMS data not shown.

### Table 1

| Molecule Name | Structure |
|---|---|
| NUCC-0227066 | 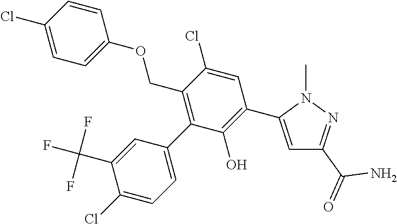 [SMILES: Cn1nc(C(N)=O)cc1-c1cc(Cl)c(COc2ccc(Cl)cc2)c(-c2ccc(Cl)c(C(F)(F)F)c2)c1O] |
| NUCC-0227065 | 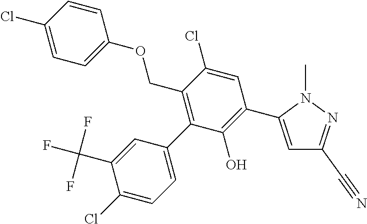 [SMILES: Cn1nc(C#N)cc1-c1cc(Cl)c(COc2ccc(Cl)cc2)c(-c2ccc(Cl)c(C(F)(F)F)c2)c1O] |
| NUCC-0227064 | 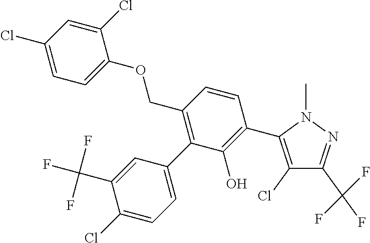 [SMILES: Cn1nc(C(F)(F)F)c(Cl)c1-c1ccc(COc2ccc(Cl)cc2Cl)c(-c2ccc(Cl)c(C(F)(F)F)c2)c1O] |
| NUCC-0227063 | 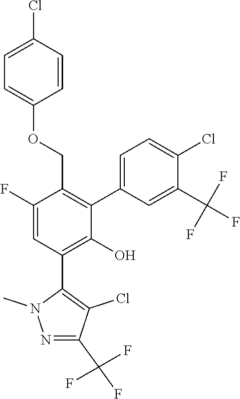 [SMILES: Cn1nc(C(F)(F)F)c(Cl)c1-c1cc(F)c(COc2ccc(Cl)cc2)c(-c2ccc(Cl)c(C(F)(F)F)c2)c1O] |
| NUCC-0227062 | 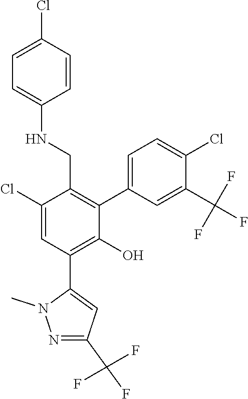 [SMILES: Cn1nc(C(F)(F)F)cc1-c1cc(Cl)c(CNc2ccc(Cl)cc2)c(-c2ccc(Cl)c(C(F)(F)F)c2)c1O] |
| NUCC-0227038 | 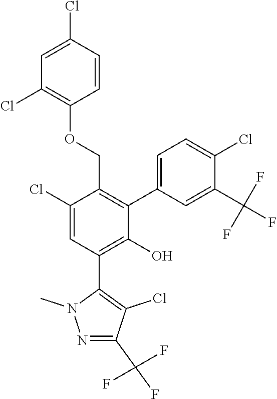 [SMILES: Cn1nc(C(F)(F)F)c(Cl)c1-c1cc(Cl)c(COc2ccc(Cl)cc2Cl)c(-c2ccc(Cl)c(C(F)(F)F)c2)c1O] |
| NUCC-0227037 | 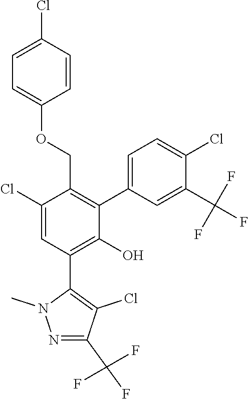 [SMILES: Cn1nc(C(F)(F)F)c(Cl)c1-c1cc(Cl)c(COc2ccc(Cl)cc2)c(-c2ccc(Cl)c(C(F)(F)F)c2)c1O] |
| NUCC-0226915 | 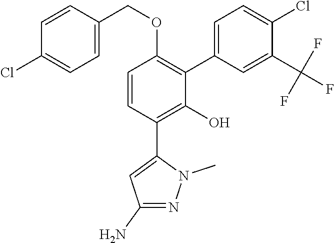 [SMILES: Cn1nc(N)cc1-c1ccc(OCc2ccc(Cl)cc2)c(-c2ccc(Cl)c(C(F)(F)F)c2)c1O] |
| NUCC-0226914 | 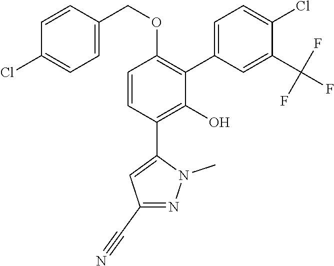 [SMILES: Cn1nc(C#N)cc1-c1ccc(OCc2ccc(Cl)cc2)c(-c2ccc(Cl)c(C(F)(F)F)c2)c1O] |
| NUCC-0226661 | 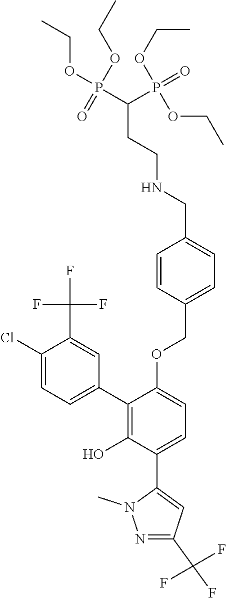 [SMILES: CCOP(=O)(OCC)C(CCNCc1ccc(COc2ccc(-c3cc(C(F)(F)F)nn3C)c(O)c2-c2ccc(Cl)c(C(F)(F)F)c2)cc1)P(=O)(OCC)OCC] |
| NUCC- 0226660 | 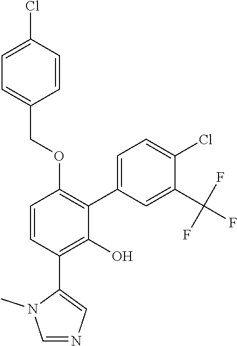 [SMILES: Cn1cncc1-c1ccc(OCc2ccc(Cl)cc2)c(-c2ccc(Cl)c(C(F)(F)F)c2)c1O] |
| NUCC- 0226659 | 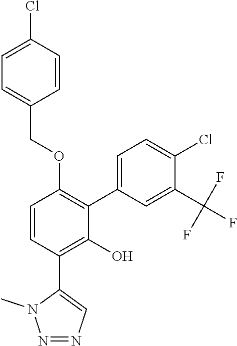 [SMILES: Cn1nncc1-c1ccc(OCc2ccc(Cl)cc2)c(-c2ccc(Cl)c(C(F)(F)F)c2)c1O] |
| NUCC-0226658 | 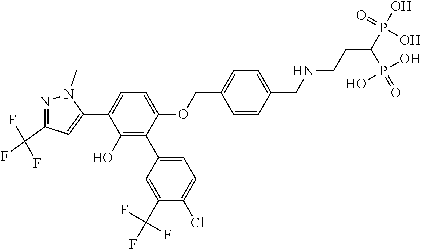 [SMILES: Cn1nc(C(F)(F)F)cc1-c1ccc(OCc2ccc(CNCCC(P(=O)(O)O)P(=O)(O)O)cc2)c(-c2ccc(Cl)c(C(F)(F)F)c2)c1O] |
| NUCC-0226657 | 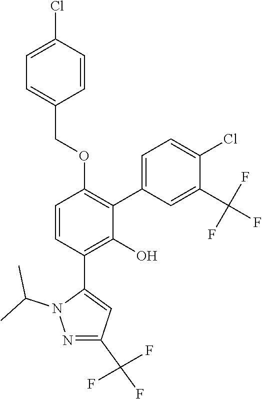 [SMILES: CC(C)n1nc(C(F)(F)F)cc1-c1ccc(OCc2ccc(Cl)cc2)c(-c2ccc(Cl)c(C(F)(F)F)c2)c1O] |
| NUCC-0226656 | 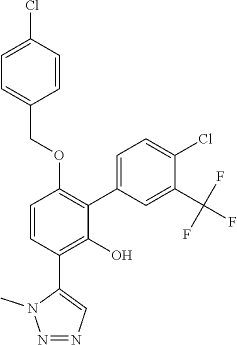 [SMILES: Cn1nncc1-c1ccc(OCc2ccc(Cl)cc2)c(-c2ccc(Cl)c(C(F)(F)F)c2)c1O] |
| NUCC-0226652 | 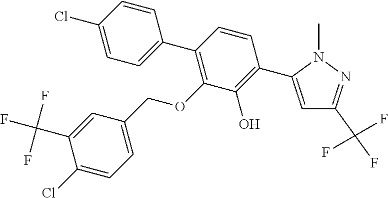 [SMILES: Cn1nc(C(F)(F)F)cc1-c1ccc(-c2ccc(Cl)cc2)c(OCc2ccc(Cl)c(C(F)(F)F)c2)c1O] |
| NUCC-0226606 | 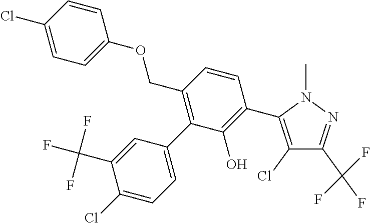 [SMILES: Cn1nc(C(F)(F)F)c(Cl)c1-c1ccc(COc2ccc(Cl)cc2)c(-c2ccc(Cl)c(C(F)(F)F)c2)c1O] |
| NUCC-0226605 | 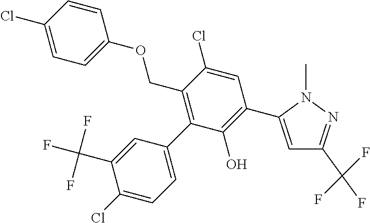 [SMILES: Cn1nc(C(F)(F)F)cc1-c1cc(Cl)c(COc2ccc(Cl)cc2)c(-c2ccc(Cl)c(C(F)(F)F)c2)c1O] |
| NUCC-0226604 | 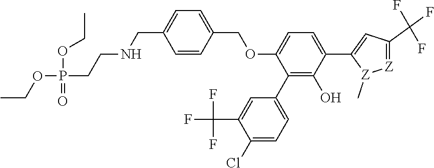 |
| NUCC-0226603 | 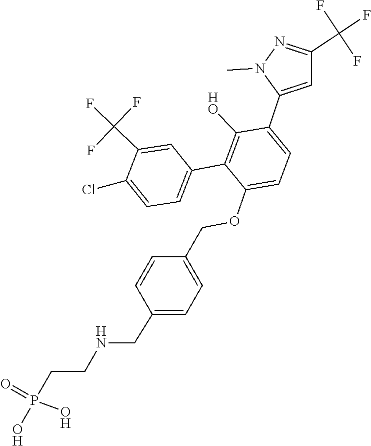 [SMILES: Cn1nc(C(F)(F)F)cc1-c1ccc(OCc2ccc(CNCCP(=O)(O)O)cc2)c(-c2ccc(Cl)c(C(F)(F)F)c2)c1O] |
| NUCC-0226597 | 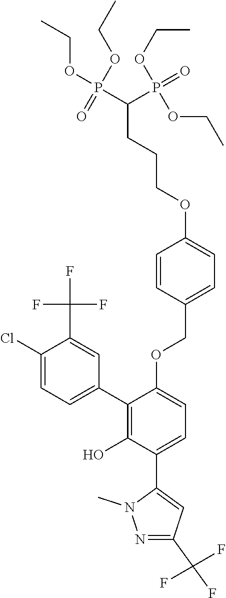 [SMILES: CCOP(=O)(OCC)C(CCCOc1ccc(COc2ccc(-c3cc(C(F)(F)F)nn3C)c(O)c2-c2ccc(Cl)c(C(F)(F)F)c2)cc1)P(=O)(OCC)OCC] |
| NUCC-0226596 | 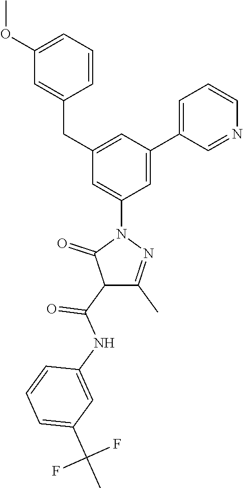 [SMILES: COc1cccc(Cc2cc(-c3cccnc3)cc(N3N=C(C)C(C(=O)Nc4cccc(C(C)(F)F)c4)C3=O)c2)c1] |
| NUCC-0226595 | 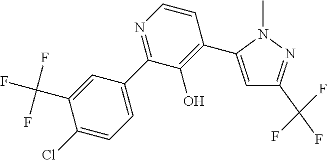 [SMILES: Cn1nc(C(F)(F)F)cc1-c1ccnc(-c2ccc(Cl)c(C(F)(F)F)c2)c1O] |
| NUCC-0226574 | 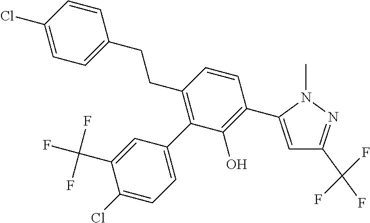 [SMILES: Cn1nc(C(F)(F)F)cc1-c1ccc(CCc2ccc(Cl)cc2)c(-c2ccc(Cl)c(C(F)(F)F)c2)c1O] |
| NUCC-0226566 | 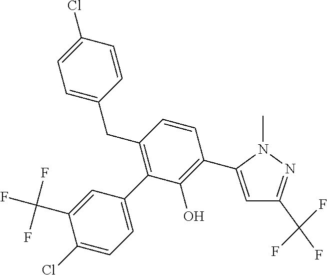 [SMILES: Cn1nc(C(F)(F)F)cc1-c1ccc(Cc2ccc(Cl)cc2)c(-c2ccc(Cl)c(C(F)(F)F)c2)c1O] |
| NUCC-0226548 | 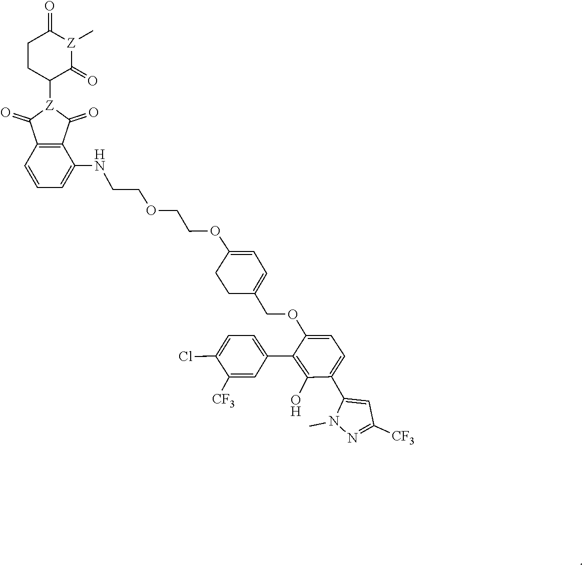 |
| NUCC-0226545 | 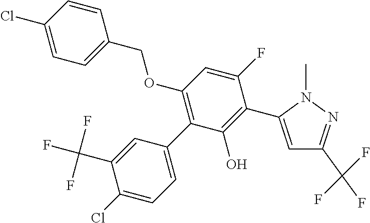 [SMILES: Cn1nc(C(F)(F)F)cc1-c1c(F)cc(OCc2ccc(Cl)cc2)c(-c2ccc(Cl)c(C(F)(F)F)c2)c1O] |
| NUCC-0226530 | 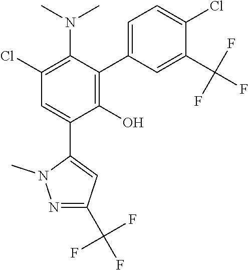 [SMILES: CN(C)c1c(Cl)cc(-c2cc(C(F)(F)F)nn2C)c(O)c1-c1ccc(Cl)c(C(F)(F)F)c1] |
| NUCC-0226529 | 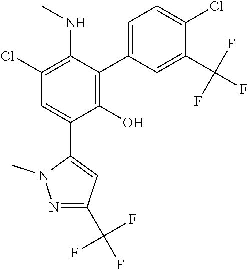 [SMILES: CNc1c(Cl)cc(-c2cc(C(F)(F)F)nn2C)c(O)c1-c1ccc(Cl)c(C(F)(F)F)c1] |
| NUCC-0226528 | 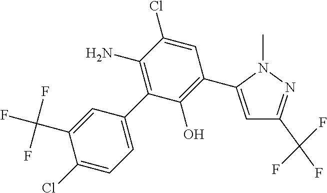 [SMILES: Cn1nc(C(F)(F)F)cc1-c1cc(Cl)c(N)c(-c2ccc(Cl)c(C(F)(F)F)c2)c1O] |
| NUCC-0226527 | 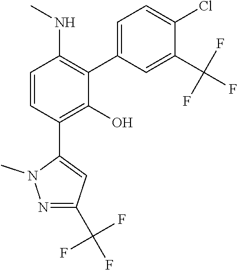 [SMILES: CNc1ccc(-c2cc(C(F)(F)F)nn2C)c(O)c1-c1ccc(Cl)c(C(F)(F)F)c1] |
| NUCC-0226520 | 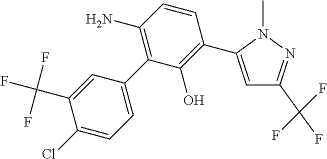 [SMILES: Cn1nc(C(F)(F)F)cc1-c1ccc(N)c(-c2ccc(Cl)c(C(F)(F)F)c2)c1O] |
| NUCC-0226504 | 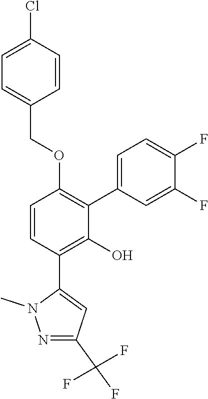 [SMILES: Cn1nc(C(F)(F)F)cc1-c1ccc(OCc2ccc(Cl)cc2)c(-c2ccc(F)c(F)c2)c1O] |
| NUCC-0226503 | 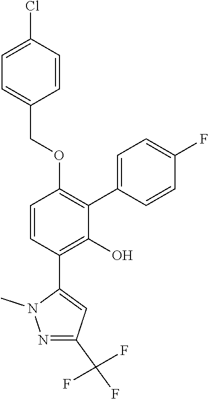 [SMILES: Cn1nc(C(F)(F)F)cc1-c1ccc(OCc2ccc(Cl)cc2)c(-c2ccc(F)cc2)c1O] |
| NUCC-0226502 | 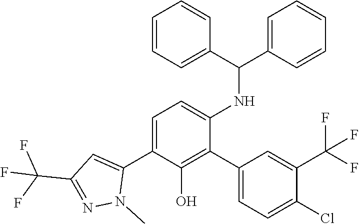 [SMILES: Cn1nc(C(F)(F)F)cc1-c1ccc(NC(c2ccccc2)c2ccccc2)c(-c2ccc(Cl)c(C(F)(F)F)c2)c1O] |
| NUCC-0226501 | 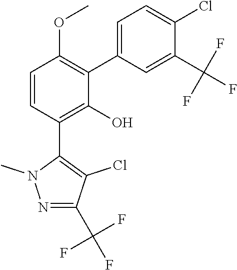 [SMILES: COc1ccc(-c2c(Cl)c(C(F)(F)F)nn2C)c(O)c1-c1ccc(Cl)c(C(F)(F)F)c1] |
| NUCC-0226500 | 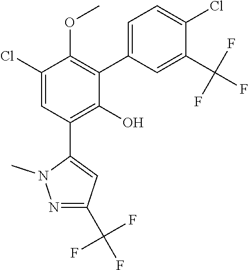 [SMILES: COc1c(Cl)cc(-c2cc(C(F)(F)F)nn2C)c(O)c1-c1ccc(Cl)c(C(F)(F)F)c1] |
| NUCC-0226499 | 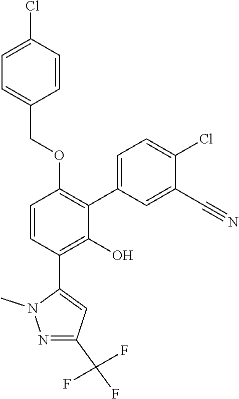 [SMILES: Cn1nc(C(F)(F)F)cc1-c1ccc(OCc2ccc(Cl)cc2)c(-c2ccc(Cl)c(C#N)c2)c1O] |
| NUCC-0226498 | 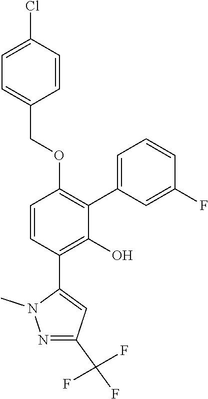 [SMILES: Cn1nc(C(F)(F)F)cc1-c1ccc(OCc2ccc(Cl)cc2)c(-c2cccc(F)c2)c1O] |
| NUCC-0226497 | 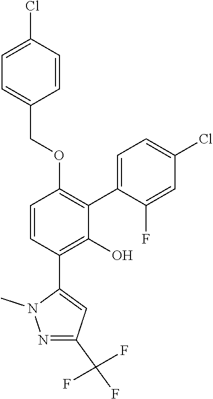 [SMILES: Cn1nc(C(F)(F)F)cc1-c1ccc(OCc2ccc(Cl)cc2)c(-c2ccc(Cl)cc2F)c1O] |
| NUCC-0226496 | 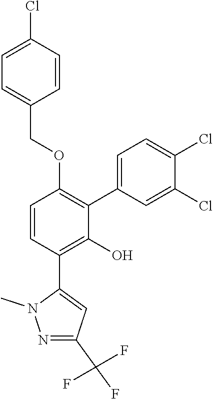 [SMILES: Cn1nc(C(F)(F)F)cc1-c1ccc(OCc2ccc(Cl)cc2)c(-c2ccc(Cl)c(Cl)c2)c1O] |
| NUCC-0226495 | 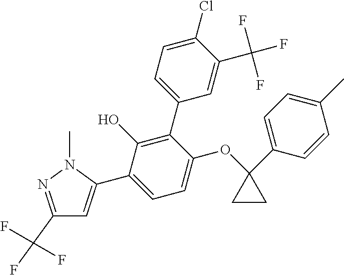 [SMILES: Cc1ccc(C2(Oc3ccc(-c4cc(C(F)(F)F)nn4C)c(O)c3-c3ccc(Cl)c(C(F)(F)F)c3)CC2)cc1] |
| NUCC-0226438 | 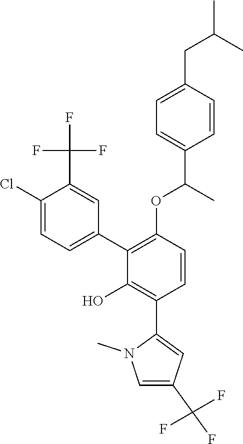 [SMILES: CC(C)Cc1ccc(C(C)Oc2ccc(-c3cc(C(F)(F)F)cn3C)c(O)c2-c2ccc(Cl)c(C(F)(F)F)c2)cc1] |
| NUCC-0226437 | 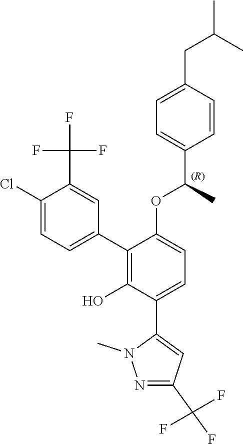 |
| NUCC-0226436 | 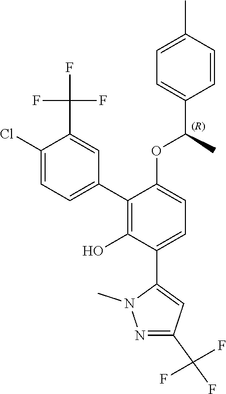 |
| NUCC-0226435 | 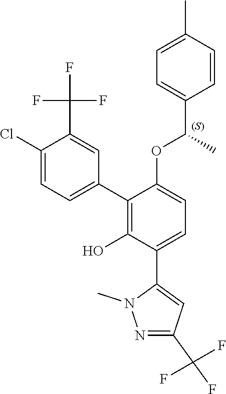 |
| NUCC-0226434 | 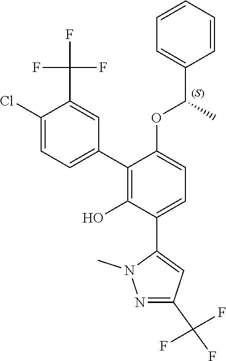 |
| NUCC-0226433 | 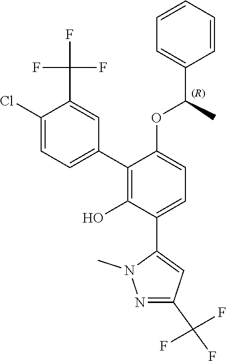 |
| NUCC-0226410 | 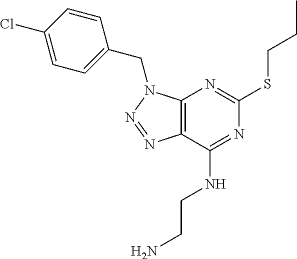 [SMILES: CCCSc1nc(NCCN)c2nnn(Cc3ccc(Cl)cc3)c2n1] |
| NUCC-0226305 | 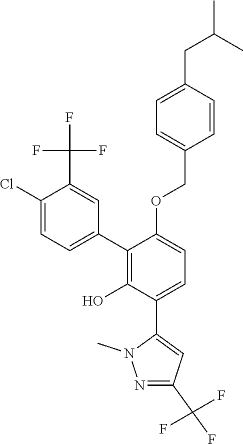 [SMILES: CC(C)Cc1ccc(COc2ccc(-c3cc(C(F)(F)F)nn3C)c(O)c2-c2ccc(Cl)c(C(F)(F)F)c2)cc1] |
| NUCC-0226304 | 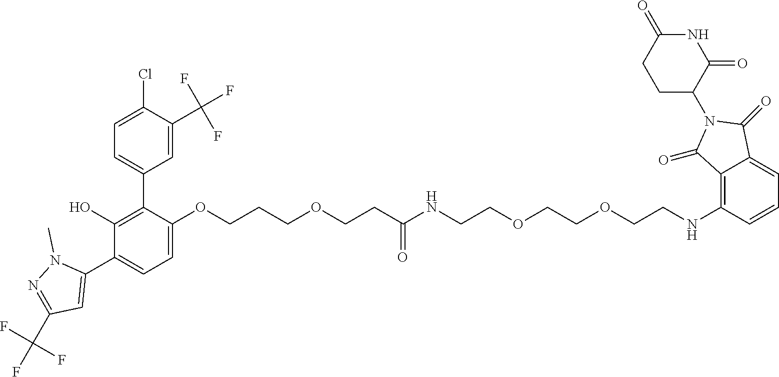 [SMILES: Cn1nc(C(F)(F)F)cc1-c1ccc(OCCCOCCC(=O)NCCOCCOCCNc2cccc3c2C(=O)N(C2CCC(=O)NC2=O)C3=O)c(-c2ccc(Cl)c(C(F)(F)F)c2)c1O] |
| NUCC-0226303 | 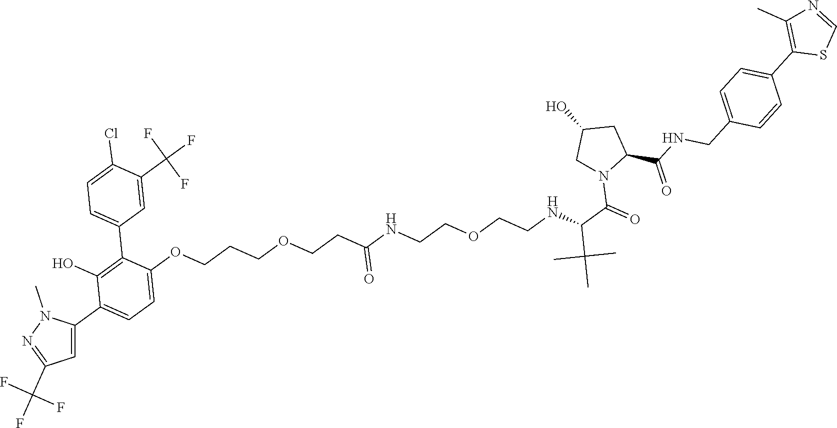 [SMILES: Cc1ncsc1-c1ccc(CNC(=O)[C@@H]2C[C@@H](O)CN2C(=O)[C@@H](NCCOCCNC(=O)CCOCCCOc2ccc(-c3cc(C(F)(F)F)nn3C)c(O)c2-c2ccc(Cl)c(C(F)(F)F)c2)C(C)(C)C)cc1] |
| NUCC-0226302 | 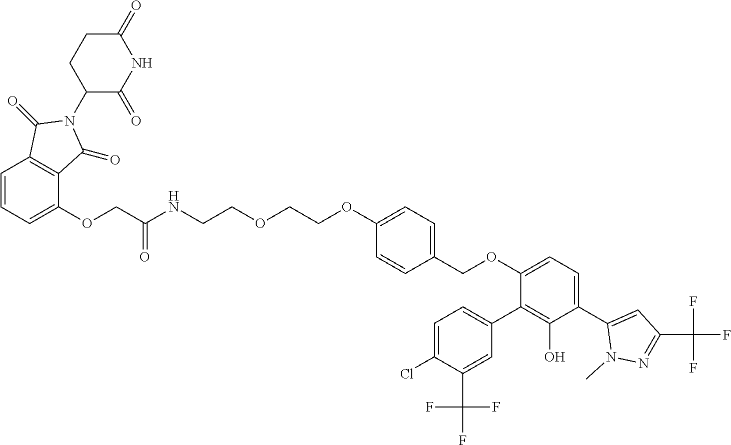 [SMILES: Cn1nc(C(F)(F)F)cc1-c1ccc(OCc2ccc(OCCOCCNC(=O)COc3cccc4c3C(=O)N(C3CCC(=O)NC3=O)C4=O)cc2)c(-c2ccc(Cl)c(C(F)(F)F)c2)c1O] |
| NUCC-0226301 | 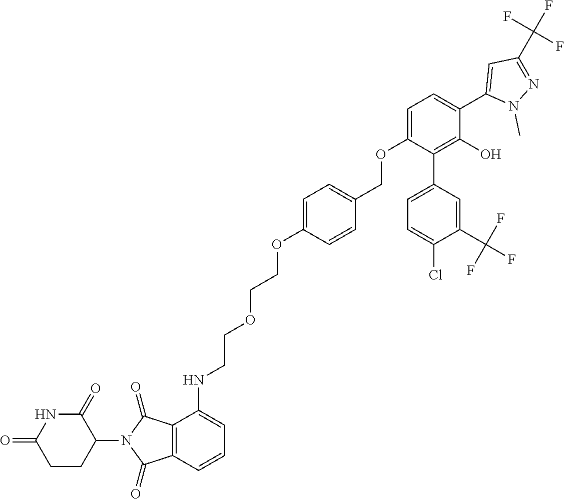 [SMILES: Cn1nc(C(F)(F)F)cc1-c1ccc(OCc2ccc(OCCOCCNc3cccc4c3C(=O)N(C3CCC(=O)NC3=O)C4=O)cc2)c(-c2ccc(Cl)c(C(F)(F)F)c2)c1O] |
| NUCC-0226299 | 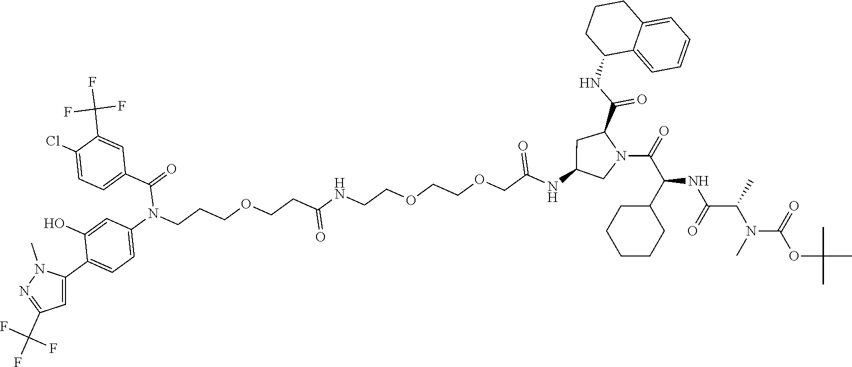 [SMILES: C[C@@H](C(=O)N[C@H](C(=O)N1C[C@@H](NC(=O)COCCOCCNC(=O)CCOCCCN(C(=O)c2ccc(Cl)c(C(F)(F)F)c2)c2ccc(-c3cc(C(F)(F)F)nn3C)c(O)c2)C[C@H]1C(=O)N[C@@H]1CCCc2ccccc21)C1CCCCC1)N(C)C(=O)OC(C)(C)C] |
| NUCC-0226293 | 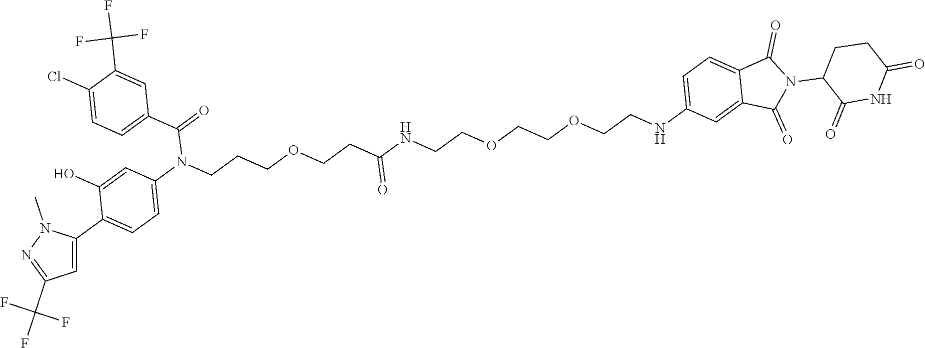 [SMILES: Cn1nc(C(F)(F)F)cc1-c1ccc(N(CCCOCCC(=O)NCCOCCOCCNc2ccc3c(c2)C(=O)N(C2CCC(=O)NC2=O)C3=O)C(=O)c2ccc(Cl)c(C(F)(F)F)c2)cc1O] |
| NUCC-0226292 | 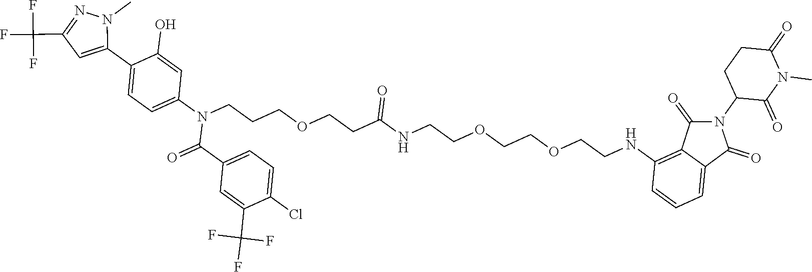 [SMILES: CN1C(=O)CCC(N2C(=O)c3cccc(NCCOCCOCCNC(=O)CCOCCCN(C(=O)c4ccc(Cl)c(C(F)(F)F)c4)c4ccc(-c5cc(C(F)(F)F)nn5C)c(O)c4)c3C2=O)C1=O] |
| NUCC-0226291 | 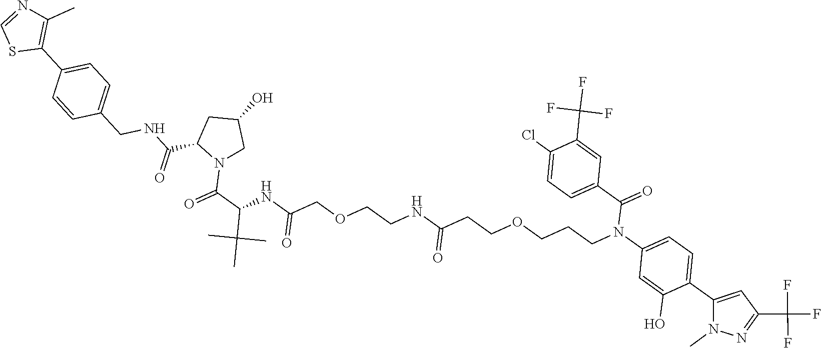 [SMILES: Cc1ncsc1-c1ccc(CNC(=O)[C@@H]2C[C@H](O)CN2C(=O)[C@H](NC(=O)COCCNC(=O)CCOCCCN(C(=O)c2ccc(Cl)c(C(F)(F)F)c2)c2ccc(-c3cc(C(F)(F)F)nn3C)c(O)c2)C(C)(C)C)cc1] |
| NUCC-0226290 | 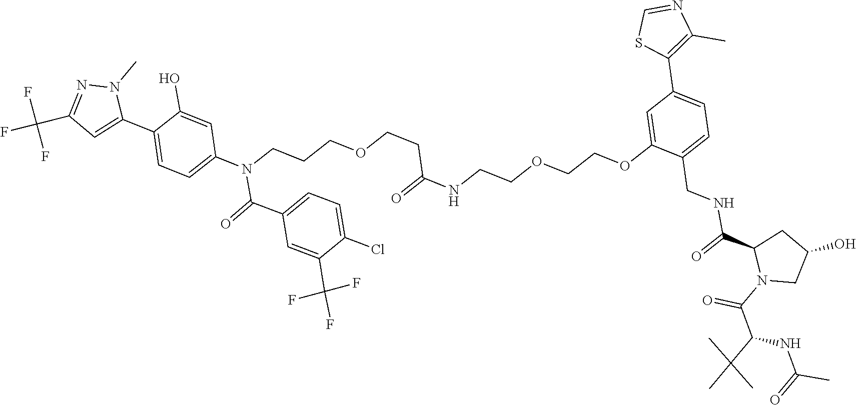 [SMILES: CC(=O)N[C@@H](C(=O)N1C[C@@H](O)C[C@@H]1C(=O)NCc1ccc(-c2scnc2C)cc1OCCOCCNC(=O)CCOCCCN(C(=O)c1ccc(Cl)c(C(F)(F)F)c1)c1ccc(-c2cc(C(F)(F)F)nn2C)c(O)c1)C(C)(C)C] |
| NUCC-0226289 | 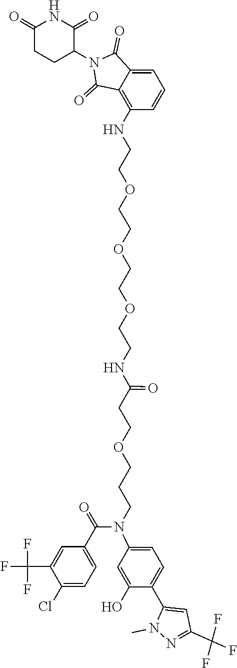 [SMILES: Cn1nc(C(F)(F)F)cc1-c1ccc(N(CCCOCCC(=O)NCCOCCOCCOCCNc2cccc3c2C(=O)N(C2CCC(=O)NC2=O)C3=O)C(=O)c2ccc(Cl)c(C(F)(F)F)c2)cc1O] |
| NUCC-0226288 | 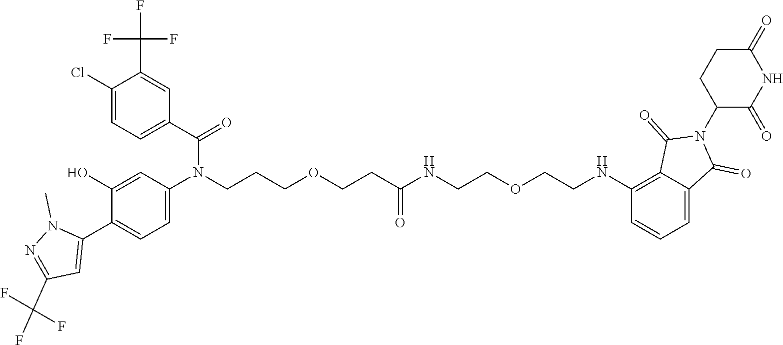 [SMILES: Cn1nc(C(F)(F)F)cc1-c1ccc(N(CCCOCCC(=O)NCCOCCNc2cccc3c2C(=O)N(C2CCC(=O)NC2=O)C3=O)C(=O)c2ccc(Cl)c(C(F)(F)F)c2)cc1O] |
| NUCC-0226280 | 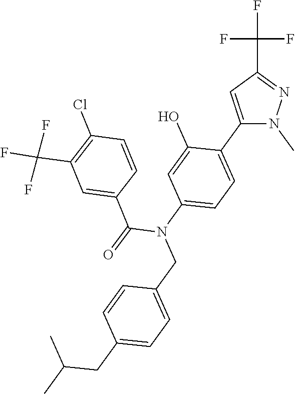 [SMILES: CC(C)Cc1ccc(CN(C(=O)c2ccc(Cl)c(C(F)(F)F)c2)c2ccc(-c3cc(C(F)(F)F)nn3C)c(O)c2)cc1] |
| NUCC-0226279 | 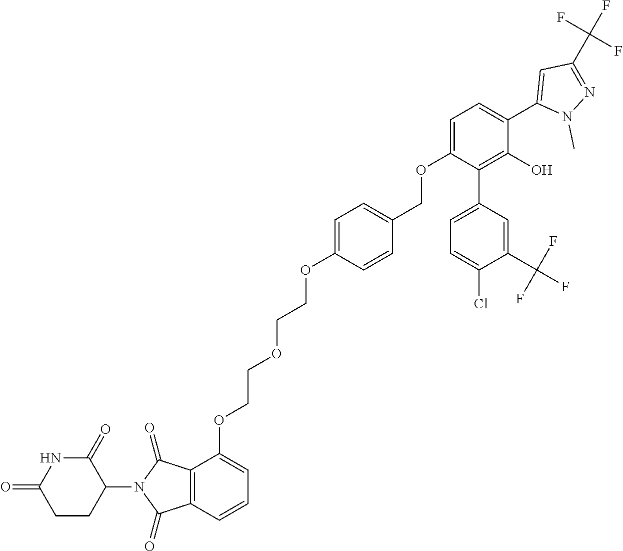 [SMILES: Cn1nc(C(F)(F)F)cc1-c1ccc(OCc2ccc(OCCOCCOc3cccc4c3C(=O)N(C3CCC(=O)NC3=O)C4=O)cc2)c(-c2ccc(Cl)c(C(F)(F)F)c2)c1O] |
| NUCC-0226278 | 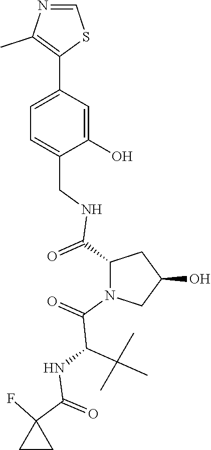 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1] |
| NUCC-0226277 | 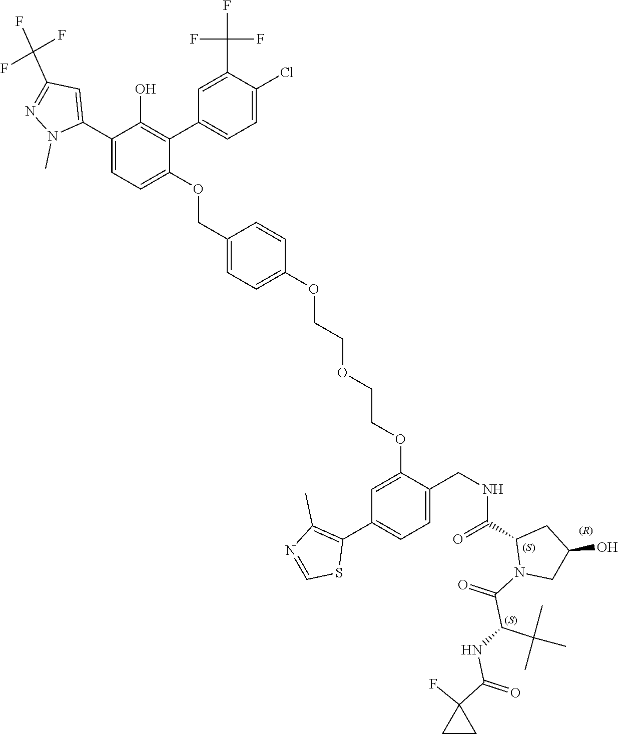 |
| NUCC-0226276 | 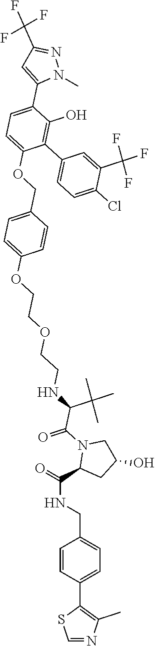 [SMILES: Cc1ncsc1-c1ccc(CNC(=O)[C@@H]2C[C@@H](O)CN2C(=O)[C@@H](NCCOCCOc2ccc(COc3ccc(-c4cc(C(F)(F)F)nn4C)c(O)c3-c3ccc(Cl)c(C(F)(F)F)c3)cc2)C(C)(C)C)cc1] |
| NUCC-0226268 | 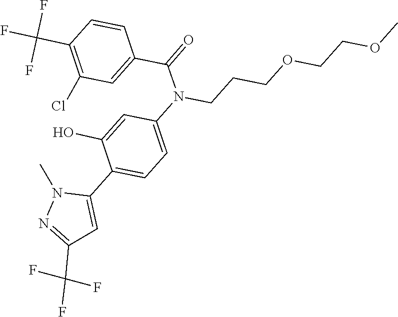 [SMILES: COCCOCCCN(C(=O)c1ccc(C(F)(F)F)c(Cl)c1)c1ccc(-c2cc(C(F)(F)F)nn2C)c(O)c1] |
| NUCC-0226267 | 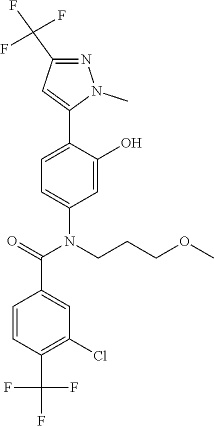 [SMILES: COCCCN(C(=O)c1ccc(C(F)(F)F)c(Cl)c1)c1ccc(-c2cc(C(F)(F)F)nn2C)c(O)c1] |
| NUCC-0226266 | 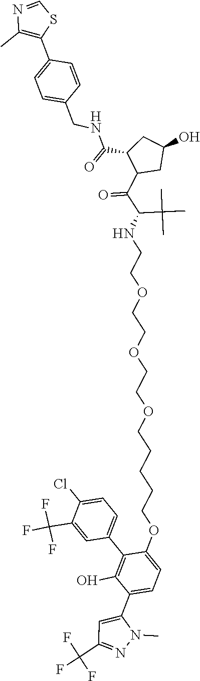 [SMILES: Cc1ncsc1-c1ccc(CNC(=O)[C@@H]2C[C@@H](O)CC2C(=O)[C@@H](NCCOCCOCCOCCCCCOc2ccc(-c3cc(C(F)(F)F)nn3C)c(O)c2-c2ccc(Cl)c(C(F)(F)F)c2)C(C)(C)C)cc1] |
| NUCC-0226265 | 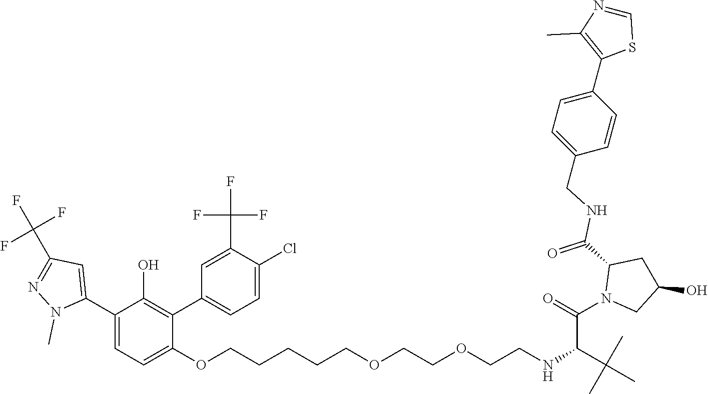 [SMILES: Cc1ncsc1-c1ccc(CNC(=O)[C@@H]2C[C@@H](O)CN2C(=O)[C@@H](NCCOCCOCCCCCOc2ccc(-c3cc(C(F)(F)F)nn3C)c(O)c2-c2ccc(Cl)c(C(F)(F)F)c2)C(C)(C)C)cc1] |
| NUCC-0226264 | 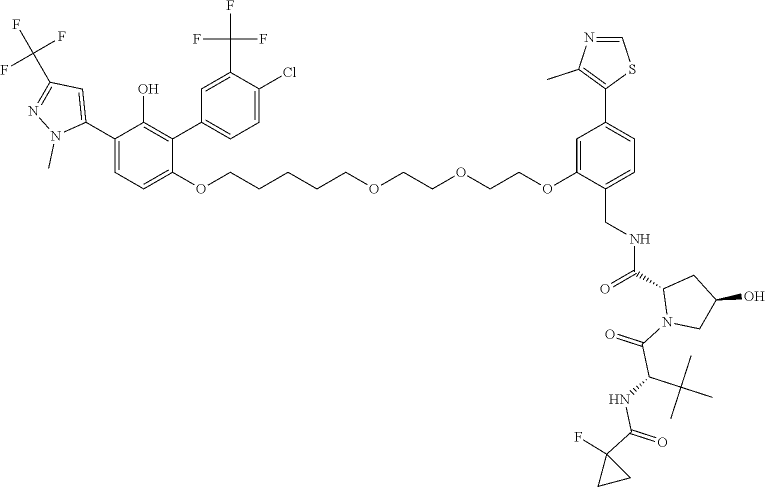 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCCCCOc2ccc(-c3cc(C(F)(F)F)nn3C)c(O)c2-c2ccc(Cl)c(C(F)(F)F)c2)c1] |
| NUCC-0226263 | 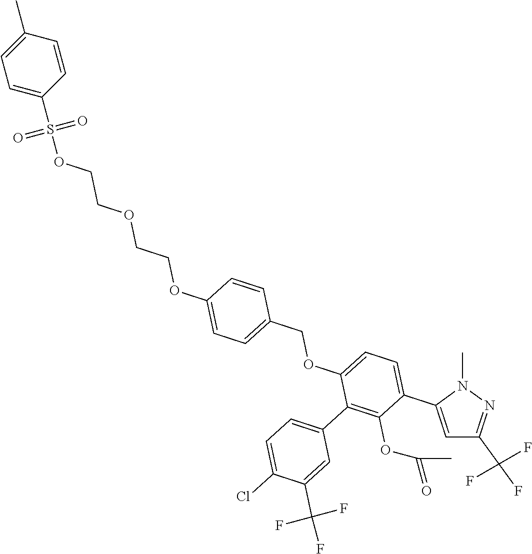 [SMILES: CC(=O)Oc1c(-c2cc(C(F)(F)F)nn2C)ccc(OCc2ccc(OCCOCCOS(=O)(=O)c3ccc(C)cc3)cc2)c1-c1ccc(Cl)c(C(F)(F)F)c1] |
| NUCC-0226262 | 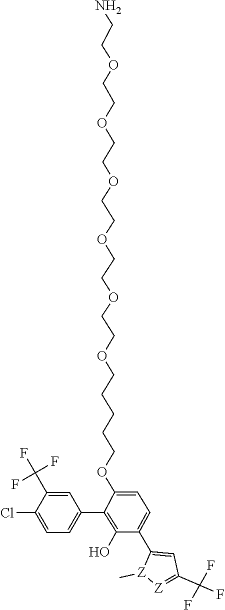 |
| NUCC-0226261 | 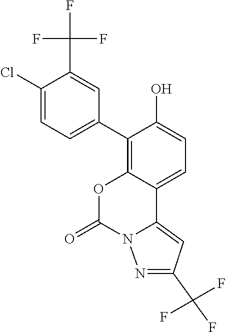 [SMILES: O=c1oc2c(-c3ccc(Cl)c(C(F)(F)F)c3)c(O)ccc2c2cc(C(F)(F)F)nn12] |
| NUCC-0226260 | 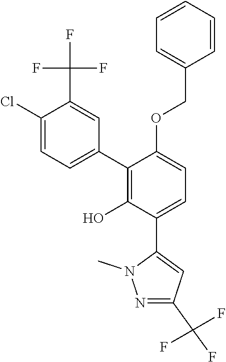 [SMILES: Cn1nc(C(F)(F)F)cc1-c1ccc(OCc2ccccc2)c(-c2ccc(Cl)c(C(F)(F)F)c2)c1O] |
| NUCC-0226259 | 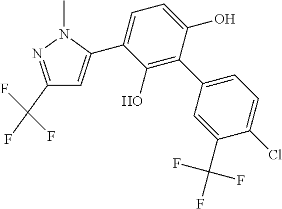 [SMILES: Cn1nc(C(F)(F)F)cc1-c1ccc(O)c(-c2ccc(Cl)c(C(F)(F)F)c2)c1O] |
| NUCC-0226258 | 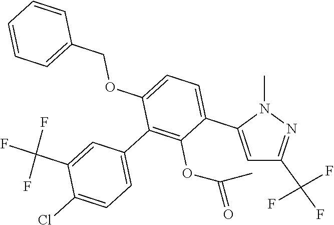 [SMILES: CC(=O)Oc1c(-c2cc(C(F)(F)F)nn2C)ccc(OCc2ccccc2)c1-c1ccc(Cl)c(C(F)(F)F)c1] |
| NUCC-0226257 | 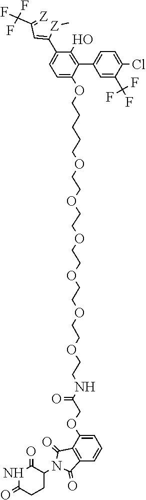 |
| NUCC-0226256 | 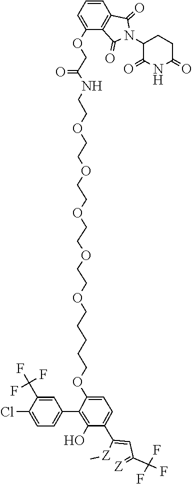 |
| NUCC-0226255 | 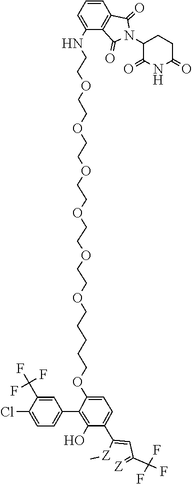 |
| NUCC-0226254 | 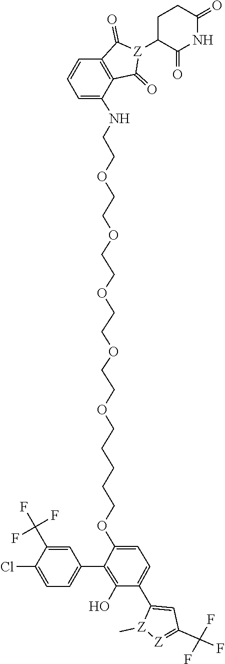 |
| NUCC-0226231 | 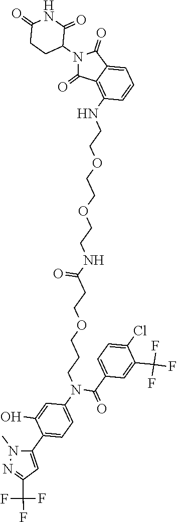 [SMILES: Cn1nc(C(F)(F)F)cc1-c1ccc(N(CCCOCCC(=O)NCCOCCOCCNc2cccc3c2C(=O)N(C2CCC(=O)NC2=O)C3=O)C(=O)c2ccc(Cl)c(C(F)(F)F)c2)cc1O] |
| NUCC-0226230 | 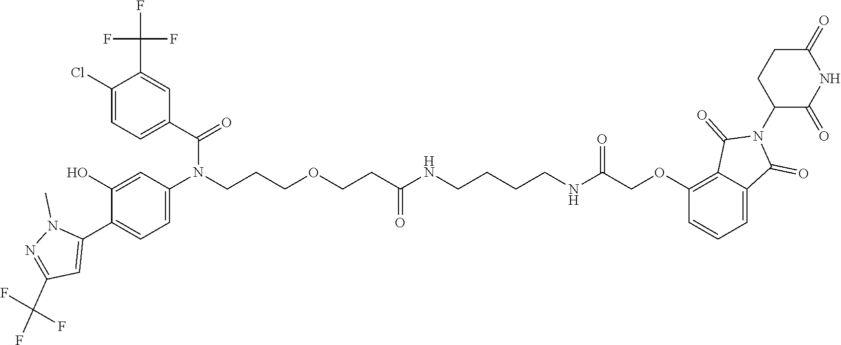 [SMILES: Cn1nc(C(F)(F)F)cc1-c1ccc(N(CCCOCCC(=O)NCCCCNC(=O)COc2cccc3c2C(=O)N(C2CCC(=O)NC2=O)C3=O)C(=O)c2ccc(Cl)c(C(F)(F)F)c2)cc1O] |
| NUCC-0226229 | 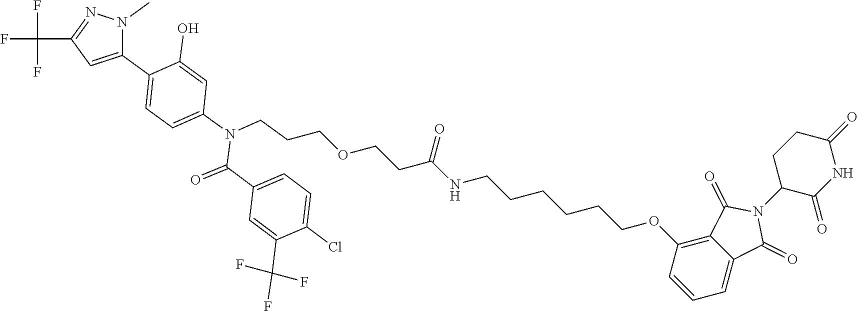 [SMILES: Cn1nc(C(F)(F)F)cc1-c1ccc(N(CCCOCCC(=O)NCCCCCCOc2cccc3c2C(=O)N(C2CCC(=O)NC2=O)C3=O)C(=O)c2ccc(Cl)c(C(F)(F)F)c2)cc1O] |
| NUCC-0226228 | 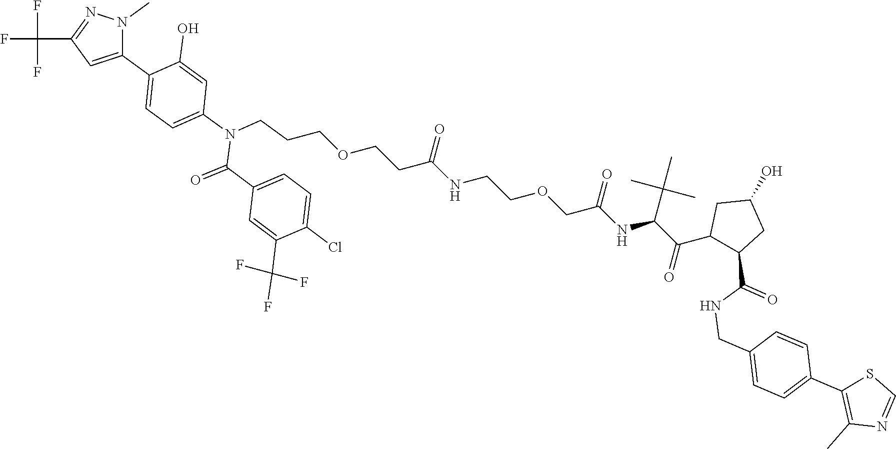 [SMILES: Cc1ncsc1-c1ccc(CNC(=O)[C@@H]2C[C@@H](O)CC2C(=O)[C@@H](NC(=O)COCCNC(=O)CCOCCCN(C(=O)c2ccc(Cl)c(C(F)(F)F)c2)c2ccc(-c3cc(C(F)(F)F)nn3C)c(O)c2)C(C)(C)C)cc1] |
| NUCC-0226227 | 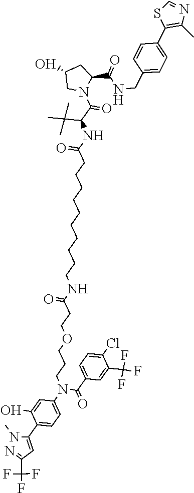 |
| NUCC-226226 | 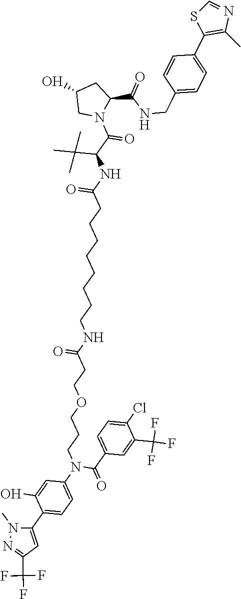 |
| NUCC-0226225 | 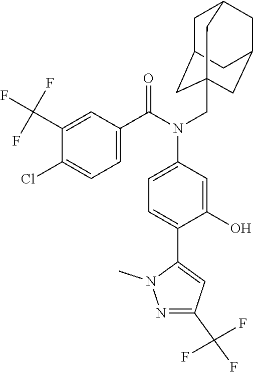 [SMILES: Cn1nc(C(F)(F)F)cc1-c1ccc(N(CC23CC4CC(CC(C4)C2)C3)C(=O)c2ccc(Cl)c(C(F)(F)F)c2)cc1O] |
| NUCC-0226224 | 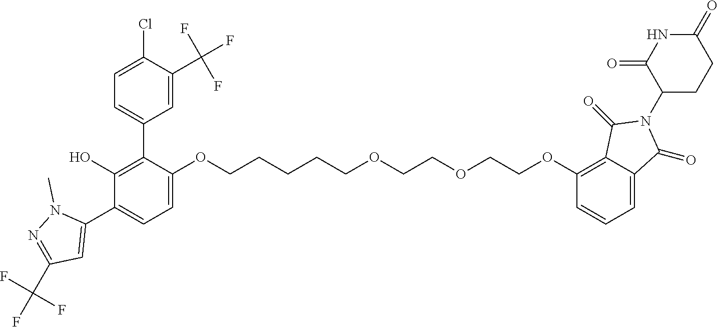 [SMILES: Cn1nc(C(F)(F)F)cc1-c1ccc(OCCCCCOCCOCCOc2cccc3c2C(=O)N(C2CCC(=O)NC2=O)C3=O)c(-c2ccc(Cl)c(C(F)(F)F)c2)c1O] |
| NUCC- 0226223 | 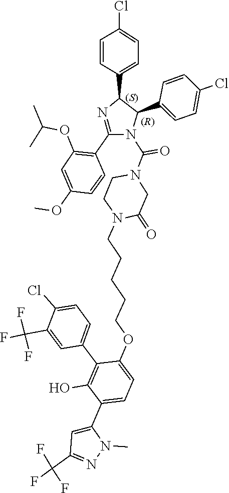 |
| NUCC-0226221 | 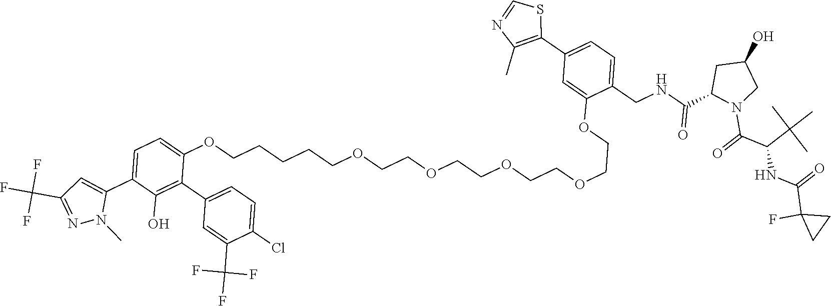 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCCCCOc2ccc(-c3cc(C(F)(F)F)nn3C)c(O)c2-c2ccc(Cl)c(C(F)(F)F)c2)c1] |
| NUCC-0226220 | 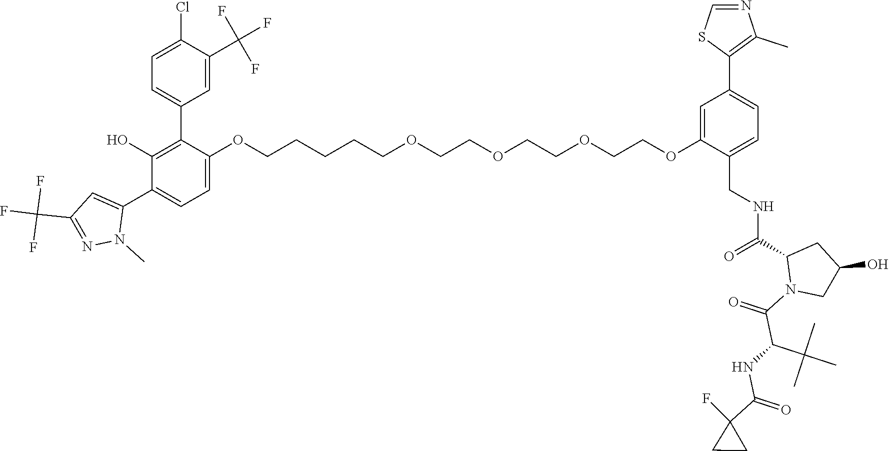 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCCCCOc2ccc(-c3cc(C(F)(F)F)nn3C)c(O)c2-c2ccc(Cl)c(C(F)(F)F)c2)c1] |
| NUCC-0226219 | 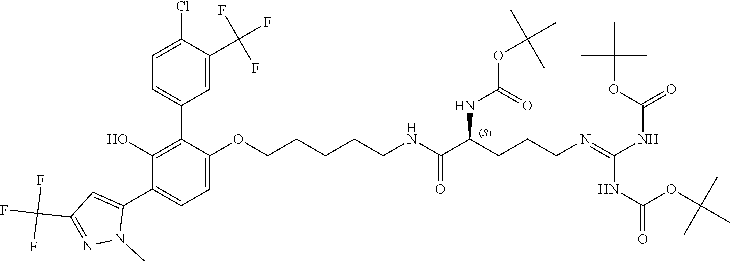 |
| NUCC-0227169 | 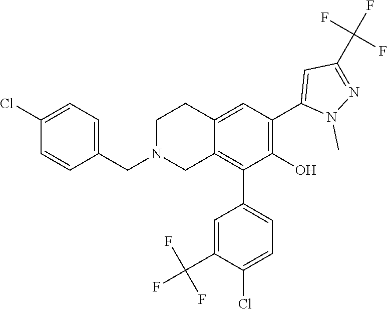 [SMILES: Cn1nc(C(F)(F)F)cc1-c1cc2c(c(-c3ccc(Cl)c(C(F)(F)F)c3)c1O)CN(Cc1ccc(Cl)cc1)CC2] |
| NUCC-0227173 | 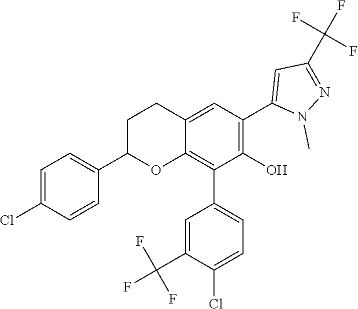 [SMILES: Cn1nc(C(F)(F)F)cc1-c1cc2c(c(-c3ccc(Cl)c(C(F)(F)F)c3)c1O)OC(c1ccc(Cl)cc1)CC2] |
| NUCC-0227174 | 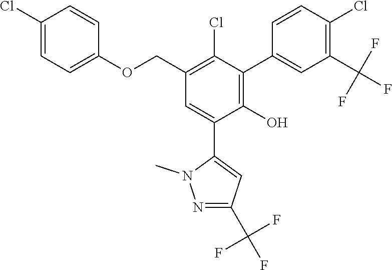 [SMILES: Cn1nc(C(F)(F)F)cc1-c1cc(COc2ccc(Cl)cc2)c(Cl)c(-c2ccc(Cl)c(C(F)(F)F)c2)c1O] |
| NUCC-0202416 | 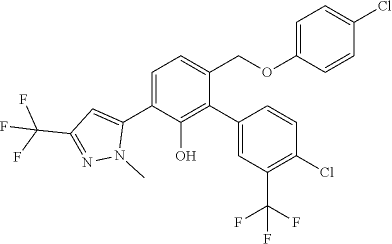 [SMILES: Cn1nc(C(F)(F)F)cc1-c1ccc(COc2ccc(Cl)cc2)c(-c2ccc(Cl)c(C(F)(F)F)c2)c1O] |
| NUCC-0227298 | 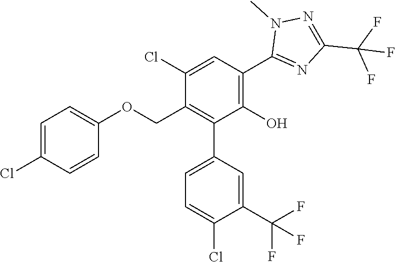 [SMILES: Cn1nc(C(F)(F)F)nc1-c1cc(Cl)c(COc2ccc(Cl)cc2)c(-c2ccc(Cl)c(C(F)(F)F)c2)c1O] |
| NUCC-0227263 | 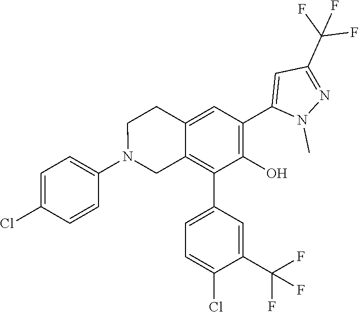 [SMILES: Cn1nc(C(F)(F)F)cc1-c1cc2c(c(-c3ccc(Cl)c(C(F)(F)F)c3)c1O)CN(c1ccc(Cl)cc1)CC2] |
| NUCC-0227247 | 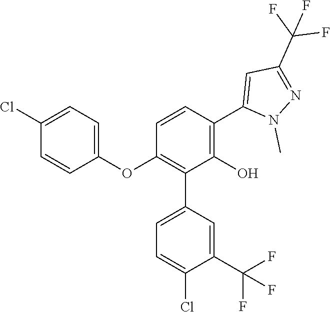 [SMILES: Cn1nc(C(F)(F)F)cc1-c1ccc(Oc2ccc(Cl)cc2)c(-c2ccc(Cl)c(C(F)(F)F)c2)c1O] |
| NUCC-0227245 | 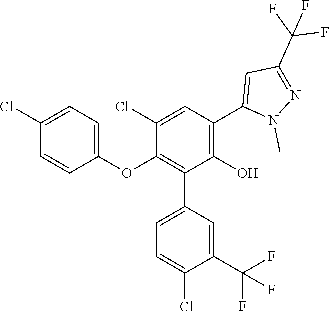 [SMILES: Cn1nc(C(F)(F)F)cc1-c1cc(Cl)c(Oc2ccc(Cl)cc2)c(-c2ccc(Cl)c(C(F)(F)F)c2)c1O] |
| NUCC-0227246 | 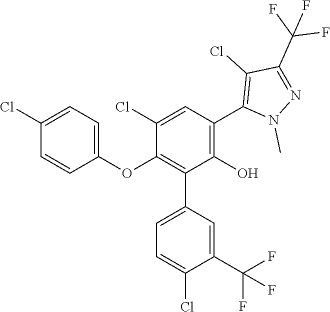 [SMILES: Cn1nc(C(F)(F)F)c(Cl)c1-c1cc(Cl)c(Oc2ccc(Cl)cc2)c(-c2ccc(Cl)c(C(F)(F)F)c2)c1O] |
| NUCC-0227244 | 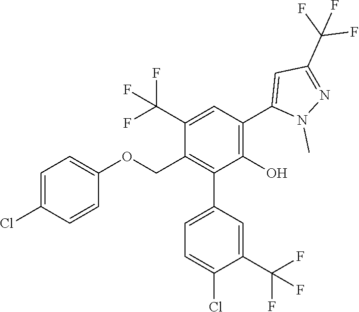 [SMILES: Cn1nc(C(F)(F)F)cc1-c1cc(C(F)(F)F)c(COc2ccc(Cl)cc2)c(-c2ccc(Cl)c(C(F)(F)F)c2)c1O] |

Table 2. Biological data of selected compounds. For IC50 determination. PC3 and PC12 cells were treated with increasing concentrations of the indicated compounds for 72 hrs and cell viability assessed using CellTitreGlo Assay (Promega). For MYC and ATF4 protein level determination. PC3 cells were treated with the indicated compounds for 24 hr sand cell lysates processed for western blot with MYC and ATF4 antibodies using Actin as a control. Quantification analyses were performed by Biorad ChemiDoc Imager and Bio-Rad Image Lab software.

| | | | Relative MYC | Relative ATF4 |
|---|---|---|---|---|
| | PC3 | PC12 | protein at | protein at 10 μM |
| | IC50 | IC50 | 10 μM (%, | (fold change, |
| Molecule Name | (μM) | (μM) | PC3 24 h) | PC3 24 h) |
| NUCC-0227066 | 8.53 | | 84.2 | 3.9 |
| NUCC-0227065 | 0.74 | 3.8 | 35.4 | 13.4 |
| NUCC-0227064 | 0.399 | 8.273 | 42.04 | 12.66 |
| NUCC-0227063 | 0.985 | 5.018 | 37.17 | 12.36 |
| NUCC-0227062 | 0.964 | 2.313 | 40.7 | 15 |
| NUCC-0227038 | 1.615 | 15.29 | 34.45 | 15.1 |
| NUCC-0227037 | 0.96 | 11.06 | 33.59 | 15.07 |
| NUCC-0226915 | 14.2 | 10.49 | 86.9 | 0.95 |
| NUCC-0226914 | 1.013 | 3.5 | 48.5 | 14.1 |
| NUCC-0226661 | | | | |
| NUCC-0226660 | >50 | 9.22 | 94.4 | 2.5 |
| NUCC-0226659 | | | 90.3 | 1.5 |
| NUCC-0226658 | | | | |
| NUCC-0226657 | 7.51 | | 56.6 | 7.7 |
| NUCC-0226656 | | | 103 | 1.5 |
| NUCC-0226652 | 4.42 | 16.6 | 72.3 | 5.2 |
| NUCC-0226606 | 2.23 | 16.65 | 38.2 ± 3.9 | 9.7 ± 1.2 |
| NUCC-0226605 | 1 | 10.46 | 34.7 ± 3.2 | 10.3 ± 1.5 |
| NUCC-0226604 | | | | |
| NUCC-0226603 | | | | |
| NUCC-0226597 | | | | |
| NUCC-0226596 | | | | |
| NUCC-0226595 | 0.74 | 4.03 | 69.8 | 6.3 |
| NUCC-0226574 | 3.59 | | 43.7 | 12 |
| NUCC-0226566 | 3.88 | | 43.7 | 10.5 |
| NUCC-0226548 | N/A | | 19.5 | 4.7 |
| NUCC-0226545 | 2.2 | 10.8 | 19.5 | 4.7 |
| NUCC-0226530 | 12 | 9.4 | 35.5 | 3.0 |
| NUCC-0226529 | 3.1 | 19.4 | 41.3 | 2.4 |
| NUCC-0226528 | 4.5 | 30.5 | 19.8 | 2.0 |
| NUCC-0226527 | 6.2 | | 50.8 | 1.2 |
| NUCC-0226520 | 12.7 | | 67.5 | 1.1 |
| NUCC-0226504 | 7.3 | | 32.5 | 5.2 |
| NUCC-0226503 | | | 128.6 | 3.0 |
| NUCC-0226502 | 14.1 | | 121.6 | 1.4 |
| NUCC-0226501 | 4.6 | | 59.4 | 9.3 |
| NUCC-0226500 | 1.6 | 8.6 | 72.4 | 7.7 |
| NUCC-0226499 | 2.4 | 13.5 | 37.3 | 17.1 |
| NUCC-0226498 | 40.9 | | 67.8 | 4.8 |
| NUCC-0226497 | 24.8 | | 93.0 | 5.9 |
| NUCC-0226496 | 5.3 | | 25.4 | 10.3 |
| NUCC-0226495 | 3.8 | 597 | 15.6 | 11.5 |
| NUCC-0226438 | 5.3 | | 52.3 | 3.7 |
| NUCC-0226437 | 5.4 | | 50.7 | 9.0 |
| NUCC-0226436 | 3.4 | | 30.3 | 11.8 |
| NUCC-0226435 | 5.4 | | 47.4 | 4.7 |
| NUCC-0226434 | 4.3 | | 20.0 | 9.4 |
| NUCC-0226433 | 4.0 | | 35.0 | 10.6 |
| NUCC-0226410 | | | | |
| NUCC-0226305 | 8.4 | | 52.4 | 2.9 |
| NUCC-0226304 | | | 169.5 | 4.6 |
| NUCC-0226303 | 10.9 | | 24.0 | 1.6 |
| NUCC-0226302 | N/A | | 79.4 | 2.0 |
| NUCC-0226301 | 1.1 | 3.8 | 48.5 | 3.2 |
| NUCC-0226299 | 3.8 | 11.3 | 42.1 | 5.4 |
| NUCC-0226293 | 4.7 | | 18.4 | 3.6 |
| NUCC-0226292 | 10.1 | | 65.1 | 1.1 |
| NUCC-0226291 | 4.0 | 14.6 | 54.6 | 1.8 |
| NUCC-0226290 | 5.1 | | 27.4 | 2.9 |
| NUCC-0226289 | 7.4 | | 119.6 | 7.0 |
| NUCC-0226288 | 6.0 | | 54.2 | 6.6 |
| NUCC-0226280 | | | 45.2 | 10.3 |
| NUCC-0226279 | | | 85.1 | 2.1 |
| NUCC-0226278 | | | | |
| NUCC-0226277 | | | 97.6 | 1.6 |
| NUCC-0226276 | | | 70.0 | 0.4 |
| NUCC-0226268 | | | 52.9 | 5.4 |
| NUCC-0226267 | 4.6 | | 46.4 | 8.0 |
| NUCC-0226266 | | | 91.8 | 1.7 |
| NUCC-0226265 | | | 115.4 | 2.2 |
| NUCC-0226264 | | | 102.5 | 1.3 |
| NUCC-0226263 | | | 113.0 | 0.7 |
| NUCC-0226262 | 3.5 | 10.9 | 163.6 | 3.6 |
| NUCC-0226261 | | | 93.1 | 4.4 |
| NUCC-0226260 | | | 41.5 | 4.6 |
| NUCC-0226259 | | | 52.0 | 3.3 |
| NUCC-0226258 | | | 56.4 | 5.8 |
| NUCC-0226257 | 16.1 | | 75.3 | 4.7 |
| NUCC-0226256 | 19.7 | | 77.1 | 3.4 |
| NUCC-0226255 | 8.0 | | 71.3 | 2.9 |
| NUCC-0226254 | 12.8 | | 77.4 | 2.9 |
| NUCC-0226231 | 8.5 | | 53.6 | 14.3 |
| NUCC-0226230 | N/A | | 105.0 | 1.9 |
| NUCC-0226229 | 15.6 | | 56.4 | 16.4 |
| NUCC-0226228 | 6.0 | | 38.1 | 23.1 |
| NUCC-0226227 | 22.3 | | 101.6 | 10.2 |
| NUCC-0226226 | 5.1 | | 81.9 | 10.6 |
| NUCC-0226225 | 2.1 | 14.5 | 28.4 | 15.2 |
| NUCC-0226224 | 10.1 | | 84.2 | 8.1 |
| NUCC-0226223 | 18.6 | | 91.5 | 5.5 |
| NUCC-0226221 | | | 85.9 | |
| NUCC-0226220 | | | 90.1 | |
| NUCC-0226219 | | | 90.6 | |
| NUCC-0227169 | 3.28 | 8.01 | 41.78 | 14.57 |
| NUCC-0227173 | 4.74 | 14.14 | 62.91 | 9 |
| NUCC-0227174 | | | | |
| NUCC-0202416 | 4.71 | inactive | 38.9 | 9.3 |
| NUCC-0227244 | 0.57 | 4.55 | 39.5 | 22.8 |
| NUCC-0227245 | 0.49 | 3.81 | 37.1 ± 1.0 | 20.3 ± 3.7 |
| NUCC-0227246 | 0.91 | 5.78 | 37.3 | 23.5 |
| NUCC-0227247 | 1.14 | 7.12 | 46.7 | 18.3 |
| MYCi975 | 2.9 ± 0.5 | >16 | | |

The structure of “MYCi975” is shown below:

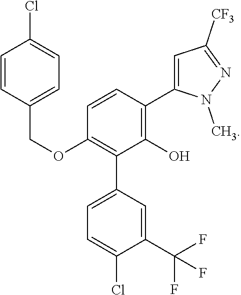 [SMILES: Cn1nc(C(F)(F)F)cc1-c1ccc(OCc2ccc(Cl)cc2)c(-c2ccc(Cl)c(C(F)(F)F)c2)c1O]

Table 3. Biological data of selected compounds. Male FVB/N mice were inoculated with 1 million MycCaP;Pten-KO cells subcutaneously in their flanks. Once tumors are established (˜200 mm3), mice were treated with the indicated compounds at 100 mk/kg/d p.o. and tumor volume measured using calipers. Tumor growth inhibition (TGI) was determined after 14 d. Toxicity was assessed as −, 0-5% weight loss; +, 5-15% weight loss; ++, 15-20% weight loss; +++, >20% weight loss or death. PK: Plasma levels of compounds were determined after 1 mg/kg dosing PO in female BALB/C mice. 1, T½˜4-6 hr; 2, T½ 8-12 h; 3, T½>20 hr.

| | PK | Efficacy MycCaP; | |
|---|---|---|---|
| | Relative | Pten-ko (TGI @ | Toxicity @ |
| | to 975 | 100 mg/kg/d po) | 100 mg/kg/d |
| MYCi975 | 1 | 50 | − |
| NUCC-0226545 | 1 | | |
| NUCC-0226605 | 2 | 90 | ++ |
| NUCC-0226606 | 3 | 75 | − |
| NUCC-0227037 | 3 | 80 | ++ |
| NUCC-0202416 | 3 | | |
| NUCC-0227244 | 3 | | ++ |
| NUCC-0227245 | 2 | | +++ |
| NUCC-0227246 | 2 | | |
| NUCC-0227247 | 2 | | |

Table 4. Plasma Pharmacokinetic parameters 4 compounds after PO (1 mg/kg) administration to Female C57BL/6 mice for compound NUCC-0226545. Plasma levels of compounds were determined after 1 mg/kg dosing PO in female BALB/C mice.

| | T_{1/2}_{_{—}}range* | T_{1/2} | Tₘₐₓ | Cₘₐₓ | AUC₀₋ₜ |
|---|---|---|---|---|---|
| Compound | (h) | (h) | (h) | (ng/mL) | (ng · h/mL) |
| NUCC- | 8-24 | 4.26 | 4 | 464 | 4423 |
| 0200975 | | | | | |
| NUCC- | 8-24 | 4.30 | 4 | 472 | 4761 |
| 0226545 | | | | | |

| | AUC_{0-∞} | AUMC₀₋ₜ | AUMC_{0-∞} | MRT_{INF} |
|---|---|---|---|---|
| | (ng · h/mL) | (h*h*ng/mL) | (h*h*ng/mL) | (h) |
| NUCC- | | 34444 | | 7.82 |
| 0200975 | | | | |
| NUCC- | | 36107 | | 7.57 |
| 0226545 | | | | |

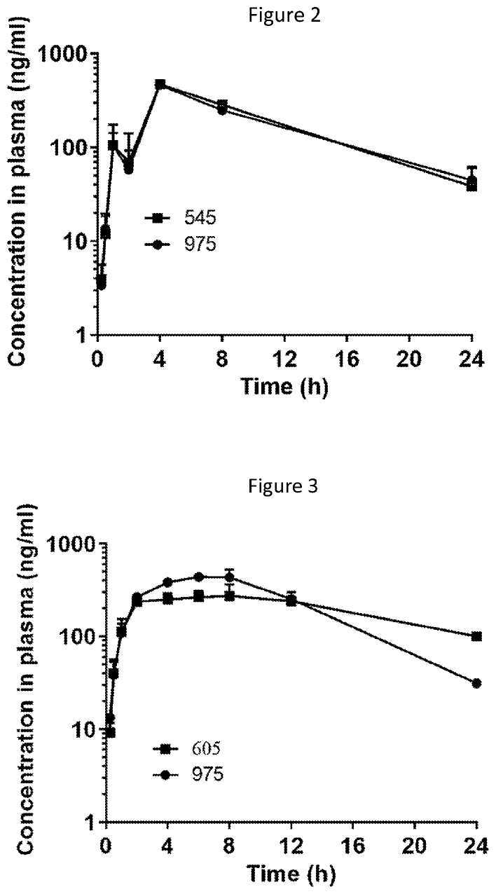

For PK study of NUCC-0226545 by P.O., see FIG. 2.

Table 5. Plasma Pharmacokinetic parameters 4 compounds after PO (1 mg/kg) administration to Female C57BL/6 mice for compounds NUCC-0226605 and NUCC-0226606. Plasma levels of compounds were determined after 1 mg/kg dosing PO in female BALB/C mice.

| | T_{1/2}_{_{—}}range* | T_{1/2} | Tₘₐₓ | Cₘₐₓ | AUC₀₋ₜ |
|---|---|---|---|---|---|
| Compound | (h) | (h) | (h) | (ng/mL) | (ng · h/mL) |
| NUCC- | 8-24 | 4.16 | 6 | 439 | 5646 |
| 0200975 | | | | | |
| NUCC- | | | 8 | 274 | 4829 |
| 0226605 | | | | | |
| NUCC- | | | 12 | 841 | 15194 |
| 0226606 | | | | | |

| | AUC_{0-∞} | AUMC₀₋ₜ | AUMC_{0-∞} | MRT_{INF} |
|---|---|---|---|---|
| | (ng · h/mL) | (h*h*ng/mL) | (h*h*ng/mL) | (h) |
| NUCC- | 5834 | 48358 | 53976 | 9.25 |
| 0200975 | | | | |
| NUCC- | | 49995 | | |
| 0226605 | | | | |
| NUCC- | | 177148 | | |
| 0226606 | | | | |

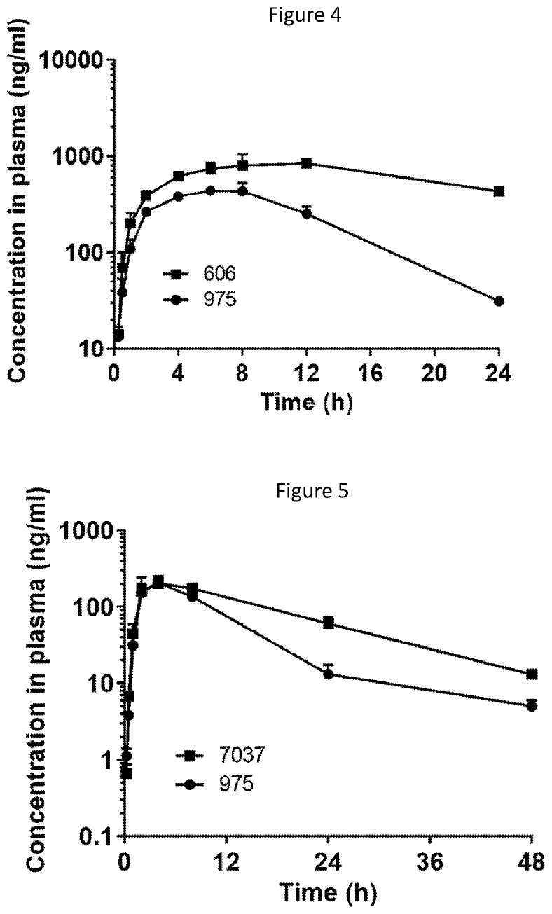

For PK studies of NUCC-0226605 and NUCC-0226606 by P.O., see FIGS. 3 and 4.

Table 6. Plasma Pharmacokinetic parameters 4 compounds after PO (1 mg/kg) administration to Female C57BL/6 mice for compounds NUCC-0227037 and NUCC-0202416. Plasma levels of compounds were determined after 1 mg/kg dosing PO in female BALB/C mice.

| | T_{1/2}_{_{—}}range* | T_{1/2} | Tₘₐₓ | Cₘₐₓ | AUC₀₋ₜ |
|---|---|---|---|---|---|
| Compound | (h) | (h) | (h) | (ng/mL) | (ng · h/mL) |
| NUCC- | 8-48 | 8.77 | 4 | 206 | 2556 |
| 0200975 | | | | | |
| NUCC- | 8-48 | 10.7 | 4 | 202 | 4002 |
| 0227037 | | | | | |
| NUCC- | | | 8 | 244 | 5763 |
| 0202416 | | | | | |

| | AUC_{0-∞} | AUMC₀₋ₜ | AUMC_{0-∞} | MRT_{INF} |
|---|---|---|---|---|
| | (ng · h/mL) | (h*h*ng/mL) | (h*h*ng/mL) | (h) |
| NUCC- | 2620 | 23023 | 26881 | 10.3 |
| 0200975 | | | | |
| NUCC- | 4205 | 53530 | 66373 | 15.8 |
| 0227037 | | | | |
| NUCC- | | 88636 | | |
| 0202416 | | | | |

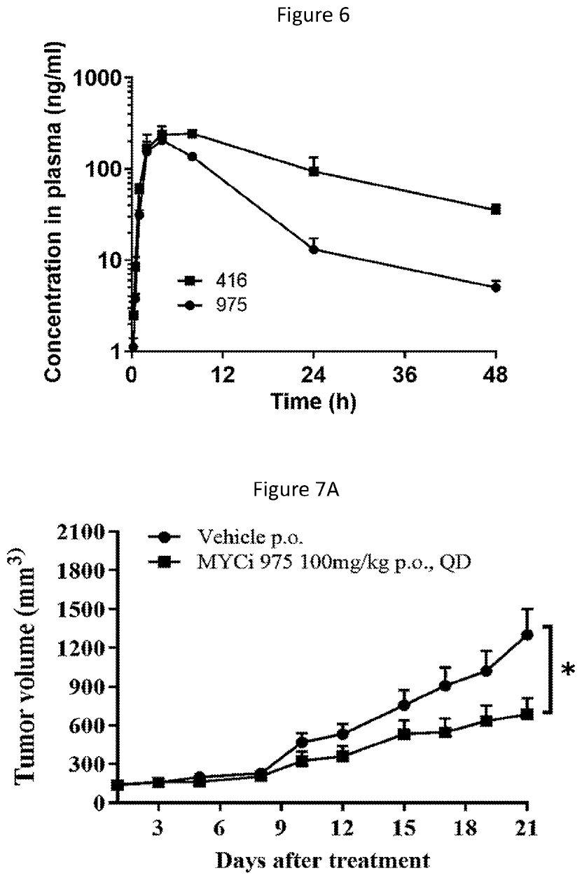

For PK studies of NUCC-0227037 and NUCC-0202416 by P.O., see FIGS. 5 and 6.

Table 7. Tumor Growth Inhibition data. Male FVB/N mice were inoculated with 1 million MycCaP; Pten-KO cells subcutaneously in their flanks. Once tumors are established (˜200 mm3), mice were treated with the indicated compounds at 100 mk/kg/d p.o. and tumor volume measured using calipers. Tumor growth inhibition (TOD was determined after 14 d.

| Compound | Dosage | TGI |
|---|---|---|
| NUCC-0200975 (MYCi 975) | 100 mg/kg p.o., QD | 54.01% |
| NUCC-0226605 (MYCi 605) | 100 mg/kg p.o., QD | 92.51% |
| | 30 mg/kg p.o., QD | 65.67% |
| | 10 mg/kg p.o., QD | 56.29% |
| NUCC-0226606 (MYCi 606) | 100 mg/kg p.o., QD | 74.6% |
| | 30 mg/kg p.o., QD | 56.4% |
| | 10 mg/kg p.o., QD | 43.1% |
| NUCC-0227037 (MYCi 7037) | 100 mg/kg p.o., QD | 81.48% |
| | 30 mg/kg p.o., QD | 57.24% |
| | 10 mg/kg p.o., QD | 43.99% |

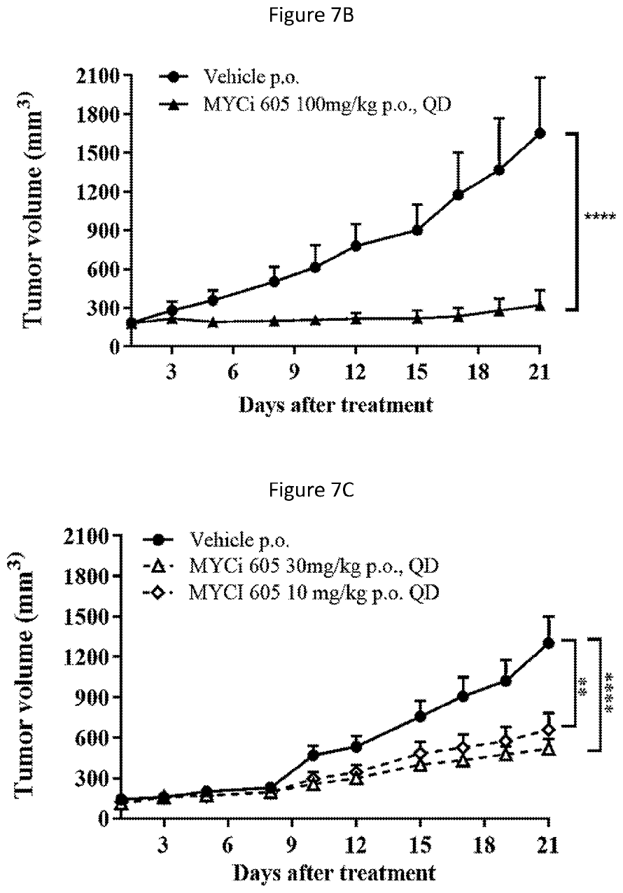

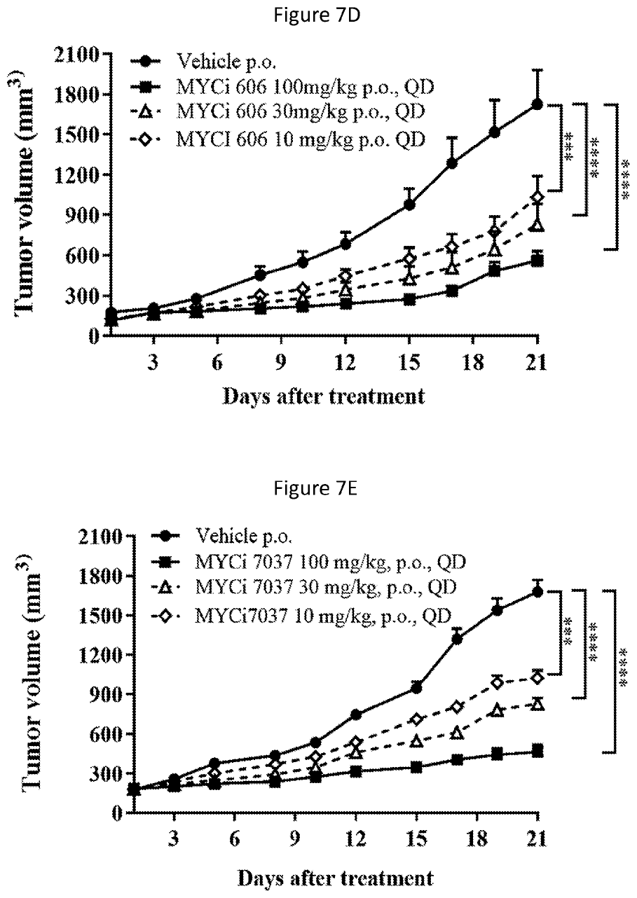

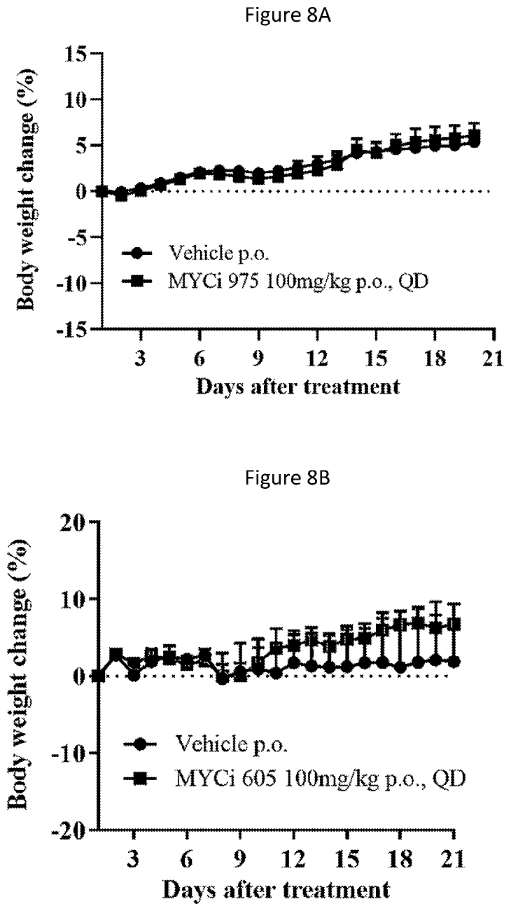

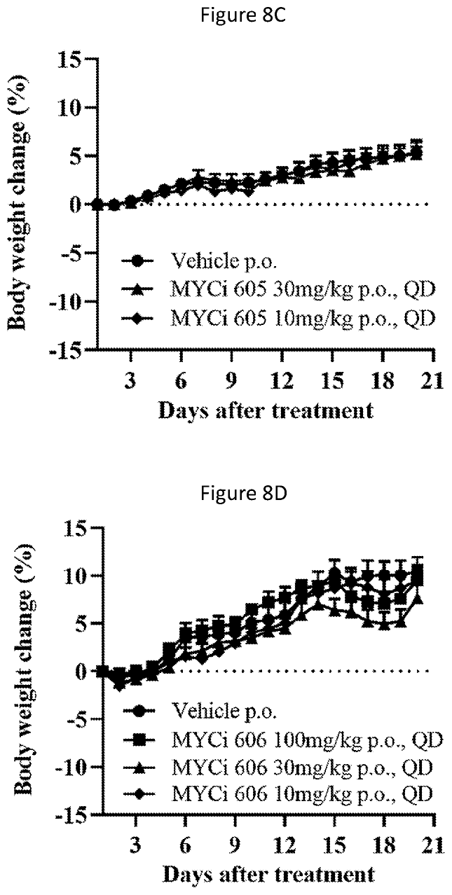

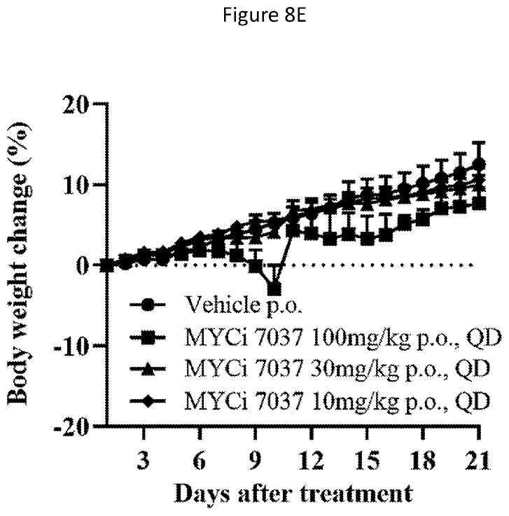

For MyC-CaP PTEN KO/FVB Allograft data for compounds NUCC-0200975. NUCC-0226605. NUCC-0226606, and NUCC-0227037, see FIGS. 7A-7E and 8A-8E

We claim:

1. A compound having a formula I: 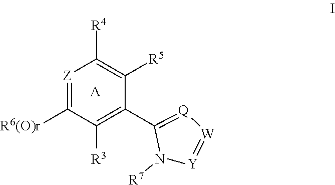
wherein
W is CR⁸ or N;
Y is CH or N;
Q is CR² or N;
Z is C(Alk²)_{q}(X)ₚ(Alk¹)ₙR¹ or N;
R¹ is hydrogen, halo, alkyl, aryl, benzyl, heteroaryl, cycloalkyl, alkoxy, or cycloheteroalkyl, wherein R¹ is optionally substituted at one or more positions with one or more of alkyl, alkoxy, cycloalkyl, haloalkyl, haloalkoxy, aryl, hydroxyl, halo, cyano, amido, carboxyl, —(CH₂)—NH—R⁹, —(((CH₂)₂)ₘ—R¹⁵, or —OR¹¹;
Alk¹ is straight-chain or branched alkylenyl, or a cycloalkylenyl;
n is 0, 1, or 2;
p is 0 or 1;
Alk² is straight-chain or branched alkylenyl;
q is 0 or 1;
r is 0 or 1;
m is an integer selected from 1 to 20;
X is O or NR¹³;
R² is hydrogen or halo;
R³ is alkyl, alkoxy, haloalkyl, haloalkoxy, hydroxyl, —O—C(O)-alkyl, or halo;
R⁴ is hydrogen, halo, amino, alkyl, or haloalkyl; or R⁴ is aryl or benzyl optionally substituted at one or more ring positions with one or more of alkyl, alkoxy, haloalkyl, haloalkoxy, aryl, hydroxyl, halo, cyano, amido, carboxyl, aryloxy, and alkylaryloxy; or R⁴ is alkyl optionally substituted with halo-substituted aryloxy; or R⁴ together with R¹ form a cycloheteroalkyl fused to ring A, wherein the cycloheteroalkyl fused to ring A is optionally substituted with aryl or alkylaryl optionally substituted with halogen;
R⁵ is hydrogen, halo, alkyl, aryl, alkylaryl, heteroaryl, cycloalkyl, or cycloheteroalkyl, optionally R⁵ is substituted at one or more positions with one or more of alkyl, alkoxy, haloalkyl, haloalkoxy, hydroxyl, halo, cyano, carboxyamido, carboxy, aryloxy, and heteroaryloxy;
R⁶ is hydrogen, halo, alkyl, aryl, alkylaryl, heteroaryl, cycloalkyl, or cycloheteroalkyl, optionally R⁶ is substituted at one or more positions with one or more of alkyl, alkoxy, haloalkyl, haloalkoxy, hydroxyl, halo, cyano, carboxyamido, carboxy, aryloxy, and heteroaryloxy;
R⁷ is alkyl;
R⁸ is hydrogen, cyano, amino, alkyl, alkoxy, haloalkyl, haloalkoxy, aryl, benzyl, hydroxyl, halo, amido, and carboxyl;
R⁹ is alkyl optionally substituted with one or more of —P(O)(OR¹)₂;
R¹⁰ is hydrogen or alkyl;
R¹¹ is alkyl optionally substituted with one or more of —P(O)(OR¹²)₂;
R¹² is hydrogen or alkyl;
R¹³ is hydrogen, alkyl, or —C(O)R¹⁴;
R¹⁴ is aryl optionally substituted at one or more ring positions with one or more of alkyl, alkoxy, haloalkyl, haloalkoxy, aryl, hydroxyl, halo, cyano, amido, and carboxyl;
R¹⁵ is OR¹⁶, —OS(O)₂—R¹⁶, or NR¹⁷R¹⁸;
R¹⁶ is alkyl or aryl, wherein R¹⁶ is optionally substituted at one or more positions with one or more of alkyl, alkoxy, haloalkyl, haloalkoxy, aryl, hydroxyl, halo, cyano, amido, and carboxyl; and
R¹⁷ and R¹⁸ are independently hydrogen or alkyl,
with the proviso that the compound is not 4′-chloro-6-((4-chlorobenzyl)oxy)-3-(1-methyl-3-(trifluoromethyl)-1H-pyrazol-5-yl)-3′-(trifluoromethyl)-[1,1′-biphenyl]-2-ol.

2. The compound of claim 1, having a formula I(a):

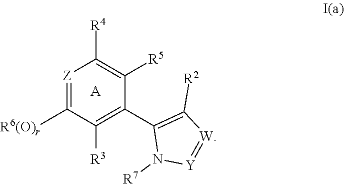

3. The compound of claim 2, wherein
R¹ is hydrogen, alkyl, aryl, benzyl, wherein R¹ is optionally substituted at one or more positions with one or more of alkyl, alkoxy, aryl, halo, —(CH₂)—NH—R⁹, or —OR¹¹;
R² is hydrogen or chloro;
R³ is hydroxyl or —OC(O)Me;
R⁴ is hydrogen or halo;
R⁵ is hydrogen or halo;
R⁶ is hydrogen, aryl, benzyl, optionally R¹ is substituted at one or more positions with one or more of haloalkyl, halo, and cyano;
R⁸ is cyano, amino, haloalkyl, or amido; and
R¹⁴ is aryl optionally substituted at one or more ring positions with one or more of haloalkyl or halo.

4. The compound of claim 3, wherein Y is N and W is CCF₃.

5. The compound of claim 4, wherein R³ is hydroxyl.

6. The compound of claim 5, wherein r is 0 and R⁶ is phenyl substituted with chloro and trifluoromethyl.

7. The compound of claim 6, wherein R² is hydrogen.

8. The compound of claim 2, wherein the compound is selected from the group consisting of:

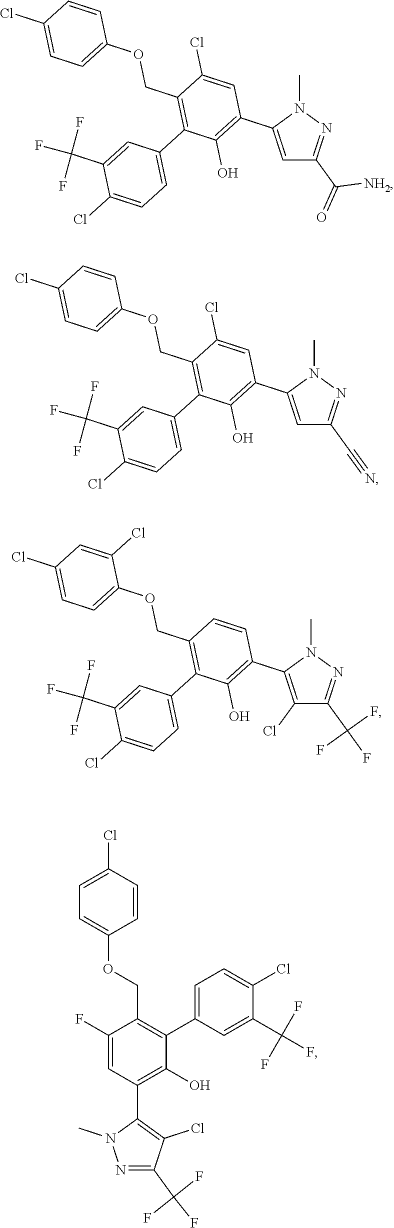 [SMILES: Cn1nc(C(N)=O)cc1-c1cc(Cl)c(COc2ccc(Cl)cc2)c(-c2ccc(Cl)c(C(F)(F)F)c2)c1O ; Cn1nc(C#N)cc1-c1cc(Cl)c(COc2ccc(Cl)cc2)c(-c2ccc(Cl)c(C(F)(F)F)c2)c1O ; Cn1nc(C(F)(F)F)c(Cl)c1-c1ccc(COc2ccc(Cl)cc2Cl)c(-c2ccc(Cl)c(C(F)(F)F)c2)c1O ; Cn1nc(C(F)(F)F)c(Cl)c1-c1cc(F)c(COc2ccc(Cl)cc2)c(-c2ccc(Cl)c(C(F)(F)F)c2)c1O]

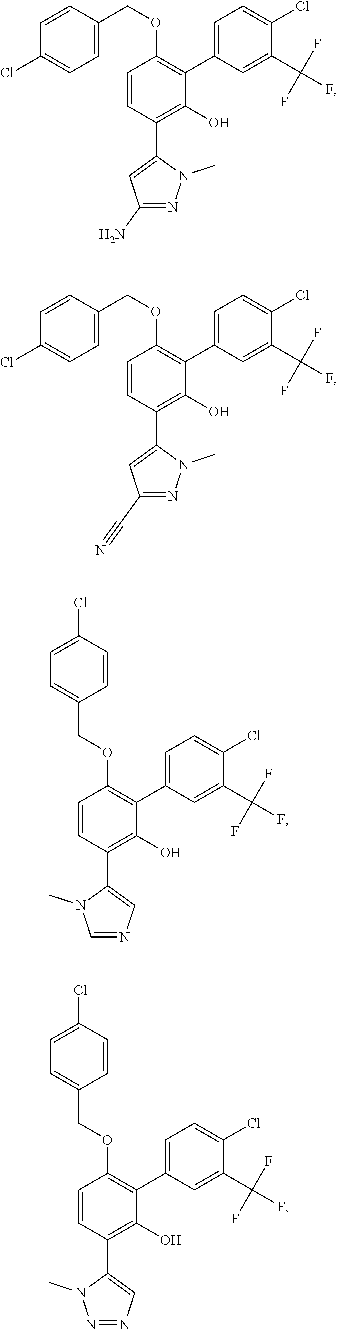 [SMILES: Cn1nc(N)cc1-c1ccc(OCc2ccc(Cl)cc2)c(-c2ccc(Cl)c(C(F)(F)F)c2)c1O ; Cn1nc(C#N)cc1-c1ccc(OCc2ccc(Cl)cc2)c(-c2ccc(Cl)c(C(F)(F)F)c2)c1O ; Cn1cncc1-c1ccc(OCc2ccc(Cl)cc2)c(-c2ccc(Cl)c(C(F)(F)F)c2)c1O ; Cn1nncc1-c1ccc(OCc2ccc(Cl)cc2)c(-c2ccc(Cl)c(C(F)(F)F)c2)c1O]

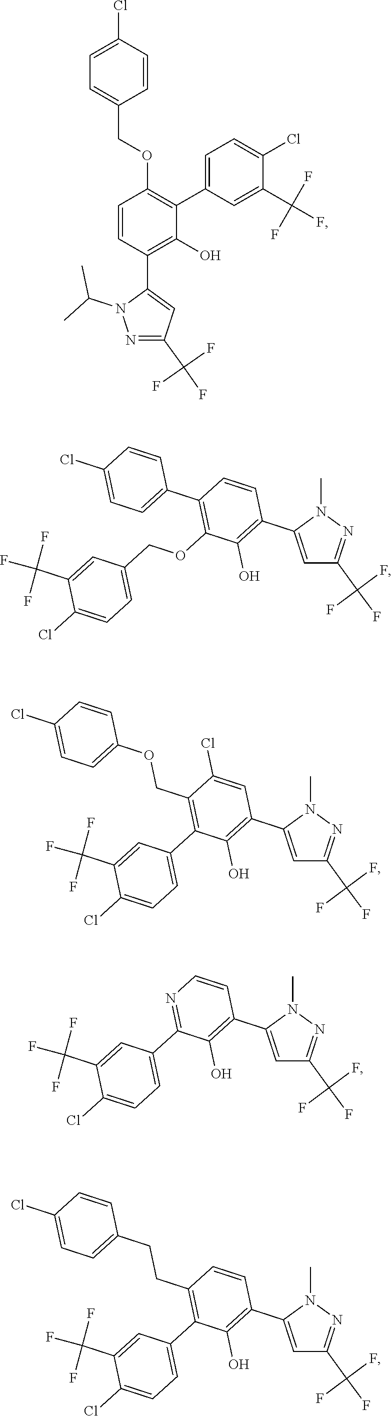 [SMILES: CC(C)n1nc(C(F)(F)F)cc1-c1ccc(OCc2ccc(Cl)cc2)c(-c2ccc(Cl)c(C(F)(F)F)c2)c1O ; Cn1nc(C(F)(F)F)cc1-c1ccc(-c2ccc(Cl)cc2)c(OCc2ccc(Cl)c(C(F)(F)F)c2)c1O ; Cn1nc(C(F)(F)F)cc1-c1cc(Cl)c(COc2ccc(Cl)cc2)c(-c2ccc(Cl)c(C(F)(F)F)c2)c1O ; Cn1nc(C(F)(F)F)cc1-c1ccnc(-c2ccc(Cl)c(C(F)(F)F)c2)c1O ; Cn1nc(C(F)(F)F)cc1-c1ccc(CCc2ccc(Cl)cc2)c(-c2ccc(Cl)c(C(F)(F)F)c2)c1O]

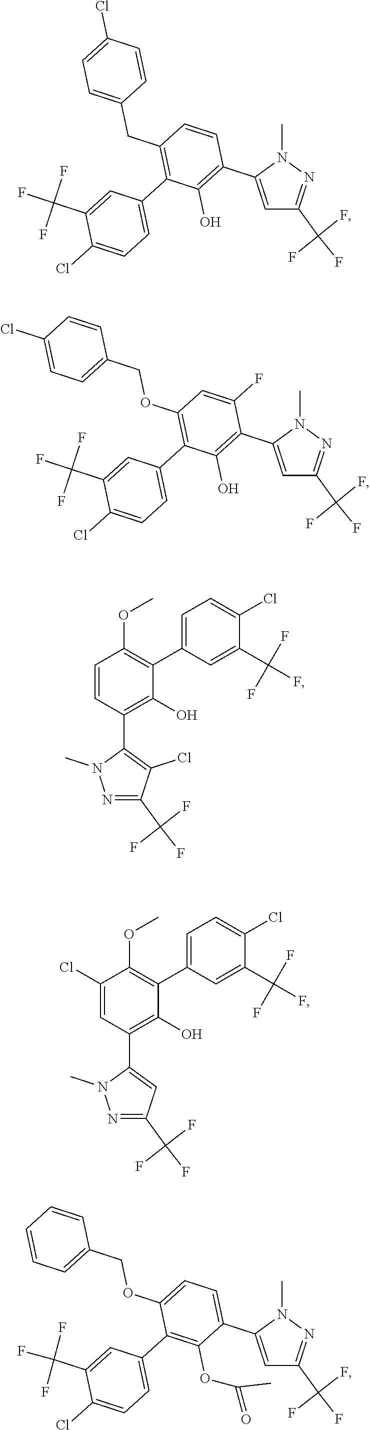 [SMILES: Cn1nc(C(F)(F)F)cc1-c1ccc(Cc2ccc(Cl)cc2)c(-c2ccc(Cl)c(C(F)(F)F)c2)c1O ; Cn1nc(C(F)(F)F)cc1-c1c(F)cc(OCc2ccc(Cl)cc2)c(-c2ccc(Cl)c(C(F)(F)F)c2)c1O ; COc1ccc(-c2c(Cl)c(C(F)(F)F)nn2C)c(O)c1-c1ccc(Cl)c(C(F)(F)F)c1 ; COc1c(Cl)cc(-c2cc(C(F)(F)F)nn2C)c(O)c1-c1ccc(Cl)c(C(F)(F)F)c1 ; CC(=O)Oc1c(-c2cc(C(F)(F)F)nn2C)ccc(OCc2ccccc2)c1-c1ccc(Cl)c(C(F)(F)F)c1]

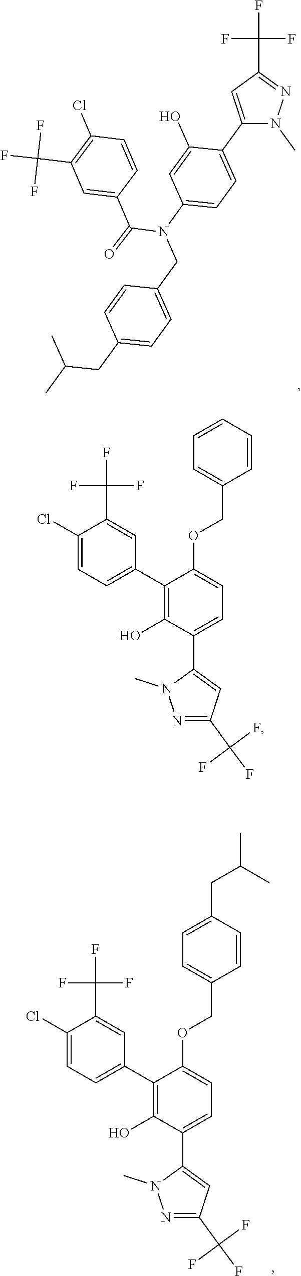 [SMILES: CC(C)Cc1ccc(CN(C(=O)c2ccc(Cl)c(C(F)(F)F)c2)c2ccc(-c3cc(C(F)(F)F)nn3C)c(O)c2)cc1 ; Cn1nc(C(F)(F)F)cc1-c1ccc(OCc2ccccc2)c(-c2ccc(Cl)c(C(F)(F)F)c2)c1O ; CC(C)Cc1ccc(COc2ccc(-c3cc(C(F)(F)F)nn3C)c(O)c2-c2ccc(Cl)c(C(F)(F)F)c2)cc1]

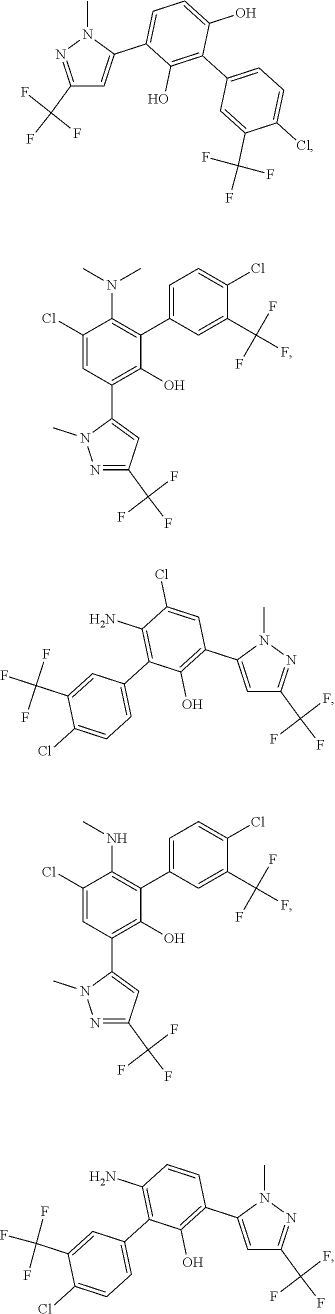 [SMILES: Cn1nc(C(F)(F)F)cc1-c1ccc(O)c(-c2ccc(Cl)c(C(F)(F)F)c2)c1O ; CN(C)c1c(Cl)cc(-c2cc(C(F)(F)F)nn2C)c(O)c1-c1ccc(Cl)c(C(F)(F)F)c1 ; Cn1nc(C(F)(F)F)cc1-c1cc(Cl)c(N)c(-c2ccc(Cl)c(C(F)(F)F)c2)c1O ; CNc1c(Cl)cc(-c2cc(C(F)(F)F)nn2C)c(O)c1-c1ccc(Cl)c(C(F)(F)F)c1 ; Cn1nc(C(F)(F)F)cc1-c1ccc(N)c(-c2ccc(Cl)c(C(F)(F)F)c2)c1O]

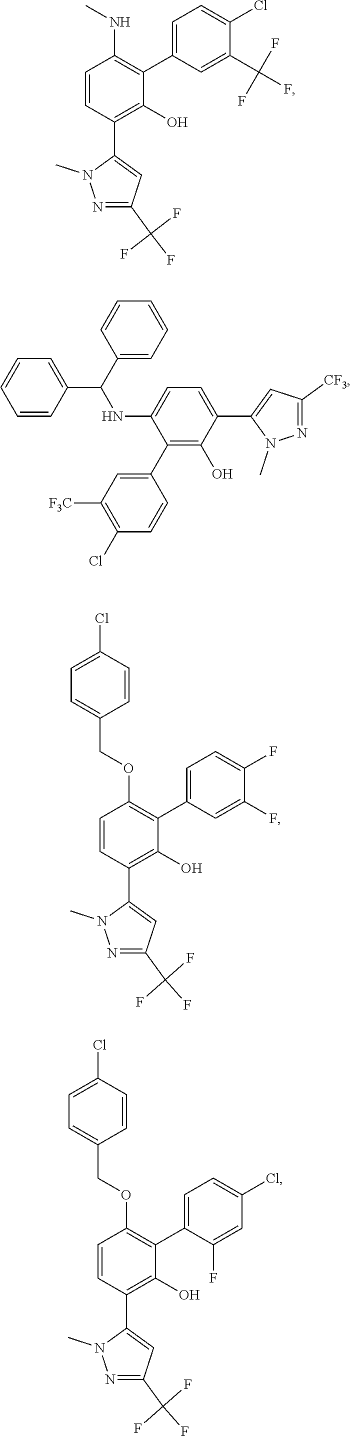

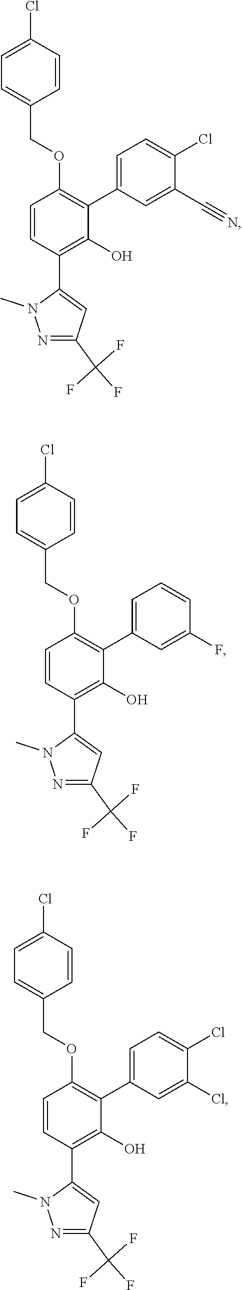 [SMILES: Cn1nc(C(F)(F)F)cc1-c1ccc(OCc2ccc(Cl)cc2)c(-c2ccc(Cl)c(C#N)c2)c1O ; Cn1nc(C(F)(F)F)cc1-c1ccc(OCc2ccc(Cl)cc2)c(-c2cccc(F)c2)c1O ; Cn1nc(C(F)(F)F)cc1-c1ccc(OCc2ccc(Cl)cc2)c(-c2ccc(Cl)c(Cl)c2)c1O]

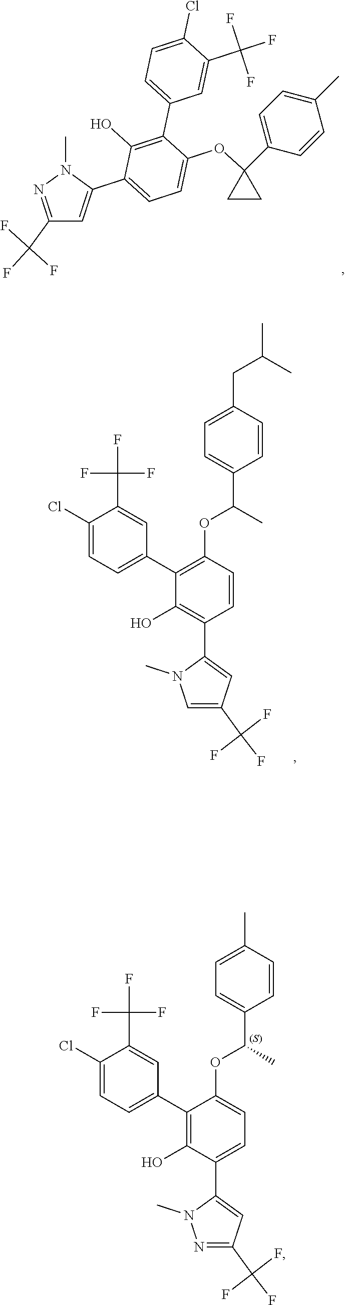

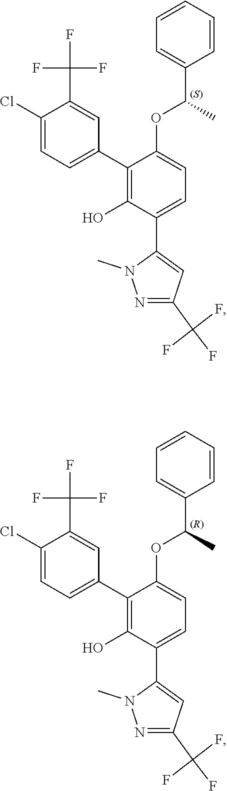

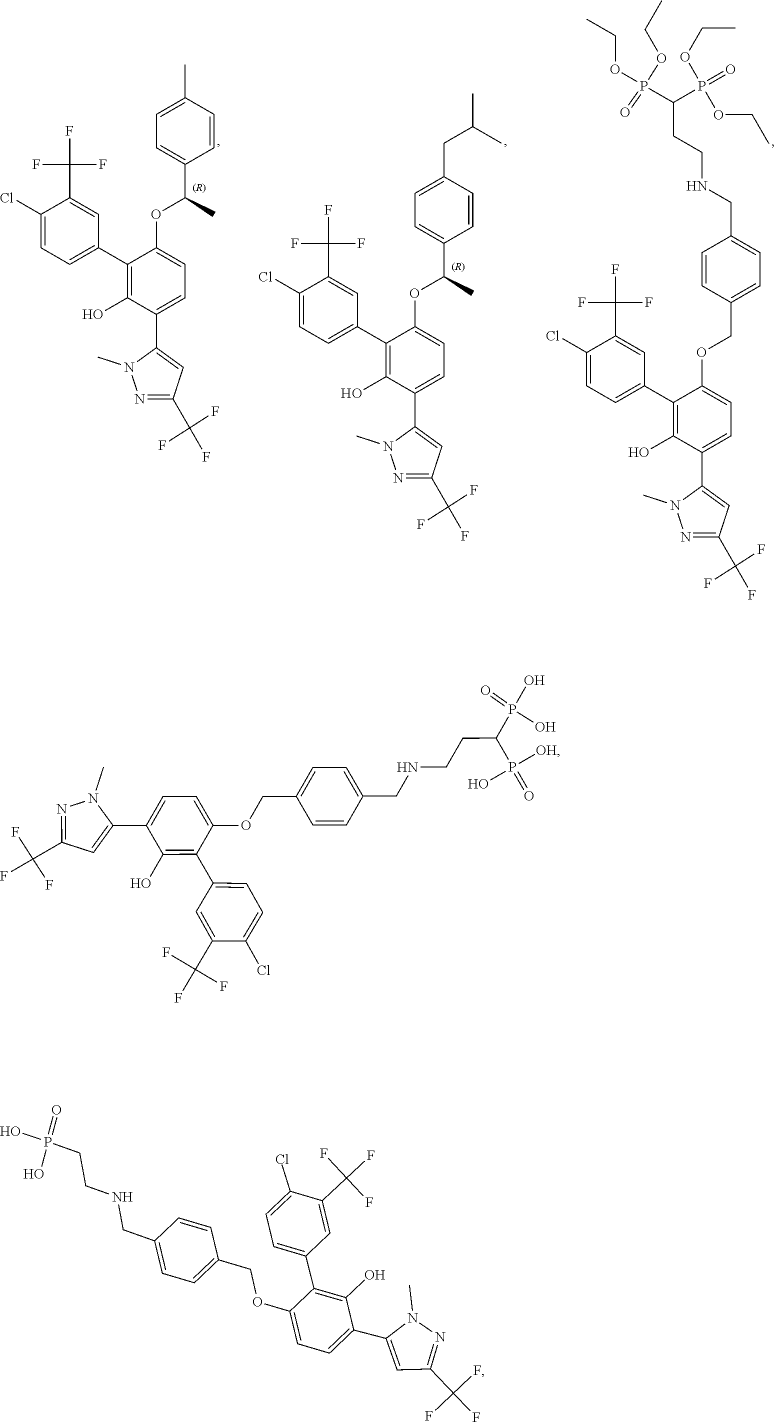

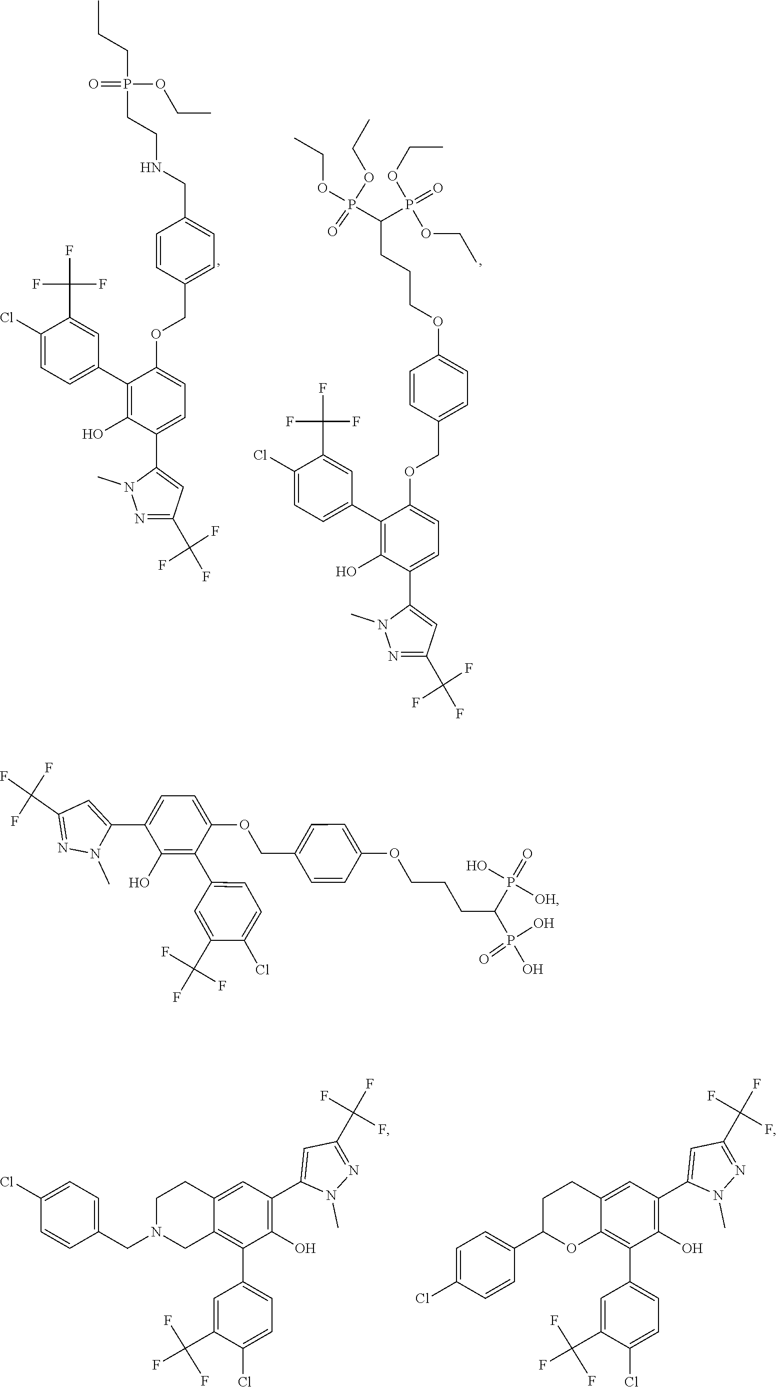 [SMILES: CCCP(=O)(CCNCc1ccc(COc2ccc(-c3cc(C(F)(F)F)nn3C)c(O)c2-c2ccc(Cl)c(C(F)(F)F)c2)cc1)OCC ; CCOP(=O)(OCC)C(CCCOc1ccc(COc2ccc(-c3cc(C(F)(F)F)nn3C)c(O)c2-c2ccc(Cl)c(C(F)(F)F)c2)cc1)P(=O)(OCC)OCC ; Cn1nc(C(F)(F)F)cc1-c1ccc(OCc2ccc(OCCCC(P(=O)(O)O)P(=O)(O)O)cc2)c(-c2ccc(Cl)c(C(F)(F)F)c2)c1O ; Cn1nc(C(F)(F)F)cc1-c1cc2c(c(-c3ccc(Cl)c(C(F)(F)F)c3)c1O)CN(Cc1ccc(Cl)cc1)CC2 ; Cn1nc(C(F)(F)F)cc1-c1cc2c(c(-c3ccc(Cl)c(C(F)(F)F)c3)c1O)OC(c1ccc(Cl)cc1)CC2]

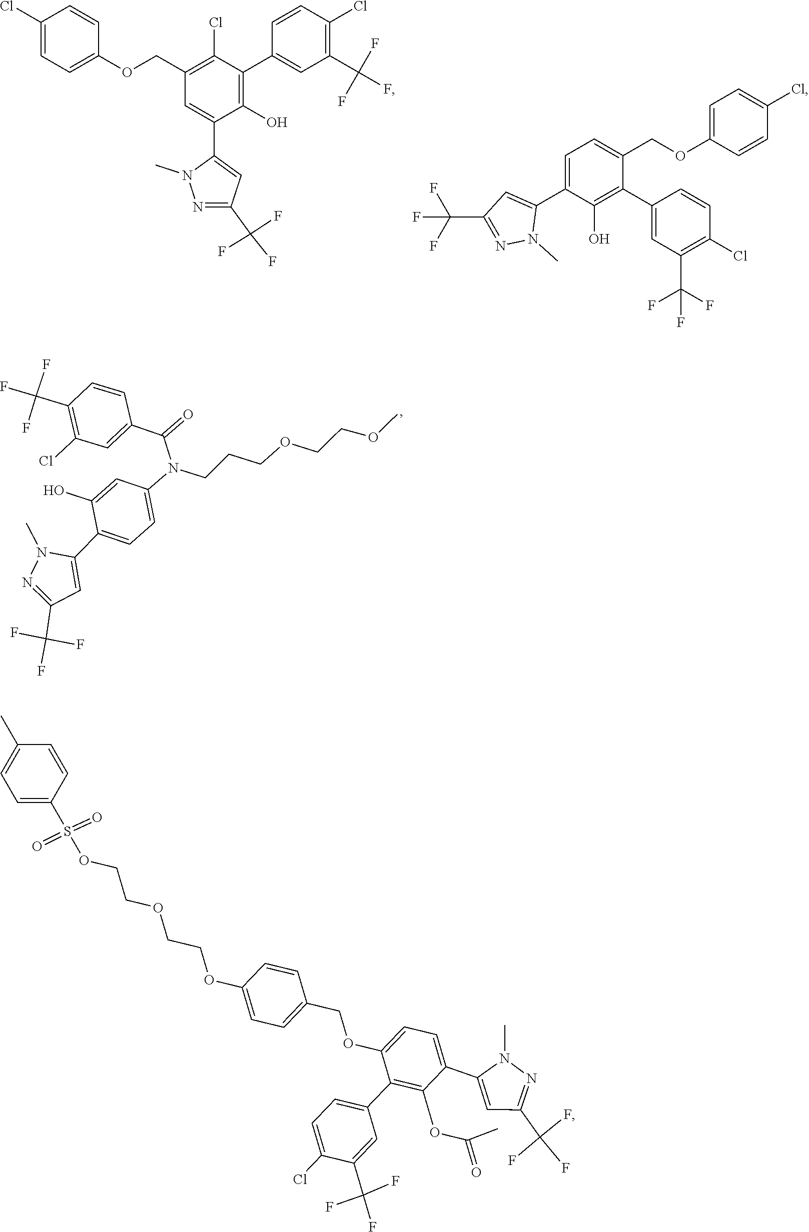

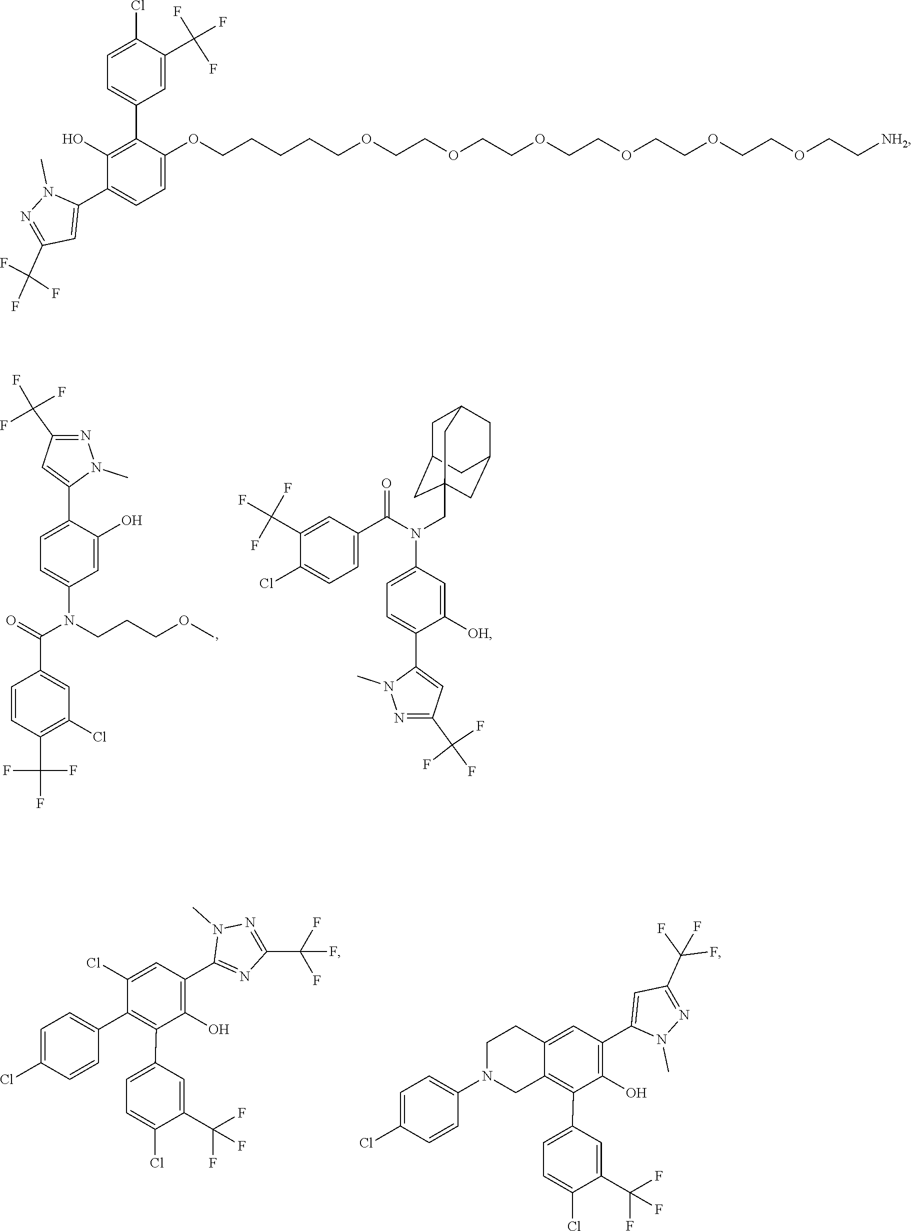 [SMILES: Cn1nc(C(F)(F)F)cc1-c1ccc(OCCCCCOCCOCCOCCOCCOCCOCCN)c(-c2ccc(Cl)c(C(F)(F)F)c2)c1O ; COCCCN(C(=O)c1ccc(C(F)(F)F)c(Cl)c1)c1ccc(-c2cc(C(F)(F)F)nn2C)c(O)c1 ; Cn1nc(C(F)(F)F)cc1-c1ccc(N(CC23CC4CC(CC(C4)C2)C3)C(=O)c2ccc(Cl)c(C(F)(F)F)c2)cc1O ; Cn1nc(C(F)(F)F)nc1-c1cc(Cl)c(-c2ccc(Cl)cc2)c(-c2ccc(Cl)c(C(F)(F)F)c2)c1O ; Cn1nc(C(F)(F)F)cc1-c1cc2c(c(-c3ccc(Cl)c(C(F)(F)F)c3)c1O)CN(c1ccc(Cl)cc1)CC2]

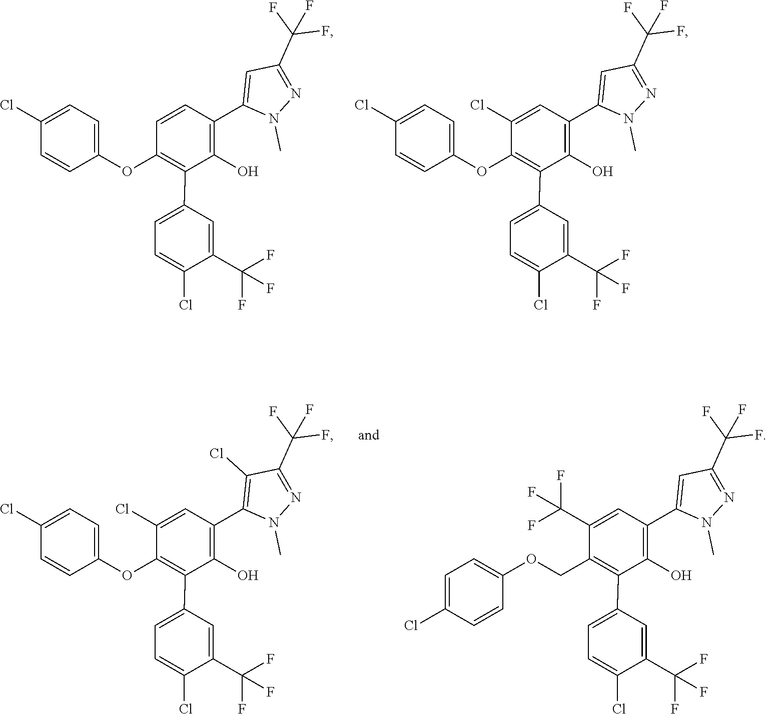 [SMILES: Cn1nc(C(F)(F)F)cc1-c1ccc(Oc2ccc(Cl)cc2)c(-c2ccc(Cl)c(C(F)(F)F)c2)c1O ; Cn1nc(C(F)(F)F)cc1-c1cc(Cl)c(Oc2ccc(Cl)cc2)c(-c2ccc(Cl)c(C(F)(F)F)c2)c1O ; Cn1nc(C(F)(F)F)c(Cl)c1-c1cc(Cl)c(Oc2ccc(Cl)cc2)c(-c2ccc(Cl)c(C(F)(F)F)c2)c1O ; "and" ; Cn1nc(C(F)(F)F)cc1-c1cc(C(F)(F)F)c(COc2ccc(Cl)cc2)c(-c2ccc(Cl)c(C(F)(F)F)c2)c1O]

9. A compound having a formula II: 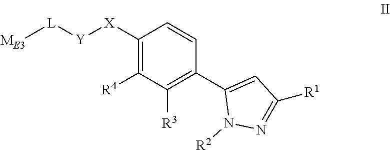
wherein
R¹ is hydrogen, cyano, amino, alkyl, alkoxy, haloalkyl, haloalkoxy, aryl, benzyl, hydroxyl, halo, amido, and carboxyl;
R² is alkyl;
R³ is alkyl, alkoxy, haloalkyl, haloalkoxy, hydroxyl, —O—C(O)-alkyl, or halo;
R⁴ is hydrogen, halo, alkyl, aryl, alkylaryl, heteroaryl, cycloalkyl, or cycloheteroalkyl, optionally R⁴ is substituted at one or more positions with one or more of alkyl, alkoxy, haloalkyl, haloalkoxy, hydroxyl, halo, cyano, carboxyamido, carboxy, aryloxy, and heteroaryloxy;
X is O or NR⁵;
R⁵ is hydrogen, alkyl, or —C(O)R⁶;
R⁶ is aryl optionally substituted at one or more ring positions with one or more of alkyl, alkoxy, haloalkyl, haloalkoxy, aryl, hydroxyl, halo, cyano, amido, and carboxyl;
Y is alkylenyl, arylenyl, benzylenyl, heteroarylenyl, cycloalkylenyl, or cycloheteroalkylenyl;
L is a bond, or a linker selected from the group consisting of —(O(CH₂)₂)ₐ—, —(O(CH₂)₂)ₐ—NH—C(O)-Alk³-, —(O(CH₂)₂)ₐ—C(O)—NH—(CH₂)₂—O)_{b}-Alk³-, —(O(CH₂)₂)ₐ—C(O)—NH-Alk³-C(O)—, —(((CH₂)₂)ₐ—C(O)—NH—((CH₂)₂—O)_{b}—, —(O(CH₂)₂)ₐ—C(O)—NH—((CH₂)₂—O)_{b}-Alk³-C(O)—, —(((CH₂)₂)ₐ—C(O)—NH-Alk³-NH—C(O)—CH₂—, and —(O(CH₂)₂)ₐ—C(O)—NH-Alk³-;
a is an integer selected from 1-20;
b is an integer selected from 1-20;
Alk³ is straight-chain or branched alkylenyl;
M_{E3} is selected from the group consisting of 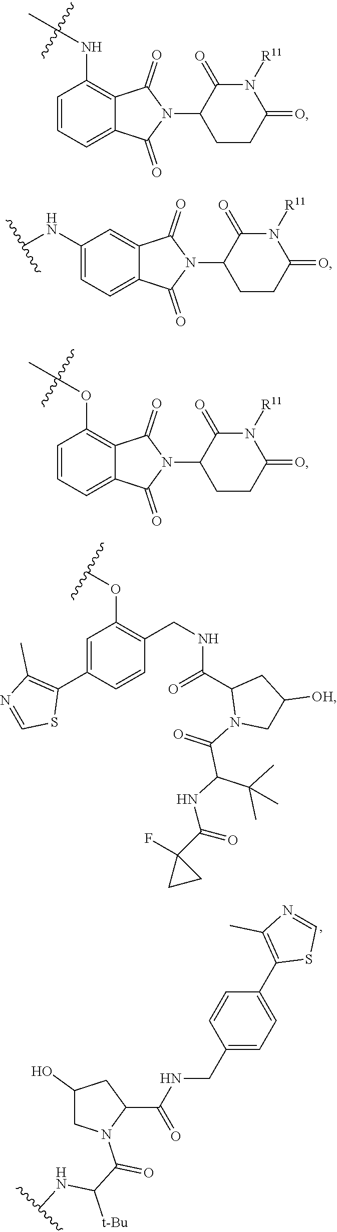 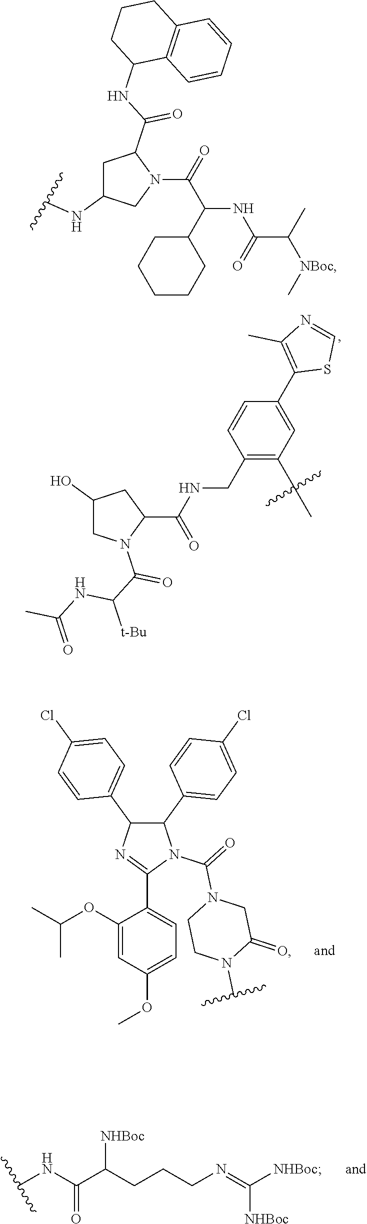
R¹¹ is hydrogen or alkyl.

10. The compound of claim 9, wherein the compound has a structure of formula II(a): 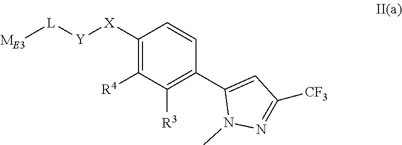
wherein
R³ is hydroxyl or —O—C(O)-alkyl;
R⁴ is hydrogen or phenyl substituted at one or more positions with one or more of haloalkyl or halo;
X is O or —N—C(O)R⁶;
R⁶ is aryl substituted at one or more ring positions with one or more of haloalkyl or halo;
Y is alkylenyl or benzylenyl;
L is a bond, or a linker selected from the group consisting of —(O(CH₂)₂)ₐ—, —(O(CH₂)₂)ₐ—C(O)—NH—((CH₂)₂—O)_{b}-Alk³-, —(O(CH₂)₂)ₐ—C(O)—NH-Alk³-C(O)—, —(O(CH₂)₂)ₐ—C(O)—NH—((CH₂)₂—O)_{b}—, —(O(CH₂)₂)ₐ—C(O)—NH—((CH₂)₂—O)_{b}-Alk³-C(O)—, —(O(CH₂)₂)ₐ—C(O)—NH-Alk³-NH—C(O)—CH₂—, and —(((CH₂)₂)ₐ—C(O)—NH-Alk³-; and
R⁸ is alkyl or aryl, wherein R⁸ is optionally substituted at one or more positions with one or more of alkyl.

11. The compound of claim 10, wherein R³ is hydroxyl, and R⁴ is phenyl substituted with trifluoromethyl and chloro.

12. The compound of claim 11, wherein X is —N—C(O)R⁶, and R⁶ is phenyl substituted with trifluoromethyl and chloro.

13. The compound of claim 11, wherein X is O, and Y is propylenyl or benzylenyl.

14. The compound of claim 9, wherein the compound is selected from the group consisting of:

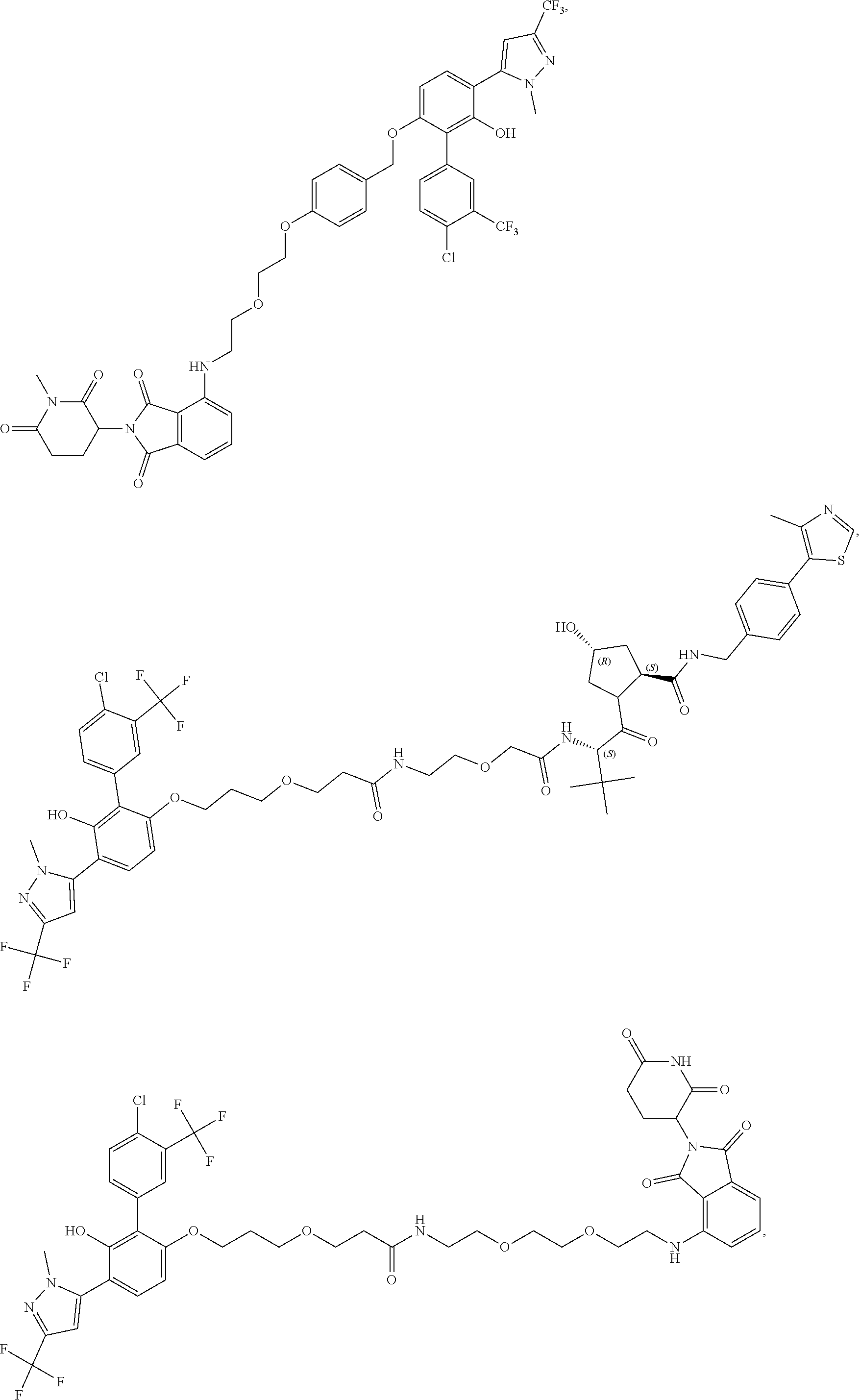

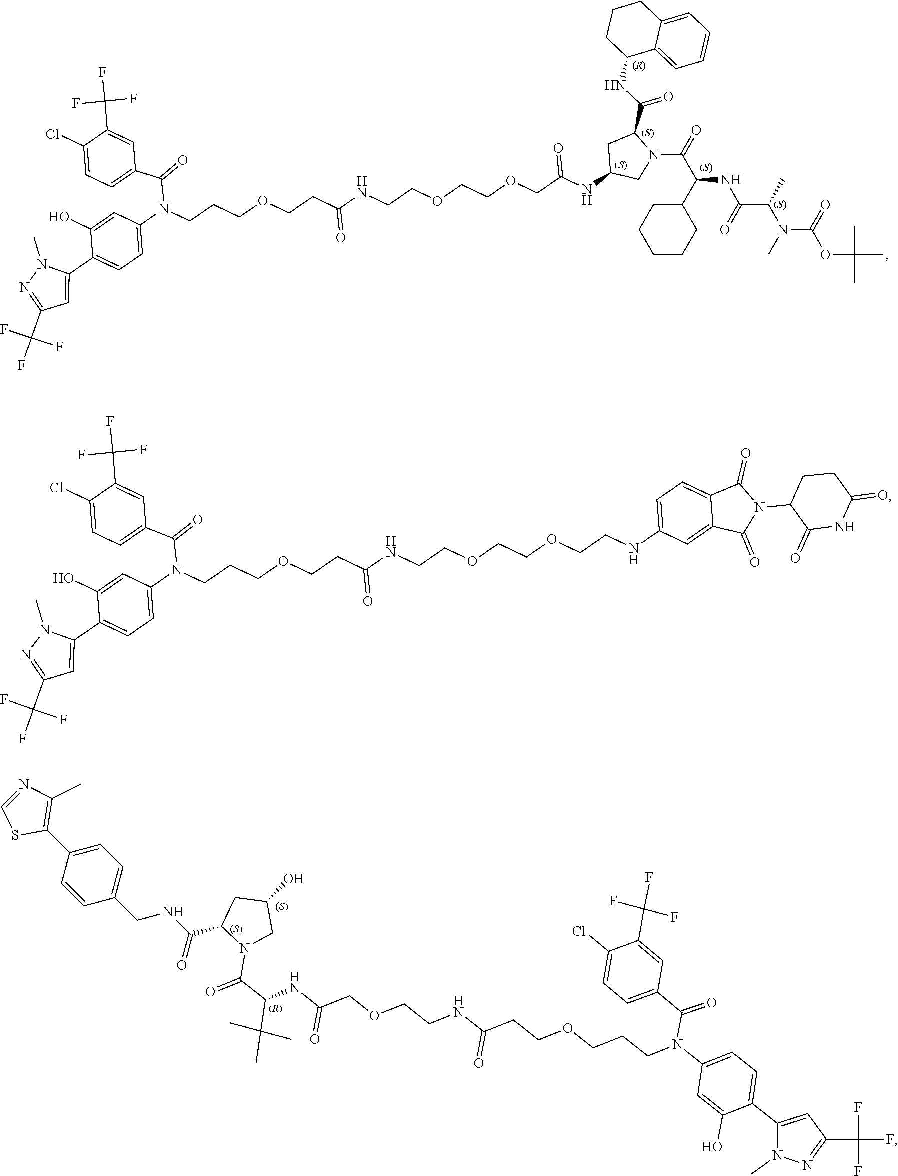

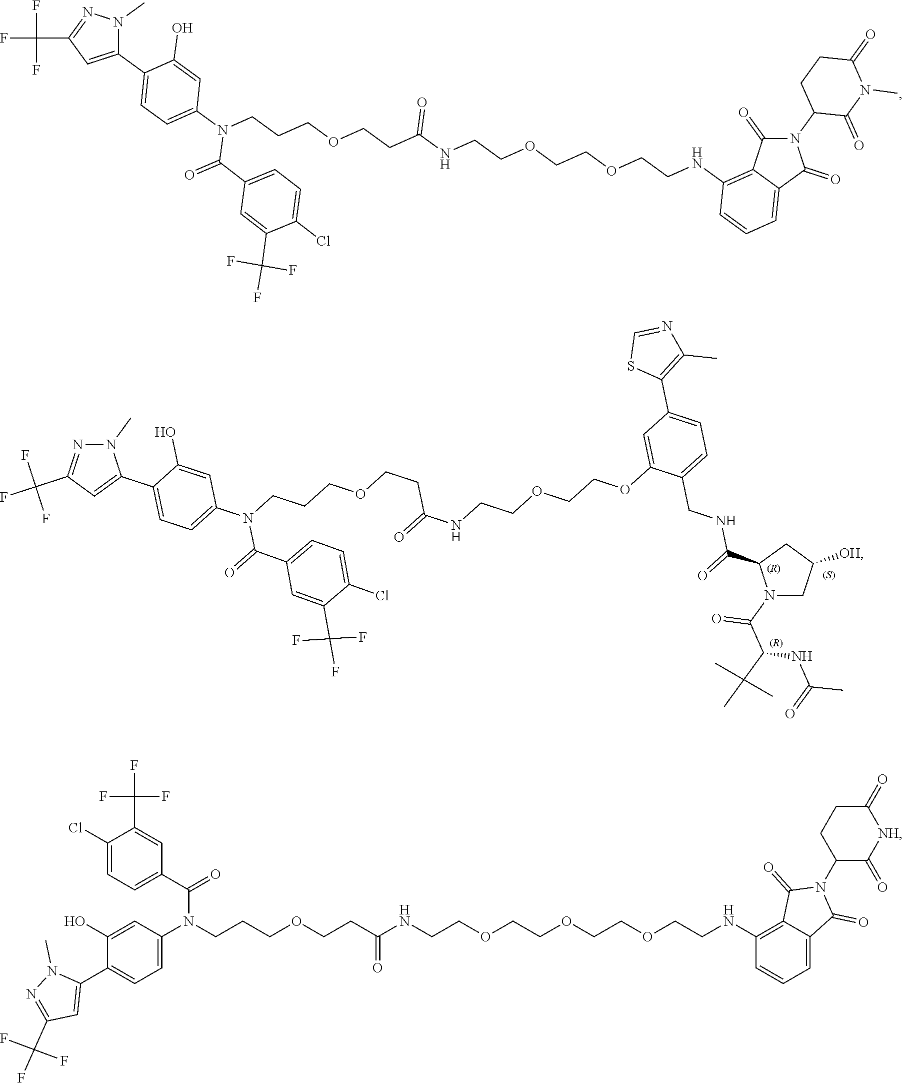

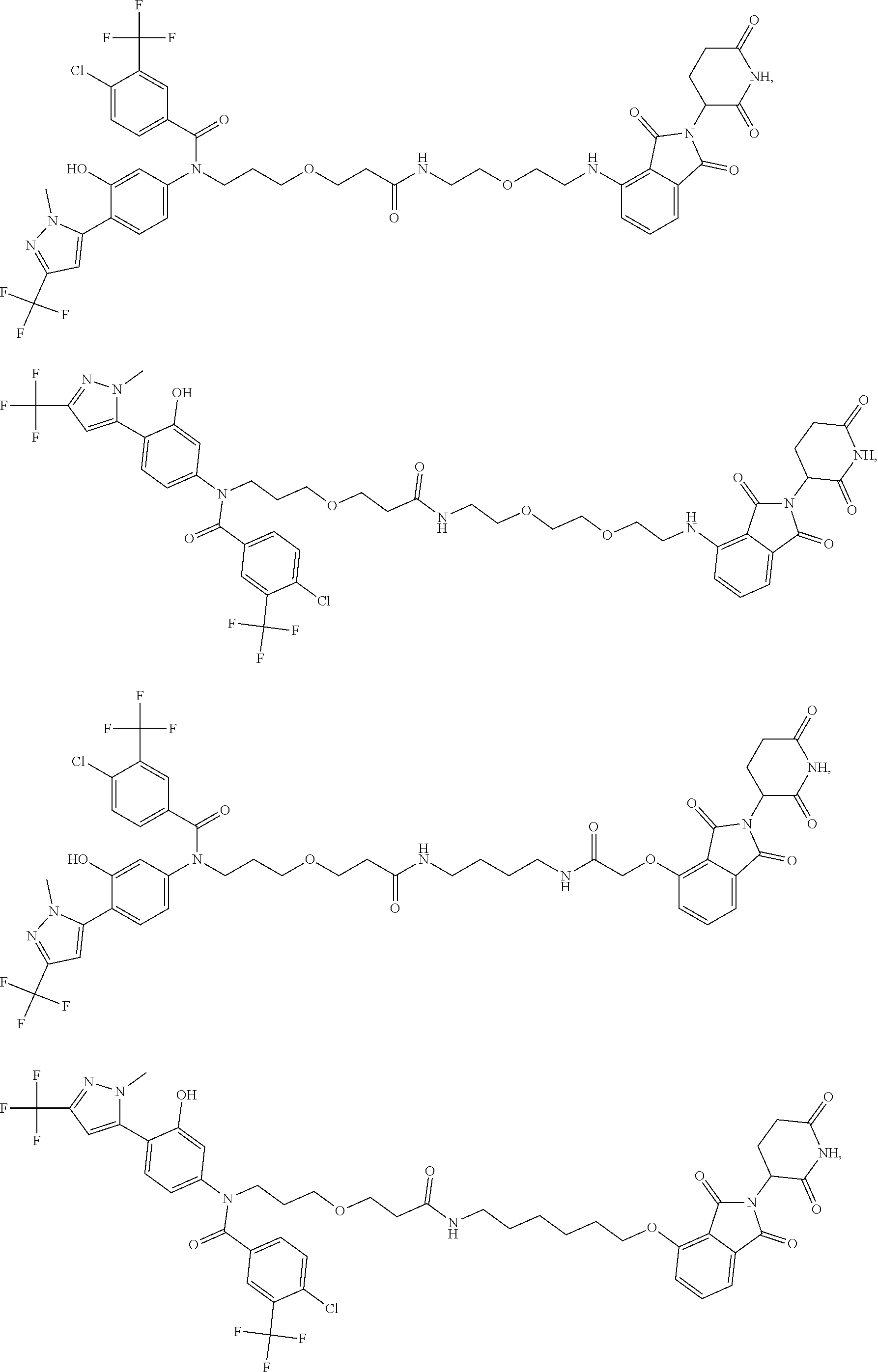

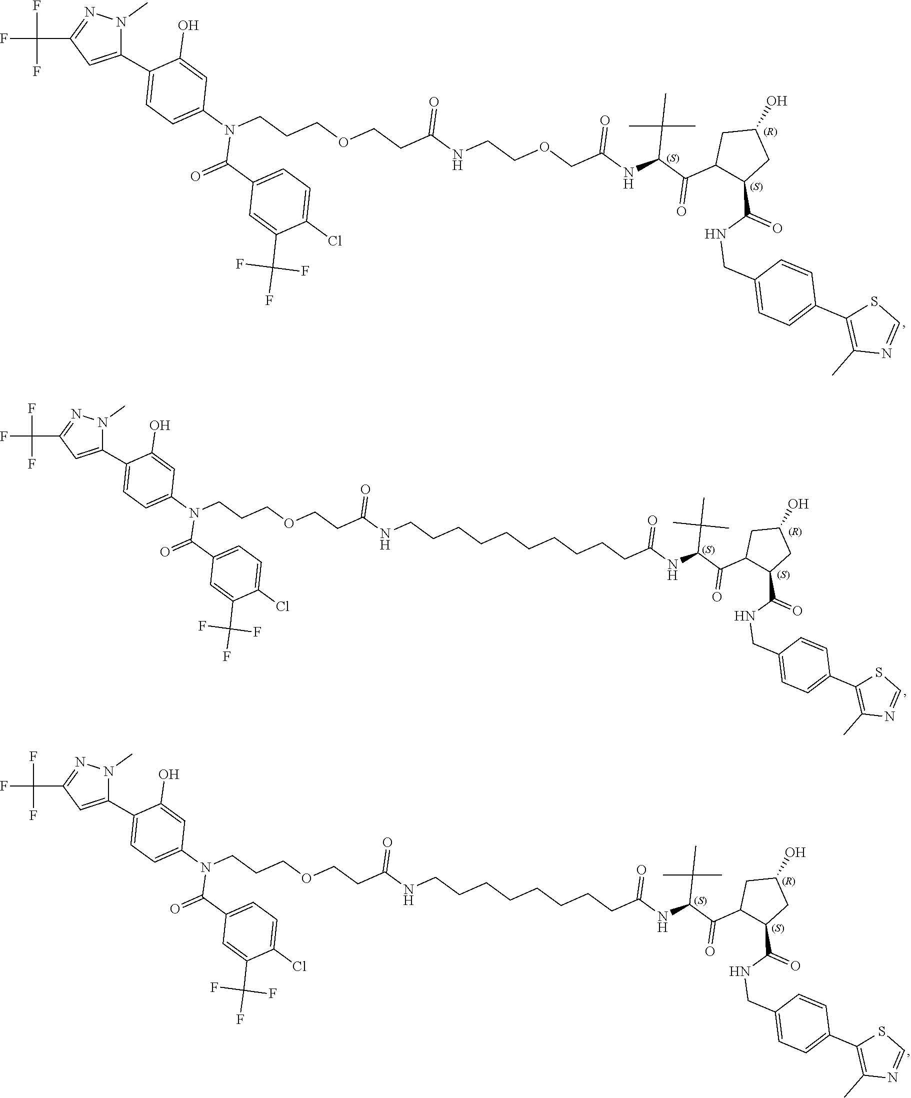

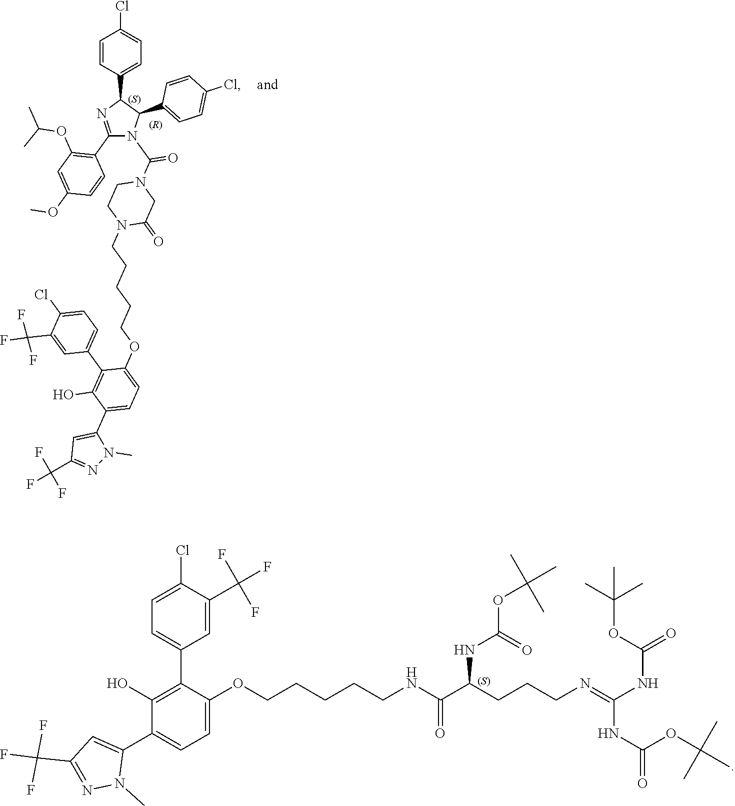

15. A pharmaceutical composition comprising a molecule of claim 1 and a suitable pharmaceutical carrier, excipient, or diluent.

16. A method of treating cancer, the method comprising administering the composition of claim 15 to a subject having the cancer.

17. The method of claim 16, wherein the cancer is selected from multiple myeloma, leukemia, non-small cell lung cancer, colon cancer, cancer of the central nervous system, melanoma, ovarian cancer, renal cancer, prostate cancer, and breast cancer.

18. A pharmaceutical composition comprising a molecule of claim 9 and a suitable pharmaceutical carrier, excipient, or diluent.

19. A method of treating cancer, the method comprising administering the composition of claim 18 to a subject having the cancer.

20. The method of claim 19, wherein the cancer is selected from multiple myeloma, leukemia, non-small cell lung cancer, colon cancer, cancer of the central nervous system, melanoma, ovarian cancer, renal cancer, prostate cancer, and breast cancer.
